(19) United States
(12) Patent Application Publication
(10) Pub. No.: US 2024/0123080 A1
(43) Pub. Date: Apr. 18, 2024

(54) CYTOTOXIC COMPOUNDS AND CONJUGATES THEREOF

(71) Applicant: Seagen Inc., Bothell, WA (US)

(72) Inventors: Philip MOQUIST, Bothell, WA (US); Svetlana DORONINA, Bothell, WA (US); Nicole DUNCAN, Bothell, WA (US); Gabriele BLAHNIK-FAGAN, Bothell, WA (US); Sharsti SANDALL, Bothell, WA (US)

(21) Appl. No.: 18/448,863

(22) Filed: Aug. 11, 2023

Related U.S. Application Data
(60) Provisional application No. 63/397,776, filed on Aug. 12, 2022.

(30) Foreign Application Priority Data
Oct. 18, 2022 (EP) 22202077.8

(51) Int. Cl. A61K 47/68; A61K 47/54; A61P 35/00; C07K 5/103

(52) U.S. Cl. CPC A61K 47/68031; A61K 47/543; A61K 47/545; A61K 47/549; A61K 47/6851; A61K 47/6889; A61P 35/00; C07K 5/101

(57) ABSTRACT

Hydrophilic auristatin compounds are described, including Drug-Linker compounds, Ligand-Drug Conjugate compounds, methods of use, and preparations thereof.

### CROSS REFERENCE TO RELATED APPLICATIONS

This patent application claims the priority benefit of U.S. Provisional Application No. 63/397,776, filed on Aug. 12, 2022, and EP Application No. 22202077.8, filed on Oct. 18, 2022, the contents of each of which are incorporated herein by reference in their entirety.

### REFERENCE TO AN ELECTRONIC SEQUENCE LISTING

The contents of the electronic sequence listing (761682007600SEQLIST.xml; Size: 966,947 bytes; and Date of Creation: Aug. 9, 2023) are herein incorporated by reference in their entirety.

### BACKGROUND OF THE INVENTION

A variety of ligands have been investigated for the targeted delivery of cytotoxic agents to tumor cells, including oligopeptides, antibodies, and other proteins. While various drug classes have been evaluated for targeted delivery by these ligands, only a few drug classes have proved sufficiently active as Ligand-Drug Conjugates, while having a suitable toxicity profile and other pharmacological properties, to warrant clinical development. There is thus a need for additional cytotoxic agents with improved toxicity profile and other pharmacological properties.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 4th edition (2012) Cold Spring Harbor Laboratory Press, Cold Spring Harbor N.Y.; Current Protocols In Molecular Biology (F. M. Ausubel, et al. eds., (2003)); the series METHODS IN ENZYMOLOGY (Academic Press, Inc.); PCR 2: A Practical Approach (M. J. MacPherson, B. D. Hames and G. R. Taylor eds. (1995)); Greenfield, ed. (2013) Antibodies, A Laboratory Manual, 2^{nd} edition, Cold Spring Harbor Laboratory Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); Cancer: Principles and Practice of Oncology (V. T. DeVita et al., eds., J.B. Lippincott Company, 1993); and updated versions thereof. Each of the foregoing references in this paragraph is incorporated herein by reference in its entirety.

### BRIEF SUMMARY OF THE INVENTION

Provided herein is a compound of Formula (I):

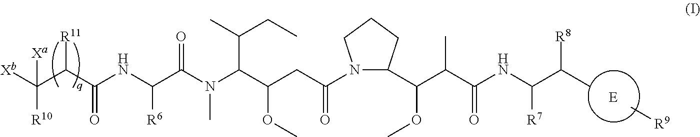

or a salt thereof, wherein
X^{b} is —NR¹R²; and X^{a} is

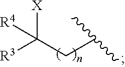

or
X^{a} and X^{b} are taken together with the carbon atom to which they are attached to form

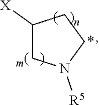

wherein the asterisk denotes the carbon atom of Formula (I) that bears the X^{a} and X^{b} groups;
R¹, R², R³, R⁴, R^{a}, R^{b}, R⁵, and R¹⁰ are each independently H or C₁-C₄ alkyl;
X is H, OH, —C(O)NR^{a}R^{b}, —S(O)₂R^{a}, —S(O)—R^{a}, —S(O)₂NR^{a}R^{b}, —NHS(O)₂R^{a}, or —NHC(O)R^{a};
R⁶ is C₁-C₄ alkyl optionally substituted with OH;
R⁷ is H, C₁-C₄ alkyl optionally substituted with one or two OH moieties, or 5-6 membered heteroaryl;
E is phenyl or 5-6 membered heteroaryl;
R⁸, R⁹, and R¹¹ are each independently H or OH;
n is 0, 1, 2, or 3;
m is 1, 2, 3, or 4; and
q is 0 or 1,
wherein when X is H, at least two of R⁷, R⁸, R⁹, and R¹¹ comprise an OH moiety.

Also provided herein is a compound of Formula (II):

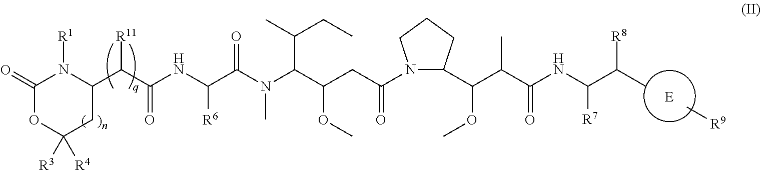

or a salt thereof, wherein
R¹, R³, and R⁴ are independently H or C₁-C₄ alkyl;
R⁶ is C₁-C₄ alkyl optionally substituted with OH;
R⁷ is H, C₁-C₄ alkyl optionally substituted with one or two OH moieties, or 5-6 membered heteroaryl;
E is phenyl or 5-6 membered heteroaryl;
R⁸, R⁹, and R¹¹ are each independently H or OH;
n is 0, 1, or 2; and
q is 0 or 1.

In some embodiments of Formula (I) and (II), q is 0. In some embodiments, q is 1. In some embodiments, R¹¹ is H. In some embodiments, R¹¹ is OH.

In some embodiments of Formula (I), X is OH. In some embodiments, X is —C(O)NR^{a}R^{b}, —S(O)₂R^{a}, —S(O)—R^{a} —S(O)₂NR^{a}R^{b}, —NHS(O)₂R^{a}, or —NHC(O)R^{a}. In some embodiments, X^{b} is —NR¹R² and X^{a} is

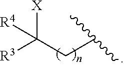

In some embodiments, R³ is H. In some embodiments, R⁴ is H. In some embodiments, n is 0 or 1. In some embodiments, X^{a} and X^{b} are taken together with the carbon atom to which they are attached to form

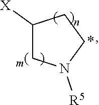

wherein the asterisk denotes the carbon atom of Formula (I) that bears the X^{a} and X^{b} groups. In some embodiments, m is 2 or 3.

In some embodiments of Formula (I) or (II), R¹ is H. In some embodiments, R¹ is C₁-C₄ alkyl. In some embodiments, R¹ is methyl. In some embodiments, R² is methyl. In some embodiments, R¹⁰ is H. In some embodiments, R¹⁰ is methyl. In some embodiments, R⁶ is unsubstituted C₁-C₄ alkyl. In some embodiments, R⁶ is isopropyl. In some embodiments, R⁶ is C₁-C₄ alkyl substituted with OH. In some embodiments, R⁷ C₁-C₄ alkyl substituted with OH. In some embodiments, R⁷ is —CH₂OH. In some embodiments, R⁷ is H. In some embodiments, R⁷ is unsubstituted C₁-C₄ alkyl. In some embodiments, R⁷ is methyl. In some embodiments, R⁷ is 5-6 membered heteroaryl. In some embodiments, R⁸ is H. In some embodiments, R⁸ is OH. In some embodiments, E is phenyl. In some embodiments, E-R⁹ is

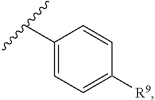

wherein the wavy line indicates the point of attachment of E to the rest of the compound. In some embodiments, E is 5-6 membered heteroaryl. In some embodiments, R⁹ is H. In some embodiments, R⁹ is OH.

In some embodiments of Formula (I),
X^{a} is

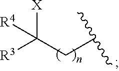

X^{b} is —NR¹R²;
R¹ is H or methyl;
R² is methyl;
X is OH;
R³ and R⁴ are H;
R⁶ is isopropyl;
R⁷ is —CH₂OH;
R⁸ is H;
E is phenyl; and
R⁹ is H.

In some embodiments of Formula (I), the compound is

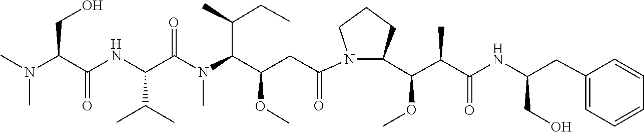 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)N(C)C)C(C)C]

or a salt thereof.

In some embodiments, the compound is a compound of Table 1.

Also provided herein is a Drug-Linker compound of the following formula:

Q-D,

or a salt thereof, wherein
Q is a Linker Unit selected from the group consisting of:
   (i) Z′-A-RL-,
   (ii) Z′-A-RL-Y—,
   (iii) Z′-A-S*-RL-,
   (iv) Z′-A-S*-RL-Y—,
   (v) Z′-A-B(S*)—RL-,
   (vi) Z′-A-B(S*)—RL-Y—,
   (vii) Z′-A-,
   (viii) Z′-A-S*—W—,
   (ix) Z′-A-B(S*)—W—,
   (x) Z′-A-S*—W-RL-, and
   (xi) Z′-A-B(S*)—W-RL-;
Z′ is a Stretcher Unit precursor;
A is a bond or a Connector Unit;
B is a Parallel Connector Unit;
S* is a Partitioning Agent;
RL is a Releasable Linker;
W is an Amino Acid Unit;
Y is a Spacer Unit; and
D is a Drug Unit of Formula (I′):

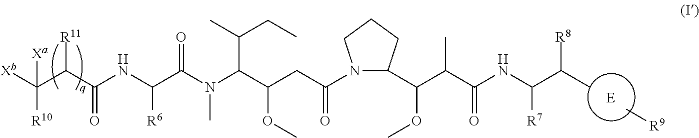

wherein
X^{b} is —NR²—^{#} or —N⁺R¹R⁵—^{#}, wherein the # denotes the point of attachment to Q, and X^{a} is

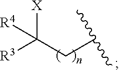

or
X^{a} and X^{b} are taken together with the carbon atom to which they are attached to form

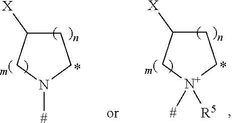

wherein the asterisk denotes the carbon atom of Formula (I) that bears the X^{a} and X^{b} groups, and the # denotes the point of attachment to Q;
R¹ and R⁵ are independently C₁-C₄ alkyl;
X is H, OH, —C(O)NR^{a}R^{b}, —S(O)₂R^{a}, —S(O)—R^{a} —S(O)₂NR^{a}R^{b}, —NHS(O)₂R^{a}, or —NHC(O)R^{a};
R², R³, R⁴, R¹⁰, R^{a}, and R^{b} are each independently H or C₁-C₄ alkyl;
R⁶ is C₁-C₄ alkyl optionally substituted with OH;
R⁷ is H, C₁-C₄ alkyl optionally substituted with one or two OH moieties, or 5-6 membered heteroaryl;
E is phenyl or 5-6 membered heteroaryl;
R⁸, R⁹, and R¹¹ are each independently H or OH;
n is 0, 1, 2, or 3;
m is 1, 2, 3, or 4;
q is 0 or 1; and
wherein when X is H, at least two of R⁷, R⁸, R⁹, and R¹¹ comprise an OH moiety.

In some embodiments, the Linker Unit Q is of formula (i), (ii), (iii), (iv), (x), or (xi). In some embodiments, the Linker Unit Q is of formula (v), (vi), (ix), or (xi). In some embodiments, the Linker Unit Q is of formula (viii), (ix), (x), or (xi).

In some embodiments, the Stretcher Unit Z′ is

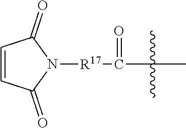

wherein
R¹⁷ is —CH₂CH₂(OCH₂CH₂)ₖ—, —C₁-C₁₀ alkylene-, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₁₀ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—;
subscript k is an integer ranging from 1 to 36;
R¹⁷ is optionally substituted by a Basic Unit (BU) such as an aminoalkyl moiety, e.g. —(CH₂)ₓNH₂, —(CH₂)ₓNHR^{a}, and —(CH₂)ₓNR^{a}₂, wherein x is an integer of from 1-4 and each R^{a} is independently selected from the group consisting of C₁₋₆ alkyl and C₁₋₆ haloalkyl, or two R^{a} groups are combined with the nitrogen to which they are attached to form an azetidinyl, pyrrolidinyl or piperidinyl group; and
the wavy line indicates the point of covalent attachment to the rest of the Drug-Linker compound.

In some embodiments, the Stretcher Unit Z′ is

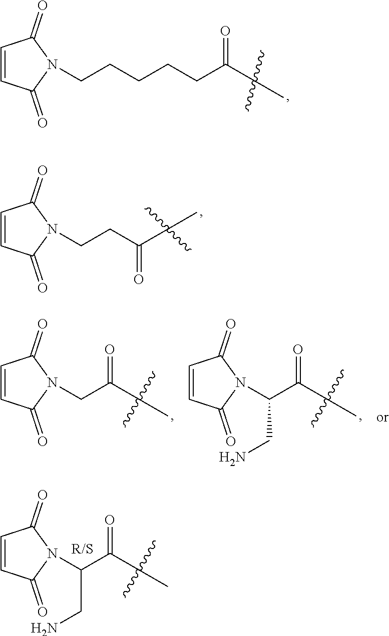

wherein the wavy lines indicate the point of covalent attachment to the rest of the Drug-Linker compound.

In some embodiments, the Connector Unit A is

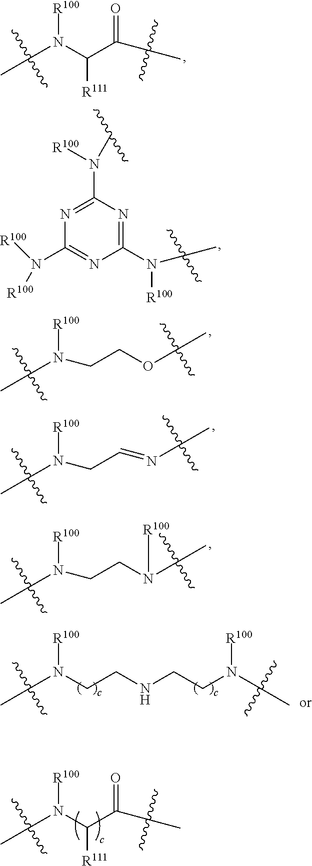

wherein
each R¹⁰⁰ is independently selected from hydrogen or —C₁-C₃ alkyl;
R¹¹¹ is independently selected from the group consisting of hydrogen, p-hydroxybenzyl, methyl, isopropyl, isobutyl, sec-butyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-,

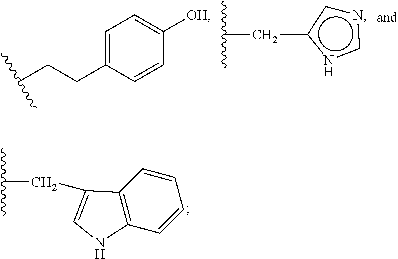

each subscript c is an independently selected integer from 1 to 10; and
the wavy lines indicate attachment of the Connector Unit to the rest of the Drug-Linker compound.

In some embodiments, the Connector Unit A is

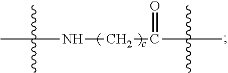 [SMILES: *NCC(*)=O]

c is an integer ranging from 1 to 6; and
the wavy lines indicate the site of attachment to the rest of the Drug-Linker compound or salt thereof.

In some embodiments, A is a bond.

In some embodiments, B is

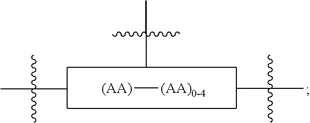

each AA is independently a proteinogenic or non-proteinogenic amino acid; and
the wavy lines indicate points of attachment to the rest of the Drug-Linker compound or salt thereof.

In some embodiments, B is an amino acid. In some embodiments, B is

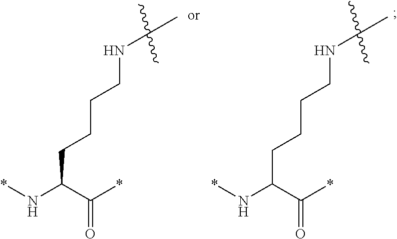 [SMILES: *NCCCC[C@H](N*)C(*)=O ; *NCCCCC(N*)C(*)=O]

the wavy line indicates the point of attachment to the Partitioning Agent S*; and
the asterisks indicate points of attachment to the rest of the Drug-Linker structure.

In some embodiments, the Partitioning Agent S* is a polyethylene glycol (PEG) unit, cyclodextrin unit, polyamide, hydrophilic peptide, polysaccharide, or dendrimer. In some embodiments, Partitioning Agent S* is a PEG Unit comprising from 4 to 72 (CH₂CH₂O) subunits. In some embodiments, the PEG Unit is

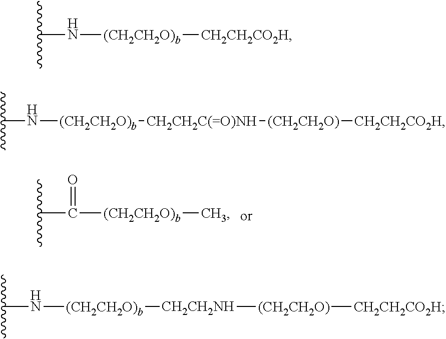

b is selected from the group consisting of 4 to 36; and
the wavy lines indicate the site of attachment to the rest of the Drug-Linker compound or salt thereof.

In some embodiments, the Releasable Linker RL is
-(AA)₁₋₁₂-; and
each AA is independently a proteinogenic or non-proteinogenic amino acid.

In some embodiments, the Releasable Linker RL is -AA₁-AA₂- or -AA₁-AA₂-AA₃-, wherein AA₁ is attached to the Stretcher Unit Z′ or the Connector Unit A. In some embodiments, the Releasable Linker RL is

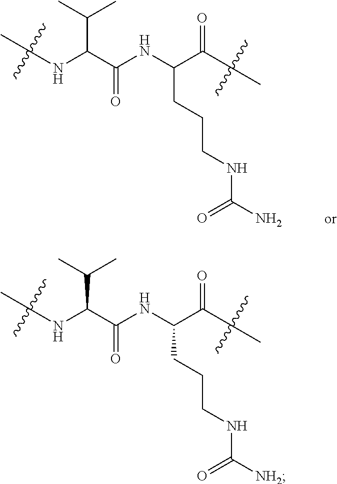 [SMILES: *NC(C(=O)NC(CCCNC(N)=O)C(*)=O)C(C)C ; *N[C@H](C(=O)N[C@@H](CCCNC(N)=O)C(*)=O)C(C)C]

and
the wavy line adjacent to the —NH— group indicates attachment to the Stretcher Unit Z′ or the Connector Unit A and the wavy line adjacent to the —C(═O)— group indicates attachment to the Spacer Unit Y or the Drug Unit D.

In some embodiments, the Releasable Linker RL is a glycoside. In some embodiments, the Releasable Linker RL is

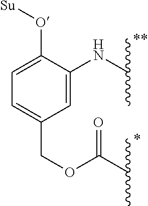

wherein
Su is a hexose form a monosaccharide;
O′ represents the oxygen atom of a glycosidic bond that is capable of cleavage by a glycosidase;
the wavy line marked with a single asterisk (*) indicates the site of covalent attachment to D; and
the wavy line marked with a double asterisk (**) indicates the site of covalent attachment to the remainder of Q.

In some embodiments, the Releasable Linker RL is

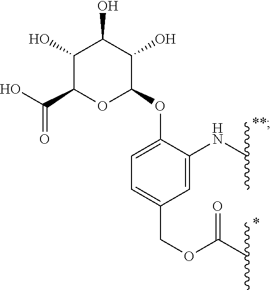

the wavy line marked with a single asterisk (*) indicates the site of covalent attachment to D; and
the wavy line marked with a double asterisk (**) indicates the site of covalent attachment to the remainder of Q.

In some embodiments, the Spacer Unit Y is

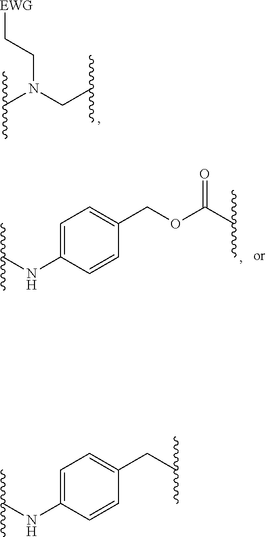

wherein EWG is an electron-withdrawing group; and
the wavy lines indicate the site of attachment to the rest of the Drug-Linker compound or salt thereof.

In some embodiments, the Spacer Unit Y is

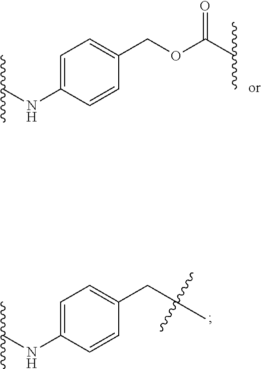 [SMILES: *Nc1ccc(COC(*)=O)cc1 ; "or" ; *Cc1ccc(N*)cc1 ; ";"]

the wavy lines indicate the site of attachment to the rest of the Drug-Linker compound or salt thereof.

In some embodiments,
Z′ is

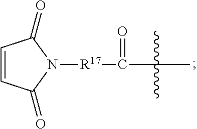

R¹⁷ is C₁-C₁₀ alkylene;
A is a bond;
RL is -AA₁-AA₂-;
AA₁ and AA₂ are each independently a proteinogenic amino acid;
Y is

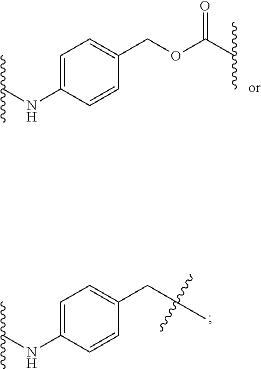 [SMILES: *Nc1ccc(COC(*)=O)cc1 ; "or" ; *Cc1ccc(N*)cc1 ; ";"]

and
the wavy lines indicate the site of attachment to the rest of the Drug-Linker compound or salt thereof.

In some embodiments,
Z′ is

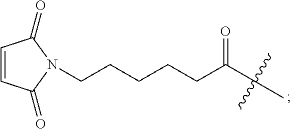 [SMILES: *C(=O)CCCCCN1C(=O)C=CC1=O ; ";"]

A is a bond;
RL is

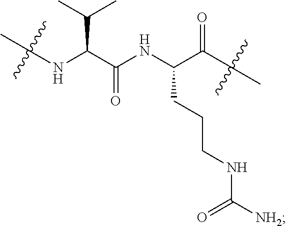 [SMILES: *N[C@H](C(=O)N[C@@H](CCCNC(N)=O)C(*)=O)C(C)C]

Y is

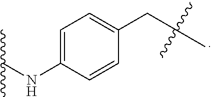 [SMILES: *Cc1ccc(N*)cc1 ; "."]

In some embodiments, Y-D is

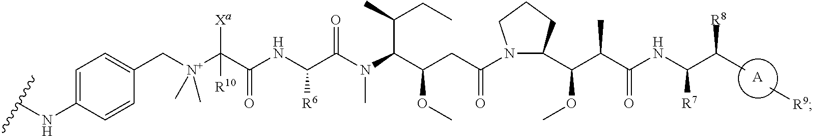

and
the wavy line indicates the site of attachment to the rest of the Drug-Linker compound or salt thereof.

In some embodiments, Y-D is

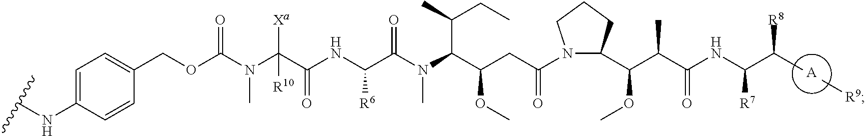

and
the wavy line indicates the site of attachment to the rest of the Drug-Linker compound or salt thereof.

In some embodiments, the compound is

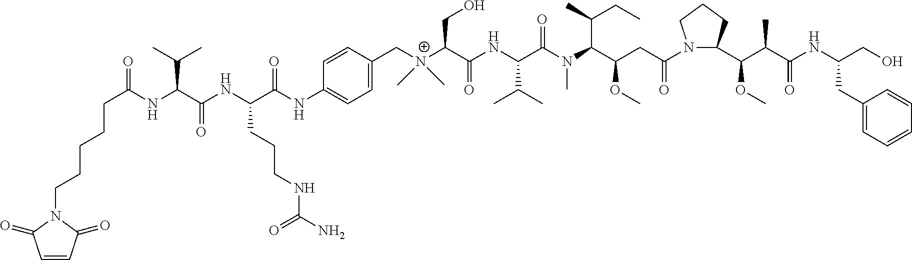 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)[N+](C)(C)Cc1ccc(NC(=O)[C@H](CCCNC(N)=O)NC(=O)[C@@H](NC(=O)CCCCCN2C(=O)C=CC2=O)C(C)C)cc1)C(C)C]

or a salt thereof.

In some embodiments, the compound is

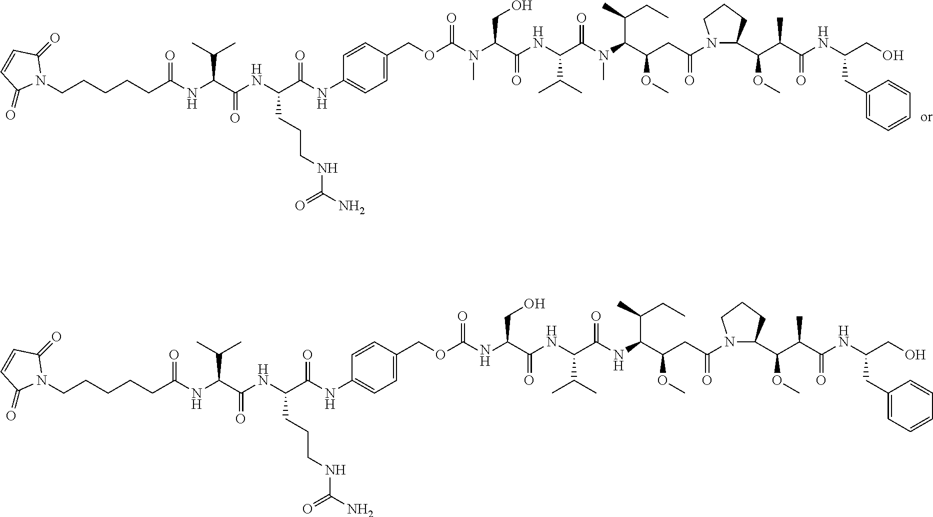 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C ; "or" ; CC[C@H](C)[C@H](NC(=O)[C@@H](NC(=O)[C@H](CO)NC(=O)OCc1ccc(NC(=O)[C@H](CCCNC(N)=O)NC(=O)[C@@H](NC(=O)CCCCCN2C(=O)C=CC2=O)C(C)C)cc1)C(C)C)[C@@H](CC(=O)N1CCC[C@H]1[C@H](OC)[C@@H](C)C(=O)N[C@H](CO)Cc1ccccc1)OC]

or a salt thereof.

In some embodiments, the Drug-Linker compound is a compound of Table 2.

Also provided herein is a Ligand-Drug Conjugate compound of the formula:

L-(Q-D)ₚ

or a pharmaceutically acceptable salt thereof, wherein
L is a Ligand Unit;
Q is a Linker Unit selected from the group consisting of:
   (i) Z′-A-RL-,
   (ii) Z′-A-RL-Y—,
   (iii) Z′-A-S*-RL-,
   (iv) Z′-A-S*-RL-Y—,
   (v) Z′-A-B(S*)—RL-,
   (vi) Z′-A-B(S*)-RL-Y—,
   (vii) Z′-A-,
   (viii) Z′-A-S*—W—,
   (ix) Z′-A-B(S*)—W—,
   (x) Z′-A-S*—W-RL-, and
   (xi) Z′-A-B(S*)—W-RL-;
Z′ is a Stretcher Unit precursor;
A is a bond or a Connector Unit;
B is a Parallel Connector Unit;
S* is a Partitioning Agent;
RL is a Releasable Linker;
W is a Amino Acid Unit;
Y is a Spacer Unit; and
D is a Drug Unit of Formula (I′):

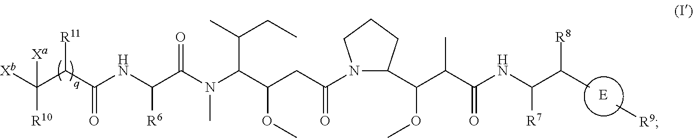

wherein
X^{b} is —NR²—^{#} or —N⁺R¹R⁵—^{#}, wherein the # denotes the point of attachment to Q, and X^{a} is

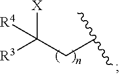

or
X^{a} and X^{b} are taken together with the carbon atom to which they are attached to form

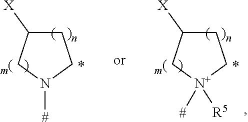

wherein the asterisk denotes the carbon atom of Formula (I) that bears the X^{a} and X^{b} groups, and the # denotes the point of attachment to Q;
R¹ and R⁵ are independently C₁-C₄ alkyl;
X is H, OH, —C(O)NR^{a}R^{b}, —S(O)₂R^{a}, —S(O)—R^{a} —S(O)₂NR^{a}R^{b}, —NHS(O)₂R^{a}, or —NHC(O)R^{a};
R², R³, R⁴, R¹⁰, R^{a}, and R^{b} are each independently H or C₁-C₄ alkyl;
R⁶ is C₁-C₄ alkyl optionally substituted with OH;
R⁷ is H, C₁-C₄ alkyl optionally substituted with one or two OH moieties, or 5-6 membered heteroaryl;
E is phenyl or 5-6 membered heteroaryl;
R⁸, R⁹, and R¹¹ are each independently H or OH;
n is 0, 1, 2, or 3;
m is 1, 2, 3, or 4;
q is 0 or 1; and
wherein when X is H, at least two of R⁷, R⁸, R⁹, and R¹¹ comprise an OH moiety.

In some embodiments, the Linker Unit Q is of formula (i), (ii), (iii), (iv), (x), or (xi). In some embodiments, the Linker Unit Q is of formula (v), (vi), (ix), or (xi). In some embodiments, the Linker Unit Q is of formula (viii), (ix), (x), or (xi).

In some embodiments, the Ligand Unit L and the Stretcher Unit Z together are

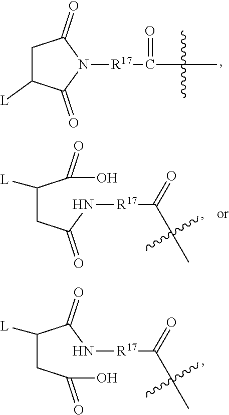

wherein
R¹⁷ is —CH₂CH₂(OCH₂CH₂)ₖ—, —C₁-C₁₀ alkylene-, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—;
subscript k is an integer ranging from 1 to 36;
R¹⁷ is optionally substituted by a Basic Unit (BU) such as an aminoalkyl moiety, e.g. —(CH₂)ₓNH₂, —(CH₂)ₓNHR^{a}, and —(CH₂)ₓNR^{a}₂, wherein x is an integer of from 1-4 and each R^{a} is independently selected from the group consisting of C₁₋₆ alkyl and C₁₋₆ haloalkyl, or two R^{a} groups are combined with the nitrogen to which they are attached to form an azetidinyl, pyrrolidinyl or piperidinyl group; and
the wavy line indicates the point of covalent attachment to the rest of the Ligand-Drug Conjugate compound.

In some embodiments, the Ligand Unit L and the Stretcher Unit Z together are

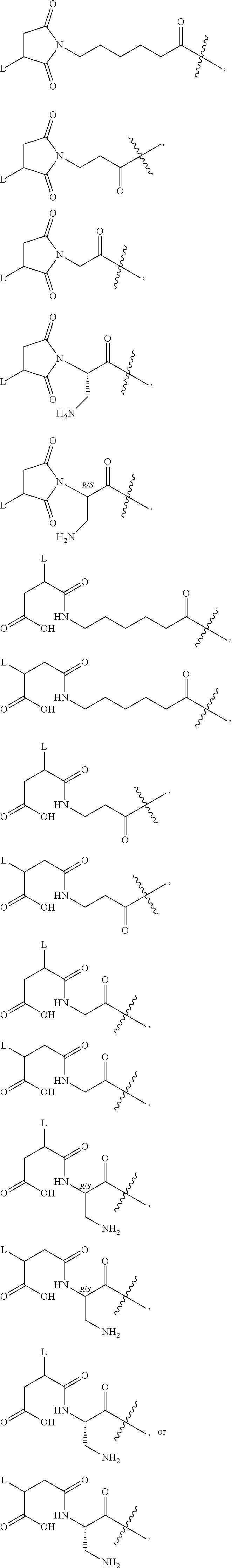

wherein the wavy lines indicate the point of covalent attachment to the rest of the Ligand-Drug Conjugate compound.

In some embodiments, the Connector Unit A is

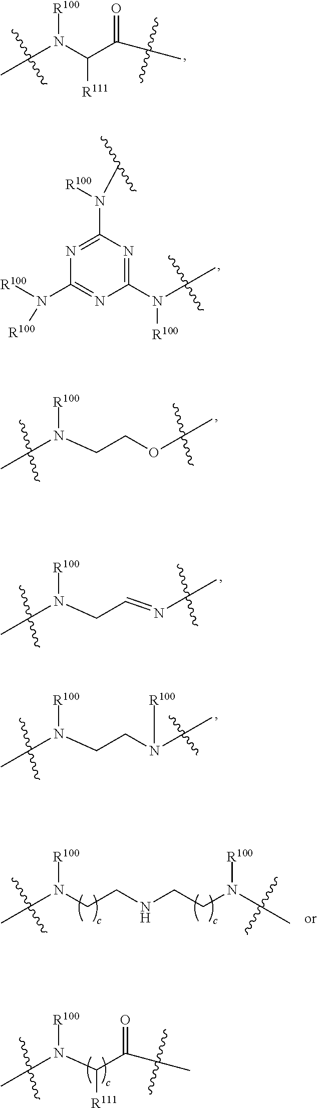

wherein
each R¹⁰⁰ is independently selected from hydrogen or —C₁-C₃ alkyl;
R¹¹¹ is independently selected from the group consisting of hydrogen, p-hydroxybenzyl, methyl, isopropyl, isobutyl, sec-butyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-,

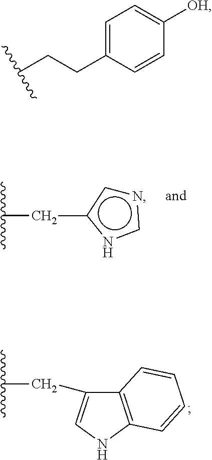

each subscript c is an independently selected integer from 1 to 10; and
the wavy lines indicate attachment of the Connector Unit to the rest of the Ligand-Drug Conjugate compound or pharmaceutically acceptable salt thereof.

In some embodiments, the Connector Unit A is

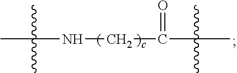

c is an integer ranging from 1 to 6; and
the wavy lines indicate the site of attachment to the rest of the Ligand-Drug Conjugate compound or pharmaceutically acceptable salt thereof.

In some embodiments, A is a bond.

In some embodiments, B is

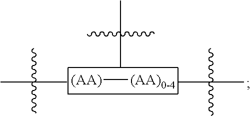

each AA is independently a proteinogenic or non-proteinogenic amino acid; and
the wavy lines indicate points of attachment to the rest of the Ligand-Drug Conjugate compound or pharmaceutically acceptable salt thereof.

In some embodiments, B is an amino acid. In some embodiments, B is

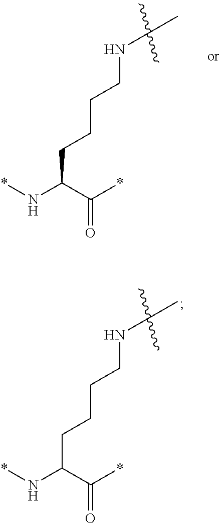 [SMILES: *NCCCC[C@H](N*)C(*)=O ; *NCCCCC(N*)C(*)=O]

the wavy line indicates the point of attachment to the Partitioning Agent S*; and
the asterisks indicate points of attachment to the rest of the Ligand-Drug Conjugate compound or pharmaceutically acceptable salt thereof.

In some embodiments, the Partitioning Agent S* is a polyethylene glycol (PEG) unit, cyclodextrin unit, polyamide, hydrophilic peptide, polysaccharide, or dendrimer. In some embodiments, the Partitioning Agent S* is a PEG Unit comprising from 4 to 72 (CH₂CH₂O) subunits. In some embodiments, the PEG Unit is

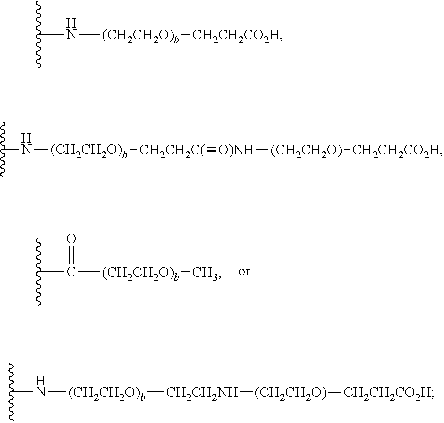

b is selected from the group consisting of 4 to 36; and
the wavy lines indicate the site of attachment to the rest of the Ligand-Drug Conjugate compound or pharmaceutically acceptable salt thereof.

In some embodiments, the Releasable Linker RL is
-(AA)₁₋₁₂-; and
each AA is independently a proteinogenic or non-proteinogenic amino acid.

In some embodiments, the Releasable Linker RL is -AA₁-AA₂- or -AA₁-AA₂-AA₃-, wherein AA₁ is attached to the Stretcher Unit Z or the Connector Unit A.

In some embodiments, the Releasable Linker RL is

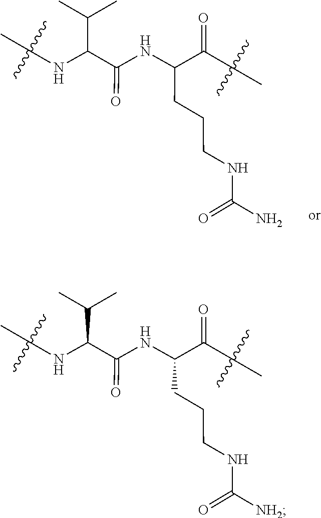 [SMILES: *NC(C(=O)NC(CCCNC(N)=O)C(*)=O)C(C)C ; *N[C@H](C(=O)N[C@@H](CCCNC(N)=O)C(*)=O)C(C)C]

and
the wavy line adjacent to the —NH— group indicates attachment to the Stretcher Unit Z or the Connector Unit A and the wavy line adjacent to the —C(═O)— group indicates attachment to the Spacer Unit Y or the Drug Unit D.

In some embodiments, the Releasable Linker RL is a glycoside.

In some embodiments, the Releasable Linker RL is

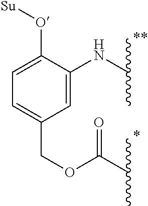

wherein
Su is a hexose form a monosaccharide;
O′ represents the oxygen atom of a glycosidic bond that is capable of cleavage by a glycosidase;
the wavy line marked with a single asterisk (*) indicates the site of covalent attachment to D; and
the wavy line marked with a double asterisk (**) indicates the site of covalent attachment to the remainder of Q.

In some embodiments, the Releasable Linker RL is

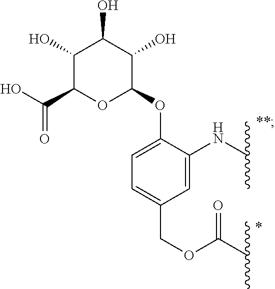 [SMILES: *Nc1cc(COC(*)=O)ccc1O[C@H]1O[C@@H](C(=O)O)[C@H](O)[C@@H](O)[C@@H]1O]

the wavy line marked with a single asterisk (*) indicates the site of covalent attachment to D; and
the wavy line marked with a double asterisk (**) indicates the site of covalent attachment to the remainder of Q.

In some embodiments, the Spacer Unit Y is

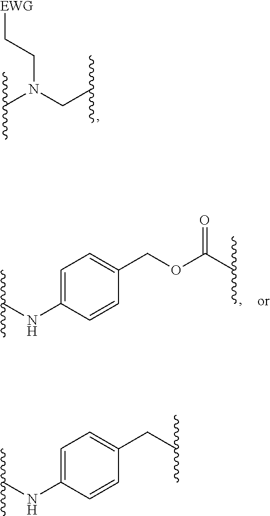

wherein EWG is an electron-withdrawing group; and
the wavy lines indicate the site of attachment to the rest of the Ligand-Drug Conjugate compound or pharmaceutically acceptable salt thereof.

In some embodiments, the Spacer Unit Y is

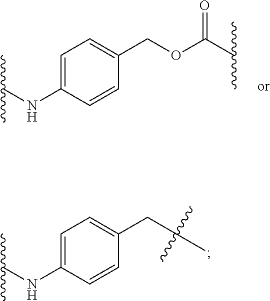 [SMILES: *Nc1ccc(COC(*)=O)cc1 ; *Cc1ccc(N*)cc1]

and
the wavy lines indicate the site of attachment to the rest of the Ligand-Drug Conjugate compound or pharmaceutically acceptable salt thereof.

In some embodiments,
R¹⁷ is C₁-C₁₀ alkylene;
A is a bond;
RL is -AA₁-AA₂-;
AA₁ and AA₂ are each independently a proteinogenic amino acid;

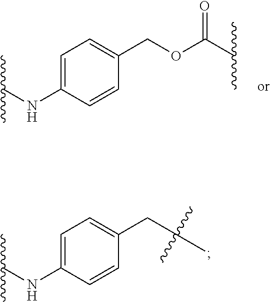 [SMILES: *Nc1ccc(COC(*)=O)cc1 ; *Cc1ccc(N*)cc1]

and
the wavy lines indicate the site of attachment to the rest of the Ligand-Drug Conjugate compound or pharmaceutically acceptable salt thereof.

In some embodiments,
A is a bond;
RL is

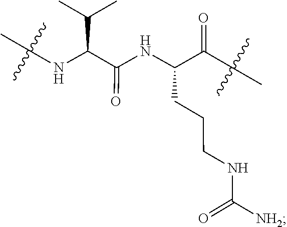 [SMILES: *N[C@H](C(=O)N[C@@H](CCCNC(N)=O)C(*)=O)C(C)C]

and

Y is

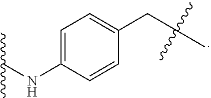 [SMILES: *Cc1ccc(N*)cc1]

In some embodiments, Y-D is

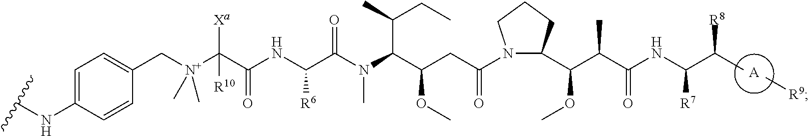

and
the wavy line indicates the site of attachment to the rest of the Ligand-Drug Conjugate compound or pharmaceutically acceptable salt thereof.

In some embodiments, Y-D is

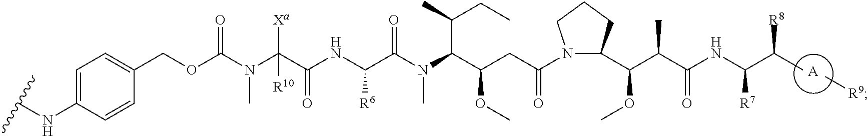

and
the wavy line indicates the site of attachment to the rest of the Ligand-Drug Conjugate compound or pharmaceutically acceptable salt thereof.

In some embodiments, the compound is

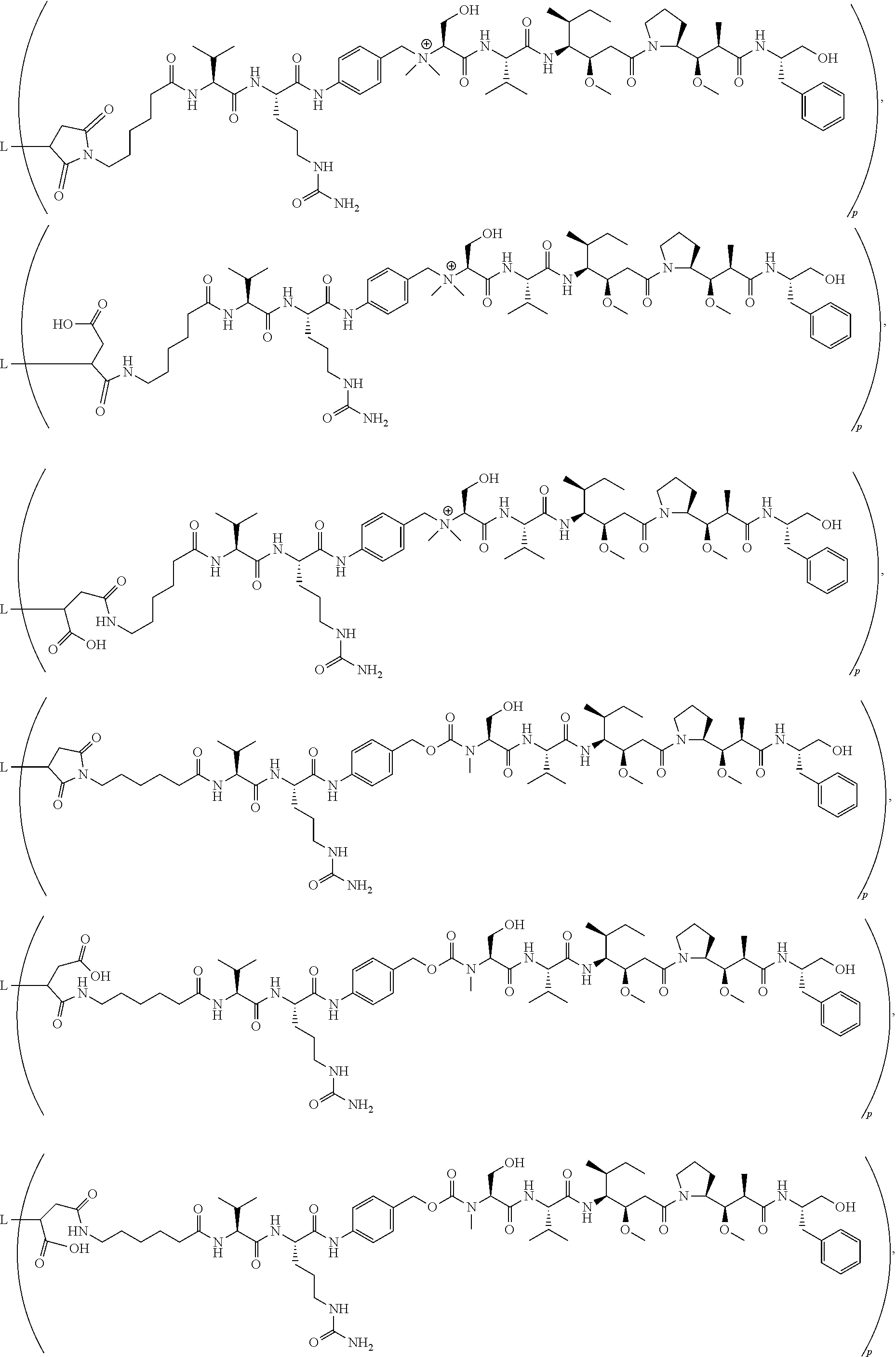

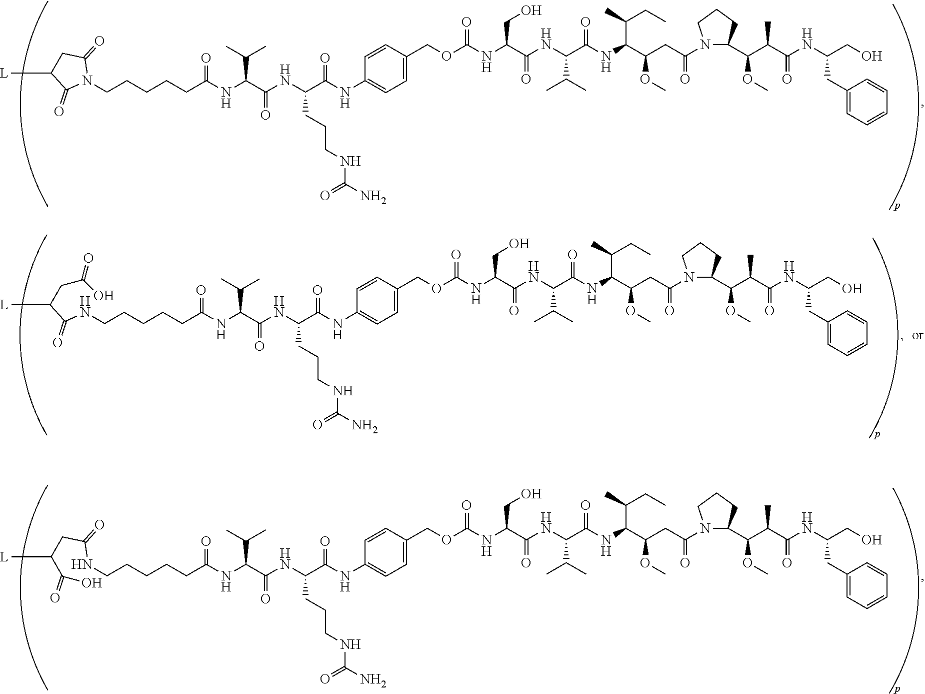

or a pharmaceutically acceptable salt thereof.

In some embodiments, the Ligand-Drug Conjugate compound is a compound of Table 3.

In some embodiments, p is an integer ranging from 2 to 6. In some embodiments, p is 4.

Also provided herein is a pharmaceutical composition comprising a Ligand-Drug Conjugate compound as described herein and a pharmaceutically acceptable excipient. In some embodiments, the composition comprises a plurality of Ligand-Drug Conjugate compounds with an average drug loading from 2 to 8. In some embodiments, the average drug loading is about 4. In some embodiments, the average drug loading is from 3.5 to 4.5.

Also provided herein is a method of treating cancer comprising administering a therapeutically effective amount of the Ligand-Drug Conjugate compound of any one of claims 66-96, or a pharmaceutically acceptable salt thereof, to a subject in need thereof. In some embodiments, the subject tolerates treatment with the Ligand-Drug Conjugate compound better than treatment with another Ligand-Drug Conjugate compound in therapeutically effective doses. In some embodiments, the another Ligand-Drug Conjugate compound comprises a monomethyl auristatin E or monomethyl auristatin F Drug Unit.

### BRIEF DESCRIPTION OF THE FIGURES

FIG. 1. Illustrates the in vivo mean tumor volume data for various Ag1 ADCs in the A2058 melanoma xenograft model.

FIG. 2. Illustrates the in vivo mean tumor volume data for various h2A2 ADCs in the Detroit562 pharyngeal cancer xenograft model.

FIG. 3. Illustrates the in vivo median tumor volume data for various cAC10 ADCs in the Karpas/KarpasBVR admixed Hodgkin lymphoma xenograft model.

FIG. 4. Illustrates the in vivo mean tumor volume data for various cAC10 ADCs in the Karpas Hodgkin lymphoma xenograft model.

FIG. 5. Illustrates the in vivo mean tumor volume data for various cAC10 ADCs in the Karpas Hodgkin lymphoma xenograft model.

FIG. 6. Summarizes plasma neutrophil levels at day 5 and day 8 following administration by non-binding ADCs to rats.

FIG. 7. Summarizes reticulocyte levels at day 5 and day 8 following administration by non-binding ADCs to rats.

FIG. 8. Summarizes platelet levels at day 5 and day 8 following administration by non-binding ADCs to rats.

FIG. 9. Summarizes aspartate transaminase (AST) levels at day 8 following administration by non-binding ADCs to rats.

FIG. 10. Illustrates the efficacy of a variety of hCR011 ADCs and a non-binding control ADC as measured in the WM2664 melanoma xenograft model.

FIG. 11. Illustrates the efficacy of a variety of hCR011 ADCs and non-binding control ADCs as measured in the SKMEL5 melanoma xenograft model.

FIG. 12. Illustrates the efficacy of non-binding, Ag2, and hCR011 ADCs as measured in the PDX_1 melanoma xenograft model.

FIG. 13. Illustrates the efficacy of non-binding, Ag2 and hCR011 ADCs as measured in the PDX_4 melanoma xenograft model.

FIG. 14. Illustrates the efficacy of non-binding, Ag2 and hCR011 ADCs as measured in the PDX_3 NSCLC xenograft model.

FIG. 15. Illustrates the efficacy of non-binding, Ag2 and hCR011 ADCs as measured in the PDX_2 NSCLC xenograft model.

FIG. 16. Summarizes the anti-tumor activity of 6 hCR011 ADCs using AUC.3.

FIG. 17. Summarizes plasma neutrophil levels at baseline, day 4, day 15, and day 29 following administration of hCR011 ADCs to cynomologous monkeys.

FIG. 18. Summarizes reticulocyte levels at baseline, day 4, day 15, and day 29 following administration of hCR011 ADCs to cynomologous monkeys.

FIG. 19. Summarizes platelet levels at baseline, day 4, day 15, and day 29 following administration of hCR011 ADCs to cynomologous monkeys.

FIG. 20. Summarizes aspartate transaminase levels at baseline and day 8 following administration of hCR011 ADCs to cynomologous monkeys.

FIG. 21. shows neutrophil counts from blood samples taken prior to dosing and post-dose on Day 8.

### DETAILED DESCRIPTION OF THE INVENTION

One drug class of interest for use in Ligand-Drug Conjugates is auristatins. Auristatins have been shown to be effective payloads in some Ligand-Drug Conjugates (LDCs), but it is believed that their hydrophobicity can contribute to off-target toxicity due to increased permeability and high bystander activity of the free auristatin. Additionally, hydrophobic payloads, such as auristatins, may increase the hydrophobicity of the LDC, resulting in rapid clearance of the LDC from the body of a subject. Therefore, there is a need for auristatin conjugates engineered to have optimized cell permeability, pharmacokinetics, and toxicity profile.

Without being bound by theory, it is believed that the hydrophilic groups of the auristatin compounds provided herein influence the properties of the compounds and of resulting conjugates (e.g., Ligand-Drug Conjugates). The hydrophilic groups are believed to increase the effectiveness of the resulting conjugates in two ways. Conjugates comprising a hydrophilic auristatin group may improve tumor exposure to the Drug Unit due to decreased plasma clearance, and they may simultaneously demonstrate reduced off-target toxicity due to decreased cell permeability of the free drug after it is released from the conjugate.

### I. Definitions

Unless stated otherwise, the following terms and phrases as used herein are intended to have the following meanings. When trade names are used herein, the trade name includes the product formulation, the generic drug, and the active pharmaceutical ingredient(s) of the trade name product, unless otherwise indicated by context.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5^{th} ed., 2013, Academic Press; and the Oxford Dictionary Of Biochemistry And Molecular Biology, 2^{nd} ed., 2006, Oxford University Press, provide one of skill with a general dictionary of many of the terms used in this disclosure.

Unless otherwise required by context or expressly indicated, singular terms shall include pluralities and plural terms shall include the singular.

It is understood that aspect and embodiments of the invention described herein include “comprising,” “consisting,” and/or “consisting essentially of” aspects and embodiments.

As used herein, the singular form “a”, “an”, and “the” should be understood to refer to “one or more” of any recited or enumerated component unless indicated otherwise.

The term “about” refers to a value or composition that is within an acceptable error range for the particular value or composition as determined by one of ordinary skill in the art, which will depend in part on how the value or composition is measured or determined, i.e., the limitations of the measurement system. As is understood by one skilled in the art, reference to “about” a value or parameter herein includes (and describes) embodiments that are directed to that value or parameter per se. For example, description referring to “about X” includes description of “X”.

As described herein, any concentration range, percentage range, ratio range, or integer range is to be understood to include the value of any integer within the recited range and, when appropriate, fractions thereof (such as one tenth and one hundredth of an integer), unless otherwise indicated.

When a trade name is used herein, reference to the trade name also refers to the product formulation, the generic drug, and the active pharmaceutical ingredient(s) of the trade name product, unless otherwise indicated by context.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “antibody” as used herein is used in the broadest sense and specifically covers intact monoclonal antibodies, polyclonal antibodies, monospecific antibodies, multispecific antibodies (e.g., bispecific antibodies), and antibody fragments (i.e., antigen-binding fragments) that retain, at least in part, one or more of the desired biological activities of the full-length antibody, for example, affinity for its cognate antigen. The native form of an antibody is a tetramer and consists of two identical pairs of immunoglobulin chains, each pair having one light chain and one heavy chain. In each pair, the light and heavy chain variable regions (V_{L} and V_{H}) are together primarily responsible for binding to an antigen. The light chain and heavy chain variable domains consist of a framework region interrupted by three hypervariable regions, also called “complementarity determining regions” or “CDRs.” In some aspects, the constant regions are recognized by and interact with the immune system (see, e.g., Janeway et al., 2001, Immunol. Biology, 5th Ed., Garland Publishing, New York). The antibodies herein are of any type (e.g., IgG, IgE, IgM, IgD, and IgA), class (e.g., IgG₁, IgG₂, IgG₃, IgG₄, IgA₁ and IgA₂), or subclass thereof. In some aspects, the antibody is derived from a suitable species. In some embodiments, the antibody is of human or murine origin. In some aspects, the antibody is human, humanized, or chimeric. An antibody can be human, humanized, chimeric and/or affinity matured, as well as an antibody from other species, for example, mouse and rabbit, etc. The term “antibody” thus includes, for instance, a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). The term “antibody” also includes, but is not limited to, synthetic antibodies, recombinantly produced antibodies, camelized antibodies, intrabodies, anti-idiotypic (anti-Id) antibodies, and functional fragments (e.g., antigen-binding fragments) of any of the above, which refers to a portion of an antibody heavy and/or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabodies, tetrabodies, and minibodies. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Pluckthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule.

The term “monoclonal antibody” as used herein refers to an antibody obtained from a population of substantially homogeneous antibodies, i.e., the individual antibodies comprising the population are identical except for possible naturally occurring mutations that are present, in some aspects, in minor amounts. Monoclonal antibodies are highly specific, being directed against a single antigenic site. The modifier “monoclonal” indicates the character of the antibody as being obtained from a substantially homogeneous population of antibodies and is not to be construed as requiring production of the antibody by any particular method.

An “intact antibody” is one which comprises an antigen-binding variable region as well as a light chain constant domain (C_{L}) and heavy chain constant domains, C_{H}1, C_{H}2, C_{H}3, and C_{H}4, as appropriate for the antibody class. In some aspects the constant domains are native sequence constant domains (e.g., human native sequence constant domains) and in other aspects are amino acid sequence variants thereof.

An “antibody fragment” comprises a portion of an intact antibody, comprising the antigen-binding or variable region thereof. Examples of antibody fragments include Fab, Fab′, F(ab′)₂, and Fv fragments, diabodies, triabodies, tetrabodies, linear antibodies, single-chain antibody molecules, scFv, scFv-Fc, multispecific antibody fragments formed from antibody fragment(s), a fragment(s) produced by a Fab expression library, or an epitope-binding fragments of any of the above which immunospecifically bind to a target antigen (e.g., a cancer cell antigen, a viral antigen or a microbial antigen).

An “antigen-binding fragment” (or simply “fragment”) or “antigen-binding domain”, of an antigen binding protein (e.g., an antibody) as used herein refers to one or more fragments of an antigen binding protein (e.g., an antibody), regardless of how obtained or synthesized, that retain the ability to specifically bind to the antigen bound by the whole antigen binding protein. Examples of antibody fragments include, but are not limited to, Fv; Fab; Fab′; Fab′-SH; F(ab′)₂; diabodies; linear antibodies; single-chain antibody molecules (e.g. scFv); and multispecific antibodies formed from antibody fragments. A “Fv” fragment includes a non-covalently-linked dimer of one heavy chain variable domain and one light chain variable domain. A “Fab” fragment includes, the constant domain of the light chain and the first constant domain (C_{H1}) of the heavy chain, in addition to the heavy and light chain variable domains of the Fv fragment. A “F(ab′)₂” fragment includes two Fab fragments joined, near the hinge region, by disulfide bonds.

An “antigen” is an entity to which an antibody specifically binds.

The terms “specific binding” and “specifically binds” mean that the antibody or antibody derivative will bind, in a highly selective manner, with its corresponding epitope of a target antigen and not with the multitude of other antigens. Typically, the antibody or antibody derivative binds with an affinity of at least about 1×10⁻⁷ M, and preferably 10⁻⁸ M to 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely-related antigen.

The term “inhibits” or “inhibition of” means to reduce by a measurable amount, or to prevent entirely.

The term “therapeutically effective amount” refers to an amount of a conjugate effective to treat a disease or disorder in a mammal. In the case of cancer, the therapeutically effective amount of the conjugate may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. In some aspects, the drug inhibits growth and/or kills existing cancer cell. In some aspects, the drug is cytostatic and/or cytotoxic. For cancer therapy, efficacy is measured by commonly available tools. In some aspects, efficacy is measured by assessing the time to disease progression (TTP) and/or determining the response rate (RR).

As used herein, “Percent (%) amino acid sequence identity” and “homology” with respect to a peptide, polypeptide or antigen binding protein (e.g., antibody) sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared. For example, the % sequence identity of a given amino acid sequence A to, with, or against a given amino acid sequence B (which can alternatively be phrased as a given amino acid sequence A that has or comprises a certain % sequence identity to, with, or against a given amino acid sequence B) is calculated as follows:

100 times the fraction X/Y

where X is the number of amino acid residues scored as identical matches by the sequence in that program's alignment of A and B, and where Y is the total number of amino acid residues in B. Unless specifically stated otherwise, all % amino acid sequence identity values used herein are calculated according to this formula using the ALIGN-2 computer program. It will be appreciated that where the length of amino acid sequence A is not equal to the length of amino acid sequence B, the % sequence identity of A to B will not equal the % sequence identity of B to A.

The term “substantial” or “substantially” refers to a majority, i.e. >50% of a population, of a mixture or a sample, preferably more than 50%, 55%, 60%, 65%, 70%, 75%, 80%, 85%, 90%, 91%, 92%, 93%, 94%, 95%, 96%, 97%, 98%, or 99% of a population.

The term “cytotoxic activity” refers to a cell-killing effect of a drug or Ligand-Drug Conjugate compound or an intracellular metabolite of a Ligand-Drug Conjugate compound. In some aspects, cytotoxic activity is expressed as the IC₅₀ value, which is the concentration (molar or mass) per unit volume at which half the cells survive.

The term “cytostatic activity” refers to an anti-proliferative effect of a drug or Ligand-Drug Conjugate compound or an intracellular metabolite of a Ligand-Drug Conjugate compound.

The term “cytotoxic agent” as used herein refers to a substance that has cytotoxic activity and causes destruction of cells. The term is intended to include chemotherapeutic agents, and toxins such as small molecule toxins or enzymatically active toxins of bacterial, fungal, plant or animal origin, including synthetic analogs and derivatives thereof.

The term “cytostatic agent” as used herein refers to a substance that inhibits a function of cells, including cell growth or multiplication. Cytostatic agents include inhibitors such as protein inhibitors, e.g., enzyme inhibitors. Cytostatic agents have cytostatic activity.

The terms “cancer” and “cancerous” refer to or describe the physiological condition or disorder in mammals that is typically characterized by unregulated cell growth. A “tumor” comprises one or more cancerous cells.

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See, for instance, Fundamental Immunology (Paul, W., ed., 7^{th} ed. Raven Press, N.Y. (2013)). Briefly, each heavy chain typically is comprised of a heavy chain variable region (abbreviated herein as V_{H} or VH) and a heavy chain constant region (C_{H} or CH). The heavy chain constant region typically is comprised of three domains, C_{H}1, C_{H}2, and C_{H}3. The heavy chains are generally inter-connected via disulfide bonds in the so-called “hinge region.” Each light chain typically is comprised of a light chain variable region (abbreviated herein as V_{L} or VL) and a light chain constant region (C_{L} or CL). The light chain constant region typically is comprised of one domain, C_{L}. The CL can be of κ (kappa) or λ (lambda) isotype. The terms “constant domain” and “constant region” are used interchangeably herein. An immunoglobulin can derive from any of the commonly known isotypes, including but not limited to IgA, secretory IgA, IgG, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes.

The term “hypervariable region” or “HVR,” as used herein, refers to each of the regions of an antibody variable domain which are hypervariable in sequence. HVRs can form structurally defined loops (“hypervariable loops”). Generally, native four-chain antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N J, 2003). Indeed, naturally-occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

HVRs generally comprise amino acid residues from the hypervariable loops and/or from the “complementary determining regions” (CDRs), CDRs being of highest sequence variability and/or involved in antigen recognition. A variety of schemes for defining the boundaries of a given CDR are known in the art. For example, the Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, MD. (1991)). Chothia refers instead to the location of the structural loops (Chothia and Lesk, J. Mol. Biol. 196:901-917 (1987)). The AbM CDRs represent a compromise between the Kabat CDRs and Chothia structural loops and are used by Oxford Molecular's AbM antibody modeling software. The “contact” CDRs are based on an analysis of the available complex crystal structures. Additional details on the foregoing schemes as well as other numbering conventions are provided in the following references: Al-Lazikani et al., (1997) J. Mol. Biol. 273: 927-948 (“Chothia” numbering scheme); MacCallum et al., (1996) J. Mol. Biol. 262:732-745 (1996), (Contact” numbering scheme); Lefranc M-P., et al., (2003) Dev. Comp. Immunol. 27:55-77 (“IMGT” numbering scheme); and Honegger A. & Pluckthun A. (2001) J. Mol/Biol. 309:657-70, (AHo numbering scheme).

In some embodiments, the HVR regions and associated sequences are the same as the CDR regions and associated sequences based upon one of the foregoing numbering conventions. As such, residues for exemplary HVRs and/or CDRs are summarized in the Table D1 below.

TABLE D1
Summary of Different CDR Numbering Schemes.

| Loop | IMGT | Kabat | AbM | Chothia | Contact |
|---|---|---|---|---|---|
| CDR-H1 | 27-38 | 31-35 | 26-35 | 26-32 | 30-35 |
| CDR-H2 | 56-65 | 50-65 | 50-58 | 52-56 | 47-58 |
| CDR-H3 | 105-117 | 95-102 | 95-102 | 95-102 | 93-101 |
| CDR-L1 | 27-38 | 24-34 | 24-34 | 24-34 | 30-36 |
| CDR-L2 | 56-65 | 50-56 | 50-56 | 50-56 | 46-55 |
| CDR-L3 | 105-117 | 89-97 | 89-97 | 89-97 | 89-96 |

In some embodiments, HVRs can comprise extended HVRs as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

Unless otherwise specified, the terms “CDR” and “complementary determining region” of a given antibody or region thereof, such as a variable region, as well as individual CDRs (e.g., “CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, AbM, Chothia, or Contact method. In other instances, the particular amino acid sequence of a CDR is given.

Thus, in some embodiments, the antigen binding protein comprises CDRs and/or HVRs as defined by the IMGT system. In other embodiments, the antigen binding protein comprises CDRs or HVRs as defined by the Kabat system. In still other embodiments, the antigen binding protein comprises CDRs or HVRs as defined by the AbM system. In further embodiments, the antigen binding protein comprises CDRs or HVRs as defined by the Chothia system. In yet other embodiments, the antigen binding protein comprises CDRs or HVRs as defined by the IMGT system.

The term “variable region” or “variable domain” refers to the domain of an antigen binding protein (e.g., an antibody) heavy or light chain that is involved in binding the antigen binding protein (e.g., antibody) to antigen. The variable regions or domains of the heavy chain and light chain (VH and VL, respectively) of an antigen binding protein such as an antibody can be further subdivided into regions of hypervariability (or hypervariable regions, which may be hypervariable in sequence and/or form of structurally defined loops), such as hypervariable regions (HVRs) or complementarity-determining regions (CDRs), interspersed with regions that are more conserved, termed framework regions (FRs). In general, there are three HVRs (HVR-H1, HVR-H2, HVR-H3) or CDRs (CDR-H1, CDR-H2, CDR-H3) in each heavy chain variable region, and three HVRs (HVR-L1, HVR-L2, HVR-L3) or CDRs in (CDR-L1, CDR-L2, CDR-L3) in each light chain variable region. “Framework regions” and “FR” are known in the art to refer to the non-HVR or non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). Within each VH and VL, three HVRs or CDRs and four FRs are typically arranged from amino-terminus to carboxy-terminus in the following order: FR1, HVR1, FR2, HVR2, FR3, HVR3, FR4 in the case of HVRs, or FR1, CDR1, FR2, CDR2, FR3, CDR3, FR4 in the case of CDRs (See also Chothia and Lesk J. Mot. Biol., 195, 901-917 (1987)). A single VH or VL domain can be sufficient to confer antigen-binding specificity. In addition, antibodies that bind a particular antigen can be isolated using a VH or VL domain from an antibody that binds the antigen to screen a library of complementary VL or VH domains, respectively. See, e.g., Portolano et al. J. Immunol. 150:880-887 (1993); Clarkson et al., Nature 352:624-628 (1991).

The term “heavy chain variable region” (VH) as used herein refers to a region comprising heavy chain HVR-H1, FR-H2, HVR-H2, FR-H3, and HVR-H3. For example, a heavy chain variable region may comprise heavy chain CDR-H1, FR-H2, CDR-H2, FR-H3, and CDR-H3. In some embodiments, a heavy chain variable region also comprises at least a portion of an FR-H1 and/or at least a portion of an FR-H4.

The term “heavy chain constant region” as used herein refers to a region comprising at least three heavy chain constant domains, C_{H}1, C_{H}2, and C_{H}3. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an ε constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

The term “heavy chain” (HC) as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain variable region” (VL) as used herein refers to a region comprising light chain HVR-L1, FR-L2, HVR-L2, FR-L3, and HVR-L3. In some embodiments, the light chain variable region comprises light chain CDR-L1, FR-L2, CDR-L2, FR-L3, and CDR-L3. In some embodiments, a light chain variable region also comprises an FR-L1 and/or an FR-L4.

The term “light chain constant region” as used herein refers to a region comprising a light chain constant domain, C_{L}. Nonlimiting exemplary light chain constant regions include λ and κ.

The term “light chain” (LC) as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

The “EU numbering system” or “EU index” is generally used when referring to a residue in an immunoglobulin heavy chain constant region (e.g., the EU index reported in Kabat et al., Sequences of Proteins of Immunological Interest, 5th Ed. Public Health Service, National Institutes of Health, Bethesda, Md., 1991). The “EU index as in Kabat” refers to the residue numbering of the human IgG1 EU antibody. Unless stated otherwise herein, references to residue numbers in the constant domain of antibodies means residue numbering by the EU numbering system.

The term “derivative” refers to a molecule (e.g., an antigen binding protein such as an antibody or fragment thereof) that includes a chemical modification other than an insertion, deletion, or substitution of amino acids (or nucleic acids). In certain embodiments, derivatives comprise covalent modifications, including, but not limited to, chemical bonding with polymers, lipids, or other organic or inorganic moieties. In certain embodiments, a derivative of a particular antigen binding protein can have a greater circulating half-life than an antigen binding protein that is not chemically modified. In certain embodiments, a derivative can have improved targeting capacity for desired cells, tissues, and/or organs. In some embodiments, a derivative of an antigen binding protein is covalently modified to include one or more polymers, including, but not limited to, monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers. See, e.g., U.S. Pat. Nos. 4,640,835, 4,496,689, 4,301,144, 4,670,417, 4,791,192 and 4,179,337.

The term “compete” when used in the context of antigen binding proteins (e.g., antibodies or fragments thereof) that compete for the same epitope means competition between antigen binding proteins as determined by an assay in which the antigen binding protein (e.g., an antibody or fragment thereof) being tested (e.g., a test antibody) prevents or inhibits (partially or completely) specific binding of a reference antigen binding protein (e.g., a reference antibody) to a common antigen (e.g., gpNMB or a fragment thereof). Numerous types of competitive binding assays can be used to determine if one antigen binding protein competes with another, including various label-free biosensor approaches such as surface plasmon resonance (SPR) analysis (see, e.g., Abdiche, et al., 2009, Anal. Biochem. 386:172-180; Abdiche, et al., 2012, J. Immunol Methods 382:101-116; and Abdiche, et al., 2014 PLoS One 9:e92451. Other assays that can be used include: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using I-125 label (see, e.g., Morel et al., 1988, Mol. Immunol. 25:7-15); solid phase direct biotin-avidin EIA (see, e.g., Cheung, et al., 1990, Virology 176:546-552); direct labeled RIA (Moldenhauer et al., 1990, Scand. J. Immunol. 32:77-82). Typically, the test antigen binding protein is present in excess (e.g., at least 2×, 5×, 10×, 20× or 100×). Usually, when a competing antigen binding protein is present in excess, it will inhibit specific binding of a reference antigen binding protein to a common antigen by at least 40%, 50%, 60%, 70%, 80%, 90%, 95% or 100%. In instances in in which each antigen binding protein (e.g., an antibody or fragment thereof) detectably inhibits the binding of the other antigen binding protein with its cognate epitope, whether to the same, greater, or lesser extent, the antigen binding proteins are said to “cross-compete” with each other for binding of their respective epitope(s) or to “cross-block” one another. Typically, such cross-competition studies are done using the conditions and methods described above for competition studies and the extent of blocking is at least 30%, at least 40%, or at least 50% each way.

An “affinity matured” antibody refers to an antibody with one or more alterations in one or more hypervariable regions (HVRs) compared to a parent antibody which does not possess such alterations, such alterations resulting in an improvement in the affinity of the antibody for antigen. In some examples, an affinity matured antibody refers to an antibody with one or more alterations in one or more complementarity determining regions (CDRs) compared to a parent antibody which does not possess such alterations, such alterations resulting in an improvement in the affinity of the antibody for antigen.

As used herein, the term “specifically binds”, “binding” or simply “binds” or other related terms in the context of the binding of an antigen binding protein to its target antigen means that the antigen binding protein exhibits essentially background binding to non-target molecules. An antigen binding protein that specifically binds a target antigen (e.g., gpNMB) may, however, cross-react with the corresponding protein (such as gpNMB) from different species.

The term “KD” (M), as used herein, refers to the dissociation equilibrium constant of a particular antigen binding protein-antigen interaction (e.g., antibody-antigen interaction). Affinity, as used herein, and K_{D} are inversely related, such that higher affinity is intended to refer to lower K_{D}, and lower affinity is intended to refer to higher K_{D}.

An “antibody-drug-conjugate” or simply “ADC” refers to an antibody conjugated to a cytotoxic agent or cytostatic agent. An antibody-drug-conjugate typically binds to the target antigen (e.g., gpNMB) on a cell surface followed by internalization of the antibody-drug-conjugate into the cell where the drug is released.

A “cytotoxic effect” refers to the depletion, elimination and/or killing of a target cell.

A “cytotoxic agent” refers to an agent that has a cytotoxic effect on a cell.

A “cytostatic effect” refers to the inhibition of cell proliferation.

A “cytostatic agent” refers to an agent that has a cytostatic effect on a cell, thereby inhibiting the growth of and/or expansion of a specific subset of cells. Cytostatic agents can be conjugated to an antibody or administered in combination with an antibody.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

A “functional Fc region” possesses an “effector function” of a native sequence Fc region. Exemplary “effector functions” include Fc receptor binding; C1q binding; complement dependent cytotoxicity (CDC); antibody-dependent cell-mediated cytotoxicity (ADCC); antibody-dependent cellular phagocytosis (ADCP); down regulation of cell surface receptors (e.g. B cell receptor; BCR), etc. Such effector functions generally require the Fc region to be combined with a binding domain (e.g., an antibody variable domain) and can be assessed using various assays.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification.

“Fc receptor” or “FcR” describes a receptor that binds to the Fc region of an antibody. In some embodiments, an FcγR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

“Effector functions” refer to biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); antibody-dependent cellular phagocytosis (ADCP); down regulation of cell surface receptors (e.g. B cell receptor); and B cell activation. Such functions can be affected by, for example, binding of an Fc effector domain(s) to an Fc receptor on an immune cell with phagocytic or lytic activity or by binding of an Fc effector domain(s) to components of the complement system. Typically, the effect(s) mediated by the Fc-binding cells or complement components result in inhibition and/or depletion of the CD33 targeted cell. Fc regions of antibodies can recruit Fc receptor (FcR)-expressing cells and juxtapose them with antibody-coated target cells. Cells expressing surface FcR for IgGs including FcγRIII (CD16), FcγRII (CD32) and FcγRIII (CD64) can act as effector cells for the destruction of IgG-coated cells. Such effector cells include monocytes, macrophages, natural killer (NK) cells, neutrophils and eosinophils. Engagement of FcγR by IgG activates antibody-dependent cellular cytotoxicity (ADCC) or antibody-dependent cellular phagocytosis (ADCP). ADCC is mediated by CD16⁺ effector cells through the secretion of membrane pore-forming proteins and proteases, while phagocytosis is mediated by CD32⁺ and CD64⁺ effector cells (see, e.g., Fundamental Immunology, 4^{th} ed., Paul ed., Lippincott-Raven, N.Y., 1997, Chapters 3, 17 and 30; Uchida et al., 2004, J. Exp. Med. 199:1659-69; Akewanlop et al., 2001, Cancer Res. 61:4061-65; Watanabe et al., 1999, Breast Cancer Res. Treat. 53:199-207.

“Human effector cells” are leukocytes which express one or more FcRs and perform effector functions. In certain embodiments, the cells express at least FcγRIII and perform ADCC effector function(s). Examples of human leukocytes which mediate ADCC include peripheral blood mononuclear cells (PBMC), natural killer (NK) cells, monocytes, cytotoxic T cells, and neutrophils. The effector cells may be isolated from a native source, e.g., from blood.

“Antibody-dependent cell-mediated cytotoxicity” or “ADCC” refers to a mechanism of cytotoxicity in which the Fc region of antibodies bound to antigen on the cell surface of target cells interact with Fc receptors (FcRs) present on certain cytotoxic effector cells (e.g. NK cells, neutrophils, and macrophages). This interaction enables these cytotoxic effector cells to subsequently kill the target cell with cytotoxins. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or U.S. Pat. No. 5,821,337 or U.S. Pat. No. 6,737,056 (Presta), can be performed. Useful effector cells for such assays include PBMC and NK cells. ADCC activity of the molecule of interest can also be assessed in vivo, e.g., in an animal model such as that disclosed in Clynes et al. Proc. Natl. Acad. Sci. (USA) 95:652-656 (1998). Additional polypeptide variants with altered Fc region amino acid sequences (polypeptides with a variant Fc region) and increased or decreased ADCC activity are described, e.g., in U.S. Pat. Nos. 7,923,538, and 7,994,290.

“Complement dependent cytotoxicity” or “CDC” refers to the lysis of a target cell in the presence of complement. Activation of the classical complement pathway is initiated by the binding of the first component of the complement system (C1q) to the Fc region of antibodies (of the appropriate subclass), which are bound to their cognate antigen on a target cell. This binding activates a series of enzymatic reactions culminating in the formation of holes in the target cell membrane and subsequent cell death. Activation of complement may also result in deposition of complement components on the target cell surface that facilitate ADCC by binding complement receptors (e.g., CR3) on leukocytes. To assess complement activation, a CDC assay, e.g., as described in Gazzano-Santoro et al., J. Immunol. Methods 202:163 (1996), can be performed. Polypeptide variants with altered Fc region amino acid sequences (polypeptides such as an antibody with a variant Fc region) and increased or decreased C1q binding capability are described, e.g., in U.S. Pat. No. 6,194,551 B1, U.S. Pat. Nos. 7,923,538, 7,994,290 and WO 1999/51642. See also, e.g., Idusogie et al., J. Immunol. 164: 4178-4184 (2000).

The term “antibody-dependent cellular phagocytosis”, or simply “ADCP”, refers to the process by which antibody-coated cells are internalized, either in whole or in part, by phagocytic immune cells (e.g., macrophages, neutrophils and dendritic cells) that bind to an Fc region of Ig.

A polypeptide variant with “altered” FcR binding affinity or ADCC activity (e.g., an antibody) is one which has either enhanced or diminished FcR binding activity and/or ADCC activity compared to a parent polypeptide or to a polypeptide comprising a native sequence Fc region. The polypeptide variant which “displays increased binding” to an FcR binds at least one FcR with better affinity than the parent polypeptide. The polypeptide variant which “displays decreased binding” to an FcR, binds at least one FcR with lower affinity than a parent polypeptide. In some embodiments, such variants which display decreased binding to an FcR may possess little or no appreciable binding to an FcR, e.g., 0-20% binding to the FcR compared to a native sequence IgG Fc region.

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

“Affinity” refers to the strength of the sum total of noncovalent interactions between a single binding site of a molecule (e.g., an antibody) and its binding partner (e.g., an antigen). The affinity of a molecule X for its partner Y can generally be represented by the dissociation constant (K_{d}). Affinity can be measured by common methods known in the art, including those described herein.

An “autoimmune disease” as used herein refers to a disease or disorder arising from and directed against an individual's own tissues or proteins.

“Patient” as used herein refers to a subject to whom is administered a Ligand-Drug Conjugate compound of the present invention. Patient includes, but are not limited to, a human, rat, mouse, guinea pig, non-human primate, pig, goat, cow, horse, dog, cat, bird and fowl. Typically, the patient is a rat, mouse, dog, human or non-human primate, more typically a human.

The terms “treat” or “treatment,” unless otherwise indicated by context, refer to therapeutic treatment and prophylactic wherein the object is to inhibit or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder.

In the context of cancer, the term “treating” includes any or all of: killing tumor cells; inhibiting growth of tumor cells, cancer cells, or of a tumor; inhibiting replication of tumor cells or cancer cells, lessening of overall tumor burden or decreasing the number of cancerous cells, and ameliorating one or more symptoms associated with the disease.

In the context of an autoimmune disease, the term “treating” includes any or all of: inhibiting replication of cells associated with an autoimmune disease state including, but not limited to, cells that produce an autoimmune antibody, lessening the autoimmune-antibody burden and ameliorating one or more symptoms of an autoimmune disease.

“Compound” as the term is used herein, refers to and encompasses the chemical compound itself, either named or represented by structure, and salt form(s) thereof, whether explicitly stated or not, unless context makes clear that such salt forms are to be excluded. The term “compound” further encompasses solvate forms of the compound, in which solvent is noncovalently associated with the compound or is reversibly associated covalently with the compound, as when a carbonyl group of the compound is hydrated to form a gem-diol. Solvate forms include those of the compound itself and its salt form(s) and are inclusive of hemisolvates, monosolvates, disolvates, including hydrates; when a compound is associated with two or more solvent molecules, the two or more solvent molecules are the same or different.

In some instances, a compound of the invention will include an explicit reference to one or more of the above forms, e.g., salts and solvates, which does not imply any solid state form of the compound; however, this reference is for emphasis only, and is not to be construed as excluding any other of the forms as identified above. Furthermore, when explicit reference to a salt and/or solvate form of a compound or a Ligand Drug Conjugate composition is not made, that omission is not to be construed as excluding the salt and/or solvate form(s) of the compound or Conjugate unless context make clear that such salt and/or solvate forms are to be excluded.

The phrase “salt thereof” as the phrase is used herein, refers to a salt form of a compound (e.g., a Drug, a Drug-Linker compound or a Ligand-Drug Conjugate compound). A salt form of a compound is of one or more internal salt forms and/or involves the inclusion of another molecule such as an acetate ion, a succinate ion or other counterion. The counterion in a salt form of a compound is typically an organic or inorganic moiety that stabilizes the charge on the parent compound. A salt form of a compound has one or more than one charged atom in its structure. In instances where multiple charged atoms are part of the salt form, multiple counter ions and/or multiple charged counter ions are present. Hence, a salt form of a compound typically has one or more charged atoms corresponding to those of the non-salt form of the compound and one or more counterions. In some aspects, the non-salt form of a compound contains at least one amino group or other basic moeity, and accordingly in the presence of an acid, an acid addition salt with the basic moiety is obtained. In other aspects, the non-salt form of a compound contains at least one carboxylic acid group or other acidic moiety, and accordingly in the presence of a base, a carboxylate or other anionic moiety is obtained. Exemplary salts include, but are not limited to, sulfate, trifluoroacetate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

A pharmaceutically acceptable salt is a salt form of a compound that is suitable for administration to a subject as described herein and in some aspects includes countercations or counteranions as described by P. H. Stahl and C. G. Wermuth, editors, Handbook of Pharmaceutical Salts: Properties, Selection and Use, Weinheim/Zürich: Wiley-VCH/VHCA, 2002.

A “Linker Unit” as used herein is a bifunctional moiety that connects or is capable of connecting a Drug Unit to a Ligand Unit in a Ligand-Drug Conjugate compound. The Linker Units of the present invention comprise two or more components selected from the group consisting of a Stretcher Unit which in some embodiments will have a Basic Unit; a Connector Unit; a Parallel Connector Unit; a Releasable Linker; and a Spacer Unit.

“PEG”, “PEG Unit” or “polyethylene glycol” as used herein is an organic moiety comprising repeating ethylene-oxy subunits and is polydisperse, monodisperse, or discrete (i.e., having discrete number of ethylene-oxy subunits). Polydisperse PEGs are a heterogeneous mixture of sizes and molecular weights whereas monodisperse PEGs are typically purified from heterogeneous mixtures and are therefore provide a single chain length and molecular weight. Preferred PEG Units are discrete PEGs, compounds that are synthesized in stepwise fashion and not via a polymerization process. Discrete PEGs provide a single molecule with defined and specified chain length.

The PEG Unit provided herein comprises one or multiple polyethylene glycol chains, each comprising one or more ethyleneoxy subunits, covalently attached to each other. The polyethylene glycol chains are linked together in any pattern (e.g., in a linear, branched, or star shaped configuration). Typically, at least one of the polyethylene glycol chains prior to incorporation into a Ligand-Drug Conjugate compound is derivitized at one end with an alkyl moiety substituted with an electrophilic group for covalent attachment to the carbamate nitrogen of a methylene carbamate unit (i.e., represents an instance of R). Typically, the terminal ethyleneoxy subunit in each polyethylene glycol chains not involved in covalent attachment to the remainder of the Linker Unit is modified with a PEG Capping Unit, typically H or an optionally substituted alkyl such as —CH₃, —CH₂CH₃ or —CH₂CH₂CO₂H. A preferred PEG Unit has a single polyethylene glycol chain with 4 to 24 —CH₂CH₂O— subunits covalently attached in series and terminated at one end with a PEG Capping Unit.

“Halogen” as the term is used herein by itself or in combination with another term, unless otherwise stated or implied by context, refers to fluorine, chlorine, bromine or iodine and is typically —F or —Cl.

Unless otherwise indicated, the term “alkyl” by itself or as part of another term refers to a straight chain or branched, saturated hydrocarbon having the indicated number of carbon atoms (e.g., “—C₁-C₄ alkyl,” “—C₁-C₈ alkyl,” or “—C₁-C₁₀” alkyl refer to an alkyl group having from 1 to 4, 1 to 8, or 1 to 10 carbon atoms, respectively). When the number of carbon atoms is not indicated, the alkyl group has from 1 to 8 carbon atoms. Representative straight chain “—C₁-C₈ alkyl” groups include, but are not limited to, -methyl, -ethyl, -n-propyl, -n-butyl, -n-pentyl, -n-hexyl, -n-heptyl and -n-octyl; while branched —C₃-C₈ alkyls include, but are not limited to, -isopropyl, -sec-butyl, -isobutyl, -tert-butyl, -isopentyl, and -2-methylbutyl.

Unless otherwise indicated, “alkylene,” by itself of as part of another term, refers to a saturated, branched or straight chain or cyclic hydrocarbon radical of the stated number of carbon atoms, typically 1-4, 1-8, or 1-10 carbon atoms, and having two monovalent radical centers derived by the removal of two hydrogen atoms from the same or two different carbon atoms of a parent alkane. Typical alkylene radicals include, but are not limited to: methylene (—CH₂—), 1,2-ethylene (—CH₂CH₂—), 1,3-propylene (—CH₂CH₂CH₂—), 1,4-butylene (—CH₂CH₂CH₂CH₂—), and the like. In preferred aspects, an alkylene is a branched or straight chain hydrocarbon (i.e., it is not a cyclic hydrocarbon).

“Alkenyl” as the term is used herein, by itself or as part of another term, unless otherwise stated or implied by context, refers to an organic moiety, substituent or group that comprises one or more double bond functional groups (e.g., a —CH═CH— moiety) or 1, 2, 3, 4, 5 or 6 or more, typically 1, 2 or 3 of such functional groups, more typically one such functional group, and in some aspects may contain non-aromatic linked normal, secondary, tertiary or cyclic carbon atoms, i.e., linear, branched, cyclic or any combination thereof as part of the base moiety unless the alkenyl substituent, moiety or group is a vinyl moiety (e.g., a —CH═CH₂ moiety). An alkenyl moiety, group or substituent having multiple double bonds may have the double bonds arranged contiguously (i.e., a 1,3-butadienyl moiety) or non-contiguously with one or more intervening saturated carbon atoms or a combination thereof, provided that a cyclic, contiguous arrangement of double bonds do not form a cyclic conjugated system of 4n+2 electrons (i.e., is not aromatic).

An alkenyl moiety, group or substituent contains at least one sp² carbon atom in which that carbon atom is divalent and is doubly bonded to another organic moiety or Markush structure to which it is associated, or contains at least two sp² carbon atoms in conjugation to each other in which one of the sp² carbon atoms is monovalent and is singly bonded to another organic moiety or Markush structure to which it is associated. Typically, when alkenyl is used as a Markush group (i.e., is a substituent) the alkenyl is singly bonded to a Markush formula or another organic moiety with which it is associated through a sp² carbon of an alkene functional group of the alkenyl moiety. In some aspects, when an alkenyl moiety is specified, species encompasses those corresponding to any of the optionally substituted alkyl or carbocyclyl, groups moieties or substituents described herein that has one or more endo double bonds in which a sp² carbon atom thereof is monovalent and monovalent moieties derived from removal of a hydrogen atom from a sp² carbon of a parent alkene compound. Such monovalent moieties are exemplified without limitation by vinyl (—CH═CH₂), allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, and cyclohexenyl. In some aspects, the term alkenyl encompasses those and/or other linear, cyclic and branched chained, all carbon-containing moieties containing at least one double bond functional group in which one of the sp² carbon atoms is monovalent.

The number of carbon atoms in an alkenyl moiety is defined by the number of sp² carbon atoms of the alkene functional group(s) that defines it as an alkenyl substituent and the total number of contiguous non-aromatic carbon atoms appended to each of these sp² carbons not including any carbon atom of the other moiety or Markush structure for which the alkenyl moiety is a variable group and carbon atoms from any optional substituent to the alkenyl moiety. That number ranges from 1 to 50 or 1 to 30, typically 1 to 20 or 1 to 12, more typically, 1 to 8, 1 to 6 or 1 to 4 carbon atoms when the double bond functional group is doubly bonded to a Markush structure (e.g. ═CH₂), or ranges from 2 to 50, typically 2 to 30, 2 to 20 or 2 to 12, more typically 2 to 8, 2 to 6 or 2 to 4 carbon atoms, when the double bond functional group is singly bonded to the Markush structure (e.g., —CH═CH₂). For example, C₂-C₈ alkenyl or C2-C8 alkenyl means an alkenyl moiety containing 2, 3, 4, 5, 6, 7 or 8 carbon atoms in which at least two are sp² carbon atoms in conjugation with each other with one of these carbon atoms being monovalent, and C₂-C₆ alkenyl or C2-C6 alkenyl means an alkenyl moiety containing 2, 3, 4, 5 or 6 carbon atoms in which at least two are sp² carbons that are in conjugation with each other with one of these carbon atoms being monovalent. In some aspects, an alkenyl substituent or group is a C₂-C₆ or C₂-C₄ alkenyl moiety having only two sp² carbons that are in conjugation with each other with one of these carbon atoms being monovalent. Typically, an alkenyl substituent is a C₂-C₆ or C₂-C₄ alkenyl moiety having only two sp² carbons that are in conjugation with each other. When the number of carbon atoms is not indicated, an alkenyl moiety has from 2 to 8 carbon atoms.

“Alkenylene” as the term is used herein, by itself of as part of another term, unless otherwise stated or implied by context, refers to an organic moiety, substituent or group that comprises one or more double bond moieties, as previously described for alkenyl, of the stated number of carbon atoms and has two radical centers derived by the removal of two hydrogen atoms from the same or two different sp² carbon atoms of an alkene functional group or removal of two hydrogen atoms from two separate alkene functional groups in a parent alkene. In some aspects, an alkenylene moiety is that of an alkenyl radical as described herein in which a hydrogen atom has been removed from the same or different sp² carbon atom of a double bond functional group of the alkenyl radical, or from a sp² carbon from a different double bonded moiety to provide a diradical. Typically, alkenylene moieties encompass diradicals containing the structure of —C═C— or —C≡C—X¹—C≡C— wherein X¹ is absent or is an alkylene as defined herein, which is typically a C₁-C₆ alkylene. The number of carbon atoms in an alkenylene moiety is defined by the number of sp² carbon atoms of its alkene functional group(s) that defines it as an alkenylene moiety and the total number of contiguous non-aromatic carbon atoms appended to each of its sp² carbons not including any carbon atoms of the other moiety or Markush structure in which the alkenyl moiety is a present as a variable group. That number, unless otherwise specified, ranges from 2 to 50 or 2 to 30, typically from 2 to 20 or 2 to 12, more typically from 2 to 8, 2 to 6 or 2 to 4 carbon atoms. For example, C₂-C₈ alkenylene or C2-C8 alkenylene means an alkenylene moiety containing 2, 3, 4, 5, 6, 7 or 8 carbon atoms, in which at least two are sp² carbons in which one is divalent or both are monovalent, that are in conjugation with each other and C₂-C₆ alkenylene or C2-C6 alkenylene means an alkenyl moiety containing 2, 3, 4, 5 or 6 carbon atoms in which at least two are sp² carbons, in which at least two are sp² carbons in which one is divalent or both are monovalent, that are in conjugation with each other. In some aspects, an alkenylene moiety is a C₂-C₆ or C₂-C₄ alkenylene having two sp² carbons that are in conjugation with each other in which both sp² carbon atoms are monovalent. When the number of carbon atoms is not indicated, an alkenylene moiety has from 2 to 8 carbon atoms.

“Alkynyl” as the term is used herein, by itself or as part of another term, unless otherwise stated or implied by context, refers to an organic moiety, substituent or group that comprises one or more triple bond functional groups (e.g., a —C≡C— moiety) or 1, 2, 3, 4, 5, or 6 or more, typically 1, 2, or 3 of such functional groups, more typically one such functional group. An alkynyl moiety, group or substituent having multiple triple bonds may have the triple bonds arranged contiguously or non-contiguously with one or more intervening saturated or unsaturated carbon atoms or a combination thereof, provided that a cyclic, contiguous arrangement of triple bonds do not form a cyclic conjugated system of 4n+2 electrons (i.e., is not aromatic).

An alkynyl moiety, group or substituent contains at least two sp carbon atom in which the carbon atoms are conjugation to each other and in which one of the sp carbon atoms is singly bonded, to another organic moiety or Markush structure to which it is associated. When alkynyl is used as a Markush group (i.e., is a substituent) the alkynyl is singly bonded to a Markush formula or another organic moiety with which it is associated through a triple-bonded carbon (i.e., a sp carbon) of a terminal alkyne functional group. In some aspects when an alkynyl moiety, group or substituent is specified, species encompasses are any of the alkyl or carbocyclyl, groups moieties or substituents described herein that has one or more endo triple bonds and monovalent moieties derived from removal of a hydrogen atom from a sp carbon of a parent alkyne compound. Such monovalent moieties are exemplified without limitation by —C≡CH, and —C≡C—CH₃, and —C≡C-Ph.

The number of carbon atoms in an alkynyl substituent is defined by the number of sp carbon atoms of the alkene functional group that defines it as an alkynyl substituent and the total number of contiguous non-aromatic carbon atoms appended to each of these sp carbons not including any carbon atom of the other moiety or Markush structure for which the alkenyl moiety is a variable group. That number can vary ranging from 2 to 50, typically 2 to 30, 2 to 20, or 2 to 12, more typically 2 to 8, 2 to 6, or 2 to 4 carbon atoms, when the triple bond functional group is singly bonded to the Markush structure (e.g., —CH≡CH). For example, C₂-C₈ alkynyl or C2-C8 alkynyl means an alkynyl moiety containing 2, 3, 4, 5, 6, 7, or 8 carbon atoms in which at least two are sp carbon atoms in conjugation with each other with one of these carbon atoms being monovalent, and C₂-C₆ alkynyl or C2-C6 alkynyl means an alkynyl moiety containing 2, 3, 4, 5, or 6 carbon atoms in which at least two are sp carbons that are in conjugation with each other with one of these carbon atoms being monovalent. In some aspects, an alkynyl substituent or group is a C₂-C₆ or C₂-C₄ alkynyl moiety having two sp carbons that are in conjugation with each other with one of these carbon atoms being monovalent. When the number of carbon atoms is not indicated, an alkynyl moiety, group or substituent has from 2 to 8 carbon atoms.

The term “Prodrug” as used herein refers to a less biologically active or inactive compound which is transformed within the body into a more biologically active compound via a chemical or biological process (i.e., a chemical reaction or an enzymatic biotransformation). Typically, a biologically active compound is rendered less biologically active (i.e., is converted to a prodrug) by chemically modifying the compound with a prodrug moiety. In some aspects, the prodrug is a Type II prodrug, which are bioactivated outside cells, e.g., in digestive fluids, or in the body's circulation system, e.g., in blood. Exemplary prodrugs are esters and β-D-glucopyranosides.

Unless otherwise indicated, “aryl,” by itself or as part of another term, means a monovalent carbocyclic aromatic hydrocarbon radical of the stated number of carbon atoms, typically 6-20 carbon atoms, derived by the removal of one hydrogen atom from a single carbon atom of a parent aromatic ring system. Some aryl groups are represented in the exemplary structures as “Ar”. Typical aryl groups include, but are not limited to, radicals derived from benzene, naphthalene, anthracene, biphenyl, and the like. An exemplary aryl group is a phenyl group.

Unless otherwise indicated, an “arylene,” by itself or as part of another term, is an aryl group as defined above which has two covalent bonds (i.e., it is divalent) and is in the ortho, meta, or para orientations as shown in the following structures, with phenyl as the exemplary group:

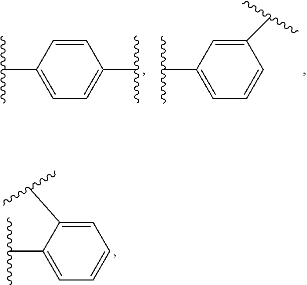

Unless otherwise indicated, a “C₃-C₈ heterocycle,” by itself or as part of another term, refers to a monovalentaromatic or non-aromatic monocyclic or bicyclic ring system having from 3 to 8 carbon atoms (also referred to as ring members) and one to four heteroatom ring members independently selected from N, O, P or S, and derived by removal of one hydrogen atom from a ring atom of a parent ring system. In some aspects, one or more N, C or S atoms in the heterocycle is/are oxidized. In some aspects, the ring that includes the heteroatom is aromatic or nonaromatic. Heterocycles in which all the ring atoms are involved in aromaticity are referred to as heteroaryls and otherwise are referred to heterocarbocycles.

Unless otherwise noted, the heterocycle is attached to its pendant group at any heteroatom or carbon atom that results in a stable structure. In some aspects, a heteroaryl is bonded through an aromatic carbon of its aromatic ring system, referred to as a C-linked heteroaryl. In other aspects, a heteroaryl is bonded through a non-double-bonded N atom (i.e., not ═N—) in its aromatic ring system, which is referred to as an N-linked heteroaryl. Thus, nitrogen-containing heterocycles are C-linked or N-linked and include pyrrole moieties, such as pyrrol-1-yl (N-linked) and pyrrol-3-yl (C-linked), and imidazole moieties such as imidazol-1-yl and imidazol-3-yl (both N-linked), and imidazol-2-yl, imidazol-4-yl and imidazol-5-yl moieties (all of which are C-linked).

Unless otherwise indicated, a “C₃-C₈ heteroaryl,” is an aromatic C₃-C₈ heterocycle in which the subscript denotes the total number of carbons of the cyclic ring system of the heterocycle or the total number of aromatic carbons of the aromatic ring system of the heteroaryl and does not implicate the size of the ring system or the presence or absence of ring fusion. Representative examples of a C₃-C₈ heterocycle include, but are not limited to, pyrrolidinyl, azetidinyl, piperidinyl, morpholinyl, tetrahydrofuranyl, tetrahydropyranyl, benzofuranyl, benzothiophene, indolyl, benzopyrazolyl, pyrrolyl, thiophenyl (thiophene), furanyl, thiazolyl, imidazolyl, pyrazolyl, pyrimidinyl, pyridinyl, pyrazinyl, pyridazinyl, isothiazolyl, and isoxazolyl.

When explicitly given, the size of the ring system of a heterocycle or heteroaryl is indicated by the total number of atoms in the ring. For example, designation as a 5- or 6-membered heteroaryl indicates the total number of aromatic atoms (i.e., 5 or 6) in the heteroaromatic ring system of the heteroaryl but does not imply the number of aromatic heteroatoms or the number of aromatic carbon atoms in that ring system. Fused heteroaryls are explicitly stated or implied by context as such and are typically indicated by the number of aromatic atoms in each aromatic ring that are fused together to make up the fused heteroaromatic ring system. For example, a 5,6-membered heteroaryl is an aromatic 5-membered ring fused to an aromatic 6-membered ring in which one or both rings have aromatic heteroatom(s) or where a heteroatom is shared between the two rings.

A heterocycle fused to an aryl or heteroaryl such that the heterocycle remains non-aromatic and is part of a larger structure through attachment with the non-aromatic portion of the fused ring system is an example of a heterocycle in which the heterocycle is substituted by ring fusion with the aryl or heteroaryl. Likewise, an aryl or heteroaryl fused to heterocycle or carbocycle that is part of a larger structure through attachment with the aromatic portion of the fused ring system is an example of an aryl or heterocycle in which the aryl or heterocycle is substituted by ring fusion with the heterocycle or carbocycle.

Unless otherwise indicated, “C₃-C₈ heterocyclo,” by itself or as part of another term, refers to a C₃-C₈ heterocyclic defined above wherein one of the hydrogen atoms of the heterocycle is replaced with a bond (i.e., it is divalent). Unless otherwise indicated, a “C₃-C₈ heteroarylene,” by itself or as part of another term, refers to a C₃-C₈ heteroaryl group defined above wherein one of the heteroaryl group's hydrogen atoms is replaced with a bond (i.e., it is divalent). When explicitly given, the size of the ring system of a heteroarylene is indicated by the total number of atoms in the ring. For example, designation as a 5- or 6-membered heteroarylene indicates the total number of atoms (i.e., 5 or 6) in the heterocyclic ring system of the heterocycle, but does not imply the number of heteroatoms or the number of carbon atoms in that ring system.

Unless otherwise indicated, a “C₃-C₈ carbocycle,” by itself or as part of another term, is a 3-, 4-, 5-, 6-, 7- or 8-membered monovalent, saturated or unsaturated non-aromatic monocyclic or bicyclic carbocyclic ring derived by the removal of one hydrogen atom from a ring atom of a parent ring system. Representative —C₃-C₈ carbocycles include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl.

Unless otherwise indicated, a “C₃-C₈ carbocyclo,” by itself or as part of another term, refers to a C₃-C₈ carbocycle group defined above wherein another one of the carbocycle group's hydrogen atoms is replaced with a bond (i.e., it is divalent).

Unless otherwise indicated, the term “heteroalkyl,” by itself or in combination with another term, means, unless otherwise stated, a stable straight or branched chain hydrocarbon, or combinations thereof, fully saturated or containing from 1 to 3 degrees of unsaturation, consisting of the stated number of carbon atoms and from one to ten, preferably one to three, heteroatoms selected from the group consisting of O, N, Si and S, and wherein the nitrogen and sulfur atoms are optionally oxidized and the nitrogen heteroatom is optionally quaternized. The heteroatom(s) O, N and S are placed at any interior position of the heteroalkyl group or at the position at which the alkyl group is attached to the remainder of the molecule. The heteroatom Si is placed at any position of the heteroalkyl group, including the position at which the alkyl group is attached to the remainder of the molecule. When explicitly given, the number of atoms in a heteroalkyl or heteroarylene is indicated by the total number of atoms in the group. For example, designation as a C₁-C₂ heteroalkyl indicates the total number of atoms (i.e., 1 or 2) in the heteroalkyl group, but does not imply the number of heteroatoms or the number of carbon atoms in that group.

Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —NH—CH₂—CH₂—NH—C(O)—CH₂—CH₃, —CH₂—CH₂—S(O)₂—CH₃, —CH═CH—O—CH₃, —Si(CH₃)₃, —CH₂—CH═N—O—CH₃, and —CH═CH—N(CH₃)—CH₃. In some aspects, two heteroatoms are consecutive, such as, for example, —CH₂—NH—OCH₃ and —CH₂—O—Si(CH₃)₃. Typically, a C₁ to C₄ heteroalkyl or heteroalkylene has 1 to 4 carbon atoms and 1 or 2 heteroatoms and a C₁ to C₃ heteroalkyl or heteroalkylene has 1 to 3 carbon atoms and 1 or 2 heteroatoms. In some aspects, a heteroalkyl or heteroalkylene is saturated.

Unless otherwise indicated, the term “heteroalkylene” by itself or in combination with another term means a divalent group derived from heteroalkyl (as discussed above), as exemplified by —CH₂—CH₂—S—CH₂—CH₂— and —CH₂—S—CH₂—CH₂—NH—CH₂—. For heteroalkylene groups, heteroatoms can also occupy either or both of the chain termini. Still further, for alkylene and heteroalkylene linking groups, no orientation of the linking group is implied.

Unless otherwise indicated, “aminoalkyl” by itself or in combination with another term means a heteroalkyl wherein an alkyl moiety as defined herein is substituted with an amino, alkylamino, dialkylamino or cycloalkylamino group. Exemplary non-limiting aminoalkyls are —CH₂NH₂, —CH₂CH₂NH₂, —CH₂CH₂NHCH₃ and —CH₂CH₂N(CH₃)₂ and further includes branched species such as —CH(CH₃)NH₂ and —C(CH₃)CH₂NH₂ in the (R)- or (S)-configuration. Alternatively, an aminoalkyl is an alkyl moiety, group, or substituent as defined herein wherein a sp³ carbon other than the radical carbon atom of the alkyl moiety has been replaced with an amino or alkylamino moiety wherein its sp³ nitrogen atom replaces the sp³ carbon of the alkyl moiety provided that at least one sp³ carbon atom of the alkyl moiety remains. When referring to an aminoalkyl moiety as a substituent to a larger structure or another moiety the aminoalkyl is covalently attached to the structure or moiety through the carbon radical of the alkyl moiety of the aminoalkyl.

“Hydroxyalkyl” as the term is used herein by itself or in combination with another term, unless otherwise stated or implied by context, refers to an alkyl moiety, group, or substituent having a hydroxyl radical in place of one or more hydrogen atoms of the alkyl moiety, group, or substituent. In some aspects, one or two hydrogen atoms are each replaced with a hydroxyl substituent in a hydroxyalkyl group. A hydroxyalkyl is typically denoted by the number of contiguous carbon atoms of its alkyl or alkylene moiety. Thus, a C₁ hydroxyalkyl is exemplified without limitation by —CH₂OH, and a C₂ hydroxyalkyl is exemplified without limitation by —CH₂CH₂OH or —CH₂(OH)CH₃.

“Haloalkyl” as the term is used herein by itself or in combination with another term, unless otherwise stated or implied by context, refers to an alkyl moiety, group, or substituent having a halogen in place of one or more hydrogen atoms of the alkyl moiety, group, or substituent. In some aspects, one or two hydrogen atoms are each replaced with a halogen in a haloalkyl group. A haloalkyl is typically denoted by the number of contiguous carbon atoms of its alkyl or alkylene moiety. Thus, a C₁ haloalkyl is exemplified without limitation by —CH₂F, —CH₂Cl, —CH₂Br, or —CH₂I, and a C₂ haloalkyl is exemplified without limitation by —CH₂CH₂F, —CH₂CH₂Cl, —CH₂CH₂Br, —CH₂CH₂I, —CH₂(F)CH₃, —CH₂(Cl)CH₃, —CH₂(Br)CH₃, or —CH₂(I)CH₃. In some embodiments, the term “haloalkyl” refers to an alkyl moiety, group, or substituent having halogens in place of two or more hydrogen atoms. For example, a C₁ haloalkyl is also exemplified without limitation by —CHF₂, —CHCl₂, —CHBr₂, or —CHI₂, and a C₂ haloalkyl is exemplified without limitation by —CH₂CHF₂, —CH₂CHCl₂, —CH₂CHBr₂, —CH₂CHI₂, —CH(F)₂CH₃, —CH(Cl)₂CH₃, —CH(Br)₂CH₃, or —CH(I)₂CH₃. In some aspects, the term “haloalkyl” refers to an alkyl moiety, group, or substituent having halogens in place of all hydrogen atoms. Thus, in some aspects, the term “haloalkyl” encompasses fully halogenated alkyl moieties, groups, or substituents. For example, a C₁ haloalkyl is also exemplified without limitation by —CF₃, —CCl₃, —CBr₃, or —CI₃.

Unless otherwise indicated “alkylamino” and “cycloalkylamino” by itself or in combination with another term means an alkyl or cycloalkyl radical, as described herein, wherein the radical carbon atom of the alkyl or cycloalkyl radical has been replaced with a nitrogen radical, provided that at least one sp³ carbon atom of the alkyl or cycloalkyl radical remains. In those instances where the alkylamino is substituted at its nitrogen with another alkyl moiety the resulting substituted radical is sometimes referred to as a dialkylamino moiety, group or substituent wherein the alkyl moieties substituting nitrogen are independently selected.

Exemplary and non-limiting amino, alkylamino and dialkylamino substituents, include those having the structure of —N(R′)₂, wherein R′ in these examples are independently hydrogen or C₁₋₆ alkyl, typically hydrogen or methyl, whereas in cycloalkyl amines, which are included in heterocycloalkyls, both R′ together with the nitrogen to which they are attached define a heterocyclic ring. When both R′ are hydrogen or alkyl, the moiety is sometimes described as a primary amino group and a tertiary amine group, respectively. When one R′ is hydrogen and the other is alkyl, then the moiety is sometimes described as a secondary amino group. Primary and secondary alkylamino moieties are typically more reactive as nucleophiles towards carbonyl-containing electrophilic centers whereas tertiary amines are typically more basic.

The term “substituted” means that the specified group or moiety bears one or more substituents. Typical substituents include, but are not limited to a —X, —R″, —OH, —OR″, —SR″, —N(R″)₂, —N(R″)₃, ═NR″, —CX₃, —CN, —NO₂, —NR″C(═O)R″, —C(═O)R″, —C(═O)N(R″)₂, —S(═O)₂R″, —S(═O)₂NR″, —S(═O)R″, —OP(═O)(OR″)₂, —P(═O)(OR″)₂, —PO₃^{═}, PO₃H₂, —C(═O)R″, —C(═S)R″, —CO₂R″, —CO₂⁻, —C(═S)OR″, —C(═O)SR″, —C(═S)SR″, —C(═O)N(R″)₂, —C(═S)N(R″)₂, and —C(═NR)N(R″)₂, where each X is independently selected from the group consisting of a halogen: —F, —Cl, —Br, and —I; and wherein each R″ is independently selected from the group consisting of —H, —C₁-C₂₀ alkyl, —C₆-C₂₀ aryl, —C₃-C₁₄ heterocycle, a protecting group, and a prodrug moiety.

More typically substituents are selected from the group consisting of —X, —R″, —OH, —OR″, —SR″, —N(R″)₂, —N(R″)₃, ═NR″, —NR″C(═O)R″, —C(═O)R″, —C(═O)N(R″)₂, —S(═O)₂R″, —S(═O)₂NR″, —S(═O)R″, —C(═O)R″, —C(═S)R″, —C(═O)N(R″)₂, —C(═S)N(R″)₂, and —C(═NR)N(R″)₂, wherein each X is independently selected from the group consisting of —F and —Cl, or are selected from the group consisting of —X, —R″, —OH, —OR″, —N(R″)₂, —N(R″)₃, —NR″C(═O)R″, —C(═O)N(R″)₂, —S(═O)₂R″, —S(═O)₂NR″, —S(═O)R″, —C(═O)R″, —C(═O)N(R″)₂, —C(═NR)N(R″)₂, a protecting group, and a prodrug moiety, wherein each X is —F; and wherein each R′ is independently selected from the group consisting of hydrogen, —C₁-C₂₀ alkyl, —C₆-C₂₀ aryl, —C₃-C₁₄ heterocycle, a protecting group, and a prodrug moiety.

In some aspects, a substituent on an alkyl, alkenyl, or alkynyl is selected from the group consisting —N(R″)₂, —N(R″)₃ and —C(═NR)N(R″)₂, wherein R″ is selected from the group consisting of hydrogen and —C₁-C₂₀ alkyl. In other aspects, alkyl, alkenyl, or alkynyl is substituted with a series of ethyleneoxy moieties to define a PEG Unit as described herein. In some embodiments, alkylene, carbocycle, carbocyclo, arylene, heteroalkyl, heteroalkylene, heterocycle, heterocyclo, heteroaryl, and heteroarylene groups as described above are similarly substituted.

The term “unsubstituted” means that the specified group bears no substituents. Where the term “substituted is used to described a structural system, the substitution is meant to occur at any valency-allowed position on the system. When a group or moiety bears more than one substituent, it is understood that the substituents may be the same or different from one another. In some embodiments a substituted group or moiety bears from one to five substituents. In some embodiments a substituted group or moiety bears one substituent. In some embodiments a substituted group or moiety bears two substituents. In some embodiments a substituted group or moiety bears three substituents. In some embodiments a substituted group or moiety bears four substituents. In some embodiments a substituted group or moiety bears five substituents.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined herein. It will be understood by those skilled in the art, with respect to any group containing one or more substituents, that such groups are not intended to introduce any substitution or substitution patterns that are sterically impractical, synthetically non-feasible, and/or inherently unstable. It will also be understood that where a group or moiety is optionally substituted, the disclosure includes both embodiments in which the group or moiety is substituted and embodiments in which the group or moiety is unsubstituted.

“Protecting group” as used here means a moiety that prevents or reduces the ability of the atom or functional group to which it is linked from participating in unwanted reactions. Typical protecting groups for atoms or functional groups are given in Greene (1999), “PROTECTIVE GROUPS IN ORGANIC SYNTHESIS, 3^{RD} ED.”, Wiley Interscience, which is incorporated by reference herein. Protecting groups for heteroatoms such as oxygen, sulfur and nitrogen are used in some instances to minimize or avoid unwanted their reactions with electrophilic compounds. In other instances, the protecting group is used to reduce or eliminate the nucleophilicity and/or basicity of the unprotected heteroatom. Non-limiting examples of protected oxygen are given by —OR^{PR}, wherein R^{PR} is a protecting group for hydroxyl, wherein hydroxyl is typically protected as an ester (e.g. acetate, propionate or benzoate). Other protecting groups for hydroxyl avoid interfering with the nucleophilicity of organometallic reagents or other highly basic reagents, where hydroxyl is typically protected as an ether, including alkyl or heterocycloalkyl ethers, (e.g., methyl or tetrahydropyranyl ethers), alkoxymethyl ethers (e.g., methoxymethyl or ethoxymethyl ethers), optionally substituted aryl ethers, and silyl ethers (e.g., trimethylsilyl (TMS), triethylsilyl (TES), tert-butyldiphenylsilyl (TBDPS), tert-butyldimethylsilyl (TBS/TBDMS), triisopropylsilyl (TIPS) and [2-(trimethylsilyl)ethoxy]-methylsilyl (SEM)). Nitrogen protecting groups include those for primary or secondary amines as in —NHR^{PR} or —N(R^{PR})₂—, wherein least one of R^{PR} is a nitrogen atom protecting group or both R^{PR} together comprise a protecting group.

A protecting group is suitable when it is capable of preventing or avoiding unwanted side-reactions or premature loss of the protecting group under reaction conditions required to effect desired chemical transformation elsewhere in the molecule and during purification of the newly formed molecule when desired, and are removable under conditions that do not adversely affect the structure or stereochemical integrity of that newly formed molecule. By way of example and not limitation, a suitable protecting group includes those previously described for protecting otherwise reactive functional groups. A suitable protecting group is sometimes a protecting group used in peptide coupling reactions.

“Electron withdrawing group” as used herein means a functional group or electronegative atom that draws electron density away from an atom to which it is bonded either inductively and/or through resonance, whichever is more dominant (i.e., in some aspects, a functional group or atom is electron withdrawing inductively but is overall electron donating through resonance) and tends to stabilize anions or electron-rich moieties. The electron withdrawing effect is typically transmitted inductively, albeit in attenuated form, to other atoms attached to the bonded atom that has been made electron deficient by the electron withdrawing group (EWG), thus affecting the electrophilicity of a more remote reactive center. Exemplary electron withdrawing groups include, but are not limited to —C(═O), —CN, —NO₂, —CX₃, —X, —C(═O)OR′, —C(═O)N(R′)₂, —C(═O)R, —C(═O)X, —S(═O)₂R′, —S(═O)₂OR′, —S(═O)₂NHR′, —S(═O)₂N(R′)₂, —P(═O)(OR′)₂, —P(═O)(CH₃)NHR′, —NO, —N(R′)₃+, wherein X is —F, —Br, —Cl, or —I, and R′ in some aspects is, at each occurrence, independently selected from the group consisting of hydrogen and C₁₋₆ alkyl, and certain O-linked moieties as described herein such as acyloxy.

Exemplary EWGs can also include aryl groups (e.g., phenyl) depending on substitution of its aromatic ring and certain heteroaryl groups (e.g., pyridine). Thus, the term “electron withdrawing groups” also includes aryls or heteroaryls that are further substituted with electron withdrawing groups. Typically, electron withdrawing groups on aryls or heteroaryls are —C(═O), —CN, —NO₂, —CX₃, and —X, wherein X independently selected is halogen, typically —F or Cl. Depending on their substituents, an alkyl moiety may also be an electron withdrawing group.

“Succinimide moiety” as used herein refers to an organic moiety comprising a succinimide ring system, which is present in one type of Stretcher Unit (Z) that typically further comprises an alkylene-containing moiety bonded to the imide nitrogen of that ring system. A succinimide moiety typically results from Michael addition of a sulfhydryl group of a Ligand Unit to the maleimide ring system of a Stretcher Unit precursor (Z′) in a Drug Linker compound or maleimide-containing intermediate thereof. A succinimide moiety therefore comprises a thio-substituted succinimide ring system and when present in a Ligand-Drug Conjugate compound has its imide nitrogen substituted with the remainder of the Linker Unit of the Ligand-Drug Conjugate compound and is optionally substituted with substituent(s) that were present on the maleimide ring system of Z′.

“Succinic acid-amide moiety” as used herein refers to a succinic acid moiety wherein one of the two carboxylic acid groups is replaced with an amide substituent that results from the thio-substituted succinimide ring system of a succinimide moiety as defined herein having undergone breakage of one of its carbonyl-nitrogen bonds by hydrolysis. In some aspects, the succinic acid-amide moiety has the structure:

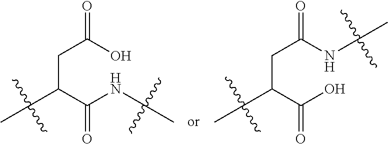 [SMILES: *NC(=O)C(*)CC(=O)O ; *NC(=O)CC(*)C(=O)O]

wherein the wavy line on the left indicates attachment to a Ligand Unit or hydrogen atom and the wavy line on the right indicates attachment to the remainder of a Ligand-Drug Conjugate compound, Drug-Linker compound, intermediate, or fragment thereof. Hydrolysis resulting in a succinic acid-amide moiety provides a Linker Unit less likely to suffer premature loss of the Ligand Unit to which it is bonded through elimination of the antibody-thio substituent. Hydrolysis of the succinimide ring system of the thio-substituted succinimide moiety is expected to provide regiochemical isomers of acid-amide moieties that are due to differences in reactivity of the two carbonyl carbons of the succinimide ring system attributable at least in part to any substituent present in the maleimide ring system of the Stretcher Unit precursor and to the thio substituent introduced by the targeting ligand, which is a precursor to the Ligand Unit.

In many instances, the assembly of the conjugates, linkers and components described herein will refer to reactive groups. A “reactive group” or RG is a group that contains a reactive site (RS) capable of forming a bond with either the components of the Linker Unit Q or the Drug Unit D. RS is the reactive site within a Reactive Group (RG). Reactive groups include sulfhydryl groups to form disulfide bonds or thioether bonds, aldehyde, ketone, or hydrazine groups to form hydrazone bonds, carboxylic or amino groups to form peptide bonds, carboxylic or hydroxy groups to form ester bonds, sulfonic acids to form sulfonamide bonds, alcohols to form carbamate bonds, and amines to form sulfonamide bonds or carbamate bonds.

The following table is illustrative of Reactive Groups, Reactive Sites, and exemplary functional groups capable of forming after reaction of the reactive site. The table is not limiting. One of skill in the art will appreciate that the noted R* and R** portions in the table are effectively any organic moiety (e.g., an alkyl group, aryl group, heteroaryl group, or substituted alkyl, aryl, or heteroaryl, group) that is compatible with the bond formation provided in converting RG to one of the Exemplary Functional Groups. It will also be appreciated that, as applied to the various aspects of the present invention, R* represents one or more components of the self-stabilizing linker or optional secondary linker, and R** represents one or more components of the optional secondary linker, Drug Unit, stabilizing unit, or detection unit.

| | | Exemplary Functional |
|---|---|---|
| RG | RS | Groups |
| 1) R*—SH | —S— | R*—S—R**, R*—S—S—R** |
| 2) R*—C(═O)OH | —C(═O)— | R*—C(═O)NH—R** |
| 3) R*—C(═O)ONHS | —C(═O)— | R*—C(═O)NH—R** |
| 4) R*S(═O)₂—OH | —S(═O)₂— | R*S(═O)₂NH—R** |
| 5) R*—CH₂—X | —CH₂— | R*—CH₂—S—R |
| (X is Br, I, Cl) | | |
| 6) R*—NH₂ | —N— | R*—NHC(═O)R** |

### II. Embodiments

### A. Auristatin Compounds

The present application is based, in part, on the surprising discovery that it is possible to improve the properties of auristatin-containing Ligand-Drug Conjugate compounds by tuning the hydrophilicity of auristatin drugs with polar moieities, e.g., hydroxyl groups. The hydrophobicity of auristatin drugs is a known barrier to their implementation in pharmaceuticals, including Ligand-Drug Conjugates, due to the potential for off-target toxicity due to high bystander activity and rapid clearance of the drug from the body of subjects. The compounds of the present application attenuate the hydphobicity of the Drug Unit, which in turn attenuates the off-target toxicity and rapid clearance of the drugs. The use of Drug Units with polar moieities in certain locations on the auristatin backbone leads to decreased permeability and on-cell potency of the free drug, and thus fewer off-target effects during treatment, without sacrificing efficacy of intact Ligand-Drug Conjugate compounds that incorporate the Drug Units.

In some embodiments herein, provided are auristatin compounds comprising at least one polar moiety on the auristatin backbone. In some embodiments, provided herein are Drug-Linker compounds comprising an auristatin moiety, as described herein. In some embodiments, provided herein are Ligand-Drug Conjugate compounds comprising an auristatin moiety, as described herein. In some embodiments, provided herein are methods of treating cancer using the Ligand-Drug Conjugate compounds described herein. In some embodiments, provided are methods of making auristatin compounds comprising at least one hydrophilic moiety, Drug-Linkers thereof and their intermediates, and Ligand-Drug Conjugate compounds thereof. In some embodiments, the polar moiety is a polar moiety other than carboxylate. In some embodiments, the polar moiety is a hydroxyl group.

In some embodiments, provided is a compound of formula (I):

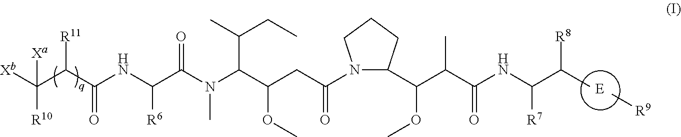

or a salt thereof, wherein
X^{b} is —NR¹R²; and X^{a} is

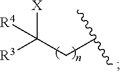

or
X^{a} and X^{b} are taken together with the carbon atom to which they are attached to form

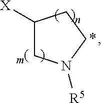

wherein the asterisk denotes the carbon atom of Formula (I) that bears the X^{a} and X^{b} groups;
R¹, R², R³, R⁴, R^{a}, R^{b}, R⁵, and R¹⁰ are each independently H or C₁-C₄ alkyl;
X is H, OH, —C(O)NR^{a}R^{b}, —S(O)₂R^{a}, —S(O)—R^{a} —S(O)₂NR^{a}R^{b}, —NHS(O)₂R^{a}, or —NHC(O)R^{a};
R⁶ is C₁-C₄ alkyl optionally substituted with OH;
R⁷ is H, C₁-C₄ alkyl optionally substituted with one or two OH moieties, or 5-6 membered heteroaryl;
E is phenyl or 5-6 membered heteroaryl;
R⁸, R⁹, and R¹¹ are each independently H or OH;
n is 0, 1, 2, or 3;
m is 1, 2, 3, or 4; and
q is 0 or 1,
wherein when X is H, at least two of R⁷, R⁸, R⁹, and R¹¹ comprise an OH moiety.

In some embodiments, provided is a compound of formula (Iz):

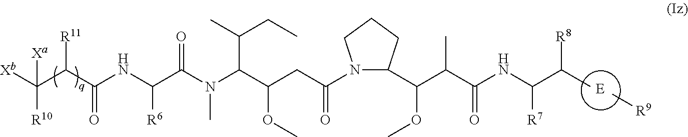

or a salt thereof, wherein
X^{b} is —NR¹R²; and X^{a} is

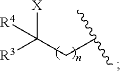

or
X^{a} and X^{b} are taken together with the carbon atom to which they are attached to form

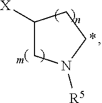

wherein the asterisk denotes the carbon atom of Formula (I) that bears the X^{a} and X^{b} groups;
R¹, R², R³, R⁴, R^{a}, R^{b}, R⁵, and R¹⁰ are each independently H or C₁-C₄ alkyl;
X is OH, —C(O)NR^{a}R^{b}, —S(O)₂R^{a}, —S(O)—R^{a} —S(O)₂NR^{a}R^{b}, —NHS(O)₂R^{a}, or —NHC(O)R^{a};
R⁶ is C₁-C₄ alkyl optionally substituted with OH;
R⁷ is H, C₁-C₄ alkyl optionally substituted with one or two OH moieties, or 5-6 membered heteroaryl;
E is phenyl or 5-6 membered heteroaryl;
R⁸, R⁹, and R¹¹ are each independently H or OH;
n is 0, 1, 2, or 3;
m is 1, 2, 3, or 4; and
q is 0 or 1.

In some embodiments, provided is compound of Formula (II):

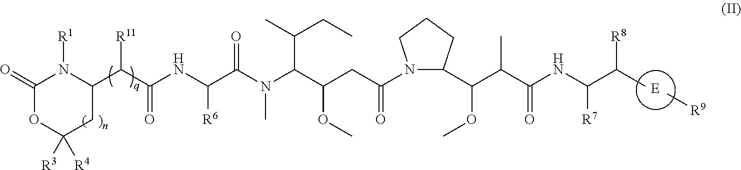

or a salt thereof, wherein
R¹, R³, and R⁴ are independently H or C₁-C₄ alkyl;
R⁶ is C₁-C₄ alkyl optionally substituted with OH;
R⁷ is H, C₁-C₄ alkyl optionally substituted with one or two OH moieties, or 5-6 membered heteroaryl;
E is phenyl or 5-6 membered heteroaryl;
R⁸, R⁹, and R¹¹ are each independently H or OH;
n is 0, 1, or 2; and
q is 0 or 1.

In some embodiments of Formula (I), q is 0. In some embodiments of Formula (I), q is 1. In some embodiments of Formula (II), q is 0. In some embodiments of Formula (II), q is 1.

In some embodiments of Formula (I) or (Iz), X^{b} is —NR¹R² is and X^{a} is

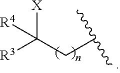

In some embodiments, R¹ and R² are each independently C₁-C₄ alkyl. In some embodiments, R¹ and R² are both H. In some embodiments, R¹ and R² are each independently n-propyl, isopropyl, ethyl, or methyl. In some embodiments, R¹ and R² are each independently ethyl or methyl. In some embodiments, R¹ and R² are both methyl. In some embodiments, R¹ is H and R² is C₁-C₄ alkyl. In some embodiments, R¹ is H and R² is n-propyl, isopropyl, ethyl, or methyl. In some embodiments, R¹ is H and R² is ethyl or methyl. In some embodiments, R¹ is H and R² is methyl. In some embodiments, R³ and R⁴ are each independently C₁-C₄ alkyl. In some embodiments, R³ is H and R⁴ is C₁-C₄ alkyl. In some embodiments, R³ and R⁴ are H. In some embodiments, R³ is H and R⁴ is methyl. In some embodiments, n is 0, 1, 2, or 3. In some embodiments, n is 0, 1, or 2. In some embodiments, n is 0 or 1. In some embodiments, n is 1. In some embodiments, n is 0. In some embodiments, R¹ is H, R² is methyl, R³ is H, R⁴ is H, and n is 0. In some embodiments, R¹ is methyl, R² is methyl, R³ is H, R⁴ is H, and n is 0.

In some embodiments of Formula (I) or (Iz), X^{a} and X^{b} are taken together with the carbon atom to which they are attached to form

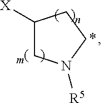

wherein the asterisk denotes the carbon atom of Formula (I) that bears the X^{a} and X^{b} groups. In some embodiments, R⁵ is H, n-propyl, isopropyl, ethyl, or methyl. In some embodiments, R⁵ is H, ethyl, or methyl. In some embodiments, R⁵ is H or methyl. In some embodiments, R⁵ is ethyl or methyl. In some embodiments, R⁵ is H. In some embodiments, R⁵ is ethyl. In some embodiments, R⁵ is methyl. In some embodiments, m is 1, 2, or 3. In some embodiments, m is 1 or 2. In some embodiments, m is 2 or 3. In some embodiments, m is 2. In some embodiments, n is 0, 1, or 2. In some embodiments, n is 0 or 1. In some embodiments, n is 0 or 1 and m is 0, 1, or 2. In some embodiments, n is 0 and m is 2. In some embodiments, n is 1 and m is 1. In some embodiments, m is 2 and n is 1. In some embodiments, X is H or OH. In some embodiments, X is H. In some embodiments, X is OH.

In some embodiments of Formula (I), X is H, OH, —C(O)NR^{a}R^{b}, —S(O)₂R^{a}, —S(O)—R^{a}, —S(O)₂NR^{a}R^{b}, —NHS(O)₂R^{a}, or —NHC(O)R^{a}. In some embodiments, X is OH, —S(O)₂R^{a}, —S(O)—R^{a}, —S(O)₂NR^{a}R^{b}, —NHS(O)₂R^{a}, or —NHC(O)R^{a}. In some embodiments, X is H or OH. In some embodiments, X is OH. In some embodiments, X is OH, —NHS(O)₂R^{a}, or —NHC(O)R^{a}. In some embodiments, X is OH, —S(O)₂R^{a}, —S(O)—R^{a}, or —S(O)₂NR^{a}R^{b}. In some embodiments, X is —NHS(O)₂R^{a}, or —NHC(O)R^{a}. In some embodiments, X is —S(O)₂R^{a}, —S(O)—R^{a}, or —S(O)₂NR^{a}R^{b}. In some embodiments, X is H, OH, —NHS(O)₂R^{a}, or —NHC(O)R^{a}. In some embodiments, X is H, OH, —S(O)₂R^{a}, —S(O)—R^{a}, or —S(O)₂NR^{a}R^{b}. In some embodiments, X is H, —NHS(O)₂R^{a}, or —NHC(O)R^{a}. In some embodiments, X is H, —S(O)₂R^{a}, —S(O)—R^{a}, or —S(O)₂NR^{a}R^{b}. In some embodiments, X is H, OH, or —C(O)NR^{a}R^{b}. In some embodiments, X is OH or —C(O)NR^{a}R^{b}. In some embodiments, X is H or —C(O)NR^{a}R^{b}. In some embodiments, R^{a} and R^{b} are independently H, n-propyl, isopropyl, ethyl, or methyl. In some embodiments, R^{a} and R^{b} are independently H, ethyl, or methyl. In some embodiments, R^{a} and R^{b} are independently H or methyl. In some embodiments, R^{a} is H and R^{b} is n-propyl, isopropyl, ethyl, or methyl. In some embodiments, R^{a} is H and R^{b} is methyl.

In some embodiments of Formula (Iz), X is OH, —C(O)NR^{a}R^{b}, —S(O)₂R^{a}, —S(O)—R^{a}, —S(O)₂NR^{a}R^{b}, —NHS(O)₂R^{a}, or —NHC(O)R^{a}. In some embodiments, X is OH. In some embodiments, X is OH, —NHS(O)₂R^{a}, or —NHC(O)R^{a}. In some embodiments, X is OH, —S(O)₂R^{a}, —S(O)—R^{a}, or —S(O)₂NR^{a}R^{b}. In some embodiments, X is —NHS(O)₂R^{a}, or —NHC(O)R^{a}. In some embodiments, X is —S(O)₂R^{a}, —S(O)—R^{a}, or —S(O)₂NR^{a}R^{b}. In some embodiments, X is OH or —C(O)NR^{a}R^{b}. In some embodiments, X is —C(O)NR^{a}R^{b}. In some embodiments, R^{a} and R^{b} are independently H, n-propyl, isopropyl, ethyl, or methyl. In some embodiments, R^{a} and R^{b} are independently H, ethyl, or methyl. In some embodiments, R^{a} and R^{b} are independently H or methyl. In some embodiments, R^{a} is H and R^{b} is n-propyl, isopropyl, ethyl, or methyl. In some embodiments, R^{a} is H and R^{b} is methyl.

In some embodiments of Formula (I) or (Iz), X^{b} is —NR¹R² is and X^{a} is

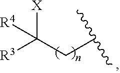

X is OH, R¹ is H or methyl, R² is methyl, R³ is H, R⁴ is H, and n is 0. In some embodiments, X is OH, R¹ is methyl, R² is methyl, R³ is H, R⁴ is H, and n is 0. In some embodiments, X is OH, R¹ is H, R² is methyl, R³ is H, R⁴ is H, and n is 0. In some embodiments, X is OH, R³ and R⁴ are H, and n is 0. In some embodiments, X is OH, R¹ is H, R² is H, R³ is H, R⁴ is H, and n is 0.

In some embodiments of Formula (II), R¹ is H, n-propyl, isopropyl, ethyl, or methyl. In some embodiments, R¹ is H, ethyl, or methyl. In some embodiments, R¹ is H or methyl. In some embodiments, R¹ is ethyl. In some embodiments, R¹ is methyl. In some embodiments, R¹ is H. In some embodiments, n is 0 or 1. In some embodiments, n is 1. In some embodiments, n is 0.

In some embodiments of Formula (II), R³ and R⁴ are H. In some embodiments, R³ and R⁴ are methyl. In some embodiments, R³ is H and R⁴ is methyl.

In some embodiments or Formula (I), (Iz), or (II), R⁶ is n-propyl, isopropyl, ethyl, or methyl. In some embodiments, R⁶ is ethyl or methyl. In some embodiments, R⁶ is isopropyl. In some embodiments, R⁶ is n-propyl, isopropyl, ethyl, or methyl, each of which is substituted with OH. In some embodiments, R⁶ is isopropyl substituted with OH. In some embodiments, R⁶ is ethyl substituted with OH. In some embodiments, R⁶ is methyl substituted with OH.

In some embodiments of Formula (I), (Iz), or (II), R⁷ is C₁-C₄ alkyl substituted with OH. In some embodiments, R⁷ is C₁-C₄ alkyl substituted by two OH moieties. In some embodiments, R⁷ is n-propyl, isopropyl, ethyl, or methyl, each of which is substituted with OH. In some embodiments, R⁷ is C₁-C₄ alkyl. In some embodiments, R⁷ is n-propyl, isopropyl, ethyl, or methyl. In some embodiments, R⁷ is methyl. In some embodiments, R⁷ is —CH₂CH₂OH or CH₂OH. In some embodiments, R⁷ is —CH₂OH. In some embodiments, R⁷ is 5-6 membered heteroaryl. In some embodiments, R⁷ is 5-membered heteroaryl. In some embodiments, R⁷ is thiazolyl.

In some embodiments of Formula (I), (Iz), or (II), R⁸, R⁹, and R¹¹ are H. In some embodiments, R⁸ is OH, R⁹ is H, and R¹¹ is H. In some embodiments, R⁸ and R⁹ are each OH and R¹¹ is H. In some embodiments, R⁸ is OH, R⁹ is H, and R¹¹ is OH. In some embodiments, R⁸ is H, R⁹ is H, and R¹¹ is OH.

In some embodiments of Formula (I), (Iz), or (II), E is a 6-membered ring. In some embodiments, E is pyridine or phenyl. In some embodiments, E is phenyl.

In some embodiments of Formula (I), (Iz), or (II), R⁷ is —CH₂OH, R⁸ is H, R⁹ is H, and E is phenyl.

In some embodiments, provided is a compound of Formula (Ia):

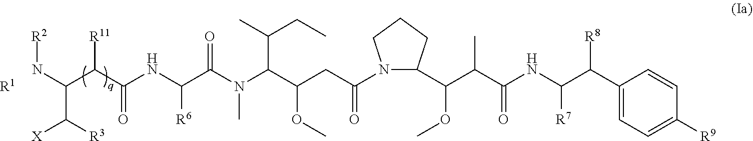

or a salt thereof, wherein
R¹, R², R³, R⁴, R^{a}, and R^{b} are each independently H or C₁-C₄ alkyl;
X is H, OH, —C(O)NR^{a}R^{b}, —S(O)₂R^{a}, —S(O)—R^{a} —S(O)₂NR^{a}R^{b}, —NHS(O)₂R^{a}, or —NHC(O)R^{a};
R⁶ is C₁-C₄ alkyl;
R⁷ is H, C₁-C₄ alkyl optionally substituted with one or two OH moieties, or 5-6 membered heteroaryl;
R⁸, R⁹, and R¹¹ are each independently H or OH;
q is 0 or 1; and
wherein when X is H, at least two of R⁷, R⁸, R⁹, and R¹¹ comprise an OH moiety.

In some embodiments of Formula (Ia), q is 0. In some embodiments, q is 1.

In some embodiments of Formula (Ia), R¹ is H and R² is C₁-C₄-alkyl. In some embodiments, R¹ is H and R² is n-propyl, isopropyl, ethyl, or methyl. In some embodiments, R¹ is H and R² is ethyl or methyl. In some embodiments, R¹ is H and R² is methyl. In some embodiments, R¹ and R² are each independently C₁-C₄ alkyl. In some embodiments, R¹ and R² are each independently n-propyl, isopropyl, ethyl, or methyl. In some embodiments, R¹ and R² are each independently ethyl or methyl. In some embodiments, R¹ and R² are methyl. In some embodiments, R¹ and R² are both H.

In some embodiments of Formula (Ia), X is H, OH, —C(O)NR^{a}R^{b}, —S(O)₂R^{a}, —S(O)—R^{a}, —S(O)₂NR^{a}R^{b}, —NHS(O)₂R^{a}, or —NHC(O)R^{a}. In some embodiments, X is OH, —S(O)₂R^{a}, —S(O)—R^{a}, —S(O)₂NR^{a}R^{b}, —NHS(O)₂R^{a}, or —NHC(O)R^{a}. In some embodiments, X is H or OH. In some embodiments, X is OH. In some embodiments, X is OH, —NHS(O)₂R^{a}, or —NHC(O)R^{a}. In some embodiments, X is OH, —S(O)₂R^{a}, —S(O)—R^{a}, or —S(O)₂NR^{a}R^{b}. In some embodiments, X is —NHS(O)₂R^{a}, or —NHC(O)R^{a}. In some embodiments, X is —S(O)₂R^{a}, —S(O)—R^{a}, or —S(O)₂NR^{a}R^{b}. In some embodiments, X is H, OH, —NHS(O)₂R^{a}, or —NHC(O)R^{a}. In some embodiments, X is H, OH, —S(O)₂R^{a}, —S(O)—R^{a}, or —S(O)₂NR^{a}R^{b}. In some embodiments, X is H, —NHS(O)₂R^{a}, or —NHC(O)R^{a}. In some embodiments, X is H, —S(O)₂R^{a}, —S(O)—R^{a}, or —S(O)₂NR^{a}R^{b}. In some embodiments, X is H, OH, or —C(O)NR^{a}R^{b}. In some embodiments, X is OH or —C(O)NR^{a}R^{b}. In some embodiments, X is H or —C(O)NR^{a}R^{b}. In some embodiments, R^{a} and R^{b} are independently H, n-propyl, isopropyl, ethyl, or methyl. In some embodiments, R^{a} and R^{b} are independently H, ethyl, or methyl. In some embodiments, R^{a} and R^{b} are independently H or methyl. In some embodiments, R^{a} is H and R^{b} is n-propyl, isopropyl, ethyl, or methyl. In some embodiments, R^{a} is H and R^{b} is methyl.

In some embodiments of Formula (Ia), X is OH, —C(O)NR^{a}R^{b}, —S(O)₂R^{a}, —S(O)—R^{a}, —S(O)₂NR^{a}R^{b}, —NHS(O)₂R^{a}, or —NHC(O)R^{a}. In some embodiments, X is OH. In some embodiments, X is OH, —NHS(O)₂R^{a}, or —NHC(O)R^{a}. In some embodiments, X is OH, —S(O)₂R^{a}, —S(O)—R^{a}, or —S(O)₂NR^{a}R^{b}. In some embodiments, X is —NHS(O)₂R^{a}, or —NHC(O)R^{a}. In some embodiments, X is —S(O)₂R^{a}, —S(O)—R^{a}, or —S(O)₂NR^{a}R^{b}. In some embodiments, X is OH or —C(O)NR^{a}R^{b}. In some embodiments, X is —C(O)NR^{a}R^{b}. In some embodiments, R^{a} and R^{b} are independently H, n-propyl, isopropyl, ethyl, or methyl. In some embodiments, R^{a} and R^{b} are independently H, ethyl, or methyl. In some embodiments, R^{a} and R^{b} are independently H or methyl. In some embodiments, R^{a} is H and R^{b} is n-propyl, isopropyl, ethyl, or methyl. In some embodiments, R^{a} is H and R^{b} is methyl.

In some embodiments of Formula (Ia), R⁶ is n-propyl, isopropyl, ethyl, or methyl. In some embodiments, R⁶ is ethyl or methyl. In some embodiments, R⁶ is isopropyl. In some embodiments, R⁶ is n-propyl, isopropyl, ethyl, or methyl, each of which is substituted with OH. In some embodiments, R⁶ is isopropyl substituted with OH. In some embodiments, R⁶ is ethyl substituted with OH. In some embodiments, R⁶ is methyl substituted with OH.

In some embodiments of Formula (Ia), R⁷ is C₁-C₄ alkyl substituted with OH. In some embodiments, R⁷ is n-propyl, isopropyl, ethyl, or methyl, each of which is substituted with OH. In some embodiments, R⁷ is —CH₂CH₂OH or CH₂OH. In some embodiments, R⁷ is —CH₂OH. In some embodiments, R⁷ is C₁-C₄ alkyl. In some embodiments, R⁷ is n-propyl, isopropyl, ethyl, or methyl. In some embodiments, R⁷ is methyl. In some embodiments, R⁷ is 5-6 membered heteroaryl. In some embodiments, R⁷ is 5-membered heteroaryl. In some embodiments, R⁷ is thiazolyl.

In some embodiments of Formula (Ia), R⁸, R⁹, and R¹¹ are H. In some embodiments, R⁸ is OH, R⁹ is H, and R¹¹ is H. In some embodiments, R⁸ and R⁹ are each OH and R¹¹ is H. In some embodiments, R⁸ is OH, R⁹ is H, and R¹¹ is OH.

In some embodiments of Formula (Ia), X is OH, R³ is H or methyl, R¹ is H or C₁-C₄ alkyl, R² is C₁-C₄ alkyl, R₆ is C₁-C₄ alkyl, R⁷ is C₁-C₄ alkyl optionally substituted with OH, R⁸ is H or C₁-C₄ alkyl, R⁹ is H, and q is 0. In some embodiments, X is OH, R³ is H, R¹ is H or methyl, R² is methyl, R⁶ is isopropyl, R⁷ is CH₂OH, R⁸ is H, R⁹ is H, and q is 0.

In some embodiments, provided is a compound of Formula (Ib):

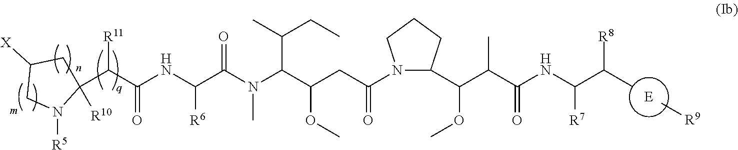

or a salt thereof, wherein the variables are as defined for Formula (I).

In some embodiments of Formula (Ib), X is H. In some embodiments, X is H or OH. In some embodiments, R⁵ is H or methyl. In some embodiments, R⁵ is H. In some embodiments, m is 1 or 2. In some embodiments, m is 2. In some embodiments, n is 2. In some embodiments, n is 0 or 1. In some embodiments, n is 1. In some embodiments, n is 0. In some embodiments, m is 2 and n is 0. In some embodiments, m is 1 and n is 1.

In some embodiments or Formula (Ib), R⁶ is n-propyl, isopropyl, ethyl, or methyl. In some embodiments, R⁶ is ethyl or methyl. In some embodiments, R⁶ is isopropyl. In some embodiments, R⁶ is n-propyl, isopropyl, ethyl, or methyl, each of which is substituted with OH. In some embodiments, R⁶ is isopropyl substituted with OH. In some embodiments, R⁶ is ethyl substituted with OH. In some embodiments, R⁶ is methyl substituted with OH.

In some embodiments of Formula (Ib), R⁷ is C₁-C₄ alkyl substituted with OH. In some embodiments, R⁷ is n-propyl, isopropyl, ethyl, or methyl, each of which substituted with OH. In some embodiments, R⁷ is C₁-C₄ alkyl. In some embodiments, R⁷ is n-propyl, isopropyl, ethyl, or methyl. In some embodiments, R⁷ is methyl. In some embodiments, R⁷ is —CH₂CH₂OH or CH₂OH. In some embodiments, R⁷ is —CH₂OH. In some embodiments, R⁷ is 5-6 membered heteroaryl. In some embodiments, R⁷ is 5-membered heteroaryl. In some embodiments, R⁷ is thiazolyl.

In some embodiments of Formula (Ib), R⁸, R⁹, and R¹¹ are each H. In some embodiments, R⁸ is OH, R⁹ is H, and R¹¹ is H. In some embodiments, R⁸ and R⁹ are each OH and R¹¹ is H. In some embodiments, R⁸ is OH, R⁹ is H, and R¹¹ is OH. In some embodiments, R⁸ is H, R⁹ is H, and R¹¹ is OH.

In some embodiments of Formula (Ib), E is a 6-membered ring. In some embodiments, E is pyridine or phenyl. In some embodiments, E is phenyl.

In some embodiments of Formula (Ib), R⁷ is —CH₂OH, R⁸ is H, R⁹ is H, and E is phenyl.

In some embodiments of Formula (Ib), R⁷ is —CH₂OH, R⁸ is H, and R⁹ is H. In some embodiments, m is 2, n is 0, X is H, R¹⁰ is methyl, R⁶ is isopropyl, R⁷ is H, R⁸ is OH, and R⁹ is OH. In some embodiments, m is 1, n is 1, X is H, R¹⁰ is methyl, R⁶ is isopropyl, R⁷ is H, R⁸ is OH, and R⁹ is OH. In some embodiments, m is 2, n is 1, X is H, R¹⁰ is methyl, R⁶ is isopropyl, R⁷ is H, R⁸ is OH, and R⁹ is OH.

In some embodiments, provided is a compound of Formula (Ic):

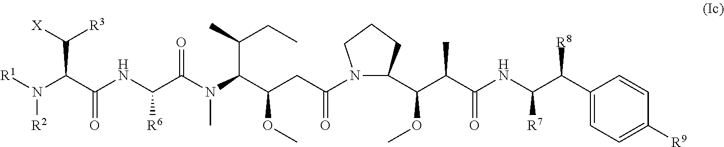

or a salt thereof, wherein the variables are as defined for Formula (I), (Iz), or (Ia).

In some embodiments, provided is a compound of Formula (Id):

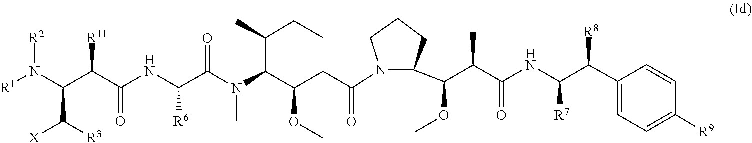

or a salt thereof, wherein the variables are as defined for Formula (I), (Iz), or (Ia).

In some embodiments, provided is a compound of Formula (Ie):

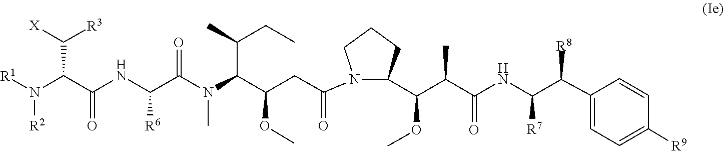

or a salt thereof, wherein the variables are as defined for Formula (I), (Iz), or (Ia).

In some embodiments, provided is a compound of Formula (Id):

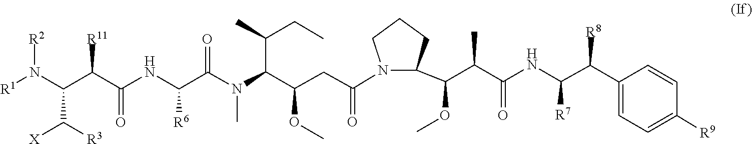

or a salt thereof, wherein the variables are as defined for Formula (I), (Iz), or (Ia).

In some embodiments of Formula (Ic), (Id), (Ie), or (If), R¹ and R¹ are both H. R¹ is H and R² is C₁-C₄-alkyl. In some embodiments, R¹ is H and R² is n-propyl, isopropyl, ethyl, or methyl. In some embodiments, R¹ is H and R² is ethyl or methyl. In some embodiments, R¹ is H and R² is methyl. In some embodiments, R¹ and R² are each independently C₁-C₄ alkyl. In some embodiments, R¹ and R² are each independently n-propyl, isopropyl, ethyl, or methyl. In some embodiments, R¹ and R² are each independently ethyl or methyl. In some embodiments, R¹ and R² are methyl.

In some embodiments of Formula (Ic), (Id), (Ie), or (If), X is H, OH, —C(O)NR^{a}R^{b}, or —NHC(O)R^{a}. In some embodiments, X is H, OH, —C(O)NR^{a}R^{b}, or —NHC(O)R^{a}. In some embodiments, X is H or OH. In some embodiments, X is OH. In some embodiments, R³ is H or C₁-C₄ alkyl. In some embodiments, R³ is H, n-propyl, isopropyl, ethyl, or methyl. In some embodiments, R³ is H, ethyl, or methyl. In some embodiments, X is H or OH and R³ is H or methyl. In some embodiments, X is OH and R³ is H. In some embodiments, X is OH and R³ is methyl. In some embodiments, R^{a} is ethyl or methyl. In some embodiments, R^{a} is methyl.

In some embodiments of Formula (Ic), (Id), (Ie), or (If), R⁶ is n-propyl, isopropyl, ethyl, or methyl. In some embodiments, R⁶ is ethyl or methyl. In some embodiments, R⁶ is isopropyl. In some embodiments, R⁶ is n-propyl, isopropyl, ethyl, or methyl, each of which is substituted with OH. In some embodiments, R⁶ is isopropyl substituted with OH. In some embodiments, R⁶ is ethyl substituted with OH. In some embodiments, R⁶ is methyl substituted with OH.

In some embodiments of Formula (Ic), (Id), (Ie), or (If), R⁷ is C₁-C₄ alkyl substituted with OH. In some embodiments, R⁷ is C₁-C₄ alkyl substituted by two OH moieties. In some embodiments, R⁷ is n-propyl, isopropyl, ethyl, or methyl, each of which is substituted with OH. In some embodiments, R⁷ is —CH₂CH₂OH or CH₂OH. In some embodiments, R⁷ is —CH₂OH. In some embodiments, R⁷ is C₁-C₄ alkyl. In some embodiments, R⁷ is n-propyl, isopropyl, ethyl, or methyl. In some embodiments, R⁷ is methyl. In some embodiments, R⁷ is 5-6 membered heteroaryl. In some embodiments, R⁷ is 5-membered heteroaryl. In some embodiments, R⁷ is thiazolyl.

In some embodiments of Formula (Ic) or (Ie), R⁸ and R⁹ are H. In some embodiments, R⁸ is OH and R⁹ is H. In some embodiments, R⁸ and R⁹ are each OH.

In some embodiments of Formula (Id) or (If), R⁸, R⁹, and R¹¹ are H. In some embodiments, R⁸ is OH, R⁹ is H, and R¹¹ is H. In some embodiments, R⁸ and R⁹ are each OH and R¹¹ is H. In some embodiments, R⁸ is OH, R⁹ is H, and R¹¹ is OH. In some embodiments, R⁸ is H, R⁹ is H, and R¹¹ is OH.

In some embodiments of Formula (Ic), (Id), (Ie), or (If), X is OH, R³ is H or methyl, R¹ is H or C₁-C₄ alkyl, R² is C₁-C₄ alkyl, R₆ is C₁-C₄ alkyl, R⁷ is C₁-C₄ alkyl optionally substituted with OH, R⁸ is H or C₁-C₄ alkyl, and R⁹ is H. In some embodiments, X is OH, R³ is H, R¹ is H or methyl, R² is methyl, R⁶ is isopropyl, R⁷ is CH₂OH, R⁸ is H, and R⁹ is H.

In some embodiments of Formula (Ic), R⁷ is —CH₂OH, R⁸ is H, and R⁹ is H.

In some embodiments, provided is a compound of Formula (IIa):

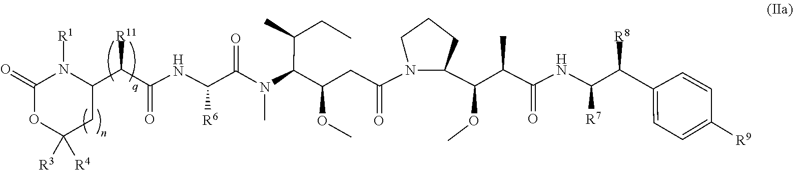

or a salt thereof, wherein the variables are as defined for Formula (II).

In some embodiments of Formula (IIa), q is 0. In some embodiments, q is 1.

In some embodiments of Formula (IIa), R¹ is H, n-propyl, isopropyl, ethyl, or methyl. In some embodiments, R¹ is H, ethyl, or methyl. In some embodiments, R¹ is H or methyl. In some embodiments, R¹ is ethyl. In some embodiments, R¹ is methyl. In some embodiments, R¹ is H. In some embodiments, n is 0 or 1. In some embodiments, n is 1. In some embodiments, n is 0.

In some embodiments or Formula (IIa), R⁶ is n-propyl, isopropyl, ethyl, or methyl. In some embodiments, R⁶ is ethyl or methyl. In some embodiments, R⁶ is isopropyl. In some embodiments, R⁶ is n-propyl, isopropyl, ethyl, or methyl, each of which is substituted with OH. In some embodiments, R⁶ is isopropyl substituted with OH. In some embodiments, R⁶ is ethyl substituted with OH. In some embodiments, R⁶ is methyl substituted with OH.

In some embodiments of Formula (IIa), R⁷ is C₁-C₄ alkyl substituted with OH. In some embodiments, R⁷ is C₁-C₄ alkyl substituted by two OH moieties. In some embodiments, R⁷ is n-propyl, isopropyl, ethyl, or methyl, each of which is substituted with OH. In some embodiments, R⁷ is C₁-C₄ alkyl. In some embodiments, R⁷ is n-propyl, isopropyl, ethyl, or methyl. In some embodiments, R⁷ is methyl. In some embodiments, R⁷ is —CH₂CH₂OH or CH₂OH. In some embodiments, R⁷ is —CH₂OH. In some embodiments, R⁷ is 5-6 membered heteroaryl. In some embodiments, R⁷ is 5-membered heteroaryl. In some embodiments, R⁷ is thiazolyl.

In some embodiments of Formula (IIa), R⁸, R⁹, and R¹¹ are H. In some embodiments, R⁸ is OH, R⁹ is H, and R¹¹ is H. In some embodiments, R⁸ and R⁹ are each OH and R¹¹ is H. In some embodiments, R⁸ is OH, R⁹ is H, and R¹¹ is OH. In some embodiments, R⁸ is H, R⁹ is H, and R¹¹ is OH.

In some embodiments of Formula (IIa), E is a 6-membered ring. In some embodiments, E is pyridine or phenyl. In some embodiments, E is phenyl.

In some embodiments of Formula (IIa), R⁷ is —CH₂OH, R⁸ is H, R⁹ is H, and R¹¹ is H.

In some embodiments, provided is a compound of Table 1, or a salt thereof (e.g., a pharmaceutically acceptable salt).

TABLE 1
Hydrophilic auristatin compounds

| Compound # | Compound Structure |
|---|---|
| 1.1 | 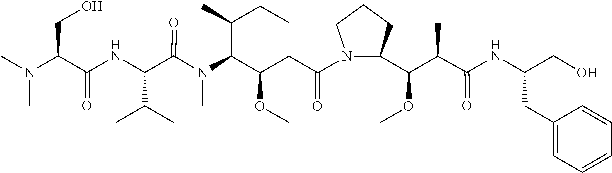 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)N(C)C)C(C)C] |
| 1.2 | 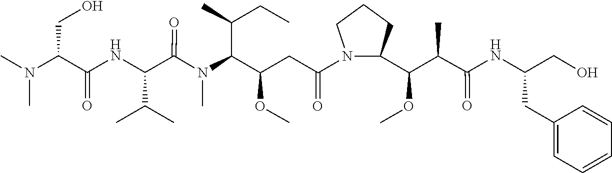 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](CO)N(C)C)C(C)C] |
| 1.3 | 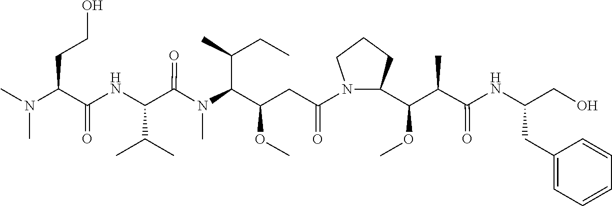 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CCO)N(C)C)C(C)C] |
| 1.4 | 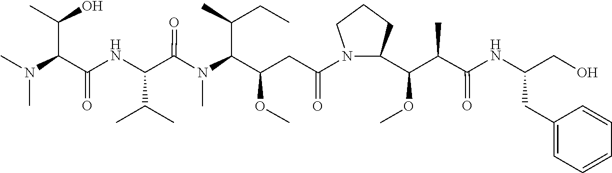 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H]([C@@H](C)O)N(C)C)C(C)C] |
| 1.5 | 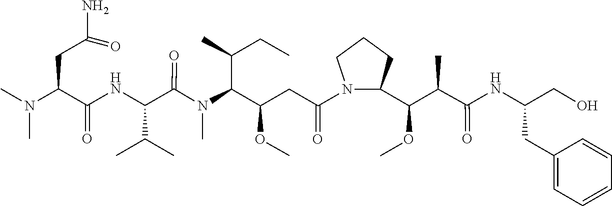 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CC(N)=O)N(C)C)C(C)C] |
| 1.6 | 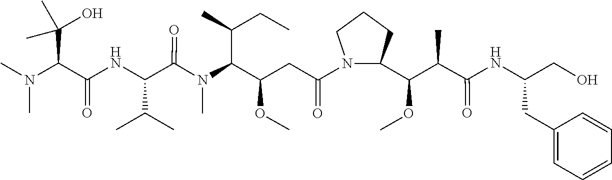 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](N(C)C)C(C)(C)O)C(C)C] |
| 1.7 | 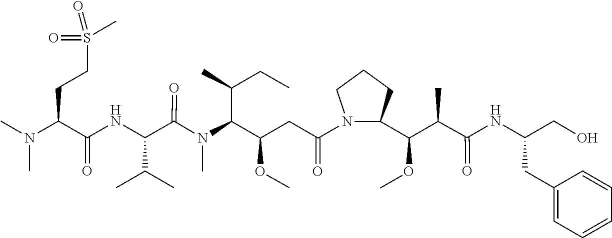 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CCS(C)(=O)=O)N(C)C)C(C)C] |
| 1.8 | 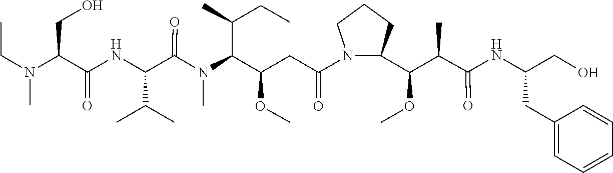 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)N(C)CC)C(C)C] |
| 1.9 | 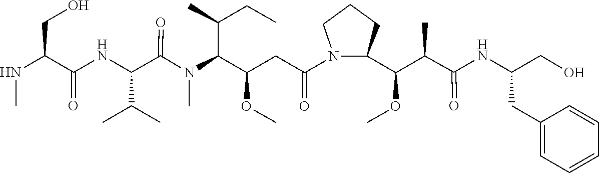 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)NC)C(C)C] |
| 1.10 | 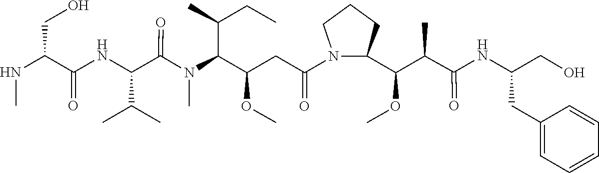 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](CO)NC)C(C)C] |
| 1.11 | 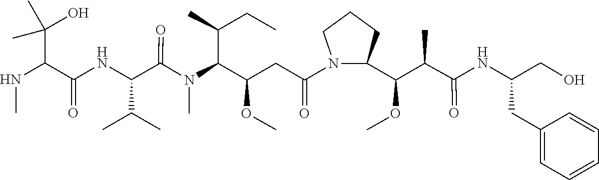 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)C(NC)C(C)(C)O)C(C)C] |
| 1.12 | 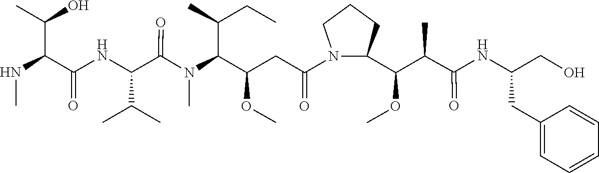 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](NC)[C@@H](C)O)C(C)C] |
| 1.13 | 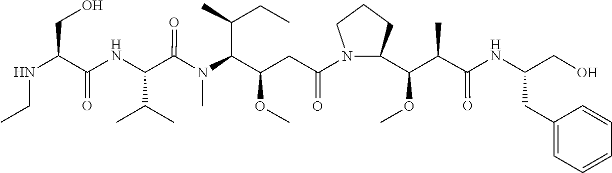 [SMILES: CCN[C@@H](CO)C(=O)N[C@H](C(=O)N(C)[C@@H]([C@@H](C)CC)[C@@H](CC(=O)N1CCC[C@H]1[C@H](OC)[C@@H](C)C(=O)N[C@H](CO)Cc1ccccc1)OC)C(C)C] |
| 1.14 | 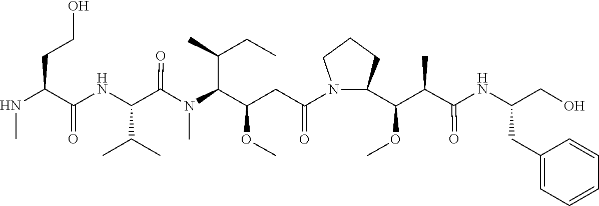 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CCO)NC)C(C)C] |
| 1.15 | 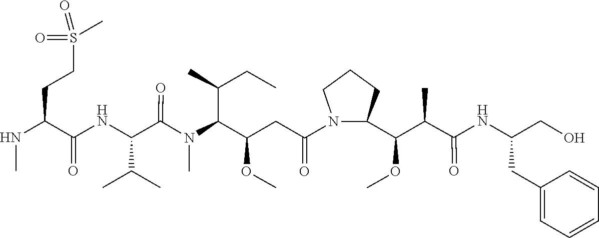 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CCS(C)(=O)=O)NC)C(C)C] |
| 1.16 | 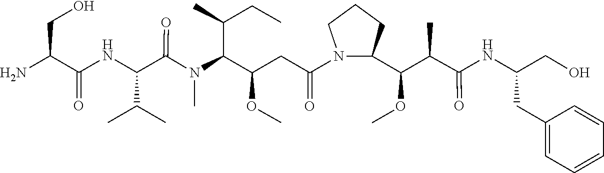 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](N)CO)C(C)C] |
| 1.17 | 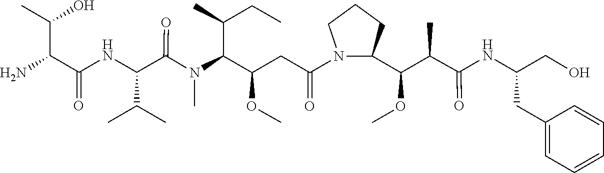 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](N)[C@H](C)O)C(C)C] |
| 1.18 | 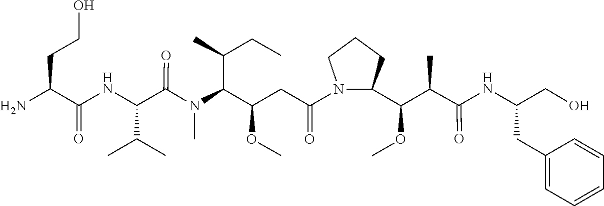 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](N)CCO)C(C)C] |
| 1.19 | 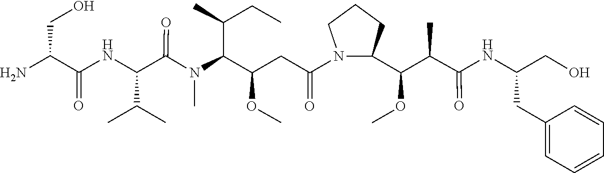 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](N)CO)C(C)C] |
| 1.20 | 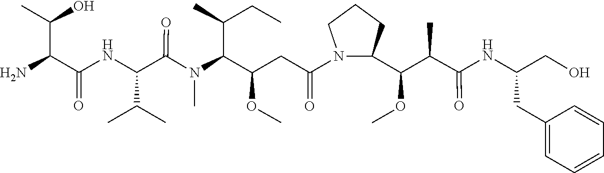 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](N)[C@@H](C)O)C(C)C] |
| 1.21 | 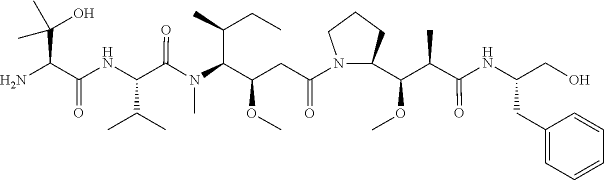 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](N)C(C)(C)O)C(C)C] |
| 1.22 | 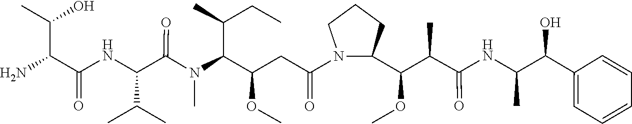 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](N)[C@H](C)O)C(C)C] |
| 1.23 | 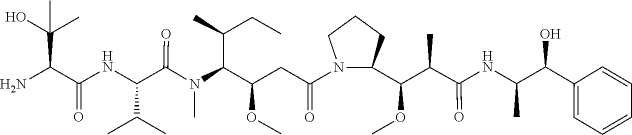 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](N)C(C)(C)O)C(C)C] |
| 1.24 | 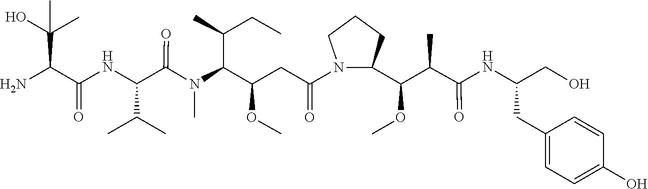 [SMILES: CC[C@H](C)[C@@H]([C@@H](CC(=O)N1CCC[C@H]1[C@H](OC)[C@@H](C)C(=O)N[C@H](CO)Cc1ccc(O)cc1)OC)N(C)C(=O)[C@@H](NC(=O)[C@@H](N)C(C)(C)O)C(C)C] |
| 1.25 | 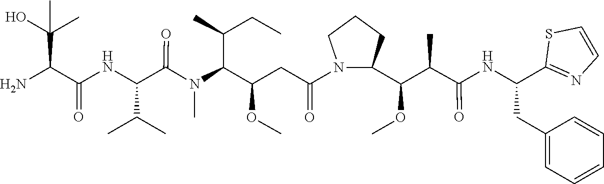 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H](N)C(C)(C)O)C(C)C] |
| 1.26 | 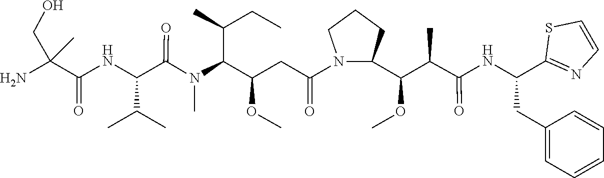 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)C(C)(N)CO)C(C)C] |
| 1.27 | 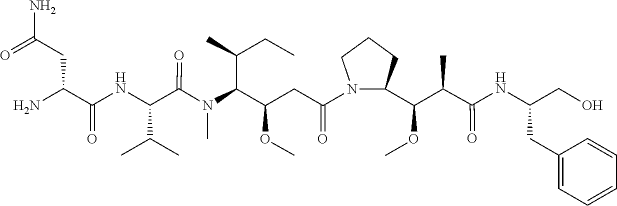 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](N)CC(N)=O)C(C)C] |
| 1.28 | 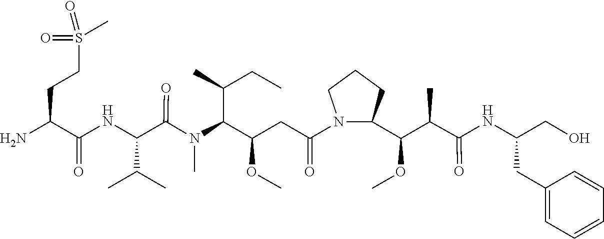 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](N)CCS(C)(=O)=O)C(C)C] |
| 1.29 | 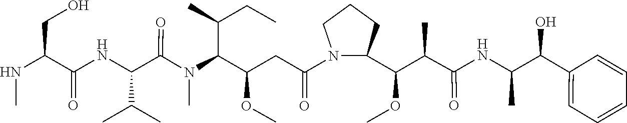 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](CO)NC)C(C)C] |
| 1.30 | 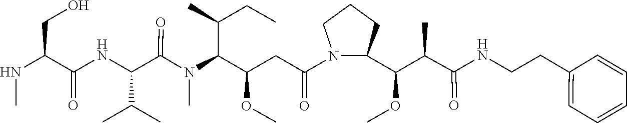 [SMILES: CC[C@H](C)[C@@H]([C@@H](CC(=O)N1CCC[C@H]1[C@H](OC)[C@@H](C)C(=O)NCCc1ccccc1)OC)N(C)C(=O)[C@@H](NC(=O)[C@H](CO)NC)C(C)C] |
| 1.31 | 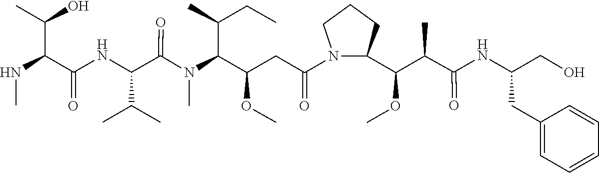 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](NC)[C@@H](C)O)C(C)C] |
| 1.32 | 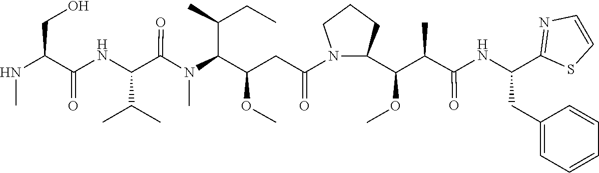 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H](CO)NC)C(C)C] |
| 1.33 | 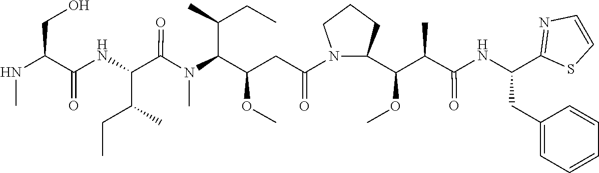 [SMILES: CC[C@@H](C)[C@H](NC(=O)[C@H](CO)NC)C(=O)N(C)[C@@H]([C@@H](C)CC)[C@@H](CC(=O)N1CCC[C@H]1[C@H](OC)[C@@H](C)C(=O)N[C@@H](Cc1ccccc1)c1nccs1)OC] |
| 1.34 | 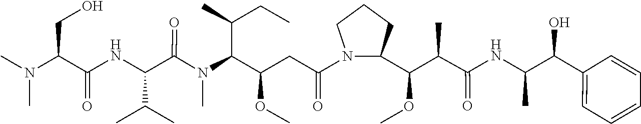 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](CO)N(C)C)C(C)C] |
| 1.35 | 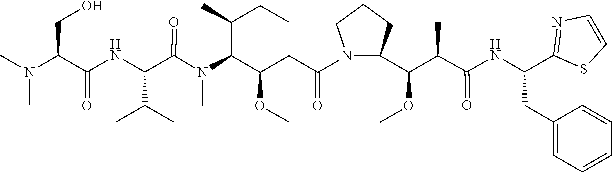 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H](CO)N(C)C)C(C)C] |
| 1.36 | 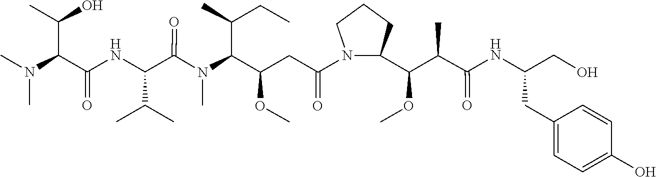 [SMILES: CC[C@H](C)[C@@H]([C@@H](CC(=O)N1CCC[C@H]1[C@H](OC)[C@@H](C)C(=O)N[C@H](CO)Cc1ccc(O)cc1)OC)N(C)C(=O)[C@@H](NC(=O)[C@H]([C@@H](C)O)N(C)C)C(C)C] |
| 1.37 | 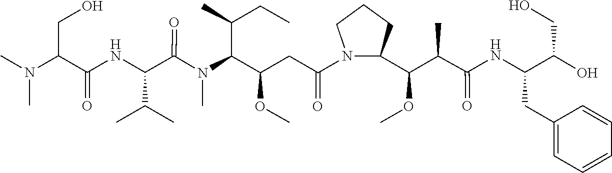 [SMILES: CC[C@H](C)[C@@H]([C@@H](CC(=O)N1CCC[C@H]1[C@H](OC)[C@@H](C)C(=O)N[C@@H](Cc1ccccc1)[C@@H](O)CO)OC)N(C)C(=O)[C@@H](NC(=O)C(CO)N(C)C)C(C)C] |
| 1.38 | 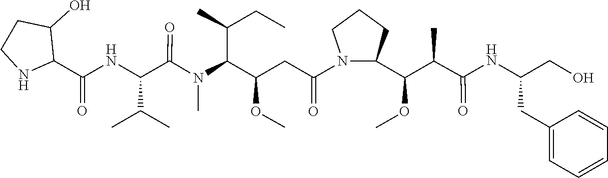 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)C1NCCC1O)C(C)C] |
| 1.39 | 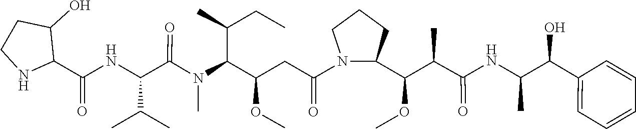 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)C1NCCC1O)C(C)C] |
| 1.40 | 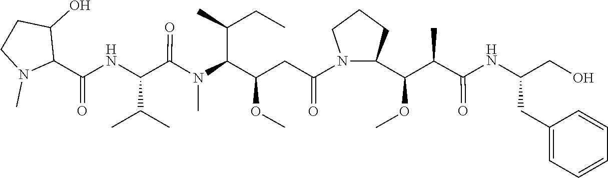 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)C1C(O)CCN1C)C(C)C] |
| 1.41 | 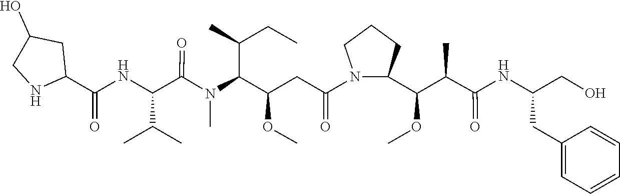 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)C1CC(O)CN1)C(C)C] |
| 1.42 | 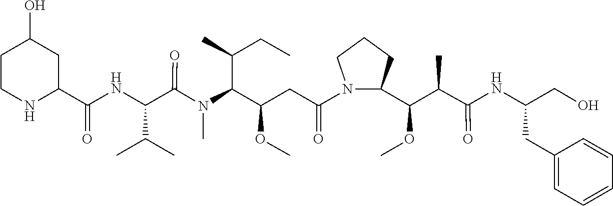 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)C1CC(O)CCN1)C(C)C] |
| 1.43 | 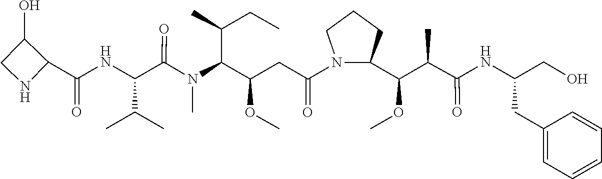 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)C1NCC1O)C(C)C] |
| 1.44 | 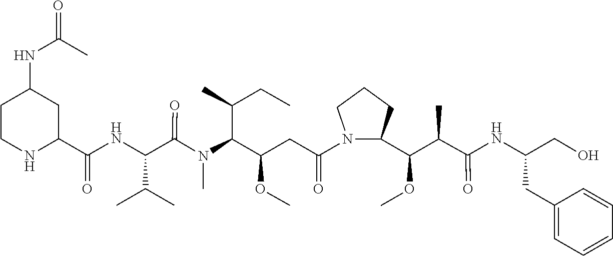 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)C1CC(NC(C)=O)CCN1)C(C)C] |
| 1.45 | 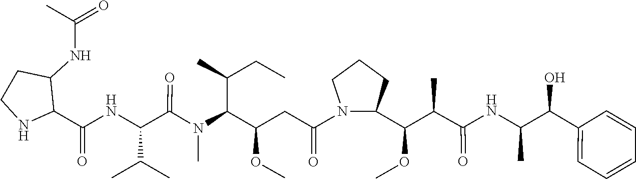 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)C1NCCC1NC(C)=O)C(C)C] |
| 1.46 | 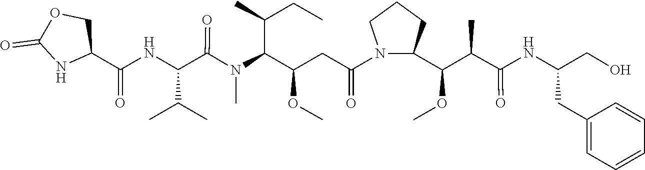 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H]1COC(=O)N1)C(C)C] |
| 1.47 | 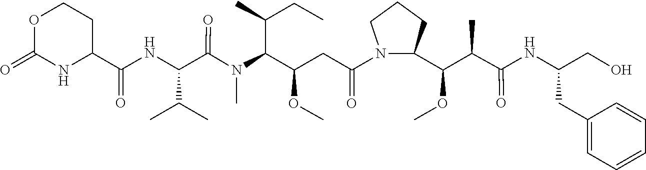 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)C1CCOC(=O)N1)C(C)C] |
| 1.48 | 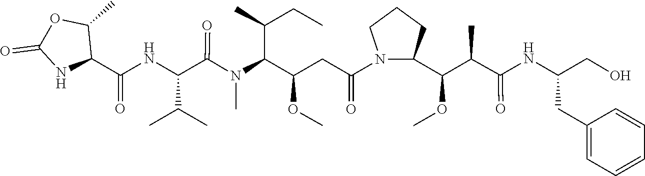 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H]1NC(=O)O[C@@H]1C)C(C)C] |
| 1.49 | 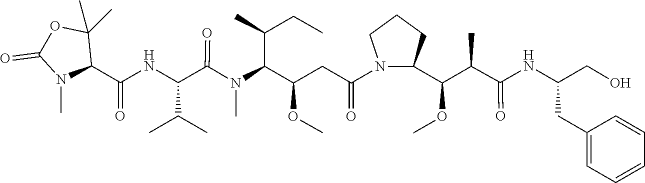 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H]1N(C)C(=O)OC1(C)C)C(C)C] |
| 1.50 | 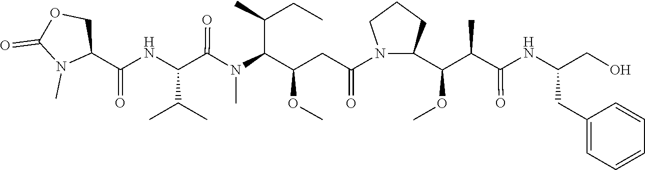 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H]1COC(=O)N1C)C(C)C] |
| 1.51 | 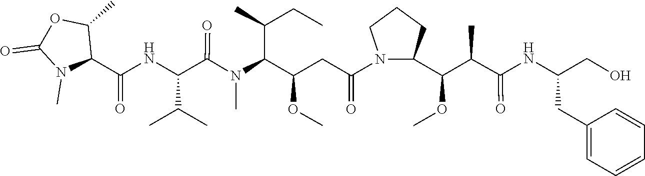 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H]1[C@@H](C)OC(=O)N1C)C(C)C] |
| 1.52 | 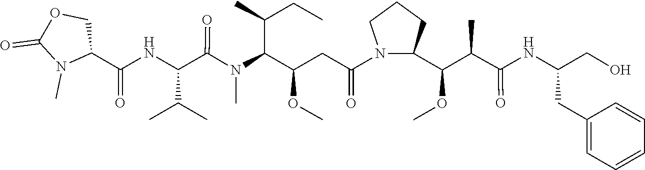 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H]1COC(=O)N1C)C(C)C] |
| 1.53 | 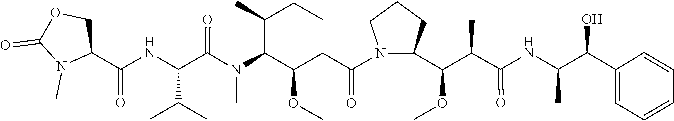 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H]1COC(=O)N1C)C(C)C] |
| 1.54 | 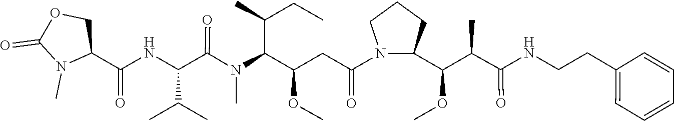 [SMILES: CC[C@H](C)[C@@H]([C@@H](CC(=O)N1CCC[C@H]1[C@H](OC)[C@@H](C)C(=O)NCCc1ccccc1)OC)N(C)C(=O)[C@@H](NC(=O)[C@@H]1COC(=O)N1C)C(C)C] |
| 1.55 | 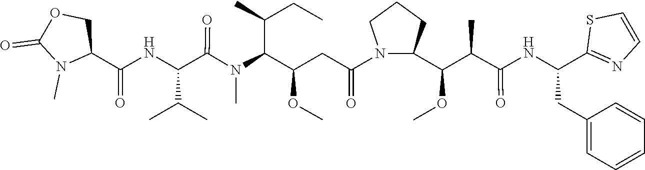 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1COC(=O)N1C)C(C)C] |
| 1.56 | 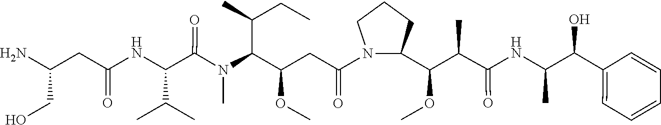 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)C[C@@H](N)CO)C(C)C] |
| 1.57 | 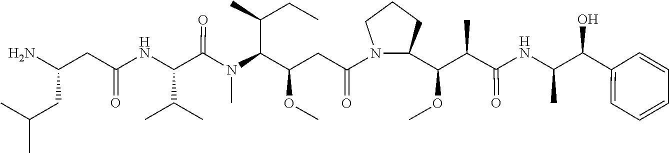 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)C[C@@H](N)CC(C)C)C(C)C] |
| 1.58 | 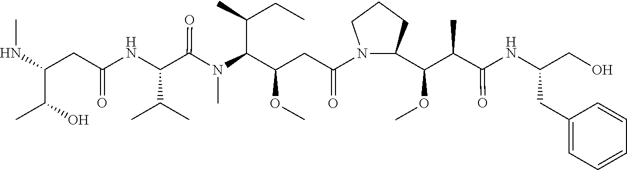 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)C[C@@H](NC)[C@@H](C)O)C(C)C] |
| 1.59 | 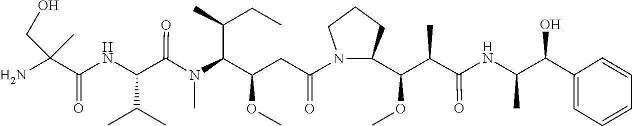 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)C(C)(N)CO)C(C)C] |
| 1.60 | 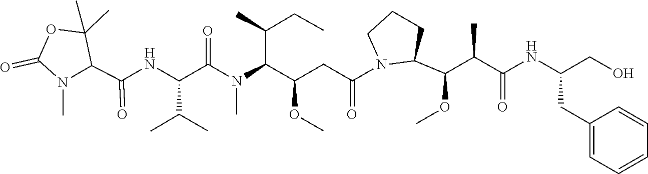 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)C1N(C)C(=O)OC1(C)C)C(C)C] |
| 1.61 | 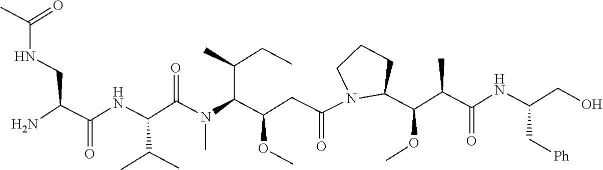 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](N)CNC(C)=O)C(C)C] |
| 1.62 | 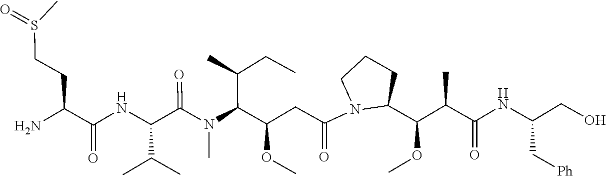 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](N)CCS(C)=O)C(C)C] |
| 1.63 | 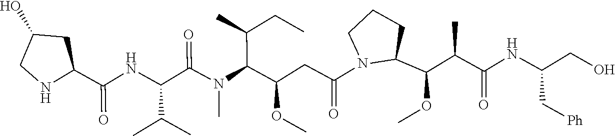 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H]1C[C@@H](O)CN1)C(C)C] |
| 1.64 | 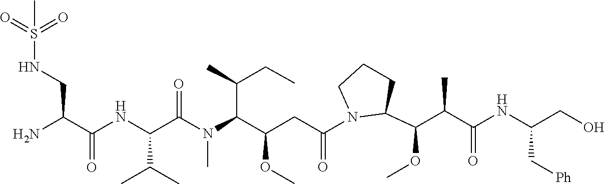 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](N)CNS(C)(=O)=O)C(C)C] |
| 1.65 | 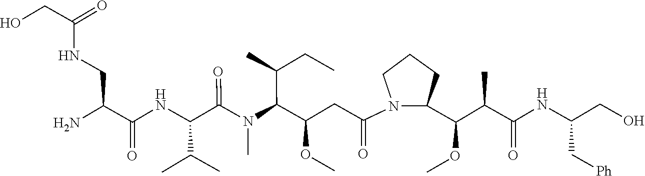 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](N)CNC(=O)CO)C(C)C] |
| 1.66 | 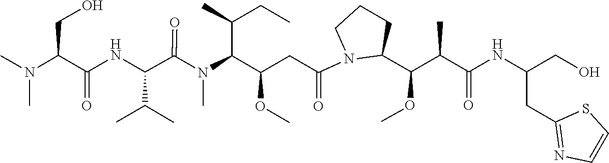 [SMILES: CC[C@H](C)[C@@H]([C@@H](CC(=O)N1CCC[C@H]1[C@H](OC)[C@@H](C)C(=O)NC(CO)Cc1nccs1)OC)N(C)C(=O)[C@@H](NC(=O)[C@H](CO)N(C)C)C(C)C] |
| 1.67 | 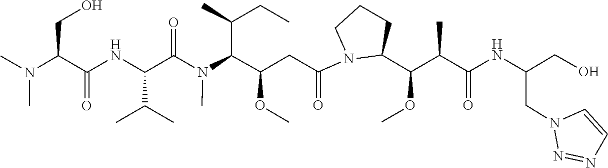 [SMILES: CC[C@H](C)[C@@H]([C@@H](CC(=O)N1CCC[C@H]1[C@H](OC)[C@@H](C)C(=O)NC(CO)Cn1ccnn1)OC)N(C)C(=O)[C@@H](NC(=O)[C@H](CO)N(C)C)C(C)C] |
| 1.68 | 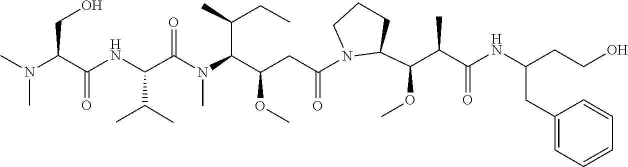 [SMILES: CC[C@H](C)[C@@H]([C@@H](CC(=O)N1CCC[C@H]1[C@H](OC)[C@@H](C)C(=O)NC(CCO)Cc1ccccc1)OC)N(C)C(=O)[C@@H](NC(=O)[C@H](CO)N(C)C)C(C)C] |
| 1.69 | 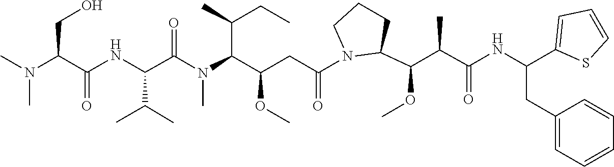 [SMILES: CC[C@H](C)[C@@H]([C@@H](CC(=O)N1CCC[C@H]1[C@H](OC)[C@@H](C)C(=O)NC(Cc1ccccc1)c1cccs1)OC)N(C)C(=O)[C@@H](NC(=O)[C@H](CO)N(C)C)C(C)C] |
| 1.70 | 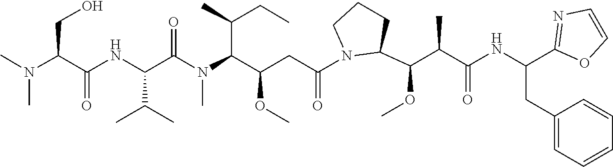 [SMILES: CC[C@H](C)[C@@H]([C@@H](CC(=O)N1CCC[C@H]1[C@H](OC)[C@@H](C)C(=O)NC(Cc1ccccc1)c1ncco1)OC)N(C)C(=O)[C@@H](NC(=O)[C@H](CO)N(C)C)C(C)C] |
| 1.71 | 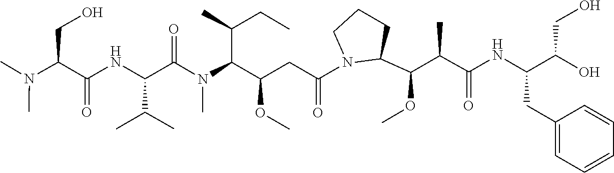 [SMILES: CC[C@H](C)[C@@H]([C@@H](CC(=O)N1CCC[C@H]1[C@H](OC)[C@@H](C)C(=O)N[C@@H](Cc1ccccc1)[C@@H](O)CO)OC)N(C)C(=O)[C@@H](NC(=O)[C@H](CO)N(C)C)C(C)C] |
| 1.72 | 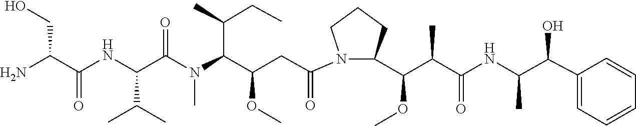 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](N)CO)C(C)C] |

### Drug-Linker Compounds

In some embodiments, when preparing Ligand-Drug Conjugate compounds described herein, it will be desirable to synthesize the full Drug-Linker compound prior to conjugation to a targeting agent, which becomes the Ligand Unit of the Ligand-Drug conjugate compound. In such embodiments, Drug-Linker compounds as described herein, are intermediate compounds. In those embodiments, the Stretcher Unit in a Drug-Linker compound is not yet covalently attached to the Ligand Unit (i.e., is a Stretcher Unit precursor, Z′), and therefore has a functional group for conjugation to a targeting agent. In one embodiment, a Drug-Linker compound comprises an auristatin moiety (shown herein as Formulae (I) and (II), or any subformula thereof) or, and a Linker Unit (Q) through which the Ligand Unit is connected to the Drug Unit.

In another embodiment, a Drug-Linker compound comprises an auristatin compound of Formula (I), or any subformula thereof, as a Drug Unit and a Linker Unit (Q) comprising a Releasable Linker (RL) that is other than a Glycoside (e.g., Glucuronide) Unit through which the Ligand Unit is connected to the conjugated auristatin compound. The Linker Unit comprises, in addition to RL, a Stretcher Unit precursor (Z′) comprising a functional group for conjugation to a targeting agent that is the precursor to the Ligand Unit and thus is capable of (directly or indirectly) connecting the RL to the Ligand Unit. In some of those embodiments a Parallel Connector Unit (B) is present when it is desired to add a Partitioning Agent (S*) as a side chain appendage. In any one of those embodiments, a Connector Unit (A) is present when it is desirable to add more distance between the Stretcher Unit and RL.

In one group of embodiments, a Drug-Linker compound comprises an auristatin compound of Formula (I), or any subformula thereof, and a Linker Unit (Q), wherein Q comprises a Releasable Linker (RL) that is a Glycoside (e.g., Glucuronide) Unit, directly attached to a Stretcher Unit precursor (Z′) or indirectly to Z′ through attachment to intervening component(s) of the Drug-Linker compound's Linker Unit (i.e., A, S* and/or B(S*)), wherein Z′ comprises a functional group capable of forming a covalent bond to a targeting agent.

In another group of embodiments, a Drug-Linker compound comprises an auristatin of Formula (I), or any subformula thereof, and a Linker Unit (Q), wherein Q comprises a Releasable Linker (RL) that is other than a Glycoside (e.g., Glucuronide) Unit (RL), directly attached to a Stretcher Unit precursor (Z′) or indirectly to Z′ through attachment to intervening component(s) of the Drug-Linker compound's Linker Unit (i.e., A, S* and/or B(S*)), wherein Z′ comprises a functional group capable of forming a covalent bond to a targeting agent.

In some embodiments, the Drug-Linker compound has the formula:

Q-D,

or a salt thereof, wherein
Q is a Linker Unit selected from the group consisting of:
   (i) Z′-A-RL-,
   (ii) Z′-A-RL-Y—,
   (iii) Z′-A-S*-RL-,
   (iv) Z′-A-S*-RL-Y—,
   (v) Z′-A-B(S*)—RL-,
   (vi) Z′-A-B(S*)—RL-Y—,
   (vii) Z′-A-,
   (viii) Z′-A-S*—W—,
   (ix) Z′-A-B(S*)—W—,
   (x) Z′-A-S*—W-RL-, and
   (xi) Z′-A-B(S*)—W-RL-;
Z′ is a Stretcher Unit precursor;
A is a bond or a Connector Unit;
B is a Parallel Connector Unit;
S* is a Partitioning Agent;
RL is a Releasable Linker;
W is a Amino Acid Unit;
Y is a Spacer Unit; and
D is a Drug Unit of Formula (I′):

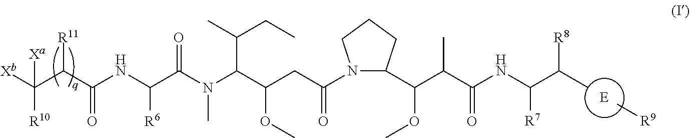

wherein
X^{b} is —NR²—^{#} or —N⁺R¹R⁵—^{#}, wherein the # denotes the point of attachment to Q, and X^{a} is

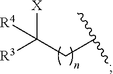

or
X^{a} and X^{b} are taken together with the carbon atom to which they are attached to form

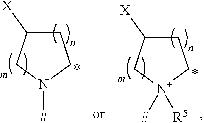

wherein the asterisk denotes the carbon atom of Formula (I) that bears the X^{a} and X^{b} groups, and the # denotes the point of attachment to Q;
R¹ and R⁵ are independently C₁-C₄ alkyl;
X is H, OH, —C(O)NR^{a}R^{b}, —S(O)₂R^{a}, —S(O)—R^{a} —S(O)₂NR^{a}R^{b}, —NHS(O)₂R^{a}, or —NHC(O)R^{a};
R², R³, R⁴, R¹⁰, R^{a}, and R^{b} are each independently H or C₁-C₄ alkyl;
R⁶ is C₁-C₄ alkyl optionally substituted with OH;
R⁷ is H, C₁-C₄ alkyl optionally substituted with one or two OH moieties, or 5-6 membered heteroaryl;
E is phenyl or 5-6 membered heteroaryl;
R⁸, R⁹, and R¹¹ are each independently H or OH;
n is 0, 1, 2, or 3;
m is 1, 2, 3, or 4;
q is 0 or 1; and
wherein when X is H, at least two of R⁷, R⁸, R⁹, and R¹¹ comprise an OH moiety.

In the context of the Drug-Linker Compounds—the assembly is best described in terms of its component groups. While some procedures for the preparation of Drug-Linker compounds are described herein, the order of assembly and the general conditions to prepare the compounds will be well understood by one of skill in the art in view of the teachings of the application.

### Component Groups

1. Drug Unit D

The Drug Units of the Drug-Linker compounds, or Ligand-Drug Conjugate thereof, provided herein are auristatin moieities of the compounds disclosed herein and are referred to herein as Drug Units.

In some embodiments, the Drug Unit D has Formula (I′), as described above.

In some embodiments of Formula (I′), q is 0. In some embodiments, q is 1.

In some embodiments, the Drug Unit D has Formula (Iz′):

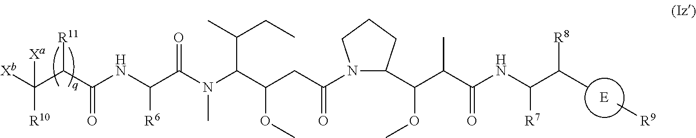

wherein
X^{b} is —NR²—^{#} or —N⁺R¹R⁵—^{#}, wherein the # denotes the point of attachment to Q, and X^{a} is

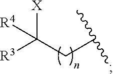

or
X^{a} and X^{b} are taken together with the carbon atom to which they are attached to form

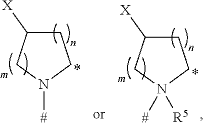

wherein the asterisk denotes the carbon atom of Formula (I) that bears the X^{a} and X^{b} groups, and the # denotes the point of attachment to Q;
R¹ and R⁵ are independently C₁-C₄ alkyl;
X is OH, —C(O)NR^{a}R^{b}, —S(O)₂R^{a}, —S(O)—R^{a} —S(O)₂NR^{a}R^{b}, —NHS(O)₂R^{a}, or —NHC(O)R^{a};
R², R³, R⁴, R¹⁰, R^{a}, and R^{b} are each independently H or C₁-C₄ alkyl;
R⁶ is C₁-C₄ alkyl optionally substituted with OH;
R⁷ is H, C₁-C₄ alkyl optionally substituted with one or two OH moieties, or 5-6 membered heteroaryl;
E is phenyl or 5-6 membered heteroaryl;
R⁸, R⁹, and R¹¹ are each independently H or OH;
n is 0, 1, 2, or 3;
m is 1, 2, 3, or 4; and
q is 0 or 1.

In some embodiments of Formula (I′) or (Iz′), q is 0. In some embodiments, q is 1.

In some embodiments, of Formula (I′) or (Iz′), X^{b} is —NR²—^{#} or —N⁺R¹R⁵—^{#}, wherein the # denotes the point of attachment to Q, and X^{a} is

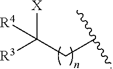

In some embodiments, R¹ and R² are each independently C₁-C₄ alkyl. In some embodiments, R¹ and R⁵ are each independently n-propyl, isopropyl, ethyl, or methyl. In some embodiments, R¹ and R⁵ are each independently ethyl or methyl. In some embodiments, R¹ and R⁵ are methyl. In some embodiments, R³ and R⁴ are each independently C₁-C₄ alkyl. In some embodiments, R³ is H and R⁴ is C₁-C₄ alkyl. In some embodiments, R³ and R⁴ are H. In some embodiments, R³ is H and R⁴ is methyl. In some embodiments, n is 0, 1, 2, or 3. In some embodiments, n is 0, 1, or 2. In some embodiments, n is 0 or 1. In some embodiments, n is 1. In some embodiments, n is 0. In some embodiments, R¹ is methyl, R⁵ is methyl, R³ is H, R⁴ is H, and n is 0. In some embodiments, R² is n-propyl, isopropyl, ethyl or methyl. In some embodiments, R² is ethyl or methyl. In some embodiments, R² is methyl. In some embodiments, R² is H.

In some embodiments of Formula (I′) or (Iz′), X^{a} and X^{b} are taken together with the carbon atom to which they are attached to form

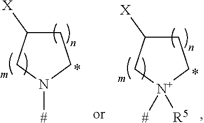

wherein the asterisk denotes the carbon atom of Formula (I) that bears the X^{a} and X^{b} groups, and the # denotes the point of attachment to Q. In some embodiments, R⁵ is n-propyl, isopropyl, ethyl, or methyl. In some embodiments, R⁵ is ethyl or methyl. In some embodiments, R⁵ is ethyl. In some embodiments, R⁵ is methyl. In some embodiments, m is 1, 2, or 3. In some embodiments, m is 1 or 2. In some embodiments, m is 2 or 3. In some embodiments, m is 2. In some embodiments, n is 0, 1, or 2. In some embodiments, n is 0 or 1. In some embodiments, m is 1 or 2 and n is 0 or 1.

In some embodiments of Formula (I′), X is H, OH, —C(O)NR^{a}R^{b}, —S(O)₂R^{a}, —S(O)—R^{a}, —S(O)₂NR^{a}R^{b}, —NHS(O)₂R^{a}, or —NHC(O)R^{a}. In some embodiments, X is OH, —S(O)₂R^{a}, —S(O)—R^{a}, —S(O)₂NR^{a}R^{b}, —NHS(O)₂R^{a}, or —NHC(O)R^{a}. In some embodiments, X is H or OH. In some embodiments, X is OH. In some embodiments, X is OH, —NHS(O)₂R^{a}, or —NHC(O)R^{a}. In some embodiments, X is OH, —S(O)₂R^{a}, —S(O)—R^{a}, or —S(O)₂NR^{a}R^{b}. In some embodiments, X is —NHS(O)₂R^{a}, or —NHC(O)R^{a}. In some embodiments, X is —S(O)₂R^{a}, —S(O)—R^{a}, or —S(O)₂NR^{a}R^{b}. In some embodiments, X is H, OH, —NHS(O)₂R^{a}, or —NHC(O)R^{a}. In some embodiments, X is H, OH, —S(O)₂R^{a}, —S(O)—R^{a}, or —S(O)₂NR^{a}R^{b}. In some embodiments, X is H, —NHS(O)₂R^{a}, or —NHC(O)R^{a}. In some embodiments, X is H, —S(O)₂R^{a}, —S(O)—R^{a}, or —S(O)₂NR^{a}R^{b}. In some embodiments, X is H, OH, or —C(O)NR^{a}R^{b}. In some embodiments, X is OH or —C(O)NR^{a}R^{b}. In some embodiments, X is H or —C(O)NR^{a}R^{b}. In some embodiments, R^{a} and R^{b} are independently H, n-propyl, isopropyl, ethyl, or methyl. In some embodiments, R^{a} and R^{b} are independently H, ethyl, or methyl. In some embodiments, R^{a} and R^{b} are independently H or methyl. In some embodiments, R^{a} is H and R^{b} is n-propyl, isopropyl, ethyl, or methyl. In some embodiments, R^{a} is H and R^{b} is methyl.

In some embodiments of Formula (Iz′), X is OH, —C(O)NR^{a}R^{b}, —S(O)₂R^{a}, —S(O)—R^{a}, —S(O)₂NR^{a}R^{b}, —NHS(O)₂R^{a}, or —NHC(O)R^{a}. In some embodiments, X is OH. In some embodiments, X is OH, —NHS(O)₂R^{a}, or —NHC(O)R^{a}. In some embodiments, X is OH, —S(O)₂R^{a}, —S(O)—R^{a}, or —S(O)₂NR^{a}R^{b}. In some embodiments, X is —NHS(O)₂R^{a}, or —NHC(O)R^{a}. In some embodiments, X is —S(O)₂R^{a}, —S(O)—R^{a}, or —S(O)₂NR^{a}R^{b}. In some embodiments, X is OH or —C(O)NR^{a}R^{b}. In some embodiments, X is —C(O)NR^{a}R^{b}. In some embodiments, R^{a} and R^{b} are independently H, n-propyl, isopropyl, ethyl, or methyl. In some embodiments, R^{a} and R^{b} are independently H, ethyl, or methyl. In some embodiments, R^{a} and R^{b} are independently H or methyl. In some embodiments, R^{a} is H and R^{b} is n-propyl, isopropyl, ethyl, or methyl. In some embodiments, R^{a} is H and R^{b} is methyl.

In some embodiments of Formula (I′) or (Iz′), X^{b} is —N⁺R¹R⁵—^{#} and X^{a} is

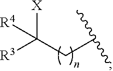

X is OH, R¹ is methyl or methyl, R⁵ is methyl, R³ is H, R⁴ is H, and n is 0. In some embodiments, X is OH, R¹ is methyl, R⁵ is methyl, R³ is H, R⁴ is H, and n is 0. In some embodiments, X is OH, R³ and R⁴ are H, and n is 0.

In some embodiments of Formula (I′) or (Iz′), X^{b} is —NR²—^{#}, X^{a} is

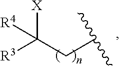

X is OH, and R² is H or C₁-C₄ alkyl. In some embodiments, X^{b} is —NR²—^{#} and X^{a} is

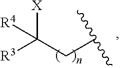

X is OH, and R² is H. In some embodiments, X^{b} is —NR²—^{#} and X^{a} is

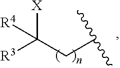

X is OH, and R² is C₁-C₄ alkyl.

In some embodiments or Formula (I′) or (Iz′), R⁶ is n-propyl, isopropyl, ethyl, or methyl. In some embodiments, R⁶ is ethyl or methyl. In some embodiments, R⁶ is isopropyl. In some embodiments, R⁶ is n-propyl, isopropyl, ethyl, or methyl, each of which is substituted with OH. In some embodiments, R⁶ is isopropyl substituted with OH. In some embodiments, R⁶ is ethyl substituted with OH. In some embodiments, R⁶ is methyl substituted with OH.

In some embodiments of Formula (I′) or (Iz′), R⁷ is C₁-C₄ alkyl substituted with OH. In some embodiments, R⁷ is C₁-C₄ alkyl substituted by two OH moieties. In some embodiments, R⁷ is n-propyl, isopropyl, ethyl, or methyl, each of which is substituted with OH. In some embodiments, R⁷ is C₁-C₄ alkyl. In some embodiments, R⁷ is n-propyl, isopropyl, ethyl, or methyl. In some embodiments, R⁷ is methyl. In some embodiments, R⁷ is —CH₂CH₂OH or CH₂OH. In some embodiments, R⁷ is —CH₂OH. In some embodiments, R⁷ is 5-6 membered heteroaryl. In some embodiments, R⁷ is 5-membered heteroaryl. In some embodiments, R⁷ is thiazolyl.

In some embodiments of Formula (I′) or (Iz′), R⁸, R⁹, and R¹¹ are H. In some embodiments, R⁸ is OH, R⁹ is H, and R¹¹ is H. In some embodiments, R⁸ and R⁹ are each OH and R¹¹ is H. In some embodiments, R⁸ is OH, R⁹ is H, and R¹¹ is OH. In some embodiments, R⁸ is H, R⁹ is H, and R¹¹ is OH.

In some embodiments of Formula (I′) or (Iz′), E is an optionally substituted 6-membered ring. In some embodiments, E is pyridine or phenyl, each of which is optionally substituted. In some embodiments, E is optionally substituted phenyl. In some embodiments, E is optionally substituted 5-6 membered heteroaryl. In some embodiments, E is optionally substituted 5-membered heteroaryl. In some embodiments, E is pyridine or phenyl, each of which is unsubstituted. In some embodiments, E is unsubstituted phenyl. In some embodiments, E is unsubstituted 5-6 membered heteroaryl. In some embodiments, E is unsubstituted 5-membered heteroaryl.

In some embodiments of Formula (I′) or (Iz′), R⁷ is —CH₂OH, R⁸ is H, R⁹ is H, and E is phenyl.

In some embodiments, the Drug Unit D has Formula (Ia′) or (Ia″):

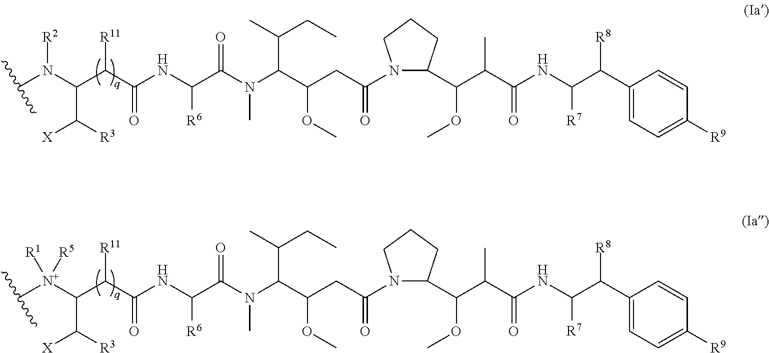

or a salt thereof, wherein
R¹ and R⁵ are independently C₁-C₄ alkyl;
X is H, OH, —C(O)NR^{a}R^{b}, —S(O)₂R^{a}, —S(O)—R^{a} —S(O)₂NR^{a}R^{b}, —NHS(O)₂R^{a}, or —NHC(O)R^{a};
R², R³, R^{a}, and R^{b} are each independently H or C₁-C₄ alkyl;
R⁶ is C₁-C₄ alkyl;
R⁷ is H, C₁-C₄ alkyl optionally substituted with OH, or 5-6 membered heteroaryl;
R⁸, R⁹, and R¹¹ are each independently H or OH;
q is 0 or 1; and
the wavy line is the site of attachment to the rest of the Drug-Linker compound.

In some embodiments of Formula (Ia′) and (Ia″), q is 0. In some embodiments, q is 1.

In some embodiments of Formula (Ia′), R² is C₁-C₄-alkyl. In some embodiments, R² is n-propyl, isopropyl, ethyl, or methyl. R² is ethyl or methyl. R² is methyl. In some embodiments, R² is H. In some embodiments, R² is ethyl, methyl, or H. In some embodiments, R² is methyl or H.

In some embodiments of Formula (Ia″), R¹ and R⁵ are each independently n-propyl, isopropyl, ethyl, or methyl. In some embodiments, R¹ and R⁵ are each independently ethyl or methyl. In some embodiments, R¹ and R⁵ are each methyl. In some embodiments, R¹ is methyl and R⁵ is n-propyl, isopropyl, ethyl, or methyl. In some embodiments, R¹ is methyl and R⁵ is ethyl or methyl. In some embodiments, R¹ and R⁵ are methyl.

In some embodiments of Formula (Ia′) and (Ia″), X is H, OH, —C(O)NR^{a}R^{b}, —S(O)₂R^{a}, —S(O)—R^{a}, —S(O)₂NR^{a}R^{b}, —NHS(O)₂R^{a}, or —NHC(O)R^{a}. In some embodiments, X is OH, —S(O)₂R^{a}, —S(O)—R^{a}, —S(O)₂NR^{a}R^{b}, —NHS(O)₂R^{a}, or —NHC(O)R^{a}. In some embodiments, X is H or OH. In some embodiments, X is OH. In some embodiments, X is OH, —NHS(O)₂R^{a}, or —NHC(O)R^{a}. In some embodiments, X is OH, —S(O)₂R^{a}, —S(O)—R^{a}, or —S(O)₂NR^{a}R^{b}. In some embodiments, X is —NHS(O)₂R^{a}, or —NHC(O)R^{a}. In some embodiments, X is —S(O)₂R^{a}, —S(O)—R^{a}, or —S(O)₂NR^{a}R^{b}. In some embodiments, X is H, OH, —NHS(O)₂R^{a}, or —NHC(O)R^{a}. In some embodiments, X is H, OH, —S(O)₂R^{a}, —S(O)—R^{a}, or —S(O)₂NR^{a}R^{b}. In some embodiments, X is H, —NHS(O)₂R^{a}, or —NHC(O)R^{a}. In some embodiments, X is H, —S(O)₂R^{a}, —S(O)—R^{a}, or —S(O)₂NR^{a}R^{b}. In some embodiments, X is H, OH, or —C(O)NR^{a}R^{b}. In some embodiments, X is OH or —C(O)NR^{a}R^{b}. In some embodiments, X is H or —C(O)NR^{a}R^{b}. In some embodiments, R^{a} and R^{b} are independently H, n-propyl, isopropyl, ethyl, or methyl. In some embodiments, R^{a} and R^{b} are independently H, ethyl, or methyl. In some embodiments, R^{a} and R^{b} are independently H or methyl. In some embodiments, R^{a} is H and R^{b} is n-propyl, isopropyl, ethyl, or methyl. In some embodiments, R^{a} is H and R^{b} is methyl.

In some embodiments of Formula (Ia′) and (Ia″), R³ is H or methyl. In some embodiments, R³ is H. In some embodiments, R³ is methyl.

In some embodiments of Formula (Ia′) and (Ia″), R⁶ is n-propyl, isopropyl, ethyl, or methyl. In some embodiments, R⁶ is ethyl or methyl. In some embodiments, R⁶ is isopropyl. In some embodiments, R⁶ is n-propyl, isopropyl, ethyl, or methyl, each of which is substituted with OH. In some embodiments, R⁶ is isopropyl substituted with OH. In some embodiments, R⁶ is ethyl substituted with OH. In some embodiments, R⁶ is methyl substituted with OH.

In some embodiments of Formula (Ia′) and (Ia″), R⁷ is C₁-C₄ alkyl substituted with OH. In some embodiments, R⁷ is n-propyl, isopropyl, ethyl, or methyl, each of which is substituted with OH. In some embodiments, R⁷ is —CH₂CH₂OH or CH₂OH. In some embodiments, R⁷ is —CH₂OH. In some embodiments, R⁷ is C₁-C₄ alkyl. In some embodiments, R⁷ is n-propyl, isopropyl, ethyl, or methyl. In some embodiments, R⁷ is methyl. In some embodiments, R⁷ is 5-6 membered heteroaryl. In some embodiments, R⁷ is 5-membered heteroaryl. In some embodiments, R⁷ is thiazolyl.

In some embodiments of Formula (Ia′) and (Ia″), R⁸, R⁹, and R¹¹ are H. In some embodiments, R⁸ is OH, R⁹ is H, and R¹¹ is H. In some embodiments, R⁸ and R⁹ are each OH and R¹¹ is H. In some embodiments, R⁸ is OH, R⁹ is H, and R¹¹ is OH. In some embodiments, R⁸ is H, R⁹ is H, and R¹¹ is OH.

In some embodiments of Formula (Ia′), X is OH, R³ is H or methyl, R² is C₁-C₄ alkyl, R₆ is C₁-C₄ alkyl, R⁷ is C₁-C₄ alkyl optionally substituted with OH, R⁸ is H or C₁-C₄ alkyl, and R⁹ is H. In some embodiments, q is 0, X is OH, R³ is H or methyl, R² is C₁-C₄ alkyl, R₆ is C₁-C₄ alkyl, R⁷ is C₁-C₄ alkyl optionally substituted with OH, R⁸ is H or C₁-C₄ alkyl, and R⁹ is H.

In some embodiments of Formula (Ia″), X is OH, R³ is H or methyl, R¹ C₁-C₄ alkyl, R⁵ is C₁-C₄ alkyl, R₆ is C₁-C₄ alkyl, R⁷ is C₁-C₄ alkyl optionally substituted with OH, R⁸ is H or C₁-C₄ alkyl, and R⁹ is H. In some embodiments, q is 0, X is OH, R³ is H or methyl, R¹ C₁-C₄ alkyl, R⁵ is C₁-C₄ alkyl, R₆ is C₁-C₄ alkyl, R⁷ is C₁-C₄ alkyl optionally substituted with OH, R⁸ is H or C₁-C₄ alkyl, and R⁹ is H.

In some embodiments, of the Drug Unit D has Formula (Ib′) or (Ib″):

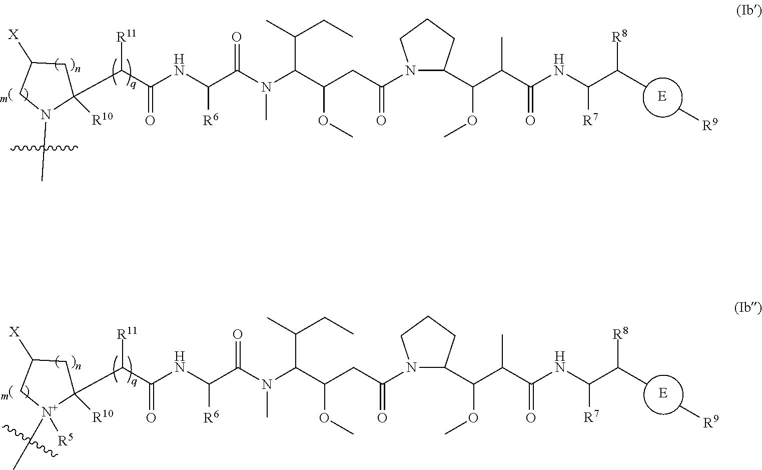

wherein the variables are as defined for Formula (Ib) and the wavy line is the site of attachment to the rest of the Drug-Linker compound.

In some embodiments of Formula (Ib′) and (Ib″), R⁷ is —CH₂OH, R⁸ is H, and R⁹ is H.

In some embodiments, the Drug Unit D has Formula (Ic′) or (Ic″):

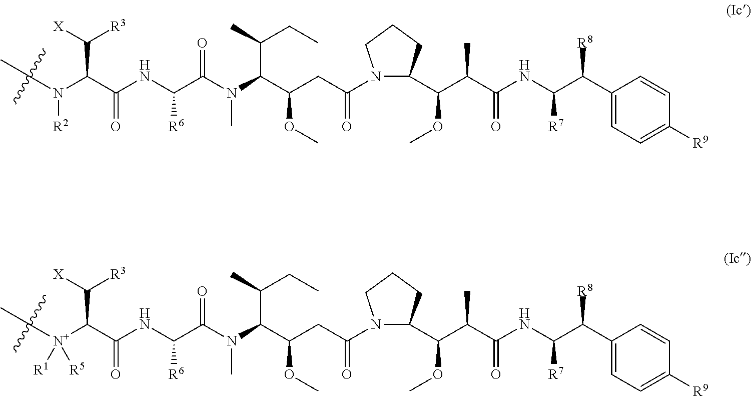

wherein the variables are as defined for Formula (Ic) and the wavy line is the site of attachment to the rest of the Drug-Linker compound.

In some embodiments of Formula (Ic′) or (Ic″), X is OH and R³ is H. In some embodiments, R⁷ is —CH₂OH, R⁸ is H, and R⁹ is H. In some embodiments, X is OH, R⁷ is —CH₂OH, R⁸ is H, and R⁹ is H. In some embodiments, X is OH, R³ is H, R⁷ is —CH₂OH, R⁸ is H, and R⁹ is H.

In some embodiments, the Drug Unit D has the Formula (Id′) or (Id″):

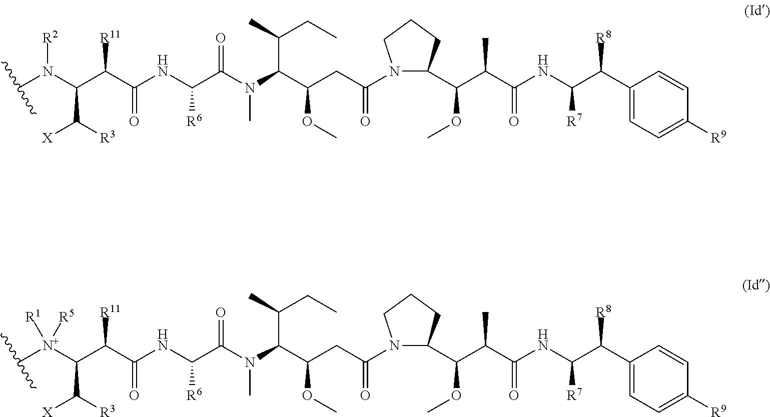

wherein the variables are as defined for Formula (Id) and the wavy line is the site of attachment to the rest of the Drug-Linker compound.

In some embodiments, the Drug Unit D has Formula (Ie′) or (Ie″):

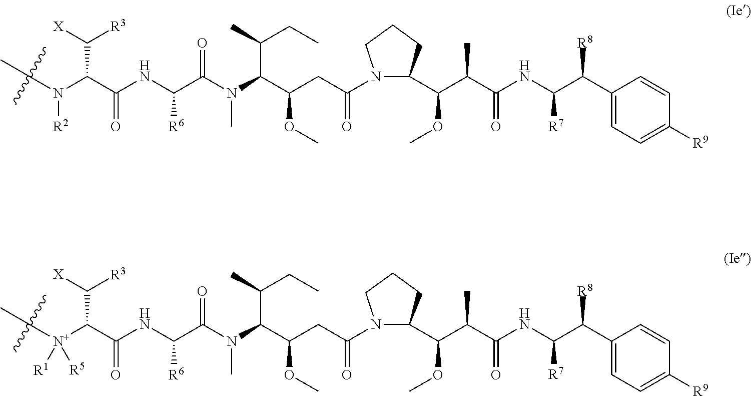

wherein the variables are as defined for Formula (Ie) and the wavy line is the site of attachment to the rest of the Drug-Linker compound.

In some embodiments, the Drug Unit D has the Formula (Id′) or (Id″):

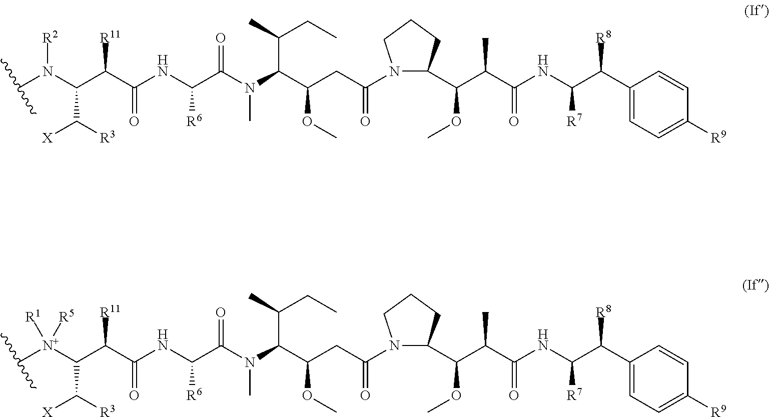

wherein the variables are as defined for Formula (If) and the wavy line is the site of attachment to the rest of the Drug-Linker compound.

In some embodiments of Formula (Id′), (Id″), (If′), or (If″), X is OH and R³ is H. In some embodiments, R⁷ is —CH₂OH, R⁸ is H, and R⁹ is H. In some embodiments, X is OH, R⁷ is —CH₂OH, R⁸ is H, and R⁹ is H. In some embodiments, X is OH, R³ is H, R⁷ is —CH₂OH, R⁸ is H, and R⁹ is H.

In some embodiments of Formula (Ia″), (Ic″), (Id″), (Ie″), or (If″) the quaternized nitrogen atom prevents cyclization of the Drug Unit. In some embodiments, the quaternized nitrogen atom prevents premature release of the Drug Unit from the Drug-Linker compound or moiety (e.g., in a Ligand-Drug Conjugate compound). In some embodiments, the quaternized nitrogen atom prevents cyclization and premature release of the Drug Unit from the Drug-Linker compound or moiety (e.g., in a Ligand-Drug Conjugate compound).

2. Linker Unit Q

As noted above, is some embodiments, the Linker Unit Q has a formula selected from the group consisting of:
(i) Z′-A-RL-,
(ii) Z′-A-RL-Y—,
(iii) Z′-A-S*-RL-,
(iv) Z′-A-S*-RL-Y—,
(v) Z′-A-B(S*)—RL-,
(vi) Z′-A-B(S*)—RL-Y—,
(vii) Z′-A-,
(viii) Z′-A-S*—W—,
(ix) Z′-A-B(S*)—W—,
(x) Z′-A-S*—W-RL-, and
(xi) Z′-A-B(S*)—W-RL-;
   wherein Z′ is a Stretcher Unit; A is a bond or a Connector Unit; B is a Parallel Connector Unit; S* is a Partitioning Agent; RL is Releasable Linker; W is an Amino Acid Unit; and Y is a Spacer Unit.

In other embodiments, the Linker Unit Q has a formula selected from the group consisting of:
(i) Z′-A-RL-,
(ii) Z′-A-RL-Y—,
(iii) Z′-A-S*-RL-,
(iv) Z′-A-S*-RL-Y—,
(x) Z′-A-S*—W-RL-, and
(xi) Z′-A-B(S*)—W-RL-.

In some embodiments, the Linker Unit Q has a formula selected from the group consisting of:
(v) Z′-A-B(S*)—RL-,
(vi) Z′-A-B(S*)—RL-Y—,
(ix) Z′-A-B(S*)—W—, and
(xi) Z′-A-B(S*)—W-RL-.

In some embodiments, the Linker Unit Q has a formula selected from the group consisting of:
(iii) Z′-A-S*-RL-,
(iv) Z′-A-S*-RL-Y—,
(v) Z′-A-B(S*)—RL-,
(vi) Z′-A-B(S*)—RL-Y—,
(viii) Z′-A-S*—W—,
(ix) Z′-A-B(S*)—W—,
(x) Z′-A-S*—W-RL-, and
(xi) Z′-A-B(S*)—W-RL-.

In some embodiments, the Linker Unit Q has a formula selected from the group consisting of:
(viii) Z′-A-S*—W—,
(ix) Z′-A-B(S*)—W—,
(x) Z′-A-S*—W-RL-, and
(xi) Z′-A-B(S*)—W-RL-.

3. Stretcher Unit Z′

A Stretcher Unit (Z) is a component of a Ligand-Drug Conjugate that acts to connect the Ligand Unit to the remainder of the conjugate. A Stretcher Unit precursor (Z′) is a component of a Drug-Linker compound or intermediate thereof has a functional group that can form a bond with a functional group of a targeting ligand to form a Stretcher Unit (Z).

In some embodiments, a Stretcher Unit precursor (Z′) has an electrophilic group that is capable of interacting with a reactive nucleophilic group present on a Ligand Unit (e.g., an antibody) to provide a covalent bond between a Ligand Unit and the Stretcher Unit of a Linker Unit. Nucleophilic groups on an antibody having that capability include but are not limited to, sulfhydryl, hydroxyl and amino functional groups. In some aspects, the heteroatom of the nucleophilic group of an antibody is reactive to an electrophilic group on a Stretcher Unit precursor and can provide a covalent bond between the Ligand Unit and Stretcher Unit of a Linker Unit or Drug-Linker moiety. Useful electrophilic groups for that purpose include, but are not limited to, maleimide, haloacetamide groups, and NHS esters. The electrophilic group provides a convenient site for antibody attachment to form a Ligand-Drug Conjugate compound or Ligand Unit-Linker intermediate compound.

In other embodiments, a Stretcher Unit precursor has a reactive site which has a nucleophilic group that is reactive to an electrophilic group present on a Ligand Unit (e.g., an antibody). Useful electrophilic groups on an antibody for that purpose include, but are not limited to, aldehyde and ketone carbonyl groups. The heteroatom of a nucleophilic group of a Stretcher Unit precursor can react with an electrophilic group on an antibody and form a covalent bond to the antibody. Useful nucleophilic groups on a Stretcher Unit precursor for that purpose include, but are not limited to, hydrazide, hydroxylamine, amino, hydrazine, thiosemicarbazone, hydrazine carboxylate, and arylhydrazide. The electrophilic group on an antibody provides a convenient site for antibody attachment to form a Ligand-Drug Conjugate compound or Ligand Unit-Linker intermediate compound.

In some embodiments, a sulfur atom of a Ligand Unit is bound to a succinimide ring system of a Stretcher Unit formed by reaction of a thiol functional group of a targeting ligand with a maleimide moiety of the corresponding Stretcher Unit precursor. In other embodiments, a thiol functional group of a Ligand Unit reacts with an alpha haloacetamide moiety to provide a sulfur-bonded Stretcher Unit by nucleophilic displacement of its halogen substituent.

Illustrative Stretcher Units prior to conjugation to the Ligand Unit (i.e., Stretcher Unit precursors) comprise a maleimide moiety and are represented by structures including that of formula Z′a

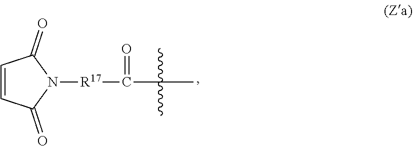

wherein the wavy line adjacent the carbonyl carbon atom indicates attachment to B, A, or S*, in the formulae above, depending on the presence or absence of A and/or B, R¹⁷ is —(CH₂)₁₋₅— or —CH₂CH₂(OCH₂CH₂)₁₋₃₆—. In some embodiments, R¹⁷ is —CH₂—, —(CH₂)₂—, —(CH₂)₃—, —(CH₂)₄—, —(CH₂)₅—, —CH₂CH₂OCH₂CH₂—, —CH₂CH₂(OCH₂CH₂)₂—, —CH₂CH₂(OCH₂CH₂)₃—, —CH₂CH₂(OCH₂CH₂)₄—, —CH₂CH₂(OCH₂CH₂)₅—, —CH₂CH₂(OCH₂CH₂)₆—, —CH₂CH₂(OCH₂CH₂)₇—, —CH₂CH₂(OCH₂CH₂)₈—, —CH₂CH₂(OCH₂CH₂)₁₀—, —CH₂CH₂(OCH₂CH₂)₁₂—, —CH₂CH₂(OCH₂CH₂)₁₄—, —CH₂CH₂(OCH₂CH₂)₁₆—, —CH₂CH₂(OCH₂CH₂)₁₈—, —CH₂CH₂(OCH₂CH₂)₂₀—, —CH₂CH₂(OCH₂CH₂)₂₄—, —CH₂CH₂(OCH₂CH₂)₂₈—, —CH₂CH₂(OCH₂CH₂)₃₂—, or —CH₂CH₂(OCH₂CH₂)₃₆—.

Other illustrative Stretcher Units prior to conjugation to the Ligand Unit (i.e., Stretcher Unit precursors) comprises a maleimide moiety and are represented by structures including that of formula Z′a-BU

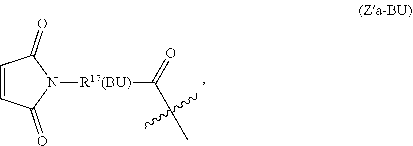

wherein the wavy line adjacent the carbonyl carbon atom indicates attachment to B, A, or S* in the formulae above, depending on the presence or absence of A and/or B, R¹⁷ is R¹⁷ is —(CH₂)₁₋₅— or —CH₂CH₂(OCH₂CH₂)₁₋₅—, substituted with a Basic Unit (BU), such as an optionally substituted aminoalkyl, e.g., —(CH₂)ₓNH₂, —(CH₂)ₓNHR^{a}, and —(CH₂)ₓN(R^{a})₂, wherein subscript x is an integer of from 1-4, preferably R¹⁷ is —CH₂— or —CH₂CH₂— and subscript x is 1 or 2, and each R^{a} is independently selected from the group consisting of C₁₋₆ alkyl and C₁₋₆ haloalkyl, or two R^{a} groups are combined with the nitrogen to which they are attached to form an azetidinyl, pyrrolidinyl or piperidinyl group.

In some embodiments of formula Z′a, a Stretcher Unit precursor (Z′) is represented by one of the following structures:

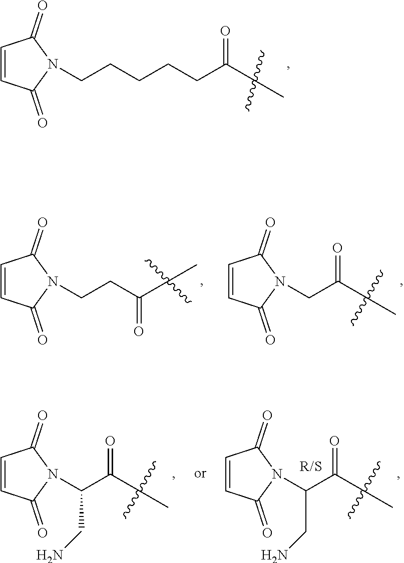 [SMILES: *C(=O)CCCCCN1C(=O)C=CC1=O ; *C(=O)CCN1C(=O)C=CC1=O ; *C(=O)CN1C(=O)C=CC1=O ; *C(=O)[C@H](CN)N1C(=O)C=CC1=O ; *C(=O)C(CN)N1C(=O)C=CC1=O]

wherein the wavy line adjacent to the carbonyl is as defined for Z′a or Z′a-BU.

In other embodiments the Stretcher unit precursor (Z′) comprises a maleimide moiety and is represented by the structure of:

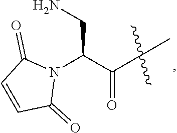 [SMILES: *C(=O)[C@H](CN)N1C(=O)C=CC1=O]

wherein the wavy line adjacent to the carbonyl is as defined for Z′a and the amino group is optional protonated or protected by an amino protecting group.

In Stretcher Units having a BU moiety, it will be understood that the amino functional group of that moiety is typically protected by an amino protecting group during synthesis, e.g., an acid labile protecting group (e.g., BOC).

Illustrative Stretcher Unit precursors covalently attached to a Connector Unit that comprise the structure of Z′a or Z′a-BU in which —R¹⁷— or —R¹⁷(BU)— is —CH₂—, —CH₂CH₂— or —CH(CH₂NH₂)— have the following structures:

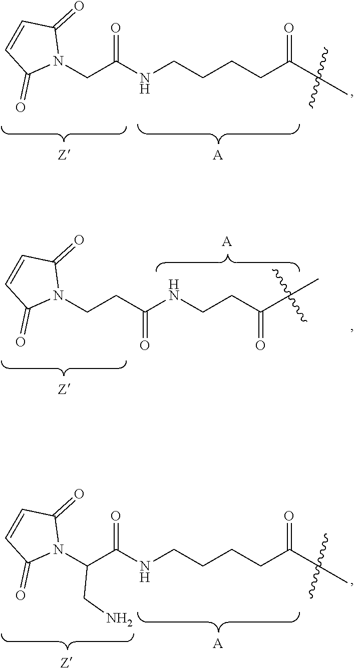

wherein the wavy line adjacent to the carbonyl is as defined for Z′a or Z′a-BU.

Other Stretcher Unit precursors bonded a Connector Unit (A) have the structures above wherein A in any one of the above Z′-A- and Z′(BU)-A- structures is replaced by a Parallel Connector Unit and Partitioning Agent (—B(S*)—) having the structure of

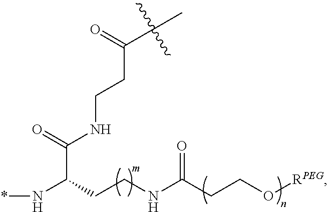

wherein subscript m ranges from 1 to 6; n ranges from 8 to 24; R^{PEG} is a PEG Capping Unit, preferably H, —CH₃, or —CH₂CH₂CO₂H, the asterisk (*) indicates covalent attachment to the Stretcher Unit precursor corresponding in structure to formula Z′a and the wavy line indicates covalent attachment to RL. In instances such as those shown here, the shown PEG group is meant to be exemplary of a variety of Partitioning Agents including PEG groups of different lengths and other Partitioning Agents that are directly attached or modified for attachment to the Parallel Connector Unit.

In some aspects of the prevent invention the Stretcher Unit has a mass of no more than about 1000 daltons, no more than about 500 daltons, no more than about 200 daltons, from about 30, 50 or 100 daltons to about 1000 daltons, from about 30, 50 or 100 daltons to about 500 daltons, or from about 30, 50 or 100 daltons to about 200 daltons.

4. Connector Unit (A)

In some embodiments, a Connector Unit (A), is included in a Drug-Linker Compound in instances where it is desirable to add additional distance between the Stretcher Unit precursor (Z′) and the Releasable Linker. In some embodiments, the extra distance will aid with activation within RL. Accordingly, the Connector Unit (A), when present, extends the framework of the Linker Unit. In that regard, a Connector Unit (A) is covalently bonded with the Stretcher Unit (or its precursor) at one terminus and is covalently bonded to the optional Parallel Connector Unit or the Partitioning Agent (S*) at its other terminus.

The skilled artisan will appreciate that the Connector Unit is any group that serves to provide for attachment of the Releasable Linker to the remainder of the Linker Unit (Q). The Connector Unit can, for example, comprise one or more (e.g., 1-10, preferably, 1, 2, 3, or 4) proteinogenic or non-proteinogenic amino acid, amino alcohol, amino aldehyde, diamino residues. In some embodiments, the Connector Unit is a single proteinogenic or non-proteinogenic amino acid, amino alcohol, amino aldehyde, or diamino residue. An exemplary amino acid capable of acting as Connector units is 0-alanine.

In some of those embodiments, the Connector Unit has the formula denoted below:

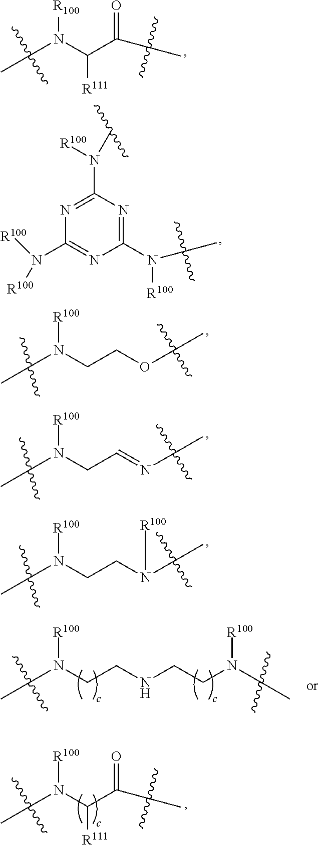

wherein the wavy lines indicate attachment of the Connector Unit within the Drug-Linker Compound; and wherein R¹¹¹ is independently selected from the group consisting of hydrogen, p-hydroxybenzyl, methyl, isopropyl, isobutyl, sec-butyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-,

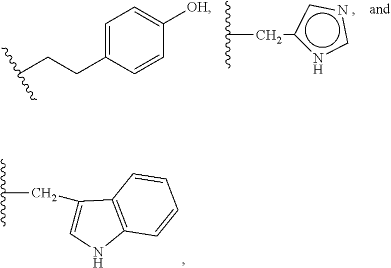

and each R is independently selected from hydrogen or —C₁-C₃ alkyl, preferably hydrogen or CH₃; and subscript c is an independently selected integer from 1 to 10, preferably 1 to 3.

A representative Connector Unit having a carbonyl group for attachment to the Partitioning Agent (S*) or to —B(S*)— is as follows:

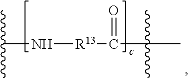

wherein in each instance R¹³ is independently selected from the group consisting of —CH₂CH₂(OCH₂CH₂)ₖ—, —C₁-C₆ alkylene-, —C₃-C₈carbocyclo-, -arylene-, —C₁-C₁₀ heteroalkylene-, —C₃-C₈ heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene-(C₃-C₈ heterocyclo)-, and —(C₃-C₈ heterocyclo)-C₁-C₁₀ alkylene-, wherein subscript k is an integer ranging from 1 to 36 and the subscript c is an integer ranging from 1 to 4. In some embodiments R¹³ is —C₁-C₆ alkylene and c is 1.

Another representative Connector Unit having a carbonyl group for attachment to Partitioning Agent (S*) or to —B(S*)— is as follows:

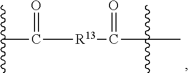

wherein R¹³ is
—CH₂CH₂(OCH₂CH₂)ₖ—, —C₁-C₆ alkylene-, —C₃-C₈carbocyclo-, -arylene-, —C₁-C₁₀ heteroalkylene-, —C₃-C₈ heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene-(C₃-C₈ heterocyclo)-, or —(C₃-C₈ heterocyclo)-C₁-C₁₀ alkylene-, wherein subscript k is an integer ranging from 1 to 36. In some embodiments R¹³ is —C₁-C₆ alkylene.

A representative Connector Unit having a NH moiety that attaches to Partitioning Agent (S*) or to —B(S*)— is as follows:

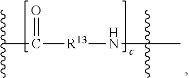

wherein in each instance, R¹³ is independently selected from the group consisting of —CH₂CH₂(OCH₂CH₂)ₖ—, —C₁-C₆ alkylene-, —C₃-C₈carbocyclo-, -arylene-, —C₁-C₁₀ heteroalkylene-, —C₃-C₈ heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene-(C₃-C₈ heterocyclo)-, and —(C₃-C₈ heterocyclo)-C₁-C₁₀ alkylene-, wherein subscript k is an integer ranging from 1 to 36 and subscript c is an integer ranging from 1 to 36. In some embodiments R¹³ is —C₁-C₆ alkylene and subscript c is 1.

Another representative Connector Unit having a NH moiety that attaches to Partitioning Agent (S*) or to —B(S*)— is as follows:

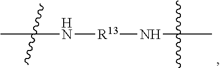

wherein R¹³ is —CH₂CH₂(OCH₂CH₂)ₖ—, —C₁-C₆ alkylene-, —C₃-C₈carbocyclo-, -arylene-, —C₁-C₁₀ heteroalkylene-, —C₃-C₈ heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene-(C₃-C₈ heterocyclo)-, —(C₃-C₈ heterocyclo)-C₁-C₁₀ alkylene-, —C(═O)C₁-C₆ alkylene- or —C₁-C₆ alkylene-C(═O)—C₁-C₆ alkylene, wherein subscript k is an integer ranging from 1 to 36.

Selected embodiments of Connector Units include those having the following structure of:

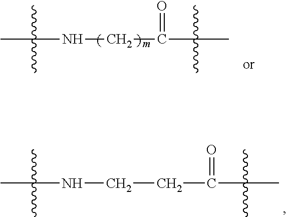

wherein the wavy line adjacent to the nitrogen indicates covalent attachment a Stretcher Unit (Z) (or its precursor Z′), and the wavy line adjacent to the carbonyl indicates covalent attachment to Partitioning Agent (S*) or to —B(S*)—; and m is an integer ranging from 1 to 6, preferably 2 to 6, more preferably 2 to 4.

5. Releasable Linker (RL)

The Releasable Linker (RL) is capable of linking to the Spacer Unit (Y) or the Drug Unit (D). RL comprises a cleavable bond (i.e., a reactive site) that upon action by an enzyme present within a hyper-proliferating cell or hyper-activated immune cells or characteristic of the immediate environment of these abnormal or unwanted cells, or upon non-enzymatic action due to conditions more likely experienced by hyper-proliferating cells in comparison to normal cells, releases free drug. Alternatively, RL comprises a cleavable bond that is more likely acted upon intracellularly in a hyper-proliferating cell or hyper-activated immune cell due to preferential entry into such cells in comparison to normal cells.

### Peptide Releasable Linkers

In some embodiments, the Releasable Linker is a Peptide Releasable Linker. In some embodiments, the Peptide Releasable Linker (RL) will comprise one or more contiguous or non-contiguous sequences of amino acids (e.g., so that RL has 1 to no more than 12 amino acids). The Peptide Releasable Linker can comprise or consist of, for example, an amino acid, a dipeptide, tripeptide, tetrapeptide, pentapeptide, hexapeptide, heptapeptide, octapeptide, nonapeptide, decapeptide, undecapeptide or dodecapeptide unit. In some aspects, in the presence of an enzyme (e.g., a tumor-associated protease), an amide linkage between the amino acids is cleaved, which ultimately leads to release of free drug.

Each amino acid is proteinogenic or non-proteinogenic and/or a D- or L-isomer provided that RL comprises a cleavable bond that, when cleaved, initiates release of the Drug Unit. In some embodiments, the Peptide Releasable Linker will comprise only proteinogenic amino acids. In some aspects, the Peptide Releasable Linker will have from 1 to no more than 12 amino acids in contiguous sequence.

In some embodiments, each amino acid is independently selected from the group consisting of alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, selenocysteine, ornithine, penicillamine, β-alanine, aminoalkanoic acid, aminoalkynoic acid, aminoalkanedioic acid, aminobenzoic acid, amino-heterocyclo-alkanoic acid, heterocyclo-carboxylic acid, citrulline, statine, diaminoalkanoic acid, and derivatives thereof. In some embodiments, each amino acid is independently selected from the group consisting of alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, and selenocysteine. In some embodiments, each amino acid is independently selected from the group consisting of alanine, arginine, aspartic acid, asparagine, histidine, glycine, glutamic acid, glutamine, phenylalanine, lysine, leucine, serine, tyrosine, threonine, isoleucine, proline, tryptophan, and valine. In some embodiments, each amino acid is selected from the proteinogenic or the non-proteinogenic amino acids.

In another embodiment, each amino acid is independently selected from the group consisting of the following L-(proteinogenic) amino acids: alanine, arginine, aspartic acid, asparagine, histidine, glycine, glutamic acid, glutamine, phenylalanine, lysine, leucine, serine, tyrosine, threonine, isoleucine, tryptophan and valine.

In another embodiment, each amino acid is independently selected from the group consisting of the following D-isomers of these proteinogenic amino acids: alanine, arginine, aspartic acid, asparagine, histidine, glycine, glutamic acid, glutamine, phenylalanine, lysine, leucine, serine, tyrosine, threonine, isoleucine, tryptophan and valine.

In certain embodiments, the Peptide Releasable Linker comprises only proteinogenic amino acids. In other embodiments, the Peptide Releasable Linker comprises only non-proteinogenic amino acids. In some embodiments, the Peptide Releasable Linker comprises a proteinogenic amino acid attached to a non-proteinogenic amino acid. In some embodiments, Peptide Releasable Linker comprises a proteinogenic amino acid attached to a D-isomer of a proteinogenic amino acid.

In another embodiment, each amino acid is independently selected from the group consisting of β-alanine, N-methylglycine, glycine, lysine, valine and phenylalanine.

Exemplary Peptide Releasable Linkers include dipeptides or tripeptides with -Val-Lys-Gly-, -Val-Cit-, -Phe-Lys- or -Val-Ala-.

Useful Peptide Releasable Linkers are designed and optimized in their selectivity for enzymatic cleavage by a particular enzyme, for example, a tumor-associated protease. In some embodiments, cleavage of a linkage is catalyzed by cathepsin B, C or D, or a plasmin protease.

In some embodiments, the Peptide Releasable Linker (RL) will be represented by -(-AA-)₁₋₁₂-, or (-AA-AA-)₁₋₆ wherein AA is at each occurrence independently selected from proteinogenic or non-proteinogenic amino acids. In one aspect, AA is at each occurrence independently selected from proteinogenic amino acids. In another aspect, RL is a tripeptide having the formula: AA₁-AA₂-AA₃, wherein AA₁, AA₂ and AA₃ are each independently an amino acid and wherein AA₁ attaches to —NH— and AA₃ attaches to S*. In yet another aspect, AA₃ is gly or β-ala.

In some embodiments, the Peptide Releasable Linker has the formula denoted below in the square brackets, the subscript w is an integer ranging from 1 to 12; or w is 1, 2, 3, 4, 5, 6, 7, 8, 9, 10, 11, or 12; or w is 2, 3, or 4; or w is 3; or w is 4:

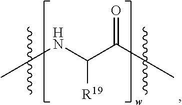

wherein R¹⁹ is, in each instance, independently selected from the group consisting of hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl,

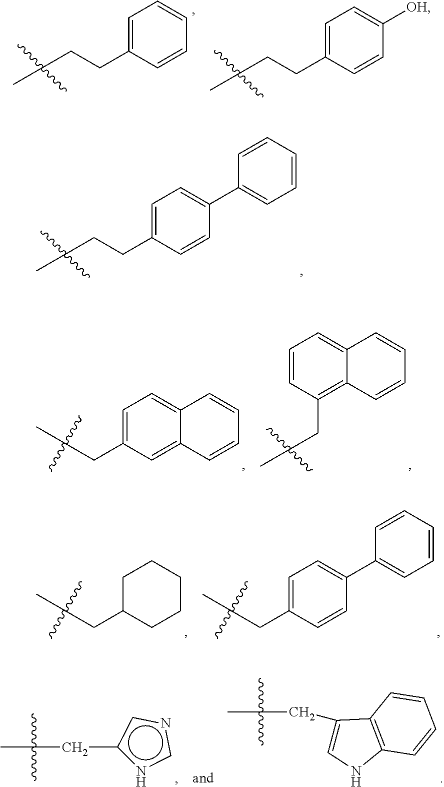

In some aspects, the subscript w is not 3.

In some aspects, each R¹⁹ is independently hydrogen, methyl, isopropyl, isobutyl, sec-butyl, —(CH₂)₃NH₂, or —(CH₂)₄NH₂. In some aspects, each R¹⁹ is independently hydrogen, isopropyl, or —(CH₂)₄NH₂.

Illustrative Peptide Releasable Linkers are represented by formulae (Pa), (Pb) and (Pc):

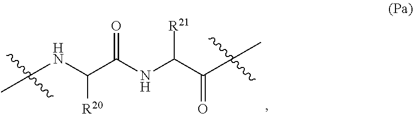

wherein R²⁰ and R²¹ are as follows:

| R²⁰ | R²¹ |
|---|---|
| benzyl | (CH₂)₄NH₂; |
| methyl | (CH₂)₄NH₂; |
| isopropyl | (CH₂)₄NH₂; |
| isopropyl | (CH₂)₃NHCONH₂; |
| benzyl | (CH₂)₃NHCONH₂; |
| isobutyl | (CH₂)₃NHCONH₂; |
| sec-butyl | (CH₂)₃NHCONH₂; |
| 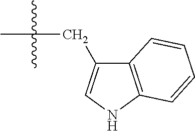 [SMILES: *Cc1c[nH]c2ccccc12] | (CH₂)₃NHCONH₂; |
| benzyl | methyl; and |
| benzyl | (CH₂)₃NHC(═NH)NH₂; |

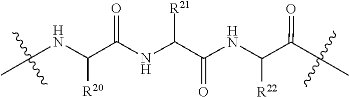

(Pb),

wherein R²⁰, R²¹ and R²² are as follows:

| R²⁰ | R²¹ | R²² |
|---|---|---|
| benzyl | benzyl | —(CH₂)₄NH₂ |
| isopropyl | benzyl | —(CH₂)₄NH₂ |
| H | Benzyl | —(CH₂)₄NH₂ |
| isopropyl | —(CH₂)₄NH₂ | —H |

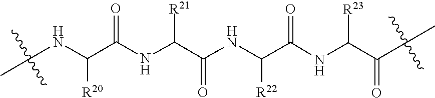

(Pc),

wherein R²⁰, R²¹, R²² and R²³ are as follows:

| R²⁰ | R²¹ | R²² | R²³ |
|---|---|---|---|
| H | benzyl | isobutyl | H; and |
| methyl | isobutyl | methyl | isobutyl. |

In some embodiments, RL comprises a peptide selected from the group consisting of gly-gly, gly-gly-gly, gly-gly-gly-gly, val-gly-gly, val-cit-gly, val-gln-gly, val-glu-gly, phe-lys-gly, leu-lys-gly, gly-val-lys-gly, val-lys-gly-gly, val-lys-gly, val-lys-ala, val-lys-leu, leu-leu-gly, gly-gly-phe-gly, gly-gly-phe-gly-gly, val-gly, and val-lys-β-ala.

In other embodiments, RL comprises a peptide selected from the group consisting of gly-gly-gly, gly-gly-gly-gly, val-gly-gly, val-cit-gly, val-gln-gly, val-glu-gly, phe-lys-gly, leu-lys-gly, gly-val-lys-gly, val-lys-gly-gly, val-lys-gly, val-lys-ala, val-lys-leu, leu-leu-gly, gly-gly-phe-gly, and val-lys-β-ala.

In still other embodiments, RL comprises a peptide selected from the group consisting of gly-gly-gly, val-gly-gly, val-cit-gly, val-gln-gly, val-glu-gly, phe-lys-gly, leu-lys-gly, val-lys-gly, val-lys-ala, val-lys-leu, leu-leu-gly and val-lys-β-ala.

In yet other embodiments, RL comprises a peptide selected from the group consisting of gly-gly-gly-gly, gly-val-lys-gly, val-lys-gly-gly, and gly-gly-phe-gly.

In other embodiments, RL is a peptide selected from the group consisting of val-gln-gly, val-glu-gly, phe-lys-gly, leu-lys-gly, val-lys-gly, val-lys-ala, val-lys-leu, leu-leu-gly and val-lys-β-ala.

In still other embodiments, RL is val-lys-gly.

In still other embodiments, RL is val-lys-β-ala.

In some embodiments, the Releasable Linker RL is

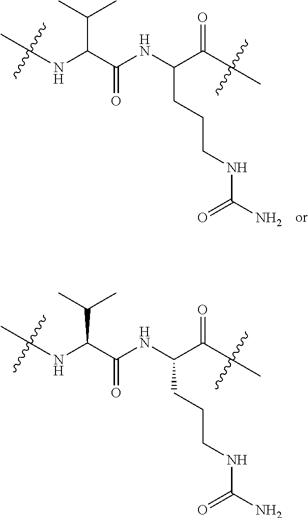 [SMILES: *NC(C(=O)NC(CCCNC(N)=O)C(*)=O)C(C)C ; *N[C@H](C(=O)N[C@@H](CCCNC(N)=O)C(*)=O)C(C)C]

wherein the wavy line adjacent to the —NH— group indicates attachment to the Stretcher Unit Z′ or the Connector Unit A and the wavy line adjacent to the —C(═O)— group indicates attachment to the Spacer Unit Y or the Drug Unit D.

### Glycoside Unit Releasable Linkers

In some embodiments, the Releasable Linker is a Glycoside (e.g., Glucuronide) Unit. In such embodiments, a self-immolation cascade is activated by operation of a glycosidase on a carbohydrate moiety of the Glycoside (e.g., Glucuronide) Unit. A number of sugars are useful in the embodiments described herein. Particular carbohydrate moieties include those of Galactose, Glucose, Mannose, Xylose, Arabinose, Mannose-6-phosphate, Fucose, Rhamnose, Gulose, Allose, 6-deoxy-glucose, Lactose, Maltose, Cellobiose, Gentiobiose, Maltotriose, GlcNAc, GalNAc and maltohexaose.

A Glycoside (e.g., Glucuronide) Unit typically comprises a sugar moiety (Su) linked via an oxygen glycosidic bond to a self-immolative spacer. Cleavage of the oxygen glycosidic bond initiates the self-immolation reaction sequence that result in release of free drug. In some embodiments, the self-immolation sequence is activated from cleavage by 0-glucuronidase of a Glycoside (e.g., Glucuronide) Unit, which is an exemplary glycoside unit. The Glycoside (e.g., Glucuronide) Unit comprises an activation unit and a self-immolative Spacer Unit. The Glycoside (e.g., Glucuronide) Unit comprises a sugar moiety (Su) linked via an oxygen glycosidic bond to a self-immolative Spacer Unit.

In some embodiments, a Glycoside (e.g., Glucuronide) Unit comprises a sugar moiety (Su) linked via an oxygen glycoside bond (—O′—) to a Self-immolative Unit (SP) of the formula:

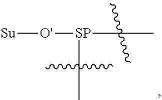

wherein the wavy lines indicate covalent attachment to the Drug Unit or to a Spacer Unit that is attached to the Drug Unit, and to the Stretcher Unit precursor (Z′), either directly or indirectly through the Connector Unit (A) or Parallel Connector Unit (B), Partitioning Agent (S*) or combinations of the Connector Unit and Parallel Connector Unit, as the case may be.

The oxygen glycosidic bond (—O′—) is typically a β-glucuronidase-cleavage site (i.e., Su is from glucuronide), such as a glycoside bond cleavable by human, lysosomal (3-glucuronidase.

In some embodiments, the Glycoside (e.g., Glucuronide) Unit is represented by formula Ga, Gb, or Gc:

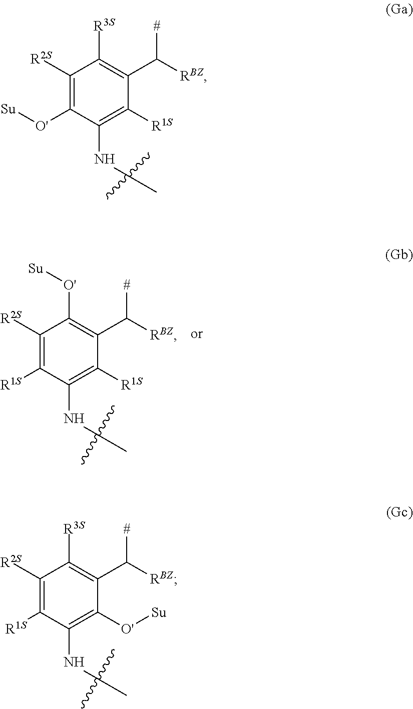

wherein Su is a Sugar moiety, —O′— represents an oxygen glycosidic bond; R^{1S}, R^{2S} and R^{3S} independently are hydrogen, a halogen, —CN, —NO₂, or other electron withdrawing group, or an electron donating group; R^{BZ} is selected from the group consisting of C₁-C₆ alkyl, C₁-C₆ haloalkyl, a PEG unit, a cyclodextrin unit, a polyamide, a hydrophilic peptide, a polysaccharide, and a dendrimer; and wherein the wavy line indicates attachment to a Stretcher Unit precursor (Z′), either directly or indirectly through a Connector Unit or Parallel Connector Unit or Connector unit and Parallel Connector Unit); and # indicates attachment to the Drug Unit or to a Spacer (either directly or indirectly via an intervening functional group or other moiety).

In some embodiments, R^{1S}, R^{2S}, and R^{3S} are independently selected from hydrogen, halogen, —CN, or —NO₂. In some embodiments, R^{1S}, R^{2S} and R^{3S} are each hydrogen. In some embodiments, R^{2S} is an electron withdrawing group, preferably NO₂, and R^{1S} and R^{3S} are each hydrogen.

In some such aspects the activatable self-immolative group capable of glycosidase cleavage to initiate the self-immolative reaction sequence is represented by the formula Gd:

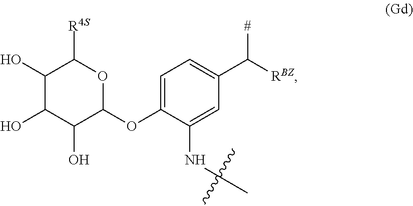

wherein R^{4S} is CH₂OH or —CO₂H, the wavy line indicates covalent attachment to a Stretcher Unit (Z) (or its precursor Z′), either directly or indirectly through a Connector Unit or Parallel Connector Unit or Connector unit and Parallel Connector Unit, and the hash mark (#) indicates covalent attachment to the methylene carbamate unit.

In some embodiments wherein the activatable self-immolative moiety comprises a Glycoside (e.g., Glucuronide) Unit, it is represented by the following formula Ge:

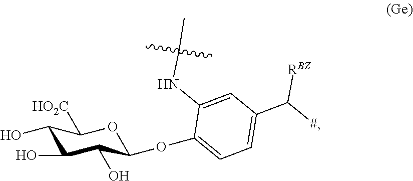

wherein the wavy line indicates covalent attachment to a Stretcher Unit (Z) (or its precursor Z′), either directly or indirectly through a Connector Unit or Parallel Connector Unit or Connector unit and Parallel Connector Unit and the hash mark (#) indicates covalent attachment of the benzylic carbon of a Spacer or functional group attached to the Drug Unit. In some embodiments, the structure of formula Ge is attached to the Drug Unit via a quaternized tertiary amine (N′), wherein the nitrogen atom is from a tertiary amine functional group on the unconjugated Drug Unit.

Another type of Releasable Linker that provides a mechanism for separation of the Drug Unit from the Ligand Unit and other components of the Linker Unit through activation of a self-immolation cascade within the Linker Unit comprises a p-aminobenzyloxycarbonyl (PAB) moiety whose phenylene component is substituted with Jₘ wherein the subscript m indicating the number of substituents is an integer ranging from 0-4, and each J is independently —C₁-C₈ alkyl, —O—(C₁-C₈ alkyl), -halogen, -nitro or -cyano.

In some embodiments, RL is a self-immolative group capable of releasing -D without the need for a separate hydrolysis step or subsequent self-immolative event. In some embodiments, —RL- is a PAB moiety that is linked to the carbonyl of —W— via the amino nitrogen atom of the PAB group, and connected directly to -D via a carbonate group. In related embodiments, —RL- comprises a PAB moiety that is linked to the carbonyl of -A-, —S*— or —B— via the amino nitrogen atom of the PAB group, and connected directly to -D via a carbonate group. Without being bound by any particular theory or mechanism, a possible mechanism of Drug release from RL comprises a PAB moiety in which RL is attached directly to -D via a carbonate group is shown in Toki et al. (2002) J Org. Chem. 67:1866-1872.

In some embodiments, RL units containing a PAB moiety are represented by the formula:

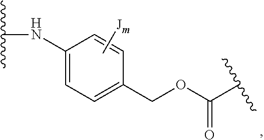

wherein subscript m is an integer ranging from 0-4, and each J is independently —C₁-C₈ alkyl, —O—(C₁-C₈ alkyl), -halogen, -nitro or -cyano.

Other examples of self-immolative groups include, but are not limited to, aromatic compounds that are electronically similar to the PAB moiety such as 2-aminoimidazol-5-methanol derivatives (Hay et al. (1999) Bioorg. Med. Chem. Lett. 9:2237) and ortho or para-aminobenzylacetals. Other RLs undergo cyclization upon amide bond hydrolysis, such as substituted and unsubstituted 4-aminobutyric acid amides (Rodrigues et al., Chemistry Biology, 1995, 2, 223), appropriately substituted bicyclo[2.2.1] and bicyclo[2.2.2] ring systems (Storm, et al., J. Amer. Chem. Soc., 1972, 94, 5815) and 2-aminophenylpropionic acid amides (Amsberry, et al., J. Org. Chem., 1990, 55, 5867).

In one embodiment, RL is a branched bis(hydroxymethyl)styrene (BHMS) unit.

In some embodiments, RL has the formula:

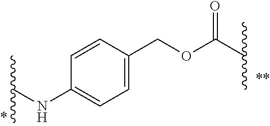 [SMILES: *Nc1ccc(COC(*)=O)cc1]

wherein the wavy line marked with ** indicates the site of attachment to D; and the wavy line marked with * indicates the point of attachment to additional linker components of Q. In some embodiments, a PAB-containing RL is directly attached to the Drug Unit.

6. Partitioning Agent S*

The Ligand-Drug Conjugates described herein can also include a Partitioning Agent (S*). The Partitioning Agent portions are useful, for example, to mask the hydrophobicity of particular Drug Units or Linking Unit components.

Representative Partitioning Agents include polyethylene glycol (PEG) units, cyclodextrin units, polyamides, hydrophilic peptides, polysaccharides and dendrimers.

When the polyethylene glycol (PEG) units, cyclodextrin units, polyamides, hydrophilic peptides, polysaccharides or dendrimers are included in Q, the groups may be present as an ‘in line’ component or as a side chain or branched component. For those embodiments in which a branched version is present, the Linker Units can include a lysine residue (or Parallel Connector Unit, B) that provides simple functional conjugation of, for example, the PEG unit, to the remainder of the Linking Unit.

### Polyethylene Glycol (PEG) Unit

When present, polydisperse PEGs, monodisperse PEGs, and discrete PEGs are used as part of the Partitioning Agents in Compounds of the present invention. Polydisperse PEGs are a heterogeneous mixture of sizes and molecular weights whereas monodisperse PEGs are typically purified from heterogeneous mixtures and are therefore provide a single chain length and molecular weight. Preferred PEG Units are discrete PEGs, compounds that are synthesized in stepwise fashion and not via a polymerization process. Discrete PEGs provide a single molecule with defined and specified chain length.

The PEG Unit provided herein can comprise one or multiple polyethylene glycol chains. A polyethylene glycol chain is composed of at least two ethylene oxide (CH₂CH₂O) subunits. In some embodiments the polyethylene glycol chains are linked together, for example, in a linear, branched or star shaped configuration. Typically, at least one of the PEG chains is derivitized at one end for covalent attachment to an appropriate site on a component of the Linker Unit (e.g. B) or is used as an in-line (e.g., bifunctional) linking group within to covalently join two of the Linker Unit components (e.g., Z-A-S*—RL-, Z-A-S*—RL-Y—). Exemplary attachments within the Linker Unit are by means of non-conditionally cleavable linkages or via conditionally cleavable linkages. Exemplary attachments are via amide linkage, ether linkages, ester linkages, hydrazone linkages, oxime linkages, disulfide linkages, peptide linkages or triazole linkages. In some embodiments, attachment within the Linker Unit is by means of a non-conditionally cleavable linkage. In some embodiments, attachment within the Linker Unit is not via an ester linkage, hydrazone linkage, oxime linkage, or disulfide linkage. In some embodiments, attachment within the Linker Unit is not via a hydrazone linkage.

A conditionally cleavable linkage refers to a linkage that is not substantially sensitive to cleavage while circulating in the plasma but is sensitive to cleavage in an intracellular or intratumoral environment. A non-conditionally cleavable linkage is one that is not substantially sensitive to cleavage in any biological environment. Chemical hydrolysis of a hydrazone, reduction of a disulfide, and enzymatic cleavage of a peptide bond or glycosidic linkage are examples of conditionally cleavable linkages.

In some embodiments, the PEG Unit is directly attached to a Parallel Connector Unit B, wherein the other terminus (or termini) of the PEG Unit is free and untethered and may take the form of a methoxy, carboxylic acid, alcohol or other suitable functional group. The methoxy, carboxylic acid, alcohol or other suitable functional group acts as a cap for the terminal PEG subunit of the PEG Unit. By untethered, it is meant that the PEG Unit will not be attached at that untethered site to a Drug Unit, to an antibody, or to another linking component. The skilled artisan will understand that the PEG Unit in addition to comprising repeating ethylene glycol subunits may also contain non-PEG material (e.g., to facilitate coupling of multiple PEG chains to each other). Non-PEG material refers to the atoms in the PEG Unit that are not part of the repeating —CH₂CH₂O— subunits. In some embodiments provided herein, the PEG Unit comprises two monomeric PEG chains attached to each other via non-PEG elements. In other embodiments provided herein, the PEG Unit comprises two linear PEG chains attached to a central core or Parallel Connector Unit (i.e., the PEG Unit itself is branched).

There are a number of PEG attachment methods available to those skilled in the art, [see, e.g., Goodson, et al. (1990) Bio/Technology 8:343 (PEGylation of interleukin-2 at its glycosylation site after site-directed mutagenesis); EP 0 401 384 (coupling PEG to G-CSF); Malik, et al., (1992) Exp. Hematol. 20:1028-1035 (PEGylation of GM-CSF using tresyl chloride); PCT Pub. No. WO 90/12874 (PEGylation of erythropoietin containing a recombinantly introduced cysteine residue using a cysteine-specific mPEG derivative); U.S. Pat. No. 5,757,078 (PEGylation of EPO peptides); U.S. Pat. No. 5,672,662 (Poly(ethylene glycol) and related polymers monosubstituted with propionic or butanoic acids and functional derivatives thereof for biotechnical applications); U.S. Pat. No. 6,077,939 (PEGylation of an N-terminal .alpha.-carbon of a peptide); Veronese et al., (1985) Appl. Biochem. Biotechnol 11:141-142 (PEGylation of an N-terminal α-carbon of a peptide with PEG-nitrophenylcarbonate (“PEG-NPC”) or PEG-trichlorophenylcarbonate); and Veronese (2001) Biomaterials 22:405-417 (Review article on peptide and protein PEGylation)].

For example, PEG may be covalently bound to amino acid residues via a reactive group. Reactive groups are those to which an activated PEG molecule may be bound (e.g., a free amino or carboxyl group). For example, N-terminal amino acid residues and lysine (K) residues have a free amino group; and C-terminal amino acid residues have a free carboxyl group. Thiol groups (e.g., as found on cysteine residues) can also be useful as a reactive group for attaching PEG. In addition, enzyme-assisted methods for introducing activated groups (e.g., hydrazide, aldehyde, and aromatic-amino groups) specifically at the C-terminus of a polypeptide have been described (see Schwarz, et al. (1990) Methods Enzymol. 184:160; Rose, et al. (1991) Bioconjugate Chem. 2:154; and Gaertner, et al. (1994) J. Biol. Chem. 269:7224].

In some embodiments, PEG molecules may be attached to amino groups using methoxylated PEG (“mPEG”) having different reactive moieties. Non-limiting examples of such reactive moieties include succinimidyl succinate (SS), succinimidyl carbonate (SC), mPEG-imidate, para-nitrophenylcarbonate (NPC), succinimidyl propionate (SPA), and cyanuric chloride. Non-limiting examples of such mPEGs include mPEG-succinimidyl succinate (mPEG-SS), mPEG₂-succinimidyl succinate (mPEG₂-SS); mPEG-succinimidyl carbonate (mPEG-SC), mPEG₂-succinimidyl carbonate (mPEG₂-SC); mPEG-imidate, mPEG-para-nitrophenylcarbonate (mPEG-NPC), mPEG-imidate; mPEG₂-para-nitrophenylcarbonate (mPEG₂-NPC); mPEG-succinimidyl propionate (mPEG-SPA); mPEG₂-succinimidyl propionate (mPEG₂-SPA); mPEG-N-hydroxy-succinimide (mPEG-NHS); mPEG₂-N-hydroxy-succinimide (mPEG₂-NHS); mPEG-cyanuric chloride; mPEG₂-cyanuric chloride; mPEG₂-Lysinol-NPC, and mPEG₂-Lys-NHS.

Generally, at least one of the PEG chains that make up the PEG Unit is functionalized so that it is capable of covalent attachment to other Linker Unit components.

Functionalization includes, for example, via an amine, thiol, NHS ester, maleimide, alkyne, azide, carbonyl, or other functional group. In some embodiments, the PEG Unit further comprises non-PEG material (i.e., material does not comprise —CH₂CH₂O—) that provides coupling to other Linker Unit components or to facilitate coupling of two or more PEG chains.

The presence of the PEG Unit (or other Partitioning Agent) in the Linker Unit can have two potential impacts upon the pharmacokinetics of the resulting Ligand-Drug Conjugate. The desired impact is a decrease in clearance (and consequent increase in exposure) that arises from the reduction in non-specific interactions induced by the exposed hydrophobic elements of the Ligand-Drug Conjugate or to the Drug Unit itself. The second impact is undesired and is a decrease in volume and rate of distribution that sometimes arises from the increase in the molecular weight of the Ligand-Drug Conjugate.

Increasing the number of PEG subunits increases the hydrodynamic radius of a conjugate, typically resulting in decreased diffusivity. In turn, decreased diffusivity typically diminishes the ability of the Ligand-Drug Conjugate to penetrate into a tumor (Schmidt and Wittrup, Mol Cancer Ther 2009; 8:2861-2871). Because of these two competing pharmacokinetic effects, it is desirable to use a PEG that is sufficiently large to decrease the Ligand-Drug Conjugate clearance thus increasing plasma exposure, but not so large as to greatly diminish its diffusivity, to an extent that it interferes with the ability of the Ligand-Drug Conjugate to reach the intended target cell population. See the examples (e.g., examples 1, 18, and 21) of US2016/0310612, which are incorporated by reference herein, for methodology for selecting an optimal PEG size for a particular drug-linker.

In one group of embodiments, the PEG Unit comprises one or more linear PEG chains each having at least 2 subunits, at least 3 subunits, at least 4 subunits, at least 5 subunits, at least 6 subunits, at least 7 subunits, at least 8 subunits, at least 9 subunits, at least 10 subunits, at least 11 subunits, at least 12 subunits, at least 13 subunits, at least 14 subunits, at least 15 subunits, at least 16 subunits, at least 17 subunits, at least 18 subunits, at least 19 subunits, at least 20 subunits, at least 21 subunits, at least 22 subunits, at least 23 subunits, or at least 24 subunits. In some embodiments, the PEG Unit comprises a combined total of at least 4 subunits, at least 6 subunits, at least 8 subunits, at least 10 subunits, or at least 12 subunits. In some such embodiments, the PEG Unit comprises no more than a combined total of about 72 subunits, preferably no more than a combined total of about 36 subunits.

In one group of embodiments, the PEG Unit comprises one or more linear PEG chains each having 2 subunits, 3 subunits, 4 subunits, 5 subunits, 6 subunits, 7 subunits, 8 subunits, 9 subunits, 10 subunits, 11 subunits, 12 subunits, 13 subunits, 14 subunits, 15 subunits, 16 subunits, 17 subunits, 18 subunits, 19 subunits, 20 subunits, 21 subunits, 22 subunits, 23 subunits, or 24 subunits. In some embodiments, the PEG Unit comprises a combined total of 4 subunits, 6 subunits, 8 subunits, 10 subunits, or 12 subunits. In some such embodiments, the PEG Unit comprises no more than a combined total of about 72 subunits, preferably no more than a combined total of about 36 subunits.

In another group of embodiments, the PEG Unit comprises a combined total of from 4 to 72, 4 to 60, 4 to 48, 4 to 36 or 4 to 24 subunits, from 5 to 72, 5 to 60, 5 to 48, 5 to 36 or 5 to 24 subunits, from 6 to 72, 6 to 60, 6 to 48, 6 to 36 or from 6 to 24 subunits, from 7 to 72, 7 to 60, 7 to 48, 7 to 36 or 7 to 24 subunits, from 8 to 72, 8 to 60, 8 to 48, 8 to 36 or 8 to 24 subunits, from 9 to 72, 9 to 60, 9 to 48, 9 to 36 or 9 to 24 subunits, from 10 to 72, 10 to 60, 10 to 48, 10 to 36 or 10 to 24 subunits, from 11 to 72, 11 to 60, 11 to 48, 11 to 36 or 11 to 24 subunits, from 12 to 72, 12 to 60, 12 to 48, 12 to 36 or 12 to 24 subunits, from 13 to 72, 13 to 60, 13 to 48, 13 to 36 or 13 to 24 subunits, from 14 to 72, 14 to 60, 14 to 48, 14 to 36 or 14 to 24 subunits, from 15 to 72, 15 to 60, 15 to 48, 15 to 36 or 15 to 24 subunits, from 16 to 72, 16 to 60, 16 to 48, 16 to 36 or 16 to 24 subunits, from 17 to 72, 17 to 60, 17 to 48, 17 to 36 or 17 to 24 subunits, from 18 to 72, 18 to 60, 18 to 48, 18 to 36 or 18 to 24 subunits, from 19 to 72, 19 to 60, 19 to 48, 19 to 36 or 19 to 24 subunits, from 20 to 72, 20 to 60, 20 to 48, 20 to 36 or 20 to 24 subunits, from 21 to 72, 21 to 60, 21 to 48, 21 to 36 or 21 to 24 subunits, from 22 to 72, 22 to 60, 22 to 48, 22 to 36 or 22 to 24 subunits, from 23 to 72, 23 to 60, 23 to 48, 23 to 36 or 23 to 24 subunits, or from 24 to 72, 24 to 60, 24 to 48, 24 to 36 or 24 subunits.

In some embodiments, the Partitioning Agent S* is a linear PEG Unit comprising from 2 to 20, or from 2 to 12, or from 4 to 12, or 4, 8, or 12 —CH₂CH₂O— subunits. In some embodiments, the linear PEG Unit is connected at one end of the PEG Unit to the RL Unit and at the other end of the PEG Unit to the Stretcher/Connector Units (Z-A-). In some embodiments, the PEG Unit is connected to the RL Unit via a —CH₂CH₂C(O)— group that forms an amide bond with the RL Unit (e.g., —(CH₂CH₂O)ₙ—CH₂CH₂C(O)—RL) and to the Stretcher Unit/Connector Unit (Z-A-) via an —NH— group (e.g., Z-A-NH—(CH₂CH₂O)ₙ—) that forms an amide bond with the Z-A- portion.

Illustrative embodiments for PEG Units that are connected to the RL and Stretcher/Connector Units (Z-A-) are shown below:

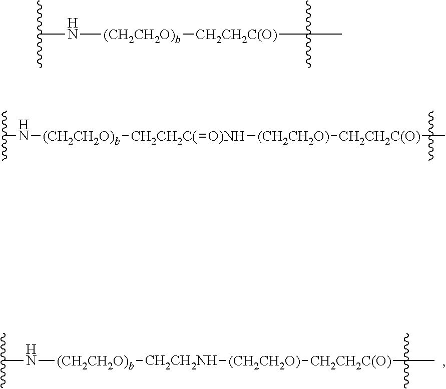

and in a particular embodiment, the PEG Unit is:

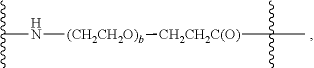

wherein the wavy line on the left indicates the site of attachment to Z-A-, the wavy line on the right indicates the site of attachment to RL, and each b is independently selected from 2 to 72, 4 to 72, 6 to 72, 8 to 72, 10 to 72, 12 to 72, 2 to 24, 4 to 24, 6 to 24, or 8 to 24, 2 to 12, 4 to 12, 6 to 12, and 8 to 12. In some embodiments, subscript b is 2, 4, 8, 12, or 24. In some embodiments, subscript b is 2. In some embodiments, subscript b is 4. In some embodiments, subscript b is 8. In some embodiments, subscript b is 12.

In some embodiments, the linear PEG Unit that is connected to the Parallel Connector Unit at one end and comprises a terminal cap at the other end. In some embodiments, the PEG Unit is connected to the Parallel Connector Unit via a carbonyl group that forms an amide bond with the Parallel Connector Unit lysine residue amino group (e.g., —CH₂CH₂(OCH₂CH₂)ₖ—C(O)—B—, wherein k is an integer from 1 to 36) and includes a PEG Unit terminal cap group selected from the group consisting of C₁₋₄alkyl and C₁₋₄alkyl-CO₂H. In some embodiments, the Partitioning Agent S* is a linear PEG Unit comprising 4, 8, or 12 —CH₂CH₂O— subunits and a terminal methyl cap.

Illustrative linear PEG Units that are used in any of the embodiments provided herein are as follows:

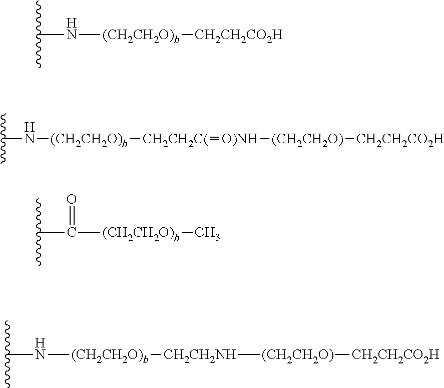

and in a particular embodiment, the PEG Unit is:

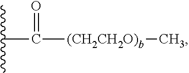

wherein the wavy line indicates site of attachment to the Parallel Connector Unit (B), and each n is independently selected from 4 to 72, 6 to 72, 8 to 72, 10 to 72, 12 to 72, 6 to 24, or 8 to 24. In some embodiments, subscript b is about 4, about 8, about 12, or about 24.

As used to herein, terms “PEG2”, “PEG4”, “PEG8”, and “PEG12” refers to specific embodiments of PEG Unit which comprises the number of PEG subunits (i.e., the number of subscription “b”). For example, “PEG2” refers to embodiments of PEG Unit that comprises 2 PEG subunits, “PEG4” refers to embodiments of PEG Unit that comprises 4 PEG subunits, “PEG8” refers to embodiments of PEG Unit that comprises 8 PEG subunits, and “PEG12” refers to embodiments of PEG Unit that comprises 12 PEG subunits.

As described herein, the PEG unit is selected such that it improves clearance of the resultant Ligand-Drug Conjugate but does not significantly impact the ability of the Conjugate to penetrate into the tumor. In embodiments, the PEG unit to be selected for use will preferably have from 2 subunits to about 24 subunits, from 4 subunits to about 24 subunits, more preferably about 4 subunits to about 12 subunits.

In some embodiments of the present disclosure the PEG Unit is from about 300 daltons to about 5 kilodaltons; from about 300 daltons, to about 4 kilodaltons; from about 300 daltons, to about 3 kilodaltons; from about 300 daltons, to about 2 kilodaltons; or from about 300 daltons, to about 1 kilodalton. In some such aspects, the PEG Unit has at least 6 subunits or at least 8, 10 or 12 subunits. In some such aspects, the PEG Unit has at least 6 subunits or at least 8, 10 or 12 subunits but no more than 72 subunits, preferably no more than 36 subunits.

It will be appreciated that when referring to PEG subunits, and depending on context, the number of subunits can represent an average number, e.g., when referring to a population of Ligand-Drug Conjugates or Drug-Linker Compounds, and/or using polydisperse PEGs.

7. Parallel Connector Unit (B)

In some embodiments, the Ligand-Drug Conjugates and Drug-Linker Compounds will comprise a Parallel Connector Unit to provide a point of attachment to a Partitioning Agent (shown in the Linker Units as —B(S*)—). In some embodiments, the PEG Unit is attached to a Parallel Connector Unit such as lysine as shown below wherein the wavy line and asterisks indicate covalent linkage within the Linker Unit of a Ligand-Drug Conjugate or Drug-Linker Compound:

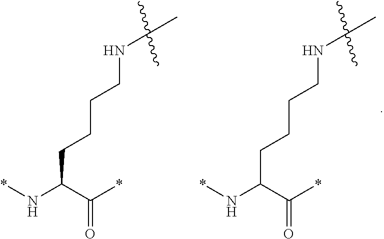 [SMILES: *NCCCC[C@H](N*)C(*)=O ; *NCCCCC(N*)C(*)=O]

In some embodiments, the Parallel Connector Unit (B) and Partitioning Agent (S*) (together, —B(S*)—) have the structure of

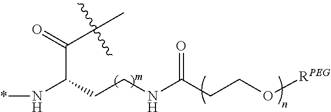

wherein m ranges from 0 to 6; n ranges from 2 to 24; R^{PEG} is a PEG Capping Unit, preferably H, —CH₃, or —CH₂CH₂CO₂H, the asterisk (*) indicates covalent attachment to a Connector Unit A corresponding in formula Za, Za′, Zb′ or Zc′ and the wavy line indicates covalent attachment to the Releasable Linker (RL). In some embodiments, the structure is attached to a Connector Unit A in formula Za or Za′. In some embodiments, n is 2, 4, 8, or 12. In instances such as those shown here, the shown PEG group is meant to be exemplary of a variety of Partitioning Agents including PEG groups of different lengths and other Partitioning Agents that are directly attached or modified for attachment to the Parallel Connector Unit.

8. Spacer Unit (Y)

In some embodiments, the Ligand-Drug Conjugates provided herein will have a Spacer (Y) between the Releasable Linker (RL) and the Drug Unit. The Spacer Unit is a functional group to facilitate attachment of RL to the Drug Unit, or provides additional structural components to further facilitate release of the Drug Unit from the remainder of the Conjugate (e.g., a methylene carbamate unit or a self-immolative para-aminobenzyl (PAB) component).

In those embodiments to further facilitate release of the Drug Unit as free drug, the Spacer Unit Y is represented by one of the following formulae:

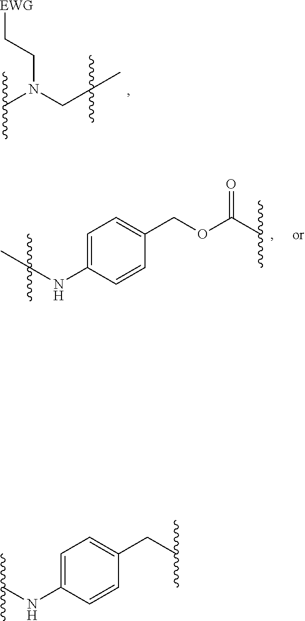

wherein EWG represents an electron-withdrawing group and the wavy lines indicate the site of attachment to the rest of the Drug-Linker compound or salt thereof. In some embodiments, EWG is selected from the group consisting of —CN, —NO₂, —CX₃, —X, —C(═O)OR′, —C(═O)N(R′)₂, —C(═O)R′, —C(═O)X, —S(═O)₂R′, —S(═O)₂OR′, —S(═O)₂NHR′, —S(═O)₂N(R′)₂, —P(═O)(OR′)₂, —P(═O)(CH₃)NHR′, —NO, —N(R′)₃′, wherein X is —F, —Br, —Cl, or —I, and R′ is independently selected from the group consisting of hydrogen and C₁-C₆ alkyl.

In some embodiments, the Spacer Unit-Drug Unit group (—Y-T*-D) is represented by one of the following formulae:

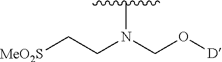

wherein the wavy line adjacent is the point of covalent attachment to RL, T* is as defined above, and D′ represents the remainder of the Drug Unit, wherein T* and D′ together form a Drug Unit of Formula (Ia), or any subformula thereof.

In some embodiments, the Spacer Unit is represented by the formula:

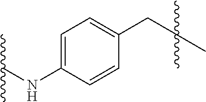 [SMILES: *Cc1ccc(N*)cc1]

wherein the wavy line adjacent to the nitrogen atom is the point of covalent attachment to RL, as defined above, and the wavy line next to the benzylic carbon atom connects to a Drug Unit. In some embodiments, the Drug Unit is attached to the benzylic carbon atom via a quaternized tertiary amine (N+) of D.

In still other embodiments, the Spacer Unit is represented by the formula:

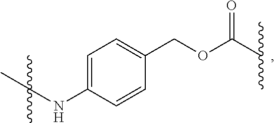 [SMILES: *Nc1ccc(COC(*)=O)cc1]

wherein the wavy line adjacent to the nitrogen atom is the point of covalent attachment to RL, as defined above, and the wavy line next to the —OC(O)— group connects to a Drug Unit. In some embodiments, the Drug Unit is attached via a primary or secondary amine.

In some embodiments, provided herein is a Drug-Linker compound of Table 2, or a salt thereof.

TABLE 2
Drug-Linker compounds

| # | Drug-Linker structure |
|---|---|
| 2.1 | 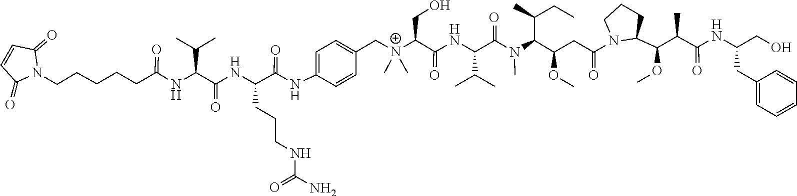 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)[N+](C)(C)Cc1ccc(NC(=O)[C@H](CCCNC(N)=O)NC(=O)[C@@H](NC(=O)CCCCCN2C(=O)C=CC2=O)C(C)C)cc1)C(C)C] |
| 2.2 | 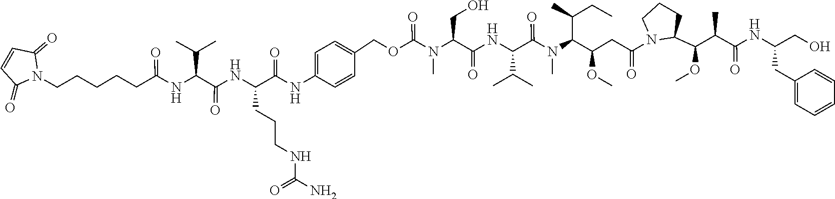 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C] |
| 2.3 | 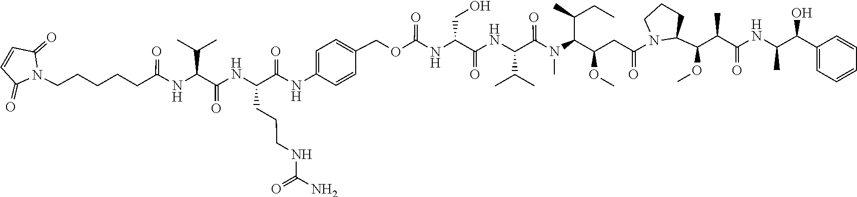 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](CO)NC(=O)OCc1ccc(NC(=O)[C@H](CCCNC(N)=O)NC(=O)[C@@H](NC(=O)CCCCCN2C(=O)C=CC2=O)C(C)C)cc1)C(C)C] |
| 2.4 | 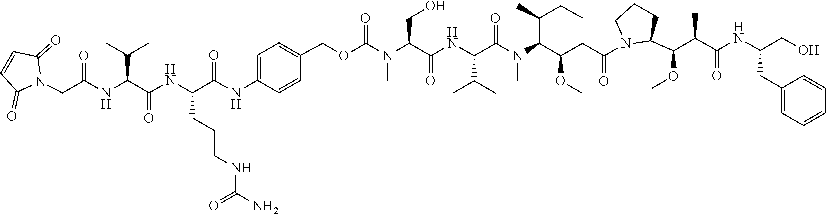 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)N(C)C(=O)OCc1ccc(NC(=O)[C@H](CCCNC(N)=O)NC(=O)[C@@H](NC(=O)CN2C(=O)C=CC2=O)C(C)C)cc1)C(C)C] |
| 2.5 | 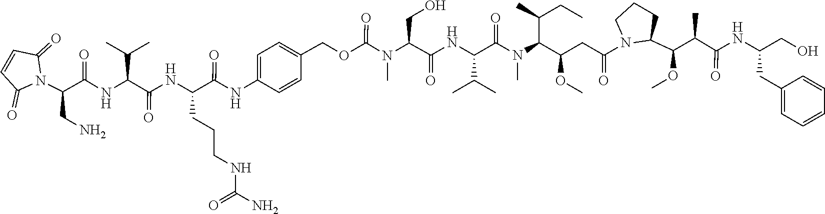 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)N(C)C(=O)OCc1ccc(NC(=O)[C@H](CCCNC(N)=O)NC(=O)[C@@H](NC(=O)[C@@H](CN)N2C(=O)C=CC2=O)C(C)C)cc1)C(C)C] |
| 2.6 | 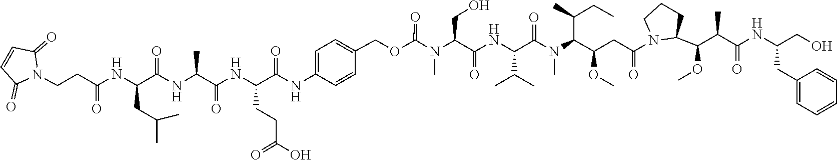 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)N(C)C(=O)OCc1ccc(NC(=O)[C@H](CCC(=O)O)NC(=O)[C@H](C)NC(=O)[C@@H](CC(C)C)NC(=O)CCN2C(=O)C=CC2=O)cc1)C(C)C] |
| 2.7 | 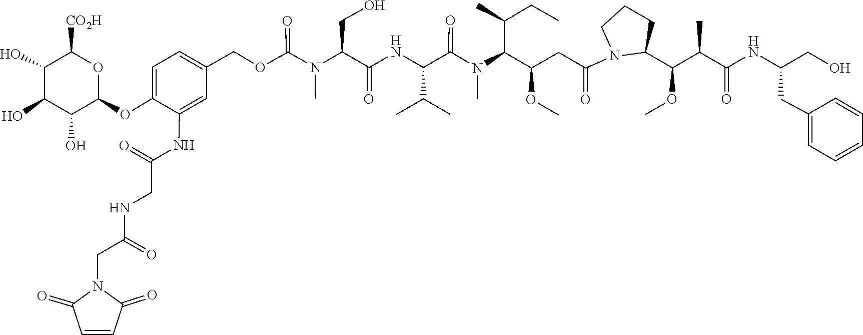 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)N(C)C(=O)OCc1ccc(O[C@@H]2O[C@H](C(=O)O)[C@@H](O)[C@H](O)[C@H]2O)c(NC(=O)CNC(=O)CN2C(=O)C=CC2=O)c1)C(C)C] |
| 2.8 | 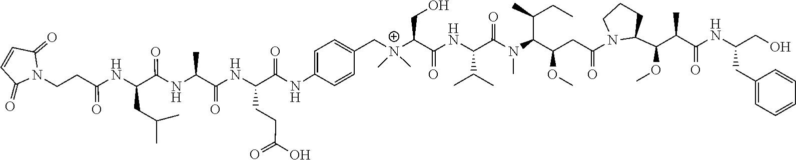 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)[N+](C)(C)Cc1ccc(NC(=O)[C@H](CCC(=O)O)NC(=O)[C@H](C)NC(=O)[C@@H](CC(C)C)NC(=O)CCN2C(=O)C=CC2=O)cc1)C(C)C] |
| 2.9 | 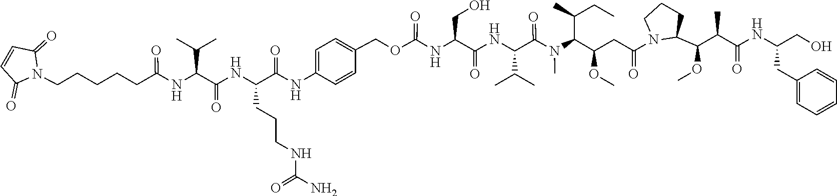 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)NC(=O)OCc1ccc(NC(=O)[C@H](CCCNC(N)=O)NC(=O)[C@@H](NC(=O)CCCCCN2C(=O)C=CC2=O)C(C)C)cc1)C(C)C] |
| 2.10 | 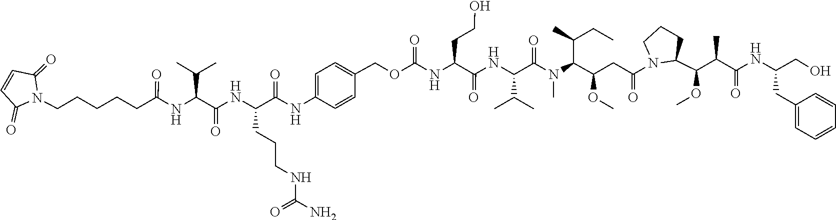 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CCO)NC(=O)OCc1ccc(NC(=O)[C@H](CCCNC(N)=O)NC(=O)[C@@H](NC(=O)CCCCCN2C(=O)C=CC2=O)C(C)C)cc1)C(C)C] |
| 2.11 | 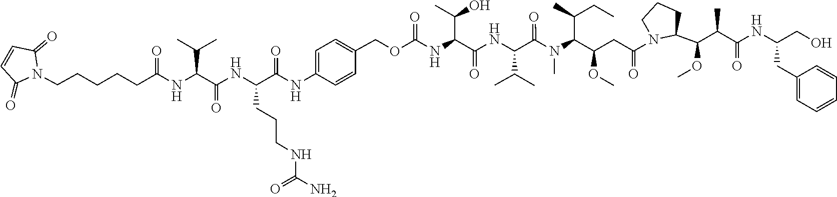 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](NC(=O)OCc1ccc(NC(=O)[C@H](CCCNC(N)=O)NC(=O)[C@@H](NC(=O)CCCCCN2C(=O)C=CC2=O)C(C)C)cc1)[C@@H](C)O)C(C)C] |
| 2.12 | 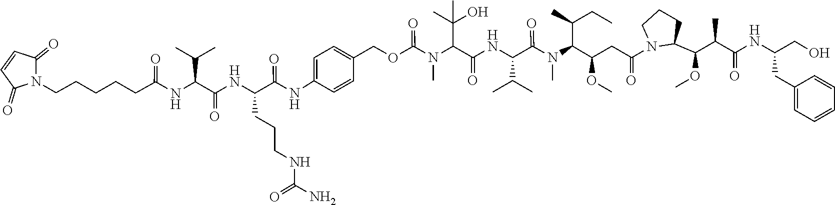 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)C(N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)(C)O)C(C)C] |
| 2.13 | 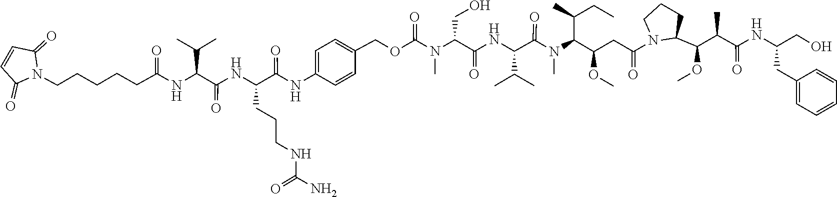 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](CO)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C] |
| 2.14 | 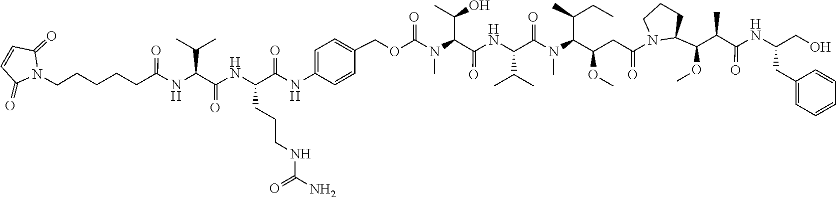 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H]([C@@H](C)O)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C] |
| 2.15 | 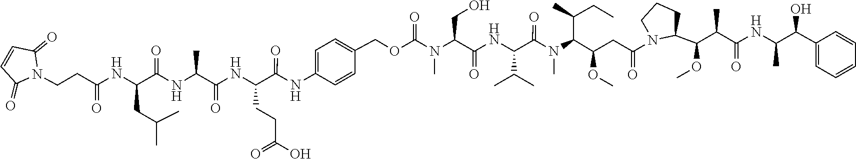 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](CO)N(C)C(=O)OCc1ccc(NC(=O)[C@H](CCC(=O)O)NC(=O)[C@H](C)NC(=O)[C@@H](CC(C)C)NC(=O)CCN2C(=O)C=CC2=O)cc1)C(C)C] |
| 2.16 | 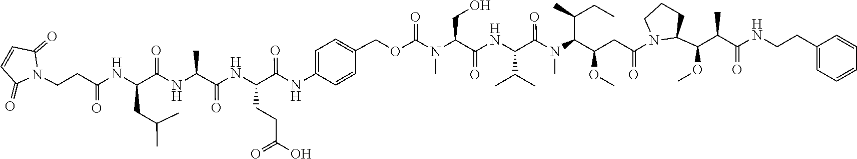 [SMILES: CC[C@H](C)[C@@H]([C@@H](CC(=O)N1CCC[C@H]1[C@H](OC)[C@@H](C)C(=O)NCCc1ccccc1)OC)N(C)C(=O)[C@@H](NC(=O)[C@H](CO)N(C)C(=O)OCc1ccc(NC(=O)[C@H](CCC(=O)O)NC(=O)[C@H](C)NC(=O)[C@@H](CC(C)C)NC(=O)CCN2C(=O)C=CC2=O)cc1)C(C)C] |
| 2.17 | 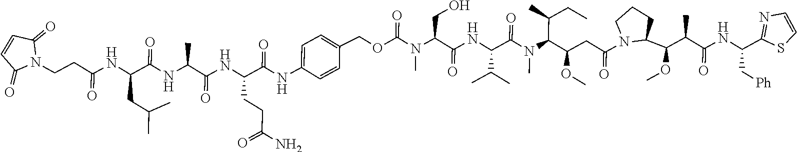 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H](CO)N(C)C(=O)OCc1ccc(NC(=O)[C@H](CCC(N)=O)NC(=O)[C@H](C)NC(=O)[C@@H](CC(C)C)NC(=O)CCN2C(=O)C=CC2=O)cc1)C(C)C] |
| 2.18 | 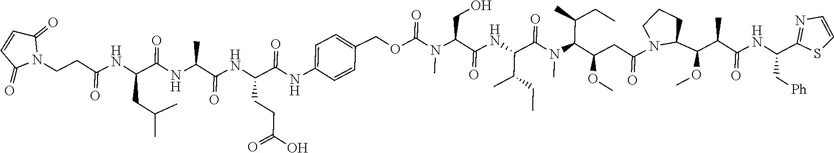 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H](CO)N(C)C(=O)OCc1ccc(NC(=O)[C@H](CCC(=O)O)NC(=O)[C@H](C)NC(=O)[C@@H](CC(C)C)NC(=O)CCN2C(=O)C=CC2=O)cc1)[C@@H](C)CC] |
| 2.19 | 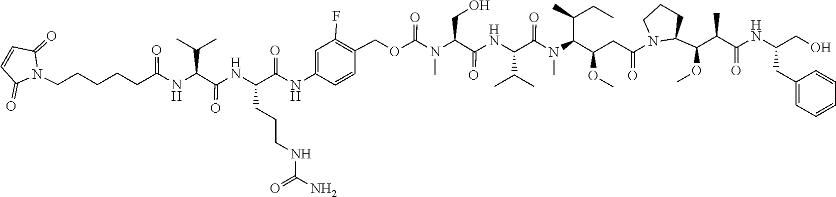 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)N(C)C(=O)OCc1ccc(NC(=O)[C@H](CCCNC(N)=O)NC(=O)[C@@H](NC(=O)CCCCCN2C(=O)C=CC2=O)C(C)C)cc1F)C(C)C] |
| 2.20 | 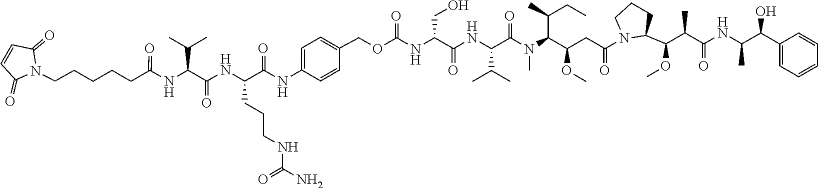 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](CO)NC(=O)OCc1ccc(NC(=O)[C@H](CCCNC(N)=O)NC(=O)[C@@H](NC(=O)CCCCCN2C(=O)C=CC2=O)C(C)C)cc1)C(C)C] |
| 2.21 | 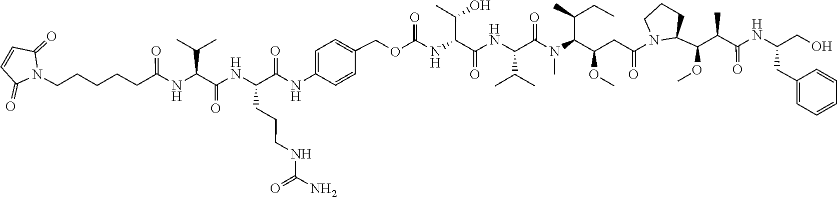 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](NC(=O)OCc1ccc(NC(=O)[C@H](CCCNC(N)=O)NC(=O)[C@@H](NC(=O)CCCCCN2C(=O)C=CC2=O)C(C)C)cc1)[C@H](C)O)C(C)C] |
| 2.22 | 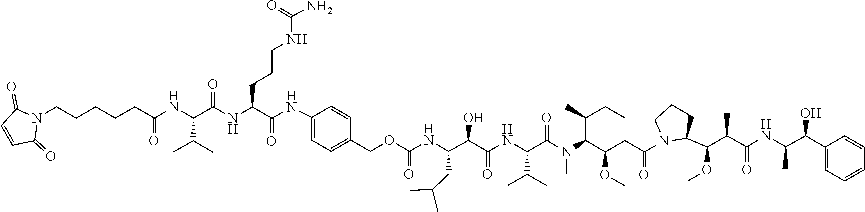 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](O)[C@H](CC(C)C)NC(=O)OCc1ccc(NC(=O)[C@H](CCCNC(N)=O)NC(=O)[C@@H](NC(=O)CCCCCN2C(=O)C=CC2=O)C(C)C)cc1)C(C)C] |
| 2.23 | 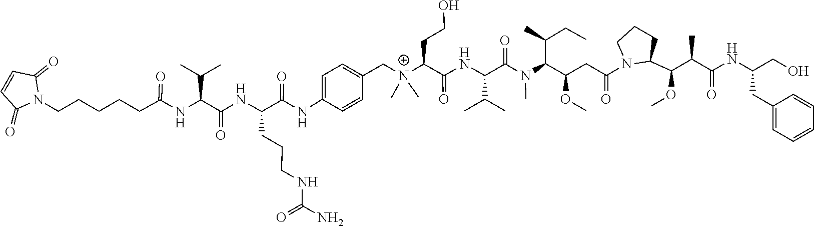 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CCO)[N+](C)(C)Cc1ccc(NC(=O)[C@H](CCCNC(N)=O)NC(=O)[C@@H](NC(=O)CCCCCN2C(=O)C=CC2=O)C(C)C)cc1)C(C)C] |
| 2.24 | 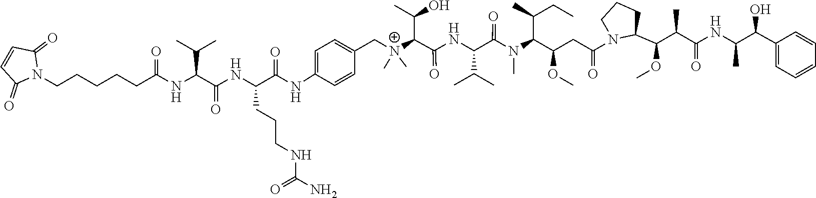 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H]([C@@H](C)O)[N+](C)(C)Cc1ccc(NC(=O)[C@H](CCCNC(N)=O)NC(=O)[C@@H](NC(=O)CCCCCN2C(=O)C=CC2=O)C(C)C)cc1)C(C)C] |
| 2.25 | 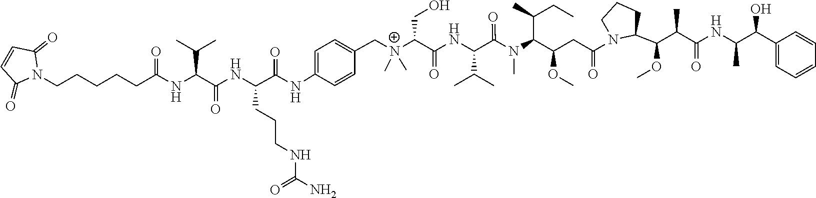 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](CO)[N+](C)(C)Cc1ccc(NC(=O)[C@H](CCCNC(N)=O)NC(=O)[C@@H](NC(=O)CCCCCN2C(=O)C=CC2=O)C(C)C)cc1)C(C)C] |
| 2.26 | 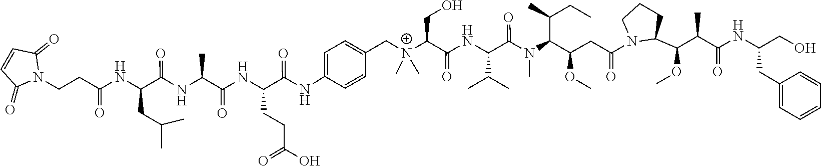 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)[N+](C)(C)Cc1ccc(NC(=O)[C@H](CCC(=O)O)NC(=O)[C@H](C)NC(=O)[C@@H](CC(C)C)NC(=O)CCN2C(=O)C=CC2=O)cc1)C(C)C] |
| 2.27 | 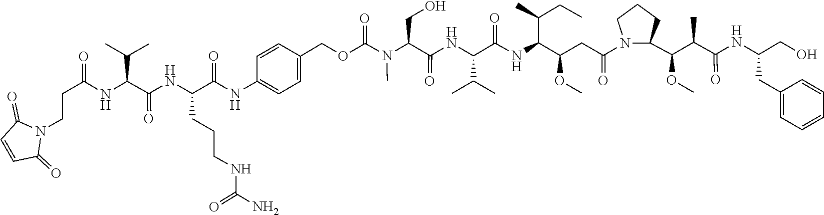 [SMILES: CC[C@H](C)[C@H](NC(=O)[C@@H](NC(=O)[C@H](CO)N(C)C(=O)OCc1ccc(NC(=O)[C@H](CCCNC(N)=O)NC(=O)[C@@H](NC(=O)CCN2C(=O)C=CC2=O)C(C)C)cc1)C(C)C)[C@@H](CC(=O)N1CCC[C@H]1[C@H](OC)[C@@H](C)C(=O)N[C@H](CO)Cc1ccccc1)OC] |
| 2.28 | 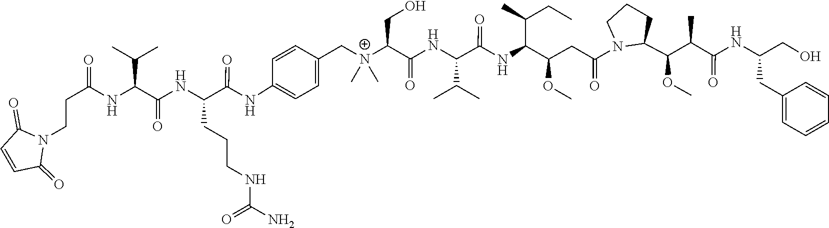 [SMILES: CC[C@H](C)[C@H](NC(=O)[C@@H](NC(=O)[C@H](CO)[N+](C)(C)Cc1ccc(NC(=O)[C@H](CCCNC(N)=O)NC(=O)[C@@H](NC(=O)CCN2C(=O)C=CC2=O)C(C)C)cc1)C(C)C)[C@@H](CC(=O)N1CCC[C@H]1[C@H](OC)[C@@H](C)C(=O)N[C@H](CO)Cc1ccccc1)OC] |
| 2.29 | 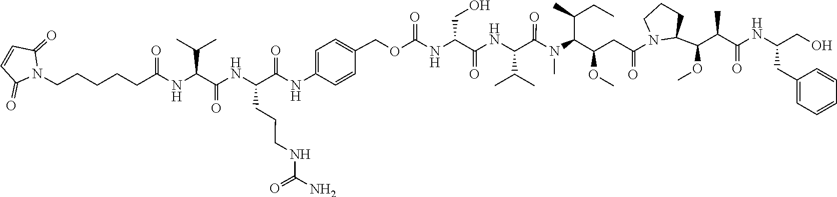 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](CO)NC(=O)OCc1ccc(NC(=O)[C@H](CCCNC(N)=O)NC(=O)[C@@H](NC(=O)CCCCCN2C(=O)C=CC2=O)C(C)C)cc1)C(C)C] |

### Ligand-Drug Conjugate Compounds

In context of Ligand-Drug Conjugate compounds—the assembly is described by the component groups as described for Drug-Linker compounds, with the exception of the Stretcher Unit Z and the Ligand Unit L. The Stretcher Unit Z is coordinated to the Ligand Unit L in Ligand-Drug Conjugate compounds, as described below. While some procedures for the preparation of Ligand-Drug Conjugate compounds are described herein, the order of assembly and the general conditions to prepare the compounds will be well understood by one of skill in the art.

In some embodiments, a Ligand-Drug Conjugate compound comprises an auristatin compound of Formula (I), or any subformula thereof, a Linker Unit (Q) comprising a Releasable Linker (RL) that is other than a Glycoside (e.g., Glucuronide) Unit through which the Ligand Unit is connected to the conjugated auristatin compound, and a Ligand Unit (L). The Linker Unit comprises, in addition to RL, a Stretcher Unit (Z) connected to the Ligand Unit and is capable of (directly or indirectly) connecting the RL to the Ligand Unit. In some embodiments, a Parallel Connector Unit (B) is present when it is desired to add a Partitioning Agent (S*) as a side chain appendage. In any one of those embodiments, a Connector Unit (A) is present when it is desirable to add more distance between the Stretcher Unit and RL.

In some embodiments, a Ligand-Drug Conjugate compound comprises an auristatin compound of Formula (I), or any subformula thereof, and a Linker Unit (Q), wherein Q comprises a Releasable Linker (RL) that is a Glycoside (e.g., Glucuronide) Unit, directly attached to a Stretcher Unit (Z) or indirectly to Z through attachment to intervening component(s) of the Ligand-Drug Conjugate compound's Linker Unit (i.e., A, S* and/or B(S*)), wherein Z forms a covalent bond to a targeting agent (e.g., a Ligand Unit).

In another group of embodiments, a Ligand-Drug Conjugate compound comprises an auristatin of Formula (I), or any subformula thereof, a Linker Unit (Q), wherein Q comprises a Releasable Linker (RL) that is other than a Glycoside (e.g., Glucuronide) Unit (RL), directly attached to a Stretcher Unit (Z) or indirectly to Z through attachment to intervening component(s) of the Ligand-Drug Conjugate compound's Linker Unit (i.e., A, S* and/or B(S*)), wherein Z forms a covalent bond to a targeting agent (e.g., a Ligand Unit).

In some embodiments, the Ligand-Drug Conjugate compound has the formula:

L-(Q-D)ₚ

or a pharmaceutically acceptable salt thereof, wherein
L is a Ligand Unit;
Q is a Linker Unit selected from the group consisting of:
   (i) Z′-A-RL-,
   (ii) Z′-A-RL-Y—,
   (iii) Z′-A-S*—RL-,
   (iv) Z′-A-S*—RL-Y—,
   (v) Z′-A-B(S*)—RL-,
   (vi) Z′-A-B(S*)—RL-Y—,
   (vii) Z′-A-,
   (viii) Z′-A-S*—W—,
   (ix) Z′-A-B(S*)—W—,
   (x) Z′-A-S*—W-RL-, and
   (xi) Z′-A-B(S*)—W-RL-;
Z′ is a Stretcher Unit precursor;
A is a bond or a Connector Unit;
B is a Parallel Connector Unit;
S* is a Partitioning Agent;
RL is a Releasable Linker;
W is a Amino Acid Unit;
Y is a Spacer Unit; and
D is a Drug Unit of Formula (I′):

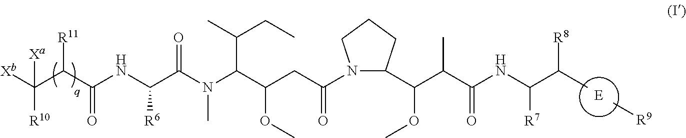

wherein
X^{b} is —NR²—^{#} or —N⁺R¹R⁵—^{#}, wherein the # denotes the point of attachment to Q, and X^{a} is

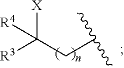

or
X^{a} and X^{b} are taken together with the carbon atom to which they are attached to form

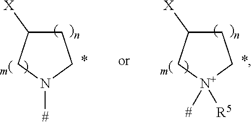

wherein the asterisk denotes the carbon atom of Formula (I) that bears the X^{a} and X^{b} groups, and the # denotes the point of attachment to Q;
R¹ and R⁵ are independently C₁-C₄ alkyl;
X is H, OH, —C(O)NR^{a}R^{b}, —S(O)₂R^{a}, —S(O)—R^{a} —S(O)₂NR^{a}R^{b}, —NHS(O)₂R^{a}, or —NHC(O)R^{a};
R², R³, R⁴, R¹⁰, R^{a}, and R^{b} are each independently H or C₁-C₄ alkyl;
R⁶ is C₁-C₄ alkyl optionally substituted with OH;
R⁷ is H, C₁-C₄ alkyl optionally substituted with OH, or 5-6 membered heteroaryl;
E is phenyl or 5-6 membered heteroaryl;
R⁸, R⁹, and R¹¹ are each independently H or OH;
n is 0, 1, 2, or 3;
m is 1, 2, 3, or 4;
q is 0 or 1; and
p is an integer ranging from 1 to 12,
wherein when X is H, at least two of R⁷, R⁸, R⁹, and R¹¹ comprise an OH moiety.

In some embodiments, provided herein are Ligand-Drug Conjugate compounds of formula L-(Q-D)ₚ, wherein the Drug Unit D has Formula (Iz*):

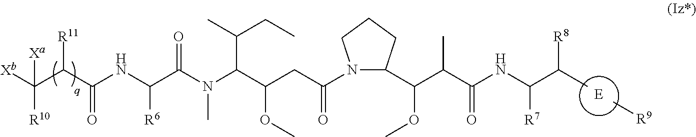

wherein
X^{b} is —NR²—^{#} or —N⁺R¹R⁵—^{#}, wherein the # denotes the point of attachment to Q, and X^{a} is

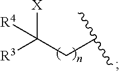

or
X^{a} and X^{b} are taken together with the carbon atom to which they are attached to form

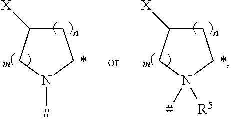

wherein the asterisk denotes the carbon atom of Formula (I) that bears the X^{a} and X^{b} groups, and the # denotes the point of attachment to Q;
R¹ and R⁵ are independently C₁-C₄ alkyl;
X is OH, —S(O)₂R^{a}, —S(O)—R^{a} —S(O)₂NR^{a}R^{b}, —NHS(O)₂R^{a}, or —NHC(O)R^{a};
R², R³, R⁴, R¹⁰, R^{a}, and R^{b} are each independently H or C₁-C₄ alkyl;
R⁶ is C₁-C₄ alkyl optionally substituted with OH;
R⁷ is H, C₁-C₄ alkyl optionally substituted with OH, or 5-6 membered heteroaryl;
E is phenyl or 5-6 membered heteroaryl;
R⁸, R⁹, and R¹¹ are each independently H or OH;
n is 0, 1, 2, or 3;
m is 1, 2, 3, or 4;
q is 0 or 1; and
p is an integer ranging from 1 to 12.

In some embodiments, the Ligand-Drug Conjugate compound has Formula (Ia*) or (Ia**):

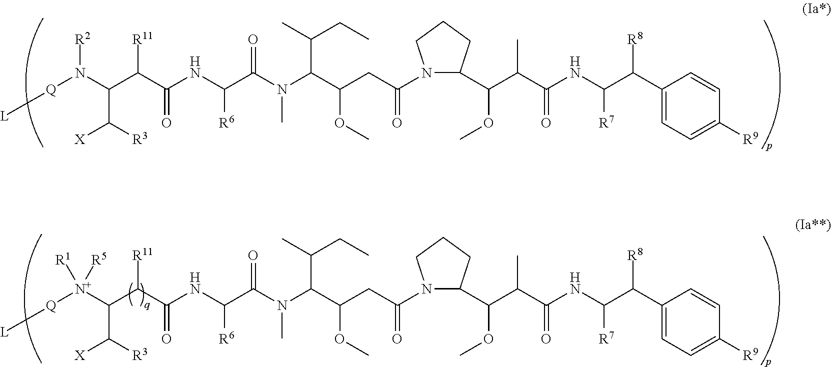

or a salt thereof, wherein
L is a Ligand Unit;
Q is a Linker Unit as described above;
p is an integer ranging from 1 to 12; and
the remaining variables are as described for Drug-Linker moieties of Formula (Ia′) or Formula (Ia″) above.

In some embodiments, the Ligand-Drug Conjugate compound has Formula (Ib*) or (Ib**):

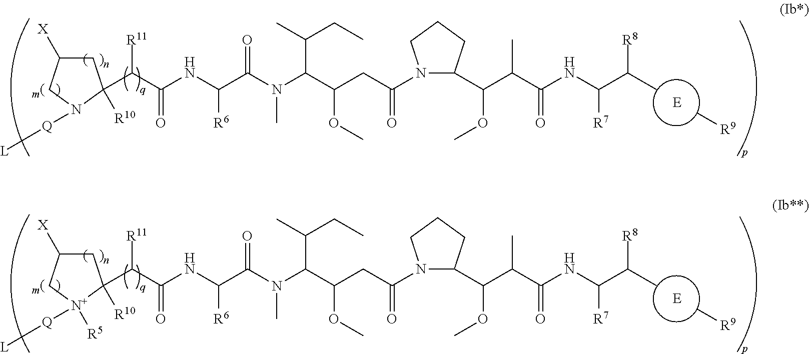

or a salt thereof, wherein
L is a Ligand Unit;
Q is a Linker Unit as described above;
p is an integer ranging from 1 to 12; and
the remaining variables are as described for Drug-Linker moieties of Formula (Ib′) or Formula (Ib″) above.

In some embodiments, the Ligand-Drug Conjugate compound has Formula (Ic*) or (Ic**):

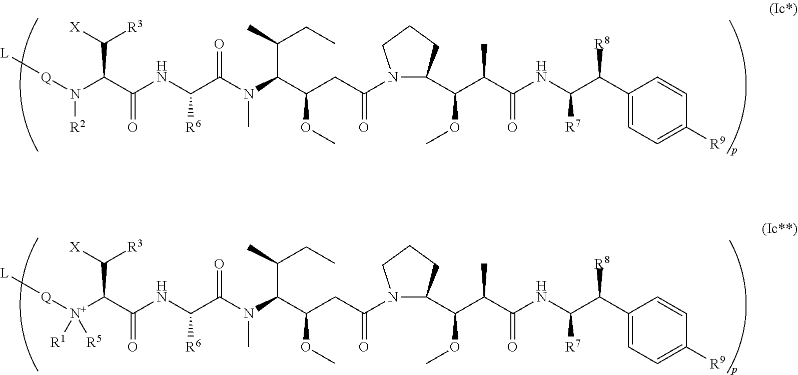

or a salt thereof, wherein
L is a Ligand Unit;
Q is a Linker Unit as described above;
p is an integer ranging from 1 to 12; and
the remaining variables are as described for Drug-Linker moieties of Formula (Ic′) or Formula (Ic″) above.

In some embodiments, the Ligand-Drug Conjugate compound has Formula (Id*) or (Id**):

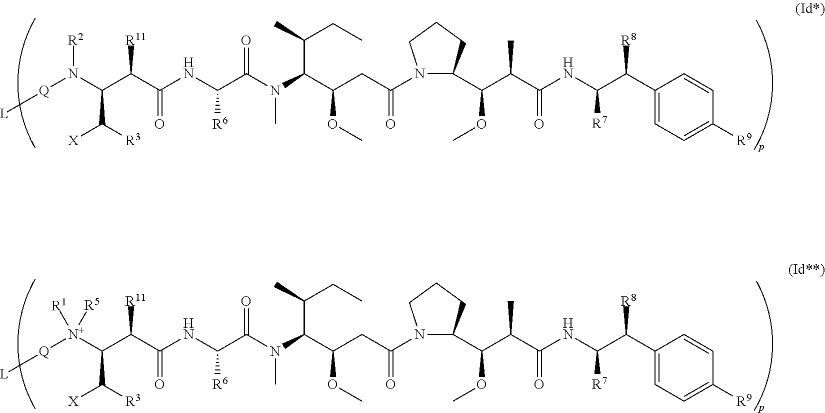

or a salt thereof, wherein
L is a Ligand Unit;
Q is a Linker Unit as described above;
p is an integer ranging from 1 to 12; and
the remaining variables are as described for Drug-Linker moieties of Formula (Id′) or Formula (Id″) above.

In some embodiments, the Ligand-Drug Conjugate compound has Formula (Ie*) or (Ie**):

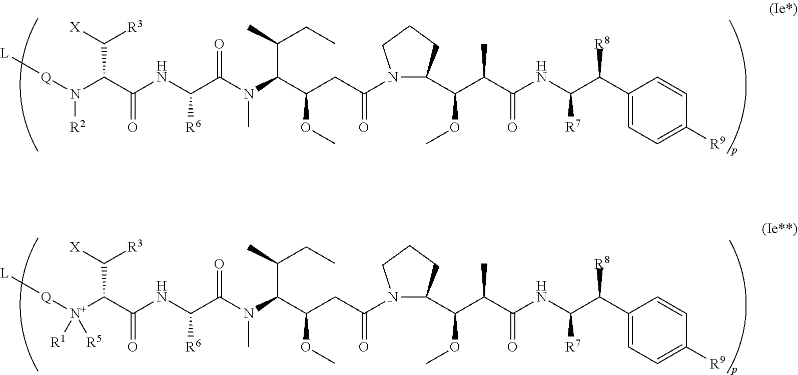

or a salt thereof, wherein
L is a Ligand Unit;
Q is a Linker Unit as described above;
p is an integer ranging from 1 to 12; and
the remaining variables are as described for Drug-Linker moieties of Formula (Ie′) or Formula (Ie″) above.

In some embodiments, the Ligand-Drug Conjugate compound has Formula (If*) or (If**):

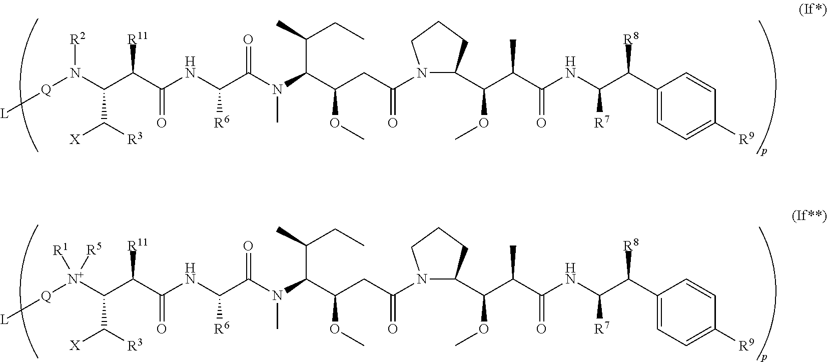

or a salt thereof, wherein
L is a Ligand Unit;
Q is a Linker Unit as described above;
p is an integer ranging from 1 to 12; and
the remaining variables are as described for Drug-Linker moieties of Formula (If) or Formula (If′) above.

In the context of the Ligand-Drug Conjugate compounds—the assembly is best described in terms of its component groups. While some procedures for the preparation of Ligand-Drug Conjugate compounds are described herein, the order of assembly and the general conditions to prepare the compounds will be well understood by one of skill in the art. The component groups of the Ligand-Drug Conjugate compounds described are in many cases identical to the component groups for Drug-Linker compounds as described above, including A, B, S*, RL, W, Y, and D. It is to be understood that embodiments are contemplated wherein the Drug Unit D of the Ligand-Drug Conjugates described herein is conforms to the description of Formula (I′), or any subformula thereof. Other component groups are described below.

### Stretcher Unit Z

Representative Stretcher Units of such embodiments include those having the structures of:

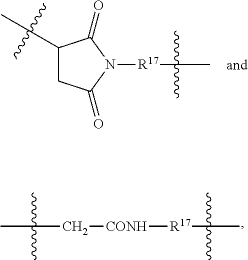

wherein the wavy line adjacent to R¹⁷ indicates attachment to the Parallel Connector Unit (B) or Connector Unit (A) if B is absent, or a Partitioning Agent (S*), if B is absent, the other wavy line indicates covalent attachment to a sulfur atom of a Ligand Unit, and R¹⁷ is —CH₂CH₂(OCH₂CH₂)ₖ—, —C₁-C₁₀ alkylene-, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—, wherein subscript k is an integer ranging from 1 to 36.

In some embodiments, the R¹⁷ group is optionally substituted by a Basic Unit (BU) such as an aminoalkyl moiety, e.g. —(CH₂)ₓNH₂, —(CH₂)ₓNHR^{a}, and —(CH₂)ₓNR^{a}₂, wherein subscript x is an integer of from 1-4 and each R^{a} is independently selected from the group consisting of C₁₋₆ alkyl and C₁₋₆ haloalkyl, or two R^{a} groups are combined with the nitrogen to which they are attached to form an azetidinyl, pyrrolidinyl or piperidinyl group.

An illustrative Stretcher Unit is that of Formula Za or Za-BU in which R¹⁷ is —CH₂CH₂(OCH₂CH₂)ₖ—, —C₁-C₁₀ alkylene-C(═O)—, —C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C3-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, or —(C₃-C₈ heterocyclo)-C₁-C₁₀ alkylene-C(═O), wherein subscript k is an integer ranging from 1 to 36:

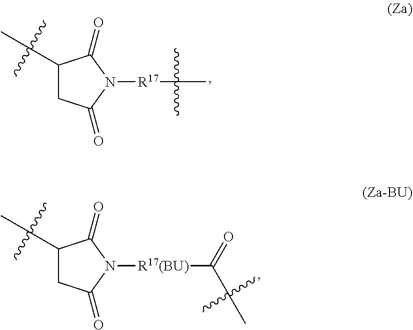

wherein the wavy line adjacent the carbonyl carbon atom indicates attachment to LP, B, A, or S* in the formulae above, depending on the presence or absence of A and/or B, and the other wavy line indicates covalent bonding of the succinimide ring carbon atom to a sulfur atom of a Ligand Unit. In some aspects, the basic amino functional group of the Basic Unit (BU) is protected by a protecting group during synthesis.

In some embodiments, Stretcher Units of formula Za and Za-BU are as follows:

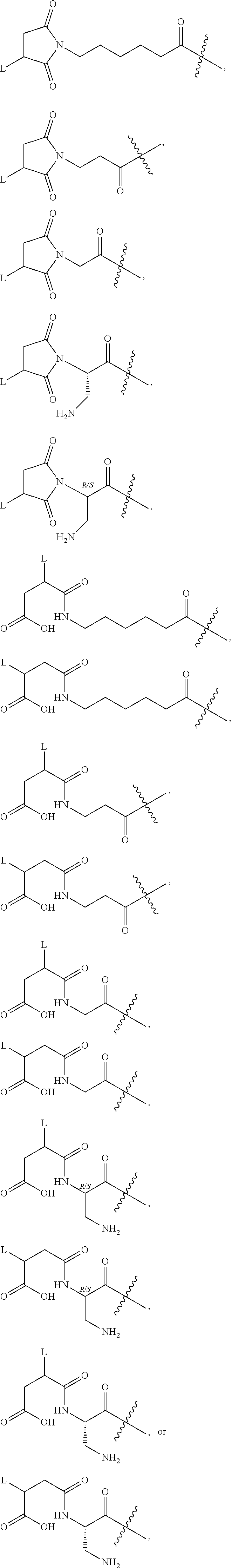

wherein the wavy line adjacent the carbonyl carbon atom indicates attachment to B, A, or S in the formulae above, depending on the presence or absence of A and/or B, and the other wavy line indicates covalent bonding of the succinimide ring carbon atom to a sulfur atom of a Ligand Unit.

It will be understood that a Ligand Unit-substituted succinimide may exist in hydrolyzed form(s). Those forms are exemplified below for hydrolysis of Za or Za-BU, wherein the structures representing the regioisomers from that hydrolysis have formula Zb and Zc or Zb-BU and Zc-BU.

Accordingly, in some embodiments, a Stretcher unit (Z) comprises a succinic acid-amide moiety represented by the following:

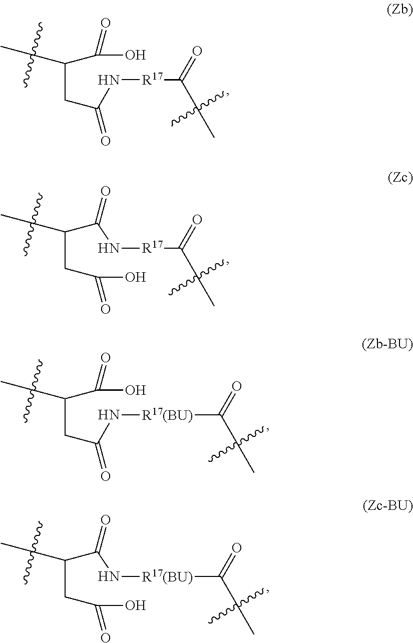

wherein the wavy line adjacent to the carbonyl carbon atom bonded to R¹⁷ and the wavy line adjacent to the carbon atom of the succinic acid-amide moiety is as defined for Za or Za-BU, depending on the presence or absence of A and/or B; and R¹⁷ is —C₁-C₈ alkylene-, wherein in Zb-BU and Zc-BU the alkylene is substituted by a Basic Unit (BU), wherein BU is —(CH₂)ₓNH₂, —(CH₂)ₓNHR^{a}, or —(CH₂)ₓN(R^{a})₂, wherein subscript x is an integer of from 1-4 and each R^{a} is independently selected from the group consisting of C₁₋₆ alkyl and C₁₋₆ haloalkyl, or both R^{a} together with the nitrogen to which they are attached define an azetidinyl, pyrrolidinyl or piperidinyl group.

In some embodiments, —Z-A- comprises a moiety derived from a maleimido-alkanoic acid moiety or an mDPR moiety. See, for example, see WO 2013/173337. In one group of embodiments, Z-A- is derived from a maleimido-propionyl moiety.

In some embodiments, a Stretcher unit (Z) comprises an succinic acid-amide moiety represented by the structure of formula Zb′, Zc′, (R/S)-Zb′-BU, (S)-Zb′-BU, (R/S)-Zc′-BU or (S)-Zc′-BU as follows:

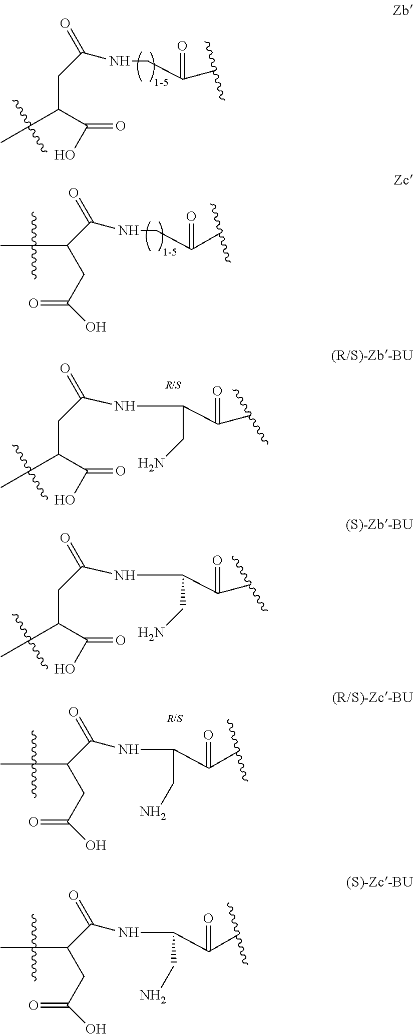

wherein the wavy lines are as defined for Za or Za-BU.

In some embodiments, a Stretcher unit (Z) comprises a succinimide moiety represented by the structure of

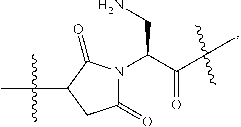 [SMILES: *C(=O)[C@H](CN)N1C(=O)CC(*)C1=O]

which may be generated from a maleimido-amino-propionyl (mDPR) analog (a 3-amino-2-(2,5-dioxo-2,5-dihydro-1H-pyrrol-1-yl)propanoic acid derivative), or comprises a succinic acid-amide moiety represented by the structure of:

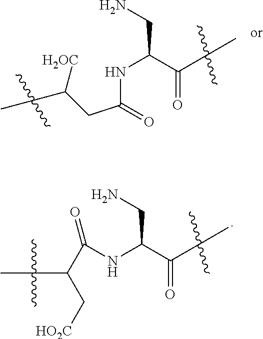

Illustrative Stretcher Units bonded to a Connector Unit (A) which comprise Za′, Zb′ or Zc′, in which —R¹⁷— of Za, Zb or Zc is —CH₂— or —CH₂CH₂—, or comprise Za′-BU, Zb′-BU or Zc′-BU in which —R¹⁷(BU)— of Za′-BU, Zb′-BU or Zc′-BU is —CH(CH₂NH₂)—, have the following structures:

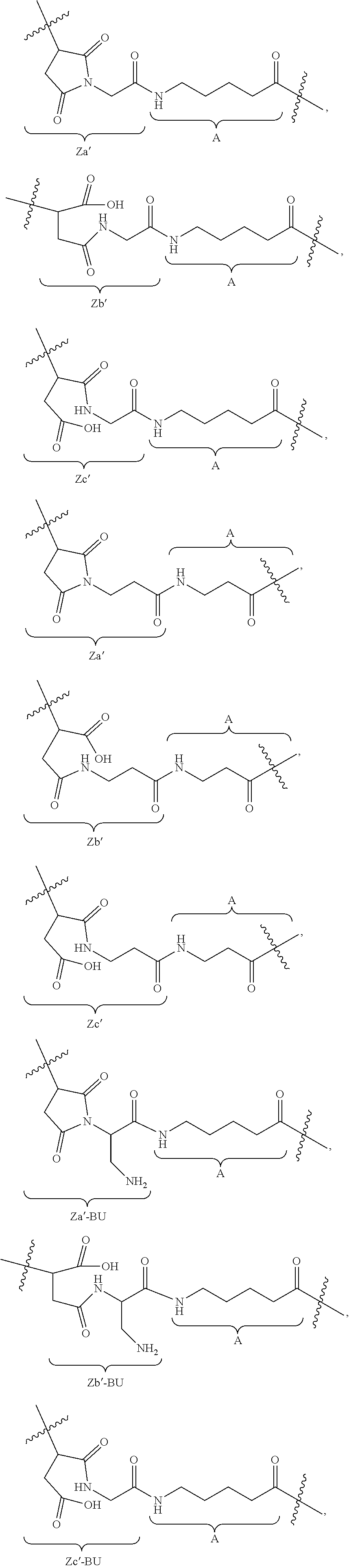

wherein the wavy lines are as defined for Za or Za-BU.

Other Stretcher Units bonded to a Ligand Unit (L) and a Connector Unit (A) have the structures above wherein A in any one of the above -Za′-A-, -Za′(BU)-A-, -Za′-A-, -Za′(BU)-A-, -Zb′-A-, -Zb′(BU)-A-, -Zb′-A-, -Zb′(BU)—, -Zc′-A- and Zc′(BU)-A- structures is replaced by a Parallel Connector Unit having the structure of:

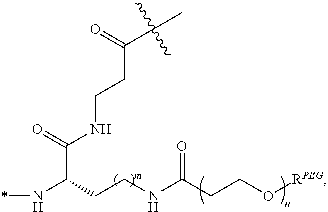

wherein subscript m ranges from 1 to 6; n ranges from 8 to 24; R^{PEG} is a PEG Capping Unit, preferably H, —CH₃, or —CH₂CH₂CO₂H, the asterisk (*) indicates covalent attachment to a Stretcher Unit corresponding in structure to formula Za, Za′, Zb′ or Zc′ and the wavy line indicates covalent attachment to the Releasable Linker (RL).

In another embodiment, the Stretcher Unit is attached to the Ligand Unit via a disulfide bond between a sulfur atom of the Ligand Unit and a sulfur atom of the Stretcher unit. A representative Stretcher Unit of this embodiment is depicted within the square brackets of Formula Zb:

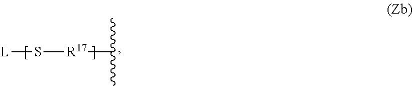

wherein the wavy line indicates attachment to the Parallel Connector Unit (B) or Connector Unit (A) if B is absent or a Partitioning Agent (S*), if A and B are absent and R¹⁷ is —CH₂CH₂(OCH₂CH₂)ₖ—, —C₁-C₁₀ alkylene-, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C10 alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—, wherein subscript k is an integer ranging from 1 to 36.

In yet another embodiment, the reactive group of a Stretcher Unit precursor contains a reactive site that can form a bond with a primary or secondary amino group of a Ligand Unit. Examples of these reactive sites include, but are not limited to, activated esters such as succinimide esters, 4-nitrophenyl esters, pentafluorophenyl esters, tetrafluorophenyl esters, anhydrides, acid chlorides, sulfonyl chlorides, isocyanates and isothiocyanates. Representative Stretcher Units of this embodiment are depicted within the square brackets of Formulas Zci, Zcii and Zciii:

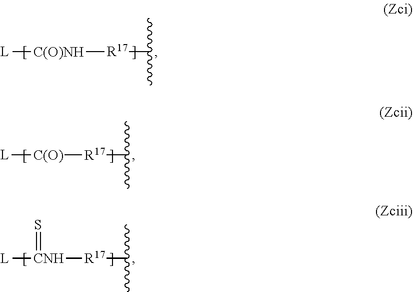

wherein the wavy line indicates attachment to the Parallel Connector Unit (B) or Connector Unit (A) if B is absent or a Partitioning Agent (S*), if A and B are absent and R¹⁷ is —CH₂CH₂(OCH₂CH₂)ₖ—, —C₁-C₁₀ alkylene-, C₁-C₁₀ heteroalkylene-, —C₃-C₈ carbocyclo-, —O—(C₁-C8 alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—, wherein subscript k is an integer ranging from 1 to 36.

In still other embodiments, the reactive group of the Stretcher Unit precursor contains a reactive nucleophile that is capable of reacting with an electrophile present on, or introduced to, a Ligand Unit. For example, in some aspects, a carbohydrate moiety on a targeting ligand is mildly oxidized using a reagent such as sodium periodate and the resulting electrophilic functional group (—CHO) of the oxidized carbohydrate is condensed with a Stretcher Unit precursor that contains a reactive nucleophile such as a hydrazide, an oxime, a primary or secondary amine, a hydrazine, a thiosemicarbazone, a hydrazine carboxylate, or an arylhydrazide such as those described by Kaneko, T. et al. (1991) Bioconjugate Chem. 2:133-41. Representative Stretcher Units of this embodiment are depicted within the square brackets of Formulas Zdi, Zdii, and Zdiii:

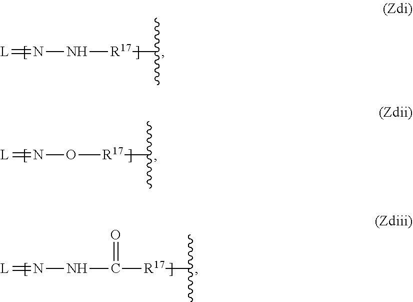

wherein the wavy line indicates attachment to the Parallel Connector Unit (B) or Connector Unit (A), or a Partitioning Agent (S*), if A and B are absent and R¹⁷ is —CH₂CH₂(OCH₂CH₂)ₖ—, —C₁-C₁₀ alkylene-, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—, wherein subscript k is an integer ranging from 1 to 36.

In some embodiments, provided herein is a Ligand-Drug Conjugate compound of Table 3, or a pharmaceutically acceptable salt thereof. Ligand-Drug Conjugate compounds corresponding to the compounds of Table 3 but having a succinic acid-amide moiety in place of the succinimide moiety are contemplated.

TABLE 3
Ligand-Drug Conjugate compounds

| # | Ligand-Drug Conjugate compound structure |
|---|---|
| 3.1 | 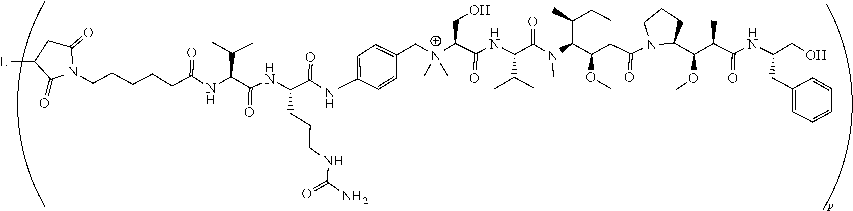 |
| 3.2 | 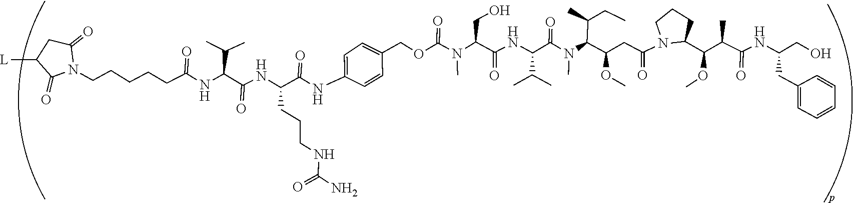 |
| 3.3 | 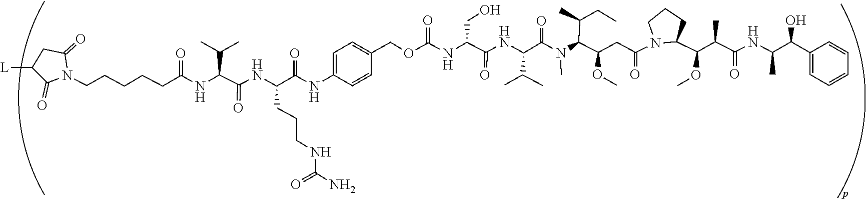 |
| 3.4 | 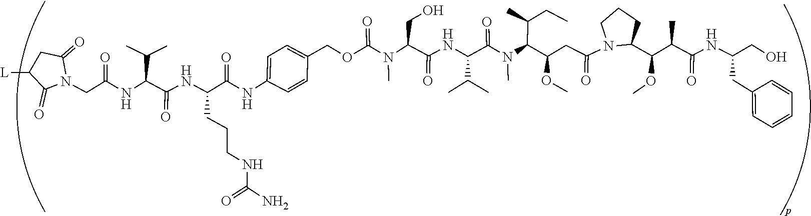 |
| 3.5 | 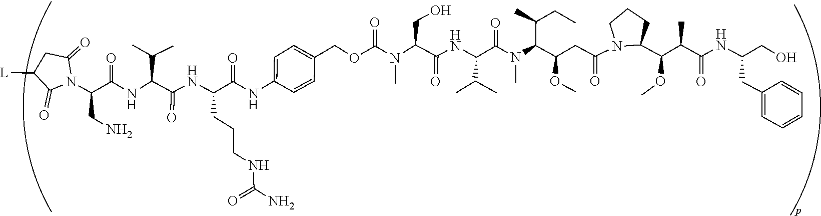 |
| 3.6 | 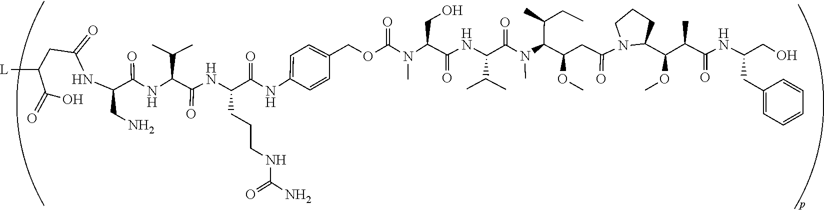 |
| 3.7 | 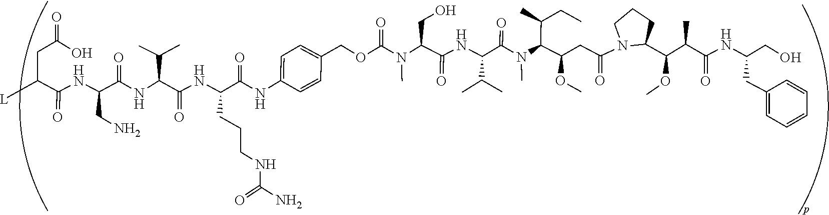 |
| 3.8 | 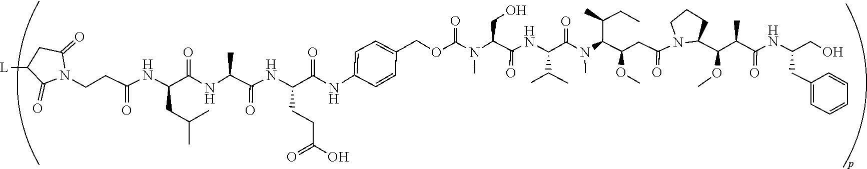 |
| 3.9 | 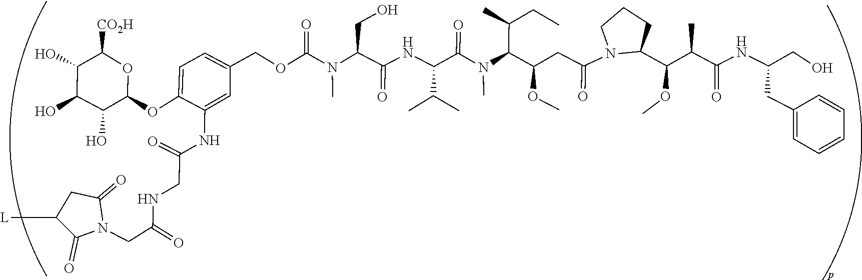 |
| 3.10 | 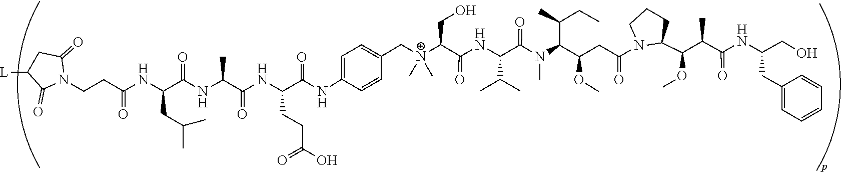 |
| 3.11 | 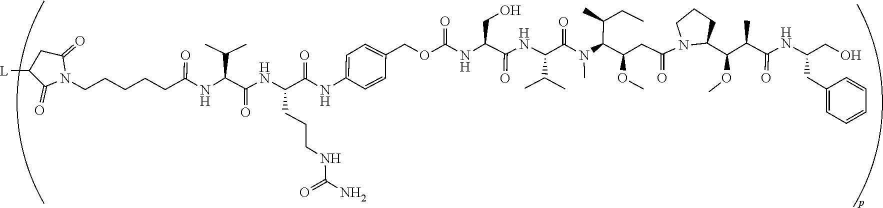 |
| 3.12 | 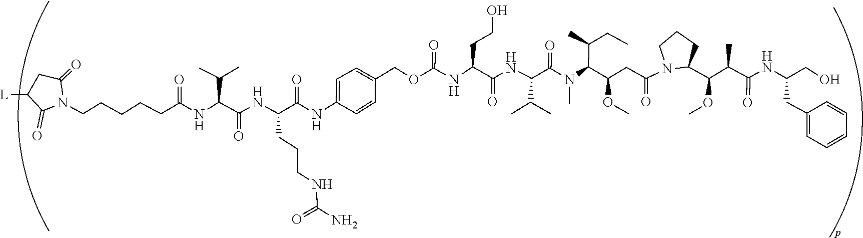 |
| 3.13 | 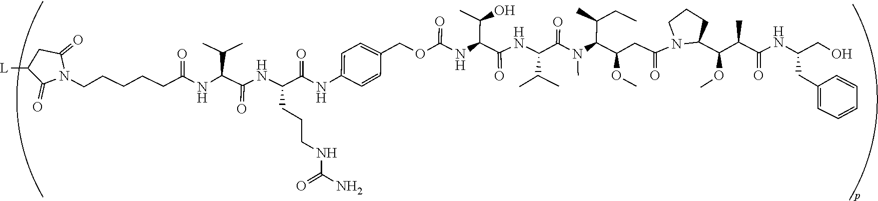 |
| 3.14 | 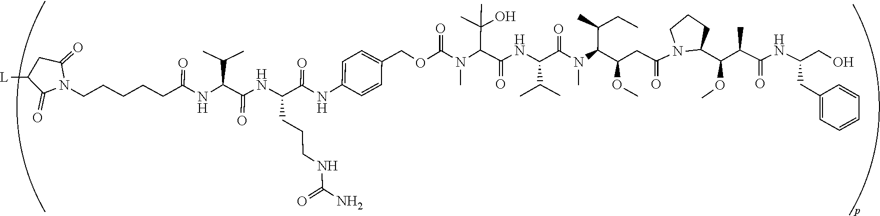 |
| 3.15 | 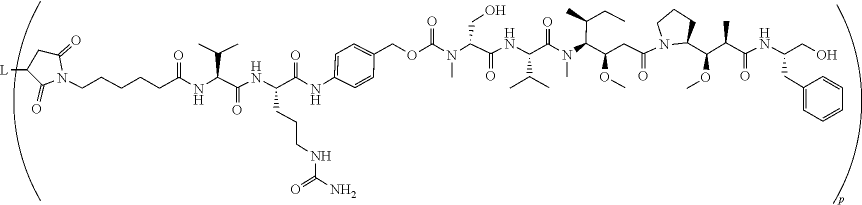 |
| 3.16 | 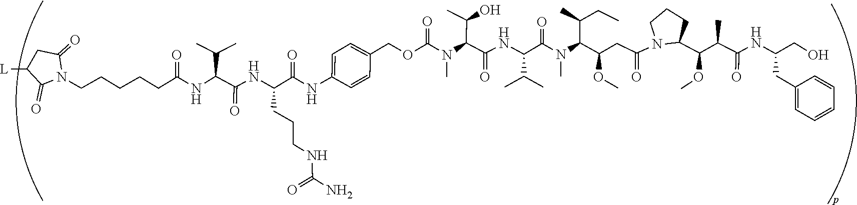 |
| 3.17 | 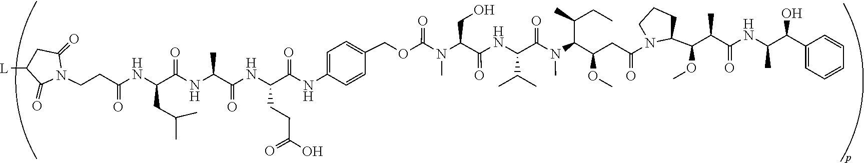 |
| 3.18 | 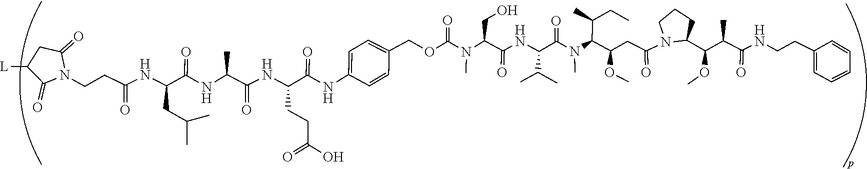 |
| 3.19 | 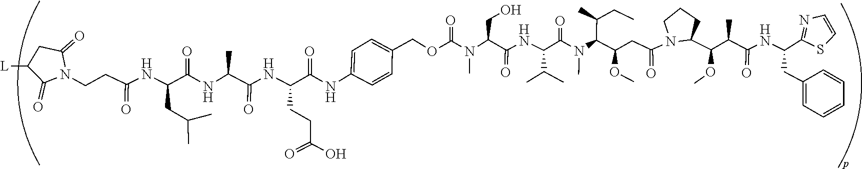 |
| 3.20 | 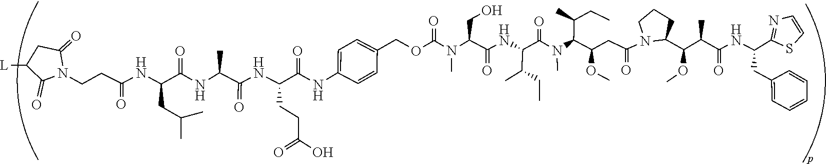 |
| 3.21 | 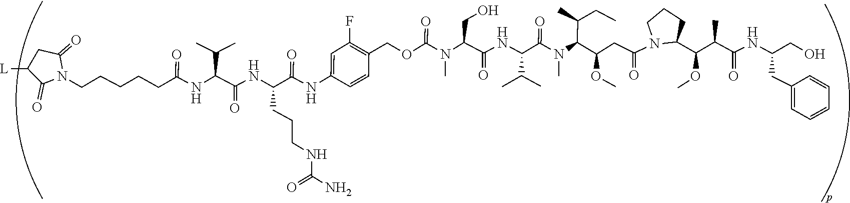 |
| 3.22 | 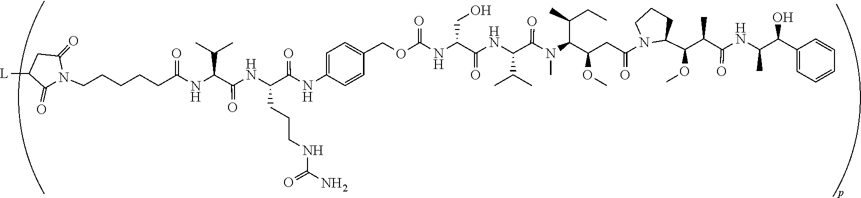 |
| 3.23 | 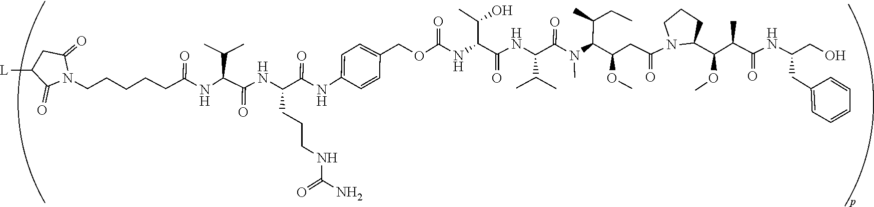 |
| 3.24 | 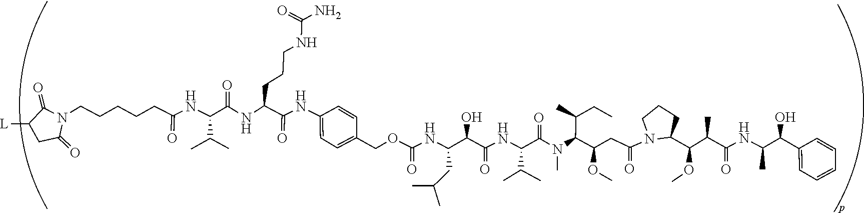 |
| 3.25 | 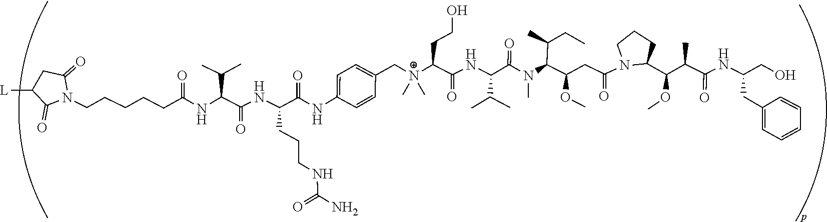 |
| 3.26 | 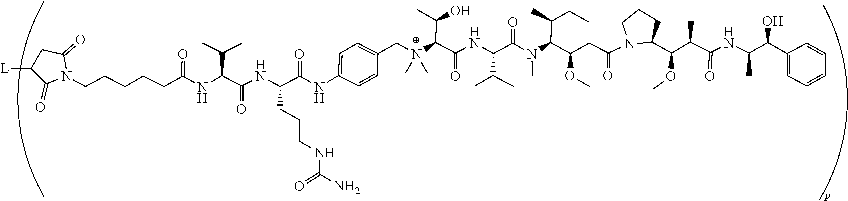 |
| 3.27 | 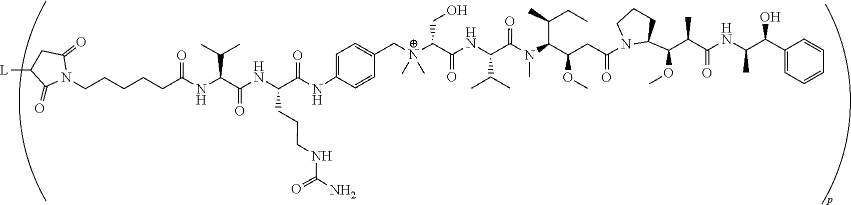 |
| 3.28 | 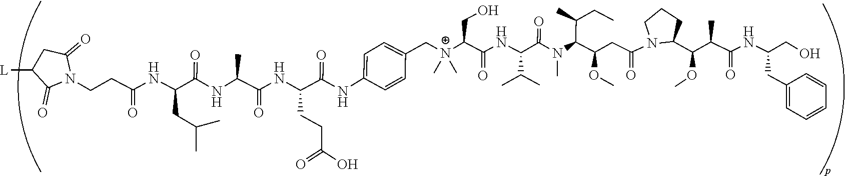 |
| 3.29 | 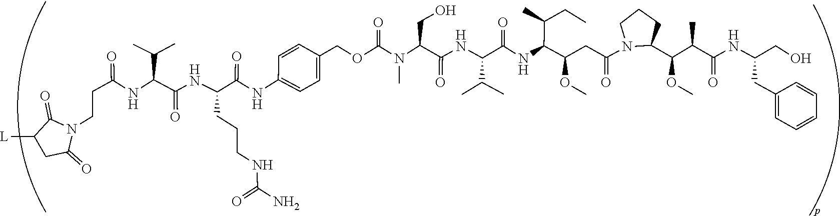 |
| 3.30 | 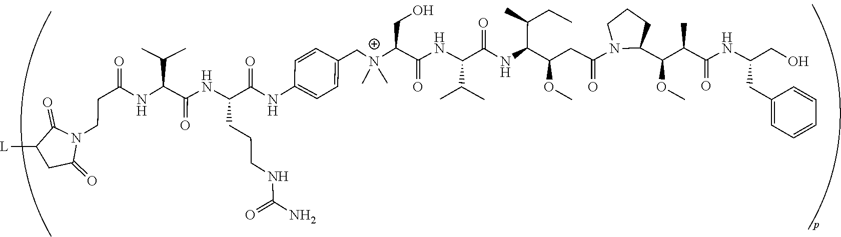 |

### Subscript p

Subscript p represents the number of Drug Linker moieties on a Ligand Unit of an individual Ligand-Drug Conjugate compound and is an integer ranging from 1 to 16, 1 to 12, 1 to 10, or 1 to 8. In any of the embodiments herein, there are 1, 2, 3, 4, 5, 6, 7, 8, 9, 10, 11, 12, 13, 14, 15, or 16 Drug Linker moieties conjugated to a Ligand Unit of an individual Ligand-Drug Conjugate compound.

In some embodiments, any of the structures and descriptions as described herein represent a population of individual Ligand-Drug Conjugate compounds substantially identical except for the number of Drug-Linker moieties bound to each Ligand Unit (i.e., a Ligand-Drug Conjugate composition) so that subscript p represents the average number of Drug-Linker moieties bound to the Ligand Units of the Ligand-Drug Conjugate composition. In that group of embodiments, subscript p is a number ranging from 1 to about 16, 1 to about 12, 1 to about 10, or 1 to about 8, from 2 to about 16, 2 to about 12, 2 to about 10, or 2 to about 8. In some aspects, p is about 2. In some aspects, p is about 4. In some aspects, p is about 8. In some aspects, p is about 16. In some aspects, p is 2. In some aspects, p is 4. In some aspects, p is 8. In some aspects, p is 16. In some embodiments, the value of subscript p refers to the average drug loading as well as the drug loading of the predominate Ligand-Drug Conjugate compound in the composition.

In some embodiments, conjugation will be via the reduced interchain disulfides and there will from 1 to about 8 Drug-Linker Compound molecules conjugated to a targeting agent that becomes a Ligand Unit. In some embodiments, conjugation will be via an introduced cysteine residue as well as reduced interchain disulfides and there will be from 1 to 10 or 1 to 12 or 1 to 14 or 1 to 16 Drug-Linker Compound moieties conjugated to a Ligand Unit. In some embodiments, conjugation will be via an introduced cysteine residue and there will be 2 or 4 Drug-Linker Compound molecules conjugated to a Ligand Unit.

### Ligand Unit L

In some embodiments of the invention, a Ligand Unit is present. The Ligand Unit (L-) is a targeting agent that specifically binds to a target moiety. In one group of embodiments, the Ligand Unit specifically and selectively binds to a cell component (a Cell Binding Agent) or to another target molecule of interest. The Ligand Unit acts to target and present the Drug Unit (such as one of Formula (I) or any subformula thereof) to the particular target cell population with which the Ligand Unit interacts due to the presence of its targeted component or molecule and allows for subsequent release of free drug within (i.e., intracellularly) or within the vicinity of the target cells (i.e., extracellularly). Ligand Units, L, include, but are not limited to, proteins, polypeptides and peptides. Suitable Ligand Units include, for example, antibodies, e.g., full-length antibodies and antigen binding fragments thereof, interferons, lymphokines, hormones, growth factors and colony-stimulating factors, vitamins, nutrient-transport molecules (such as, but not limited to, transferrin), or any other cell binding molecule or substance. In some embodiments, the Ligand Unit (L) is from an antibody or a non-antibody protein targeting agent.

In one group of embodiments a Ligand Unit is bonded to Q (a Linker Unit) which comprises a Glucuronide Releasable Linker. As noted above, in some aspects other linking components are present in the conjugates described herein to serve the purpose of providing additional space between the Drug Unit compound and the Ligand Unit (e.g., a Stretcher Unit and optionally a Connector Unit, A), or providing attributes to the composition to increases solubility (e.g., a Partitioning Agent, S*). In some of those embodiments, the Ligand Unit is bonded to Z of the Linker Unit via a heteroatom of the Ligand Unit. Heteroatoms that may be present on a Ligand Unit for that bonding include sulfur (in one embodiment, from a sulfhydryl group of a targeting ligand), oxygen (in one embodiment, from a carboxyl or hydroxyl group of a targeting ligand) and nitrogen, optionally substituted (in one embodiment, from a primary or secondary amine functional group of a targeting ligand or in another embodiment from an optionally substituted amide nitrogen). Those heteroatoms are present on the targeting ligand in the ligand's natural state, for example in a naturally occurring antibody, or are introduced into the targeting ligand via chemical modification or biological engineering.

In some embodiments, the Ligand Unit is an antibody.

Useful polyclonal antibodies are heterogeneous populations of antibody molecules derived from the sera of immunized animals. Useful monoclonal antibodies are homogeneous populations of antibodies to a particular antigenic determinant (e.g., a cancer or immune cell antigen, a protein, a peptide, a carbohydrate, a chemical, nucleic acid, or fragments thereof). A monoclonal antibody (mAb) to an antigen-of-interest can be prepared by using any technique known in the art which provides for the production of antibody molecules by continuous cell lines in culture.

Useful monoclonal antibodies include, but are not limited to, human monoclonal antibodies, humanized monoclonal antibodies, or chimeric human-mouse (or other species) monoclonal antibodies. The antibodies include full-length antibodies and antigen binding fragments thereof. Human monoclonal antibodies may be made by any of numerous techniques known in the art (e.g., Teng et al., 1983, Proc. Natl. Acad. Sci. USA. 80:7308-7312; Kozbor et al., 1983, Immunology Today 4:72-79; and Olsson et al., 1982, Meth. Enzymol. 92:3-16).

In some embodiments, an antibody includes a functionally active fragment, derivative or analog of an antibody that binds specifically to target cells (e.g., cancer cell antigens) or other antibodies bound to cancer cells or matrix. In this regard, “functionally active” means that the fragment, derivative or analog is able to bind specifically to target cells. To determine which CDR sequences bind the antigen, synthetic peptides containing the CDR sequences are typically used in binding assays with the antigen by any binding assay method known in the art (e.g., the Biacore assay) (See, e.g., Kabat et al., 1991, Sequences of Proteins of Immunological Interest, Fifth Edition, National Institute of Health, Bethesda, Md; Kabat E et al., 1980, J. Immunology 125(3):961-969).

Additionally, recombinant antibodies, such as chimeric and humanized monoclonal antibodies, comprising both human and non-human portions, which are typically obtained using standard recombinant DNA techniques, are useful antibodies. A chimeric antibody is a molecule in which different portions are derived from different animal species, such as for example, those having a variable region derived from a murine monoclonal and a constant region derived from a human immunoglobulin. See, e.g., U.S. Pat. Nos. 4,816,567; and 4,816,397, which are incorporated herein by reference in their entireties. Humanized antibodies are antibody molecules from non-human species having one or more CDRs from the non-human species and a framework region from a human immunoglobulin molecule. See, e.g., U.S. Pat. No. 5,585,089, which is incorporated herein by reference in its entirety. Such chimeric and humanized monoclonal antibodies can be produced by recombinant DNA techniques known in the art, for example using methods described in International Publication No. WO 87/02671; European Patent Publication No. 0 184 187; European Patent Publication No. 0 171 496; European Patent Publication No. 0 173 494; International Publication No. WO 86/01533; U.S. Pat. No. 4,816,567; European Patent Publication No. 012 023; Berter et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Cancer. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, BioTechniques 4:214; U.S. Pat. No. 5,225,539; Jones et al., 1986, Nature 321: 522-525; Verhoeyan et al., 1988, Science 239:1534; and Beidler et al., 1988, J. Immunol. 141:4053-4060; each of which is incorporated herein by reference in its entirety.

In some embodiments, an antibody is a completely human antibody. In some embodiments, an antibody is produced using transgenic mice that are incapable of expressing endogenous immunoglobulin heavy and light chain genes, but which are capable of expressing human heavy and light chain genes.

In some embodiments, an antibody is an intact or fully-reduced antibody. The term ‘fully-reduced’ is meant to refer to an antibody in which all four inter-chain disulfide linkages have been reduced to provide eight thiols that can be attached to a linker (L).

Attachment to an antibody can be via thioether linkages from native and/or engineered cysteine residues, or from an amino acid residue engineered to participate in a cycloaddition reaction (such as a click reaction) with the corresponding linker intermediate. See, e.g., Maerle, et al., PLOS One 2019: 14(1); e0209860. In some embodiments, an antibody is an intact or fully-reduced antibody, or is an antibody bearing engineered an cysteine group that is modified with a functional group that can participate in, for example, click chemistry or other cycloaddition reactions for attachment of other components of the ADC as described herein (e.g., Diels-Alder reactions or other [3+2] or [4+2] cycloadditions). See, e.g., Agard, et al., J. Am. Chem. Soc. Vol. 126, pp. 15046-15047 (2004); Laughlin, et al., Science, Vol. 320, pp. 664-667 (2008); Beatty, et al., ChemBioChem, Vol. 11, pp. 2092-2095 (2010); and Van Geel, et al., Bioconjug. Chem. Vol. 26, pp.2233-2242 (2015).

Antibodies that bind specifically to a cancer or immune cell antigen are available commercially or produced by any method known to one of skill in the art such as, e.g., chemical synthesis or recombinant expression techniques. The nucleotide sequences encoding antibodies that bind specifically to a cancer or immune cell antigen are obtainable, e.g., from the GenBank database or similar database, literature publications, or by routine cloning and sequencing.

In some embodiments, the antibody can be used for the treatment of a cancer (e.g., an antibody approved by the FDA and/or EMA). Antibodies that bind specifically to a cancer or immune cell antigen are available commercially or produced by any method known to one of skill in the art such as, e.g., recombinant expression techniques. The nucleotide sequences encoding antibodies that bind specifically to a cancer or immune cell antigen are obtainable, e.g., from the GenBank database or similar database, literature publications, or by routine cloning and sequencing.

In some cases, the antibody has a mutation or post-translational modification which affects non-Fab mediated uptake. Peripheral immune cells, which are highly responsive to TLR7/8 agonists can facilitate strong, systemic responses when activated through non-antigen specific, Fc-mediated uptake of the TLR7/8 antibody drug conjugate which may be harmful or otherwise undesirable. In such cases, the antibody can have an effector function-diminishing mutation, such as L234A/L235A, D265A/N297A, D270A, K322A, P329A, P329G or a combination thereof, which diminishes non-immune uptake (e.g., FcgR-mediated uptake).

In some embodiments, an antibody can be configured to bind to a surface antigen of a cell. The antibody, or a complex comprising the antibody, can be configured to internalize within a cell upon binding to the surface antigen. For example, the antibody or an ADC comprising the antibody can be configured to endocytose upon binding to a surface antigen of a cell. In some embodiments, the antibody (or an ADC comprising the antibody) is configured to internalize within a cancer cell. In some embodiments, the antibody (or an ADC comprising the antibody) is configured to internalize within an immune cell. In some embodiments, the immune cell is a tumor associated macrophage. In some embodiments, the surface antigen is a receptor or a receptor complex (e.g., expressed on lymphocytes). In some embodiments, the receptor or receptor complex comprises an immunoglobulin gene superfamily member, a TNF receptor superfamily member, an integrin, a cytokine receptor, a chemokine receptor, a major histocompatibility protein, a lectin, or a complement control protein or other immune cell expressed surface receptor.

In some embodiments, an antibody is configured to bind specifically to a cancer cell antigen. In some embodiments, an antibody is configured to bind specifically to an immune cell antigen. In some embodiments, the immune cell antigen is a tumor associated macrophage antigen. In some embodiments, an antibody is configured to bind specifically to EphA2. It will be understood that the antibody component in an ADC is an antibody in residue form such that “Ab” in the ADC structures described herein incorporates the structure of the antibody.

Non-limiting examples of antibodies that can be used for treatment of cancer and antibodies that bind specifically to tumor associated antigens are disclosed in Franke, A. E., Sievers, E. L., and Scheinberg, D. A., “Cell surface receptor-targeted therapy of acute myeloid leukemia: a review” Cancer Biother Radiopharm. 2000, 15, 459-76; Murray, J. L., “Monoclonal antibody treatment of solid tumors: a coming of age” Semin Oncol. 2000, 27, 64-70; Breitling, F., and Dubel, S., Recombinant Antibodies, John Wiley, and Sons, New York, 1998, each of which is hereby incorporated by reference in its entirety.

Non-limiting examples of antigens which antibodies of the present disclosure may target include ADAM12 (e.g., Catalog #14139-1-AP); ADAM9 (e.g., IMGC936); AFP (e.g., ThermoFisher Catalog #PA5-25959); AGR2 (e.g., ThermoFisher Catalog #PA5-34517); AKAP-4 (e.g., Catalog #PA5-52230); ALK (e.g., DLX521); ALPP (e.g., Catalog #MA5-15652); ALPPL2 (e.g., Catalog #PA5-22336); AMHR2 (e.g., ThermoFisher Catalog #PA5-13902); androgen receptor (e.g., ThermoFisher Catalog #MA5-13426); ANTXR1 (e.g., Catalog #MA1-91702); ANXA1 (e.g., Catalog #71-3400); ARTN (e.g., ThermoFisher Catalog #PA5-47063); ASCT2 (e.g., idactamab); Ax1 (e.g., BA3011; tilvestamab); B7-DC (e.g., Catalog #PA5-20344); B7-H3 (e.g., enoblituzumab, omburtamab, MGD009, MGC018, DS-7300); B7-H4 (e.g., Catalog #14-5949-82); B7-H6 (e.g., Catalog #12-6526-42); B7-H7; BAFF-R (e.g., Catalog #14-9117-82); BCMA; BCR-ABL; BMPR2; BORIS; C4.4a; CanAg; C5 complement (e.g., BCD-148; CAN106); CA-125; CA19-9 (e.g., AbGn-7; MVT-5873); CA9 (e.g., girentuximab); CALCR (see, e.g., International Publication No. WO 2015077826); CAMPATH-1 (e.g., alemtuzumab; ALLO-647; ANT1034); carcinoembryonic antigen (e.g., arcitumomab; cergutuzumab; amunaleukin; labetuzumab); CCNB1; CD112 (see, e.g., U.S. Publication No. 20100008928); CD115 (e.g., axatilimab; cabiralizumab; emactuzumab); CD123 (e.g., BAY-943; CSL360); CD137 (e.g., ADG106; CTX-471); CD138; CD142; CD166; CD147 (e.g., gavilimomab; metuzumab); CD155 (e.g., U.S. Publication No. 2018/0251548); CD19 (e.g., ALLO-501); CD20 (e.g., divozilimab; ibritumomab tiuxetan); CD24 (see, e.g., U.S. Pat. No. 8,614,301); CD244 (e.g., R&D AF1039); CD247 (e.g., AFM15); CD27 (e.g., varlilumab); CD274 (e.g., adebrelimab; atezolizumab; garivulimab); CD3 (e.g., otelixizumab; visilizumab); CD30 (e.g., iratumumab); CD33 (e.g., lintuzumab; BI 836858; AMG 673); CD288; CD352 (e.g., SGN-CD352A); CD37 (e.g., lilotomab; GEN3009); CD38 (e.g., felzartamab; AMG 424); CD3D; CD3E (e.g., foralumab; teplizumab); CD3G; CD45 (e.g., apamistamab); CD47 (e.g., letaplimab; magrolimab); CD48 (e.g., SGN-CD48A); CD5 (e.g., MAT 304; zolimomab aritox); CD56; CD59; CD70 (e.g., cusatuzumab); CD74 (e.g., milatuzumab); CD79A (see, e.g., International Publication No. WO 2020252110); CD79b; CD96; CD97; CD-262 (e.g., tigatuzumab); CDCP1 (e.g., RG7287); CDH17 (see, e.g., International Publication No. WO 2018115231); CDH3 (e.g., PCA062); CDH6 (e.g., HKT288); CEACAMI; CEACAM5; CEACAM6; CLDN1 (e.g., INSERM anti-Claudin-1); CLDN16; CLDN18.1 (e.g., zolbetuximab); CLDN18.2 (e.g., zolbetuximab); CLDN19; CLDN2 (see, e.g., International Publication No. WO 2018123949); CLEC12A (e.g., tepoditamab); CS1; CLPTM1L; CSPG4 (e.g., U.S. Pat. No. 10,822,427); CXCR4 (e.g., ulocuplumab); CYP1B1; c-Met; DCLK1 (see, e.g., International Publication No. WO 2018222675); DDR1; de2-7 EGFR (e.g., MAb 806); DLL-3; DPEP1; DPEP3; DPP4; DR4 (e.g., mapatumumab); DSG2 (see, e.g., U.S. Pat. No. 10,836,823); EGF; EGFR; endosialin (e.g., ontuxizumab); ENPP1; EPCAM (e.g., adecatumumab); EPHA receptors; EPHA2; ERBB2 (e.g., trastuzumab); ERBB3; ERVMER34_1; ETV6-AML (e.g., Catalog #PA5-81865); FAS; FasL; Fas-related antigen 1; FBP; FGFR1 (e.g., RG7992); FGFR2 (e.g., aprutumab); FGFR3 (e.g., vofatamab); FGFR4 (e.g., MM-161); FLT3 (e.g., 4G8SDIEM); FN; FN1; FOLR1 (e.g., farletuzumab); FRa; FSHR; FucGM1 (e.g., BMS-986012); FZD5; FZD8; G250; GAGE; GCC; GD2 (e.g., dinutuximab); GD3 (e.g., mitumomab); GITR (e.g., ragifilimab); GloboH; GM2 (e.g., BIW-8962); GM3 (e.g., racotumomab); gp100; GPA33 (e.g., KRN330); GPC3 (e.g., codrituzumab); gpNMB (e.g., glembatumumab); GPR87; GUCY2C (e.g., indusatumab); HAS3; HAVCR2; HLA-E; HLA-F; HLA-G (e.g., TTX-080); HPV E6 E7; hTERT; ICAM1; IDO1; IFNAR1 (e.g., faralimomab); IFNAR2; IL13Ra2; ILIRAP (e.g., nidanilimab); IL-21R (e.g., PF-05230900); IL-5R (e.g., benralizumab); ITGAV (e.g., abituzumab); ITGB6; ITGB8; KISSIR; L1CAM (e.g., JCAR023); LAG-3 (e.g., encelimab); LAMP1; LCK; legumain; LMP2; LY6G6D (e.g., PA5-23303); LY9 (e.g., PA5-95601); LYPD1 (e.g., ThermoFisher Catalog #PA5-26749); MAD-CT-1; MAD-CT-2; MAGEA1 (e.g., Catalog #MA5-11338); MAGEA3 (e.g., ThermoFisher Catalog #60054-1-IG); MAGEA4 (e.g., Catalog #MA5-26117); MAGEC2 (e.g., ThermoFisher Catalog #PA5-64010); MELTF (e.g., ThermoFisher Catalog #H00004241-M04A); MerTk (e.g., DS5MMER, Catalog #12-5751-82); a metalloproteinase; MFSDi3A; MICA (e.g., 1E2C8, Catalog #66384-1-IG); MICB (e.g., Catalog #MA5-29422); Mincle (e.g., OTI2A8, Catalog #TA505101); MLANA (e.g., Catalog #MA5-15237); ML-IAP (e.g., 88C570, ThermoFisher Catalog #40958); MSLN (e.g., 5B2, Catalog #MA5-11918); MUC1 (e.g., MHI (CT2), ThermoFisher Catalog #MA5-11202); MUC5AC (e.g., 45M1, Catalog #MA5-12178); MYCN (e.g., NCM-II 100, ThermoFisher Catalog #MA1-170); NA17; NCAM1 (e.g., ThermoFisher Catalog #MA5-11563); Nectin-4 (e.g., enfortumab); NOX1 (e.g., Catalog #PA5-103220); NT5E (e.g., 7G2, ThermoFisher Catalog #41-0200); NY-BR-I (e.g., NY-BR-I No. 2, Catalog #MA5-12645); NY-ESO-I (e.g., E978m, Catalog #35-6200); OX40 (e.g., ABM193); OY-TESI; p53; p53mutant; PAP; PAX3 (e.g., GT1210, ThermoFisher Catalog #MA5-31583); PAX5; PDGFR-B (e.g., rinucumab); PDPN (e.g., ThermoFisher Catalog #14-5381-82); PLAVl; PMSA; polysialic acid (see, e.g., Watzlawik et al. J Nat Sci. 2015; 1(8):e141); PRI; PROMI (e.g., Catalog #14-1331-82); PSA (e.g., ThermoFisher Catalog #PA1-38514; Daniels-Wells et al. BMC Cancer 2013; 13:195); PSCA (e.g., AGS-1C4D4); PSMA (e.g., BAY 2315497); PTK7 (e.g., cofetuzumab); PVRIG; Ras mutant (e.g., Shin et al. Sci Adv. 2020; 6(3):eaay2174); RET (e.g., WO2020210551); RGS5 (e.g., TF-TA503075); RhoC (e.g., ThermoFisher Catalog PA5-77866); ROR1 (e.g., cirmtuzumab); ROR2 (e.g., BA3021); ROSI (e.g., WO 2019107671); Sarcoma translocation breakpoints; SART3 (e.g., TF 18025-1-AP); Sialyl-Thomsen-nouveau-antigen (e.g., Eavarone et al. PLoS One. 2018; 13(7): e0201314); Siglecs 1-16 (see, e.g., Angata et al. Trends Pharmacol Sci. 2015; 36(10): 645-660); SIRPa (e.g., Catalog #17-1729-42); SIRPg (e.g., PA5-104381); SIT1 (e.g., PA5-53825); SLAMF7 (e.g., elotuzumab); SLCIOA2 (e.g., ThermoFisher Catalog #PA5-18990); SLC12A2 (e.g., ThermoFisher Catalog #13884-1-AP); SLC17A2 (e.g., ThermoFisher Catalog #PA5-106752); SLC38A1 (e.g., ThermoFisher Catalog #12039-1-AP); SLC39A5 (e.g., ThermoFisher Catalog #MA5-27260); SLC39A6 (e.g., ladiratuzumab); SLC44A4 (e.g., ASG-5ME); SLC6A15 (e.g., ThermoFisher Catalog #PA5-52586); SLC6A6 (e.g., ThermoFisher Catalog #PA5-53431); SLC7A11 (e.g., ThermoFisher Catalog #PA1-16893); SLC7A5; sLe; SLITRK6 (e.g., sirtratumab); Sperm protein 17 (e.g., BS-5754R); SSX2 (e.g., ThermoFisher Catalog #MA5-24971); survivin (e.g., PA1-16836); TACSTD2 (e.g., PA5-47074); TAG-72 (e.g., MA1-25956); tenascin; TF (e.g., tisotumab); Tie3; TLR2/4/1 (e.g., tomaralimab); TM4SF5 (e.g., 18239-1-AP); TMEM132A (e.g., Catalog #PA5-62524); TMEM40 (e.g., PA5-60636); TMPRSS11D (e.g., PA5-30927); Tn; TNFRSF12 (e.g., BAY-356); TRAIL (e.g., Catalog #12-9927-42); TRAIL1; TRP-2 (e.g., PA5-52736); ULBP1/2/3/4/5/6 (e.g., PA5-82302); uPAR (e.g., ATN-658); UPK1B (e.g., ThermoFisher Catalog #PA5-56863); UPK2 (e.g., ThermoFisher Catalog #PA5-60318); UPK3B (e.g., ThermoFisher Catalog #PA5-52696); VEGF (e.g., GNR-011); VEGFR2 (e.g., gentuximab); VSIR (e.g., ThermoFisher Catalog #PA5-52493); WT1 (e.g., ThermoFisher Catalog #MA5-32215); and XAGE1 (e.g., ThermoFisher Catalog #PA5-46413).

Non-limiting examples of target antigens include Ax1 (e.g., BA3011; tilvestamab); B7-1 (e.g., galiximab); B7-2 (e.g., Catalog #12-0862-82); B7-DC (e.g., Catalog #PA5-20344); B7-H3 (e.g., enoblituzumab, omburtamab, MGD009, MGC018, DS-7300); B7-H4 (e.g., Catalog #14-5949-82); B7-H6 (e.g., Catalog #12-6526-42); B7-H7; BAFF-R (e.g., Catalog #14-9117-82); BCMA; C5 complement (e.g., BCD-148; CAN106); CCR4 (e.g., AT008; mogamulizumab-kpkc); CCR8 (e.g., JTX-1811); CD112 (see, e.g., U.S. Publication No. 20100008928); CD115 (e.g., axatilimab; cabiralizumab; emactuzumab); CD123 (e.g., BAY-943; CSL360); CD137 (e.g., ADG106; CTX-471); CD155 (e.g., U.S. Publication No. 2018/0251548); CD163 (e.g., TBI 304H); CD19 (e.g., ALLO-501); CD2 (e.g., BTI-322; siplizumab); CD20 (e.g., divozilimab; ibritumomab); CD24 (see, e.g., U.S. Pat. No. 8,614,301); CD244 (e.g., R&D AF1039); CD247 (e.g., AFM15); CD25 (e.g., basiliximab); CD27 (e.g., varlilumab); CD274 (e.g., adebrelimab; atezolizumab; garivulimab); CD278 (e.g., feladilimab; vopratelimab); CD28 (e.g., REGN5668); CD3 (e.g., otelixizumab; visilizumab); CD30 (e.g., iratumumab); CD30L (see, e.g., U.S. Pat. No. 9,926,373); CD32 (e.g., mAb 2B6); CD33 (e.g., lintuzumab; BI 836858; AMG 673); CD352 (e.g., SGN-CD352A); CD37 (e.g., lilotomab; GEN3009); CD38 (e.g., felzartamab; AMG 424); CD3D; CD3E (e.g., foralumab; teplizumab); CD3G; CD40 (e.g., dacetuzumab; lucatumumab); CD44 (e.g., RG7356); CD45 (e.g., apamistamab); CD47 (e.g., letaplimab; magrolimab); CD48 (e.g., SGN-CD48A); CD5 (e.g., MAT 304; zolimomab aritox); CD51; CD70 (e.g., cusatuzumab); CD74 (e.g., milatuzumab); CD79A (see, e.g., International Publication No. WO 2020252110); CD83 (e.g., CBT004); CD97; CD262 (e.g., tigatuzumab); CLEC12A (e.g., tepoditamab); CTLA4 (e.g., ipilimumab); CXCR4 (e.g., ulocuplumab); DCIR; DCSIGN (see, e.g., International Publication No. WO 2018134389); Dectin1 (see, e.g., U.S. Pat. No. 9,045,542); Dectin2 (e.g., ThermoFisher Catalog #MA5-16250); DR4 (e.g., mapatumumab); endosialin (e.g., ontuxizumab); FasL; FLT3 (e.g., 4G8SDIEM); GITR (e.g., ragifilimab); HAVCR2; HER2; HER3; HLA-DR; HLA-E; HLA-F; HLA-G (e.g., TTX-080); ICAM1; IDO1; IFNAR1 (e.g., faralimomab); IFNAR2; IGF-1R; ILIRAP (e.g., nidanilimab); IL-21R (e.g., PF-05230900); IL-5R (e.g., benralizumab); Integrin αvβ6; LAG-3 (e.g., encelimab); LAMP1; LAYN; LCK; LILRB2; LILRB4; MerTk (e.g., DS5MMER, Catalog #12-5751-82); Mesothelin; MICA (e.g., 1E2C8, Catalog #66384-1-IG); MICB (e.g., Catalog #MA5-29422); MICA; Mincle (e.g., OTI2A8, Catalog #TA505101); MRC1 (e.g., ThermoFisher Catalog #12-2061-82); MUC1; Muc16; NcaPi2B; Nectin-4; OX40 (e.g., ABM193); PD-1 (e.g., balstilimab; budigalimab; geptanolimab); PD-L1; Prolactin receptor; PTK7; PVRIG; ROR-1; Sialyl-Thomsen-nouveau-antigen (e.g., Eavarone et al. PLoS One, 2018; 13(7): e0201314); Siglecs 1-16 (see, e.g., Angata et al. Trends Pharmacol Sci. 2015; 36(10): 645-660); SIRPa (e.g., Catalog #17-1729-42); SIRPg (e.g., PA5-104381); SIT1 (e.g., PA5-53825); SLAMF7 (e.g., elotuzumab); SLTRK6; STEAP1; TIGIT (e.g., etigilimab); TLR2/4/1 (e.g., tomaralimab); Trem2 (e.g., PY314); TROP2; Tyrol; ULBP1/2/3/4/5/6 (e.g., PA5-82302); uPAR (e.g., ATN-658); VSIR (e.g., ThermoFisher Catalog #PA5-52493); ZIP6 (Anti-Integrin αvβ6).

(i) Heavy Chain and Light Chain Variable Regions

In some cases, an antibody target is selected from the group consisting of ADAM9, ASCT2, Ax1, B7-H3, B7H4, BCMA, BCMA, C4.4a, CanAg, CD123, CD138, CD142, CD166, CD19, CD20, CD228, CD25, CD30, CD33, CD352, CD38, CD48, CD56, CD59, CD70, CD74, CD79b, CDCP1, CEACAM5, Claudin-18.2, c-Met, gpNMB, CS1, DLL-3, DPEP-3, EGFR, EpCAM, EphA2, FGFR2, FRa, GCC, gpA33, GPC3, Integrin αvβ6, h2A2, H2G12/STn, HER2, HER3, ZIP6, IGF-1R, IL1Rap, ITGav/CD51, Mesothelin, MICA, MUC-1, Muc16, NaPi2B, Nectin-4, PD-L1, Prolactin receptor, PTK7, ROR-1, SLAMF7, SLTRK6, STEAP1, TIGIT, and TROP2.

In some cases, an antibody of the present disclosure comprises a heavy chain variable region having at least 80% sequence identity to a first sequence and a light chain variable region having at least 80% sequence identity to a second sequence, and wherein: the first sequence is SEQ ID NO: 19 and the second sequence is SEQ ID NO: 20; the first sequence is SEQ ID NO: 44 and the second sequence is SEQ ID NO: 45; the first sequence is SEQ ID NO: 55 and the second sequence is SEQ ID NO: 56; the first sequence is SEQ ID NO: 69 and the second sequence is SEQ ID NO: 70; the first sequence is SEQ ID NO: 83 and the second sequence is SEQ ID NO: 84; the first sequence is SEQ ID NO: 97 and the second sequence is SEQ ID NO: 98; the first sequence is SEQ ID NO: 105 and the second sequence is SEQ ID NO: 106; the first sequence is SEQ ID NO:113 and the second sequence is SEQ ID NO: 114; the first sequence is SEQ ID NO: 121 and the second sequence is SEQ ID NO: 122; the first sequence is SEQ ID NO: 129 and the second sequence is SEQ ID NO: 130; the first sequence is SEQ ID NO: 137 and the second sequence is SEQ ID NO: 138; the first sequence is SEQ ID NO: 153 and the second sequence is SEQ ID NO: 154; the first sequence is SEQ ID NO: 161 and the second sequence is SEQ ID NO: 162; the first sequence is SEQ ID NO: 169 and the second sequence is SEQ ID NO: 170; the first sequence is SEQ ID NO: 177 and the second sequence is SEQ ID NO: 178; the first sequence is SEQ ID NO: 185 and the second sequence is SEQ ID NO: 186; the first sequence is SEQ ID NO: 193 and the second sequence is SEQ ID NO: 194; the first sequence is SEQ ID NO: 201 and the second sequence is SEQ ID NO: 202; the first sequence is SEQ ID NO: 209 and the second sequence is SEQ ID NO: 210; the first sequence is SEQ ID NO: 217 and the second sequence is SEQ ID NO: 218; the first sequence is SEQ ID NO: 225 and the second sequence is SEQ ID NO: 226; the first sequence is SEQ ID NO: 233 and the second sequence is SEQ ID NO: 234; the first sequence is SEQ ID NO: 241 and the second sequence is SEQ ID NO: 242; the first sequence is SEQ ID NO: 249 and the second sequence is SEQ ID NO: 250; the first sequence is SEQ ID NO: 297 and the second sequence is SEQ ID NO: 298; the first sequence is SEQ ID NO: 307 and the second sequence is SEQ ID NO: 308; the first sequence is SEQ ID NO: 315 and the second sequence is SEQ ID NO: 316; the first sequence is SEQ ID NO: 323 and the second sequence is SEQ ID NO: 324; the first sequence is SEQ ID NO: 331 and the second sequence is SEQ ID NO: 332; the first sequence is SEQ ID NO: 339 and the second sequence is SEQ ID NO: 340; the first sequence is SEQ ID NO: 347 and the second sequence is SEQ ID NO: 348; the first sequence is SEQ ID NO: 355 and the second sequence is SEQ ID NO: 356; the first sequence is SEQ ID NO: 363 and the second sequence is SEQ ID NO: 364; the first sequence is SEQ ID NO: 371 and the second sequence is SEQ ID NO: 372; the first sequence is SEQ ID NO: 379 and the second sequence is SEQ ID NO: 380; the first sequence is SEQ ID NO: 387 and the second sequence is SEQ ID NO: 388; the first sequence is SEQ ID NO: 395 and the second sequence is SEQ ID NO: 396; the first sequence is SEQ ID NO: 403 and the second sequence is SEQ ID NO: 404; the first sequence is SEQ ID NO: 411 and the second sequence is SEQ ID NO: 412; the first sequence is SEQ ID NO: 419 and the second sequence is SEQ ID NO: 420; the first sequence is SEQ ID NO: 427 and the second sequence is SEQ ID NO: 428; the first sequence is SEQ ID NO: 435 and the second sequence is SEQ ID NO: 436; the first sequence is SEQ ID NO: 443 and the second sequence is SEQ ID NO: 444; the first sequence is SEQ ID NO: 451 and the second sequence is SEQ ID NO: 452; the first sequence is SEQ ID NO: 459 and the second sequence is SEQ ID NO: 460; the first sequence is SEQ ID NO: 467 and the second sequence is SEQ ID NO: 468; the first sequence is SEQ ID NO: 475 and the second sequence is SEQ ID NO: 476; the first sequence is SEQ ID NO: 483 and the second sequence is SEQ ID NO: 484; the first sequence is SEQ ID NO: 491 and the second sequence is SEQ ID NO: 492; the first sequence is SEQ ID NO: 501 and the second sequence is SEQ ID NO: 502; the first sequence is SEQ ID NO: 509 and the second sequence is SEQ ID NO: 510; the first sequence is SEQ ID NO: 517 and the second sequence is SEQ ID NO: 518; the first sequence is SEQ ID NO: 525 and the second sequence is SEQ ID NO: 526; the first sequence is SEQ ID NO: 533 and the second sequence is SEQ ID NO: 534; the first sequence is SEQ ID NO: 541 and the second sequence is SEQ ID NO: 542; the first sequence is SEQ ID NO: 549 and the second sequence is SEQ ID NO: 550; the first sequence is SEQ ID NO: 557 and the second sequence is SEQ ID NO: 558; the first sequence is SEQ ID NO: 565 and the second sequence is SEQ ID NO: 566; the first sequence is SEQ ID NO: 574 and the second sequence is SEQ ID NO: 574; the first sequence is SEQ ID NO: 581 and the second sequence is SEQ ID NO: 582; the first sequence is SEQ ID NO: 589 and the second sequence is SEQ ID NO: 590; the first sequence is SEQ ID NO: 597 and the second sequence is SEQ ID NO: 598; the first sequence is SEQ ID NO: 605 and the second sequence is SEQ ID NO: 606; the first sequence is SEQ ID NO: 613 and the second sequence is SEQ ID NO: 614; the first sequence is SEQ ID NO: 621 and the second sequence is SEQ ID NO: 622; the first sequence is SEQ ID NO: 629 and the second sequence is SEQ ID NO: 630; the first sequence is SEQ ID NO: 637 and the second sequence is SEQ ID NO: 638; the first sequence is SEQ ID NO: 645 and the second sequence is SEQ ID NO: 646; the first sequence is SEQ ID NO: 653 and the second sequence is SEQ ID NO: 654; the first sequence is SEQ ID NO: 661 and the second sequence is SEQ ID NO: 662; the first sequence is SEQ ID NO: 669 and the second sequence is SEQ ID NO: 670; the first sequence is SEQ ID NO: 677 and the second sequence is SEQ ID NO: 678; the first sequence is SEQ ID NO: 685 and the second sequence is SEQ ID NO: 686; the first sequence is SEQ ID NO: 693 and the second sequence is SEQ ID NO: 694; the first sequence is SEQ ID NO: 701 and the second sequence is SEQ ID NO: 702; the first sequence is SEQ ID NO: 703 and the second sequence is SEQ ID NO: 704; the first sequence is SEQ ID NO: 711 and the second sequence is SEQ ID NO: 712; the first sequence is SEQ ID NO: 713 and the second sequence is SEQ ID NO: 714; the first sequence is SEQ ID NO: 715 and the second sequence is SEQ ID NO: 716; the first sequence is SEQ ID NO: 731 and the second sequence is SEQ ID NO: 732; the first sequence is SEQ ID NO: 739 and the second sequence is SEQ ID NO: 740; the first sequence is SEQ ID NO: 747 and the second sequence is SEQ ID NO: 748; the first sequence is SEQ ID NO: 755 and the second sequence is SEQ ID NO: 756; the first sequence is SEQ ID NO: 765 and the second sequence is SEQ ID NO: 766; the first sequence is SEQ ID NO: 773 and the second sequence is SEQ ID NO: 774; the first sequence is SEQ ID NO: 781 and the second sequence is SEQ ID NO: 782; the first sequence is SEQ ID NO: 789 and the second sequence is SEQ ID NO: 790; the first sequence is SEQ ID NO: 797 and the second sequence is SEQ ID NO: 798; the first sequence is SEQ ID NO: 805 and the second sequence is SEQ ID NO: 806; the first sequence is SEQ ID NO: 813 and the second sequence is SEQ ID NO: 814; the first sequence is SEQ ID NO: 821 and the second sequence is SEQ ID NO: 822; the first sequence is SEQ ID NO: 829 and the second sequence is SEQ ID NO: 830; the first sequence is SEQ ID NO: 837 and the second sequence is SEQ ID NO: 838; the first sequence is SEQ ID NO: 845 and the second sequence is SEQ ID NO: 846; the first sequence is SEQ ID NO: 853 and the second sequence is SEQ ID NO: 854; the first sequence is SEQ ID NO: 861 and the second sequence is SEQ ID NO: 862; the first sequence is SEQ ID NO: 869 and the second sequence is SEQ ID NO: 870; the first sequence is SEQ ID NO: 877 and the second sequence is SEQ ID NO: 878; the first sequence is SEQ ID NO: 885 and the second sequence is SEQ ID NO: 886; the first sequence is SEQ ID NO: 893 and the second sequence is SEQ ID NO: 894; the first sequence is SEQ ID NO: 900 and the second sequence is SEQ ID NO: 901; the first sequence is SEQ ID NO: 909 and the second sequence is SEQ ID NO: 910; the first sequence is SEQ ID NO: 917 and the second sequence is SEQ ID NO: 918; the first sequence is SEQ ID NO: 925 and the second sequence is SEQ ID NO: 926; the first sequence is SEQ ID NO: 933 and the second sequence is SEQ ID NO: 934; the first sequence is SEQ ID NO: 941 and the second sequence is SEQ ID NO: 942; the first sequence is SEQ ID NO: 943 and the second sequence is SEQ ID NO: 944; the first sequence is SEQ ID NO: 951 and the second sequence is SEQ ID NO: 952; the first sequence is SEQ ID NO: 959 and the second sequence is SEQ ID NO: 960; the first sequence is SEQ ID NO: 967 and the second sequence is SEQ ID NO: 968; the first sequence is SEQ ID NO: 975 and the second sequence is SEQ ID NO: 976; the first sequence is SEQ ID NO: 983 and the second sequence is SEQ ID NO: 984; the first sequence is SEQ ID NO: 991 and the second sequence is SEQ ID NO: 992; the first sequence is SEQ ID NO: 993 and the second sequence is SEQ ID NO: 994; the first sequence is SEQ ID NO: 995 and the second sequence is SEQ ID NO: 996; the first sequence is SEQ ID NO: 1003 and the second sequence is SEQ ID NO: 1004; the first sequence is SEQ ID NO: 1011 and the second sequence is SEQ ID NO: 1012; the first sequence is SEQ ID NO: 1019 and the second sequence is SEQ ID NO: 1020; the first sequence is SEQ ID NO: 1027 and the second sequence is SEQ ID NO: 1028; the first sequence is SEQ ID NO: 1035 and 1036; or the first sequence is SEQ ID NO: 1043 and the second sequence is SEQ ID NO: 1044.

In some cases, an antibody of the present disclosure targets an immune checkpoint selected from the group consisting of PDL1, B7H4, B7H3, and TIGIT. For example, in some cases, the antibody comprises a heavy chain variable region having at least 80% sequence identity to a first sequence and a light chain variable region having at least 80% sequence identity to a second sequence, wherein: the first sequence is SEQ ID NO: 19 and the second sequence is SEQ ID NO: 20; the first sequence is SEQ ID NO: 83 and the second sequence is SEQ ID NO: 84; the first sequence is SEQ ID NO: 97 and the second sequence is SEQ ID NO: 98; the first sequence is SEQ ID NO: 105 and the second sequence is SEQ ID NO: 106; the first sequence is SEQ ID NO:113 and the second sequence is SEQ ID NO: 114; the first sequence is SEQ ID NO: 121 and the second sequence is SEQ ID NO: 122; the first sequence is SEQ ID NO: 129 and the second sequence is SEQ ID NO: 130; the first sequence is SEQ ID NO: 137 and the second sequence is SEQ ID NO: 138; the first sequence is SEQ ID NO: 153 and the second sequence is SEQ ID NO: 154; the first sequence is SEQ ID NO: 161 and the second sequence is SEQ ID NO: 162; the first sequence is SEQ ID NO: 169 and the second sequence is SEQ ID NO: 170; the first sequence is SEQ ID NO: 177 and the second sequence is SEQ ID NO: 178; the first sequence is SEQ ID NO: 185 and the second sequence is SEQ ID NO: 186; the first sequence is SEQ ID NO: 193 and the second sequence is SEQ ID NO: 194; the first sequence is SEQ ID NO: 201 and the second sequence is SEQ ID NO: 202; the first sequence is SEQ ID NO: 209 and the second sequence is SEQ ID NO: 210; the first sequence is SEQ ID NO: 217 and the second sequence is SEQ ID NO: 218; the first sequence is SEQ ID NO: 225 and the second sequence is SEQ ID NO: 226; the first sequence is SEQ ID NO: 233 and the second sequence is SEQ ID NO: 234; the first sequence is SEQ ID NO: 241 and the second sequence is SEQ ID NO: 242; the first sequence is SEQ ID NO: 249 and the second sequence is SEQ ID NO: 250; the first sequence is SEQ ID NO: 629 and the second sequence is SEQ ID NO: 630; the first sequence is SEQ ID NO: 637 and the second sequence is SEQ ID NO: 638; the first sequence is SEQ ID NO: 645 and the second sequence is SEQ ID NO: 646; the first sequence is SEQ ID NO: 653 and the second sequence is SEQ ID NO: 654; the first sequence is SEQ ID NO: 661 and the second sequence is SEQ ID NO: 662; the first sequence is SEQ ID NO: 669 and the second sequence is SEQ ID NO: 670; the first sequence is SEQ ID NO: 677 and the second sequence is SEQ ID NO: 678; the first sequence is SEQ ID NO: 685 and the second sequence is SEQ ID NO: 686; the first sequence is SEQ ID NO: 693 and the second sequence is SEQ ID NO: 694; the first sequence is SEQ ID NO: 701 and the second sequence is SEQ ID NO: 702; the first sequence is SEQ ID NO: 703 and the second sequence is SEQ ID NO: 704; the first sequence is SEQ ID NO: 711 and the second sequence is SEQ ID NO: 712; the first sequence is SEQ ID NO: 713 and the second sequence is SEQ ID NO: 714; the first sequence is SEQ ID NO: 715 and the second sequence is SEQ ID NO: 716; or the first sequence is SEQ ID NO: 1027 and the second sequence is SEQ ID NO: 1028.

In some cases, an antibody of the present disclosure comprises a heavy chain variable region having at least 80% sequence identity to a first sequence and a light chain variable region having at least 80% sequence identity to a second sequence, wherein: the first sequence is SEQ ID NO: 19 and the second sequence is SEQ ID NO: 20; the first sequence is SEQ ID NO: 83 and the second sequence is SEQ ID NO: 84; the first sequence is SEQ ID NO: 97 and the second sequence is SEQ ID NO: 98; the first sequence is SEQ ID NO: 105 and the second sequence is SEQ ID NO: 106; the first sequence is SEQ ID NO: 113 and the second sequence is SEQ ID NO: 114; the first sequence is SEQ ID NO: 121 and the second sequence is SEQ ID NO: 122; the first sequence is SEQ ID NO: 129 and the second sequence is SEQ ID NO: 130; the first sequence is SEQ ID NO: 137 and the second sequence is SEQ ID NO: 138; the first sequence is SEQ ID NO: 153 and the second sequence is SEQ ID NO: 154; the first sequence is SEQ ID NO: 161 and the second sequence is SEQ ID NO: 162; the first sequence is SEQ ID NO: 169 and the second sequence is SEQ ID NO: 170; the first sequence is SEQ ID NO: 177 and the second sequence is SEQ ID NO: 178; the first sequence is SEQ ID NO: 185 and the second sequence is SEQ ID NO: 186; the first sequence is SEQ ID NO: 193 and the second sequence is SEQ ID NO: 194; the first sequence is SEQ ID NO: 201 and the second sequence is SEQ ID NO: 202; the first sequence is SEQ ID NO: 209 and the second sequence is SEQ ID NO: 210; the first sequence is SEQ ID NO: 217 and the second sequence is SEQ ID NO: 218; the first sequence is SEQ ID NO: 225 and the second sequence is SEQ ID NO: 226; the first sequence is SEQ ID NO: 233 and the second sequence is SEQ ID NO: 234; the first sequence is SEQ ID NO: 241 and the second sequence is SEQ ID NO: 242; the first sequence is SEQ ID NO: 249 and the second sequence is SEQ ID NO: 250; or the first sequence is SEQ ID NO: 1027 and the second sequence is SEQ ID NO: 1028. In some cases, the first sequence is SEQ ID NO: 19 and the second sequence is SEQ ID NO: 20.

In some cases, the heavy chain variable region has at least 85% sequence identity to the first sequence and the light chain variable region has at least 85% sequence identity to the second sequence. In some cases, the heavy chain variable region has at least 90% sequence identity to the first sequence and the light chain variable region has at least 90% sequence identity to the second sequence. In some cases, the heavy chain variable region has at least 95% sequence identity to the first sequence and the light chain variable region has at least 95% sequence identity to the second sequence. In some cases, the heavy chain variable region has at least 98% sequence identity to the first sequence and the light chain variable region has at least 98% sequence identity to the second sequence. In some cases, the heavy chain variable region has at least 99% sequence identity to the first sequence and the light chain variable region has at least 99% sequence identity to the second sequence. In some cases, the heavy chain variable region comprises the first sequence and the light chain variable region comprises the second sequence.

(ii) Heavy and Light Chains

In some cases, an antibody of the present disclosure comprises a heavy chain having at least 80% sequence identity to a first sequence and a light chain having at least 80% sequence identity to a second sequence, wherein: the first sequence is SEQ ID NO: 1 and the second sequence is SEQ ID NO: 5; the first sequence is SEQ ID NO: 2 and the second sequence is SEQ ID NO: 5; the first sequence is SEQ ID NO: 3 and the second sequence is SEQ ID NO: 5; the first sequence is SEQ ID NO: 4 and the second sequence is SEQ ID NO: 5; the first sequence is SEQ ID NO: 6 and the second sequence is SEQ ID NO: 10; the first sequence is SEQ ID NO: 7 and the second sequence is SEQ ID NO: 10; the first sequence is SEQ ID NO: 8 and the second sequence is SEQ ID NO: 10; the first sequence is SEQ ID NO: 9 and the second sequence is SEQ ID NO: 10; the first sequence is SEQ ID NO: 46 and the second sequence is SEQ ID NO: 48; the first sequence is SEQ ID NO: 47 and the second sequence is SEQ ID NO: 48; the first sequence is SEQ ID NO: 57 and the second sequence is SEQ ID NO: 59; the first sequence is SEQ ID NO: 58 and the second sequence is SEQ ID NO: 59; the first sequence is SEQ ID NO: 60 and the second sequence is SEQ ID NO: 62; the first sequence is SEQ ID NO: 61 and the second sequence is SEQ ID NO: 62; the first sequence is SEQ ID NO: 71 and the second sequence is SEQ ID NO: 73; the first sequence is SEQ ID NO: 72 and the second sequence is SEQ ID NO: 73; the first sequence is SEQ ID NO: 74 and the second sequence is SEQ ID NO: 76; the first sequence is SEQ ID NO: 75 and the second sequence is SEQ ID NO: 76; the first sequence is SEQ ID NO: 85 and the second sequence is SEQ ID NO: 87; the first sequence is SEQ ID NO: 86 and the second sequence is SEQ ID NO: 87; the first sequence is SEQ ID NO: 88 and the second sequence is SEQ ID NO: 90; the first sequence is SEQ ID NO: 89 and the second sequence is SEQ ID NO: 90; the first sequence is SEQ ID NO: 251 and the second sequence is SEQ ID NO: 252; the first sequence is SEQ ID NO: 253 and the second sequence is SEQ ID NO: 254; the first sequence is SEQ ID NO: 255 and the second sequence is SEQ ID NO: 256; the first sequence is SEQ ID NO: 257 and the second sequence is SEQ ID NO: 258; the first sequence is SEQ ID NO: 259 and the second sequence is SEQ ID NO: 260; the first sequence is SEQ ID NO: 261 and the second sequence is SEQ ID NO: 262; the first sequence is SEQ ID NO: 263 and the second sequence is SEQ ID NO: 264; the first sequence is SEQ ID NO: 265 and the second sequence is SEQ ID NO: 266; the first sequence is SEQ ID NO: 267 and the second sequence is SEQ ID NO: 268; the first sequence is SEQ ID NO: 269 and the second sequence is SEQ ID NO: 270; the first sequence is SEQ ID NO: 271 and the second sequence is SEQ ID NO: 272; the first sequence is SEQ ID NO: 273 and the second sequence is SEQ ID NO: 274; the first sequence is SEQ ID NO: 275 and the second sequence is SEQ ID NO: 276; the first sequence is SEQ ID NO: 277 and the second sequence is SEQ ID NO: 278; the first sequence is SEQ ID NO: 279 and the second sequence is SEQ ID NO: 280; the first sequence is SEQ ID NO: 281 and the second sequence is SEQ ID NO: 282; the first sequence is SEQ ID NO: 283 and the second sequence is SEQ ID NO: 284; the first sequence is SEQ ID NO: 285 and the second sequence is SEQ ID NO: 286; the first sequence is SEQ ID NO: 287 and the second sequence is SEQ ID NO: 288; the first sequence is SEQ ID NO: 289 and the second sequence is SEQ ID NO: 290; the first sequence is SEQ ID NO: 299 and the second sequence is SEQ ID NO: 300; the first sequence is SEQ ID NO: 493 and the second sequence is SEQ ID NO: 494; the first sequence is SEQ ID NO: 717 and the second sequence is SEQ ID NO: 718; the first sequence is SEQ ID NO: 719 and the second sequence is SEQ ID NO: 720; the first sequence is SEQ ID NO: 721 and the second sequence is SEQ ID NO: 722; the first sequence is SEQ ID NO: 723 and the second sequence is SEQ ID NO: 724; or the first sequence is SEQ ID NO: 757 and the second sequence is SEQ ID NO: 758.

In some cases, an antibody of the present disclosure comprises a heavy chain having at least 80% sequence identity to a first sequence and a light chain having at least 80% sequence identity to a second sequence, wherein: the first sequence is SEQ ID NO: 1 and the second sequence is SEQ ID NO: 5; the first sequence is SEQ ID NO: 2 and the second sequence is SEQ ID NO: 5; the first sequence is SEQ ID NO: 3 and the second sequence is SEQ ID NO: 5; the first sequence is SEQ ID NO: 4 and the second sequence is SEQ ID NO: 5; the first sequence is SEQ ID NO: 6 and the second sequence is SEQ ID NO: 10; the first sequence is SEQ ID NO: 7 and the second sequence is SEQ ID NO: 10; the first sequence is SEQ ID NO: 8 and the second sequence is SEQ ID NO: 10; the first sequence is SEQ ID NO: 9 and the second sequence is SEQ ID NO: 10; the first sequence is SEQ ID NO: 85 and the second sequence is SEQ ID NO: 87; the first sequence is SEQ ID NO: 86 and the second sequence is SEQ ID NO: 87; the first sequence is SEQ ID NO: 88 and the second sequence is SEQ ID NO: 90; the first sequence is SEQ ID NO: 89 and the second sequence is SEQ ID NO: 90; the first sequence is SEQ ID NO: 251 and the second sequence is SEQ ID NO: 252; the first sequence is SEQ ID NO: 253 and the second sequence is SEQ ID NO: 254; the first sequence is SEQ ID NO: 255 and the second sequence is SEQ ID NO: 256; the first sequence is SEQ ID NO: 257 and the second sequence is SEQ ID NO: 258; the first sequence is SEQ ID NO: 259 and the second sequence is SEQ ID NO: 260; the first sequence is SEQ ID NO: 261 and the second sequence is SEQ ID NO: 262; the first sequence is SEQ ID NO: 263 and the second sequence is SEQ ID NO: 264; the first sequence is SEQ ID NO: 265 and the second sequence is SEQ ID NO: 266; the first sequence is SEQ ID NO: 267 and the second sequence is SEQ ID NO: 268; the first sequence is SEQ ID NO: 269 and the second sequence is SEQ ID NO: 270; the first sequence is SEQ ID NO: 271 and the second sequence is SEQ ID NO: 272; the first sequence is SEQ ID NO: 273 and the second sequence is SEQ ID NO: 274; the first sequence is SEQ ID NO: 275 and the second sequence is SEQ ID NO: 276; the first sequence is SEQ ID NO: 277 and the second sequence is SEQ ID NO: 278; the first sequence is SEQ ID NO: 279 and the second sequence is SEQ ID NO: 280; the first sequence is SEQ ID NO: 281 and the second sequence is SEQ ID NO: 282; the first sequence is SEQ ID NO: 283 and the second sequence is SEQ ID NO: 284; the first sequence is SEQ ID NO: 285 and the second sequence is SEQ ID NO: 286; the first sequence is SEQ ID NO: 287 and the second sequence is SEQ ID NO: 288; the first sequence is SEQ ID NO: 289 and the second sequence is SEQ ID NO: 290; the first sequence is SEQ ID NO: 717 and the second sequence is SEQ ID NO: 718; the first sequence is SEQ ID NO: 719 and the second sequence is SEQ ID NO: 720; the first sequence is SEQ ID NO: 721 and the second sequence is SEQ ID NO: 722; or the first sequence is SEQ ID NO: 723 and the second sequence is SEQ ID NO: 724.

In some cases, an antibody of the present disclosure comprises a heavy chain having at least 80% sequence identity to a first sequence and a light chain having at least 80% sequence identity to a second sequence, wherein: the first sequence is SEQ ID NO: 1 and the second sequence is SEQ ID NO: 5; the first sequence is SEQ ID NO: 2 and the second sequence is SEQ ID NO: 5; the first sequence is SEQ ID NO: 3 and the second sequence is SEQ ID NO: 5; the first sequence is SEQ ID NO: 4 and the second sequence is SEQ ID NO: 5; the first sequence is SEQ ID NO: 6 and the second sequence is SEQ ID NO: 10; the first sequence is SEQ ID NO: 7 and the second sequence is SEQ ID NO: 10; the first sequence is SEQ ID NO: 8 and the second sequence is SEQ ID NO: 10; the first sequence is SEQ ID NO: 9 and the second sequence is SEQ ID NO: 10; the first sequence is SEQ ID NO: 85 and the second sequence is SEQ ID NO: 87; the first sequence is SEQ ID NO: 86 and the second sequence is SEQ ID NO: 87; the first sequence is SEQ ID NO: 88 and the second sequence is SEQ ID NO: 90; the first sequence is SEQ ID NO: 89 and the second sequence is SEQ ID NO: 90; the first sequence is SEQ ID NO: 251 and the second sequence is SEQ ID NO: 252; the first sequence is SEQ ID NO: 253 and the second sequence is SEQ ID NO: 254; the first sequence is SEQ ID NO: 255 and the second sequence is SEQ ID NO: 256; the first sequence is SEQ ID NO: 257 and the second sequence is SEQ ID NO: 258; the first sequence is SEQ ID NO: 259 and the second sequence is SEQ ID NO: 260; the first sequence is SEQ ID NO: 261 and the second sequence is SEQ ID NO: 262; the first sequence is SEQ ID NO: 263 and the second sequence is SEQ ID NO: 264; the first sequence is SEQ ID NO: 265 and the second sequence is SEQ ID NO: 266; the first sequence is SEQ ID NO: 267 and the second sequence is SEQ ID NO: 268; the first sequence is SEQ ID NO: 269 and the second sequence is SEQ ID NO: 270; the first sequence is SEQ ID NO: 271 and the second sequence is SEQ ID NO: 272; the first sequence is SEQ ID NO: 273 and the second sequence is SEQ ID NO: 274; the first sequence is SEQ ID NO: 275 and the second sequence is SEQ ID NO: 276; the first sequence is SEQ ID NO: 277 and the second sequence is SEQ ID NO: 278; the first sequence is SEQ ID NO: 279 and the second sequence is SEQ ID NO: 280; the first sequence is SEQ ID NO: 281 and the second sequence is SEQ ID NO: 282; the first sequence is SEQ ID NO: 283 and the second sequence is SEQ ID NO: 284; the first sequence is SEQ ID NO: 285 and the second sequence is SEQ ID NO: 286; the first sequence is SEQ ID NO: 287 and the second sequence is SEQ ID NO: 288; or the first sequence is SEQ ID NO: 289 and the second sequence is SEQ ID NO: 290.

In some cases, an antibody of the present disclosure comprises a heavy chain having at least 80% sequence identity to a first sequence and a light chain having at least 80% sequence identity to a second sequence, wherein: the first sequence is SEQ ID NO: 1 and the second sequence is SEQ ID NO: 5; the first sequence is SEQ ID NO: 2 and the second sequence is SEQ ID NO: 5; the first sequence is SEQ ID NO: 3 and the second sequence is SEQ ID NO: 5; the first sequence is SEQ ID NO: 4 and the second sequence is SEQ ID NO: 5; the first sequence is SEQ ID NO: 6 and the second sequence is SEQ ID NO: 10; the first sequence is SEQ ID NO: 7 and the second sequence is SEQ ID NO: 10; the first sequence is SEQ ID NO: 8 and the second sequence is SEQ ID NO: 10; or the first sequence is SEQ ID NO: 9 and the second sequence is SEQ ID NO: 10.

In some cases, an antibody of the present disclosure comprises a heavy chain having at least 80% sequence identity to a first sequence and a light chain having at least 80% sequence identity to a second sequence, wherein: the first sequence is SEQ ID NO: 3 or SEQ ID NO: 4 and the second sequence is SEQ ID NO: 5.

In some cases, the heavy chain has at least 85% sequence identity to the first sequence and the light chain has at least 85% sequence identity to the second sequence. In some cases, the heavy chain has at least 90% sequence identity to the first sequence and the light chain has at least 90% sequence identity to the second sequence. In some cases, the heavy chain has at least 95% sequence identity to the first sequence and the light chain has at least 95% sequence identity to the second sequence. In some cases, the heavy chain has at least 98% sequence identity to the first sequence and the light chain has at least 98% sequence identity to the second sequence. In some cases, the heavy chain has at least 99% sequence identity to the first sequence and the light chain has at least 99% sequence identity to the second sequence. In some cases, the heavy chain comprises the first sequence and the light chain comprises the second sequence.

(iii) Complementarity-Determining Regions

In some cases, an antibody of the present disclosure comprises CDR-H1, CDR-H2, CDR-H3, CDR-L1, CDR-L2, and CDR-L3 comprising at least 80% sequence identity to: SEQ ID NO: 13, 14, 15, 16, 17, and 18, respectively (i.e., CDR-H1 has at least 80% sequence identity to SEQ ID NO: 13, CDR-H2 has at least 80% sequence identity to SEQ ID NO: 14, CDR-H3 has at least 80% sequence identity to SEQ ID NO: 15, CDR-L1 has at least 80% sequence identity to SEQ ID NO: 16, CDR-L2 has at least 80% sequence identity to SEQ ID NO: 17, and CDR-L3 has at least 80% sequence identity to SEQ ID NO: 18); SEQ ID NO: 21, 22, 23, 24, 25, and 26, respectively; SEQ ID NO: 38, 39, 40, 41, 42, and 43, respectively; SEQ ID NO: 49, 50, 51, 52, 53, and 54, respectively; SEQ ID NO: 63, 64, 65, 66, 67, and 68, respectively; SEQ ID NO: 77, 78, 79, 80, 81, and 82, respectively; SEQ ID NO: 91, 92, 93, 94, 95, and 96, respectively; SEQ ID NO: 99, 100, 101, 102, 103, and 104, respectively; SEQ ID NO: 107, 108, 109, 110, 111, and 112, respectively; SEQ ID NO: 115, 116, 117, 118, 119, and 120, respectively; SEQ ID NO: 123, 124, 125, 126, 127, and 128, respectively; SEQ ID NO: 131, 132, 133, 134, 135, and 136, respectively; SEQ ID NO: 139, 140, 141, 142, 143, and 144, respectively; SEQ ID NO: 147, 148, 149, 150, 151, and 152, respectively; SEQ ID NO: 155, 156, 157, 158, 159, and 160, respectively; SEQ ID NO: 163, 164, 165, 166, 167, and 168, respectively; SEQ ID NO: 171, 172, 173, 174, 175, and 176, respectively; SEQ ID NO: 179, 180, 181, 182, 183, and 184, respectively; SEQ ID NO: 187, 188, 189, 190, 191, and 192, respectively; SEQ ID NO: 195, 196, 197, 198, 199, and 200, respectively; SEQ ID NO: 203, 204, 205, 206, 207 and 208, respectively; SEQ ID NO: 211, 212, 213, 214, 215, and 216, respectively; SEQ ID NO: 219, 220, 221, 222, 223, and 224, respectively; SEQ ID NO: 227, 228, 229, 230, 231, and 232, respectively; SEQ ID NO: 235, 236, 237, 238, 239, and 240, respectively; SEQ ID NO: 243, 244, 245, 246, 247, and 248, respectively; SEQ ID NO: 291, 292, 293, 294, 295, and 296, respectively; SEQ ID NO: 301, 302, 303, 304, 305, and 306, respectively; SEQ ID NO: 309, 310, 311, 312, 313, and 314, respectively; SEQ ID NO: 317, 318, 319, 320, 321, and 322, respectively; SEQ ID NO: 325, 326, 327, 328, 329, and 330, respectively; SEQ ID NO: 333, 334, 335, 336, 337, and 338, respectively; SEQ ID NO: 341, 342, 343, 344, 345, and 346, respectively; SEQ ID NO: 349, 350, 351, 352, 353, and 354, respectively; SEQ ID NO: 357, 358, 359, 360, 361, and 362, respectively; SEQ ID NO: 365, 366, 367, 368, 369, and 370, respectively; SEQ ID NO: 373, 374, 375, 376, 377, and 378, respectively; SEQ ID NO: 381, 382, 383, 384, 385, and 386, respectively; SEQ ID NO: 389, 390, 391, 392, 393, and 394, respectively; SEQ ID NO: 397, 398, 399, 400, 401, and 402, respectively; SEQ ID NO: 405, 406, 407, 408, 409, and 410, respectively; SEQ ID NO: 413, 414, 415, 416, 417, and 418, respectively; SEQ ID NO: 421, 422, 423, 424, 425, and 426, respectively; SEQ ID NO: 429, 430, 431, 432, 433, and 434, respectively; SEQ ID NO: 437, 438, 439, 440, 441, and 442, respectively; SEQ ID NO: 445, 446, 447, 448, 449, and 450, respectively; SEQ ID NO: 453, 454, 455, 456, 457, and 458, respectively; SEQ ID NO: 461, 462, 463, 464, 465, and 466, respectively; SEQ ID NO: 469, 470, 471, 472, 473, and 474, respectively; SEQ ID NO: 477, 478, 479, 480, 481, and 482, respectively; SEQ ID NO: 485, 486, 487, 488, 489, and 490, respectively; SEQ ID NO: 495, 496, 497, 498, 499, and 500, respectively; SEQ ID NO: 503, 504, 505, 506, 507, and 508, respectively; SEQ ID NO: 511, 512, 513, 514, 515, and 516, respectively; SEQ ID NO: 519, 520, 521, 522, 523, and 524, respectively; SEQ ID NO: 527, 528, 529, 530, 531, and 532, respectively; SEQ ID NO: 535, 536, 537, 538, 539, and 540, respectively; SEQ ID NO: 543, 544, 545, 546, 547, and 548, respectively; SEQ ID NO: 551, 552, 553, 554, 555, and 556, respectively; SEQ ID NO: 559, 560, 561, 562, 563, and 564, respectively; SEQ ID NO: 567, 568, 569, 570, 571, and 572, respectively; SEQ ID NO: 575, 576, 577, 578, 579, and 580, respectively; SEQ ID NO: 583, 584, 585, 586, 587, and 588, respectively; SEQ ID NO: 591, 592, 593, 594, 595, and 596, respectively; SEQ ID NO: 599, 600, 601, 602, 603, and 604, respectively; SEQ ID NO: 607, 608, 609, 610, 611, and 612, respectively; SEQ ID NO: 615, 616, 617, 618, 619, and 620, respectively; SEQ ID NO: 623, 624, 625, 626, 627, and 628, respectively; SEQ ID NO: 631, 632, 633, 634, 635, and 636, respectively; SEQ ID NO: 639, 640, 641, 642, 643, and 644, respectively; SEQ ID NO: 647, 648, 649, 650, 651, and 652, respectively; SEQ ID NO: 655, 656, 657, 658, 659, and 660, respectively; SEQ ID NO: 663, 664, 665, 666, 667, and 668, respectively; SEQ ID NO: 671, 672, 673, 674, 675, and 676, respectively; SEQ ID NO: 679, 680, 681, 682, 683, and 684, respectively; SEQ ID NO: 687, 688, 689, 690, 691, and 692, respectively; SEQ ID NO: 695, 696, 697, 698, 699, and 700, respectively; SEQ ID NO: 705, 706, 707, 708, 709, and 710, respectively; SEQ ID NO: 725, 726, 727, 728, 729, and 730, respectively; SEQ ID NO: 733, 734, 735, 736, 737, and 738, respectively; SEQ ID NO: 741, 742, 743, 744, 745, and 746, respectively; SEQ ID NO: 749, 750, 751, 752, 753, and 754, respectively; SEQ ID NO: 759, 760, 761, 762, 763, and 764, respectively; SEQ ID NO: 767, 768, 769, 770, 771, and 772, respectively; SEQ ID NO: 775, 776, 777, 778, 779, and 780, respectively; SEQ ID NO: 783, 784, 785, 786, 787, and 788, respectively; SEQ ID NO: 791, 792, 793, 794, 795, and 796, respectively; SEQ ID NO: 799, 800, 801, 802, 803, and 804, respectively; SEQ ID NO: 807, 808, 809, 810, 811, and 812, respectively; SEQ ID NO: 815, 816, 817, 818, 819, and 820, respectively; SEQ ID NO: 823, 824, 825, 826, 827, and 828, respectively; SEQ ID NO: 831, 832, 833, 834, 835, and 836, respectively; SEQ ID NO: 839, 840, 841, 842, 843, and 844, respectively; SEQ ID NO: 847, 848, 849, 850, 851, and 852, respectively; SEQ ID NO: 855, 856, 857, 858, 859, and 860, respectively; SEQ ID NO: 863, 864, 865, 866, 867, and 868, respectively; SEQ ID NO: 871, 872, 873, 874, 875, and 876, respectively; SEQ ID NO: 879, 880, 881, 882, 883, and 884, respectively; SEQ ID NO: 887, 888, 889, 890, 891, and 892, respectively; SEQ ID NO: 895, 896, 897, 898, 899, and 900, respectively; SEQ ID NO: 903, 904, 905, 906, 907, and 908, respectively; SEQ ID NO: 911, 912, 913, 914, 915, and 916, respectively; SEQ ID NO: 919, 920, 921, 922, 923, and 924, respectively; SEQ ID NO: 927, 928, 929, 930, 931, and 932, respectively; SEQ ID NO: 935, 936, 937, 938, 939, and 940, respectively; SEQ ID NO: 945, 946, 947, 948, 949, and 950, respectively; SEQ ID NO: 953, 954, 955, 956, 957, and 958, respectively; SEQ ID NO: 961, 962, 963, 964, 965, and 966, respectively; SEQ ID NO: 969, 970, 971, 972, 973, and 974, respectively; SEQ ID NO: 977, 978, 979, 980, 981, and 982, respectively; SEQ ID NO: 985, 986, 987, 988, 989, and 990, respectively; SEQ ID NO: 997, 998, 999, 1000, 1001, and 1002, respectively; SEQ ID NO: 1005, 1006, 1007, 1008, 1009, and 1010, respectively; SEQ ID NO: 1013, 1014, 1015, 1016, 1017, and 1018, respectively; SEQ ID NO: 1021, 1022, 1023, 1024, 1025, and 1026, respectively; SEQ ID NO: 1029, 1030, 1031, 1032, 1033, and 1034, respectively; or SEQ ID NO: 1037, 1038, 1039, 1040, 1041, and 1042, respectively.

In some cases, an antibody of the present disclosure targets an immune checkpoint selected from the group consisting of PDL1, B7H4, B7H3, and TIGIT. For example, in some cases, an antibody of the present disclosure comprises CDR-H1, CDR-H2, CDR-H3, CDR-L1, CDR-L2, and CDR-L3 comprising at least 80% sequence identity to: SEQ ID NO: 13, 14, 15, 16, 17, and 18, respectively; SEQ ID NO: 77, 78, 79, 80, 81, and 82, respectively; SEQ ID NO: 91, 92, 93, 94, 95, and 96, respectively; SEQ ID NO: 99, 100, 101, 102, 103, and 104, respectively; SEQ ID NO: 107, 108, 109, 110, 111, and 112, respectively; SEQ ID NO: 115, 116, 117, 118, 119, and 120, respectively; SEQ ID NO: 123, 124, 125, 126, 127, and 128, respectively; SEQ ID NO: 131, 132, 133, 134, 135, and 136, respectively; SEQ ID NO: 139, 140, 141, 142, 143, and 144, respectively; SEQ ID NO: 147, 148, 149, 150, 151, and 152, respectively; SEQ ID NO: 155, 156, 157, 158, 159, and 160, respectively; SEQ ID NO: 163, 164, 165, 166, 167, and 168, respectively; SEQ ID NO: 171, 172, 173, 174, 175, and 176, respectively; SEQ ID NO: 179, 180, 181, 182, 183, and 184, respectively; SEQ ID NO: 187, 188, 189, 190, 191, and 192, respectively; SEQ ID NO: 195, 196, 197, 198, 199, and 200, respectively; SEQ ID NO: 203, 204, 205, 206, 207 and 208, respectively; SEQ ID NO: 211, 212, 213, 214, 215, and 216, respectively; SEQ ID NO: 219, 220, 221, 222, 223, and 224, respectively; SEQ ID NO: 227, 228, 229, 230, 231, and 232, respectively; SEQ ID NO: 235, 236, 237, 238, 239, and 240, respectively; SEQ ID NO: 243, 244, 245, 246, 247, and 248, respectively; SEQ ID NO: 623, 624, 625, 626, 627, and 628, respectively; SEQ ID NO: 631, 632, 633, 634, 635, and 636, respectively; SEQ ID NO: 639, 640, 641, 642, 643, and 644, respectively; SEQ ID NO: 647, 648, 649, 650, 651, and 652, respectively; SEQ ID NO: 655, 656, 657, 658, 659, and 660, respectively; SEQ ID NO: 663, 664, 665, 666, 667, and 668, respectively; SEQ ID NO: 671, 672, 673, 674, 675, and 676, respectively; SEQ ID NO: 679, 680, 681, 682, 683, and 684, respectively; SEQ ID NO: 687, 688, 689, 690, 691, and 692, respectively; SEQ ID NO: 695, 696, 697, 698, 699, and 700, respectively; SEQ ID NO: 705, 706, 707, 708, 709, and 710, respectively; or SEQ ID NO: 1021, 1022, 1023, 1024, 1025, and 1026, respectively.

In some cases, the CDR-H1, CDR-H2, CDR-H3, CDR-L1, CDR-L2, and CDR-L3 comprise at least 85% sequence identity to the respective 6 indicated sequences. In some cases, the CDR-H1, CDR-H2, CDR-H3, CDR-L1, CDR-L2, and CDR-L3 each comprise at least 90% sequence identity to the respective 6 indicated sequences. In some cases, the CDR-H1, CDR-H2, CDR-H3, CDR-L1, CDR-L2, and CDR-L3 each comprise at least 95% sequence identity to the respective 6 indicated sequences. In some cases, the CDR-H1, CDR-H2, CDR-H3, CDR-L1, CDR-L2, and CDR-L3 each comprise at most one mutation relative to the respective 6 indicated sequences.

(iv) Exemplary Antibodies

In some cases, an antibody provided herein binds to PDL1. PDL1 is expressed in a broad range of cancers, as well as certain immune cells, and often serves as a primary mechanism for immune suppression in tumor microenvironments. The PDL1 agonism of PD-1 can act as an immune checkpoint, diminishing lymphocyte tumor infiltration, and T-cell receptor mediated proliferation and signaling. While PDL1 antagonism can reverse immune suppression, PDL1 targeting can also localize treatments to the sites of cancers, allowing drugs to selectively target cancer cells or stimulate immune responses in the presence of tumors.

In some cases, an anti-PDL1 antibody comprises CDR-H1, CDR-H2, CDR-H3, CDR-L1, CDR-L2, and CDR-L3 comprising at least 80% sequence identity, at least 85% sequence identity, at least 90% sequence identity, at least 95%, at least 98%, or at least 99% sequence identity to the amino acid sequences of SEQ ID NOs: 13, 14, 15, 16, 17, and 18 respectively. In some cases, the antibody comprises CDR-H1, CDR-H2, CDR-H3, CDR-L1, CDR-L2, and CDR-L3 each comprising at most one mutation relative to the amino acid sequences of SEQ ID NOs: 13, 14, 15, 16, 17, and 18, respectively.

In some embodiments, the antibody comprises a heavy chain comprising an amino acid sequence that is at least 80%, at least 85%, at least 90%, at least 95%, at least 98%, or 99% identical to the amino acid sequence of any one of SEQ ID NO: 1-4 and a light chain comprising an amino acid sequence that is at least 95% at least 96%, at least 97%, at least 98%, at least 99%, or 100% identical to the amino acid sequence of SEQ ID NO: 5. In some embodiments, the antibody comprises a heavy chain comprising an amino acid sequence that is at least 80%, at least 85%, at least 90%, at least 95%, at least 98%, or 99% identical to the amino acid sequence of SEQ ID NO: 1 and a light chain comprising an amino acid sequence that is at least 95% at least 96%, at least 97%, at least 98%, at least 99%, or 100% identical to the amino acid sequence of SEQ ID NO: 5. In some embodiments, the antibody comprises a heavy chain comprising an amino acid sequence that is at least 80%, at least 85%, at least 90%, at least 95%, at least 98%, or 99% identical to the amino acid sequence of SEQ ID NO: 2 and a light chain comprising an amino acid sequence that is at least 95% at least 96%, at least 97%, at least 98%, at least 99%, or 100% identical to the amino acid sequence of SEQ ID NO: 5. In some embodiments, the antibody comprises a heavy chain comprising an amino acid sequence that is at least 80%, at least 85%, at least 90%, at least 95%, at least 98%, or 99% identical to the amino acid sequence of SEQ ID NO: 3 and a light chain comprising an amino acid sequence that is at least 95% at least 96%, at least 97%, at least 98%, at least 99%, or 100% identical to the amino acid sequence of SEQ ID NO: 5. In some embodiments, the antibody comprises a heavy chain comprising an amino acid sequence that is at least 80%, at least 85%, at least 90%, at least 95%, at least 98%, or 99% identical to the amino acid sequence of SEQ ID NO: 4 and a light chain comprising an amino acid sequence that is at least 95% at least 96%, at least 97%, at least 98%, at least 99%, or 100% identical to the amino acid sequence of SEQ ID NO: 5.

In some embodiments, the antibody comprises a heavy chain variable region comprising an amino acid sequence that is at least 80%, at least 85%, at least 90%, at least 95%, at least 98%, or 99% identical to the amino acid sequence of any one of SEQ ID NO: 6-9 and a light chain variable region comprising an amino acid sequence that is at least 95% at least 96%, at least 97%, at least 98%, at least 99%, or 100% identical to the amino acid sequence of SEQ ID NO: 10. In some embodiments, the antibody comprises a heavy chain variable region comprising an amino acid sequence that is at least 80%, at least 85%, at least 90%, at least 95%, at least 98%, or 99% identical to the amino acid sequence of any one of SEQ ID NO: 6 and a light chain variable region comprising an amino acid sequence that is at least 95% at least 96%, at least 97%, at least 98%, at least 99%, or 100% identical to the amino acid sequence of SEQ ID NO: 10. In some embodiments, the antibody comprises a heavy chain variable region comprising an amino acid sequence that is at least 80%, at least 85%, at least 90%, at least 95%, at least 98%, or 99% identical to the amino acid sequence of any one of SEQ ID NO: 7 and a light chain variable region comprising an amino acid sequence that is at least 95% at least 96%, at least 97%, at least 98%, at least 99%, or 100% identical to the amino acid sequence of SEQ ID NO: 10. In some embodiments, the antibody comprises a heavy chain variable region comprising an amino acid sequence that is at least 80%, at least 85%, at least 90%, at least 95%, at least 98%, or 99% identical to the amino acid sequence of any one of SEQ ID NO: 8 and a light chain variable region comprising an amino acid sequence that is at least 95% at least 96%, at least 97%, at least 98%, at least 99%, or 100% identical to the amino acid sequence of SEQ ID NO: 10. In some embodiments, the antibody comprises a heavy chain variable region comprising an amino acid sequence that is at least 80%, at least 85%, at least 90%, at least 95%, at least 98%, or 99% identical to the amino acid sequence of any one of SEQ ID NO: 9 and a light chain variable region comprising an amino acid sequence that is at least 95% at least 96%, at least 97%, at least 98%, at least 99%, or 100% identical to the amino acid sequence of SEQ ID NO: 10.

In some embodiments, the antibody comprises a heavy chain variable region comprising an amino acid sequence that is at least 80%, at least 85%, at least 90%, at least 95%, at least 98%, or 99% identical to the amino acid sequence of SEQ ID NO: 19 and a light chain variable region comprising an amino acid sequence that is at least 95% at least 96%, at least 97%, at least 98%, at least 99%, or 100% identical to the amino acid sequence of SEQ ID NO: 20.

In some embodiments, an antibody provided herein binds to EphA2. In some embodiments, the antibody comprises CDR-H1, CDR-H2, CDR-H3, CDR-L1, CDR-L2, and CDR-L3 comprising the amino acid sequences that are at least 80%, at least 85%, at least 90%, at least 95%, at least 98%, or at least 99% identical to the amino acid sequences of SEQ ID NOs: 21, 22, 23, 24, 25, and 26, respectively.

In some embodiments, the anti-EphA2 antibody comprises a heavy chain variable region comprising an amino acid sequence that is at least 80%, at least 85%, at least 90%, at least 95%, at least 98%, or at least 99% identical to the amino acid sequence of SEQ ID NO: 27 and a light chain variable region comprising an amino acid sequence that is at least 80%, at least 85%, at least 90%, at least 95%, at least 98%, or at least 99% identical to the amino acid sequence of SEQ ID NO: 28. In some embodiments, the anti-EphA2 antibody comprises a heavy chain comprising an amino acid sequence that is at least 80%, at least 85%, at least 90%, at least 95%, at least 98%, or at least 99% identical to the amino acid sequence of SEQ ID NO: 29 or SEQ ID NO: 30 and a light chain comprising an amino acid sequence that is at least 80%, at least 85%, at least 90%, at least 95%, at least 98%, or at least 99% identical to the amino acid sequence of the amino acid sequence of SEQ ID NO: 31. In some embodiments, the anti-EphA2 antibody comprises a heavy chain comprising the amino acid sequence that is at least 80%, at least 85%, at least 90%, at least 95%, at least 98%, or at least 99% identical to the amino acid sequence of SEQ ID NO: 32 or SEQ ID NO: 33 and a light chain comprising the amino acid sequence of SEQ ID NO: 34. In some embodiments, the anti-EphA2 antibody comprises a heavy chain that is at least 80%, at least 85%, at least 90%, at least 95%, at least 98%, or at least 99% identical to the amino acid sequence of SEQ ID NO: 35 or SEQ ID NO: 36 and a light chain comprising the amino acid sequence of SEQ ID NO: 37. In some embodiments, the antibody is h1C1 or 1C1.

In some embodiments, the anti-EphA2 antibody comprises a heavy chain variable region comprising an amino acid sequence that is at least 80%, at least 85%, at least 90%, at least 95%, at least 98%, or 99% identical to the amino acid sequence of SEQ ID NO: 27 and a light chain variable region comprising an amino acid sequence that is at least 80% at least 85%, at least 90%, at least 95%, at least 98%, or 99% identical to the amino acid sequence of SEQ ID NO: 28.

In some cases, an antibody provided herein binds to CD30. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 38, 39, 40, 41, 42, and 43, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 38, 39, 40, 41, 42, and 43, respectively. In some embodiments, the anti-CD30 antibody comprises a heavy chain comprising an amino acid sequence that is at least 80%, at least 85%, at least 90%, at least 95%, at least 98%, or 99% identical to the amino acid sequence of either SEQ ID NO: 46 or 47 and a light chain comprising an amino acid sequence that is at least 80% at least 85%, at least 90%, at least 95%, at least 98%, or 99% identical to the amino acid sequence of SEQ ID NO: 48. In some embodiments, the anti-CD30 antibody comprises a heavy chain variable region comprising an amino acid sequence that is at least 80%, at least 85%, at least 90%, at least 95%, at least 98%, or 99% identical to the amino acid sequence of SEQ ID NO: 44 and a light chain variable region comprising an amino acid sequence that is at least 80% at least 85%, at least 90%, at least 95%, at least 98%, or 99% identical to the amino acid sequence of SEQ ID NO: 45.

In some cases, an antibody provided herein binds to CD228. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 49, 50, 51, 52, 53, and 54, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 49, 50, 51, 52, 53, and 54, respectively. In some embodiments, the anti-CD228 antibody comprises a heavy chain variable region comprising an amino acid sequence that is at least 80%, at least 85%, at least 90%, at least 95%, at least 98%, or 99% identical to the amino acid sequence of SEQ ID NO: 55 and a light chain variable region comprising an amino acid sequence that is at least 80% at least 85%, at least 90%, at least 95%, at least 98%, or 99% identical to the amino acid sequence of SEQ ID NO: 56. In some embodiments, the anti-CD228 antibody comprises a heavy chain comprising an amino acid sequence that is at least 80%, at least 85%, at least 90%, at least 95%, at least 98%, or 99% identical to the amino acid sequence of either of SEQ ID NO: 57 or 58 and a light chain comprising an amino acid sequence that is at least 80% at least 85%, at least 90%, at least 95%, at least 98%, or 99% identical to the amino acid sequence of SEQ ID NO: 59. In some embodiments, the anti-CD228 antibody comprises a heavy chain comprising an amino acid sequence that is at least 80%, at least 85%, at least 90%, at least 95%, at least 98%, or 99% identical to the amino acid sequence of either of SEQ ID NO: 60 or 61 and a light chain comprising an amino acid sequence that is at least 80% at least 85%, at least 90%, at least 95%, at least 98%, or 99% identical to the amino acid sequence of SEQ ID NO: 62.

In some cases, an antibody provided herein binds to avB6. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 63, 64, 65, 66, 67, and 68, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 63, 64, 65, 66, 67, and 68, respectively. In some embodiments, the anti-H2A2 antibody comprises a heavy chain variable region comprising an amino acid sequence that is at least 80%, at least 85%, at least 90%, at least 95%, at least 98%, or 99% identical to the amino acid sequence of SEQ ID NO: 69 and a light chain variable region comprising an amino acid sequence that is at least 80% at least 85%, at least 90%, at least 95%, at least 98%, or 99% identical to the amino acid sequence of SEQ ID NO: 70. In some embodiments, the anti-H2A2 antibody comprises a heavy chain comprising an amino acid sequence that is at least 80%, at least 85%, at least 90%, at least 95%, at least 98%, or 99% identical to the amino acid sequence of either SEQ ID NO: 71 or 72 and a light chain comprising an amino acid sequence that is at least 80% at least 85%, at least 90%, at least 95%, at least 98%, or 99% identical to the amino acid sequence of SEQ ID NO: 73. In some embodiments, the anti-H2A2 antibody comprises a heavy chain comprising an amino acid sequence that is at least 80%, at least 85%, at least 90%, at least 95%, at least 98%, or 99% identical to the amino acid sequence of either SEQ ID NO: 74 or 75 and a light chain comprising an amino acid sequence that is at least 80% at least 85%, at least 90%, at least 95%, at least 98%, or 99% identical to the amino acid sequence of SEQ ID NO: 76. In some embodiments, the anti-avB6 antibody comprises a heavy chain variable region comprising an amino acid sequence that is at least 80%, at least 85%, at least 90%, at least 95%, at least 98%, or 99% identical to the amino acid sequence of SEQ ID NO: 69 and a light chain variable region comprising an amino acid sequence that is at least 80% at least 85%, at least 90%, at least 95%, at least 98%, or 99% identical to the amino acid sequence of SEQ ID NO: 70. In some embodiments, the anti-avB6 antibody comprises a heavy chain comprising an amino acid sequence that is at least 80%, at least 85%, at least 90%, at least 95%, at least 98%, or 99% identical to the amino acid sequence of either SEQ ID NO: 71 or 72 and a light chain comprising an amino acid sequence that is at least 80% at least 85%, at least 90%, at least 95%, at least 98%, or 99% identical to the amino acid sequence of SEQ ID NO: 73. In some embodiments, the anti-avB6 antibody comprises a heavy chain comprising an amino acid sequence that is at least 80%, at least 85%, at least 90%, at least 95%, at least 98%, or 99% identical to the amino acid sequence of either SEQ ID NO: 74 or 75 and a light chain comprising an amino acid sequence that is at least 80% at least 85%, at least 90%, at least 95%, at least 98%, or 99% identical to the amino acid sequence of SEQ ID NO: 76.

In some cases, an antibody provided herein binds to B7H4. In some cases, the antibody comprises a set of CDR sequences (CDR-H1, CDR-H2, CDR-H3, CDR-L1, CDR-L2, and CDR-L3, respectively) of which each sequence comprises at least 80% sequence identity, at least 85% sequence identity, at least 90% sequence identity, at least 95%, or 100% sequence identity to amino acid sequences from a set of amino acid sequences selected from the group consisting of SEQ ID NOs: 77-82, SEQ ID NOs: 91-96, SEQ ID NOs: 99-104, SEQ ID NOs: 107-112, SEQ ID NOs: 115-120, SEQ ID NOs: 123-128, SEQ ID NOs: 131-136, SEQ ID NOs: 139-144, SEQ ID NOs: 147-152, SEQ ID NOs: 155-160, SEQ ID NOs: 163-168, SEQ ID NOs: 171-176, SEQ ID NOs: 179-184, SEQ ID NOs: 187-192, SEQ ID NOs: 195-200, SEQ ID NOs: 203-208, SEQ ID NOs: 211-216, SEQ ID NOs: 219-224, SEQ ID NOs: 227-232, SEQ ID NOs: 235-240, and SEQ ID NOs: 243-248. In some cases, the antibody comprises a set of CDR sequences (CDR-H1, CDR-H2, CDR-H3, CDR-L1, CDR-L2, and CDR-L3, respectively) each comprising at most one mutation relative to an amino acid sequence from a set of amino acid sequences selected from the group consisting of SEQ ID NOs: 77-82, SEQ ID NOs: 91-96, SEQ ID NOs: 99-104, SEQ ID NOs: 107-112, SEQ ID NOs: 115-120, SEQ ID NOs: 123-128, SEQ ID NOs: 131-136, SEQ ID NOs: 139-144, SEQ ID NOs: 147-152, SEQ ID NOs: 155-160, SEQ ID NOs: 163-168, SEQ ID NOs: 171-176, SEQ ID NOs: 179-184, SEQ ID NOs: 187-192, SEQ ID NOs: 195-200, SEQ ID NOs: 203-208, SEQ ID NOs: 211-216, SEQ ID NOs: 219-224, SEQ ID NOs: 227-232, SEQ ID NOs: 235-240, and SEQ ID NOs: 243-248. In some cases, the anti-B7H4 antibody comprises a heavy chain and a light chain comprising amino acid sequences that are at least 80%, at least 85%, at least 90%, at least 95%, at least 98%, at least 99%, or at least 100% identical to the amino acid sequences of SEQ ID NO: 85 and 87, SEQ ID NO: 86 and 87, SEQ ID NO: 88 and 90, SEQ ID NO: 89 and 90, SEQ ID NO: 251 and 252, SEQ ID NO: 253 and 254, SEQ ID NO: 255 and 256, SEQ ID NO: 257 and 258, SEQ ID NO: 259 and 260, SEQ ID NO: 261 and 262, SEQ ID NO: 263 and 264, SEQ ID NO: 265 and 266, SEQ ID NO: 267 and 268, SEQ ID NO: 269 and 270, SEQ ID NO: 271 and 272, SEQ ID NO: 273 and 274, SEQ ID NO: 275 and 276, SEQ ID NO: 277 and 278, SEQ ID NO: 279 and 280, SEQ ID NO: 281 and 282, SEQ ID NO: 283 and 284, SEQ ID NO: 285 and 286, SEQ ID NO: 287 and 288, or SEQ ID NO: 289 and 290, respectively. In some embodiments, the anti-B7H4 antibody comprises a heavy chain variable region and a light chain variable region comprising amino acid sequences that are at least 80%, at least 85%, at least 90%, at least 95%, at least 98%, at least 99%, or at least 100% identical to the amino acid sequences of SEQ ID NO: 83 and 84, SEQ ID NO: 97 and 98, SEQ ID NO: 105 and 106, SEQ ID NO: 113 and 114, SEQ ID NO: 121 and 122, SEQ ID NO: 129 and 130, SEQ ID NO: 137 and 138, SEQ ID NO: 153 and 154, SEQ ID NO: 161 and 162, SEQ ID NO: 169 and 170, SEQ ID NO: 177 and 178, SEQ ID NO: 185 and 186, SEQ ID NO: 193 and 194, SEQ ID NO: 201 and 202, SEQ ID NO: 209 and 210, SEQ ID NO: 217 and 218, SEQ ID NO: 225 and 226, SEQ ID NO: 233 and 234, SEQ ID NO: 241 and 242, or SEQ ID NO: 249 and 250, respectively.

In some cases, an antibody provided herein binds to CD70. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 291, 292, 293, 294, 295, and 296, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 291, 292, 293, 294, 295, and 296, respectively. In some embodiments, the anti-CD70 antibody comprises a heavy chain variable region comprising an amino acid sequence that is at least 80%, at least 85%, at least 90%, at least 95%, at least 98%, or 99% identical to the amino acid sequence of SEQ ID NO: 297 and a light chain variable region comprising an amino acid sequence that is at least 80% at least 85%, at least 90%, at least 95%, at least 98%, or 99% identical to the amino acid sequence of SEQ ID NO: 298. In some embodiments, the anti-CD70 antibody comprises a heavy chain comprising an amino acid sequence that is at least 80%, at least 85%, at least 90%, at least 95%, at least 98%, or 99% identical to the amino acid sequence of SEQ ID NO: 299 and a light chain comprising an amino acid sequence that is at least 80% at least 85%, at least 90%, at least 95%, at least 98%, or 99% identical to the amino acid sequence of SEQ ID NO: 300.

In some cases, an antibody provided herein binds to TROP2. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 301, 302, 303, 304, 305, and 306, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 301, 302, 303, 304, 305, and 306, respectively. In some cases, an antibody provided herein binds to TROP2. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 309, 310, 311, 312, 313, and 314 respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 309, 310, 311, 312, 313, and 314, respectively. In some embodiments, the anti-TROP2 antibody comprises a heavy chain variable region comprising an amino acid sequence that is at least 80%, at least 85%, at least 90%, at least 95%, at least 98%, or 99% identical to the amino acid sequence of SEQ ID NO: 307 and a light chain variable region comprising an amino acid sequence that is at least 80% at least 85%, at least 90%, at least 95%, at least 98%, or 99% identical to the amino acid sequence of SEQ ID NO: 308. In some embodiments, the anti-TROP2 antibody comprises a heavy chain variable region comprising an amino acid sequence that is at least 80%, at least 85%, at least 90%, at least 95%, at least 98%, or 99% identical to the amino acid sequence of SEQ ID NO: 315 and a light chain variable region comprising an amino acid sequence that is at least 80% at least 85%, at least 90%, at least 95%, at least 98%, or 99% identical to the amino acid sequence of SEQ ID NO: 316.

In some cases, an antibody provided herein binds to MICA. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 317, 318, 319, 320, 321, and 322, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 317, 318, 319, 320, 321, and 322, respectively. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 325, 326, 327, 328, 329, and 330, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 325, 326, 327, 328, 329, and 330, respectively. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 333, 334, 335, 336, 337, and 338, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 333, 334, 335, 336, 337, and 338, respectively. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 341, 342, 343, 344, 345, and 346, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 341, 342, 343, 344, 345, and 346, respectively. In some embodiments, the anti-MICA antibody comprises a heavy chain variable region comprising an amino acid sequence that is at least 80%, at least 85%, at least 90%, at least 95%, at least 98%, or 99% identical to the amino acid sequence of SEQ ID NO: 323 and a light chain variable region comprising an amino acid sequence that is at least 80% at least 85%, at least 90%, at least 95%, at least 98%, or 99% identical to the amino acid sequence of SEQ ID NO: 324. In some embodiments, the anti-MICA antibody comprises a heavy chain variable region comprising an amino acid sequence that is at least 80%, at least 85%, at least 90%, at least 95%, at least 98%, or 99% identical to the amino acid sequence of SEQ ID NO: 331 and a light chain variable region comprising an amino acid sequence that is at least 80% at least 85%, at least 90%, at least 95%, at least 98%, or 99% identical to the amino acid sequence of SEQ ID NO: 332. In some embodiments, the anti-MICA antibody comprises a heavy chain variable region comprising an amino acid sequence that is at least 80%, at least 85%, at least 90%, at least 95%, at least 98%, or 99% identical to the amino acid sequence of SEQ ID NO: 340 and a light chain variable region comprising an amino acid sequence that is at least 80% at least 85%, at least 90%, at least 95%, at least 98%, or 99% identical to the amino acid sequence of SEQ ID NO: 340. In some embodiments, the anti-MICA antibody comprises a heavy chain variable region comprising an amino acid sequence that is at least 80%, at least 85%, at least 90%, at least 95%, at least 98%, or 99% identical to the amino acid sequence of SEQ ID NO: 347 and a light chain variable region comprising an amino acid sequence that is at least 80% at least 85%, at least 90%, at least 95%, at least 98%, or 99% identical to the amino acid sequence of SEQ ID NO: 348.

In some cases, an antibody provided herein binds to CD51. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 349, 350, 351, 352, 353, and 354, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 349, 350, 351, 352, 353, and 354, respectively. In some cases, an antibody provided herein binds to CD51. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 357, 358, 359, 360, 361, and 362, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 357, 358, 359, 360, 361, and 362, respectively. In some embodiments, the anti-CD51 antibody comprises a heavy chain variable region comprising an amino acid sequence that is at least 80%, at least 85%, at least 90%, at least 95%, at least 98%, or 99% identical to the amino acid sequence of SEQ ID NO: 355 and a light chain variable region comprising an amino acid sequence that is at least 80% at least 85%, at least 90%, at least 95%, at least 98%, or 99% identical to the amino acid sequence of SEQ ID NO: 356. In some embodiments, the anti-CD51 antibody comprises a heavy chain variable region comprising an amino acid sequence that is at least 80%, at least 85%, at least 90%, at least 95%, at least 98%, or 99% identical to the amino acid sequence of SEQ ID NO: 363 and a light chain variable region comprising an amino acid sequence that is at least 80% at least 85%, at least 90%, at least 95%, at least 98%, or 99% identical to the amino acid sequence of SEQ ID NO: 364.

In some cases, an antibody provided herein binds to gpA33. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 365, 366, 367, 368, 369, and 370, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 365, 366, 367, 368, 369, and 370, respectively. In some embodiments, the anti-gpA33 antibody comprises a heavy chain variable region comprising an amino acid sequence that is at least 80%, at least 85%, at least 90%, at least 95%, at least 98%, or 99% identical to the amino acid sequence of SEQ ID NO: 371 and a light chain variable region comprising an amino acid sequence that is at least 80% at least 85%, at least 90%, at least 95%, at least 98%, or 99% identical to the amino acid sequence of SEQ ID NO: 372.

In some cases, an antibody provided herein binds to IL1Rap. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 373, 374, 375, 376, 377, and 378, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 373, 374, 375, 376, 377, and 378, respectively. In some embodiments, the anti-IL1Rap antibody comprises a heavy chain variable region comprising an amino acid sequence that is at least 80%, at least 85%, at least 90%, at least 95%, at least 98%, or 99% identical to the amino acid sequence of SEQ ID NO: 379 and a light chain variable region comprising an amino acid sequence that is at least 80% at least 85%, at least 90%, at least 95%, at least 98%, or 99% identical to the amino acid sequence of SEQ ID NO: 380.

In some cases, an antibody provided herein binds to EpCAM. In some cases, the antibody comprises a set of CDR sequences (CDR-H1, CDR-H2, CDR-H3, CDR-L1, CDR-L2, and CDR-L3, respectively) of which each sequence comprises at least 80% sequence identity, at least 85% sequence identity, at least 90% sequence identity, at least 95%, or 100% sequence identity to amino acid sequences from a set of amino acid sequences selected from the group consisting of SEQ ID NOs: 381-386, SEQ ID NOs: 389-394, SEQ ID NOs: 397-402, SEQ ID NOs: 405-410, SEQ ID NOs: 413-418, or SEQ ID NOs: 421-426. In some embodiments, the anti-EpCAM antibody comprises a heavy chain variable region and a light chain variable region comprising amino acid sequences that are at least 80%, at least 85%, at least 90%, at least 95%, at least 98%, at least 99%, or at least 100% identical to the amino acid sequences of SEQ ID NO: 387 and 388, SEQ ID NO: 395 and 396, SEQ ID NO: 403 and 404, SEQ ID NO: 411 and 412, SEQ ID NO: 419 and 420, or SEQ ID NO: 427 and 428, respectively.

In some cases, an antibody provided herein binds to CD352. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 429, 430, 431, 432, 433, and 434, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 429, 430, 431, 432, 433, and 434, respectively. In some embodiments, the anti-CD352 antibody comprises a heavy chain variable region comprising an amino acid sequence that is at least 80%, at least 85%, at least 90%, at least 95%, at least 98%, or 99% identical to the amino acid sequence of SEQ ID NO: 435 and a light chain variable region comprising an amino acid sequence that is at least 80% at least 85%, at least 90%, at least 95%, at least 98%, or 99% identical to the amino acid sequence of SEQ ID NO: 436.

In some cases, an antibody provided herein binds to CS1. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 437, 438, 439, 440, 441, and 442, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 437, 438, 439, 440, 441, and 442, respectively. In some embodiments, the anti-CS1 antibody comprises a heavy chain variable region comprising an amino acid sequence that is at least 80%, at least 85%, at least 90%, at least 95%, at least 98%, or 99% identical to the amino acid sequence of SEQ ID NO: 443 and a light chain variable region comprising an amino acid sequence that is at least 80% at least 85%, at least 90%, at least 95%, at least 98%, or 99% identical to the amino acid sequence of SEQ ID NO: 444.

In some cases, an antibody provided herein binds to CD38. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 445, 446, 447, 448, 449, and 450, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 445, 446, 447, 448, 449, and 450, respectively. In some embodiments, the anti-CD38 antibody comprises a heavy chain variable region comprising an amino acid sequence that is at least 80%, at least 85%, at least 90%, at least 95%, at least 98%, or 99% identical to the amino acid sequence of SEQ ID NO: 451 and a light chain variable region comprising an amino acid sequence that is at least 80% at least 85%, at least 90%, at least 95%, at least 98%, or 99% identical to the amino acid sequence of SEQ ID NO: 452.

In some cases, an antibody provided herein binds to CD25. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 453, 454, 455, 456, 457, and 458, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 453, 454, 455, 456, 457, and 458, respectively. In some embodiments, the anti-CD25 antibody comprises a heavy chain variable region comprising an amino acid sequence that is at least 80%, at least 85%, at least 90%, at least 95%, at least 98%, or 99% identical to the amino acid sequence of SEQ ID NO: 459 and a light chain variable region comprising an amino acid sequence that is at least 80% at least 85%, at least 90%, at least 95%, at least 98%, or 99% identical to the amino acid sequence of SEQ ID NO: 460.

In some cases, an antibody provided herein binds to ADAM9. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 461, 462, 463, 464, 465, and 466, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 461, 462, 463, 464, 465, and 466, respectively. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 469, 470, 471, 472, 473, and 474, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 469, 470, 471, 472, 473, and 474, respectively. In some embodiments, the anti-ADAM9 antibody comprises a heavy chain variable region comprising an amino acid sequence that is at least 80%, at least 85%, at least 90%, at least 95%, at least 98%, or 99% identical to the amino acid sequence of SEQ ID NO: 467 and a light chain variable region comprising an amino acid sequence that is at least 80% at least 85%, at least 90%, at least 95%, at least 98%, or 99% identical to the amino acid sequence of SEQ ID NO: 468. In some embodiments, the anti-ADAM9 antibody comprises a heavy chain variable region comprising an amino acid sequence that is at least 80%, at least 85%, at least 90%, at least 95%, at least 98%, or 99% identical to the amino acid sequence of SEQ ID NO: 475 and a light chain variable region comprising an amino acid sequence that is at least 80% at least 85%, at least 90%, at least 95%, at least 98%, or 99% identical to the amino acid sequence of SEQ ID NO: 476.

In some cases, an antibody provided herein binds to CD59. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 477, 478, 479, 480, 481, and 482, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 477, 478, 479, 480, 481, and 482, respectively. In some embodiments, the anti-CD59 antibody comprises a heavy chain variable region comprising an amino acid sequence that is at least 80%, at least 85%, at least 90%, at least 95%, at least 98%, or 99% identical to the amino acid sequence of SEQ ID NO: 483 and a light chain variable region comprising an amino acid sequence that is at least 80% at least 85%, at least 90%, at least 95%, at least 98%, or 99% identical to the amino acid sequence of SEQ ID NO: 484.

In some cases, an antibody provided herein binds to CD19. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 485, 486, 487, 488, 489, and 490, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 485, 486, 487, 488, 489, and 490, respectively. In some embodiments, the anti-CD19 antibody comprises a heavy chain variable region comprising an amino acid sequence that is at least 80%, at least 85%, at least 90%, at least 95%, at least 98%, or 99% identical to the amino acid sequence of SEQ ID NO: 491 and a light chain variable region comprising an amino acid sequence that is at least 80% at least 85%, at least 90%, at least 95%, at least 98%, or 99% identical to the amino acid sequence of SEQ ID NO: 492. In some embodiments, the anti-CD19 antibody comprises a heavy chain comprising an amino acid sequence that is at least 80%, at least 85%, at least 90%, at least 95%, at least 98%, or 99% identical to the amino acid sequence of SEQ ID NO: 493 and a light chain comprising an amino acid sequence that is at least 80% at least 85%, at least 90%, at least 95%, at least 98%, or 99% identical to the amino acid sequence of SEQ ID NO: 494.

In some cases, an antibody provided herein binds to CD138. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 495, 496, 497, 498, 499, and 500, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 495, 496, 497, 498, 499, and 500, respectively. In some embodiments, the anti-CD138 antibody comprises a heavy chain variable region comprising an amino acid sequence that is at least 80%, at least 85%, at least 90%, at least 95%, at least 98%, or 99% identical to the amino acid sequence of SEQ ID NO: 501 and a light chain variable region comprising an amino acid sequence that is at least 80% at least 85%, at least 90%, at least 95%, at least 98%, or 99% identical to the amino acid sequence of SEQ ID NO: 502.

In some cases, an antibody provided herein binds to CD166. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 503, 504, 505, 506, 507, and 508, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 503, 504, 505, 506, 507, and 508, respectively. In some embodiments, the anti-CD166 antibody comprises a heavy chain variable region comprising an amino acid sequence that is at least 80%, at least 85%, at least 90%, at least 95%, at least 98%, or 99% identical to the amino acid sequence of SEQ ID NO: 509 and a light chain variable region comprising an amino acid sequence that is at least 80% at least 85%, at least 90%, at least 95%, at least 98%, or 99% identical to the amino acid sequence of SEQ ID NO: 510.

In some cases, an antibody provided herein binds to CD56. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 511, 512, 513, 514, 515, and 516, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 511, 512, 513, 514, 515, and 516, respectively. In some embodiments, the anti-CD56 antibody comprises a heavy chain variable region comprising an amino acid sequence that is at least 80%, at least 85%, at least 90%, at least 95%, at least 98%, or 99% identical to the amino acid sequence of SEQ ID NO: 517 and a light chain variable region comprising an amino acid sequence that is at least 80% at least 85%, at least 90%, at least 95%, at least 98%, or 99% identical to the amino acid sequence of SEQ ID NO: 518.

In some cases, an antibody provided herein binds to CD74. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 519, 520, 521, 522, 523, and 524, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 519, 520, 521, 522, 523, and 524, respectively. In some embodiments, the anti-CD74 antibody comprises a heavy chain variable region comprising an amino acid sequence that is at least 80%, at least 85%, at least 90%, at least 95%, at least 98%, or 99% identical to the amino acid sequence of SEQ ID NO: 525 and a light chain variable region comprising an amino acid sequence that is at least 80% at least 85%, at least 90%, at least 95%, at least 98%, or 99% identical to the amino acid sequence of SEQ ID NO: 526.

In some cases, an antibody provided herein binds to CEACAM5. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 527, 528, 529, 530, 531, and 532, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 527, 528, 529, 530, 531, and 532, respectively. In some embodiments, the anti-CEACAM5 antibody comprises a heavy chain variable region comprising an amino acid sequence that is at least 80%, at least 85%, at least 90%, at least 95%, at least 98%, or 99% identical to the amino acid sequence of SEQ ID NO: 533 and a light chain variable region comprising an amino acid sequence that is at least 80% at least 85%, at least 90%, at least 95%, at least 98%, or 99% identical to the amino acid sequence of SEQ ID NO: 534.

In some cases, an antibody provided herein binds to CanAg. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 535, 536, 537, 538, 539, and 540, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 535, 536, 537, 538, 539, and 540, respectively. In some embodiments, the anti-CanAg antibody comprises a heavy chain variable region comprising an amino acid sequence that is at least 80%, at least 85%, at least 90%, at least 95%, at least 98%, or 99% identical to the amino acid sequence of SEQ ID NO: 541 and a light chain variable region comprising an amino acid sequence that is at least 80% at least 85%, at least 90%, at least 95%, at least 98%, or 99% identical to the amino acid sequence of SEQ ID NO: 542.

In some cases, an antibody provided herein binds to DLL-3. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 543, 544, 545, 546, 547, and 548, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 543, 544, 545, 546, 547, and 548, respectively. In some embodiments, the anti-DLL-3 antibody comprises a heavy chain variable region comprising an amino acid sequence that is at least 80%, at least 85%, at least 90%, at least 95%, at least 98%, or 99% identical to the amino acid sequence of SEQ ID NO: 549 and a light chain variable region comprising an amino acid sequence that is at least 80% at least 85%, at least 90%, at least 95%, at least 98%, or 99% identical to the amino acid sequence of SEQ ID NO: 550.

In some cases, an antibody provided herein binds to DPEP-3. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 551, 552, 553, 554, 555, and 556, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 551, 552, 553, 554, 555, and 556, respectively. In some embodiments, the anti-DPEP-3 antibody comprises a heavy chain variable region comprising an amino acid sequence that is at least 80%, at least 85%, at least 90%, at least 95%, at least 98%, or 99% identical to the amino acid sequence of SEQ ID NO: 557 and a light chain variable region comprising an amino acid sequence that is at least 80% at least 85%, at least 90%, at least 95%, at least 98%, or 99% identical to the amino acid sequence of SEQ ID NO: 558.

In some cases, an antibody provided herein binds to EGFR. In some cases, the antibody comprises a set of CDR sequences (CDR-H1, CDR-H2, CDR-H3, CDR-L1, CDR-L2, and CDR-L3, respectively) of which each sequence comprises at least 80% sequence identity, at least 85% sequence identity, at least 90% sequence identity, at least 95%, or 100% sequence identity to amino acid sequences from a set of amino acid sequences selected from the group consisting of SEQ ID NOs: 559-564, SEQ ID NOs: 567-572, SEQ ID NOs: 575-580, and SEQ ID NOs: 895-900. In some embodiments, the anti-EGFR antibody comprises a heavy chain variable region and a light chain variable region comprising amino acid sequences that are at least 80%, at least 85%, at least 90%, at least 95%, at least 98%, at least 99%, or at least 100% identical to the amino acid sequences of SEQ ID NO: 565 and 566, SEQ ID NO: 573 and 574, SEQ ID NO: 581 and 582, or SEQ ID NO: 901 and 902, respectively.

In some cases, an antibody provided herein binds to FRa. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 583, 584, 585, 586, 587, and 588, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 583, 584, 585, 586, 587, and 588, respectively. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 591, 592, 593, 594, 595, and 596, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 591, 592, 593, 594, 595, and 596, respectively. In some embodiments, the anti-FRa antibody comprises a heavy chain variable region comprising an amino acid sequence that is at least 80%, at least 85%, at least 90%, at least 95%, at least 98%, or 99% identical to the amino acid sequence of SEQ ID NO: 589 and a light chain variable region comprising an amino acid sequence that is at least 80% at least 85%, at least 90%, at least 95%, at least 98%, or 99% identical to the amino acid sequence of SEQ ID NO: 590. In some embodiments, the anti-FRa antibody comprises a heavy chain variable region comprising an amino acid sequence that is at least 80%, at least 85%, at least 90%, at least 95%, at least 98%, or 99% identical to the amino acid sequence of SEQ ID NO: 597 and a light chain variable region comprising an amino acid sequence that is at least 80% at least 85%, at least 90%, at least 95%, at least 98%, or 99% identical to the amino acid sequence of SEQ ID NO: 598.

In some cases, an antibody provided herein binds to MUC-1. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 599, 600, 601, 602, 603, and 604, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 599, 600, 601, 602, 603, and 604, respectively. In some embodiments, the anti-MUC-1 antibody comprises a heavy chain variable region comprising an amino acid sequence that is at least 80%, at least 85%, at least 90%, at least 95%, at least 98%, or 99% identical to the amino acid sequence of SEQ ID NO: 605 and a light chain variable region comprising an amino acid sequence that is at least 80% at least 85%, at least 90%, at least 95%, at least 98%, or 99% identical to the amino acid sequence of SEQ ID NO: 606.

In some cases, an antibody provided herein binds to Mesothelin. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 607, 608, 609, 610, 611, and 612, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 607, 608, 609, 610, 611, and 612, respectively. In some embodiments, the anti-Mesothelin antibody comprises a heavy chain variable region comprising an amino acid sequence that is at least 80%, at least 85%, at least 90%, at least 95%, at least 98%, or 99% identical to the amino acid sequence of SEQ ID NO: 613 and a light chain variable region comprising an amino acid sequence that is at least 80% at least 85%, at least 90%, at least 95%, at least 98%, or 99% identical to the amino acid sequence of SEQ ID NO: 614.

In some cases, an antibody provided herein binds to ROR-1. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 615, 616, 617, 618, 619, and 620, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 615, 616, 617, 618, 619, and 620, respectively. In some embodiments, the anti-ROR-1 antibody comprises a heavy chain variable region comprising an amino acid sequence that is at least 80%, at least 85%, at least 90%, at least 95%, at least 98%, or 99% identical to the amino acid sequence of SEQ ID NO: 621 and a light chain variable region comprising an amino acid sequence that is at least 80% at least 85%, at least 90%, at least 95%, at least 98%, or 99% identical to the amino acid sequence of SEQ ID NO: 622.

In some cases, an antibody provided herein binds to B7H3. In some cases, the antibody comprises a set of CDR sequences (CDR-H1, CDR-H2, CDR-H3, CDR-L1, CDR-L2, and CDR-L3, respectively) of which each sequence comprises at least 80% sequence identity, at least 85% sequence identity, at least 90% sequence identity, at least 95%, or 100% sequence identity to amino acid sequences from a set of amino acid sequences selected from the group consisting of SEQ ID NOs: 623-628, SEQ ID NOs: 631-636, SEQ ID NOs: 639-644, SEQ ID NOs: 647-652, SEQ ID NOs: 655-660, SEQ ID NOs: 663-668, SEQ ID NOs: 671-676, SEQ ID NOs: 679-684, SEQ ID NOs: 687-692, SEQ ID NOs: 695-700, and SEQ ID NOs: 705-710. In some embodiments, the anti-B7H3 antibody comprises a heavy chain variable region and a light chain variable region comprising amino acid sequences that are at least 80%, at least 85%, at least 90%, at least 95%, at least 98%, at least 99%, or at least 100% identical to the amino acid sequences of SEQ ID NO: 629 and 630, SEQ ID NOs: 637 and 638, SEQ ID NOs: 645 and 646, SEQ ID NOs: 653 and 654, SEQ ID NOs: 661 and 662, SEQ ID NOs: 669 and 670, SEQ ID NOs: 677 and 678, SEQ ID NOs: 685 and 686, SEQ ID NOs: 693 and 694, SEQ ID NOs: 701 and 702, SEQ ID NOs: 703 and 704, SEQ ID NOs: 711 and 712, SEQ ID NOs: 713 and 714, and SEQ ID NOs: 715 and 716, respectively.

In some cases, an antibody provided herein binds to HER3. In some embodiments, the anti-HER3 antibody comprises a heavy chain comprising an amino acid sequence that is at least 80%, at least 85%, at least 90%, at least 95%, at least 98%, or 99% identical to the amino acid sequence of SEQ ID NO: 717 and a light chain comprising an amino acid sequence that is at least 80% at least 85%, at least 90%, at least 95%, at least 98%, or 99% identical to the amino acid sequence of SEQ ID NO: 718. In some cases, an antibody provided herein binds to HER3. In some embodiments, the anti-HER3 antibody comprises a heavy chain comprising an amino acid sequence that is at least 80%, at least 85%, at least 90%, at least 95%, at least 98%, or 99% identical to the amino acid sequence of SEQ ID NO: 719 and a light chain comprising an amino acid sequence that is at least 80% at least 85%, at least 90%, at least 95%, at least 98%, or 99% identical to the amino acid sequence of SEQ ID NO: 720. In some cases, an antibody provided herein binds to HER3. In some embodiments, the anti-HER3 antibody comprises a heavy chain comprising an amino acid sequence that is at least 80%, at least 85%, at least 90%, at least 95%, at least 98%, or 99% identical to the amino acid sequence of SEQ ID NO: 721 and a light chain comprising an amino acid sequence that is at least 80% at least 85%, at least 90%, at least 95%, at least 98%, or 99% identical to the amino acid sequence of SEQ ID NO: 722. In some cases, an antibody provided herein binds to HER3. In some embodiments, the anti-HER3 antibody comprises a heavy chain comprising an amino acid sequence that is at least 80%, at least 85%, at least 90%, at least 95%, at least 98%, or 99% identical to the amino acid sequence of SEQ ID NO: 723 and a light chain comprising an amino acid sequence that is at least 80% at least 85%, at least 90%, at least 95%, at least 98%, or 99% identical to the amino acid sequence of SEQ ID NO: 724.

In some cases, an antibody provided herein binds to PTK7. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 725, 726, 727, 728, 729, and 730, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 725, 726, 727, 728, 729, and 730, respectively. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 733, 734, 735, 736, 737, and 738, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 733, 734, 735, 736, 737, and 738, respectively. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 741, 742, 743, 744, 745, and 746, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 741, 742, 743, 744, 745, and 746, respectively. In some cases, the anti-PTK7 antibody comprises a heavy chain comprising an amino acid sequence that is at least 80%, at least 85%, at least 90%, at least 95%, at least 98%, or 99% identical to the amino acid sequence of SEQ ID NO: 731 and a light chain comprising an amino acid sequence that is at least 80% at least 85%, at least 90%, at least 95%, at least 98%, or 99% identical to the amino acid sequence of SEQ ID NO: 732. In some cases, the anti-PTK7 antibody comprises a heavy chain comprising an amino acid sequence that is at least 80%, at least 85%, at least 90%, at least 95%, at least 98%, or 99% identical to the amino acid sequence of SEQ ID NO: 739 and a light chain comprising an amino acid sequence that is at least 80% at least 85%, at least 90%, at least 95%, at least 98%, or 99% identical to the amino acid sequence of SEQ ID NO: 740. In some cases, the anti-PTK7 antibody comprises a heavy chain comprising an amino acid sequence that is at least 80%, at least 85%, at least 90%, at least 95%, at least 98%, or 99% identical to the amino acid sequence of SEQ ID NO: 747 and a light chain comprising an amino acid sequence that is at least 80% at least 85%, at least 90%, at least 95%, at least 98%, or 99% identical to the amino acid sequence of SEQ ID NO: 748.

In some cases, an antibody provided herein binds to ZIP6. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 749, 750, 751, 752, 753, and 754, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 749, 750, 751, 752, 753, and 754, respectively. In some cases, the anti-ZIP6 antibody comprises a heavy chain variable region comprising an amino acid sequence that is at least 80%, at least 85%, at least 90%, at least 95%, at least 98%, or 99% identical to the amino acid sequence of SEQ ID NO: 755 and a light chain variable region comprising an amino acid sequence that is at least 80% at least 85%, at least 90%, at least 95%, at least 98%, or 99% identical to the amino acid sequence of SEQ ID NO: 756. In some embodiments, the anti-ZIP6 antibody comprises a heavy chain comprising an amino acid sequence that is at least 80%, at least 85%, at least 90%, at least 95%, at least 98%, or 99% identical to the amino acid sequence of SEQ ID NO: 757 and a light chain comprising an amino acid sequence that is at least 80% at least 85%, at least 90%, at least 95%, at least 98%, or 99% identical to the amino acid sequence of SEQ ID NO: 758.

In some cases, an antibody provided herein binds to Integrin αvβ6. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 759, 760, 761, 762, 763, and 764, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 759, 760, 761, 762, 763, and 764, respectively. In some embodiments, the anti-Integrin αvβ6 antibody comprises a heavy chain variable region comprising an amino acid sequence that is at least 80%, at least 85%, at least 90%, at least 95%, at least 98%, or 99% identical to the amino acid sequence of SEQ ID NO: 765 and a light chain variable region comprising an amino acid sequence that is at least 80% at least 85%, at least 90%, at least 95%, at least 98%, or 99% identical to the amino acid sequence of SEQ ID NO: 766.

In some cases, an antibody provided herein binds to CD48. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 767, 768, 769, 770, 771, and 772, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 767, 768, 769, 770, 771, and 772, respectively. In some embodiments, the anti-CD48 antibody comprises a heavy chain variable region comprising an amino acid sequence that is at least 80%, at least 85%, at least 90%, at least 95%, at least 98%, or 99% identical to the amino acid sequence of SEQ ID NO: 773 and a light chain variable region comprising an amino acid sequence that is at least 80% at least 85%, at least 90%, at least 95%, at least 98%, or 99% identical to the amino acid sequence of SEQ ID NO: 774.

In some cases, an antibody provided herein binds to IGF-1R. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 775, 776, 777, 778, 779, and 780, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 775, 776, 777, 778, 779, and 780, respectively. In some embodiments, the anti-IGF-1R antibody comprises a heavy chain variable region comprising an amino acid sequence that is at least 80%, at least 85%, at least 90%, at least 95%, at least 98%, or 99% identical to the amino acid sequence of SEQ ID NO: 781 and a light chain variable region comprising an amino acid sequence that is at least 80% at least 85%, at least 90%, at least 95%, at least 98%, or 99% identical to the amino acid sequence of SEQ ID NO: 782.

In some cases, an antibody provided herein binds to Claudin-18.2. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 783, 784, 785, 786, 787, and 788, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 783, 784, 785, 786, 787, and 788, respectively. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 791, 792, 793, 794, 795, and 796, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 791, 792, 793, 794, 795, and 796, respectively. In some embodiments, the anti-Claudin-18.2 antibody comprises a heavy chain variable region comprising an amino acid sequence that is at least 80%, at least 85%, at least 90%, at least 95%, at least 98%, or 99% identical to the amino acid sequence of SEQ ID NO: 789 and a light chain variable region comprising an amino acid sequence that is at least 80% at least 85%, at least 90%, at least 95%, at least 98%, or 99% identical to the amino acid sequence of SEQ ID NO: 790. In some embodiments, the anti-Claudin-18.2 antibody comprises a heavy chain variable region comprising an amino acid sequence that is at least 80%, at least 85%, at least 90%, at least 95%, at least 98%, or 99% identical to the amino acid sequence of SEQ ID NO: 797 and a light chain variable region comprising an amino acid sequence that is at least 80% at least 85%, at least 90%, at least 95%, at least 98%, or 99% identical to the amino acid sequence of SEQ ID NO: 798.

In some cases, an antibody provided herein binds to Nectin-4. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 799, 800, 801, 802, 803, and 804, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 799, 800, 801, 802, 803, and 804, respectively. In some embodiments, the anti-Nectin-4 antibody comprises a heavy chain variable region comprising an amino acid sequence that is at least 80%, at least 85%, at least 90%, at least 95%, at least 98%, or 99% identical to the amino acid sequence of SEQ ID NO: 805 and a light chain variable region comprising an amino acid sequence that is at least 80% at least 85%, at least 90%, at least 95%, at least 98%, or 99% identical to the amino acid sequence of SEQ ID NO: 806.

In some cases, an antibody provided herein binds to SLTRK6. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 807, 808, 809, 810, 811, and 812, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 807, 808, 809, 810, 811, and 812, respectively. In some embodiments, the anti-SLTRK6 antibody comprises a heavy chain variable region comprising an amino acid sequence that is at least 80%, at least 85%, at least 90%, at least 95%, at least 98%, or 99% identical to the amino acid sequence of SEQ ID NO: 813 and a light chain variable region comprising an amino acid sequence that is at least 80% at least 85%, at least 90%, at least 95%, at least 98%, or 99% identical to the amino acid sequence of SEQ ID NO: 814.

In some cases, an antibody provided herein binds to CD142. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 815, 816, 817, 818, 819, and 820, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 815, 816, 817, 818, 819, and 820, respectively. In some embodiments, the anti-CD142 antibody comprises a heavy chain variable region comprising an amino acid sequence that is at least 80%, at least 85%, at least 90%, at least 95%, at least 98%, or 99% identical to the amino acid sequence of SEQ ID NO: 821 and a light chain variable region comprising an amino acid sequence that is at least 80% at least 85%, at least 90%, at least 95%, at least 98%, or 99% identical to the amino acid sequence of SEQ ID NO: 822.

In some cases, an antibody provided herein binds to Sialyl-Thomsen-nouveau antigen (STn). In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 823, 824, 825, 826, 827, and 828, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 823, 824, 825, 826, 827, and 828, respectively.

In some embodiments, the anti-STn antibody comprises a heavy chain variable region comprising an amino acid sequence that is at least 80%, at least 85%, at least 90%, at least 95%, at least 98%, or 99% identical to the amino acid sequence of SEQ ID NO: 829 and a light chain variable region comprising an amino acid sequence that is at least 80% at least 85%, at least 90%, at least 95%, at least 98%, or 99% identical to the amino acid sequence of SEQ ID NO: 830.

In some cases, an antibody provided herein binds to CD20. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 831, 832, 833, 834, 835, and 836, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 831, 832, 833, 834, 835, and 836, respectively. In some embodiments, the anti-CD20 antibody comprises a heavy chain variable region comprising an amino acid sequence that is at least 80%, at least 85%, at least 90%, at least 95%, at least 98%, or 99% identical to the amino acid sequence of SEQ ID NO: 837 and a light chain variable region comprising an amino acid sequence that is at least 80% at least 85%, at least 90%, at least 95%, at least 98%, or 99% identical to the amino acid sequence of SEQ ID NO: 838.

In some cases, an antibody provided herein binds to HER2. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 839, 840, 841, 842, 843, and 844, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 839, 840, 841, 842, 843, and 844, respectively. In some embodiments, the anti-HER2 antibody comprises a heavy chain variable region comprising an amino acid sequence that is at least 80%, at least 85%, at least 90%, at least 95%, at least 98%, or 99% identical to the amino acid sequence of SEQ ID NO: 845 and a light chain variable region comprising an amino acid sequence that is at least 80% at least 85%, at least 90%, at least 95%, at least 98%, or 99% identical to the amino acid sequence of SEQ ID NO: 846.

In some cases, an antibody provided herein binds to CD79b. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 847, 848, 849, 850, 851, and 852, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 847, 848, 849, 850, 851, and 852, respectively. In some embodiments, the anti-CD79b antibody comprises a heavy chain variable region comprising an amino acid sequence that is at least 80%, at least 85%, at least 90%, at least 95%, at least 98%, or 99% identical to the amino acid sequence of SEQ ID NO: 853 and a light chain variable region comprising an amino acid sequence that is at least 80% at least 85%, at least 90%, at least 95%, at least 98%, or 99% identical to the amino acid sequence of SEQ ID NO: 854.

In some cases, an antibody provided herein binds to NaPi2B. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 855, 856, 857, 858, 859, and 860, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 855, 856, 857, 858, 859, and 860, respectively. In some embodiments, the anti-NaPi2B antibody comprises a heavy chain variable region comprising an amino acid sequence that is at least 80%, at least 85%, at least 90%, at least 95%, at least 98%, or 99% identical to the amino acid sequence of SEQ ID NO: 861 and a light chain variable region comprising an amino acid sequence that is at least 80% at least 85%, at least 90%, at least 95%, at least 98%, or 99% identical to the amino acid sequence of SEQ ID NO: 862.

In some cases, an antibody provided herein binds to Muc16. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 863, 864, 865, 866, 867, and 868, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 863, 864, 865, 866, 867, and 868, respectively. In some embodiments, the anti-Muc16 antibody comprises a heavy chain variable region comprising an amino acid sequence that is at least 80%, at least 85%, at least 90%, at least 95%, at least 98%, or 99% identical to the amino acid sequence of SEQ ID NO: 869 and a light chain variable region comprising an amino acid sequence that is at least 80% at least 85%, at least 90%, at least 95%, at least 98%, or 99% identical to the amino acid sequence of SEQ ID NO: 870.

In some cases, an antibody provided herein binds to STEAP1. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 871, 872, 873, 874, 875, and 876, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 871, 872, 873, 874, 875, and 876, respectively. In some embodiments, the anti-STEAP1 antibody comprises a heavy chain variable region comprising an amino acid sequence that is at least 80%, at least 85%, at least 90%, at least 95%, at least 98%, or 99% identical to the amino acid sequence of SEQ ID NO: 877 and a light chain variable region comprising an amino acid sequence that is at least 80% at least 85%, at least 90%, at least 95%, at least 98%, or 99% identical to the amino acid sequence of SEQ ID NO: 878.

In some cases, an antibody provided herein binds to BCMA. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 879, 880, 881, 882, 883, and 884, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 879, 880, 881, 882, 883, and 884, respectively. In some embodiments, the anti-BCMA antibody comprises a heavy chain variable region comprising an amino acid sequence that is at least 80%, at least 85%, at least 90%, at least 95%, at least 98%, or 99% identical to the amino acid sequence of SEQ ID NO: 885 and a light chain variable region comprising an amino acid sequence that is at least 80% at least 85%, at least 90%, at least 95%, at least 98%, or 99% identical to the amino acid sequence of SEQ ID NO: 886.

In some cases, an antibody provided herein binds to c-Met. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 887, 888, 889, 890, 891, and 892, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 887, 888, 889, 890, 891, and 892, respectively. In some embodiments, the anti-c-Met antibody comprises a heavy chain variable region comprising an amino acid sequence that is at least 80%, at least 85%, at least 90%, at least 95%, at least 98%, or 99% identical to the amino acid sequence of SEQ ID NO: 893 and a light chain variable region comprising an amino acid sequence that is at least 80% at least 85%, at least 90%, at least 95%, at least 98%, or 99% identical to the amino acid sequence of SEQ ID NO: 893.

In some cases, an antibody provided herein binds to SLAMF7. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 903, 904, 905, 906, 907, and 908, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 903, 904, 905, 906, 907, and 908, respectively. In some embodiments, the anti-SLAMF7 antibody comprises a heavy chain variable region comprising an amino acid sequence that is at least 80%, at least 85%, at least 90%, at least 95%, at least 98%, or 99% identical to the amino acid sequence of SEQ ID NO: 909 and a light chain variable region comprising an amino acid sequence that is at least 80% at least 85%, at least 90%, at least 95%, at least 98%, or 99% identical to the amino acid sequence of SEQ ID NO: 910.

In some cases, an antibody provided herein binds to C4.4a. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 911, 912, 913, 914, 915, and 916, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 911, 912, 913, 914, 915, and 916, respectively. In some embodiments, the anti-C4.4a antibody comprises a heavy chain variable region comprising an amino acid sequence that is at least 80%, at least 85%, at least 90%, at least 95%, at least 98%, or 99% identical to the amino acid sequence of SEQ ID NO: 917 and a light chain variable region comprising an amino acid sequence that is at least 80% at least 85%, at least 90%, at least 95%, at least 98%, or 99% identical to the amino acid sequence of SEQ ID NO: 918.

In some cases, an antibody provided herein binds to GCC. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 919, 920, 921, 922, 923, and 924, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 919, 920, 921, 922, 923, and 924, respectively. In some embodiments, the anti-GCC antibody comprises a heavy chain variable region comprising an amino acid sequence that is at least 80%, at least 85%, at least 90%, at least 95%, at least 98%, or 99% identical to the amino acid sequence of SEQ ID NO: 925 and a light chain variable region comprising an amino acid sequence that is at least 80% at least 85%, at least 90%, at least 95%, at least 98%, or 99% identical to the amino acid sequence of SEQ ID NO: 926.

In some cases, an antibody provided herein binds to Ax1. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 927, 928, 929, 930, 931, and 932, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 927, 928, 929, 930, 931, and 932, respectively. In some embodiments, the anti-Ax1 antibody comprises a heavy chain variable region comprising an amino acid sequence that is at least 80%, at least 85%, at least 90%, at least 95%, at least 98%, or 99% identical to the amino acid sequence of SEQ ID NO: 933 and a light chain variable region comprising an amino acid sequence that is at least 80% at least 85%, at least 90%, at least 95%, at least 98%, or 99% identical to the amino acid sequence of SEQ ID NO: 933.

In some cases, an antibody provided herein binds to transmembrane glycoprotein NMB (gpNMB). In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 935, 936, 937, 938, 939, and 940, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 935, 936, 937, 938, 939, and 940, respectively. In some embodiments, the anti-gpNMB antibody comprises a heavy chain variable region comprising an amino acid sequence that is at least 80%, at least 85%, at least 90%, at least 95%, at least 98%, or 99% identical to the amino acid sequence of SEQ ID NO: 941 and a light chain variable region comprising an amino acid sequence that is at least 80% at least 85%, at least 90%, at least 95%, at least 98%, or 99% identical to the amino acid sequence of SEQ ID NO: 942. In some embodiments, the anti-gpNMB antibody comprises a heavy chain variable region comprising an amino acid sequence that is at least 80%, at least 85%, at least 90%, at least 95%, at least 98%, or 99% identical to the amino acid sequence of SEQ ID NO: 943 and a light chain variable region comprising an amino acid sequence that is at least 80% at least 85%, at least 90%, at least 95%, at least 98%, or 99% identical to the amino acid sequence of SEQ ID NO: 944.

In some cases, an antibody provided herein binds to Prolactin receptor. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 945, 946, 947, 948, 949, and 950, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 945, 946, 947, 948, 949, and 950, respectively. In some embodiments, the anti-Prolactin receptor antibody comprises a heavy chain variable region comprising an amino acid sequence that is at least 80%, at least 85%, at least 90%, at least 95%, at least 98%, or 99% identical to the amino acid sequence of SEQ ID NO: 951 and a light chain variable region comprising an amino acid sequence that is at least 80% at least 85%, at least 90%, at least 95%, at least 98%, or 99% identical to the amino acid sequence of SEQ ID NO: 952.

In some cases, an antibody provided herein binds to FGFR2. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 953, 954, 955, 956, 957, and 958, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 953, 954, 955, 956, 957, and 958, respectively. In some embodiments, the anti-FGFR2 antibody comprises a heavy chain variable region comprising an amino acid sequence that is at least 80%, at least 85%, at least 90%, at least 95%, at least 98%, or 99% identical to the amino acid sequence of SEQ ID NO: 959 and a light chain variable region comprising an amino acid sequence that is at least 80% at least 85%, at least 90%, at least 95%, at least 98%, or 99% identical to the amino acid sequence of SEQ ID NO: 960.

In some cases, an antibody provided herein binds to CDCP1. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 961, 962, 963, 964, 965, and 966, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 969, 970, 971, 972, 973, and 974, respectively. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 961, 962, 963, 964, 965, and 966, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 969, 970, 971, 972, 973, and 974, respectively. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 977, 978, 979, 980, 981, and 982, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 977, 978, 979, 980, 981, and 982, respectively. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 985, 986, 987, 988, 989, and 990, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 985, 986, 987, 988, 989, and 990, respectively. In some embodiments, the anti-CDCP1 antibody comprises a heavy chain variable region comprising an amino acid sequence that is at least 80%, at least 85%, at least 90%, at least 95%, at least 98%, or 99% identical to the amino acid sequence of SEQ ID NO: 967 and a light chain variable region comprising an amino acid sequence that is at least 80% at least 85%, at least 90%, at least 95%, at least 98%, or 99% identical to the amino acid sequence of SEQ ID NO: 968. In some embodiments, the anti-CDCP1 antibody comprises a heavy chain variable region comprising an amino acid sequence that is at least 80%, at least 85%, at least 90%, at least 95%, at least 98%, or 99% identical to the amino acid sequence of SEQ ID NO: 975 and a light chain variable region comprising an amino acid sequence that is at least 80% at least 85%, at least 90%, at least 95%, at least 98%, or 99% identical to the amino acid sequence of SEQ ID NO: 976. In some embodiments, the anti-CDCP1 antibody comprises a heavy chain variable region comprising an amino acid sequence that is at least 80%, at least 85%, at least 90%, at least 95%, at least 98%, or 99% identical to the amino acid sequence of SEQ ID NO: 983 and a light chain variable region comprising an amino acid sequence that is at least 80% at least 85%, at least 90%, at least 95%, at least 98%, or 99% identical to the amino acid sequence of SEQ ID NO: 984. In some embodiments, the anti-CDCP1 antibody comprises a heavy chain variable region comprising an amino acid sequence that is at least 80%, at least 85%, at least 90%, at least 95%, at least 98%, or 99% identical to the amino acid sequence of SEQ ID NO: 991 and a light chain variable region comprising an amino acid sequence that is at least 80% at least 85%, at least 90%, at least 95%, at least 98%, or 99% identical to the amino acid sequence of SEQ ID NO: 992.

In some cases, an antibody provided herein binds to ASCT2. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 997, 998, 999, 1000, 1001, and 1002, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 997, 998, 999, 1000, 1001, and 1002, respectively. In some embodiments, the anti-ASCT2 antibody comprises a heavy chain variable region comprising an amino acid sequence that is at least 80%, at least 85%, at least 90%, at least 95%, at least 98%, or 99% identical to the amino acid sequence of SEQ ID NO: 993 and a light chain variable region comprising an amino acid sequence that is at least 80% at least 85%, at least 90%, at least 95%, at least 98%, or 99% identical to the amino acid sequence of SEQ ID NO: 994. In some embodiments, the anti-ASCT2 antibody comprises a heavy chain variable region comprising an amino acid sequence that is at least 80%, at least 85%, at least 90%, at least 95%, at least 98%, or 99% identical to the amino acid sequence of SEQ ID NO: 995 and a light chain variable region comprising an amino acid sequence that is at least 80% at least 85%, at least 90%, at least 95%, at least 98%, or 99% identical to the amino acid sequence of SEQ ID NO: 996. In some embodiments, the anti-ASCT2 antibody comprises a heavy chain variable region comprising an amino acid sequence that is at least 80%, at least 85%, at least 90%, at least 95%, at least 98%, or 99% identical to the amino acid sequence of SEQ ID NO: 1003 and a light chain variable region comprising an amino acid sequence that is at least 80% at least 85%, at least 90%, at least 95%, at least 98%, or 99% identical to the amino acid sequence of SEQ ID NO: 1004.

In some cases, an antibody provided herein binds to CD123. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 1005, 1006, 1007, 1008, 1009, and 1010, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 1005, 1006, 1007, 1008, 1009, and 1010, respectively. In some embodiments, the anti-CD123 antibody comprises a heavy chain variable region comprising an amino acid sequence that is at least 80%, at least 85%, at least 90%, at least 95%, at least 98%, or 99% identical to the amino acid sequence of SEQ ID NO: 1011 and a light chain variable region comprising an amino acid sequence that is at least 80% at least 85%, at least 90%, at least 95%, at least 98%, or 99% identical to the amino acid sequence of SEQ ID NO: 1012.

In some cases, an antibody provided herein binds to GPC3. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 1013, 1014, 1015, 1016, 1017, and 1018 respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 1013, 1014, 1015, 1016, 1017, and 1018, respectively. In some embodiments, the anti-TIGIT antibody comprises a heavy chain variable region comprising an amino acid sequence that is at least 80%, at least 85%, at least 90%, at least 95%, at least 98%, or 99% identical to the amino acid sequence of SEQ ID NO: 1027 and a light chain variable region comprising an amino acid sequence that is at least 80% at least 85%, at least 90%, at least 95%, at least 98%, or 99% identical to the amino acid sequence of SEQ ID NO: 1028.

In some cases, an antibody provided herein binds to TIGIT. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 1021, 1022, 1023, 1024, 1025, and 1026, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 1021, 1022, 1023, 1024, 1025, and 1026, respectively. In some embodiments, the anti-BCMA antibody comprises a heavy chain variable region comprising an amino acid sequence that is at least 80%, at least 85%, at least 90%, at least 95%, at least 98%, or 99% identical to the amino acid sequence of SEQ ID NO: 1043 and a light chain variable region comprising an amino acid sequence that is at least 80% at least 85%, at least 90%, at least 95%, at least 98%, or 99% identical to the amino acid sequence of SEQ ID NO: 1044.

In some cases, an antibody provided herein binds to CD33. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 1029, 1030, 1031, 1032, 1033, and 1034, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 1029, 1030, 1031, 1032, 1033, and 1034, respectively. In some embodiments, the anti-CD33 antibody comprises a heavy chain variable region comprising an amino acid sequence that is at least 80%, at least 85%, at least 90%, at least 95%, at least 98%, or 99% identical to the amino acid sequence of SEQ ID NO: 1035 and a light chain variable region comprising an amino acid sequence that is at least 80% at least 85%, at least 90%, at least 95%, at least 98%, or 99% identical to the amino acid sequence of SEQ ID NO: 1036.

In some cases, an antibody provided herein binds to BCMA. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 1037, 1038, 1039, 1040, 1041, and 1042, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 1037, 1038, 1039, 1040, 1041, and 1042, respectively. In some embodiments, the anti-BCMA antibody comprises a heavy chain variable region comprising an amino acid sequence that is at least 80%, at least 85%, at least 90%, at least 95%, at least 98%, or 99% identical to the amino acid sequence of SEQ ID NO: 1043 and a light chain variable region comprising an amino acid sequence that is at least 80% at least 85%, at least 90%, at least 95%, at least 98%, or 99% identical to the amino acid sequence of SEQ ID NO: 1044.

### Ligand-Drug Conjugate Compounds of Compound 3.1

In some embodiments, provided is a compound 3.1,

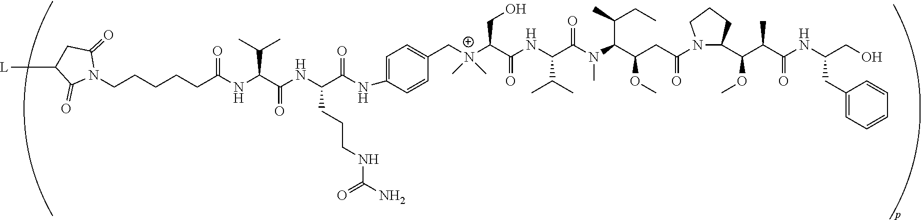

wherein L is a ligand (e.g., an antibody) that targets an antigen selected from the group consisting of ADAM12 (e.g., Catalog #14139-1-AP); ADAM9 (e.g., IMGC936); AFP (e.g., ThermoFisher Catalog #PA5-25959); AGR2 (e.g., ThermoFisher Catalog #PA5-34517); AKAP-4 (e.g., Catalog #PA5-52230); ALK (e.g., DLX521); ALPP (e.g., Catalog #MA5-15652); ALPPL2 (e.g., Catalog #PA5-22336); AMHR2 (e.g., ThermoFisher Catalog #PA5-13902); androgen receptor (e.g., ThermoFisher Catalog #MA5-13426); ANTXR1 (e.g., Catalog #MA1-91702); ANXA1 (e.g., Catalog #71-3400); ARTN (e.g., ThermoFisher Catalog #PA5-47063); ASCT2 (e.g., idactamab); Ax1 (e.g., BA3011; tilvestamab); B7-DC (e.g., Catalog #PA5-20344); B7-H3 (e.g., enoblituzumab, omburtamab, MGD009, MGC018, DS-7300); B7-H4 (e.g., Catalog #14-5949-82); B7-H6 (e.g., Catalog #12-6526-42); B7-H7; BAFF-R (e.g., Catalog #14-9117-82); BCMA; BCR-ABL; BMPR2; BORIS; C4.4a; CanAg; C5 complement (e.g., BCD-148; CAN106); CA-125; CA19-9 (e.g., AbGn-7; MVT-5873); CA9 (e.g., girentuximab); CALCR (see, e.g., International Publication No. WO 2015077826); CAMPATH-1 (e.g., alemtuzumab; ALLO-647; ANT1034); carcinoembryonic antigen (e.g., arcitumomab; cergutuzumab; amunaleukin; labetuzumab); CCNB1; CD112 (see, e.g., U.S. Publication No. 20100008928); CD115 (e.g., axatilimab; cabiralizumab; emactuzumab); CD123 (e.g., BAY-943; CSL360); CD137 (e.g., ADG106; CTX-471); CD138; CD142; CD166; CD147 (e.g., gavilimomab; metuzumab); CD155 (e.g., U.S. Publication No. 2018/0251548); CD19 (e.g., ALLO-501); CD20 (e.g., divozilimab; ibritumomab tiuxetan); CD24 (see, e.g., U.S. Pat. No. 8,614,301); CD244 (e.g., R&D AF1039); CD247 (e.g., AFM15); CD27 (e.g., varlilumab); CD274 (e.g., adebrelimab; atezolizumab; garivulimab); CD3 (e.g., otelixizumab; visilizumab); CD30 (e.g., iratumumab); CD33 (e.g., lintuzumab; BI 836858; AMG 673); CD288; CD352 (e.g., SGN-CD352A); CD37 (e.g., lilotomab; GEN3009); CD38 (e.g., felzartamab; AMG 424); CD3D; CD3E (e.g., foralumab; teplizumab); CD3G; CD45 (e.g., apamistamab); CD47 (e.g., letaplimab; magrolimab); CD48 (e.g., SGN-CD48A); CD5 (e.g., MAT 304; zolimomab aritox); CD56; CD59; CD70 (e.g., cusatuzumab); CD74 (e.g., milatuzumab); CD79A (see, e.g., International Publication No. WO 2020252110); CD79b; CD96; CD97; CD-262 (e.g., tigatuzumab); CDCP1 (e.g., RG7287); CDH17 (see, e.g., International Publication No. WO 2018115231); CDH3 (e.g., PCA062); CDH6 (e.g., HKT288); CEACAMI; CEACAM5; CEACAM6; CLDN1 (e.g., INSERM anti-Claudin-1); CLDN16; CLDN18.1 (e.g., zolbetuximab); CLDN18.2 (e.g., zolbetuximab); CLDN19; CLDN2 (see, e.g., International Publication No. WO 2018123949); CLEC12A (e.g., tepoditamab); CS1; CLPTM1L; CSPG4 (e.g., U.S. Pat. No. 10,822,427); CXCR4 (e.g., ulocuplumab); CYP1B1; c-Met; DCLK1 (see, e.g., International Publication No. WO 2018222675); DDR1; de2-7 EGFR (e.g., MAb 806); DLL-3; DPEP1; DPEP3; DPP4; DR4 (e.g., mapatumumab); DSG2 (see, e.g., U.S. Pat. No. 10,836,823); EGF; EGFR; endosialin (e.g., ontuxizumab); ENPP1; EPCAM (e.g., adecatumumab); EPHA receptors; EPHA2; ERBB2 (e.g., trastuzumab); ERBB3; ERVMER34_1; ETV6-AML (e.g., Catalog #PA5-81865); FAS; FasL; Fas-related antigen 1; FBP; FGFR1 (e.g., RG7992); FGFR2 (e.g., aprutumab); FGFR3 (e.g., vofatamab); FGFR4 (e.g., MM-161); FLT3 (e.g., 4G8SDIEM); FN; FN1; FOLR1 (e.g., farletuzumab); FRa; FSHR; FucGM1 (e.g., BMS-986012); FZD5; FZD8; G250; GAGE; GCC; GD2 (e.g., dinutuximab); GD3 (e.g., mitumomab); GITR (e.g., ragifilimab); GloboH; GM2 (e.g., BIW-8962); GM3 (e.g., racotumomab); gp100; GPA33 (e.g., KRN330); GPC3 (e.g., codrituzumab); gpNMB (e.g., glembatumumab); GPR87; GUCY2C (e.g., indusatumab); HAS3; HAVCR2; HLA-E; HLA-F; HLA-G (e.g., TTX-080); HPV E6 E7; hTERT; ICAM1; IDO1; IFNAR1 (e.g., faralimomab); IFNAR2; IL13Ra2; ILIRAP (e.g., nidanilimab); IL-21R (e.g., PF-05230900); IL-5R (e.g., benralizumab); ITGAV (e.g., abituzumab); ITGB6; ITGB8; KISSIR; LICAM (e.g., JCAR023); LAG-3 (e.g., encelimab); LAMP1; LCK; legumain; LMP2; LY6G6D (e.g., PA5-23303); LY9 (e.g., PA5-95601); LYPD1 (e.g., ThermoFisher Catalog #PA5-26749); MAD-CT-1; MAD-CT-2; MAGEA1 (e.g., Catalog #MA5-11338); MAGEA3 (e.g., ThermoFisher Catalog #60054-1-IG); MAGEA4 (e.g., Catalog #MA5-26117); MAGEC2 (e.g., ThermoFisher Catalog #PA5-64010); MELTF (e.g., ThermoFisher Catalog #H00004241-M04A); MerTk (e.g., DS5MMER, Catalog #12-5751-82); a metalloproteinase; MFSDi3A; MICA (e.g., 1E2C8, Catalog #66384-1-IG); MICB (e.g., Catalog #MA5-29422); Mincle (e.g., OTI2A8, Catalog #TA505101); MLANA (e.g., Catalog #MA5-15237); ML-IAP (e.g., 88C570, ThermoFisher Catalog #40958); MSLN (e.g., 5B2, Catalog #MA5-11918); MUC1 (e.g., MHI (CT2), ThermoFisher Catalog #MA5-11202); MUC5AC (e.g., 45M1, Catalog #MA5-12178); MYCN (e.g., NCM-II 100, ThermoFisher Catalog #MA1-170); NA17; NCAM1 (e.g., ThermoFisher Catalog #MA5-11563); Nectin-4 (e.g., enfortumab); NOX1 (e.g., Catalog #PA5-103220); NT5E (e.g., 7G2, ThermoFisher Catalog #41-0200); NY-BR-I (e.g., NY-BR-I No. 2, Catalog #MA5-12645); NY-ESO-I (e.g., E978m, Catalog #35-6200); OX40 (e.g., ABM193); OY-TESI; p53; p53mutant; PAP; PAX3 (e.g., GT1210, ThermoFisher Catalog #MA5-31583); PAX5; PDGFR-B (e.g., rinucumab); PDPN (e.g., ThermoFisher Catalog #14-5381-82); PLAVl; PMSA; polysialic acid (see, e.g., Watzlawik et al. J Nat Sci. 2015; 1(8):e141); PRI; PROMI (e.g., Catalog #14-1331-82); PSA (e.g., ThermoFisher Catalog #PA1-38514; Daniels-Wells et al. BMC Cancer 2013; 13:195); PSCA (e.g., AGS-1C4D4); PSMA (e.g., BAY 2315497); PTK7 (e.g., cofetuzumab); PVRIG; Ras mutant (e.g., Shin et al. Sci Adv. 2020; 6(3):eaay2174); RET (e.g., WO2020210551); RGS5 (e.g., TF-TA503075); RhoC (e.g., ThermoFisher Catalog PA5-77866); ROR1 (e.g., cirmtuzumab); ROR2 (e.g., BA3021); ROSI (e.g., WO 2019107671); Sarcoma translocation breakpoints; SART3 (e.g., TF 18025-1-AP); Sialyl-Thomsen-nouveau-antigen (e.g., Eavarone et al. PLoS One. 2018; 13(7): e0201314); Siglecs 1-16 (see, e.g., Angata et al. Trends Pharmacol Sci. 2015; 36(10): 645-660); SIRPa (e.g., Catalog #17-1729-42); SIRPg (e.g., PA5-104381); SIT1 (e.g., PA5-53825); SLAMF7 (e.g., elotuzumab); SLCIOA2 (e.g., ThermoFisher Catalog #PA5-18990); SLC12A2 (e.g., ThermoFisher Catalog #13884-1-AP); SLC17A2 (e.g., ThermoFisher Catalog #PA5-106752); SLC38A1 (e.g., ThermoFisher Catalog #12039-1-AP); SLC39A5 (e.g., ThermoFisher Catalog #MA5-27260); SLC39A6 (e.g., ladiratuzumab); SLC44A4 (e.g., ASG-5ME); SLC6A15 (e.g., ThermoFisher Catalog #PA5-52586); SLC6A6 (e.g., ThermoFisher Catalog #PA5-53431); SLC7A11 (e.g., ThermoFisher Catalog #PA1-16893); SLC7A5; sLe; SLITRK6 (e.g., sirtratumab); Sperm protein 17 (e.g., BS-5754R); SSX2 (e.g., ThermoFisher Catalog #MA5-24971); survivin (e.g., PA1-16836); TACSTD2 (e.g., PA5-47074); TAG-72 (e.g., MA1-25956); tenascin; TF (e.g., tisotumab); Tie3; TLR2/4/1 (e.g., tomaralimab); TM4SF5 (e.g., 18239-1-AP); TMEM132A (e.g., Catalog #PA5-62524); TMEM40 (e.g., PA5-60636); TMPRSS11D (e.g., PA5-30927); Tn; TNFRSF12 (e.g., BAY-356); TRAIL (e.g., Catalog #12-9927-42); TRAIL1; TRP-2 (e.g., PA5-52736); ULBP1/2/3/4/5/6 (e.g., PA5-82302); uPAR (e.g., ATN-658); UPK1B (e.g., ThermoFisher Catalog #PA5-56863); UPK2 (e.g., ThermoFisher Catalog #PA5-60318); UPK3B (e.g., ThermoFisher Catalog #PA5-52696); VEGF (e.g., GNR-011); VEGFR2 (e.g., gentuximab); VSIR (e.g., ThermoFisher Catalog #PA5-52493); WT1 (e.g., ThermoFisher Catalog #MA5-32215); and XAGE1 (e.g., ThermoFisher Catalog #PA5-46413).

Also provided herein is a compound 3.1, wherein L is a ligand (e.g., an antibody) that targets an antigen selected from the group consisting of Ax1 (e.g., BA3011; tilvestamab); B7-1 (e.g., galiximab); B7-2 (e.g., Catalog #12-0862-82); B7-DC (e.g., Catalog #PA5-20344); B7-H3 (e.g., enoblituzumab, omburtamab, MGD009, MGC018, DS-7300); B7-H4 (e.g., Catalog #14-5949-82); B7-H6 (e.g., Catalog #12-6526-42); B7-H7; BAFF-R (e.g., Catalog #14-9117-82); BCMA; C5 complement (e.g., BCD-148; CAN106); CCR4 (e.g., AT008; mogamulizumab-kpkc); CCR8 (e.g., JTX-1811); CD112 (see, e.g., U.S. Publication No. 20100008928); CD115 (e.g., axatilimab; cabiralizumab; emactuzumab); CD123 (e.g., BAY-943; CSL360); CD137 (e.g., ADG106; CTX-471); CD155 (e.g., U.S. Publication No. 2018/0251548); CD163 (e.g., TBI 304H); CD19 (e.g., ALLO-501); CD2 (e.g., BTI-322; siplizumab); CD20 (e.g., divozilimab; ibritumomab); CD24 (see, e.g., U.S. Pat. No. 8,614,301); CD244 (e.g., R&D AF1039); CD247 (e.g., AFM15); CD25 (e.g., basiliximab); CD27 (e.g., varlilumab); CD274 (e.g., adebrelimab; atezolizumab; garivulimab); CD278 (e.g., feladilimab; vopratelimab); CD28 (e.g., REGN5668); CD3 (e.g., otelixizumab; visilizumab); CD30 (e.g., iratumumab); CD30L (see, e.g., U.S. Pat. No. 9,926,373); CD32 (e.g., mAb 2B6); CD33 (e.g., lintuzumab; BI 836858; AMG 673); CD352 (e.g., SGN-CD352A); CD37 (e.g., lilotomab; GEN3009); CD38 (e.g., felzartamab; AMG 424); CD3D; CD3E (e.g., foralumab; teplizumab); CD3G; CD40 (e.g., dacetuzumab; lucatumumab); CD44 (e.g., RG7356); CD45 (e.g., apamistamab); CD47 (e.g., letaplimab; magrolimab); CD48 (e.g., SGN-CD48A); CD5 (e.g., MAT 304; zolimomab aritox); CD51; CD70 (e.g., cusatuzumab); CD74 (e.g., milatuzumab); CD79A (see, e.g., International Publication No. WO 2020252110); CD83 (e.g., CBT004); CD97; CD262 (e.g., tigatuzumab); CLEC12A (e.g., tepoditamab); CTLA4 (e.g., ipilimumab); CXCR4 (e.g., ulocuplumab); DCIR; DCSIGN (see, e.g., International Publication No. WO 2018134389); Dectin1 (see, e.g., U.S. Pat. No. 9,045,542); Dectin2 (e.g., ThermoFisher Catalog #MA5-16250); DR4 (e.g., mapatumumab); endosialin (e.g., ontuxizumab); FasL; FLT3 (e.g., 4G8SDIEM); GITR (e.g., ragifilimab); HAVCR2; HER2; HER3; HLA-DR; HLA-E; HLA-F; HLA-G (e.g., TTX-080); ICAM1; IDO1; IFNAR1 (e.g., faralimomab); IFNAR2; IGF-1R; ILIRAP (e.g., nidanilimab); IL-21R (e.g., PF-05230900); IL-5R (e.g., benralizumab); Integrin αvβ6; LAG-3 (e.g., encelimab); LAMP1; LAYN; LCK; LILRB2; LILRB4; MerTk (e.g., DS5MMER, Catalog #12-5751-82); Mesothelin; MICA (e.g., 1E2C8, Catalog #66384-1-IG); MICB (e.g., Catalog #MA5-29422); MICA; Mincle (e.g., OTI2A8, Catalog #TA505101); MRC1 (e.g., ThermoFisher Catalog #12-2061-82); MUC1; Muc16; NcaPi2B; Nectin-4; OX40 (e.g., ABM193); PD-1 (e.g., balstilimab; budigalimab; geptanolimab); PD-L1; Prolactin receptor; PTK7; PVRIG; ROR-1; Sialyl-Thomsen-nouveau-antigen (e.g., Eavarone et al. PLoS One, 2018; 13(7): e0201314); Siglecs 1-16 (see, e.g., Angata et al. Trends Pharmacol Sci. 2015; 36(10): 645-660); SIRPa (e.g., Catalog #17-1729-42); SIRPg (e.g., PA5-104381); SIT1 (e.g., PA5-53825); SLAMF7 (e.g., elotuzumab); SLTRK6; STEAP1; TIGIT (e.g., etigilimab); TLR2/4/1 (e.g., tomaralimab); Trem2 (e.g., PY314); TROP2; Tyrol; ULBP1/2/3/4/5/6 (e.g., PA5-82302); uPAR (e.g., ATN-658); VSIR (e.g., ThermoFisher Catalog #PA5-52493); and ZIP6 (Anti-Integrin αvβ6).

(v) Heavy Chain and Light Chain Variable Regions is a Compound 3.1

In some embodiments, the L moiety of compound 3.1 is a ligand (e.g., an antibody or fragment thereof) that targets ADAM9, ASCT2, Ax1, B7-H3, B7H4, BCMA, BCMA, C4.4a, CanAg, CD123, CD138, CD142, CD166, CD19, CD20, CD228, CD25, CD30, CD33, CD352, CD38, CD48, CD56, CD59, CD70, CD74, CD79b, CDCP1, CEACAM5, Claudin-18.2, c-Met, gpNMB, CS1, DLL-3, DPEP-3, EGFR, EpCAM, EphA2, FGFR2, FRa, GCC, gpA33, GPC3, Integrin αvβ6, h2A2, H2G12/STn, HER2, HER3, ZIP6, IGF-1R, IL1Rap, ITGav/CD51, Mesothelin, MICA, MUC-1, Muc16, NaPi2B, Nectin-4, PD-L1, Prolactin receptor, PTK7, ROR-1, SLAMF7, SLTRK6, STEAP1, TIGIT, or TROP2.

In some embodiments, the L moiety of compound 3.1 comprises a heavy chain variable region having at least 80% sequence identity to a first sequence and a light chain variable region having at least 80% sequence identity to a second sequence, and wherein: the first sequence is SEQ ID NO: 19 and the second sequence is SEQ ID NO: 20; the first sequence is SEQ ID NO: 44 and the second sequence is SEQ ID NO: 45; the first sequence is SEQ ID NO: 55 and the second sequence is SEQ ID NO: 56; the first sequence is SEQ ID NO: 69 and the second sequence is SEQ ID NO: 70; the first sequence is SEQ ID NO: 83 and the second sequence is SEQ ID NO: 84; the first sequence is SEQ ID NO: 97 and the second sequence is SEQ ID NO: 98; the first sequence is SEQ ID NO: 105 and the second sequence is SEQ ID NO: 106; the first sequence is SEQ ID NO:113 and the second sequence is SEQ ID NO: 114; the first sequence is SEQ ID NO: 121 and the second sequence is SEQ ID NO: 122; the first sequence is SEQ ID NO: 129 and the second sequence is SEQ ID NO: 130; the first sequence is SEQ ID NO: 137 and the second sequence is SEQ ID NO: 138; the first sequence is SEQ ID NO: 153 and the second sequence is SEQ ID NO: 154; the first sequence is SEQ ID NO: 161 and the second sequence is SEQ ID NO: 162; the first sequence is SEQ ID NO: 169 and the second sequence is SEQ ID NO: 170; the first sequence is SEQ ID NO: 177 and the second sequence is SEQ ID NO: 178; the first sequence is SEQ ID NO: 185 and the second sequence is SEQ ID NO: 186; the first sequence is SEQ ID NO: 193 and the second sequence is SEQ ID NO: 194; the first sequence is SEQ ID NO: 201 and the second sequence is SEQ ID NO: 202; the first sequence is SEQ ID NO: 209 and the second sequence is SEQ ID NO: 210; the first sequence is SEQ ID NO: 217 and the second sequence is SEQ ID NO: 218; the first sequence is SEQ ID NO: 225 and the second sequence is SEQ ID NO: 226; the first sequence is SEQ ID NO: 233 and the second sequence is SEQ ID NO: 234; the first sequence is SEQ ID NO: 241 and the second sequence is SEQ ID NO: 242; the first sequence is SEQ ID NO: 249 and the second sequence is SEQ ID NO: 250; the first sequence is SEQ ID NO: 297 and the second sequence is SEQ ID NO: 298; the first sequence is SEQ ID NO: 307 and the second sequence is SEQ ID NO: 308; the first sequence is SEQ ID NO: 315 and the second sequence is SEQ ID NO: 316; the first sequence is SEQ ID NO: 323 and the second sequence is SEQ ID NO: 324; the first sequence is SEQ ID NO: 331 and the second sequence is SEQ ID NO: 332; the first sequence is SEQ ID NO: 339 and the second sequence is SEQ ID NO: 340; the first sequence is SEQ ID NO: 347 and the second sequence is SEQ ID NO: 348; the first sequence is SEQ ID NO: 355 and the second sequence is SEQ ID NO: 356; the first sequence is SEQ ID NO: 363 and the second sequence is SEQ ID NO: 364; the first sequence is SEQ ID NO: 371 and the second sequence is SEQ ID NO: 372; the first sequence is SEQ ID NO: 379 and the second sequence is SEQ ID NO: 380; the first sequence is SEQ ID NO: 387 and the second sequence is SEQ ID NO: 388; the first sequence is SEQ ID NO: 395 and the second sequence is SEQ ID NO: 396; the first sequence is SEQ ID NO: 403 and the second sequence is SEQ ID NO: 404; the first sequence is SEQ ID NO: 411 and the second sequence is SEQ ID NO: 412; the first sequence is SEQ ID NO: 419 and the second sequence is SEQ ID NO: 420; the first sequence is SEQ ID NO: 427 and the second sequence is SEQ ID NO: 428; the first sequence is SEQ ID NO: 435 and the second sequence is SEQ ID NO: 436; the first sequence is SEQ ID NO: 443 and the second sequence is SEQ ID NO: 444; the first sequence is SEQ ID NO: 451 and the second sequence is SEQ ID NO: 452; the first sequence is SEQ ID NO: 459 and the second sequence is SEQ ID NO: 460; the first sequence is SEQ ID NO: 467 and the second sequence is SEQ ID NO: 468; the first sequence is SEQ ID NO: 475 and the second sequence is SEQ ID NO: 476; the first sequence is SEQ ID NO: 483 and the second sequence is SEQ ID NO: 484; the first sequence is SEQ ID NO: 491 and the second sequence is SEQ ID NO: 492; the first sequence is SEQ ID NO: 501 and the second sequence is SEQ ID NO: 502; the first sequence is SEQ ID NO: 509 and the second sequence is SEQ ID NO: 510; the first sequence is SEQ ID NO: 517 and the second sequence is SEQ ID NO: 518; the first sequence is SEQ ID NO: 525 and the second sequence is SEQ ID NO: 526; the first sequence is SEQ ID NO: 533 and the second sequence is SEQ ID NO: 534; the first sequence is SEQ ID NO: 541 and the second sequence is SEQ ID NO: 542; the first sequence is SEQ ID NO: 549 and the second sequence is SEQ ID NO: 550; the first sequence is SEQ ID NO: 557 and the second sequence is SEQ ID NO: 558; the first sequence is SEQ ID NO: 565 and the second sequence is SEQ ID NO: 566; the first sequence is SEQ ID NO: 574 and the second sequence is SEQ ID NO: 574; the first sequence is SEQ ID NO: 581 and the second sequence is SEQ ID NO: 582; the first sequence is SEQ ID NO: 589 and the second sequence is SEQ ID NO: 590; the first sequence is SEQ ID NO: 597 and the second sequence is SEQ ID NO: 598; the first sequence is SEQ ID NO: 605 and the second sequence is SEQ ID NO: 606; the first sequence is SEQ ID NO: 613 and the second sequence is SEQ ID NO: 614; the first sequence is SEQ ID NO: 621 and the second sequence is SEQ ID NO: 622; the first sequence is SEQ ID NO: 629 and the second sequence is SEQ ID NO: 630; the first sequence is SEQ ID NO: 637 and the second sequence is SEQ ID NO: 638; the first sequence is SEQ ID NO: 645 and the second sequence is SEQ ID NO: 646; the first sequence is SEQ ID NO: 653 and the second sequence is SEQ ID NO: 654; the first sequence is SEQ ID NO: 661 and the second sequence is SEQ ID NO: 662; the first sequence is SEQ ID NO: 669 and the second sequence is SEQ ID NO: 670; the first sequence is SEQ ID NO: 677 and the second sequence is SEQ ID NO: 678; the first sequence is SEQ ID NO: 685 and the second sequence is SEQ ID NO: 686; the first sequence is SEQ ID NO: 693 and the second sequence is SEQ ID NO: 694; the first sequence is SEQ ID NO: 701 and the second sequence is SEQ ID NO: 702; the first sequence is SEQ ID NO: 703 and the second sequence is SEQ ID NO: 704; the first sequence is SEQ ID NO: 711 and the second sequence is SEQ ID NO: 712; the first sequence is SEQ ID NO: 713 and the second sequence is SEQ ID NO: 714; the first sequence is SEQ ID NO: 715 and the second sequence is SEQ ID NO: 716; the first sequence is SEQ ID NO: 731 and the second sequence is SEQ ID NO: 732; the first sequence is SEQ ID NO: 739 and the second sequence is SEQ ID NO: 740; the first sequence is SEQ ID NO: 747 and the second sequence is SEQ ID NO: 748; the first sequence is SEQ ID NO: 755 and the second sequence is SEQ ID NO: 756; the first sequence is SEQ ID NO: 765 and the second sequence is SEQ ID NO: 766; the first sequence is SEQ ID NO: 773 and the second sequence is SEQ ID NO: 774; the first sequence is SEQ ID NO: 781 and the second sequence is SEQ ID NO: 782; the first sequence is SEQ ID NO: 789 and the second sequence is SEQ ID NO: 790; the first sequence is SEQ ID NO: 797 and the second sequence is SEQ ID NO: 798; the first sequence is SEQ ID NO: 805 and the second sequence is SEQ ID NO: 806; the first sequence is SEQ ID NO: 813 and the second sequence is SEQ ID NO: 814; the first sequence is SEQ ID NO: 821 and the second sequence is SEQ ID NO: 822; the first sequence is SEQ ID NO: 829 and the second sequence is SEQ ID NO: 830; the first sequence is SEQ ID NO: 837 and the second sequence is SEQ ID NO: 838; the first sequence is SEQ ID NO: 845 and the second sequence is SEQ ID NO: 846; the first sequence is SEQ ID NO: 853 and the second sequence is SEQ ID NO: 854; the first sequence is SEQ ID NO: 861 and the second sequence is SEQ ID NO: 862; the first sequence is SEQ ID NO: 869 and the second sequence is SEQ ID NO: 870; the first sequence is SEQ ID NO: 877 and the second sequence is SEQ ID NO: 878; the first sequence is SEQ ID NO: 885 and the second sequence is SEQ ID NO: 886; the first sequence is SEQ ID NO: 893 and the second sequence is SEQ ID NO: 894; the first sequence is SEQ ID NO: 900 and the second sequence is SEQ ID NO: 901; the first sequence is SEQ ID NO: 909 and the second sequence is SEQ ID NO: 910; the first sequence is SEQ ID NO: 917 and the second sequence is SEQ ID NO: 918; the first sequence is SEQ ID NO: 925 and the second sequence is SEQ ID NO: 926; the first sequence is SEQ ID NO: 933 and the second sequence is SEQ ID NO: 934; the first sequence is SEQ ID NO: 941 and the second sequence is SEQ ID NO: 942; the first sequence is SEQ ID NO: 943 and the second sequence is SEQ ID NO: 944; the first sequence is SEQ ID NO: 951 and the second sequence is SEQ ID NO: 952; the first sequence is SEQ ID NO: 959 and the second sequence is SEQ ID NO: 960; the first sequence is SEQ ID NO: 967 and the second sequence is SEQ ID NO: 968; the first sequence is SEQ ID NO: 975 and the second sequence is SEQ ID NO: 976; the first sequence is SEQ ID NO: 983 and the second sequence is SEQ ID NO: 984; the first sequence is SEQ ID NO: 991 and the second sequence is SEQ ID NO: 992; the first sequence is SEQ ID NO: 993 and the second sequence is SEQ ID NO: 994; the first sequence is SEQ ID NO: 995 and the second sequence is SEQ ID NO: 996; the first sequence is SEQ ID NO: 1003 and the second sequence is SEQ ID NO: 1004; the first sequence is SEQ ID NO: 1011 and the second sequence is SEQ ID NO: 1012; the first sequence is SEQ ID NO: 1019 and the second sequence is SEQ ID NO: 1020; the first sequence is SEQ ID NO: 1027 and the second sequence is SEQ ID NO: 1028; the first sequence is SEQ ID NO: 1035 and 1036; or the first sequence is SEQ ID NO: 1043 and the second sequence is SEQ ID NO: 1044.

In some embodiments, the L moiety of compound 3.1 comprises an antibody that targets an immune checkpoint selected from the group consisting of PDL1, B7H4, B7H3, and TIGIT. For example, in some cases, the antibody comprises a heavy chain variable region having at least 80% sequence identity to a first sequence and a light chain variable region having at least 80% sequence identity to a second sequence, wherein: the first sequence is SEQ ID NO: 19 and the second sequence is SEQ ID NO: 20; the first sequence is SEQ ID NO: 83 and the second sequence is SEQ ID NO: 84; the first sequence is SEQ ID NO: 97 and the second sequence is SEQ ID NO: 98; the first sequence is SEQ ID NO: 105 and the second sequence is SEQ ID NO: 106; the first sequence is SEQ ID NO:113 and the second sequence is SEQ ID NO: 114; the first sequence is SEQ ID NO: 121 and the second sequence is SEQ ID NO: 122; the first sequence is SEQ ID NO: 129 and the second sequence is SEQ ID NO: 130; the first sequence is SEQ ID NO: 137 and the second sequence is SEQ ID NO: 138; the first sequence is SEQ ID NO: 153 and the second sequence is SEQ ID NO: 154; the first sequence is SEQ ID NO: 161 and the second sequence is SEQ ID NO: 162; the first sequence is SEQ ID NO: 169 and the second sequence is SEQ ID NO: 170; the first sequence is SEQ ID NO: 177 and the second sequence is SEQ ID NO: 178; the first sequence is SEQ ID NO: 185 and the second sequence is SEQ ID NO: 186; the first sequence is SEQ ID NO: 193 and the second sequence is SEQ ID NO: 194; the first sequence is SEQ ID NO: 201 and the second sequence is SEQ ID NO: 202; the first sequence is SEQ ID NO: 209 and the second sequence is SEQ ID NO: 210; the first sequence is SEQ ID NO: 217 and the second sequence is SEQ ID NO: 218; the first sequence is SEQ ID NO: 225 and the second sequence is SEQ ID NO: 226; the first sequence is SEQ ID NO: 233 and the second sequence is SEQ ID NO: 234; the first sequence is SEQ ID NO: 241 and the second sequence is SEQ ID NO: 242; the first sequence is SEQ ID NO: 249 and the second sequence is SEQ ID NO: 250; the first sequence is SEQ ID NO: 629 and the second sequence is SEQ ID NO: 630; the first sequence is SEQ ID NO: 637 and the second sequence is SEQ ID NO: 638; the first sequence is SEQ ID NO: 645 and the second sequence is SEQ ID NO: 646; the first sequence is SEQ ID NO: 653 and the second sequence is SEQ ID NO: 654; the first sequence is SEQ ID NO: 661 and the second sequence is SEQ ID NO: 662; the first sequence is SEQ ID NO: 669 and the second sequence is SEQ ID NO: 670; the first sequence is SEQ ID NO: 677 and the second sequence is SEQ ID NO: 678; the first sequence is SEQ ID NO: 685 and the second sequence is SEQ ID NO: 686; the first sequence is SEQ ID NO: 693 and the second sequence is SEQ ID NO: 694; the first sequence is SEQ ID NO: 701 and the second sequence is SEQ ID NO: 702; the first sequence is SEQ ID NO: 703 and the second sequence is SEQ ID NO: 704; the first sequence is SEQ ID NO: 711 and the second sequence is SEQ ID NO: 712; the first sequence is SEQ ID NO: 713 and the second sequence is SEQ ID NO: 714; the first sequence is SEQ ID NO: 715 and the second sequence is SEQ ID NO: 716; or the first sequence is SEQ ID NO: 1027 and the second sequence is SEQ ID NO: 1028.

In some embodiments, the L moiety of compound 3.1 comprises an antibody comprising a heavy chain variable region having at least 80% sequence identity to a first sequence and a light chain variable region having at least 80% sequence identity to a second sequence, wherein: the first sequence is SEQ ID NO: 19 and the second sequence is SEQ ID NO: 20; the first sequence is SEQ ID NO: 83 and the second sequence is SEQ ID NO: 84; the first sequence is SEQ ID NO: 97 and the second sequence is SEQ ID NO: 98; the first sequence is SEQ ID NO: 105 and the second sequence is SEQ ID NO: 106; the first sequence is SEQ ID NO: 113 and the second sequence is SEQ ID NO: 114; the first sequence is SEQ ID NO: 121 and the second sequence is SEQ ID NO: 122; the first sequence is SEQ ID NO: 129 and the second sequence is SEQ ID NO: 130; the first sequence is SEQ ID NO: 137 and the second sequence is SEQ ID NO: 138; the first sequence is SEQ ID NO: 153 and the second sequence is SEQ ID NO: 154; the first sequence is SEQ ID NO: 161 and the second sequence is SEQ ID NO: 162; the first sequence is SEQ ID NO: 169 and the second sequence is SEQ ID NO: 170; the first sequence is SEQ ID NO: 177 and the second sequence is SEQ ID NO: 178; the first sequence is SEQ ID NO: 185 and the second sequence is SEQ ID NO: 186; the first sequence is SEQ ID NO: 193 and the second sequence is SEQ ID NO: 194; the first sequence is SEQ ID NO: 201 and the second sequence is SEQ ID NO: 202; the first sequence is SEQ ID NO: 209 and the second sequence is SEQ ID NO: 210; the first sequence is SEQ ID NO: 217 and the second sequence is SEQ ID NO: 218; the first sequence is SEQ ID NO: 225 and the second sequence is SEQ ID NO: 226; the first sequence is SEQ ID NO: 233 and the second sequence is SEQ ID NO: 234; the first sequence is SEQ ID NO: 241 and the second sequence is SEQ ID NO: 242; the first sequence is SEQ ID NO: 249 and the second sequence is SEQ ID NO: 250; or the first sequence is SEQ ID NO: 1027 and the second sequence is SEQ ID NO: 1028. In some cases, the first sequence is SEQ ID NO: 19 and the second sequence is SEQ ID NO: 20.

In some embodiments, the heavy chain variable region of the L moiety of compound 3.1 has at least 85% sequence identity to the first sequence and the light chain variable region has at least 85% sequence identity to the second sequence. In some cases, the heavy chain variable region has at least 90% sequence identity to the first sequence and the light chain variable region has at least 90% sequence identity to the second sequence. In some cases, the heavy chain variable region has at least 95% sequence identity to the first sequence and the light chain variable region has at least 95% sequence identity to the second sequence. In some cases, the heavy chain variable region has at least 98% sequence identity to the first sequence and the light chain variable region has at least 98% sequence identity to the second sequence. In some cases, the heavy chain variable region has at least 99% sequence identity to the first sequence and the light chain variable region has at least 99% sequence identity to the second sequence. In some cases, the heavy chain variable region comprises the first sequence and the light chain variable region comprises the second sequence.

(vi) Heavy and Light Chains

In some embodiments, the L moiety of compound 3.1 comprises an antibody comprising a heavy chain having at least 80% sequence identity to a first sequence and a light chain having at least 80% sequence identity to a second sequence, wherein: the first sequence is SEQ ID NO: 1 and the second sequence is SEQ ID NO: 5; the first sequence is SEQ ID NO: 2 and the second sequence is SEQ ID NO: 5; the first sequence is SEQ ID NO: 3 and the second sequence is SEQ ID NO: 5; the first sequence is SEQ ID NO: 4 and the second sequence is SEQ ID NO: 5; the first sequence is SEQ ID NO: 6 and the second sequence is SEQ ID NO: 10; the first sequence is SEQ ID NO: 7 and the second sequence is SEQ ID NO: 10; the first sequence is SEQ ID NO: 8 and the second sequence is SEQ ID NO: 10; the first sequence is SEQ ID NO: 9 and the second sequence is SEQ ID NO: 10; the first sequence is SEQ ID NO: 46 and the second sequence is SEQ ID NO: 48; the first sequence is SEQ ID NO: 47 and the second sequence is SEQ ID NO: 48; the first sequence is SEQ ID NO: 57 and the second sequence is SEQ ID NO: 59; the first sequence is SEQ ID NO: 58 and the second sequence is SEQ ID NO: 59; the first sequence is SEQ ID NO: 60 and the second sequence is SEQ ID NO: 62; the first sequence is SEQ ID NO: 61 and the second sequence is SEQ ID NO: 62; the first sequence is SEQ ID NO: 71 and the second sequence is SEQ ID NO: 73; the first sequence is SEQ ID NO: 72 and the second sequence is SEQ ID NO: 73; the first sequence is SEQ ID NO: 74 and the second sequence is SEQ ID NO: 76; the first sequence is SEQ ID NO: 75 and the second sequence is SEQ ID NO: 76; the first sequence is SEQ ID NO: 85 and the second sequence is SEQ ID NO: 87; the first sequence is SEQ ID NO: 86 and the second sequence is SEQ ID NO: 87; the first sequence is SEQ ID NO: 88 and the second sequence is SEQ ID NO: 90; the first sequence is SEQ ID NO: 89 and the second sequence is SEQ ID NO: 90; the first sequence is SEQ ID NO: 251 and the second sequence is SEQ ID NO: 252; the first sequence is SEQ ID NO: 253 and the second sequence is SEQ ID NO: 254; the first sequence is SEQ ID NO: 255 and the second sequence is SEQ ID NO: 256; the first sequence is SEQ ID NO: 257 and the second sequence is SEQ ID NO: 258; the first sequence is SEQ ID NO: 259 and the second sequence is SEQ ID NO: 260; the first sequence is SEQ ID NO: 261 and the second sequence is SEQ ID NO: 262; the first sequence is SEQ ID NO: 263 and the second sequence is SEQ ID NO: 264; the first sequence is SEQ ID NO: 265 and the second sequence is SEQ ID NO: 266; the first sequence is SEQ ID NO: 267 and the second sequence is SEQ ID NO: 268; the first sequence is SEQ ID NO: 269 and the second sequence is SEQ ID NO: 270; the first sequence is SEQ ID NO: 271 and the second sequence is SEQ ID NO: 272; the first sequence is SEQ ID NO: 273 and the second sequence is SEQ ID NO: 274; the first sequence is SEQ ID NO: 275 and the second sequence is SEQ ID NO: 276; the first sequence is SEQ ID NO: 277 and the second sequence is SEQ ID NO: 278; the first sequence is SEQ ID NO: 279 and the second sequence is SEQ ID NO: 280; the first sequence is SEQ ID NO: 281 and the second sequence is SEQ ID NO: 282; the first sequence is SEQ ID NO: 283 and the second sequence is SEQ ID NO: 284; the first sequence is SEQ ID NO: 285 and the second sequence is SEQ ID NO: 286; the first sequence is SEQ ID NO: 287 and the second sequence is SEQ ID NO: 288; the first sequence is SEQ ID NO: 289 and the second sequence is SEQ ID NO: 290; the first sequence is SEQ ID NO: 299 and the second sequence is SEQ ID NO: 300; the first sequence is SEQ ID NO: 493 and the second sequence is SEQ ID NO: 494; the first sequence is SEQ ID NO: 717 and the second sequence is SEQ ID NO: 718; the first sequence is SEQ ID NO: 719 and the second sequence is SEQ ID NO: 720; the first sequence is SEQ ID NO: 721 and the second sequence is SEQ ID NO: 722; the first sequence is SEQ ID NO: 723 and the second sequence is SEQ ID NO: 724; or the first sequence is SEQ ID NO: 757 and the second sequence is SEQ ID NO: 758.

In some embodiments, the L moiety of compound 3.1 comprises an antibody comprising a heavy chain having at least 80% sequence identity to a first sequence and a light chain having at least 80% sequence identity to a second sequence, wherein: the first sequence is SEQ ID NO: 1 and the second sequence is SEQ ID NO: 5; the first sequence is SEQ ID NO: 2 and the second sequence is SEQ ID NO: 5; the first sequence is SEQ ID NO: 3 and the second sequence is SEQ ID NO: 5; the first sequence is SEQ ID NO: 4 and the second sequence is SEQ ID NO: 5; the first sequence is SEQ ID NO: 6 and the second sequence is SEQ ID NO: 10; the first sequence is SEQ ID NO: 7 and the second sequence is SEQ ID NO: 10; the first sequence is SEQ ID NO: 8 and the second sequence is SEQ ID NO: 10; the first sequence is SEQ ID NO: 9 and the second sequence is SEQ ID NO: 10; the first sequence is SEQ ID NO: 85 and the second sequence is SEQ ID NO: 87; the first sequence is SEQ ID NO: 86 and the second sequence is SEQ ID NO: 87; the first sequence is SEQ ID NO: 88 and the second sequence is SEQ ID NO: 90; the first sequence is SEQ ID NO: 89 and the second sequence is SEQ ID NO: 90; the first sequence is SEQ ID NO: 251 and the second sequence is SEQ ID NO: 252; the first sequence is SEQ ID NO: 253 and the second sequence is SEQ ID NO: 254; the first sequence is SEQ ID NO: 255 and the second sequence is SEQ ID NO: 256; the first sequence is SEQ ID NO: 257 and the second sequence is SEQ ID NO: 258; the first sequence is SEQ ID NO: 259 and the second sequence is SEQ ID NO: 260; the first sequence is SEQ ID NO: 261 and the second sequence is SEQ ID NO: 262; the first sequence is SEQ ID NO: 263 and the second sequence is SEQ ID NO: 264; the first sequence is SEQ ID NO: 265 and the second sequence is SEQ ID NO: 266; the first sequence is SEQ ID NO: 267 and the second sequence is SEQ ID NO: 268; the first sequence is SEQ ID NO: 269 and the second sequence is SEQ ID NO: 270; the first sequence is SEQ ID NO: 271 and the second sequence is SEQ ID NO: 272; the first sequence is SEQ ID NO: 273 and the second sequence is SEQ ID NO: 274; the first sequence is SEQ ID NO: 275 and the second sequence is SEQ ID NO: 276; the first sequence is SEQ ID NO: 277 and the second sequence is SEQ ID NO: 278; the first sequence is SEQ ID NO: 279 and the second sequence is SEQ ID NO: 280; the first sequence is SEQ ID NO: 281 and the second sequence is SEQ ID NO: 282; the first sequence is SEQ ID NO: 283 and the second sequence is SEQ ID NO: 284; the first sequence is SEQ ID NO: 285 and the second sequence is SEQ ID NO: 286; the first sequence is SEQ ID NO: 287 and the second sequence is SEQ ID NO: 288; the first sequence is SEQ ID NO: 289 and the second sequence is SEQ ID NO: 290; the first sequence is SEQ ID NO: 717 and the second sequence is SEQ ID NO: 718; the first sequence is SEQ ID NO: 719 and the second sequence is SEQ ID NO: 720; the first sequence is SEQ ID NO: 721 and the second sequence is SEQ ID NO: 722; or the first sequence is SEQ ID NO: 723 and the second sequence is SEQ ID NO: 724.

In some embodiments, the L moiety of compound 3.1 comprises an antibody comprising a heavy chain having at least 80% sequence identity to a first sequence and a light chain having at least 80% sequence identity to a second sequence, wherein: the first sequence is SEQ ID NO: 1 and the second sequence is SEQ ID NO: 5; the first sequence is SEQ ID NO: 2 and the second sequence is SEQ ID NO: 5; the first sequence is SEQ ID NO: 3 and the second sequence is SEQ ID NO: 5; the first sequence is SEQ ID NO: 4 and the second sequence is SEQ ID NO: 5; the first sequence is SEQ ID NO: 6 and the second sequence is SEQ ID NO: 10; the first sequence is SEQ ID NO: 7 and the second sequence is SEQ ID NO: 10; the first sequence is SEQ ID NO: 8 and the second sequence is SEQ ID NO: 10; the first sequence is SEQ ID NO: 9 and the second sequence is SEQ ID NO: 10; the first sequence is SEQ ID NO: 85 and the second sequence is SEQ ID NO: 87; the first sequence is SEQ ID NO: 86 and the second sequence is SEQ ID NO: 87; the first sequence is SEQ ID NO: 88 and the second sequence is SEQ ID NO: 90; the first sequence is SEQ ID NO: 89 and the second sequence is SEQ ID NO: 90; the first sequence is SEQ ID NO: 251 and the second sequence is SEQ ID NO: 252; the first sequence is SEQ ID NO: 253 and the second sequence is SEQ ID NO: 254; the first sequence is SEQ ID NO: 255 and the second sequence is SEQ ID NO: 256; the first sequence is SEQ ID NO: 257 and the second sequence is SEQ ID NO: 258; the first sequence is SEQ ID NO: 259 and the second sequence is SEQ ID NO: 260; the first sequence is SEQ ID NO: 261 and the second sequence is SEQ ID NO: 262; the first sequence is SEQ ID NO: 263 and the second sequence is SEQ ID NO: 264; the first sequence is SEQ ID NO: 265 and the second sequence is SEQ ID NO: 266; the first sequence is SEQ ID NO: 267 and the second sequence is SEQ ID NO: 268; the first sequence is SEQ ID NO: 269 and the second sequence is SEQ ID NO: 270; the first sequence is SEQ ID NO: 271 and the second sequence is SEQ ID NO: 272; the first sequence is SEQ ID NO: 273 and the second sequence is SEQ ID NO: 274; the first sequence is SEQ ID NO: 275 and the second sequence is SEQ ID NO: 276; the first sequence is SEQ ID NO: 277 and the second sequence is SEQ ID NO: 278; the first sequence is SEQ ID NO: 279 and the second sequence is SEQ ID NO: 280; the first sequence is SEQ ID NO: 281 and the second sequence is SEQ ID NO: 282; the first sequence is SEQ ID NO: 283 and the second sequence is SEQ ID NO: 284; the first sequence is SEQ ID NO: 285 and the second sequence is SEQ ID NO: 286; the first sequence is SEQ ID NO: 287 and the second sequence is SEQ ID NO: 288; or the first sequence is SEQ ID NO: 289 and the second sequence is SEQ ID NO: 290.

In some embodiments, the L moiety of compound 3.1 comprises an antibody comprising a heavy chain having at least 80% sequence identity to a first sequence and a light chain having at least 80% sequence identity to a second sequence, wherein: the first sequence is SEQ ID NO: 1 and the second sequence is SEQ ID NO: 5; the first sequence is SEQ ID NO: 2 and the second sequence is SEQ ID NO: 5; the first sequence is SEQ ID NO: 3 and the second sequence is SEQ ID NO: 5; the first sequence is SEQ ID NO: 4 and the second sequence is SEQ ID NO: 5; the first sequence is SEQ ID NO: 6 and the second sequence is SEQ ID NO: 10; the first sequence is SEQ ID NO: 7 and the second sequence is SEQ ID NO: 10; the first sequence is SEQ ID NO: 8 and the second sequence is SEQ ID NO: 10; or the first sequence is SEQ ID NO: 9 and the second sequence is SEQ ID NO: 10.

In some embodiments, the L moiety of is an antibody comprising a heavy chain having at least 80% sequence identity to a first sequence and a light chain having at least 80% sequence identity to a second sequence, wherein: the first sequence is SEQ ID NO: 3 or SEQ ID NO: 4 and the second sequence is SEQ ID NO: 5.

In some embodiments, the L moiety of compound 3.1 comprises a heavy chain with at least 85% sequence identity to the first sequence and the light chain has at least 85% sequence identity to the second sequence. In some cases, the heavy chain has at least 90% sequence identity to the first sequence and the light chain has at least 90% sequence identity to the second sequence. In some cases, the heavy chain has at least 95% sequence identity to the first sequence and the light chain has at least 95% sequence identity to the second sequence. In some cases, the heavy chain has at least 98% sequence identity to the first sequence and the light chain has at least 98% sequence identity to the second sequence. In some cases, the heavy chain has at least 99% sequence identity to the first sequence and the light chain has at least 99% sequence identity to the second sequence. In some cases, the heavy chain comprises the first sequence and the light chain comprises the second sequence.

(vii) Complementarity-Determining Regions

In some embodiments, the L moiety of compound 3.1 comprises an antibody comprising CDR-H1, CDR-H2, CDR-H3, CDR-L1, CDR-L2, and CDR-L3 comprising at least 80% sequence identity to: SEQ ID NO: 13, 14, 15, 16, 17, and 18, respectively (i.e., CDR-H1 has at least 80% sequence identity to SEQ ID NO: 13, CDR-H2 has at least 80% sequence identity to SEQ ID NO: 14, CDR-H3 has at least 80% sequence identity to SEQ ID NO: 15, CDR-L1 has at least 80% sequence identity to SEQ ID NO: 16, CDR-L2 has at least 80% sequence identity to SEQ ID NO: 17, and CDR-L3 has at least 80% sequence identity to SEQ ID NO: 18); SEQ ID NO: 21, 22, 23, 24, 25, and 26, respectively; SEQ ID NO: 38, 39, 40, 41, 42, and 43, respectively; SEQ ID NO: 49, 50, 51, 52, 53, and 54, respectively; SEQ ID NO: 63, 64, 65, 66, 67, and 68, respectively; SEQ ID NO: 77, 78, 79, 80, 81, and 82, respectively; SEQ ID NO: 91, 92, 93, 94, 95, and 96, respectively; SEQ ID NO: 99, 100, 101, 102, 103, and 104, respectively; SEQ ID NO: 107, 108, 109, 110, 111, and 112, respectively; SEQ ID NO: 115, 116, 117, 118, 119, and 120, respectively; SEQ ID NO: 123, 124, 125, 126, 127, and 128, respectively; SEQ ID NO: 131, 132, 133, 134, 135, and 136, respectively; SEQ ID NO: 139, 140, 141, 142, 143, and 144, respectively; SEQ ID NO: 147, 148, 149, 150, 151, and 152, respectively; SEQ ID NO: 155, 156, 157, 158, 159, and 160, respectively; SEQ ID NO: 163, 164, 165, 166, 167, and 168, respectively; SEQ ID NO: 171, 172, 173, 174, 175, and 176, respectively; SEQ ID NO: 179, 180, 181, 182, 183, and 184, respectively; SEQ ID NO: 187, 188, 189, 190, 191, and 192, respectively; SEQ ID NO: 195, 196, 197, 198, 199, and 200, respectively; SEQ ID NO: 203, 204, 205, 206, 207 and 208, respectively; SEQ ID NO: 211, 212, 213, 214, 215, and 216, respectively; SEQ ID NO: 219, 220, 221, 222, 223, and 224, respectively; SEQ ID NO: 227, 228, 229, 230, 231, and 232, respectively; SEQ ID NO: 235, 236, 237, 238, 239, and 240, respectively; SEQ ID NO: 243, 244, 245, 246, 247, and 248, respectively; SEQ ID NO: 291, 292, 293, 294, 295, and 296, respectively; SEQ ID NO: 301, 302, 303, 304, 305, and 306, respectively; SEQ ID NO: 309, 310, 311, 312, 313, and 314, respectively; SEQ ID NO: 317, 318, 319, 320, 321, and 322, respectively; SEQ ID NO: 325, 326, 327, 328, 329, and 330, respectively; SEQ ID NO: 333, 334, 335, 336, 337, and 338, respectively; SEQ ID NO: 341, 342, 343, 344, 345, and 346, respectively; SEQ ID NO: 349, 350, 351, 352, 353, and 354, respectively; SEQ ID NO: 357, 358, 359, 360, 361, and 362, respectively; SEQ ID NO: 365, 366, 367, 368, 369, and 370, respectively; SEQ ID NO: 373, 374, 375, 376, 377, and 378, respectively; SEQ ID NO: 381, 382, 383, 384, 385, and 386, respectively; SEQ ID NO: 389, 390, 391, 392, 393, and 394, respectively; SEQ ID NO: 397, 398, 399, 400, 401, and 402, respectively; SEQ ID NO: 405, 406, 407, 408, 409, and 410, respectively; SEQ ID NO: 413, 414, 415, 416, 417, and 418, respectively; SEQ ID NO: 421, 422, 423, 424, 425, and 426, respectively; SEQ ID NO: 429, 430, 431, 432, 433, and 434, respectively; SEQ ID NO: 437, 438, 439, 440, 441, and 442, respectively; SEQ ID NO: 445, 446, 447, 448, 449, and 450, respectively; SEQ ID NO: 453, 454, 455, 456, 457, and 458, respectively; SEQ ID NO: 461, 462, 463, 464, 465, and 466, respectively; SEQ ID NO: 469, 470, 471, 472, 473, and 474, respectively; SEQ ID NO: 477, 478, 479, 480, 481, and 482, respectively; SEQ ID NO: 485, 486, 487, 488, 489, and 490, respectively; SEQ ID NO: 495, 496, 497, 498, 499, and 500, respectively; SEQ ID NO: 503, 504, 505, 506, 507, and 508, respectively; SEQ ID NO: 511, 512, 513, 514, 515, and 516, respectively; SEQ ID NO: 519, 520, 521, 522, 523, and 524, respectively; SEQ ID NO: 527, 528, 529, 530, 531, and 532, respectively; SEQ ID NO: 535, 536, 537, 538, 539, and 540, respectively; SEQ ID NO: 543, 544, 545, 546, 547, and 548, respectively; SEQ ID NO: 551, 552, 553, 554, 555, and 556, respectively; SEQ ID NO: 559, 560, 561, 562, 563, and 564, respectively; SEQ ID NO: 567, 568, 569, 570, 571, and 572, respectively; SEQ ID NO: 575, 576, 577, 578, 579, and 580, respectively; SEQ ID NO: 583, 584, 585, 586, 587, and 588, respectively; SEQ ID NO: 591, 592, 593, 594, 595, and 596, respectively; SEQ ID NO: 599, 600, 601, 602, 603, and 604, respectively; SEQ ID NO: 607, 608, 609, 610, 611, and 612, respectively; SEQ ID NO: 615, 616, 617, 618, 619, and 620, respectively; SEQ ID NO: 623, 624, 625, 626, 627, and 628, respectively; SEQ ID NO: 631, 632, 633, 634, 635, and 636, respectively; SEQ ID NO: 639, 640, 641, 642, 643, and 644, respectively; SEQ ID NO: 647, 648, 649, 650, 651, and 652, respectively; SEQ ID NO: 655, 656, 657, 658, 659, and 660, respectively; SEQ ID NO: 663, 664, 665, 666, 667, and 668, respectively; SEQ ID NO: 671, 672, 673, 674, 675, and 676, respectively; SEQ ID NO: 679, 680, 681, 682, 683, and 684, respectively; SEQ ID NO: 687, 688, 689, 690, 691, and 692, respectively; SEQ ID NO: 695, 696, 697, 698, 699, and 700, respectively; SEQ ID NO: 705, 706, 707, 708, 709, and 710, respectively; SEQ ID NO: 725, 726, 727, 728, 729, and 730, respectively; SEQ ID NO: 733, 734, 735, 736, 737, and 738, respectively; SEQ ID NO: 741, 742, 743, 744, 745, and 746, respectively; SEQ ID NO: 749, 750, 751, 752, 753, and 754, respectively; SEQ ID NO: 759, 760, 761, 762, 763, and 764, respectively; SEQ ID NO: 767, 768, 769, 770, 771, and 772, respectively; SEQ ID NO: 775, 776, 777, 778, 779, and 780, respectively; SEQ ID NO: 783, 784, 785, 786, 787, and 788, respectively; SEQ ID NO: 791, 792, 793, 794, 795, and 796, respectively; SEQ ID NO: 799, 800, 801, 802, 803, and 804, respectively; SEQ ID NO: 807, 808, 809, 810, 811, and 812, respectively; SEQ ID NO: 815, 816, 817, 818, 819, and 820, respectively; SEQ ID NO: 823, 824, 825, 826, 827, and 828, respectively; SEQ ID NO: 831, 832, 833, 834, 835, and 836, respectively; SEQ ID NO: 839, 840, 841, 842, 843, and 844, respectively; SEQ ID NO: 847, 848, 849, 850, 851, and 852, respectively; SEQ ID NO: 855, 856, 857, 858, 859, and 860, respectively; SEQ ID NO: 863, 864, 865, 866, 867, and 868, respectively; SEQ ID NO: 871, 872, 873, 874, 875, and 876, respectively; SEQ ID NO: 879, 880, 881, 882, 883, and 884, respectively; SEQ ID NO: 887, 888, 889, 890, 891, and 892, respectively; SEQ ID NO: 895, 896, 897, 898, 899, and 900, respectively; SEQ ID NO: 903, 904, 905, 906, 907, and 908, respectively; SEQ ID NO: 911, 912, 913, 914, 915, and 916, respectively; SEQ ID NO: 919, 920, 921, 922, 923, and 924, respectively; SEQ ID NO: 927, 928, 929, 930, 931, and 932, respectively; SEQ ID NO: 935, 936, 937, 938, 939, and 940, respectively; SEQ ID NO: 945, 946, 947, 948, 949, and 950, respectively; SEQ ID NO: 953, 954, 955, 956, 957, and 958, respectively; SEQ ID NO: 961, 962, 963, 964, 965, and 966, respectively; SEQ ID NO: 969, 970, 971, 972, 973, and 974, respectively; SEQ ID NO: 977, 978, 979, 980, 981, and 982, respectively; SEQ ID NO: 985, 986, 987, 988, 989, and 990, respectively; SEQ ID NO: 997, 998, 999, 1000, 1001, and 1002, respectively; SEQ ID NO: 1005, 1006, 1007, 1008, 1009, and 1010, respectively; SEQ ID NO: 1013, 1014, 1015, 1016, 1017, and 1018, respectively; SEQ ID NO: 1021, 1022, 1023, 1024, 1025, and 1026, respectively; SEQ ID NO: 1029, 1030, 1031, 1032, 1033, and 1034, respectively; or SEQ ID NO: 1037, 1038, 1039, 1040, 1041, and 1042, respectively.

In some cases, the L moiety of compound 3.1 comprises an antibody that targets an immune checkpoint selected from the group consisting of PDL1, B7H4, B7H3, and TIGIT. For example, in some cases, the antibody comprises CDR-H1, CDR-H2, CDR-H3, CDR-L1, CDR-L2, and CDR-L3 comprising at least 80% sequence identity to: SEQ ID NO: 13, 14, 15, 16, 17, and 18, respectively; SEQ ID NO: 77, 78, 79, 80, 81, and 82, respectively; SEQ ID NO: 91, 92, 93, 94, 95, and 96, respectively; SEQ ID NO: 99, 100, 101, 102, 103, and 104, respectively; SEQ ID NO: 107, 108, 109, 110, 111, and 112, respectively; SEQ ID NO: 115, 116, 117, 118, 119, and 120, respectively; SEQ ID NO: 123, 124, 125, 126, 127, and 128, respectively; SEQ ID NO: 131, 132, 133, 134, 135, and 136, respectively; SEQ ID NO: 139, 140, 141, 142, 143, and 144, respectively; SEQ ID NO: 147, 148, 149, 150, 151, and 152, respectively; SEQ ID NO: 155, 156, 157, 158, 159, and 160, respectively; SEQ ID NO: 163, 164, 165, 166, 167, and 168, respectively; SEQ ID NO: 171, 172, 173, 174, 175, and 176, respectively; SEQ ID NO: 179, 180, 181, 182, 183, and 184, respectively; SEQ ID NO: 187, 188, 189, 190, 191, and 192, respectively; SEQ ID NO: 195, 196, 197, 198, 199, and 200, respectively; SEQ ID NO: 203, 204, 205, 206, 207 and 208, respectively; SEQ ID NO: 211, 212, 213, 214, 215, and 216, respectively; SEQ ID NO: 219, 220, 221, 222, 223, and 224, respectively; SEQ ID NO: 227, 228, 229, 230, 231, and 232, respectively; SEQ ID NO: 235, 236, 237, 238, 239, and 240, respectively; SEQ ID NO: 243, 244, 245, 246, 247, and 248, respectively; SEQ ID NO: 623, 624, 625, 626, 627, and 628, respectively; SEQ ID NO: 631, 632, 633, 634, 635, and 636, respectively; SEQ ID NO: 639, 640, 641, 642, 643, and 644, respectively; SEQ ID NO: 647, 648, 649, 650, 651, and 652, respectively; SEQ ID NO: 655, 656, 657, 658, 659, and 660, respectively; SEQ ID NO: 663, 664, 665, 666, 667, and 668, respectively; SEQ ID NO: 671, 672, 673, 674, 675, and 676, respectively; SEQ ID NO: 679, 680, 681, 682, 683, and 684, respectively; SEQ ID NO: 687, 688, 689, 690, 691, and 692, respectively; SEQ ID NO: 695, 696, 697, 698, 699, and 700, respectively; SEQ ID NO: 705, 706, 707, 708, 709, and 710, respectively; or SEQ ID NO: 1021, 1022, 1023, 1024, 1025, and 1026, respectively.

In some embodiments, the CDR-H1, CDR-H2, CDR-H3, CDR-L1, CDR-L2, and CDR-L3 comprise at least 85% sequence identity to the respective 6 indicated sequences. In some cases, the CDR-H1, CDR-H2, CDR-H3, CDR-L1, CDR-L2, and CDR-L3 each comprise at least 90% sequence identity to the respective 6 indicated sequences. In some cases, the CDR-H1, CDR-H2, CDR-H3, CDR-L1, CDR-L2, and CDR-L3 each comprise at least 95% sequence identity to the respective 6 indicated sequences. In some cases, the CDR-H1, CDR-H2, CDR-H3, CDR-L1, CDR-L2, and CDR-L3 each comprise at most one mutation relative to the respective 6 indicated sequences.

(viii) Exemplary Antibodies

In some cases, the L moiety of compound 3.1 comprises an antibody that binds to PDL1. PDL1 is expressed in a broad range of cancers, as well as certain immune cells, and often serves as a primary mechanism for immune suppression in tumor microenvironments. The PDL1 agonism of PD-1 can act as an immune checkpoint, diminishing lymphocyte tumor infiltration, and T-cell receptor mediated proliferation and signaling. While PDL1 antagonism can reverse immune suppression, PDL1 targeting can also localize treatments to the sites of cancers, allowing drugs to selectively target cancer cells or stimulate immune responses in the presence of tumors.

In some embodiments, the compound 3.1 comprises an anti-PDL1 antibody comprising CDR-H1, CDR-H2, CDR-H3, CDR-L1, CDR-L2, and CDR-L3 comprising at least 80% sequence identity, at least 85% sequence identity, at least 90% sequence identity, at least 95%, at least 98%, or at least 99% sequence identity to the amino acid sequences of SEQ ID NOs: 13, 14, 15, 16, 17, and 18 respectively. In some cases, the antibody comprises CDR-H1, CDR-H2, CDR-H3, CDR-L1, CDR-L2, and CDR-L3 each comprising at most one mutation relative to the amino acid sequences of SEQ ID NOs: 13, 14, 15, 16, 17, and 18, respectively. In some embodiments, a Ligand-Drug Conjugate corresponding to compound 3.1 in Table 3 is provided wherein L comprises any of the anti-PDL1 antibodies described above or L comprises an amino acid sequence with at least 80% sequence identity, at least 85% sequence identity, at least 90% sequence identity, at least 95%, at least 98%, or at least 99% sequence identity an amino acid sequences of SEQ ID NOs: 13, 14, 15, 16, 17, or 18.

In some embodiments, the L moiety of compound 3.1 comprises an antibody comprising a heavy chain comprising an amino acid sequence that is at least 80%, at least 85%, at least 90%, at least 95%, at least 98%, or 99% identical to the amino acid sequence of any one of SEQ ID NO: 1-4 and a light chain comprising an amino acid sequence that is at least 95% at least 96%, at least 97%, at least 98%, at least 99%, or 100% identical to the amino acid sequence of SEQ ID NO: 5. In some embodiments, the antibody comprises a heavy chain comprising an amino acid sequence that is at least 80%, at least 85%, at least 90%, at least 95%, at least 98%, or 99% identical to the amino acid sequence of SEQ ID NO: 1 and a light chain comprising an amino acid sequence that is at least 95% at least 96%, at least 97%, at least 98%, at least 99%, or 100% identical to the amino acid sequence of SEQ ID NO: 5. In some embodiments, the antibody comprises a heavy chain comprising an amino acid sequence that is at least 80%, at least 85%, at least 90%, at least 95%, at least 98%, or 99% identical to the amino acid sequence of SEQ ID NO: 2 and a light chain comprising an amino acid sequence that is at least 95% at least 96%, at least 97%, at least 98%, at least 99%, or 100% identical to the amino acid sequence of SEQ ID NO: 5. In some embodiments, the antibody comprises a heavy chain comprising an amino acid sequence that is at least 80%, at least 85%, at least 90%, at least 95%, at least 98%, or 99% identical to the amino acid sequence of SEQ ID NO: 3 and a light chain comprising an amino acid sequence that is at least 95% at least 96%, at least 97%, at least 98%, at least 99%, or 100% identical to the amino acid sequence of SEQ ID NO: 5. In some embodiments, the antibody comprises a heavy chain comprising an amino acid sequence that is at least 80%, at least 85%, at least 90%, at least 95%, at least 98%, or 99% identical to the amino acid sequence of SEQ ID NO: 4 and a light chain comprising an amino acid sequence that is at least 95% at least 96%, at least 97%, at least 98%, at least 99%, or 100% identical to the amino acid sequence of SEQ ID NO: 5.

In some embodiments, the L moiety of compound 3.1 comprises an antibody comprising a heavy chain variable region comprising an amino acid sequence that is at least 80%, at least 85%, at least 90%, at least 95%, at least 98%, or 99% identical to the amino acid sequence of any one of SEQ ID NO: 6-9 and a light chain variable region comprising an amino acid sequence that is at least 95% at least 96%, at least 97%, at least 98%, at least 99%, or 100% identical to the amino acid sequence of SEQ ID NO: 10. In some embodiments, the antibody comprises a heavy chain variable region comprising an amino acid sequence that is at least 80%, at least 85%, at least 90%, at least 95%, at least 98%, or 99% identical to the amino acid sequence of any one of SEQ ID NO: 6 and a light chain variable region comprising an amino acid sequence that is at least 95% at least 96%, at least 97%, at least 98%, at least 99%, or 100% identical to the amino acid sequence of SEQ ID NO: 10. In some embodiments, the antibody comprises a heavy chain variable region comprising an amino acid sequence that is at least 80%, at least 85%, at least 90%, at least 95%, at least 98%, or 99% identical to the amino acid sequence of any one of SEQ ID NO: 7 and a light chain variable region comprising an amino acid sequence that is at least 95% at least 96%, at least 97%, at least 98%, at least 99%, or 100% identical to the amino acid sequence of SEQ ID NO: 10. In some embodiments, the antibody comprises a heavy chain variable region comprising an amino acid sequence that is at least 80%, at least 85%, at least 90%, at least 95%, at least 98%, or 99% identical to the amino acid sequence of any one of SEQ ID NO: 8 and a light chain variable region comprising an amino acid sequence that is at least 95% at least 96%, at least 97%, at least 98%, at least 99%, or 100% identical to the amino acid sequence of SEQ ID NO: 10. In some embodiments, the antibody comprises a heavy chain variable region comprising an amino acid sequence that is at least 80%, at least 85%, at least 90%, at least 95%, at least 98%, or 99% identical to the amino acid sequence of any one of SEQ ID NO: 9 and a light chain variable region comprising an amino acid sequence that is at least 95% at least 96%, at least 97%, at least 98%, at least 99%, or 100% identical to the amino acid sequence of SEQ ID NO: 10.

In some embodiments, the L moiety of compound 3.1 comprises an antibody comprising a heavy chain variable region comprising an amino acid sequence that is at least 80%, at least 85%, at least 90%, at least 95%, at least 98%, or 99% identical to the amino acid sequence of SEQ ID NO: 19 and a light chain variable region comprising an amino acid sequence that is at least 95% at least 96%, at least 97%, at least 98%, at least 99%, or 100% identical to the amino acid sequence of SEQ ID NO: 20.

In some embodiments, the L moiety of compound 3.1 comprises an antibody that binds to EphA2. In some embodiments, the antibody comprises CDR-H1, CDR-H2, CDR-H3, CDR-L1, CDR-L2, and CDR-L3 comprising the amino acid sequences that are at least 80%, at least 85%, at least 90%, at least 95%, at least 98%, or at least 99% identical to the amino acid sequences of SEQ ID NOs: 21, 22, 23, 24, 25, and 26, respectively.

In some embodiments, the anti-EphA2 antibody comprises a heavy chain variable region comprising an amino acid sequence that is at least 80%, at least 85%, at least 90%, at least 95%, at least 98%, or at least 99% identical to the amino acid sequence of SEQ ID NO: 27 and a light chain variable region comprising an amino acid sequence that is at least 80%, at least 85%, at least 90%, at least 95%, at least 98%, or at least 99% identical to the amino acid sequence of SEQ ID NO: 28. In some embodiments, the anti-EphA2 antibody comprises a heavy chain comprising an amino acid sequence that is at least 80%, at least 85%, at least 90%, at least 95%, at least 98%, or at least 99% identical to the amino acid sequence of SEQ ID NO: 29 or SEQ ID NO: 30 and a light chain comprising an amino acid sequence that is at least 80%, at least 85%, at least 90%, at least 95%, at least 98%, or at least 99% identical to the amino acid sequence of the amino acid sequence of SEQ ID NO: 31. In some embodiments, the anti-EphA2 antibody comprises a heavy chain comprising the amino acid sequence that is at least 80%, at least 85%, at least 90%, at least 95%, at least 98%, or at least 99% identical to the amino acid sequence of SEQ ID NO: 32 or SEQ ID NO: 33 and a light chain comprising the amino acid sequence of SEQ ID NO: 34. In some embodiments, the anti-EphA2 antibody comprises a heavy chain that is at least 80%, at least 85%, at least 90%, at least 95%, at least 98%, or at least 99% identical to the amino acid sequence of SEQ ID NO: 35 or SEQ ID NO: 36 and a light chain comprising the amino acid sequence of SEQ ID NO: 37. In some embodiments, the antibody is h1C1 or 1C1.

In some embodiments, the anti-EphA2 antibody comprises a heavy chain variable region comprising an amino acid sequence that is at least 80%, at least 85%, at least 90%, at least 95%, at least 98%, or 99% identical to the amino acid sequence of SEQ ID NO: 27 and a light chain variable region comprising an amino acid sequence that is at least 80% at least 85%, at least 90%, at least 95%, at least 98%, or 99% identical to the amino acid sequence of SEQ ID NO: 28.

In some embodiments, the L moiety of compound 3.1 comprises an antibody that binds to CD30. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 38, 39, 40, 41, 42, and 43, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 38, 39, 40, 41, 42, and 43, respectively. In some embodiments, the anti-CD30 antibody comprises a heavy chain comprising an amino acid sequence that is at least 80%, at least 85%, at least 90%, at least 95%, at least 98%, or 99% identical to the amino acid sequence of either SEQ ID NO: 46 or 47 and a light chain comprising an amino acid sequence that is at least 80% at least 85%, at least 90%, at least 95%, at least 98%, or 99% identical to the amino acid sequence of SEQ ID NO: 48. In some embodiments, the anti-CD30 antibody comprises a heavy chain variable region comprising an amino acid sequence that is at least 80%, at least 85%, at least 90%, at least 95%, at least 98%, or 99% identical to the amino acid sequence of SEQ ID NO: 44 and a light chain variable region comprising an amino acid sequence that is at least 80% at least 85%, at least 90%, at least 95%, at least 98%, or 99% identical to the amino acid sequence of SEQ ID NO: 45.

In some embodiments, the L moiety of compound 3.1 comprises an antibody that binds to CD228. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 49, 50, 51, 52, 53, and 54, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 49, 50, 51, 52, 53, and 54, respectively. In some embodiments, the anti-CD228 antibody comprises a heavy chain variable region comprising an amino acid sequence that is at least 80%, at least 85%, at least 90%, at least 95%, at least 98%, or 99% identical to the amino acid sequence of SEQ ID NO: 55 and a light chain variable region comprising an amino acid sequence that is at least 80% at least 85%, at least 90%, at least 95%, at least 98%, or 99% identical to the amino acid sequence of SEQ ID NO: 56. In some embodiments, the anti-CD228 antibody comprises a heavy chain comprising an amino acid sequence that is at least 80%, at least 85%, at least 90%, at least 95%, at least 98%, or 99% identical to the amino acid sequence of either of SEQ ID NO: 57 or 58 and a light chain comprising an amino acid sequence that is at least 80% at least 85%, at least 90%, at least 95%, at least 98%, or 99% identical to the amino acid sequence of SEQ ID NO: 59. In some embodiments, the anti-CD228 antibody comprises a heavy chain comprising an amino acid sequence that is at least 80%, at least 85%, at least 90%, at least 95%, at least 98%, or 99% identical to the amino acid sequence of either of SEQ ID NO: 60 or 61 and a light chain comprising an amino acid sequence that is at least 80% at least 85%, at least 90%, at least 95%, at least 98%, or 99% identical to the amino acid sequence of SEQ ID NO: 62.

In some embodiments, the L moiety of compound 3.1 comprises an antibody that binds to avB6. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 63, 64, 65, 66, 67, and 68, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 63, 64, 65, 66, 67, and 68, respectively. In some embodiments, the anti-H2A2 antibody comprises a heavy chain variable region comprising an amino acid sequence that is at least 80%, at least 85%, at least 90%, at least 95%, at least 98%, or 99% identical to the amino acid sequence of SEQ ID NO: 69 and a light chain variable region comprising an amino acid sequence that is at least 80% at least 85%, at least 90%, at least 95%, at least 98%, or 99% identical to the amino acid sequence of SEQ ID NO: 70. In some embodiments, the anti-H2A2 antibody comprises a heavy chain comprising an amino acid sequence that is at least 80%, at least 85%, at least 90%, at least 95%, at least 98%, or 99% identical to the amino acid sequence of either SEQ ID NO: 71 or 72 and a light chain comprising an amino acid sequence that is at least 80% at least 85%, at least 90%, at least 95%, at least 98%, or 99% identical to the amino acid sequence of SEQ ID NO: 73. In some embodiments, the anti-H2A2 antibody comprises a heavy chain comprising an amino acid sequence that is at least 80%, at least 85%, at least 90%, at least 95%, at least 98%, or 99% identical to the amino acid sequence of either SEQ ID NO: 74 or 75 and a light chain comprising an amino acid sequence that is at least 80% at least 85%, at least 90%, at least 95%, at least 98%, or 99% identical to the amino acid sequence of SEQ ID NO: 76. In some embodiments, the anti-avB6 antibody comprises a heavy chain variable region comprising an amino acid sequence that is at least 80%, at least 85%, at least 90%, at least 95%, at least 98%, or 99% identical to the amino acid sequence of SEQ ID NO: 69 and a light chain variable region comprising an amino acid sequence that is at least 80% at least 85%, at least 90%, at least 95%, at least 98%, or 99% identical to the amino acid sequence of SEQ ID NO: 70. In some embodiments, the anti-avB6 antibody comprises a heavy chain comprising an amino acid sequence that is at least 80%, at least 85%, at least 90%, at least 95%, at least 98%, or 99% identical to the amino acid sequence of either SEQ ID NO: 71 or 72 and a light chain comprising an amino acid sequence that is at least 80% at least 85%, at least 90%, at least 95%, at least 98%, or 99% identical to the amino acid sequence of SEQ ID NO: 73. In some embodiments, the anti-avB6 antibody comprises a heavy chain comprising an amino acid sequence that is at least 80%, at least 85%, at least 90%, at least 95%, at least 98%, or 99% identical to the amino acid sequence of either SEQ ID NO: 74 or 75 and a light chain comprising an amino acid sequence that is at least 80% at least 85%, at least 90%, at least 95%, at least 98%, or 99% identical to the amino acid sequence of SEQ ID NO: 76.

In some embodiments, the L moiety of compound 3.1 comprises an antibody that binds to B7H4. In some cases, the antibody comprises a set of CDR sequences (CDR-H1, CDR-H2, CDR-H3, CDR-L1, CDR-L2, and CDR-L3, respectively) of which each sequence comprises at least 80% sequence identity, at least 85% sequence identity, at least 90% sequence identity, at least 95%, or 100% sequence identity to amino acid sequences from a set of amino acid sequences selected from the group consisting of SEQ ID NOs: 77-82, SEQ ID NOs: 91-96, SEQ ID NOs: 99-104, SEQ ID NOs: 107-112, SEQ ID NOs: 115-120, SEQ ID NOs: 123-128, SEQ ID NOs: 131-136, SEQ ID NOs: 139-144, SEQ ID NOs: 147-152, SEQ ID NOs: 155-160, SEQ ID NOs: 163-168, SEQ ID NOs: 171-176, SEQ ID NOs: 179-184, SEQ ID NOs: 187-192, SEQ ID NOs: 195-200, SEQ ID NOs: 203-208, SEQ ID NOs: 211-216, SEQ ID NOs: 219-224, SEQ ID NOs: 227-232, SEQ ID NOs: 235-240, and SEQ ID NOs: 243-248. In some cases, the antibody comprises a set of CDR sequences (CDR-H1, CDR-H2, CDR-H3, CDR-L1, CDR-L2, and CDR-L3, respectively) each comprising at most one mutation relative to an amino acid sequence from a set of amino acid sequences selected from the group consisting of SEQ ID NOs: 77-82, SEQ ID NOs: 91-96, SEQ ID NOs: 99-104, SEQ ID NOs: 107-112, SEQ ID NOs: 115-120, SEQ ID NOs: 123-128, SEQ ID NOs: 131-136, SEQ ID NOs: 139-144, SEQ ID NOs: 147-152, SEQ ID NOs: 155-160, SEQ ID NOs: 163-168, SEQ ID NOs: 171-176, SEQ ID NOs: 179-184, SEQ ID NOs: 187-192, SEQ ID NOs: 195-200, SEQ ID NOs: 203-208, SEQ ID NOs: 211-216, SEQ ID NOs: 219-224, SEQ ID NOs: 227-232, SEQ ID NOs: 235-240, and SEQ ID NOs: 243-248. In some cases, the anti-B7H4 antibody comprises a heavy chain and a light chain comprising amino acid sequences that are at least 80%, at least 85%, at least 90%, at least 95%, at least 98%, at least 99%, or at least 100% identical to the amino acid sequences of SEQ ID NO: 85 and 87, SEQ ID NO: 86 and 87, SEQ ID NO: 88 and 90, SEQ ID NO: 89 and 90, SEQ ID NO: 251 and 252, SEQ ID NO: 253 and 254, SEQ ID NO: 255 and 256, SEQ ID NO: 257 and 258, SEQ ID NO: 259 and 260, SEQ ID NO: 261 and 262, SEQ ID NO: 263 and 264, SEQ ID NO: 265 and 266, SEQ ID NO: 267 and 268, SEQ ID NO: 269 and 270, SEQ ID NO: 271 and 272, SEQ ID NO: 273 and 274, SEQ ID NO: 275 and 276, SEQ ID NO: 277 and 278, SEQ ID NO: 279 and 280, SEQ ID NO: 281 and 282, SEQ ID NO: 283 and 284, SEQ ID NO: 285 and 286, SEQ ID NO: 287 and 288, or SEQ ID NO: 289 and 290, respectively. In some embodiments, the anti-B7H4 antibody comprises a heavy chain variable region and a light chain variable region comprising amino acid sequences that are at least 80%, at least 85%, at least 90%, at least 95%, at least 98%, at least 99%, or at least 100% identical to the amino acid sequences of SEQ ID NO: 83 and 84, SEQ ID NO: 97 and 98, SEQ ID NO: 105 and 106, SEQ ID NO: 113 and 114, SEQ ID NO: 121 and 122, SEQ ID NO: 129 and 130, SEQ ID NO: 137 and 138, SEQ ID NO: 153 and 154, SEQ ID NO: 161 and 162, SEQ ID NO: 169 and 170, SEQ ID NO: 177 and 178, SEQ ID NO: 185 and 186, SEQ ID NO: 193 and 194, SEQ ID NO: 201 and 202, SEQ ID NO: 209 and 210, SEQ ID NO: 217 and 218, SEQ ID NO: 225 and 226, SEQ ID NO: 233 and 234, SEQ ID NO: 241 and 242, or SEQ ID NO: 249 and 250, respectively.

In some embodiments, the L moiety of compound 3.1 comprises an antibody that binds to CD70. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 291, 292, 293, 294, 295, and 296, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 291, 292, 293, 294, 295, and 296, respectively. In some embodiments, the anti-CD70 antibody comprises a heavy chain variable region comprising an amino acid sequence that is at least 80%, at least 85%, at least 90%, at least 95%, at least 98%, or 99% identical to the amino acid sequence of SEQ ID NO: 297 and a light chain variable region comprising an amino acid sequence that is at least 80% at least 85%, at least 90%, at least 95%, at least 98%, or 99% identical to the amino acid sequence of SEQ ID NO: 298. In some embodiments, the anti-CD70 antibody comprises a heavy chain comprising an amino acid sequence that is at least 80%, at least 85%, at least 90%, at least 95%, at least 98%, or 99% identical to the amino acid sequence of SEQ ID NO: 299 and a light chain comprising an amino acid sequence that is at least 80% at least 85%, at least 90%, at least 95%, at least 98%, or 99% identical to the amino acid sequence of SEQ ID NO: 300.

In some embodiments, the L moiety of compound 3.1 comprises an antibody that binds to TROP2. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 301, 302, 303, 304, 305, and 306, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 301, 302, 303, 304, 305, and 306, respectively. In some cases, an antibody provided herein binds to TROP2. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 309, 310, 311, 312, 313, and 314 respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 309, 310, 311, 312, 313, and 314, respectively. In some embodiments, the anti-TROP2 antibody comprises a heavy chain variable region comprising an amino acid sequence that is at least 80%, at least 85%, at least 90%, at least 95%, at least 98%, or 99% identical to the amino acid sequence of SEQ ID NO: 307 and a light chain variable region comprising an amino acid sequence that is at least 80% at least 85%, at least 90%, at least 95%, at least 98%, or 99% identical to the amino acid sequence of SEQ ID NO: 308. In some embodiments, the anti-TROP2 antibody comprises a heavy chain variable region comprising an amino acid sequence that is at least 80%, at least 85%, at least 90%, at least 95%, at least 98%, or 99% identical to the amino acid sequence of SEQ ID NO: 315 and a light chain variable region comprising an amino acid sequence that is at least 80% at least 85%, at least 90%, at least 95%, at least 98%, or 99% identical to the amino acid sequence of SEQ ID NO: 316.

In some embodiments, the L moiety of compound 3.1 comprises an antibody that binds to MICA. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 317, 318, 319, 320, 321, and 322, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 317, 318, 319, 320, 321, and 322, respectively. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 325, 326, 327, 328, 329, and 330, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 325, 326, 327, 328, 329, and 330, respectively. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 333, 334, 335, 336, 337, and 338, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 333, 334, 335, 336, 337, and 338, respectively. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 341, 342, 343, 344, 345, and 346, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 341, 342, 343, 344, 345, and 346, respectively. In some embodiments, the anti-MICA antibody comprises a heavy chain variable region comprising an amino acid sequence that is at least 80%, at least 85%, at least 90%, at least 95%, at least 98%, or 99% identical to the amino acid sequence of SEQ ID NO: 323 and a light chain variable region comprising an amino acid sequence that is at least 80% at least 85%, at least 90%, at least 95%, at least 98%, or 99% identical to the amino acid sequence of SEQ ID NO: 324. In some embodiments, the anti-MICA antibody comprises a heavy chain variable region comprising an amino acid sequence that is at least 80%, at least 85%, at least 90%, at least 95%, at least 98%, or 99% identical to the amino acid sequence of SEQ ID NO: 331 and a light chain variable region comprising an amino acid sequence that is at least 80% at least 85%, at least 90%, at least 95%, at least 98%, or 99% identical to the amino acid sequence of SEQ ID NO: 332. In some embodiments, the anti-MICA antibody comprises a heavy chain variable region comprising an amino acid sequence that is at least 80%, at least 85%, at least 90%, at least 95%, at least 98%, or 99% identical to the amino acid sequence of SEQ ID NO: 340 and a light chain variable region comprising an amino acid sequence that is at least 80% at least 85%, at least 90%, at least 95%, at least 98%, or 99% identical to the amino acid sequence of SEQ ID NO: 340. In some embodiments, the anti-MICA antibody comprises a heavy chain variable region comprising an amino acid sequence that is at least 80%, at least 85%, at least 90%, at least 95%, at least 98%, or 99% identical to the amino acid sequence of SEQ ID NO: 347 and a light chain variable region comprising an amino acid sequence that is at least 80% at least 85%, at least 90%, at least 95%, at least 98%, or 99% identical to the amino acid sequence of SEQ ID NO: 348.

In some embodiments, the L moiety of compound 3.1 comprises an antibody that binds to CD51. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 349, 350, 351, 352, 353, and 354, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 349, 350, 351, 352, 353, and 354, respectively. In some cases, an antibody provided herein binds to CD51. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 357, 358, 359, 360, 361, and 362, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 357, 358, 359, 360, 361, and 362, respectively. In some embodiments, the anti-CD51 antibody comprises a heavy chain variable region comprising an amino acid sequence that is at least 80%, at least 85%, at least 90%, at least 95%, at least 98%, or 99% identical to the amino acid sequence of SEQ ID NO: 355 and a light chain variable region comprising an amino acid sequence that is at least 80% at least 85%, at least 90%, at least 95%, at least 98%, or 99% identical to the amino acid sequence of SEQ ID NO: 356. In some embodiments, the anti-CD51 antibody comprises a heavy chain variable region comprising an amino acid sequence that is at least 80%, at least 85%, at least 90%, at least 95%, at least 98%, or 99% identical to the amino acid sequence of SEQ ID NO: 363 and a light chain variable region comprising an amino acid sequence that is at least 80% at least 85%, at least 90%, at least 95%, at least 98%, or 99% identical to the amino acid sequence of SEQ ID NO: 364.

In some embodiments, the L moiety of compound 3.1 comprises an antibody that binds to gpA33. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 365, 366, 367, 368, 369, and 370, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 365, 366, 367, 368, 369, and 370, respectively. In some embodiments, the anti-gpA33 antibody comprises a heavy chain variable region comprising an amino acid sequence that is at least 80%, at least 85%, at least 90%, at least 95%, at least 98%, or 99% identical to the amino acid sequence of SEQ ID NO: 371 and a light chain variable region comprising an amino acid sequence that is at least 80% at least 85%, at least 90%, at least 95%, at least 98%, or 99% identical to the amino acid sequence of SEQ ID NO: 372.

In some embodiments, the L moiety of compound 3.1 comprises an antibody that binds to IL1Rap. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 373, 374, 375, 376, 377, and 378, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 373, 374, 375, 376, 377, and 378, respectively. In some embodiments, the anti-IL1Rap antibody comprises a heavy chain variable region comprising an amino acid sequence that is at least 80%, at least 85%, at least 90%, at least 95%, at least 98%, or 99% identical to the amino acid sequence of SEQ ID NO: 379 and a light chain variable region comprising an amino acid sequence that is at least 80% at least 85%, at least 90%, at least 95%, at least 98%, or 99% identical to the amino acid sequence of SEQ ID NO: 380.

In some embodiments, the L moiety of compound 3.1 comprises an antibody that binds to EpCAM. In some cases, the antibody comprises a set of CDR sequences (CDR-H1, CDR-H2, CDR-H3, CDR-L1, CDR-L2, and CDR-L3, respectively) of which each sequence comprises at least 80% sequence identity, at least 85% sequence identity, at least 90% sequence identity, at least 95%, or 100% sequence identity to amino acid sequences from a set of amino acid sequences selected from the group consisting of SEQ ID NOs: 381-386, SEQ ID NOs: 389-394, SEQ ID NOs: 397-402, SEQ ID NOs: 405-410, SEQ ID NOs: 413-418, or SEQ ID NOs: 421-426. In some embodiments, the anti-EpCAM antibody comprises a heavy chain variable region and a light chain variable region comprising amino acid sequences that are at least 80%, at least 85%, at least 90%, at least 95%, at least 98%, at least 99%, or at least 100% identical to the amino acid sequences of SEQ ID NO: 387 and 388, SEQ ID NO: 395 and 396, SEQ ID NO: 403 and 404, SEQ ID NO: 411 and 412, SEQ ID NO: 419 and 420, or SEQ ID NO: 427 and 428, respectively.

In some embodiments, the L moiety of compound 3.1 comprises an antibody that binds to CD352. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 429, 430, 431, 432, 433, and 434, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 429, 430, 431, 432, 433, and 434, respectively. In some embodiments, the anti-CD352 antibody comprises a heavy chain variable region comprising an amino acid sequence that is at least 80%, at least 85%, at least 90%, at least 95%, at least 98%, or 99% identical to the amino acid sequence of SEQ ID NO: 435 and a light chain variable region comprising an amino acid sequence that is at least 80% at least 85%, at least 90%, at least 95%, at least 98%, or 99% identical to the amino acid sequence of SEQ ID NO: 436.

In some embodiments, the L moiety of compound 3.1 comprises an antibody that binds to CS1. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 437, 438, 439, 440, 441, and 442, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 437, 438, 439, 440, 441, and 442, respectively. In some embodiments, the anti-CS1 antibody comprises a heavy chain variable region comprising an amino acid sequence that is at least 80%, at least 85%, at least 90%, at least 95%, at least 98%, or 99% identical to the amino acid sequence of SEQ ID NO: 443 and a light chain variable region comprising an amino acid sequence that is at least 80% at least 85%, at least 90%, at least 95%, at least 98%, or 99% identical to the amino acid sequence of SEQ ID NO: 444.

In some embodiments, the L moiety of compound 3.1 comprises an antibody that binds to CD38. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 445, 446, 447, 448, 449, and 450, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 445, 446, 447, 448, 449, and 450, respectively. In some embodiments, the anti-CD38 antibody comprises a heavy chain variable region comprising an amino acid sequence that is at least 80%, at least 85%, at least 90%, at least 95%, at least 98%, or 99% identical to the amino acid sequence of SEQ ID NO: 451 and a light chain variable region comprising an amino acid sequence that is at least 80% at least 85%, at least 90%, at least 95%, at least 98%, or 99% identical to the amino acid sequence of SEQ ID NO: 452.

In some embodiments, the L moiety of compound 3.1 comprises an antibody that binds to CD25. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 453, 454, 455, 456, 457, and 458, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 453, 454, 455, 456, 457, and 458, respectively. In some embodiments, the anti-CD25 antibody comprises a heavy chain variable region comprising an amino acid sequence that is at least 80%, at least 85%, at least 90%, at least 95%, at least 98%, or 99% identical to the amino acid sequence of SEQ ID NO: 459 and a light chain variable region comprising an amino acid sequence that is at least 80% at least 85%, at least 90%, at least 95%, at least 98%, or 99% identical to the amino acid sequence of SEQ ID NO: 460.

In some embodiments, the L moiety of compound 3.1 comprises an antibody that binds to ADAM9. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 461, 462, 463, 464, 465, and 466, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 461, 462, 463, 464, 465, and 466, respectively. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 469, 470, 471, 472, 473, and 474, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 469, 470, 471, 472, 473, and 474, respectively. In some embodiments, the anti-ADAM9 antibody comprises a heavy chain variable region comprising an amino acid sequence that is at least 80%, at least 85%, at least 90%, at least 95%, at least 98%, or 99% identical to the amino acid sequence of SEQ ID NO: 467 and a light chain variable region comprising an amino acid sequence that is at least 80% at least 85%, at least 90%, at least 95%, at least 98%, or 99% identical to the amino acid sequence of SEQ ID NO: 468. In some embodiments, the anti-ADAM9 antibody comprises a heavy chain variable region comprising an amino acid sequence that is at least 80%, at least 85%, at least 90%, at least 95%, at least 98%, or 99% identical to the amino acid sequence of SEQ ID NO: 475 and a light chain variable region comprising an amino acid sequence that is at least 80% at least 85%, at least 90%, at least 95%, at least 98%, or 99% identical to the amino acid sequence of SEQ ID NO: 476.

In some embodiments, the L moiety of compound 3.1 comprises an antibody that binds to CD59. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 477, 478, 479, 480, 481, and 482, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 477, 478, 479, 480, 481, and 482, respectively. In some embodiments, the anti-CD59 antibody comprises a heavy chain variable region comprising an amino acid sequence that is at least 80%, at least 85%, at least 90%, at least 95%, at least 98%, or 99% identical to the amino acid sequence of SEQ ID NO: 483 and a light chain variable region comprising an amino acid sequence that is at least 80% at least 85%, at least 90%, at least 95%, at least 98%, or 99% identical to the amino acid sequence of SEQ ID NO: 484.

In some embodiments, the L moiety of compound 3.1 comprises an antibody that binds to CD19. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 485, 486, 487, 488, 489, and 490, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 485, 486, 487, 488, 489, and 490, respectively. In some embodiments, the anti-CD19 antibody comprises a heavy chain variable region comprising an amino acid sequence that is at least 80%, at least 85%, at least 90%, at least 95%, at least 98%, or 99% identical to the amino acid sequence of SEQ ID NO: 491 and a light chain variable region comprising an amino acid sequence that is at least 80% at least 85%, at least 90%, at least 95%, at least 98%, or 99% identical to the amino acid sequence of SEQ ID NO: 492. In some embodiments, the anti-CD19 antibody comprises a heavy chain comprising an amino acid sequence that is at least 80%, at least 85%, at least 90%, at least 95%, at least 98%, or 99% identical to the amino acid sequence of SEQ ID NO: 493 and a light chain comprising an amino acid sequence that is at least 80% at least 85%, at least 90%, at least 95%, at least 98%, or 99% identical to the amino acid sequence of SEQ ID NO: 494.

In some embodiments, the L moiety of compound 3.1 comprises an antibody that binds to CD138. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 495, 496, 497, 498, 499, and 500, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 495, 496, 497, 498, 499, and 500, respectively. In some embodiments, the anti-CD138 antibody comprises a heavy chain variable region comprising an amino acid sequence that is at least 80%, at least 85%, at least 90%, at least 95%, at least 98%, or 99% identical to the amino acid sequence of SEQ ID NO: 501 and a light chain variable region comprising an amino acid sequence that is at least 80% at least 85%, at least 90%, at least 95%, at least 98%, or 99% identical to the amino acid sequence of SEQ ID NO: 502.

In some embodiments, the L moiety of compound 3.1 comprises an antibody that binds to CD166. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 503, 504, 505, 506, 507, and 508, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 503, 504, 505, 506, 507, and 508, respectively. In some embodiments, the anti-CD166 antibody comprises a heavy chain variable region comprising an amino acid sequence that is at least 80%, at least 85%, at least 90%, at least 95%, at least 98%, or 99% identical to the amino acid sequence of SEQ ID NO: 509 and a light chain variable region comprising an amino acid sequence that is at least 80% at least 85%, at least 90%, at least 95%, at least 98%, or 99% identical to the amino acid sequence of SEQ ID NO: 510.

In some embodiments, the L moiety of compound 3.1 comprises an antibody that binds to CD56. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 511, 512, 513, 514, 515, and 516, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 511, 512, 513, 514, 515, and 516, respectively. In some embodiments, the anti-CD56 antibody comprises a heavy chain variable region comprising an amino acid sequence that is at least 80%, at least 85%, at least 90%, at least 95%, at least 98%, or 99% identical to the amino acid sequence of SEQ ID NO: 517 and a light chain variable region comprising an amino acid sequence that is at least 80% at least 85%, at least 90%, at least 95%, at least 98%, or 99% identical to the amino acid sequence of SEQ ID NO: 518.

In some embodiments, the L moiety of compound 3.1 comprises an antibody that binds to CD74. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 519, 520, 521, 522, 523, and 524, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 519, 520, 521, 522, 523, and 524, respectively. In some embodiments, the anti-CD74 antibody comprises a heavy chain variable region comprising an amino acid sequence that is at least 80%, at least 85%, at least 90%, at least 95%, at least 98%, or 99% identical to the amino acid sequence of SEQ ID NO: 525 and a light chain variable region comprising an amino acid sequence that is at least 80% at least 85%, at least 90%, at least 95%, at least 98%, or 99% identical to the amino acid sequence of SEQ ID NO: 526.

In some embodiments, the L moiety of compound 3.1 comprises an antibody that binds to CEACAM5. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 527, 528, 529, 530, 531, and 532, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 527, 528, 529, 530, 531, and 532, respectively. In some embodiments, the anti-CEACAM5 antibody comprises a heavy chain variable region comprising an amino acid sequence that is at least 80%, at least 85%, at least 90%, at least 95%, at least 98%, or 99% identical to the amino acid sequence of SEQ ID NO: 533 and a light chain variable region comprising an amino acid sequence that is at least 80% at least 85%, at least 90%, at least 95%, at least 98%, or 99% identical to the amino acid sequence of SEQ ID NO: 534.

In some embodiments, the L moiety of compound 3.1 comprises an antibody that binds to CanAg. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 535, 536, 537, 538, 539, and 540, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 535, 536, 537, 538, 539, and 540, respectively. In some embodiments, the anti-CanAg antibody comprises a heavy chain variable region comprising an amino acid sequence that is at least 80%, at least 85%, at least 90%, at least 95%, at least 98%, or 99% identical to the amino acid sequence of SEQ ID NO: 541 and a light chain variable region comprising an amino acid sequence that is at least 80% at least 85%, at least 90%, at least 95%, at least 98%, or 99% identical to the amino acid sequence of SEQ ID NO: 542.

In some embodiments, the L moiety of compound 3.1 comprises an antibody that binds to DLL-3. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 543, 544, 545, 546, 547, and 548, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 543, 544, 545, 546, 547, and 548, respectively. In some embodiments, the anti-DLL-3 antibody comprises a heavy chain variable region comprising an amino acid sequence that is at least 80%, at least 85%, at least 90%, at least 95%, at least 98%, or 99% identical to the amino acid sequence of SEQ ID NO: 549 and a light chain variable region comprising an amino acid sequence that is at least 80% at least 85%, at least 90%, at least 95%, at least 98%, or 99% identical to the amino acid sequence of SEQ ID NO: 550.

In some embodiments, the L moiety of compound 3.1 comprises an antibody that binds to DPEP-3. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 551, 552, 553, 554, 555, and 556, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 551, 552, 553, 554, 555, and 556, respectively. In some embodiments, the anti-DPEP-3 antibody comprises a heavy chain variable region comprising an amino acid sequence that is at least 80%, at least 85%, at least 90%, at least 95%, at least 98%, or 99% identical to the amino acid sequence of SEQ ID NO: 557 and a light chain variable region comprising an amino acid sequence that is at least 80% at least 85%, at least 90%, at least 95%, at least 98%, or 99% identical to the amino acid sequence of SEQ ID NO: 558.

In some embodiments, the L moiety of compound 3.1 comprises an antibody that binds to EGFR. In some cases, the antibody comprises a set of CDR sequences (CDR-H1, CDR-H2, CDR-H3, CDR-L1, CDR-L2, and CDR-L3, respectively) of which each sequence comprises at least 80% sequence identity, at least 85% sequence identity, at least 90% sequence identity, at least 95%, or 100% sequence identity to amino acid sequences from a set of amino acid sequences selected from the group consisting of SEQ ID NOs: 559-564, SEQ ID NOs: 567-572, SEQ ID NOs: 575-580, and SEQ ID NOs: 895-900. In some embodiments, the anti-EGFR antibody comprises a heavy chain variable region and a light chain variable region comprising amino acid sequences that are at least 80%, at least 85%, at least 90%, at least 95%, at least 98%, at least 99%, or at least 100% identical to the amino acid sequences of SEQ ID NO: 565 and 566, SEQ ID NO: 573 and 574, SEQ ID NO: 581 and 582, or SEQ ID NO: 901 and 902, respectively.

In some embodiments, the L moiety of compound 3.1 comprises an antibody that binds to FRa. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 583, 584, 585, 586, 587, and 588, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 583, 584, 585, 586, 587, and 588, respectively. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 591, 592, 593, 594, 595, and 596, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 591, 592, 593, 594, 595, and 596, respectively. In some embodiments, the anti-FRa antibody comprises a heavy chain variable region comprising an amino acid sequence that is at least 80%, at least 85%, at least 90%, at least 95%, at least 98%, or 99% identical to the amino acid sequence of SEQ ID NO: 589 and a light chain variable region comprising an amino acid sequence that is at least 80% at least 85%, at least 90%, at least 95%, at least 98%, or 99% identical to the amino acid sequence of SEQ ID NO: 590. In some embodiments, the anti-FRa antibody comprises a heavy chain variable region comprising an amino acid sequence that is at least 80%, at least 85%, at least 90%, at least 95%, at least 98%, or 99% identical to the amino acid sequence of SEQ ID NO: 597 and a light chain variable region comprising an amino acid sequence that is at least 80% at least 85%, at least 90%, at least 95%, at least 98%, or 99% identical to the amino acid sequence of SEQ ID NO: 598.

In some embodiments, the L moiety of compound 3.1 comprises an antibody that binds to MUC-1. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 599, 600, 601, 602, 603, and 604, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 599, 600, 601, 602, 603, and 604, respectively. In some embodiments, the anti-MUC-1 antibody comprises a heavy chain variable region comprising an amino acid sequence that is at least 80%, at least 85%, at least 90%, at least 95%, at least 98%, or 99% identical to the amino acid sequence of SEQ ID NO: 605 and a light chain variable region comprising an amino acid sequence that is at least 80% at least 85%, at least 90%, at least 95%, at least 98%, or 99% identical to the amino acid sequence of SEQ ID NO: 606.

In some embodiments, the L moiety of compound 3.1 comprises an antibody that binds to Mesothelin. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 607, 608, 609, 610, 611, and 612, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 607, 608, 609, 610, 611, and 612, respectively. In some embodiments, the anti-Mesothelin antibody comprises a heavy chain variable region comprising an amino acid sequence that is at least 80%, at least 85%, at least 90%, at least 95%, at least 98%, or 99% identical to the amino acid sequence of SEQ ID NO: 613 and a light chain variable region comprising an amino acid sequence that is at least 80% at least 85%, at least 90%, at least 95%, at least 98%, or 99% identical to the amino acid sequence of SEQ ID NO: 614.

In some embodiments, the L moiety of compound 3.1 comprises an antibody that binds to ROR-1. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 615, 616, 617, 618, 619, and 620, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 615, 616, 617, 618, 619, and 620, respectively. In some embodiments, the anti-ROR-1 antibody comprises a heavy chain variable region comprising an amino acid sequence that is at least 80%, at least 85%, at least 90%, at least 95%, at least 98%, or 99% identical to the amino acid sequence of SEQ ID NO: 621 and a light chain variable region comprising an amino acid sequence that is at least 80% at least 85%, at least 90%, at least 95%, at least 98%, or 99% identical to the amino acid sequence of SEQ ID NO: 622.

In some embodiments, the L moiety of compound 3.1 comprises an antibody that binds to B7H3. In some cases, the antibody comprises a set of CDR sequences (CDR-H1, CDR-H2, CDR-H3, CDR-L1, CDR-L2, and CDR-L3, respectively) of which each sequence comprises at least 80% sequence identity, at least 85% sequence identity, at least 90% sequence identity, at least 95%, or 100% sequence identity to amino acid sequences from a set of amino acid sequences selected from the group consisting of SEQ ID NOs: 623-628, SEQ ID NOs: 631-636, SEQ ID NOs: 639-644, SEQ ID NOs: 647-652, SEQ ID NOs: 655-660, SEQ ID NOs: 663-668, SEQ ID NOs: 671-676, SEQ ID NOs: 679-684, SEQ ID NOs: 687-692, SEQ ID NOs: 695-700, and SEQ ID NOs: 705-710. In some embodiments, the anti-B7H3 antibody comprises a heavy chain variable region and a light chain variable region comprising amino acid sequences that are at least 80%, at least 85%, at least 90%, at least 95%, at least 98%, at least 99%, or at least 100% identical to the amino acid sequences of SEQ ID NO: 629 and 630, SEQ ID NOs: 637 and 638, SEQ ID NOs: 645 and 646, SEQ ID NOs: 653 and 654, SEQ ID NOs: 661 and 662, SEQ ID NOs: 669 and 670, SEQ ID NOs: 677 and 678, SEQ ID NOs: 685 and 686, SEQ ID NOs: 693 and 694, SEQ ID NOs: 701 and 702, SEQ ID NOs: 703 and 704, SEQ ID NOs: 711 and 712, SEQ ID NOs: 713 and 714, and SEQ ID NOs: 715 and 716, respectively.

In some embodiments, the L moiety of compound 3.1 comprises an antibody that binds to HER3. In some embodiments, the anti-HER3 antibody comprises a heavy chain comprising an amino acid sequence that is at least 80%, at least 85%, at least 90%, at least 95%, at least 98%, or 99% identical to the amino acid sequence of SEQ ID NO: 717 and a light chain comprising an amino acid sequence that is at least 80% at least 85%, at least 90%, at least 95%, at least 98%, or 99% identical to the amino acid sequence of SEQ ID NO: 718. In some cases, an antibody provided herein binds to HER3. In some embodiments, the anti-HER3 antibody comprises a heavy chain comprising an amino acid sequence that is at least 80%, at least 85%, at least 90%, at least 95%, at least 98%, or 99% identical to the amino acid sequence of SEQ ID NO: 719 and a light chain comprising an amino acid sequence that is at least 80% at least 85%, at least 90%, at least 95%, at least 98%, or 99% identical to the amino acid sequence of SEQ ID NO: 720. In some cases, an antibody provided herein binds to HER3. In some embodiments, the anti-HER3 antibody comprises a heavy chain comprising an amino acid sequence that is at least 80%, at least 85%, at least 90%, at least 95%, at least 98%, or 99% identical to the amino acid sequence of SEQ ID NO: 721 and a light chain comprising an amino acid sequence that is at least 80% at least 85%, at least 90%, at least 95%, at least 98%, or 99% identical to the amino acid sequence of SEQ ID NO: 722. In some cases, an antibody provided herein binds to HER3. In some embodiments, the anti-HER3 antibody comprises a heavy chain comprising an amino acid sequence that is at least 80%, at least 85%, at least 90%, at least 95%, at least 98%, or 99% identical to the amino acid sequence of SEQ ID NO: 723 and a light chain comprising an amino acid sequence that is at least 80% at least 85%, at least 90%, at least 95%, at least 98%, or 99% identical to the amino acid sequence of SEQ ID NO: 724.

In some embodiments, the L moiety of compound 3.1 comprises an antibody that binds to PTK7. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 725, 726, 727, 728, 729, and 730, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 725, 726, 727, 728, 729, and 730, respectively. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 733, 734, 735, 736, 737, and 738, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 733, 734, 735, 736, 737, and 738, respectively. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 741, 742, 743, 744, 745, and 746, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 741, 742, 743, 744, 745, and 746, respectively. In some cases, the anti-PTK7 antibody comprises a heavy chain comprising an amino acid sequence that is at least 80%, at least 85%, at least 90%, at least 95%, at least 98%, or 99% identical to the amino acid sequence of SEQ ID NO: 731 and a light chain comprising an amino acid sequence that is at least 80% at least 85%, at least 90%, at least 95%, at least 98%, or 99% identical to the amino acid sequence of SEQ ID NO: 732. In some cases, the anti-PTK7 antibody comprises a heavy chain comprising an amino acid sequence that is at least 80%, at least 85%, at least 90%, at least 95%, at least 98%, or 99% identical to the amino acid sequence of SEQ ID NO: 739 and a light chain comprising an amino acid sequence that is at least 80% at least 85%, at least 90%, at least 95%, at least 98%, or 99% identical to the amino acid sequence of SEQ ID NO: 740. In some cases, the anti-PTK7 antibody comprises a heavy chain comprising an amino acid sequence that is at least 80%, at least 85%, at least 90%, at least 95%, at least 98%, or 99% identical to the amino acid sequence of SEQ ID NO: 747 and a light chain comprising an amino acid sequence that is at least 80% at least 85%, at least 90%, at least 95%, at least 98%, or 99% identical to the amino acid sequence of SEQ ID NO: 748.

In some embodiments, the L moiety of compound 3.1 comprises an antibody that binds to ZIP6. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 749, 750, 751, 752, 753, and 754, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 749, 750, 751, 752, 753, and 754, respectively. In some cases, the anti-ZIP6 antibody comprises a heavy chain variable region comprising an amino acid sequence that is at least 80%, at least 85%, at least 90%, at least 95%, at least 98%, or 99% identical to the amino acid sequence of SEQ ID NO: 755 and a light chain variable region comprising an amino acid sequence that is at least 80% at least 85%, at least 90%, at least 95%, at least 98%, or 99% identical to the amino acid sequence of SEQ ID NO: 756. In some embodiments, the anti-ZIP6 antibody comprises a heavy chain comprising an amino acid sequence that is at least 80%, at least 85%, at least 90%, at least 95%, at least 98%, or 99% identical to the amino acid sequence of SEQ ID NO: 757 and a light chain comprising an amino acid sequence that is at least 80% at least 85%, at least 90%, at least 95%, at least 98%, or 99% identical to the amino acid sequence of SEQ ID NO: 758.

In some embodiments, the L moiety of compound 3.1 comprises an antibody that binds to Integrin αvβ6. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 759, 760, 761, 762, 763, and 764, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 759, 760, 761, 762, 763, and 764, respectively. In some embodiments, the anti-Integrin αvβ6 antibody comprises a heavy chain variable region comprising an amino acid sequence that is at least 80%, at least 85%, at least 90%, at least 95%, at least 98%, or 99% identical to the amino acid sequence of SEQ ID NO: 765 and a light chain variable region comprising an amino acid sequence that is at least 80% at least 85%, at least 90%, at least 95%, at least 98%, or 99% identical to the amino acid sequence of SEQ ID NO: 766.

In some embodiments, the L moiety of compound 3.1 comprises an antibody that binds to CD48. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 767, 768, 769, 770, 771, and 772, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 767, 768, 769, 770, 771, and 772, respectively. In some embodiments, the anti-CD48 antibody comprises a heavy chain variable region comprising an amino acid sequence that is at least 80%, at least 85%, at least 90%, at least 95%, at least 98%, or 99% identical to the amino acid sequence of SEQ ID NO: 773 and a light chain variable region comprising an amino acid sequence that is at least 80% at least 85%, at least 90%, at least 95%, at least 98%, or 99% identical to the amino acid sequence of SEQ ID NO: 774.

In some embodiments, the L moiety of compound 3.1 comprises an antibody that binds to IGF-1R. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 775, 776, 777, 778, 779, and 780, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 775, 776, 777, 778, 779, and 780, respectively. In some embodiments, the anti-IGF-1R antibody comprises a heavy chain variable region comprising an amino acid sequence that is at least 80%, at least 85%, at least 90%, at least 95%, at least 98%, or 99% identical to the amino acid sequence of SEQ ID NO: 781 and a light chain variable region comprising an amino acid sequence that is at least 80% at least 85%, at least 90%, at least 95%, at least 98%, or 99% identical to the amino acid sequence of SEQ ID NO: 782.

In some embodiments, the L moiety of compound 3.1 comprises an antibody that binds to Claudin-18.2. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 783, 784, 785, 786, 787, and 788, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 783, 784, 785, 786, 787, and 788, respectively. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 791, 792, 793, 794, 795, and 796, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 791, 792, 793, 794, 795, and 796, respectively. In some embodiments, the anti-Claudin-18.2 antibody comprises a heavy chain variable region comprising an amino acid sequence that is at least 80%, at least 85%, at least 90%, at least 95%, at least 98%, or 99% identical to the amino acid sequence of SEQ ID NO: 789 and a light chain variable region comprising an amino acid sequence that is at least 80% at least 85%, at least 90%, at least 95%, at least 98%, or 99% identical to the amino acid sequence of SEQ ID NO: 790. In some embodiments, the anti-Claudin-18.2 antibody comprises a heavy chain variable region comprising an amino acid sequence that is at least 80%, at least 85%, at least 90%, at least 95%, at least 98%, or 99% identical to the amino acid sequence of SEQ ID NO: 797 and a light chain variable region comprising an amino acid sequence that is at least 80% at least 85%, at least 90%, at least 95%, at least 98%, or 99% identical to the amino acid sequence of SEQ ID NO: 798.

In some embodiments, the L moiety of compound 3.1 comprises an antibody that binds to Nectin-4. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 799, 800, 801, 802, 803, and 804, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 799, 800, 801, 802, 803, and 804, respectively. In some embodiments, the anti-Nectin-4 antibody comprises a heavy chain variable region comprising an amino acid sequence that is at least 80%, at least 85%, at least 90%, at least 95%, at least 98%, or 99% identical to the amino acid sequence of SEQ ID NO: 805 and a light chain variable region comprising an amino acid sequence that is at least 80% at least 85%, at least 90%, at least 95%, at least 98%, or 99% identical to the amino acid sequence of SEQ ID NO: 806.

In some embodiments, the L moiety of compound 3.1 comprises an antibody that binds to SLTRK6. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 807, 808, 809, 810, 811, and 812, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 807, 808, 809, 810, 811, and 812, respectively. In some embodiments, the anti-SLTRK6 antibody comprises a heavy chain variable region comprising an amino acid sequence that is at least 80%, at least 85%, at least 90%, at least 95%, at least 98%, or 99% identical to the amino acid sequence of SEQ ID NO: 813 and a light chain variable region comprising an amino acid sequence that is at least 80% at least 85%, at least 90%, at least 95%, at least 98%, or 99% identical to the amino acid sequence of SEQ ID NO: 814.

In some embodiments, the L moiety of compound 3.1 comprises an antibody that binds to CD142. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 815, 816, 817, 818, 819, and 820, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 815, 816, 817, 818, 819, and 820, respectively. In some embodiments, the anti-CD142 antibody comprises a heavy chain variable region comprising an amino acid sequence that is at least 80%, at least 85%, at least 90%, at least 95%, at least 98%, or 99% identical to the amino acid sequence of SEQ ID NO: 821 and a light chain variable region comprising an amino acid sequence that is at least 80% at least 85%, at least 90%, at least 95%, at least 98%, or 99% identical to the amino acid sequence of SEQ ID NO: 822.

In some embodiments, the L moiety of compound 3.1 comprises an antibody that binds to Sialyl-Thomsen-nouveau antigen (STn). In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 823, 824, 825, 826, 827, and 828, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 823, 824, 825, 826, 827, and 828, respectively.

In some embodiments, the anti-STn antibody comprises a heavy chain variable region comprising an amino acid sequence that is at least 80%, at least 85%, at least 90%, at least 95%, at least 98%, or 99% identical to the amino acid sequence of SEQ ID NO: 829 and a light chain variable region comprising an amino acid sequence that is at least 80% at least 85%, at least 90%, at least 95%, at least 98%, or 99% identical to the amino acid sequence of SEQ ID NO: 830.

In some embodiments, the L moiety of compound 3.1 comprises an antibody that binds to CD20. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 831, 832, 833, 834, 835, and 836, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 831, 832, 833, 834, 835, and 836, respectively. In some embodiments, the anti-CD20 antibody comprises a heavy chain variable region comprising an amino acid sequence that is at least 80%, at least 85%, at least 90%, at least 95%, at least 98%, or 99% identical to the amino acid sequence of SEQ ID NO: 837 and a light chain variable region comprising an amino acid sequence that is at least 80% at least 85%, at least 90%, at least 95%, at least 98%, or 99% identical to the amino acid sequence of SEQ ID NO: 838.

In some embodiments, the L moiety of compound 3.1 comprises an antibody that binds to HER2. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 839, 840, 841, 842, 843, and 844, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 839, 840, 841, 842, 843, and 844, respectively. In some embodiments, the anti-HER2 antibody comprises a heavy chain variable region comprising an amino acid sequence that is at least 80%, at least 85%, at least 90%, at least 95%, at least 98%, or 99% identical to the amino acid sequence of SEQ ID NO: 845 and a light chain variable region comprising an amino acid sequence that is at least 80% at least 85%, at least 90%, at least 95%, at least 98%, or 99% identical to the amino acid sequence of SEQ ID NO: 846.

In some embodiments, the L moiety of compound 3.1 comprises an antibody that binds to CD79b. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 847, 848, 849, 850, 851, and 852, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 847, 848, 849, 850, 851, and 852, respectively. In some embodiments, the anti-CD79b antibody comprises a heavy chain variable region comprising an amino acid sequence that is at least 80%, at least 85%, at least 90%, at least 95%, at least 98%, or 99% identical to the amino acid sequence of SEQ ID NO: 853 and a light chain variable region comprising an amino acid sequence that is at least 80% at least 85%, at least 90%, at least 95%, at least 98%, or 99% identical to the amino acid sequence of SEQ ID NO: 854.

In some embodiments, the L moiety of compound 3.1 comprises an antibody that binds to NaPi2B. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 855, 856, 857, 858, 859, and 860, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 855, 856, 857, 858, 859, and 860, respectively. In some embodiments, the anti-NaPi2B antibody comprises a heavy chain variable region comprising an amino acid sequence that is at least 80%, at least 85%, at least 90%, at least 95%, at least 98%, or 99% identical to the amino acid sequence of SEQ ID NO: 861 and a light chain variable region comprising an amino acid sequence that is at least 80% at least 85%, at least 90%, at least 95%, at least 98%, or 99% identical to the amino acid sequence of SEQ ID NO: 862.

In some embodiments, the L moiety of compound 3.1 comprises an antibody that binds to Muc16. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 863, 864, 865, 866, 867, and 868, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 863, 864, 865, 866, 867, and 868, respectively. In some embodiments, the anti-Muc16 antibody comprises a heavy chain variable region comprising an amino acid sequence that is at least 80%, at least 85%, at least 90%, at least 95%, at least 98%, or 99% identical to the amino acid sequence of SEQ ID NO: 869 and a light chain variable region comprising an amino acid sequence that is at least 80% at least 85%, at least 90%, at least 95%, at least 98%, or 99% identical to the amino acid sequence of SEQ ID NO: 870.

In some embodiments, the L moiety of compound 3.1 comprises an antibody that binds to STEAP1. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 871, 872, 873, 874, 875, and 876, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 871, 872, 873, 874, 875, and 876, respectively. In some embodiments, the anti-STEAP1 antibody comprises a heavy chain variable region comprising an amino acid sequence that is at least 80%, at least 85%, at least 90%, at least 95%, at least 98%, or 99% identical to the amino acid sequence of SEQ ID NO: 877 and a light chain variable region comprising an amino acid sequence that is at least 80% at least 85%, at least 90%, at least 95%, at least 98%, or 99% identical to the amino acid sequence of SEQ ID NO: 878.

In some embodiments, the L moiety of compound 3.1 comprises an antibody that binds to BCMA. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 879, 880, 881, 882, 883, and 884, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 879, 880, 881, 882, 883, and 884, respectively. In some embodiments, the anti-BCMA antibody comprises a heavy chain variable region comprising an amino acid sequence that is at least 80%, at least 85%, at least 90%, at least 95%, at least 98%, or 99% identical to the amino acid sequence of SEQ ID NO: 885 and a light chain variable region comprising an amino acid sequence that is at least 80% at least 85%, at least 90%, at least 95%, at least 98%, or 99% identical to the amino acid sequence of SEQ ID NO: 886.

In some embodiments, the L moiety of compound 3.1 comprises an antibody that binds to c-Met. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 887, 888, 889, 890, 891, and 892, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 887, 888, 889, 890, 891, and 892, respectively. In some embodiments, the anti-c-Met antibody comprises a heavy chain variable region comprising an amino acid sequence that is at least 80%, at least 85%, at least 90%, at least 95%, at least 98%, or 99% identical to the amino acid sequence of SEQ ID NO: 893 and a light chain variable region comprising an amino acid sequence that is at least 80% at least 85%, at least 90%, at least 95%, at least 98%, or 99% identical to the amino acid sequence of SEQ ID NO: 893.

In some embodiments, the L moiety of compound comprises an antibody that binds to SLAMF7. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 903, 904, 905, 906, 907, and 908, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 903, 904, 905, 906, 907, and 908, respectively. In some embodiments, the anti-SLAMF7 antibody comprises a heavy chain variable region comprising an amino acid sequence that is at least 80%, at least 85%, at least 90%, at least 95%, at least 98%, or 99% identical to the amino acid sequence of SEQ ID NO: 909 and a light chain variable region comprising an amino acid sequence that is at least 80% at least 85%, at least 90%, at least 95%, at least 98%, or 99% identical to the amino acid sequence of SEQ ID NO: 910.

In some embodiments, the L moiety of compound 3.1 comprises an antibody that binds to C4.4a. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 911, 912, 913, 914, 915, and 916, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 911, 912, 913, 914, 915, and 916, respectively. In some embodiments, the anti-C4.4a antibody comprises a heavy chain variable region comprising an amino acid sequence that is at least 80%, at least 85%, at least 90%, at least 95%, at least 98%, or 99% identical to the amino acid sequence of SEQ ID NO: 917 and a light chain variable region comprising an amino acid sequence that is at least 80% at least 85%, at least 90%, at least 95%, at least 98%, or 99% identical to the amino acid sequence of SEQ ID NO: 918.

In some embodiments, the L moiety of compound 3.1 comprises an antibody that binds to GCC. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 919, 920, 921, 922, 923, and 924, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 919, 920, 921, 922, 923, and 924, respectively. In some embodiments, the anti-GCC antibody comprises a heavy chain variable region comprising an amino acid sequence that is at least 80%, at least 85%, at least 90%, at least 95%, at least 98%, or 99% identical to the amino acid sequence of SEQ ID NO: 925 and a light chain variable region comprising an amino acid sequence that is at least 80% at least 85%, at least 90%, at least 95%, at least 98%, or 99% identical to the amino acid sequence of SEQ ID NO: 926.

In some embodiments, the L moiety of compound 3.1 comprises an antibody that binds to Ax1. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 927, 928, 929, 930, 931, and 932, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 927, 928, 929, 930, 931, and 932, respectively. In some embodiments, the anti-Ax1 antibody comprises a heavy chain variable region comprising an amino acid sequence that is at least 80%, at least 85%, at least 90%, at least 95%, at least 98%, or 99% identical to the amino acid sequence of SEQ ID NO: 933 and a light chain variable region comprising an amino acid sequence that is at least 80% at least 85%, at least 90%, at least 95%, at least 98%, or 99% identical to the amino acid sequence of SEQ ID NO: 933.

In some embodiments, the L moiety of compound 3.1 comprises an antibody that binds to transmembrane glycoprotein NMB (gpNMB). In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 935, 936, 937, 938, 939, and 940, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 935, 936, 937, 938, 939, and 940, respectively. In some embodiments, the anti-gpNMB antibody comprises a heavy chain variable region comprising an amino acid sequence that is at least 80%, at least 85%, at least 90%, at least 95%, at least 98%, or 99% identical to the amino acid sequence of SEQ ID NO: 941 and a light chain variable region comprising an amino acid sequence that is at least 80% at least 85%, at least 90%, at least 95%, at least 98%, or 99% identical to the amino acid sequence of SEQ ID NO: 942. In some embodiments, the anti-gpNMB antibody comprises a heavy chain variable region comprising an amino acid sequence that is at least 80%, at least 85%, at least 90%, at least 95%, at least 98%, or 99% identical to the amino acid sequence of SEQ ID NO: 943 and a light chain variable region comprising an amino acid sequence that is at least 80% at least 85%, at least 90%, at least 95%, at least 98%, or 99% identical to the amino acid sequence of SEQ ID NO: 944.

In some embodiments, the L moiety of compound 3.1 comprises an antibody that binds to Prolactin receptor. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 945, 946, 947, 948, 949, and 950, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 945, 946, 947, 948, 949, and 950, respectively. In some embodiments, the anti-Prolactin receptor antibody comprises a heavy chain variable region comprising an amino acid sequence that is at least 80%, at least 85%, at least 90%, at least 95%, at least 98%, or 99% identical to the amino acid sequence of SEQ ID NO: 951 and a light chain variable region comprising an amino acid sequence that is at least 80% at least 85%, at least 90%, at least 95%, at least 98%, or 99% identical to the amino acid sequence of SEQ ID NO: 952.

In some embodiments, the L moiety of compound 3.1 comprises an antibody that binds to FGFR2. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 953, 954, 955, 956, 957, and 958, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 953, 954, 955, 956, 957, and 958, respectively. In some embodiments, the anti-FGFR2 antibody comprises a heavy chain variable region comprising an amino acid sequence that is at least 80%, at least 85%, at least 90%, at least 95%, at least 98%, or 99% identical to the amino acid sequence of SEQ ID NO: 959 and a light chain variable region comprising an amino acid sequence that is at least 80% at least 85%, at least 90%, at least 95%, at least 98%, or 99% identical to the amino acid sequence of SEQ ID NO: 960.

In some embodiments, the L moiety of compound 3.1 comprises an antibody that binds to CDCP1. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 961, 962, 963, 964, 965, and 966, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 969, 970, 971, 972, 973, and 974, respectively. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 961, 962, 963, 964, 965, and 966, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 969, 970, 971, 972, 973, and 974, respectively. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 977, 978, 979, 980, 981, and 982, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 977, 978, 979, 980, 981, and 982, respectively. In some cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 985, 986, 987, 988, 989, and 990, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 985, 986, 987, 988, 989, and 990, respectively. In some embodiments, the anti-CDCP1 antibody comprises a heavy chain variable region comprising an amino acid sequence that is at least 80%, at least 85%, at least 90%, at least 95%, at least 98%, or 99% identical to the amino acid sequence of SEQ ID NO: 967 and a light chain variable region comprising an amino acid sequence that is at least 80% at least 85%, at least 90%, at least 95%, at least 98%, or 99% identical to the amino acid sequence of SEQ ID NO: 968. In some embodiments, the anti-CDCP1 antibody comprises a heavy chain variable region comprising an amino acid sequence that is at least 80%, at least 85%, at least 90%, at least 95%, at least 98%, or 99% identical to the amino acid sequence of SEQ ID NO: 975 and a light chain variable region comprising an amino acid sequence that is at least 80% at least 85%, at least 90%, at least 95%, at least 98%, or 99% identical to the amino acid sequence of SEQ ID NO: 976. In some embodiments, the anti-CDCP1 antibody comprises a heavy chain variable region comprising an amino acid sequence that is at least 80%, at least 85%, at least 90%, at least 95%, at least 98%, or 99% identical to the amino acid sequence of SEQ ID NO: 983 and a light chain variable region comprising an amino acid sequence that is at least 80% at least 85%, at least 90%, at least 95%, at least 98%, or 99% identical to the amino acid sequence of SEQ ID NO: 984. In some embodiments, the anti-CDCP1 antibody comprises a heavy chain variable region comprising an amino acid sequence that is at least 80%, at least 85%, at least 90%, at least 95%, at least 98%, or 99% identical to the amino acid sequence of SEQ ID NO: 991 and a light chain variable region comprising an amino acid sequence that is at least 80% at least 85%, at least 90%, at least 95%, at least 98%, or 99% identical to the amino acid sequence of SEQ ID NO: 992.

In some embodiments, the L moiety of compound 3.1 comprises an antibody that binds to ASCT2. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 997, 998, 999, 1000, 1001, and 1002, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 997, 998, 999, 1000, 1001, and 1002, respectively. In some embodiments, the anti-ASCT2 antibody comprises a heavy chain variable region comprising an amino acid sequence that is at least 80%, at least 85%, at least 90%, at least 95%, at least 98%, or 99% identical to the amino acid sequence of SEQ ID NO: 993 and a light chain variable region comprising an amino acid sequence that is at least 80% at least 85%, at least 90%, at least 95%, at least 98%, or 99% identical to the amino acid sequence of SEQ ID NO: 994. In some embodiments, the anti-ASCT2 antibody comprises a heavy chain variable region comprising an amino acid sequence that is at least 80%, at least 85%, at least 90%, at least 95%, at least 98%, or 99% identical to the amino acid sequence of SEQ ID NO: 995 and a light chain variable region comprising an amino acid sequence that is at least 80% at least 85%, at least 90%, at least 95%, at least 98%, or 99% identical to the amino acid sequence of SEQ ID NO: 996. In some embodiments, the anti-ASCT2 antibody comprises a heavy chain variable region comprising an amino acid sequence that is at least 80%, at least 85%, at least 90%, at least 95%, at least 98%, or 99% identical to the amino acid sequence of SEQ ID NO: 1003 and a light chain variable region comprising an amino acid sequence that is at least 80% at least 85%, at least 90%, at least 95%, at least 98%, or 99% identical to the amino acid sequence of SEQ ID NO: 1004.

In some embodiments, the L moiety of compound 3.1 comprises an antibody that binds to CD123. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 1005, 1006, 1007, 1008, 1009, and 1010, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 1005, 1006, 1007, 1008, 1009, and 1010, respectively. In some embodiments, the anti-CD123 antibody comprises a heavy chain variable region comprising an amino acid sequence that is at least 80%, at least 85%, at least 90%, at least 95%, at least 98%, or 99% identical to the amino acid sequence of SEQ ID NO: 1011 and a light chain variable region comprising an amino acid sequence that is at least 80% at least 85%, at least 90%, at least 95%, at least 98%, or 99% identical to the amino acid sequence of SEQ ID NO: 1012.

In some embodiments, the L moiety of compound 3.1 comprises an antibody that binds to GPC3. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 1013, 1014, 1015, 1016, 1017, and 1018 respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 1013, 1014, 1015, 1016, 1017, and 1018, respectively. In some embodiments, the anti-TIGIT antibody comprises a heavy chain variable region comprising an amino acid sequence that is at least 80%, at least 85%, at least 90%, at least 95%, at least 98%, or 99% identical to the amino acid sequence of SEQ ID NO: 1027 and a light chain variable region comprising an amino acid sequence that is at least 80% at least 85%, at least 90%, at least 95%, at least 98%, or 99% identical to the amino acid sequence of SEQ ID NO: 1028.

In some embodiments, the L moiety of compound 3.1 comprises an antibody that binds to TIGIT. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 1021, 1022, 1023, 1024, 1025, and 1026, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 1021, 1022, 1023, 1024, 1025, and 1026, respectively. In some embodiments, the anti-BCMA antibody comprises a heavy chain variable region comprising an amino acid sequence that is at least 80%, at least 85%, at least 90%, at least 95%, at least 98%, or 99% identical to the amino acid sequence of SEQ ID NO: 1043 and a light chain variable region comprising an amino acid sequence that is at least 80% at least 85%, at least 90%, at least 95%, at least 98%, or 99% identical to the amino acid sequence of SEQ ID NO: 1044.

In some embodiments, the L moiety of compound 3.1 comprises an antibody that binds to CD33. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 1029, 1030, 1031, 1032, 1033, and 1034, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 1029, 1030, 1031, 1032, 1033, and 1034, respectively. In some embodiments, the anti-CD33 antibody comprises a heavy chain variable region comprising an amino acid sequence that is at least 80%, at least 85%, at least 90%, at least 95%, at least 98%, or 99% identical to the amino acid sequence of SEQ ID NO: 1035 and a light chain variable region comprising an amino acid sequence that is at least 80% at least 85%, at least 90%, at least 95%, at least 98%, or 99% identical to the amino acid sequence of SEQ ID NO: 1036.

In some embodiments, the L moiety of compound 3.1 comprises an antibody that binds to BCMA. In some such cases, the antibody comprises CDR-H1, CDR-H2, CDR-H3, CDR-L1, CDR-L2, and CDR-L3 sequences comprising at least 80% sequence identity, at least 85% sequence identity, at least 90% sequence identity, or at least 95% sequence identity to the amino acid sequences of SEQ ID NOs: 1037, 1038, 1039, 1040, 1041, and 1042, respectively. In some cases, the antibody comprises CDR-H1, CDR-H2, CDR-H3, CDR-L1, CDR-L2, and CDR-L3 sequences each comprising at most one mutation relative to the amino acid sequences of SEQ ID NOs: 1037, 1038, 1039, 1040, 1041, and 1042, respectively. In some embodiments, the anti-BCMA antibody comprises a heavy chain variable region comprising an amino acid sequence that is at least 80%, at least 85%, at least 90%, at least 95%, at least 98%, or 99% identical to the amino acid sequence of SEQ ID NO: 1043 and a light chain variable region comprising an amino acid sequence that is at least 80% at least 85%, at least 90%, at least 95%, at least 98%, or 99% identical to the amino acid sequence of SEQ ID NO: 1044.

Throughout this application, unless the context indicates otherwise, references to a compound of Formula (I), (I′), (I*), (II), or any subformula thereof, includes all subgroups of Formula (I), (I′), (I*), (II), or any subformula thereof, defined herein, including all substructures, subgenera, preferences, embodiments, examples and particular compounds defined and/or described herein. References to a compound of Formula (I), (I′), (I*), (II), or any subformula thereof, include ionic forms, polymorphs, pseudopolymorphs, amorphous forms, solvates, co-crystals, chelates, isomers, tautomers, oxides (e.g., N-oxides, S-oxides), esters, prodrugs, isotopes and/or protected forms thereof. In some embodiments, references to a compound of Formula (I), (I′), (I*), (II), or any subformula thereof, include polymorphs, solvates, co-crystals, isomers, tautomers and/or oxides thereof. In some embodiments, references to a compound of Formula (I), (I′), (I*), (II), or any subformula thereof, include polymorphs, solvates, and/or co-crystals thereof. In some embodiments, references to a compound of Formula (I), (I′), (I*), (II), or any subformula thereof, include isomers, tautomers and/or oxides thereof. In some embodiments, references to a compound of Formula (I), (I′), (I*), (II), or any subformula thereof, include solvates thereof. Similarly, the term “salts” includes solvates of salts of compounds.

Any formula given herein, such as Formula (I), (I′), (I*), (II), or any subformula thereof, is intended to represent compounds having structures depicted by the structural formula as well as certain variations or forms. In particular, compounds of any formula given herein may have asymmetric centers and therefore exist in different enantiomeric or diastereomeric forms. All optical isomers and stereoisomers of the compounds of the general formula, and mixtures thereof in any ratio, are considered within the scope of the formula. Thus, any formula given herein is intended to represent a racemate, one or more enantiomeric forms, one or more diastereomeric forms, one or more atropisomeric forms, and mixtures thereof in any ratio. Where a compound of one of Tables 1, 2, or 3 is depicted with a particular stereochemical configuration, also provided herein is any alternative stereochemical configuration of the compound, as well as a mixture of stereoisomers of the compound in any ratio. For example, where a compound of Tables 1, 2, or 3 has a stereocenter that is in an “S” stereochemical configuration, also provided herein is enantiomer of the compound wherein that stereocenter is in an “R” stereochemical configuration. Likewise, when a compound of Tables 1, 2, or 3 has a stereocenter that is in an “R” configuration, also provided herein is enantiomer of the compound in an “S” stereochemical configuration. Also provided are mixtures of the compound with both the “S” and the “R” stereochemical configuration. Additionally, if a compound of Tables 1, 2, or 3 has two or more stereocenters, also provided are any enantiomer or diastereomer of the compound. For example, if a compound of Tables 1, 2, or 3 contains a first stereocenter and a second stereocenter with “R” and “R” stereochemical configurations, respectively, also provided are stereoisomers of the compound having first and second stereocenters with “S” and “S” stereochemical configurations, respectively, “S” and “R” stereochemical configurations, respectively, and “R” and “S” stereochemical configurations, respectively. If a compound of Tables 1, 2, or 3 contains a first stereocenter and a second stereocenter with “S” and “S” stereochemical configurations, respectively, also provided are stereoisomers of the compound having first and second stereocenters with “R” and “R” stereochemical configurations, respectively, “S” and “R” stereochemical configurations, respectively, and “R” and “S” stereochemical configurations, respectively. If a compound of Tables 1, 2, or 3 contains a first stereocenter and a second stereocenter with “S” and “R” stereochemical configurations, respectively, also provided are stereoisomers of the compound having first and second stereocenters with “R” and “S” stereochemical configurations, respectively, “R” and “R” stereochemical configurations, respectively, and “S” and “S” stereochemical configurations, respectively. Similarly, if a compound of Tables 1, 2, or 3 contains a first stereocenter and a second stereocenter with “R” and “S” stereochemical configurations, respectively, also provided are stereoisomers of the compound having first and second stereocenters with “S” and “R” stereochemical configurations, respectively, “R” and “R” stereochemical configurations, respectively, and “S” and “S” stereochemical configurations, respectively. Furthermore, certain structures may exist as geometric isomers (i.e., cis and trans isomers), as tautomers, or as atropisomers. Additionally, any formula given herein is intended to refer also to any one of hydrates, solvates, and amorphous and polymorphic forms of such compounds, and mixtures thereof, even if such forms are not listed explicitly. In some embodiments, the solvent is water and the solvates are hydrates.

The compounds depicted herein may be present as salts even if salts are not depicted, and it is understood that the compositions and methods provided herein embrace all salts and solvates of the compounds depicted here, as well as the non-salt and non-solvate form of the compound, as is well understood by the skilled artisan. In some embodiments, the salts of the compounds provided herein are pharmaceutically acceptable salts.

Embodiments are contemplated wherein any variation or embodiment of Q, D, Z or Z′, A, B, S*, RL, Y, W, L, Z^{a}, X^{b}, R¹, R², R³, R⁴, R⁵, R⁶, R⁷, R⁸, R⁹, R¹⁰, R^{a}, R^{b}, E, X, subscript n, subscript n, subscript q, and/or subscript p provided herein is combined with every other variation or embodiment of Q, D, Z or Z′, A, B, S*, RL, Y, W, L, Z^{a}, X^{b}, R¹, R², R³, R⁴, R⁵, R⁶, R⁷, R⁸, R⁹, R¹⁰, R^{a}, R^{b}, E, X, subscript n, subscript n, subscript q, and/or subscript p, as if each combination had been individually and specifically described.

In some embodiments, any of the compounds described herein, such as a compound of Formula (I), (I′), (I*), (II), or any subformula thereof, or a compound of any one of Tables 1, 2 or 3, or a salt of any of the foregoing, may be deuterated (e.g., a hydrogen atom is replaced by a deuterium atom). In some of these variations, the compound is deuterated at a single site. In other variations, the compound is deuterated at multiple sites. Deuterated compounds are sometimes prepared from deuterated starting materials in a manner similar to the preparation of the corresponding non-deuterated compounds. In some aspects, hydrogen atoms are be replaced with deuterium atoms using other methods known in the art.

Other embodiments will be apparent to those skilled in the art from the following detailed description.

As used herein, when any variable occurs more than one time in a chemical formula, its definition on each occurrence is independent of its definition at every other occurrence.

### Other Exemplary Ligand-Drug Conjugates

gpNMB is a type 1 transmembrane protein that has been shown to be pro-metastatic, and high expression is correlated with worse cancer patient survival. Taya et al., Steroids, 2017, 133:102. It is expressed in many cancer indications, including melanoma, squamous non-small-cell lung cancer (sqNSCLC), Head and Neck, and esophageal cancers. An antibody-drug conjugate, glembatumumab vedotin, was in melanoma and breast cancer clinical trials, but activity was limited by off target toxicities at the MTD (Ott et al., Cancer, 2019, 125:1113). Therefore, the present inventors conceived that a gpNMB-targeted antibody-drug conjugate would pair well with a payload with an improved therapeutic index.

Thus, provided herein are ligand-drug conjugates, such as of compound 3.1, wherein the L moiety is an anti-gpNMB antibody, or antigen-binding fragment thereof.

The antibody, or antigen-binding fragment thereof, in some embodiments bind to gpNMB with an affinity (e.g., EC₅₀) of less than 10 nM, 5 nM, 2 nM, 1 nM, 500 pM, 250 pM, 100 pM, 50 pM, 25 pM, 10 pM, or 1 pM. In some embodiments, the antibody, or antigen-binding fragment thereof, binds to gpNMB with an affinity of between 5-10 nM, 1-5 nM, 500 pM-1 nM, 100-250 pM, 50-100 pM, 10-50 pM, or 1-10 pM.

In some embodiments, the antibody, or antigen-binding fragment thereof, comprises the CDRs, the variable domains, and/or the heavy and light chain amino acid sequences as indicated in Table D1 and D2, below.

TABLE D1
Exemplary anti-gpNMB antibody CDR sequences.

| SEQ ID NO | Description | Sequence |
|---|---|---|
| 935 | Anti-gpNMB CDR-H1 | SFNYYWS |
| 936 | Anti-gpNMB CDR-H2 | YIYYSGSTYSNPSLKS |
| 937 | Anti-gpNMB CDR-H3 | GYNWNYFDY |
| 938 | Anti-gpNMB CDR-L1 | RASQSVDNNLV |
| 939 | Anti-gpNMB CDR-L2 | GASTRAT |
| 940 | Anti-gpNMB CDR-L3 | QQYNNWPPWT |

TABLE D2
Exemplary anti-gpNMB variable domain, light chain, and heavy
chain sequences.

| Description | Sequence (SEQ ID NO) |
|---|---|
| Anti-gpNMB variable | QVQLQESGPGLVKPSQTLSLTCTVSGGSISSFNYYWS |
| heavy (VH) | WIRHHPGKGLEWIGYIYYSGSTYSNPSLKSRVTISVDT |
| | SKNQFSLTLSSVTAADTAVYYCARGYNWNYFDYWG |
| | QGTLVTVSS (SEQ ID NO: 941) |
| Anti-gpNMB variable | EIVMTQSPATLSVSPGERATLSCRASQSVDNNLVWYQ |
| light (VL) | QKPGQAPRLLIYGASTRATGIPARFSGSGSGTEFTLTIS |
| | SLQSEDFAVYYCQQYNNWPPWTFGQGTKVEIK (SEQ |
| | ID NO: 942) |
| Anti-gpNMB heavy | QVQLQESGPGLVKPSQTLSLTCTVSGGSISSFNYYWS |
| chain | WIRHHPGKGLEWIGYIYYSGSTYSNPSLKSRVTISVDT |
| | SKNQFSLTLSSVTAADTAVYYCARGYNWNYFDYWG |
| | QGTLVTVSSASTKGPSVFPLAPSSKSTSGGTAALGCLV |
| | KDYFPEPVTVSWNSGALTSGVHTFPAVLQSSGLYSLS |
| | SVVTVPSSSLGTQTYICNVNHKPSNTKVDKKVEPKSC |
| | DKTHTCPPCPAPELLGGPSVFLFPPKPKDTLMISRTPEV |
| | TCVVVDVSHEDPEVKFNWYVDGVEVHNAKTKPREE |
| | QYNSTYRVVSVLTVLHQDWLNGKEYKCKVSNKALP |
| | APIEKTISKAKGQPREPQVYTLPPSRDELTKNQVSLTC |
| | LVKGFYPSDIAVEWESNGQPENNYKTTPPVLDSDGSF |
| | FLYSKLTVDKSRWQQGNVFSCSVMHEALHNHYTQKS |
| | LSLSPGK (SEQ ID NO: 943) |
| Anti-gpNMB light | EIVMTQSPATLSVSPGERATLSCRASQSVDNNLVWYQ |
| chain | QKPGQAPRLLIYGASTRATGIPARFSGSGSGTEFTLTIS |
| | SLQSEDFAVYYCQQYNNWPPWTFGQGTKVEIKRTVA |
| | APSVFIFPPSDEQLKSGTASVVCLLNNFYPREAKVQW |
| | KVDNALQSGNSQESVTEQDSKDSTYSLSSTLTLSKAD |
| | YEKHKVYACEVTHQGLSSPVTKSFNRGEC (SEQ ID |
| | NO: 944) |

Certain of the antibodies, or antigen-binding fragments thereof, disclosed herein comprise the 6 CDRs as follows: a CDR-H1, a CDR-H2, a CDR-H3, a CDR-L1, a CDR-L2, and a CDR-L3 comprising the amino acid sequences of SEQ ID NOs:935-940, respectively.

Some of the antibodies, or antigen-binding fragments thereof, disclosed herein comprise a VH comprising the amino acid sequence of SEQ ID NO:941 and a VL comprising the amino acid sequence of SEQ ID NO:942.

Some of the antibodies, or antigen-binding fragments thereof, disclosed herein comprise a HC comprising the amino acid sequence of SEQ ID NO:943 and a LC comprising the amino acid sequence of SEQ ID NO:944.

In other embodiments, the antibody, or antigen-binding fragment thereof, that are provided include or are derived from one or more of the CDRs, variable heavy chains, variable light chains, heavy chains, and/or light chains of the antibodies listed in Tables D1 and D2, or variants or derivatives thereof.

Certain of the antibodies, or antigen-binding fragments thereof, disclosed herein comprise the 6 CDRs as follows: a CDR-H1, a CDR-H2, a CDR-H3 from the variable heavy chain sequence comprising the amino acid sequence of SEQ ID NO:941, and a CDR-L1, a CDR-L2, and a CDR-L3 from the variable light chain sequence comprising the amino acid sequence of SEQ ID NO:942, wherein the CDRs are defined by the IMGT CDR numbering scheme.

Certain of the antibodies, or antigen-binding fragments thereof, disclosed herein comprise the 6 CDRs as follows: a CDR-H1, a CDR-H2, a CDR-H3 from the variable heavy chain sequence comprising the amino acid sequence of SEQ ID NO:941, and a CDR-L1, a CDR-L2, and a CDR-L3 from the variable light chain sequence comprising the amino acid sequence of SEQ ID NO:942, wherein the CDRs are defined by the Kabat CDR numbering scheme.

Certain of the antibodies, or antigen-binding fragments thereof, disclosed herein comprise the 6 CDRs as follows: a CDR-H1, a CDR-H2, a CDR-H3 from the variable heavy chain sequence comprising the amino acid sequence of SEQ ID NO:941, and a CDR-L1, a CDR-L2, and a CDR-L3 from the variable light chain sequence comprising the amino acid sequence of SEQ ID NO:942, wherein the CDRs are defined by the AbM CDR numbering scheme.

Certain of the antibodies, or antigen-binding fragments thereof, disclosed herein comprise the 6 CDRs as follows: a CDR-H1, a CDR-H2, a CDR-H3 from the variable heavy chain sequence comprising the amino acid sequence of SEQ ID NO:941, and a CDR-L1, a CDR-L2, and a CDR-L3 from the variable light chain sequence comprising the amino acid sequence of SEQ ID NO:942, wherein the CDRs are defined by the Chothia CDR numbering scheme.

Certain of the antibodies, or antigen-binding fragments thereof, disclosed herein comprise the 6 CDRs as follows: a CDR-H1, a CDR-H2, a CDR-H3 from the variable heavy chain sequence comprising the amino acid sequence of SEQ ID NO:941, and a CDR-L1, a CDR-L2, and a CDR-L3 from the variable light chain sequence comprising the amino acid sequence of SEQ ID NO:942, wherein the CDRs are defined by the Contact CDR numbering scheme.

Certain of the antibodies, or antigen-binding fragments thereof, disclosed herein comprise a VH comprising a CDR-H1, a CDR-H2, and a CDR-H3, wherein the CDRs of the VH collectively have at most 1, 2, 3, 4, or 5 amino acid changes relative to a corresponding CDR reference sequence, and wherein the CDR-H1 reference sequence has the amino acid sequence of SEQ ID NO:935, the CDR-H2 reference sequence has the amino acid sequence of SEQ ID NO:936, and the CDR-H3 reference sequence has the amino acid sequence of SEQ ID NO:937. In such embodiments, the amino acid changes typically are insertions, deletions and/or substitutions. In some of these embodiments, the collective number of amino acid changes are 1-3; in other embodiments, the collective number of amino acid changes are 1 or 2. In certain of the foregoing embodiments, the changes are conservative amino acid substitutions.

Certain of the antibodies, or antigen-binding fragments thereof, disclosed herein comprise a VL comprising a CDR-L1, a CDR-L2, and a CDR-L3, wherein the CDRs of the VL collectively have at most 1, 2, 3, 4, or 5 amino acid changes relative to a corresponding CDR reference sequence, and wherein the CDR-L1 reference sequence has the amino acid sequence of SEQ ID NO:938, the CDR-L2 reference sequence has the amino acid sequence of SEQ ID NO:939, and the CDR-L3 reference sequence has the amino acid sequence of SEQ ID NO:940. In such embodiments, the amino acid changes typically are insertions, deletions and/or substitutions. In some of these embodiments, the collective number of amino acid changes are 1-3; in other embodiments, the collective number of amino acid changes are 1 or 2. In certain of the foregoing embodiments, the changes are conservative amino acid substitutions.

Certain of the antibodies, or antigen-binding fragments thereof, disclosed herein comprise a VH comprising a CDR-H1, a CDR-H2, and a CDR-H3, wherein the CDRs of the VH collectively have at most 1, 2, 3, 4, or 5 amino acid changes relative to a corresponding CDR reference sequence, and wherein the CDR-H1 reference sequence has the amino acid sequence of SEQ ID NO:935, the CDR-H2 reference sequence has the amino acid sequence of SEQ ID NO:936, and the CDR-H3 reference sequence has the amino acid sequence of SEQ ID NO:937; and a VL comprising a CDR-L1, a CDR-L2, and a CDR-L3, wherein the CDRs of the VL collectively have at most 1, 2, 3, 4, or 5 amino acid changes relative to a corresponding CDR reference sequence, and wherein the CDR-L1 reference sequence has the amino acid sequence of SEQ ID NO:938, the CDR-L2 reference sequence has the amino acid sequence of SEQ ID NO:939, and the CDR-L3 reference sequence has the amino acid sequence of SEQ ID NO:940. In such embodiments, the amino acid changes typically are insertions, deletions and/or substitutions. In some of these embodiments, the collective number of amino acid changes are 1-3; in other embodiments, the collective number of amino acid changes are 1 or 2. In certain of the foregoing embodiments, the changes are conservative amino acid substitutions.

In some embodiments, the antibody, or antigen-binding fragment thereof, comprises a VH domain, wherein the VH domain sequence has at least 80%, 85%, 90%, 91%, 92%, 93%, 94%, 95%, 96%, 97%, 98%, or 99% sequence identity to the amino acid sequence of SEQ ID NO:941, provided the antibody, or antigen-binding fragment thereof, retains the ability to bind to gpNMB. In certain embodiments, such an antibody, or antigen-binding fragment thereof, contains substitutions (e.g., conservative substitutions), insertions, and/or deletions relative to the reference sequence (i.e., SEQ ID NO:941), provided that such an antibody, or antigen-binding fragment thereof, retains the ability to bind to gpNMB. In certain embodiments, 1, 2, 3, 4, 5, 6, 7, 8, 9, or 10 amino acids have been substituted, inserted and/or deleted in SEQ ID NO:941. In some embodiments, 1-5 or 1-3 amino acids have been substituted, inserted and/or deleted in the VH sequence. In certain of these embodiments, such substitutions, insertions, or deletions occur in regions outside the CDRs (i.e., in the framework regions).

In some embodiments, the antibody, or antigen-binding fragment thereof, comprises a VL domain, wherein the VL domain sequence has at least 80%, 85%, 90%, 91%, 92%, 93%, 94%, 95%, 96%, 97%, 98%, or 99% sequence identity to the amino acid sequence of SEQ ID NO:942, provided the antibody, or antigen-binding fragment thereof, retains the ability to bind to gpNMB. In certain embodiments, such an antibody, or antigen-binding fragment thereof, contains substitutions (e.g., conservative substitutions), insertions, and/or deletions relative to the reference sequence (i.e., SEQ ID NO:942), provided that such an antibody, or antigen-binding fragment thereof, retains the ability to bind to gpNMB. In certain embodiments, 1, 2, 3, 4, 5, 6, 7, 8, 9, or 10 amino acids have been substituted, inserted and/or deleted in SEQ ID NO:942. In some embodiments, 1-5 or 1-3 amino acids have been substituted, inserted and/or deleted in the VL sequence. In certain of these embodiments, such substitutions, insertions, or deletions occur in regions outside the CDRs (i.e., in the framework regions).

In some embodiments, the antibody, or antigen-binding fragment thereof, comprises: a VH domain, wherein the VH domain sequence has at least 80%, 85%, 90%, 91%, 92%, 93%, 94%, 95%, 96%, 97%, 98%, or 99% sequence identity to the amino acid sequence of SEQ ID NO:941, provided the antibody, or antigen-binding fragment thereof, retains the ability to bind to gpNMB; and a VL domain, wherein the VL domain sequence has at least 80%, 85%, 90%, 91%, 92%, 93%, 94%, 95%, 96%, 97%, 98%, or 99% sequence identity to the amino acid sequence of SEQ ID NO:942, provided the antibody, or antigen-binding fragment thereof, retains the ability to bind to gpNMB. In certain embodiments, such an antibody, or antigen-binding fragment thereof, contains substitutions (e.g., conservative substitutions), insertions, and/or deletions relative to the reference sequence (i.e., SEQ ID NO:941 for the VH domain and SEQ ID NO:942 for the VL domain), provided that such an antibody, or antigen-binding fragment thereof, retains the ability to bind to gpNMB. In certain embodiments, 1, 2, 3, 4, 5, 6, 7, 8, 9, or 10 amino acids have been substituted, inserted and/or deleted in the VH and/or the VL sequence. In some embodiments, 1-5 or 1-3 amino acids have been substituted, inserted and/or deleted in the VH and/or VL sequence. In other embodiments, 1-5 or 1-3 amino acids have been substituted, inserted and/or deleted in the VH and VL sequence collectively. In certain of these embodiments, such substitutions, insertions, or deletions occur in regions outside the CDRs (i.e., in the FRs).

In a further embodiment, an antibody, or antigen-binding fragment thereof, comprises an HC comprising the amino acid sequence of SEQ ID NO:943, and a LC comprising the amino acid sequence of SEQ ID NO:944.

The antibody, or antigen-binding fragment thereof, as described herein can be an antibody in any form. As such, the antibody, or antigen-binding fragment thereof, described in any of the above embodiments can be monoclonal, and may be multispecific, human, humanized or chimeric antibodies, single chain antibodies, Fab fragments, F(ab′) fragments, fragments produced by a Fab expression library, and gpNMB-binding fragments of any of the above. The antibodies can be of any immunoglobulin isotype (e.g., IgG, IgE, IgM, IgD, IgA and IgY), class (e.g., IgG1, IgG2, IgG3, IgG4, IgA1 and IgA2) or subclass.

In certain embodiments, an antibody, or antigen-binding fragment thereof, with the CDRs and/or variable domain sequences described herein is an antigen-binding fragment (e.g., human antigen-binding fragments) including, but are not limited to, Fab, Fab′ and F(ab′)₂, Fd, single-chain Fvs (scFv), single-chain antibodies, disulfide-linked Fvs (sdFv) and fragments comprising either a VL or VH domain.

The antibody, or antigen-binding fragment thereof, can be monospecific or part of a bispecific or trispecific antibody, or antigen-binding fragments thereof, or part of an antibody, or antigen-binding fragment thereof, of greater multi-specificity. Multispecific antibodies can be specific for different epitopes of gpNMB or may be specific for both gpNMB and a heterologous protein.

The antibodies, or antigen-binding fragments thereof, in any of the foregoing embodiments can be an antibody in any form. As such, the antibody, or antigen-binding fragment thereof, described in any of the above embodiments can be, for example, a monoclonal antibody, a multispecific antibody, a human, humanized or chimeric antibody, and gpNMB binding fragments of any of the above, such as a single chain antibody, an Fab fragment, an F(ab′) fragment, or a fragment produced by a Fab expression library. The antibodies can be of any immunoglobulin isotype (e.g., IgG, IgE, IgM, IgD, IgA and IgY), class (e.g., IgG1, IgG2, IgG3, IgG4, IgA1 and IgA2) or subclass.

In certain embodiments, a antibody, or antigen-binding fragment thereof, with the CDR and/or variable domain sequences described herein is an antigen-binding fragment (e.g., human antigen-binding fragments) and include, but are not limited to, Fab, Fab′ and F(ab′)₂, Fd, single-chain Fvs (scFv), single-chain antibodies, disulfide-linked Fvs (sdFv) and fragments comprising either a VL or VH domain. Antigen-binding fragments, including single-chain antibodies, may comprise the variable region(s) alone or in combination with the entirety or a portion of the following: hinge region CH1, CH₂, CH₃ and CL domains. Also included in the present disclosure are antigen-binding fragments comprising any combination of variable region(s) with a hinge region, CH1, CH₂, CH₃ and CL domains.

The antibody, or antigen-binding fragment thereof, can be monospecific, bispecific, trispecific or of greater multi specificity. Multispecific antibodies can be specific for different epitopes of gpNMB or may be specific for both gpNMB as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; and Kostelny et al., 1992, J. Immunol. 148:1547 1553.

In any of the embodiments described herein, one or several amino acids (e.g., 1, 2, 3 or 4) at the amino or carboxy terminus of the light and/or heavy chain, such as the C-terminal lysine of the heavy chain, may be missing or derivitized in some or all of the molecules in a composition. One specific example of such a modification, is an antibody, or antigen-binding fragment thereof, in which the carboxy terminal lysine of the heavy chain is missing (e.g., as part of a post-translational modification). Furthermore, it should be understood that any of the sequences described herein include post-translational modifications to the specified sequence during expression of the antibody, or antigen-binding fragment thereof, in cell culture (e.g., a CHO cell culture).

In other embodiments, the moiety L of compound 3.1 is not an antibody or antigen-binding fragment thereof but instead comprises a non-antibody scaffold into which one or more CDRs (e.g., 1, 2, 3, 4, 5 or 6) and/or one or more variable domains as described herein is grafted, inserted, and/or joined.

### Chimeric Antibody, or an Antigen-Binding Fragment Thereof

In certain embodiments, the antibody, or antigen-binding fragment thereof, such as the antibody, or antigen-binding fragment thereof, which is the L moiety of compound 3.1, provided herein is a chimeric antibody. In some embodiments, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Certain chimeric antibodies are described, e.g., in U.S. Pat. No. 4,816,567; and Morrison et al., (1984) Proc. Natl. Acad. Sci. USA, 81:6851-6855 (1984)). Chimeric antibodies include antigen-binding fragments thereof. Nonlimiting exemplary chimeric antibodies include chimeric antibodies comprising any of the heavy and/or light chain variable regions as described herein. Additional nonlimiting exemplary chimeric antibodies include chimeric antibodies comprising heavy chain CDR sequences or portions thereof, and/or light chain CDR sequences as provided herein.

### Humanized Antibody, or Antigen-Binding Fragment Thereof

In certain embodiments, the antibody, or antigen-binding fragment thereof, is a humanized antibody, or antigen-binding fragment thereof, that binds gpNMB. Typically, a non-human antibody is humanized to reduce immunogenicity to humans, while retaining the specificity and affinity of the parental non-human antibody. A humanized antibody is a genetically engineered antibody in which the CDRs or portions thereof from a non-human “donor” antibody are grafted into human “acceptor” antibody sequences (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557).

The acceptor antibody sequences can be, for example, a mature human antibody sequence, a composite of such sequences, a consensus sequence of human antibody sequences, or a germline region sequence. Human acceptor sequences can be selected for a high degree of sequence identity in the variable region frameworks with donor sequences to match canonical forms between acceptor and donor CDRs among other criteria. Thus, a humanized antibody is an antibody having CDRs entirely or substantially from a donor antibody and variable region framework sequences and constant regions, if present, entirely or substantially from human antibody sequences. Similarly, a humanized heavy chain typically has all three CDRs entirely or substantially from a donor antibody heavy chain, and a heavy chain variable region framework sequence and heavy chain constant region, if present, substantially from human heavy chain variable region framework and constant region sequences. Likewise, a humanized light chain usually has all three CDRs entirely or substantially from a donor antibody light chain, and a light chain variable region framework sequence and light chain constant region, if present, substantially from human light chain variable region framework and constant region sequences. A CDR in a humanized antibody is substantially from a corresponding CDR in a non-human antibody when at least 80%, 85%, 90%, 95% or 100% of corresponding residues (such as defined by Kabat) are identical between the respective CDRs. The variable region framework sequences of an antibody chain or the constant region of an antibody chain are substantially from a human variable region framework sequence or human constant region respectively when at least 80%, 85%, 90%, 95% or 100% of corresponding residues, such as defined by Kabat, are identical.

For example, when an amino acid differs between a murine variable region framework residue and a selected human variable region framework residue, the human framework amino acid can be substituted by the equivalent framework amino acid from the mouse antibody when it is reasonably expected that the amino acid:
(1) noncovalently binds antigen directly,
(2) is adjacent to a CDR region,
(3) otherwise interacts with a CDR region (e.g., is within about 6 Å of such a region);
(4) mediates interaction between the heavy and light chains, or
(5) is the result of somatic mutation in the mouse chain.
(6) is a site of glycosylation.

Framework residues from classes (1)-(3) are sometimes alternately referred to as canonical and vernier residues. Canonical residues refer to framework residues defining the canonical class of the donor CDR loops determining the conformation of a CDR loop (Chothia and Lesk, J. Mol. Biol. 196, 901-917 (1987), Thornton & Martin, J. Mol. Biol., 263, 800-815, 1996). Vernier residues refer to a layer of framework residues that support antigen-binding loop conformations and play a role in fine-tuning the fit of an antibody to antigen (Foote & Winter, 1992, J Mol Bio. 224, 487-499).

Humanized antibodies and methods of making them are reviewed, e.g., in Almagro and Fransson, (2008) Front. Biosci. 13: 1619-1633, and are further described, e.g., in Riechmann et al., (1988) Nature 332:323-329; Queen et al., (1989) Proc. Natl Acad. Sci. USA 86: 10029-10033; U.S. Pat. Nos. 5,821,337, 7,527,791, 6,982,321, and 7,087,409; Kashmiri et al., (2005) Methods 36:25-34 (describing specificity determining region (SDR) grafting); Padlan, (1991) Mol. Immunol. 28:489-498 (describing “resurfacing”); Dall'Acqua et al., (2005) Methods 36:43-60 (describing “FR shuffling”); and Osbourn et al., (2005) Methods 36:61-68 and Klimka et al., (2000) Br. J. Cancer, 83:252-260 (describing the “guided selection” approach to FR shuffling).

Human framework regions that may be used for humanization include but are not limited to: framework regions selected using the “best-fit” method (see, e.g., Sims et al. (1993) J. Immunol. 151:2296); framework regions derived from the consensus sequence of human antibodies of a particular subgroup of light or heavy chain variable regions (see, e.g., Carter et al. (1992) Proc. Natl. Acad. Sci. USA, 89:4285; and Presta et al. (1993) J. Immunol, 151:2623); human mature (somatically mutated) framework regions or human germline framework regions (see, e.g., Almagro and Fransson, (2008) Front. Biosci. 13:1619-1633); and framework regions derived from screening FR libraries (see, e.g., Baca et al., (1997) J. Biol. Chem. 272: 10678-10684 and Rosok et al., (1996) J. Biol. Chem. 271:22611-22618).

Non-limiting exemplary chimeric antibodies include chimeric antibodies comprising or derived from any of the CDR and/or heavy chain variable region and/or light chain variable regions as disclosed herein.

Although humanized antibodies often incorporate all six HVRs (e.g., CDRs, preferably as defined by Kabat) from a mouse antibody, they can also be made with less than all HVRs or CDRs (e.g., at least 3, 4, or 5) HVRs or CDRs from a mouse antibody (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; and Tamura et al, Journal of Immunology, 164:1432-1441, 2000).

Certain amino acids from the human variable region framework residues can be selected for substitution based on their possible influence on HVR (e.g., CDR) conformation and/or binding to antigen. Investigation of such possible influences is by modeling, examination of the characteristics of the amino acids at particular locations, or empirical observation of the effects of substitution or mutagenesis of particular amino acids.

### Exemplary Antibody Constant Regions

In some embodiments, the heavy and light chain variable regions of antibodies, or antigen-binding fragments thereof, described herein can be linked to at least a portion of a human constant region. In some embodiments, the human heavy chain constant region is of an isotype selected from IgA, IgG, and IgD. In some embodiments, the human light chain constant region is of an isotype selected from κ and λ. In some embodiments, an antibody described herein comprises a human IgG constant region. In some embodiments, an antibody described herein comprises a human IgG4 heavy chain constant region. In some of these embodiments, an antibody described herein comprises an S241P mutation in the human IgG4 constant region. In some embodiments, an antibody described herein comprises a human IgG4 constant region and a human κ light chain.

Throughout the present specification and claims unless explicitly stated or known to one skilled in the art, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

Human constant regions show allotypic variation and isoallotypic variation between different individuals, that is, the constant regions can differ in different individuals at one or more polymorphic positions. Isoallotypes differ from allotypes in that sera recognizing an isoallotype binds to a non-polymorphic region of a one or more other isotypes. Reference to a human constant region includes a constant region with any natural allotype or any permutation of residues occupying polymorphic positions in natural allotypes. Also, up to 1, 2, 5, or 10 mutations may be present relative to a natural human constant region, such as those indicated above to reduce Fcγ receptor binding or increase binding to FcRn.

In some embodiments, one or several amino acids at the amino or carboxy terminus of the light and/or heavy chain, such as the C-terminal lysine of the heavy chain, may be missing or derivatized in a proportion or all of the molecules.

The choice of constant region depends, in part, whether antibody-dependent cell-mediated cytotoxicity, antibody dependent cellular phagocytosis and/or complement dependent cytotoxicity are desired. For example, human isotopes IgG1 and IgG3 have strong complement-dependent cytotoxicity, human isotype IgG2 weak complement-dependent cytotoxicity and human IgG4 lacks complement-dependent cytotoxicity. Human IgG1 and IgG3 also induce stronger cell-mediated effector functions than human IgG2 and IgG4. Light chain constant regions can be lambda or kappa.

Furthermore, as described in greater detail below, substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004).

### Variant Antibody or Antigen-Binding Fragments Thereof

The antibodies, or antigen-binding fragments thereof, provided herein also include amino acid sequence variants of the antibody, or antigen-binding fragment thereof, provided herein such as those described in Table D1 and D2. As an example, variants with improved binding affinity and/or other biological properties of the antibody can be prepared. Amino acid sequence variants of an antigen binding protein can be prepared by introducing appropriate modifications into the nucleotide sequence encoding the antigen binding protein, or by peptide synthesis. Such modifications include, for example, deletions from, and/or insertions into and/or substitutions of residues within the amino acid sequences of the antigen binding protein. Any combination of deletion, insertion, and substitution can be made to arrive at the final construct, provided that the final construct possesses the desired characteristics, e.g., antigen-binding.

Substitution, insertion, and deletion variants. In some embodiments, an antibody, or antigen-binding fragment thereof, is a variant in that it has one or more amino acid substitutions, deletions and/or insertions relative to an antibody, or antigen-binding fragment thereof, as described herein (e.g., an antibody, or antigen-binding fragment thereof, having the amino acid sequences as described in Tables D1 and D2. In certain such embodiments, the variant has one or more amino acid substitutions. In further such embodiments, the substitutions are conservative amino acid substitutions.

An amino acid substitution can include but are not limited to the replacement of one amino acid in a polypeptide with another amino acid. Conservative amino acid substitutions can encompass non-naturally occurring amino acid residues, which are typically incorporated by chemical peptide synthesis rather than by synthesis in biological systems. Naturally occurring residues can be divided into classes based on common side chain properties.
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Sites of interest for substitutional mutagenesis include the CDRs and framework regions. Conservative substitutions are shown in Table D3, below, under the heading of “Preferred Substitutions.” More substantial changes are provided in Table D3 under the heading of “Exemplary Substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE D3
Conservative and Exemplary Amino Acid Substitutions.

| | | Preferred |
|---|---|---|
| Original Residue | Exemplary Substitutions | Substitutions |
| Ala | Val; Leu; Ile | Val |
| Arg | Lys; Gln; Asn | Lys |
| Asn | Gln; His; Asp; Lys; Arg | Gln |
| Asp | Glu; Asn | Glu |
| Cys | Ser; Ala | Ser |
| Gln | Asn; Glu | Asn |
| Glu | Asp; Gln | Asp |
| Gly | Pro; Ala | Ala |
| His | Asn; Gln; Lys; Arg | Arg |
| Ile | Leu; Val; Met; Ala; Phe; Norleucine | Leu |
| Leu | Norleucine; Ile; Val; Met; Ala; Phe | Ile |
| Lys | Arg; Gln; Asn | Arg |
| Met | Leu; Phe; Ile | Leu |
| Phe | Trp; Leu; Val; Ile; Ala; Tyr | Leu |
| Pro | Ala | Ala |
| Ser | Thr; Ala; Cys | Thr |
| Thr | Val; Ser | Ser |
| Trp | Tyr; Phe | Tyr |
| Tyr | Trp; Phe; Thr; Ser | Phe |
| Val | Ile; Leu; Met; Phe; Ala; Norleucine | Leu |

Non-conservative substitutions involve exchanging a member of one of these classes for another class.

In altering the amino acid sequence of the antigen binding protein (e.g., anti-gpNMB antibody or antigen-binding fragments thereof), in some embodiments the hydropathic index of amino acids can be considered. Each amino acid has been assigned a hydropathic index on the basis of its hydrophobicity and charge characteristics as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., 1982, J. Mol. Biol., 157:105-131. It is known that certain amino acids can be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those which are within ±1 are included, and in certain embodiments, those within ±0.5 are included.

It is also understood in the art that the substitution of like amino acids can be made effectively on the basis of hydrophilicity, particularly where the biologically functional protein or peptide (e.g., antibody) thus created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0±1); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included. One can also identify epitopes from primary amino acid sequences on the basis of hydrophilicity. These regions are also referred to as “epitopic core regions.”

Alterations (e.g., substitutions) can be made in CDRs, e.g., to improve antibody affinity. Such alterations can be made in CDR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves hypervariable region (HVR)-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions can occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

Variants with Modified Fc Region

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In certain embodiments, an antibody variant is prepared that has improved or diminished binding to FcRs are described. (See, e.g., U.S. Pat. No. 6,737,056; WO 2004/056312, and Shields et al., J. Biol. Chem. 9(2): 6591-6604 (2001).) In some embodiments, an antibody variant comprises an Fc region with one or more amino acid substitutions which improve ADCC, e.g., substitutions at positions 298, 333, and/or 334 of the Fe region (EU numbering of residues). For instance, a systemic substitution of solvent-exposed amino acids of human IgG1 Fc region has generated IgG variants with altered FcγR binding affinities (Shields et al., 2001, J. Biol. Chem. 276:6591-604). When compared to parental IgG1, a subset of these variants involving substitutions at Thr256/Ser298, Ser298/Glu333, Ser298/Lys334, or Ser298/Glu333/Lys334 to Ala demonstrate increased in both binding affinity toward FcγR and ADCC activity (Shields et al., 2001, J. Biol. Chem. 276:6591-604; Okazaki et al., 2004, J. Mol. Biol. 336:1239-49).

In some embodiments, alterations are made in the Fc region to alter (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000). For instance, complement fixation activity of antibodies (both C1q binding and CDC activity) can be improved by substitutions at Lys326 and Glu333 (Idusogie et al., 2001, J. Immunol. 166:2571-2575). The same substitutions on a human IgG2 backbone can convert an antibody isotype that binds poorly to C1q and is severely deficient in complement activation activity to one that can both bind C1q and mediate CDC (Idusogie et al., 2001, J. Immunol. 166:2571-75). Several other methods have also been applied to improve complement fixation activity of antibodies. For example, the grafting of an 18-amino acid carboxyl-terminal tail piece of IgM to the carboxyl-termini of IgG greatly enhances their CDC activity. This is observed even with IgG4, which normally has no detectable CDC activity (Smith et al., 1995, J. Immunol. 154:2226-36). Also, substituting Ser444 located close to the carboxy-terminal of IgG1 heavy chain with Cys induced tail-to-tail dimerization of IgG1 with a 200-fold increase of CDC activity over monomeric IgG1 (Shopes et al., 1992, J. Immunol. 148:2918-22). In addition, a bispecific diabody construct with specificity for C1q also confers CDC activity (Kontermann et al., 1997, Nat. Biotech. 15:629-31).

Complement activity can be reduced by mutating at least one of the amino acid residues 318, 320, and 322 of the heavy chain to a residue having a different side chain, such as Ala. Other alkyl-substituted non-ionic residues, such as Gly, Ile, Leu, or Val, or such aromatic non-polar residues as Phe, Tyr, Trp and Pro in place of any one of the three residues also reduce or abolish C1q binding. Ser, Thr, Cys, and Met can be used at residues 320 and 322, but not 318, to reduce or abolish C1q binding activity. Replacement of the 318 (Glu) residue by a polar residue may modify but not abolish C1q binding activity. Replacing residue 297 (Asn) with Ala results in removal of lytic activity but only slightly reduces (about three fold weaker) affinity for C1q. This alteration destroys the glycosylation site and the presence of carbohydrate that is required for complement activation. Any other substitution at this site also destroys the glycosylation site. The following mutations and any combination thereof also reduce C1q binding: D270A, K322A, P329A, and P311S (see WO 06/036291).

The half-life of an antibody as provided herein can be increased or decreased to modify its therapeutic activities. FcRn is a receptor that is structurally similar to MHC Class I antigen that non-covalently associates with β2-microglobulin. FcRn regulates the catabolism of IgGs and their transcytosis across tissues (Ghetie and Ward, 2000, Annu. Rev. Immunol. 18:739-766; Ghetie and Ward, 2002, Immunol. Res. 25:97-113). The IgG-FcRn interaction takes place at pH 6.0 (pH of intracellular vesicles) but not at pH 7.4 (pH of blood); this interaction enables IgGs to be recycled back to the circulation (Ghetie and Ward, 2000, Ann. Rev. Immunol. 18:739-766; Ghetie and Ward, 2002, Immunol. Res. 25:97-113). The region on human IgG₁ involved in FcRn binding has been mapped (Shields et al., 2001, J. Biol. Chem. 276:6591-604). Alanine substitutions at positions Pro238, Thr256, Thr307, Gln311, Asp312, Glu380, Glu382, or Asn434 of human IgG1 enhance FcRn binding (Shields et al., 2001, J. Biol. Chem. 276:6591-604). IgG1 molecules harboring these substitutions have longer serum half-lives. Consequently, these modified IgG1 molecules may be able to carry out their effector functions, and hence exert their therapeutic efficacies, over a longer period of time compared to unmodified IgG₁. Other exemplary substitutions for increasing binding to FcRn include a Gln at position 250 and/or a Leu at position 428. Other studies have shown that binding of the Fc region to FcRn can be improved by introducing one or more substitutions at one or more the following Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (see, e.g., U.S. Pat. Nos. 7,371,826; and 7,361,740).

Antibody Variants with Modified Glycosylation

In certain embodiments, an antibody, or antigen-binding fragment thereof, as provided herein includes one or more modifications so as to increase or decrease the extent to which the antibody is glycosylated. Addition or deletion of glycosylation sites to an antibody can be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fe region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure.

Engineering of this glycoform on IgG can significantly improve IgG-mediated ADCC. Addition of bisecting N-acetylglucosamine modifications (Umana et al., 1999, Nat. Biotechnol. 17:176-180; Davies et al., 2001, Biotech. Bioeng. 74:288-94) to this glycoform or removal of fucose (Shields et al., 2002, J. Biol. Chem. 277:26733-40; Shinkawa et al., 2003, J. Biol. Chem. 278:6591-604; Niwa et al., 2004, Cancer Res. 64:2127-33) from this glycoform are two examples of IgG Fc engineering that improves the binding between IgG Fc and FcγR, thereby enhancing Ig-mediated ADCC activity. Antibodies including such substitutions or engineering are included in some of the embodiments provided herein.

In certain embodiments, antibodies are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Other antibodies are further provided which contain bisected oligosaccharides, e.g., in which a biantennary oligosaccharide attached to the Fc region of the antibody is bisected by GlcNAc. Such antibodies may have reduced fucosylation and/or improved ADCC function. Examples of such antibodies are described, e.g., in WO 2003/011878 (Jean-Mairet et al.); U.S. Pat. No. 6,602,684 (Umana et al.); and US 2005/0123546 (Umana et al.). Antibodies with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### Cysteine Engineered Antibody Variants

In some embodiments, an antibody variant as provided herein includes a substitution of the native amino acid to a cysteine residue at amino acid position 234, 235, 237, 239, 267, 298, 299, 326, 330, or 332, preferably an S239C mutation (substitutions of the constant regions are according to the EU index) in a human IgG1 isotype. The presence of an additional cysteine residue allows interchain disulfide bond formation. Such interchain disulfide bond formation can cause steric hindrance, thereby reducing the affinity of the Fc region-FcγR binding interaction. The cysteine residue(s) introduced in or in proximity to the Fc region of an IgG constant region can also serve as sites for conjugation to therapeutic agents (e.g., coupling cytotoxic drugs using thiol specific reagents such as maleimide derivatives of drugs). The presence of a therapeutic agent causes steric hindrance, thereby further reducing the affinity of the Fc region-FcγR binding interaction. Other substitutions at any of positions 234, 235, 236 and/or 237 reduce affinity for Fey receptors, particularly FcγRI receptor (see, e.g., U.S. Pat. Nos. 6,624,821, 5,624,821.)

In other cysteine engineered antibody variants, one or more reactive thiol groups are positioned at accessible sites of the antibody and can be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and 5400 (EU numbering) of the heavy chain Fe region. Generating of cysteine engineered antibodies are described, e.g., in U.S. Pat. No. 7,521,541.

### Competing Antibody, or Antigen-Binding Fragments Thereof

The antibodies, or antigen-binding fragments thereof, provided herein include those that compete with one of the exemplified antibodies, or antigen-binding fragment thereof, for specific binding, such as to gpNMB, such as human gpNMB. In some of these embodiments, the test and reference antibody, or antigen-binding fragment thereof, cross-compete with one another. Such an antibody, or antigen-binding fragments thereof, may bind to the same epitope as one of the antibodies, or antigen-binding fragments thereof, described herein, or to an overlapping epitope. Antibodies, or antigen-binding fragments thereof, are expected to show similar functional properties (e.g., one or more of the activities described above). The exemplified antibody, or antigen-binding fragment thereof, include those described above, including those with: 1) the heavy and/or light chains, 2) VHs and/or VLs, and/or 3) that comprise one or more of the CDRs included in Tables D1 and D2.

Thus, in some embodiments, the antibody, or antigen-binding fragment thereof, that are provided include those that compete with an antibody having: (a) all 6 of the CDRs listed for the antibody described in Table D1; (b) a VH and a VL listed for the antibody described in Table D2; or (c) the light chain and heavy chain as specified for the antibody described in Table D2.

In some embodiments, competition or cross-competition is determined by surface plasmon resonance analysis (e.g., BIACORE®) (see, e.g., Abdiche, et al., 2009, Anal. Biochem. 386:172-180; Abdiche, et al., 2012, J. Immunol Methods 382:101-116; and Abdiche, et al., 2014 PLoS One 9:e92451.

### Ligand-Drug Conjugate Mixtures and Compositions

The present invention provides Ligand-Drug Conjugate mixtures and pharmaceutical compositions comprising any of the Ligand-Drug Conjugates described herein. The mixtures and pharmaceutical compositions comprise a plurality of conjugates. In some embodiments, each of the conjugates in the mixture or composition is identical or substantially identical, however, the distribution of drug-linkers on the ligands in the mixture or compositions may vary as well as the drug loading. For example, the conjugation technology used to conjugate drug-linkers to antibodies as the targeting agent in some embodiments results in a composition or mixture that is heterogeneous with respect to the distribution of Drug-Linker compounds on the antibody (Ligand Unit) within the mixture and/or composition. In some of those embodiments, the loading of Drug-Linker compounds on each of the antibody molecules in a mixture or composition of such molecules is an integer that ranges from 1 to 16.

In those embodiments, when referring to the composition as a whole, the loading of drug-linkers is a number ranging from 1 to about 16. Within the composition or mixture, there sometimes is a small percentage of unconjugated antibodies. The average number of drug-linkers per Ligand Unit in the mixture or composition (i.e., average drug-load) is an important attribute as it determines the maximum amount of drug that is delivered to the target cell. Typically, the average drug load is 1, 2 or about 2, 3 or about 3, 4 or about 4, 5 or about 5, 6 or about 6, 7 or about 7, 8 or about 8, 9 or about 9, 10 or about 10, 11 or about 11, 12 or about 12, 13 or about 13, 14 or about 14, 15 or about 15, 16 or about 16. In some embodiments, the average drug loading is from about 1 to about 16, from about 2 to about 12, from about 3 to about 8, or about 4. In some embodiments, the average drug loading is from about 3 to about 5, from about 3.5 to about 4.5, from about 3.7 to about 4.3, from about 3.9 to about 4.3, or about 4.

In some embodiments, the mixtures and pharmaceutical compositions comprise a plurality (i.e., population) of conjugates, however, the conjugates are identical or substantially identical and are substantially homogenous with respect to the distribution of drug-linkers on the ligand molecules within the mixture and/or composition and with respect to loading of drug-linkers on the ligand molecules within the mixture and/or composition. In some such embodiments, the loading of drug-linkers on an antibody Ligand Unit is 2 or 4. Within the composition or mixture, there may also be a small percentage of unconjugated antibodies. The average drug load in such embodiments is about 2 or about 4. Typically, such compositions and mixtures result from the use of site-specific conjugation techniques and conjugation is due to an introduced cysteine residue.

The average number of Drug Units or Drug-Linker compounds per Ligand Unit in a preparation from a conjugation reaction may be characterized by conventional means such as mass spectrometry, ELISA assay, HPLC (e.g., HIC). In those instances, the quantitative distribution of Ligand-Drug Conjugates in terms of subscript p may also be determined. In other instances, separation, purification, and characterization of homogeneous Ligand-Drug Conjugates may be achieved by conventional means such as reverse phase HPLC or electrophoresis.

In some embodiments, the compositions are pharmaceutical compositions comprising the Ligand-Drug Conjugates described herein and a pharmaceutically acceptable carrier. In some of those embodiments, the pharmaceutical composition is in liquid form. In some embodiments, the pharmaceutical composition is a solid. In other of those embodiments, the pharmaceutical composition is a lyophilized powder.

The compositions, including pharmaceutical compositions, are provided in purified form. As used herein, “purified” means that when isolated, the isolate contains at least 95%, and in other embodiments at least 98%, of Conjugate by weight of the isolate.

### Methods of Use

### Treatment of Cancer

The Ligand-Drug Conjugates are useful for inhibiting the multiplication of a tumor cell or cancer cell, causing apoptosis in a tumor or cancer cell, or for treating a cancer in a patient. The Ligand-Drug Conjugates are used accordingly in a variety of settings for the treatment of cancers. The Ligand-Drug Conjugates are intended to deliver a drug to a tumor cell or cancer cell. Without being bound by theory, in one embodiment, the Ligand Unit of a Ligand-Drug Conjugate binds to or associates with a cancer-cell or a tumor-cell-associated antigen, and the Ligand-Drug Conjugate is taken up (internalized) inside the tumor cell or cancer cell through receptor-mediated endocytosis or other internalization mechanism. In some embodiments, the antigen is attached to a tumor cell or cancer cell or is an extracellular matrix protein associated with the tumor cell or cancer cell. Once inside the cell, via activation of the Activation Unit, the drug is released within the cell. In an alternative embodiment, the free drug is released from the Ligand-Drug Conjugate outside the tumor cell or cancer cell, and the free drug subsequently penetrates the cell.

In one embodiment, the Ligand Unit binds to the tumor cell or cancer cell.

In another embodiment, the Ligand Unit binds to a tumor cell or cancer cell antigen which is on the surface of the tumor cell or cancer cell.

In another embodiment, the Ligand Unit binds to a tumor cell or cancer cell antigen that is an extracellular matrix protein associated with the tumor cell or cancer cell.

The specificity of the Ligand Unit for a particular tumor cell or cancer cell is an important consideration for determining the tumors or cancers that are most effectively treated. For example, Ligand-Drug Conjugates that target a cancer cell antigen present on hematopoietic cancers are useful treating hematologic malignancies (e.g., anti-CD30, anti-CD70, anti-CD19, anti-CD33 binding Ligand Unit (e.g., antibody) are useful for treating hematologic malignancies). Ligand-Drug Conjugates that target a cancer cell antigen present on solid tumors in some embodiments are useful treating such solid tumors.

Cancers that are intended to be treated with a Drug-Linker Conjugate include, but are not limited to, hematopoietic cancers such as, for example, lymphomas (Hodgkin Lymphoma and Non-Hodgkin Lymphomas) and leukemias and solid tumors. Examples of hematopoietic cancers include, follicular lymphoma, anaplastic large cell lymphoma, mantle cell lymphoma, acute myeloblastic leukemia, chronic myelocytic leukemia, chronic lymphocytic leukemia, diffuse large B cell lymphoma, and multiple myeloma. Examples of solid tumors include fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, neuroblastoma, and retinoblastoma.

In some embodiments, the treated cancer is any one of the above-listed lymphomas and leukemias.

### Multi-Modality Therapy for Cancer

Cancers, including, but not limited to, a tumor, metastasis, or other disease or disorder characterized by uncontrolled cell growth are intended to be treated or inhibited by administration of an effective amount of a Ligand-Drug Conjugate.

In one group of embodiments, methods for treating cancer are provided, including administering to a patient in need thereof an effective amount of a Ligand-Drug Conjugate and a chemotherapeutic agent. In one embodiment the chemotherapeutic agent is one in which treatment of the cancer has not been found to be refractory to that agent. In another embodiment, the chemotherapeutic agent one in which the treatment of cancer has been found to be refractory to that agent.

In another group of embodiments, the Ligand-Drug Conjugates is administered to a patient that has also undergone surgery as treatment for the cancer. In such embodiments a chemotherapeutic agent is typically administered over a series of sessions, or one or a combination of the chemotherapeutic agents, such a standard of care chemotherapeutic agent(s), is administered.

In either group of embodiments, the patient also receives an additional treatment, such as radiation therapy. In a specific embodiment, the Ligand-Drug Conjugate is administered concurrently with the chemotherapeutic agent or with radiation therapy. In another specific embodiment, the chemotherapeutic agent or radiation therapy is administered prior or subsequent to administration of a Ligand-Drug Conjugate.

Additionally, methods of treatment of cancer with a Ligand-Drug Conjugate are provided as an alternative to chemotherapy or radiation therapy where the chemotherapy or the radiation therapy has proven or can prove too toxic, e.g., results in unacceptable or unbearable side effects, for the subject being treated. The patient being treated is optionally treated with another cancer treatment such as surgery, radiation therapy or chemotherapy, depending on which treatment is found to be acceptable or bearable.

### Treatment of Autoimmune Diseases

The Ligand-Drug Conjugates are intended to be useful for killing or inhibiting the unwanted replication of cells that produce an autoimmune disease or for treating an autoimmune disease.

The Ligand-Drug Conjugates are used accordingly in a variety of settings for the treatment of an autoimmune disease in a patient. The Ligand-Drug Conjugates are typically used to deliver a Drug Unit to a target cell. Without being bound by theory, in one embodiment, the Ligand-Drug Conjugate associates with an antigen on the surface of a pro-inflammatory or inappropriately stimulated immune cell, and the Ligand-Drug Conjugate is then taken up inside the targeted cell through receptor-mediated endocytosis. Once inside the cell, the Linker Unit is cleaved, resulting in release of the Drug Unit as free drug. The free drug is then able to migrate within the cytosol and induce a cytotoxic or cytostatic activity. In an alternative embodiment, the Drug Unit is cleaved from the Ligand-Drug Conjugate outside the target cell, and the free drug resulting from that release subsequently penetrates the cell.

In one embodiment, the Ligand Unit binds to an autoimmune antigen. In one such embodiment, the antigen is on the surface of a cell involved in an autoimmune condition.

In one embodiment, the Ligand Unit binds to activated lymphocytes that are associated with the autoimmune disease state.

In a further embodiment, the Ligand-Drug Conjugate kills or inhibits the multiplication of cells that produce an autoimmune antibody associated with a particular autoimmune disease.

Particular types of autoimmune diseases intended to be treated with the Ligand-Drug Conjugates include, but are not limited to, Th2 lymphocyte related disorders (e.g., atopic dermatitis, atopic asthma, rhinoconjunctivitis, allergic rhinitis, Omenn's syndrome, systemic sclerosis, and graft versus host disease); Th1 lymphocyte-related disorders (e.g., rheumatoid arthritis, multiple sclerosis, psoriasis, Sjorgren's syndrome, Hashimoto's thyroiditis, Grave's disease, primary biliary cirrhosis, Wegener's granulomatosis, and tuberculosis); and activated B lymphocyte-related disorders (e.g., systemic lupus erythematosus, Goodpasture's syndrome, rheumatoid arthritis, and type I diabetes).

### Multi-Drug Therapy of Autoimmune Diseases

Methods for treating an autoimmune disease are also disclosed including administering to a patient in need thereof an effective amount of a Ligand-Drug Conjugate and another therapeutic agent known for the treatment of an autoimmune disease. For any of the methods of treatment described herein, the Ligand-Drug Conjugate compound may be administered to a subject who is unable to tolerate another Ligand-Drug Conjugate compound in therapeutically effective doses. In some embodiments, the other Ligand-Drug Conjugate compound comprises monomethyl auristain E (MMAE) or monomethyl auristatin F (MMAF). In some embodiments, the patient has greater tolerance for a Ligand-Drug Conjugate compound provided herein as compared to another Ligand-Drug Conjugate compound (e.g., a Ligand-Drug Conjugate compound comprising MMAE or MMAF). Tolerability may be determined using known methods or any of the methods described herein.

### Compositions and Methods of Administration

The present invention provides pharmaceutical compositions comprising the Ligand-Drug Conjugates described herein and at least one pharmaceutically acceptable carrier. The pharmaceutical composition is in any form that allows the compound to be administered to a patient for treatment of a disorder associated with expression of the antigen to which the Ligand unit binds. For example, the conjugates are in the form of a liquid or solid. The preferred route of administration is parenteral. Parenteral administration includes subcutaneous injections, intravenous, intramuscular, intrasternal injection or infusion techniques. In one embodiment, the pharmaceutical compositions is administered parenterally. In one embodiment, the conjugates are administered intravenously. Administration is by any convenient route, for example by infusion or bolus injection.

Pharmaceutical compositions are formulated to allow a Ligand-Drug Conjugate to be bioavailable upon administration of the composition to a patient. Compositions sometimes take the form of one or more dosage units.

Materials used in preparing the pharmaceutical compositions are preferably non-toxic in the amounts used. It will be evident to those of ordinary skill in the art that the optimal dosage of the active ingredient(s) in the pharmaceutical composition will depend on a variety of factors. Relevant factors include, without limitation, the type of animal (e.g., human), the particular form of the compound, the manner of administration, and the composition employed.

The composition in some embodiments is in the form of a liquid. The liquid in some of those embodiments is useful for delivery by injection. In some embodiments a composition for administration by injection, in addition to the Ligand-Drug Conjugate, contains one or more excipients selected from the group consisting of a surfactant, preservative, wetting agent, dispersing agent, suspending agent, buffer, stabilizer and isotonic agent.

The liquid compositions, whether they are solutions, suspensions or other like form, in some embodiments include one or more of the following: sterile diluents such as water for injection, saline solution, preferably physiological saline, Ringer's solution, isotonic sodium chloride, fixed oils such as synthetic mono or digylcerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as amino acids, acetates, citrates or phosphates; detergents, such as nonionic surfactants, polyols; and agents for the adjustment of tonicity such as sodium chloride or dextrose. A parenteral composition is sometimes enclosed in ampoule, a disposable syringe or a multiple-dose vial made of glass, plastic or other material. Physiological saline is an exemplary adjuvant. An injectable composition is preferably sterile.

The amount of the conjugate that is effective in the treatment of a particular disorder or condition will depend on the nature of the disorder or condition, which in some embodiments is determined by standard clinical techniques. In addition, in vitro or in vivo assays are optionally employed to help identify optimal dosage ranges. The precise dose to be employed in the compositions will also depend on the route of administration, and the seriousness of the disease or disorder, and should be decided according to the judgment of the practitioner and each patient's circumstances.

The compositions comprise an effective amount of a Ligand-Drug Conjugate such that a suitable dosage amount will be obtained. Typically, that amount is at least about 0.01% of a compound by weight of the composition.

For intravenous administration, the pharmaceutical composition typically comprises from about 0.01 to about 100 mg of a Ligand-Drug Conjugate per kg of the animal's body weight. In one embodiment, the composition can include from about 1 to about 100 mg of a Ligand-Drug Conjugate per kg of the animal's body weight. In another aspect, the amount administered will be in the range from about 0.1 to about 25 mg/kg of body weight of a compound. In some aspects, depending on the drug used, the dosage is even lower, for example, 1.0 μg/kg to 5.0 mg/kg, 4.0 mg/kg, 3.0 mg/kg, 2.0 mg/kg or 1.0 mg/kg, or 1.0 μg/kg to 500.0 μg/kg of the subject's body weight.

Generally, the dosage of a conjugate administered to a patient is typically about 0.01 mg/kg to about 100 mg/kg of the subject's body weight or from 1.0 μg/kg to 5.0 mg/kg of the subject's body weight. In some embodiments, the dosage administered to a patient is between about 0.01 mg/kg to about 15 mg/kg of the subject's body weight. In some embodiments, the dosage administered to a patient is between about 0.1 mg/kg and about 15 mg/kg of the subject's body weight. In some embodiments, the dosage administered to a patient is between about 0.1 mg/kg and about 20 mg/kg of the subject's body weight. In some embodiments, the dosage administered is between about 0.1 mg/kg to about 5 mg/kg or about 0.1 mg/kg to about 10 mg/kg of the subject's body weight. In some embodiments, the dosage administered is between about 1 mg/kg to about 15 mg/kg of the subject's body weight. In some embodiments, the dosage administered is between about 1 mg/kg to about 10 mg/kg of the subject's body weight. In some embodiments, the dosage administered is between about 0.1 to 4 mg/kg, even more preferably 0.1 to 3.2 mg/kg, or even more preferably 0.1 to 2.7 mg/kg of the subject's body weight over a treatment cycle.

The term “carrier” refers to a diluent, adjuvant or excipient, with which a compound is administered. Such pharmaceutical carriers in some embodiments is a liquid, such as water and oils, including those of petroleum, animal, vegetable or synthetic origin, such as peanut oil, soybean oil, mineral oil, sesame oil. Other carriers include saline, gum acacia, gelatin, starch paste, talc, keratin, colloidal silica, urea. In addition, auxiliary, stabilizing, thickening, lubricating and coloring agents are sometimes used. In one embodiment, when administered to a patient, the Ligand-Drug Conjugate or compositions thereof and pharmaceutically acceptable carriers are sterile.

Water is an exemplary carrier when the compounds are administered intravenously. Saline solutions and aqueous dextrose and glycerol solutions are often employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol. The present compositions, if desired, also contain minor amounts of wetting or emulsifying agents, or pH buffering agents.

In an embodiment, the conjugates are formulated in accordance with routine procedures as a pharmaceutical composition adapted for intravenous administration to animals, particularly human beings. Typically, the carriers or vehicles for intravenous administration are sterile isotonic aqueous buffer solutions. Where necessary, the compositions include a solubilizing agent. Compositions for intravenous administration optionally comprise a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachets indicating the quantity of active agent. Where a conjugate is to be administered by infusion, it is typically dispensed, for example, with an infusion bottle containing sterile pharmaceutical grade water or saline. In some aspects, where the conjugate is administered by injection, an ampoule of sterile water for injection or saline is sometimes provided and the ingredients are mixed prior to administration.

The pharmaceutical compositions are generally formulated as sterile, substantially isotonic and in full compliance with all Good Manufacturing Practice (GMP) regulations of the U.S. Food and Drug Administration.

### Methods of Preparing Drug-Linker Compounds and Ligand-Drug Conjugate Compounds

Provided herein are methods for preparing Drug-Linker compounds from the auristatin compounds provided herein. In addition to the methods recited below, method of preparing Drug-Linker compounds from auristatin compounds will be well known to a person of skill in the art.

Provided herein are methods for preparing Ligand-Drug Conjugate compounds from the auristatin compounds and Drug-Linker compounds provided herein. Methods for preparing the Ligand-Drug Conjugate compounds will be well known to a person of skill in the art.

### EXAMPLES

General Information Chemical Synthesis. All commercially available anhydrous solvents and reagents were used without further purification. UPLC-MS system 1 consisted of a Waters SQ mass detector 2 interfaced to an Acquity Ultra Performance LC equipped with a CORTECS UPLC C18 2.1×50 mm, 1.6 μm reverse phase column. The acidic mobile phase (0.1% formic acid) consisted of a gradient of 3% acetonitrile/97% water to 100% acetonitrile (flow rate=0.5 mL/min). Preparative HPLC was carried out on a Waters 2545 Binary Gradient Module with a Waters 2998 Photodiode Array Detector. Products were purified over a C12 Phenomenex Synergi 250×10.0 mm, 4 μm, 80 Å reverse phase column (<10 mg scale) or a C12 Phenomenex Synergi 250×50 mm, 10 μm, 80 Å reverse phase column (10-100 mg scale) eluting with 0.1% trifluoroacetic acid in water (solvent A) and 0.1% trifluoroacetic acid in acetonitrile (solvent B). The purification methods generally consisted of linear gradients of solvent A to solvent B, ramping from 90% aqueous solvent A to 5% solvent A. The flow rate was 4.6 mL/min with monitoring at 220 nm. NMR spectral data were collected on a Varian Mercury 400 MHz spectrometer. Coupling constants (J) are reported in hertz.

Throughout the Examples, reference is made to the following structures:

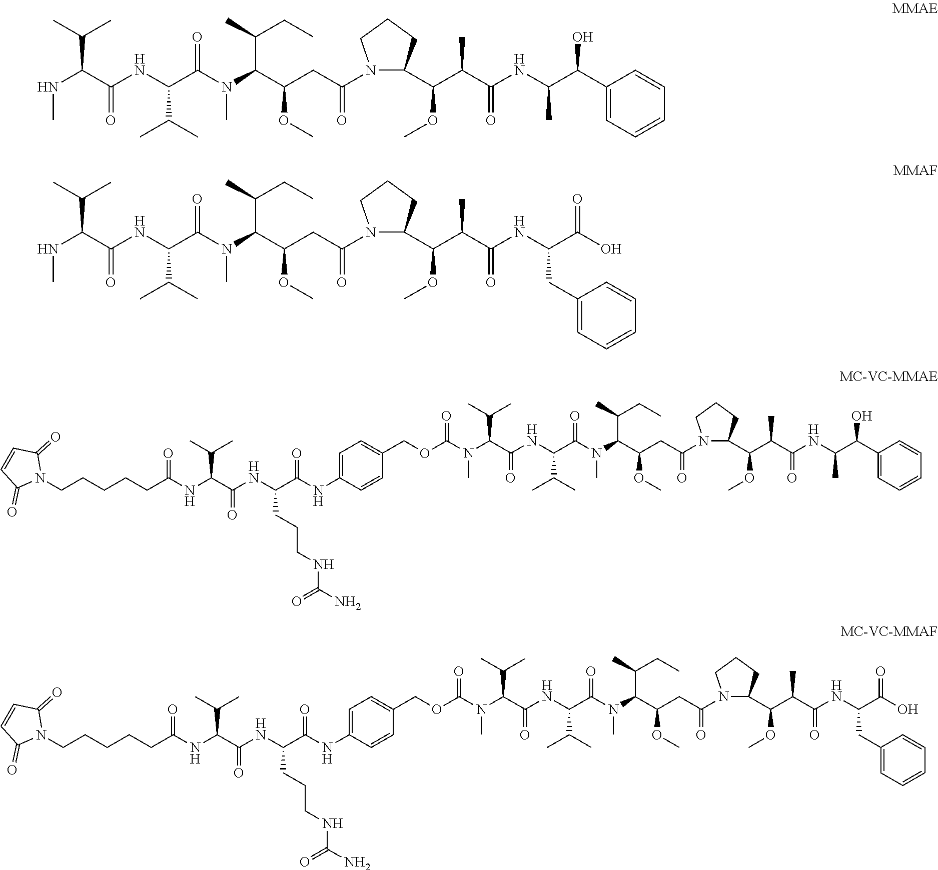

### Example 1. General Procedure for Solid-Phase/Solution-Phase Synthesis of Auristatins

A similar procedure for Fmoc deprotection, amide coupling, alkylation and resin cleavage has been published (Moquist, P. N. et al. Mol. Cancer Ther. 2021, 20, 320-328).

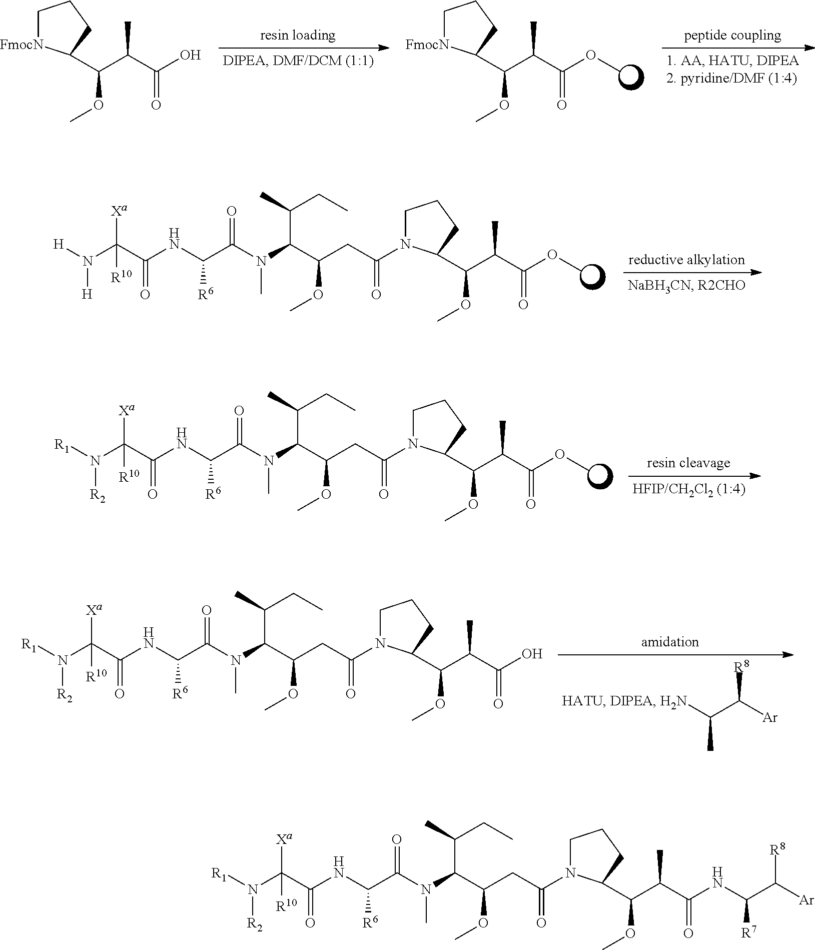

### Example 2. General Procedure for Resin Loading

2-chlorotrityl resin (100-200 mesh), 1% DVB (Millipore Sigma, 855017) was loaded into a vessel and DCM (5V) was added to swell the resin for 30 min. The solvent was removed, and the resin was treated with solution of Fmoc-Dap (1 equivalent to resin loading) in DMF and DCM (1:1, 10V) and shaken for 2 h. The solution was filtered, and the resin resin washed with DMF (3×3 min), DCM (3×3 min), and Et2O (3×3 min) and vacuum-dried in a desiccator.

### Example 3. General Procedure for Fmoc Deprotection

The resin was treated with a solution of piperidine in DMF (1:4 V/V) and shaken for 30 min. The solution was removed, and the resin was resubjected three additional times to the same conditions. The solution was filtered, and the resin are washed with DMF (3×3 min).

### Example 4. General Procedure for Amide Coupling

Fmoc-amino acid (2 equiv) was dissolved in dry DMF (0.2 M final concentration). DIPEA (4 equiv) and HATU (1.9 equiv) were added successively, and the reaction was stirred for 5 min. The resin was treated with the solution of activated Fmoc-amino acid and shaken for 2 h. The solution was filtered, and the resin was washed with DMF (3×3 min).

### Example 5. General Procedure for Resin Cleavage

Prior to cleavage the resin was washed with DCM (3×3 min). Resin was cleaved with a solution of 20:80 (V/V) solution of HFIP/DCM (10 min). Solution is removed in vacuo, and crude was dissolved for UPLC analysis and preparative HPLC. HPLC fraction were frozen and dried by lyophilization.

### Example 6. General Procedure for Reductive Alkylation

Aldehyde (5 equiv.) was dissolved in a 0.6 mL solution of 1% (V/V) AcOH in DMF, followed by the addition of NaBH3CN (4 equiv.). The resin was treated with the solution and shaken for 2 h. The solution was filtered, and the resin was washed with DMF (3×3 mL) and DCM (3×3 mL) and vacuum-dried in a desiccator.

### Example 7. General Procedure for C-Terminal Amine Coupling

Tetrapeptide (1 equiv) was dissolved in dry DMF (0.3 M final concentration). DIPEA (4 equiv) and HATU (0.95 equiv) were added successively, and the reaction was stirred for 15 min. The P5 amine was added to the activated ester and the reaction was stirred for 2 h. The solvent was removed in vacuo and purified by preparative HPLC. HPLC fractions were frozen and dried by lyophilization.

### Auristatin Compounds

Examples 8 to 33 were synthesized according to the procedures of Examples 1 to 7.

### Example 8

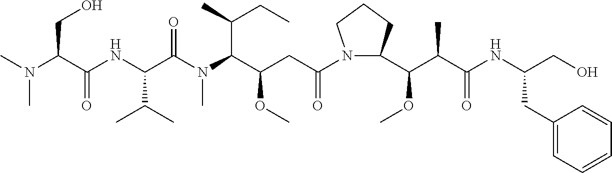 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)N(C)C)C(C)C]

Compound 1.1 was prepared by dimethylation of tetrapeptide and amide coupling of tetrapeptide with L-phenylalaninol. Analytical UPLC-MS: t_{R}=1.33 min, m/z (ES+) 720.49 (M+H)⁺, found 720.85.

### Example 9

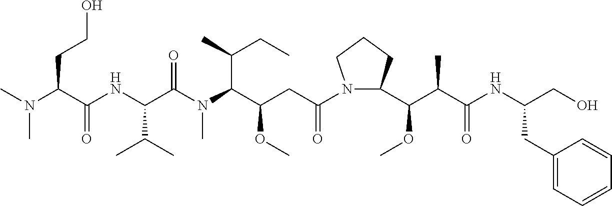 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CCO)N(C)C)C(C)C]

Compound 1.3 was prepared by dimethylation of tetrapeptide and amide coupling of tetrapeptide with L-phenylalaninol. Analytical UPLC-MS: t_{R}=1.16 min, m/z (ES+) 734.50 (M+H)⁺, found 734.49.

### Example 10

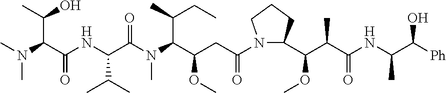 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H]([C@@H](C)O)N(C)C)C(C)C]

Compound 1.4 was prepared by dimethylation of tetrapeptide and amide coupling of tetrapeptide with (1S,2R)-(+)-norephedrine. Analytical UPLC-MS: t_{R}=1.29 min, m/z (ES+) 734.50 (M+H)⁺, found 734.48.

### Example 11

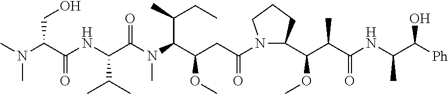 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](CO)N(C)C)C(C)C]

Compound 1.2 was prepared by dimethylation of tetrapeptide and amide coupling of tetrapeptide with (1S,2R)-(+)-norephedrine. Analytical UPLC-MS: t_{R}=1.26 min, m/z (ES+) 720.48 (M+H)+, found 720.51.

### Example 12

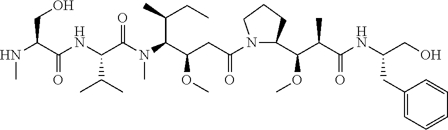 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)NC)C(C)C]

Compound 1.9 was prepared by amide coupling of tetrapeptide with L-phenylalaninol. Analytical UPLC-MS: t_{R}=1.30 min, m/z (ES+) 706.47 (M+H)+, found 707.62.

### Example 13

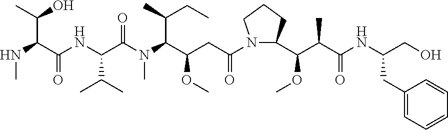 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](NC)[C@@H](C)O)C(C)C]

Compound 1.12 was prepared by amide coupling of tetrapeptide with L-phenylalaninol. Analytical UPLC-MS: t_{R}=1.40 min, m/z (ES+) 719.48 (M+H)+, found 720.99.

### Example 14

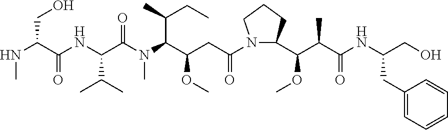 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](CO)NC)C(C)C]

Compound 1.10 was prepared by amide coupling of tetrapeptide with L-phenylalaninol. Analytical UPLC-MS: t_{R}=1.27 min, m/z (ES+) 706.47 (M+H)+, found 706.57.

### Example 15

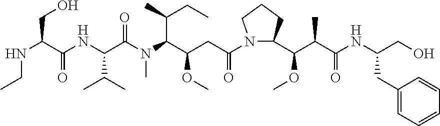 [SMILES: CCN[C@@H](CO)C(=O)N[C@H](C(=O)N(C)[C@@H]([C@@H](C)CC)[C@@H](CC(=O)N1CCC[C@H]1[C@H](OC)[C@@H](C)C(=O)N[C@H](CO)Cc1ccccc1)OC)C(C)C]

Compound 1.13 was prepared by reductive alkylation of tetrapeptide with acetaldehyde followed by amide coupling of tetrapeptide with L-phenylalaninol. Analytical UPLC-MS: t_{R}=1.21 min, m/z (ES+) 720.49 (M+H)⁺, found 720.57.

### Example 16

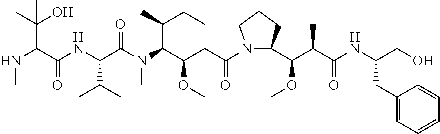 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)C(NC)C(C)(C)O)C(C)C]

Compound 1.11 was prepared by amide coupling of tetrapeptide with L-phenylalaninol. Analytical UPLC-MS: t_{R}=1.47 min, m/z (ES+) 734.51 (M+H)+, found 734.85.

### Example 17

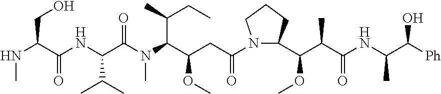 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](CO)NC)C(C)C]

Compound 1.29 was prepared by amide coupling of tetrapeptide with (1S,2R)-(+)-norephedrine. Analytical UPLC-MS: t_{R}=1.58 min, m/z (ES+) 728.46 (M+Na)+, found 728.73.

### Example 18

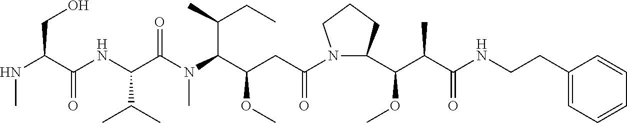 [SMILES: CC[C@H](C)[C@@H]([C@@H](CC(=O)N1CCC[C@H]1[C@H](OC)[C@@H](C)C(=O)NCCc1ccccc1)OC)N(C)C(=O)[C@@H](NC(=O)[C@H](CO)NC)C(C)C]

Compound 1.30 was prepared by amide coupling of tetrapeptide with phenethylamine. Analytical UPLC-MS: t_{R}=1.70 min, m/z (ES+) 698.45 (M+Na)+, found 698.50.

### Example 19

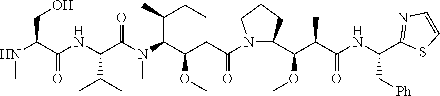 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H](CO)NC)C(C)C]

Compound 1.32 was prepared by amide coupling of tetrapeptide with S-dolaphenine. Analytical UPLC-MS: t_{R}=1.85 min, m/z (ES+) 759.45 (M+H)⁺, found 760.05.

### Example 20

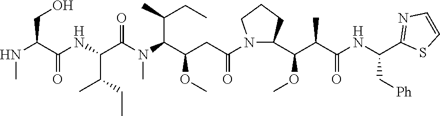 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H](CO)NC)[C@@H](C)CC]

Compound 1.33 was prepared by amide coupling of tetrapeptide with S-dolaphenine. Analytical UPLC-MS: tR=1.89 min, m/z (ES+) 773.46 (M+H)⁺, found 773.58.

### Example 21

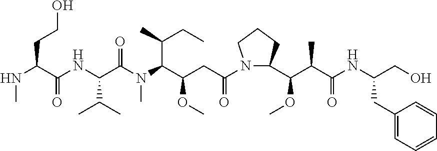 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CCO)NC)C(C)C]

N-terminal amino acid Fmoc-(N-methyl)-L-h-serine was synthesized from L-h-Serine(OtBu)-OH by reaction with formaldehyde (37% aqueous, 1.5 equiv), NaH₂PO₄ (2 equiv) in water, Zn (dust, 2 equiv), and AcOH (1 equiv.) at RT overnight similar to known procedure (Da Silva, R. A. et al. Tetrahedron Lett. 2007, 48, 7680-7682). Fmoc protecting group was then installed as described in a reported procedure (Wuts, P; Green, T. “Protective Groups in Organic Synthesis” New York, 2006).

Compound 1.14 was prepared by amide coupling of tetrapeptide with L-phenylalaninol. Analytical UPLC-MS: t_{R}=1.13 min, m/z (ES+) 720.45 (M+H)+, found 720.49.

### Example 22

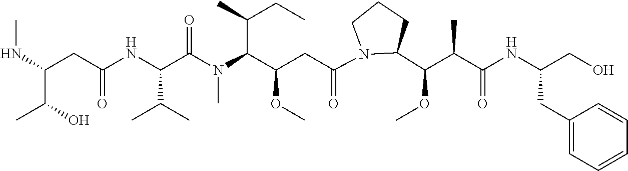 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)C[C@@H](NC)[C@@H](C)O)C(C)C]

N-terminal amino acid Fmoc-(N-methyl)-βh-threonine was synthesized from βh-threonine-(OtBu)-OH by reaction with formaldehyde (37% aqueous, 1.5 equiv), NaH₂PO₄ (2 equiv) in water, Zn (dust, 2 equiv) and AcOH (1 equiv.) at RT overnight similar to known procedure (Da Silva, R. A. et al. Tetrahedron Lett. 2007, 48, 7680-7682). Fmoc protecting group was then installed as described in a reported procedure (Wuts, P; Green, T. “Protective Groups in Organic Synthesis” New York, 2006).

Compound 1.58 was prepared by amide coupling of tetrapeptide with L-phenylalaninol. Analytical UPLC-MS: t_{R}=1.20 min, m/z (ES+) 734.50 (M+H)+, found 734.52.

### Example 23

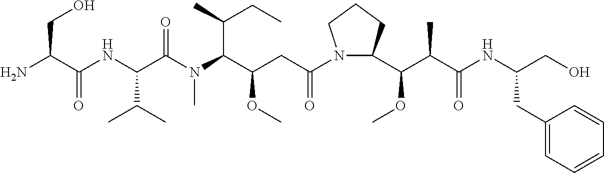 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](N)CO)C(C)C]

Compound 1.16 was prepared by amide coupling of tetrapeptide with L-phenylalaninol. Analytical UPLC-MS: t_{R}=1.38 min, m/z (ES+) 692.46 (M+H)+, found 692.93.

### Example 24

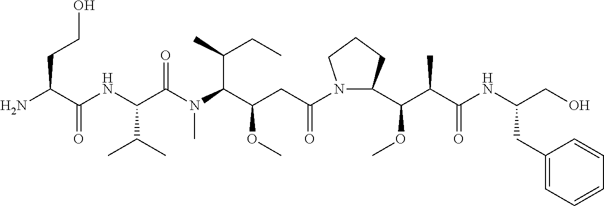 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](N)CCO)C(C)C]

Compound 1.18 was prepared by amide coupling of tetrapeptide with L-phenylalaninol. Analytical UPLC-MS: t_{R}=1.37 min, m/z (ES+) 706.47 (M+H)+, found 706.55.

### Example 25

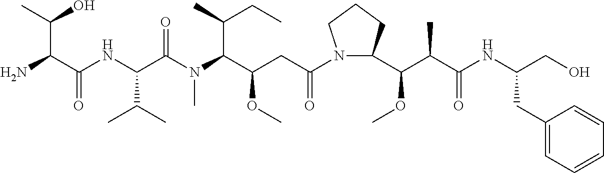 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](N)[C@@H](C)O)C(C)C]

Compound 1.20 was prepared by amide coupling of tetrapeptide with L-phenylalaninol. Analytical UPLC-MS: t_{R}=1.30 min, m/z (ES+) 706.47 (M+H)+, found 706.39.

### Example 26

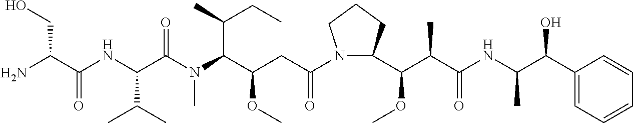 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](N)CO)C(C)C]

Compound 1.72 was prepared by amide coupling of tetrapeptide with (1S,2R)-(+)-norephedrine. Analytical UPLC-MS: t_{R}=1.19 min, m/z (ES+) 692.46 (M+H)+, found 692.49.

### Example 27

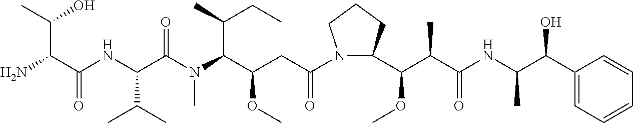 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](N)[C@H](C)O)C(C)C]

Compound 1.22 was prepared by amide coupling of tetrapeptide with (1S,2R)-(+)-norephedrine. Analytical UPLC-MS: t_{R}=1.25 min, m/z (ES+) 706.47 (M+H)+, found 706.56.

### Example 28

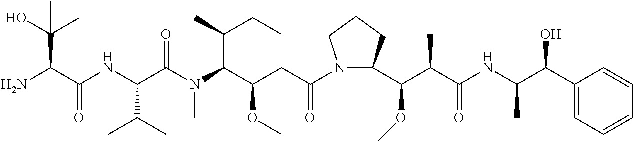 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](N)C(C)(C)O)C(C)C]

Compound 1.23 prepared by amide coupling of tetrapeptide with (1S,2R)-(+)-norephedrine. Analytical UPLC-MS: t_{R}=1.28 min, m/z (ES+) 720.48 (M+H)+, found 720.76.

### Example 29

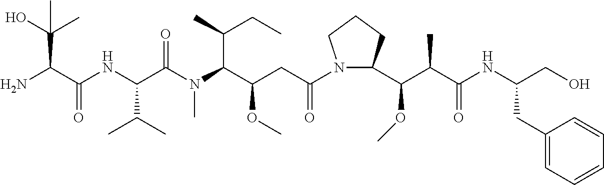 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](N)C(C)(C)O)C(C)C]

Compound 1.21 was prepared by amide coupling of tetrapeptide with L-phenylalaninol. Analytical UPLC-MS: t_{R}=1.30 min, m/z (ES+) 720.49 (M+H)+, found 720.85.

### Example 30

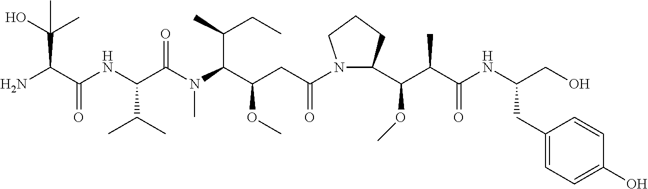 [SMILES: CC[C@H](C)[C@@H]([C@@H](CC(=O)N1CCC[C@H]1[C@H](OC)[C@@H](C)C(=O)N[C@H](CO)Cc1ccc(O)cc1)OC)N(C)C(=O)[C@@H](NC(=O)[C@@H](N)C(C)(C)O)C(C)C]

Compound 1.24 was prepared by amide coupling of tetrapeptide with L-tyrosinol. Analytical UPLC-MS: t_{R}=1.15 min, m/z (ES+) 736.49 (M+H)+, found 736.79.

### Example 31

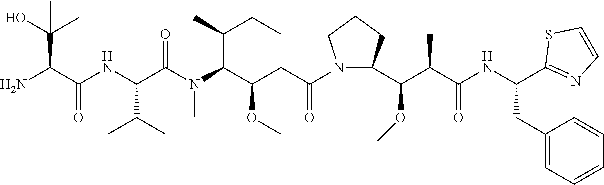 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H](N)C(C)(C)O)C(C)C]

Compound 1.25 was prepared by amide coupling of tetrapeptide with S-dolaphenine. Analytical UPLC-MS: t_{R}=1.49 min m/z (ES+) 773.04 (M+H)+, found 773.61.

### Example 32

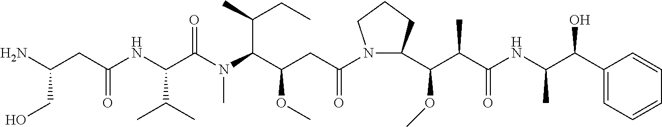 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)C[C@@H](N)CO)C(C)C]

Compound 1.56 was prepared by amide coupling of tetrapeptide with (1S,2R)-(+)-norephedrine. Analytical UPLC-MS: t_{R}=1.25 min, m/z (ES+) 706.48 (M+H)+, found 706.80.

### Example 33

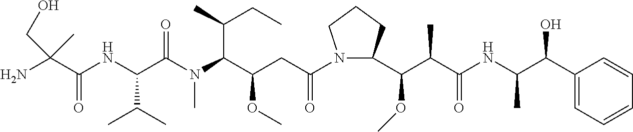 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)C(C)(N)CO)C(C)C]

Compound 1.59 was prepared by amide coupling of tetrapeptide with (1S,2R)-(+)-norephedrine. Analytical UPLC-MS: t_{R}=1.30 min, m/z (ES+) 706.48 (M+H)+, found 706.71.

### Example 34

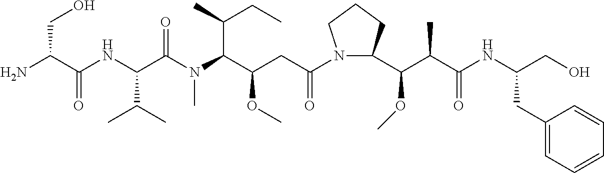 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](N)CO)C(C)C]

Compound 1.19 can be prepared by amide coupling of tetrapeptide with L-phenylalaninol.

### Example 35

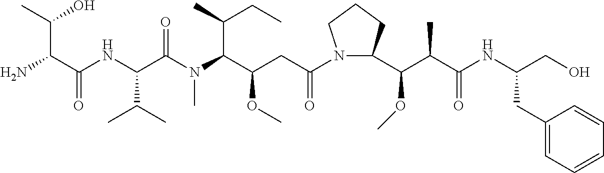 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](N)[C@H](C)O)C(C)C]

Compound 1.17 can be prepared by amide coupling of tetrapeptide with L-phenylalaninol.

### Example 36. General Procedure to Synthesize Cyclic Carbamate Auristatins

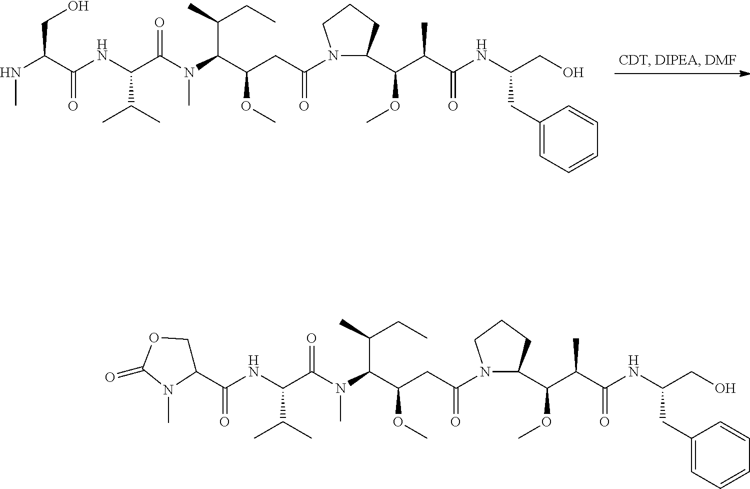 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)NC)C(C)C ; CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)C1COC(=O)N1C)C(C)C]

Auristatin (1 equiv) was dissolved in DMF (0.3 M final concentration). DIPEA (5 equiv) and CDI (4 equiv) were added successively, and the reaction was stirred for 2 h. Upon reaction completion, a solution of LiOH was added to hydrolyze any undesired carbonate formation. The organic lay was separated, and the solvent was removed in vacuo. The crude mixture was purified by preparative HPLC. HPLC fractions were frozen and dried by lyophilization.

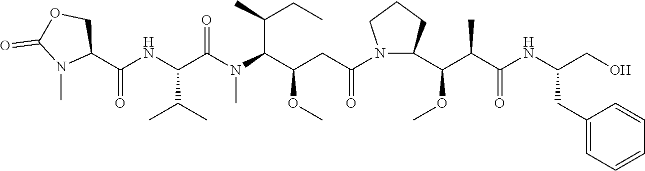 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H]1COC(=O)N1C)C(C)C]

Compound 1.50. Analytical UPLC-MS: t_{R}=1.67 min, m/z (ES+) 732.45 (M+H)+, found 732.99.

### Example 37

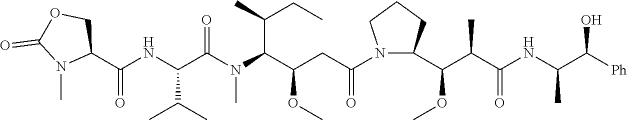 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H]1COC(=O)N1C)C(C)C]

Compound 1.53. Analytical UPLC-MS: t_{R}=2.04 min, m/z (ES+) 732.45 (M+H)+, found 732.90.

### Example 38

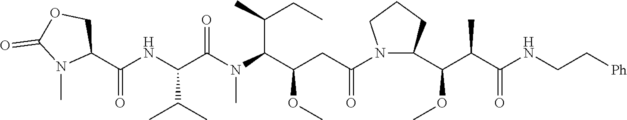 [SMILES: CC[C@H](C)[C@@H]([C@@H](CC(=O)N1CCC[C@H]1[C@H](OC)[C@@H](C)C(=O)NCCc1ccccc1)OC)N(C)C(=O)[C@@H](NC(=O)[C@@H]1COC(=O)N1C)C(C)C]

Compound 1.54. Analytical UPLC-MS: t_{R}=2.23 min, m/z (ES+) 702.44 (M+H)+, found 702.52.

### Example 39

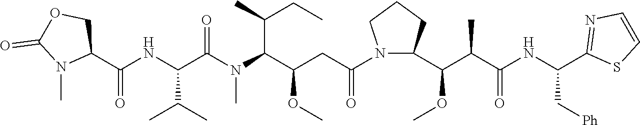 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1COC(=O)N1C)C(C)C]

Compound 1.55. Analytical UPLC-MS: t_{R}=2.04 min, m/z (ES+) 785.43 (M+H)+, found 785.48.

### Example 40

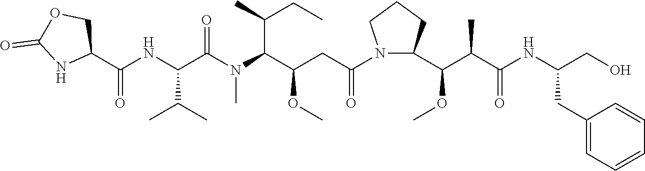 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H]1COC(=O)N1)C(C)C]

Compound 1.46. Analytical UPLC-MS: t_{R}=1.57 min, m/z (ES+) 718.44 (M+H)+, found 718.84.

### Example 41

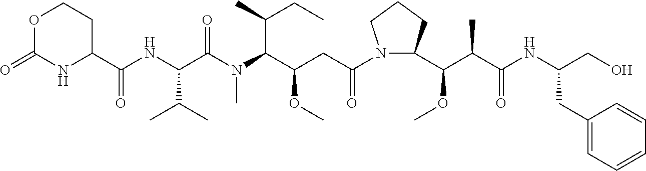 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)C1CCOC(=O)N1)C(C)C]

Compound 1.47. Analytical UPLC-MS: t_{R}=1.58 min, m/z (ES+) 732.45 (M+H)+, found 732.99.

### Example 42

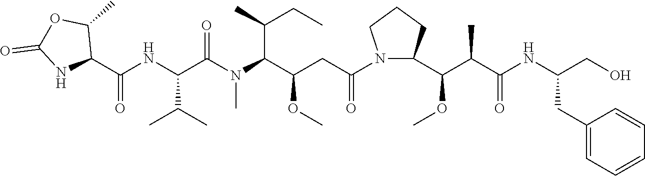 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H]1NC(=O)O[C@@H]1C)C(C)C]

Compound 1.48. Analytical UPLC-MS: t_{R}=1.70 min, m/z (ES+) 732.45 (M+H)+, found 732.73.

### Example 43

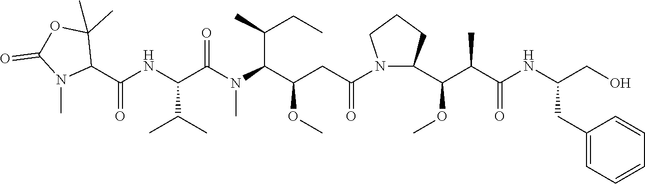 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)C1N(C)C(=O)OC1(C)C)C(C)C]

Compound 1.60. Analytical UPLC-MS: t_{R}=1.78 min, m/z (ES+) 760.49 (M+H)+, found 760.85.

### Example 44

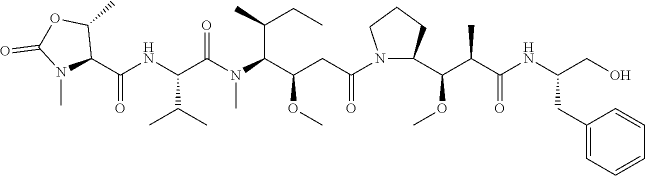 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H]1[C@@H](C)OC(=O)N1C)C(C)C]

Compound 1.51. Analytical UPLC-MS: t_{R}=1.70 min, m/z (ES+) 732.45 (M+H)+, found 732.73.

### Example 45

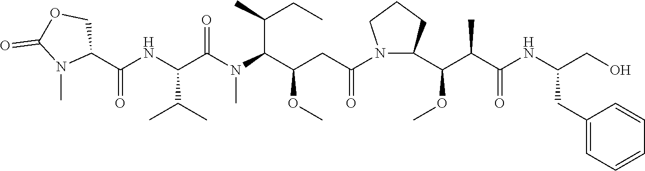 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H]1COC(=O)N1C)C(C)C]

Compound 1.52. Analytical UPLC-MS: t_{R}=1.61 min, m/z (ES+) 732.45 (M+H)+, found 732.55.

### Example 46

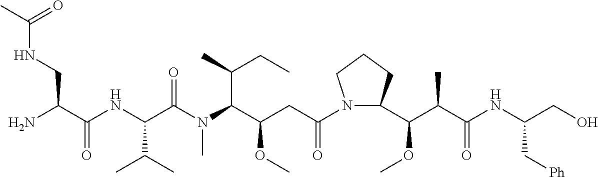 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](N)CNC(C)=O)C(C)C]

Compound 1.61 was prepared by amide coupling of tetrapeptide with L-phenylalaninol. Analytical UPLC-MS (Method 1): t_{R}=1.25 min, m/z (ES+) 733.48 (M+H)+, found 733.78.

### Example 47

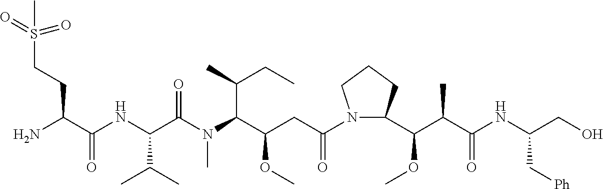 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](N)CCS(C)(=O)=O)C(C)C]

Compound 1.28 was prepared by amide coupling of tetrapeptide with L-phenylalaninol. Analytical UPLC-MS (Method 1): t_{R}=1.24 min, m/z (ES+) 768.45 (M+H)+, found 768.60.

### Example 48

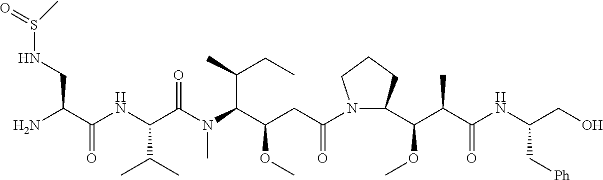 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](N)CNS(C)=O)C(C)C]

Compound 1.62 was prepared by amide coupling of tetrapeptide with L-phenylalaninol. Analytical UPLC-MS (Method 1): t_{R}=1.20 min, m/z (ES+) 752.46 (M+H)+, found 752.19.

### Example 49

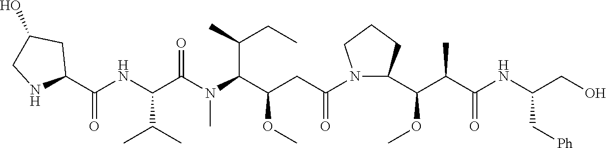 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H]1C[C@@H](O)CN1)C(C)C]

Compound 1.41 was prepared by amide coupling of tetrapeptide with L-phenylalaninol. Analytical UPLC-MS (Method 1): t_{R}=1.21 min, m/z (ES+) 718.47 (M+H)+, found 718.10.

### Example 50

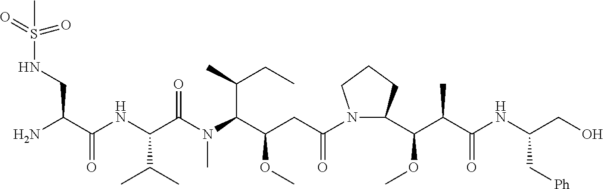 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](N)CNS(C)(=O)=O)C(C)C]

Compound 1.64 was prepared by amide coupling of tetrapeptide with L-phenylalaninol. Analytical UPLC-MS (Method 1): t_{R}=1.36 min, m/z (ES+) 769.45 (M+H)+, found 769.20.

### Example 51

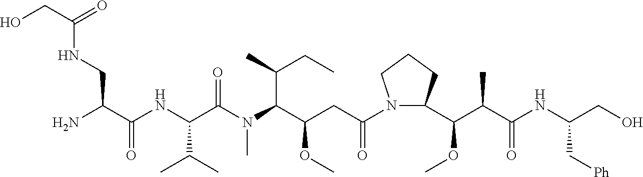 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](N)CNC(=O)CO)C(C)C]

Compound 1.65 was prepared by amide coupling of tetrapeptide with L-phenylalaninol. Analytical UPLC-MS (Method 1): t_{R}=1.21 min, m/z (ES+) 749.47 (M+H)+, found 749.27.

### Example 52

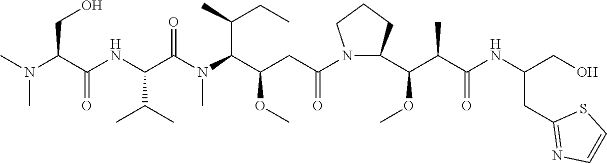 [SMILES: CC[C@H](C)[C@@H]([C@@H](CC(=O)N1CCC[C@H]1[C@H](OC)[C@@H](C)C(=O)NC(CO)Cc1nccs1)OC)N(C)C(=O)[C@@H](NC(=O)[C@H](CO)N(C)C)C(C)C]

Compound 1.66 was prepared by amide coupling of tetrapeptide with L-phenylalaninol. Analytical UPLC-MS (Method 1): t_{R}=1.09 min, m/z (ES+) 727.43 (M+H)+, found 727.41.

### Example 53

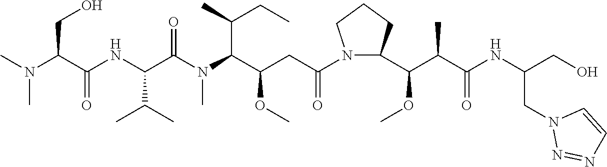 [SMILES: CC[C@H](C)[C@@H]([C@@H](CC(=O)N1CCC[C@H]1[C@H](OC)[C@@H](C)C(=O)NC(CO)Cn1ccnn1)OC)N(C)C(=O)[C@@H](NC(=O)[C@H](CO)N(C)C)C(C)C]

Compound 1.67 was prepared by amide coupling of tetrapeptide with 2-amino-3-(1H-1,2,3-triazol-1-yl)propan-1-ol. Analytical UPLC-MS (Method 1): t_{R}=1.04 min, m/z (ES+) 711.47 (M+H)⁺, found 711.19.

### Example 54

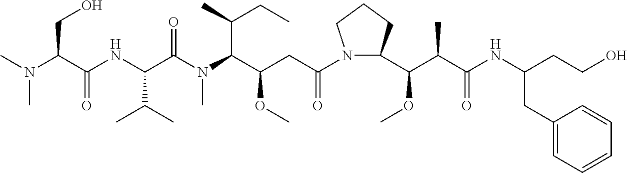 [SMILES: CC[C@H](C)[C@@H]([C@@H](CC(=O)N1CCC[C@H]1[C@H](OC)[C@@H](C)C(=O)NC(CCO)Cc1ccccc1)OC)N(C)C(=O)[C@@H](NC(=O)[C@H](CO)N(C)C)C(C)C]

Compound 1.68 was prepared by amide coupling of tetrapeptide with 3-amino-4-phenylbutan-1-ol. Analytical UPLC-MS (Method 1): t_{R}=1.28 min, m/z (ES+) 734.50 (M+H)⁺, found 734.25.

### Example 55

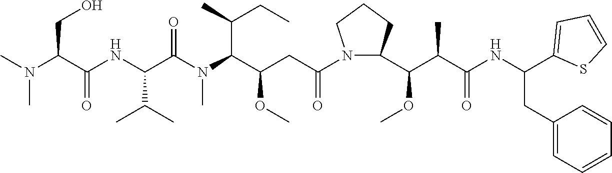 [SMILES: CC[C@H](C)[C@@H]([C@@H](CC(=O)N1CCC[C@H]1[C@H](OC)[C@@H](C)C(=O)NC(Cc1ccccc1)c1cccs1)OC)N(C)C(=O)[C@@H](NC(=O)[C@H](CO)N(C)C)C(C)C]

Compound 1.69 was prepared by amide coupling of tetrapeptide with 2-phenyl-1-(thiophen-2-yl)ethan-1-amine. Analytical UPLC-MS (Method 1): t_{R}=1.53 min, m/z (ES+) 772.46 (M+H)⁺, found 772.19.

### Example 56

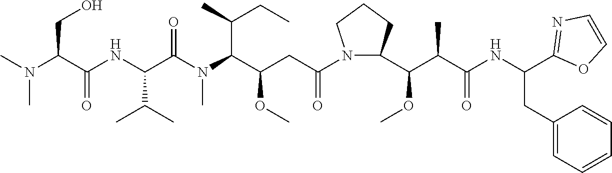 [SMILES: CC[C@H](C)[C@@H]([C@@H](CC(=O)N1CCC[C@H]1[C@H](OC)[C@@H](C)C(=O)NC(Cc1ccccc1)c1ncco1)OC)N(C)C(=O)[C@@H](NC(=O)[C@H](CO)N(C)C)C(C)C]

Compound 1.70 was prepared by amide coupling of tetrapeptide with 1-(oxazol-2-yl)-2-phenylethan-1-amine. Analytical UPLC-MS (Method 1): t_{R}=1.39 min, m/z (ES+) 757.48 (M+H)⁺, found 757.24.

### Example 57

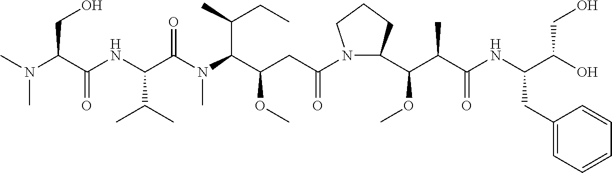 [SMILES: CC[C@H](C)[C@@H]([C@@H](CC(=O)N1CCC[C@H]1[C@H](OC)[C@@H](C)C(=O)N[C@@H](Cc1ccccc1)[C@@H](O)CO)OC)N(C)C(=O)[C@@H](NC(=O)[C@H](CO)N(C)C)C(C)C]

Compound 1.71 was prepared by amide coupling of tetrapeptide with (2R,3S)-3-amino-4-phenylbutane-1,2-diol. Analytical UPLC-MS (Method 1): t_{R}=1.18 min, m/z (ES+) 750.49 (M+H)⁺, found 750.20.

### Comparative Example A

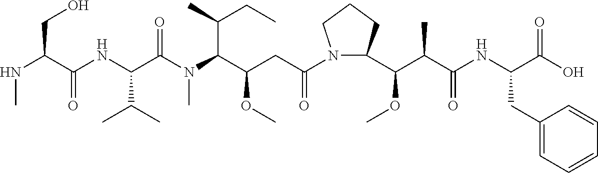 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](CO)NC)C(C)C]

Comparative Example A was prepared by amide coupling of tetrapeptide with L-phenylalanine. Analytical UPLC-MS (Method 1): t_{R}=1.34 min, m/z (ES+) 720.45 (M+H)+, found 720.38.

### Comparative Example B

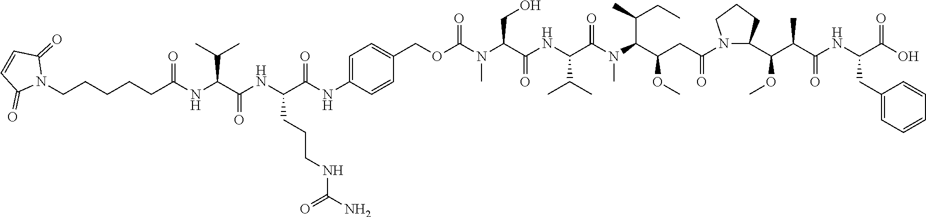 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](CO)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

Comparative Example B was prepared using method A. Analytical UPLC-MS (Method 1): t_{R}=1.79 min, m/z (ES+) 1318.73 (M+H)+, found 1319.21.

### Comparative Example C

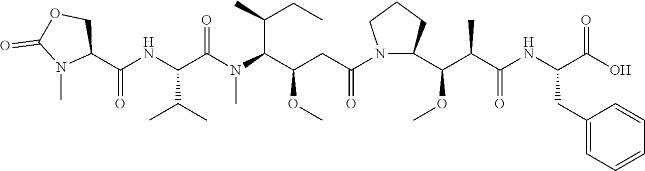 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H]1COC(=O)N1C)C(C)C]

Comparative Example C was prepared in a similar fashion to Example 36. Analytical UPLC-MS (Method 1): t_{R}=1.73 min, m/z (ES+) 746.43 (M+H)+, found 746.76.

### General Procedures for Solution Phase Synthesis of Auristatins

### Example 58. General Procedure for Synthesis of Dipeptide Carbamate Linkers (Method A

### Step 1. Synthesis of MC-VC-PAB-OPFP

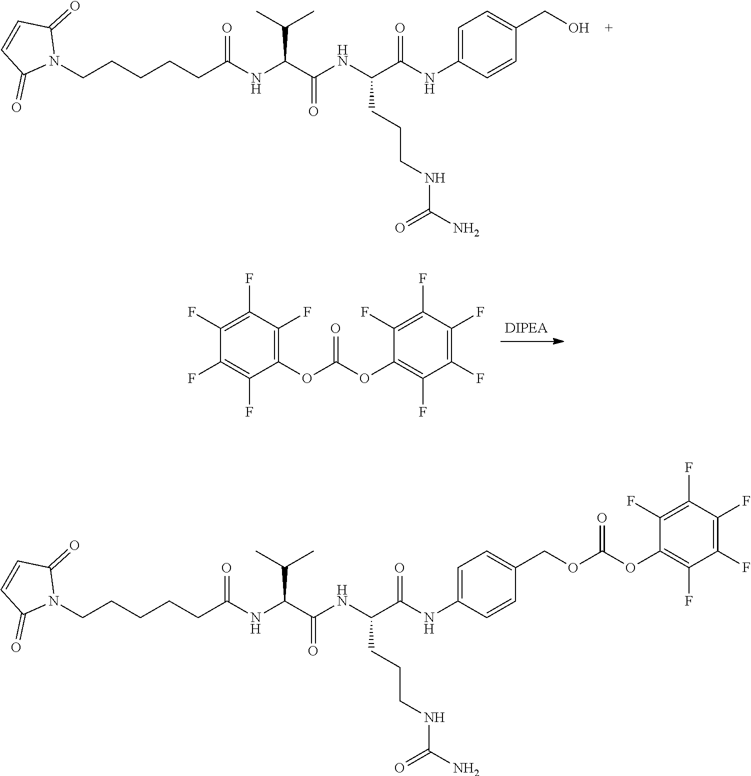 [SMILES: CC(C)[C@H](NC(=O)CCCCCN1C(=O)C=CC1=O)C(=O)N[C@@H](CCCNC(N)=O)C(=O)Nc1ccc(CO)cc1 ; O=C(Oc1c(F)c(F)c(F)c(F)c1F)Oc1c(F)c(F)c(F)c(F)c1F ; CC(C)[C@H](NC(=O)CCCCCN1C(=O)C=CC1=O)C(=O)N[C@@H](CCCNC(N)=O)C(=O)Nc1ccc(COC(=O)Oc2c(F)c(F)c(F)c(F)c2F)cc1]

A 20 mL vial equipped with stir bar was charged with MC-VC-PABA (300 mg, 0.52 mmol), PFP carbonate (413 mg, 1.04 mmol), DMF (5 mL), and DIPEA (137 uL, 0.78 mmol). The mixture was stirred at RT for 3 hours. The solvent was removed in vacuo and redissolved in 20% piperidine in DMF (5 mL). The reaction was stirred for 1 hour. The solvent was removed in vacuo and was purified by Biotage reverse phase flash column chromatography with acetonitrile+0.05% TFA and water+0.05% TFA gradient up to 95% acetonitrile. The product fractions were lyophilized to afford 177 mg (43% yield). Analytical UPLC-MS: t_{R}=2.05 min, m/z (ES+) 783.27 (M+H)+, found 783.42.

### Step 2. Drug-Linker Formation

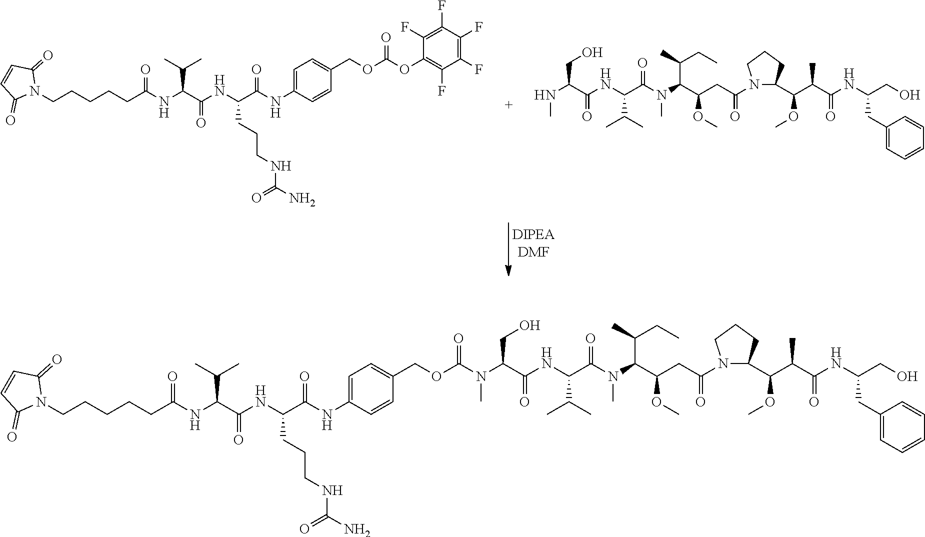 [SMILES: CC(C)[C@H](NC(=O)CCCCCN1C(=O)C=CC1=O)C(=O)N[C@@H](CCCNC(N)=O)C(=O)Nc1ccc(COC(=O)Oc2c(F)c(F)c(F)c(F)c2F)cc1 ; CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)NC)C(C)C ; CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

A 20 mL vial equipped with stir bar was charged with the compound of Example 12 (70 mg, 0.1 mmol), MC-VC-PAB-OPFP (78 mg, 0.1 mmol), DMF (5 mL), and DIPEA (26 uL, 0.15 mmol). The mixture was stirred at RT overnight. The solvent was removed in vacuo and was purified by preparative HPLC. The product fractions were lyophilized to afford 80.7 mg (62% yield) of compound 2.2. Analytical UPLC-MS: t_{R}=1.82 min, m/z (ES+) 1304.75 (M+H)+, found 1305.54.

### Example 59. General Procedure for Synthesis of (D)Leu-Ala-Glu Tripeptide Carbamate Drug-Linkers (Method B

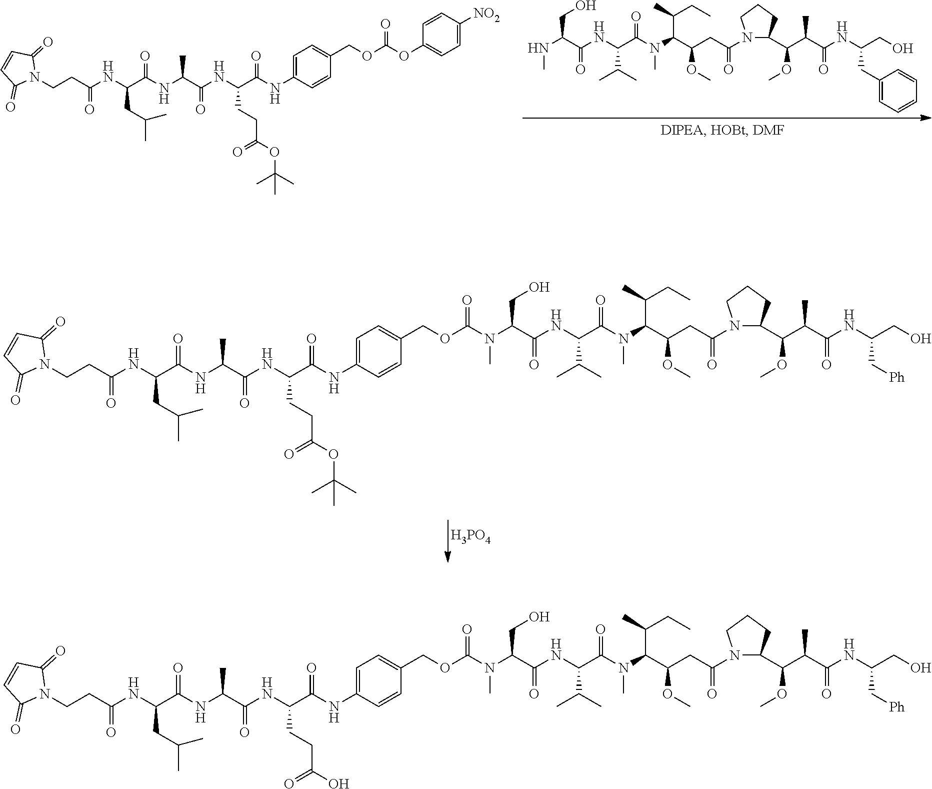

This is a modified version of a previously reported procedure. See WO 2021/055865.

A 4 mL vial equipped with stir bar was charged with the compound from Example 12 (100 mg, 0.14 mmol), MC-(D)-Leu-Ala-Glu(OtBu)-PABA-OₚNP (114 mg, 0.14 mmol), DMF (5 mL), DIPEA (51 uL, 0.28 mmol), and HOBt (37 mg, 0.28 mmol). The mixture was stirred at RT overnight. The solvent was removed in vacuo.

The crude mixture was dissolved in propionitrile (3 mL), and H₃PO₄ (3 mL) was slowly added to the reaction mixture at RT. The reaction mixture was stirred for 2 h and propionitrile (6 mL) was added to the mixture. The organic layer was separated, and the reaction was concentrated and purified by HPLC. The product fractions were lyophilized to afford 54 mg (26% yield) of compound 2.6.

Analytical UPLC-MS: t_{R}=1.71 min, m/z (ES+) 1319.71 (M+H)⁺, found 1320.06.

### Example 60. General Procedure for Synthesis of Quaternary Linkers (Method C

### Step 1. Synthesis of MC-VC-PAB-Cl

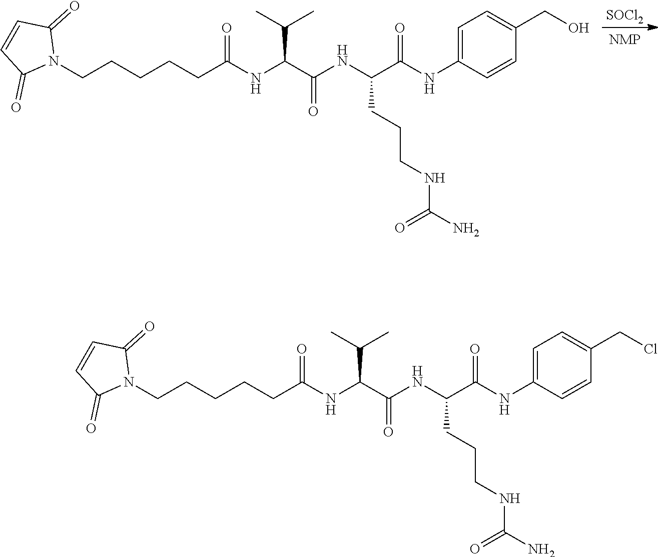

A 20 mL vial equipped with stir bar was charged with solution of MC-Val-Cit-PAB-OH (200 mg, 349.25 umol) in N-methyl-2-pyrrolidone (NMP, 3 mL) and then cooled to 0° C. A solution of SOCl₂ (70.64 mg, 593.73 umol) in NMP (0.5 mL) was added dropwise. The mixture was stirred at 20° C. for 30 min. TLC (ethyl acetate:methanol=3:1, Rf=0.5) showed the reaction was completed. The mixture was poured into ice water and filtered, washed with acetonitrile and ethyl acetate, dried in high vacuum to give MC-Val-Cit-PAB-Cl (100 mg, yield 48.4%). Analytical UPLC-MS: t_{R}=2.05 min, m/z (ES+) 591.27 (M+H)⁺, found 591.42. 1H NMR: (400 MHz, DMSO-d6) δ=10.16-9.89 (m, 1H), 8.08 (br d, J=7.4 Hz, 1H), 7.79 (br d, J=8.6 Hz, 1H), 7.60 (br d, J=8.5 Hz, 2H), 7.36 (d, J=8.5 Hz, 2H), 7.00 (s, 2H), 5.96 (br t, J=5.4 Hz, 1H), 5.52-5.20 (m, 2H), 4.71 (s, 2H), 4.45-4.31 (m, 1H), 4.19 (br t, J=7.6 Hz, 1H), 3.45-3.34 (m, 2H), 3.09-2.85 (m, 2H), 2.14 (tq, J=7.2, 14.5 Hz, 2H), 2.03-1.85 (m, 1H), 1.75-1.55 (m, 2H), 1.54-1.27 (m, 6H), 1.26-1.10 (m, 2H), 0.83 (br dd, J=6.8, 12.9 Hz, 6H).

### Step 2. Drug-Linker Formation

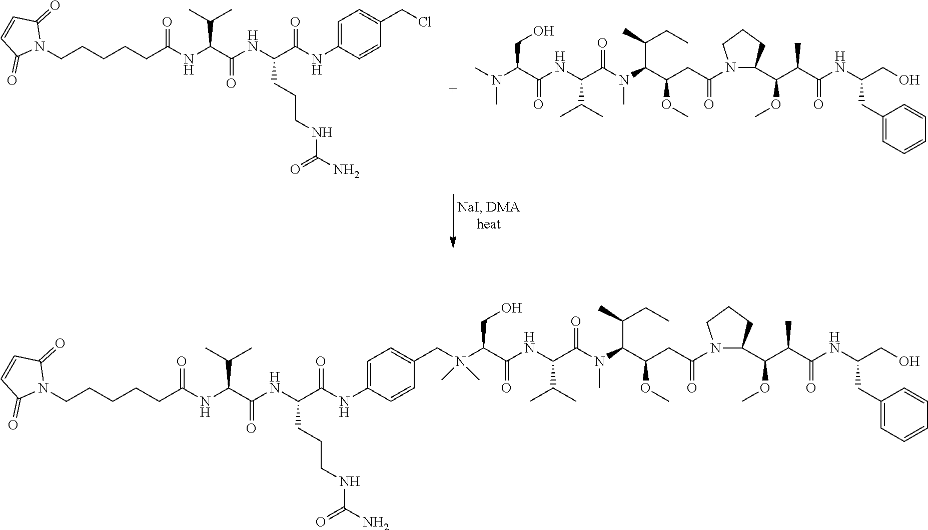

A 4 mL vial equipped with stir bar was charged with the compound of Example 8 (3.3 mg, 0.004 mmol), mc-VC-PAB-Cl (4.8 mg, 0.008 mmol), DMA (0.5 mL), and NaI (1.2 mg, 0.008 mmol). The mixture was stirred at 70° C. for 4 hours. The reaction was purified by preparative HPLC. The product fractions were lyophilized to afford 3.9 mg (71% yield) of the title compound (compound 2.1). Analytical UPLC-MS: t_{R}=1.51 min, m/z (ES+) 1275.62 (M)⁺, found 1275.46.

### Example 61

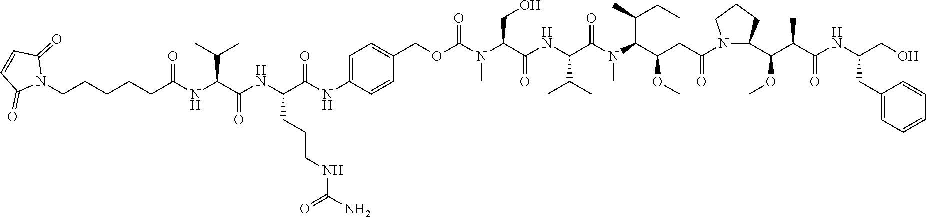 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

Compound 2.2 was synthesized using method A. 62% yield. Analytical UPLC-MS: t_{R}=1.82 min, m/z (ES+) 1304.75 (M+H)⁺, found 1305.54.

### Example 62

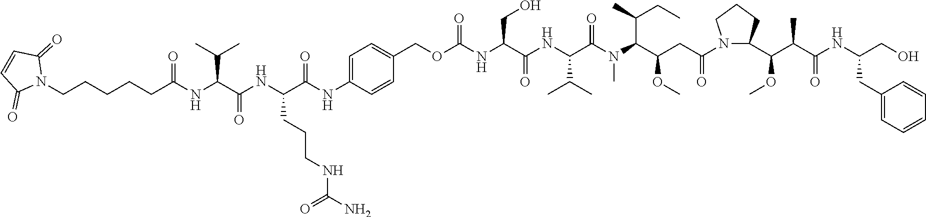 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)NC(=O)OCc1ccc(NC(=O)[C@H](CCCNC(N)=O)NC(=O)[C@@H](NC(=O)CCCCCN2C(=O)C=CC2=O)C(C)C)cc1)C(C)C]

Compound 2.9 was synthesized using method A. 43% yield. Analytical UPLC-MS: t_{R}=1.72 min, m/z (ES+) 1290.73 (M+H)+, found 1291.59.

### Example 63

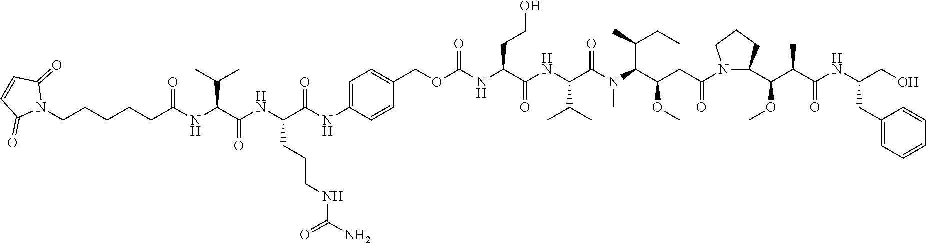 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CCO)NC(=O)OCc1ccc(NC(=O)[C@H](CCCNC(N)=O)NC(=O)[C@@H](NC(=O)CCCCCN2C(=O)C=CC2=O)C(C)C)cc1)C(C)C]

Compound 2.10 was synthesized using method A. 52% yield. Analytical UPLC-MS: t_{R}=1.74 min, m/z (ES+) 1304.75 (M+H)⁺, found 1305.28.

### Example 64

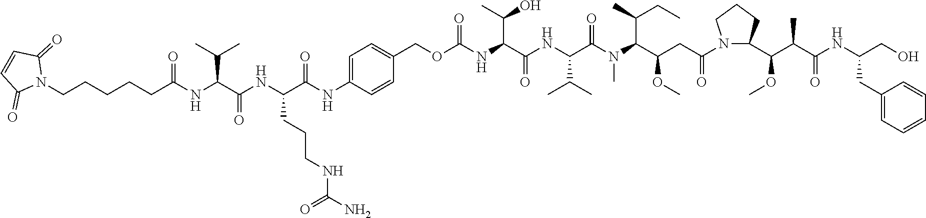 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](NC(=O)OCc1ccc(NC(=O)[C@H](CCCNC(N)=O)NC(=O)[C@@H](NC(=O)CCCCCN2C(=O)C=CC2=O)C(C)C)cc1)[C@@H](C)O)C(C)C]

Compound 2.11 was synthesized using method A. 64% yield. Analytical UPLC-MS: t_{R}=1.77 min, m/z (ES+) 1304.75 (M+H)⁺, found 1304.61.

### Example 65

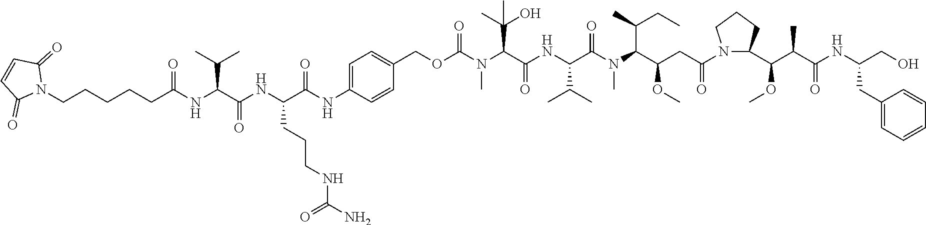 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)(C)O)C(C)C]

Compound 2.12 was synthesized using method A. 15% yield. Analytical UPLC-MS: t_{R}=1.92 min, m/z (ES+) 1332.78 (M+H)⁺, found 1333.51.

### Example 66

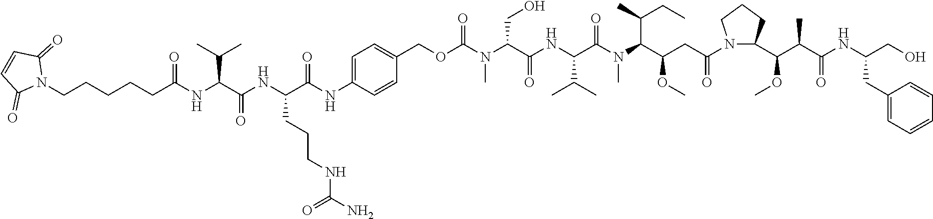 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](CO)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

Compound 2.13 was synthesized using method A. 57% yield. Analytical UPLC-MS: t_{R}=1.68 min, m/z (ES+) 1304.75 (M+H)+, found 1305.43.

### Example 67

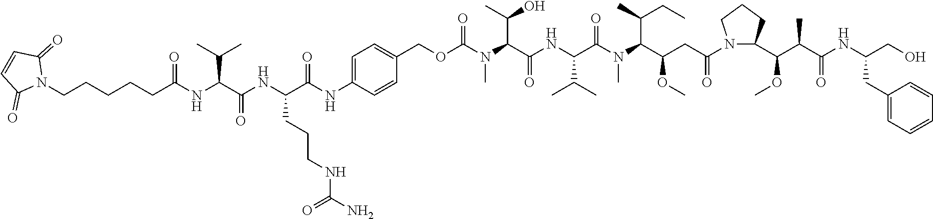 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H]([C@@H](C)O)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

Compound 2.14 was synthesized using method A. 47% yield. Analytical UPLC-MS: t_{R}=1.72 min, m/z (ES+) 1318.77 (M+H)⁺, found 1319.31.

### Example 68

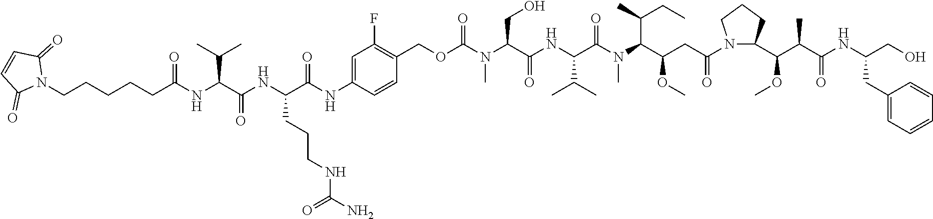 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)N(C)C(=O)OCc1ccc(NC(=O)[C@H](CCCNC(N)=O)NC(=O)[C@@H](NC(=O)CCCCCN2C(=O)C=CC2=O)C(C)C)cc1F)C(C)C]

Compound 2.19 was synthesized using method A. 80% yield. Analytical UPLC-MS: t_{R}=1.75 min, m/z (ES+) 1322.74 (M+H)⁺, found 1323.30.

### Example 69

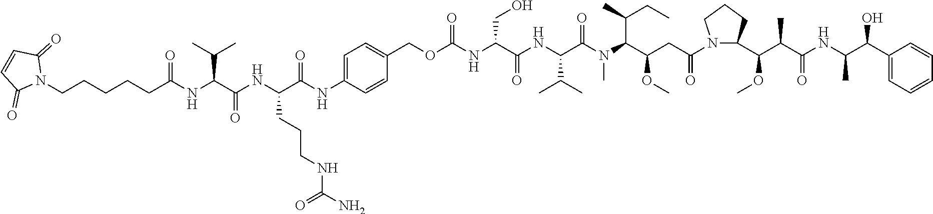 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](CO)NC(=O)OCc1ccc(NC(=O)[C@H](CCCNC(N)=O)NC(=O)[C@@H](NC(=O)CCCCCN2C(=O)C=CC2=O)C(C)C)cc1)C(C)C]

Compound 2.20 was synthesized using method A. 22% yield. Analytical UPLC-MS: t_{R}=1.67 min, m/z (ES+) 1291.58 (M+H)⁺, found 1291.42.

### Example 70

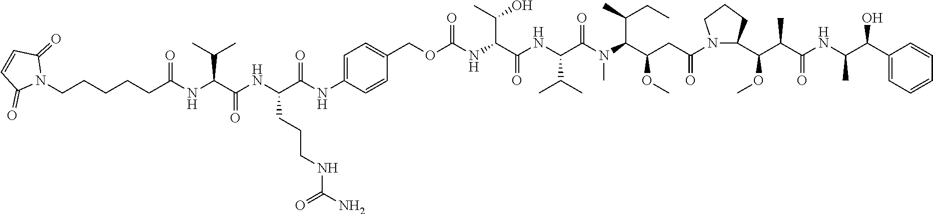 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](NC(=O)OCc1ccc(NC(=O)[C@H](CCCNC(N)=O)NC(=O)[C@@H](NC(=O)CCCCCN2C(=O)C=CC2=O)C(C)C)cc1)[C@H](C)O)C(C)C]

Compound 2.3 was synthesized using method A. Analytical UPLC-MS: t_{R}=1.71 min, m/z (ES+) 1305.60 (M+H)⁺, found 1304.91.

### Example 71

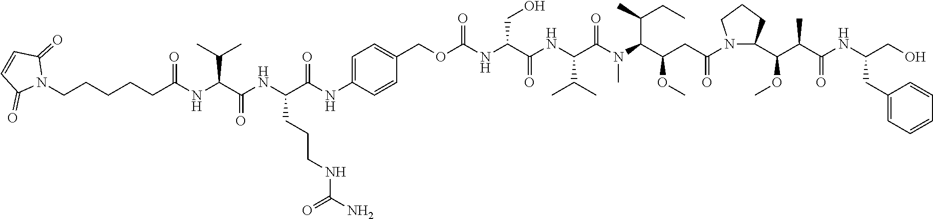 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H](CO)NC(=O)OCc1ccc(NC(=O)[C@H](CCCNC(N)=O)NC(=O)[C@@H](NC(=O)CCCCCN2C(=O)C=CC2=O)C(C)C)cc1)C(C)C]

Compound 2.29 was synthesized using method A.

### Example 72

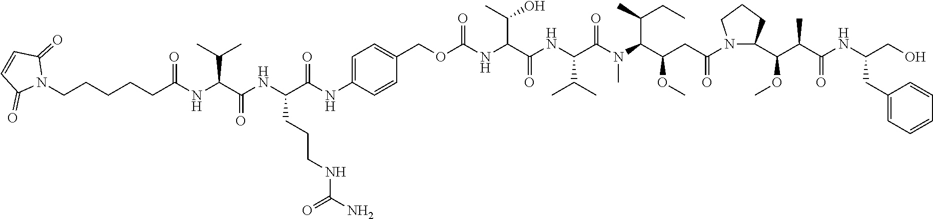 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)C(NC(=O)OCc1ccc(NC(=O)[C@H](CCCNC(N)=O)NC(=O)[C@@H](NC(=O)CCCCCN2C(=O)C=CC2=O)C(C)C)cc1)[C@H](C)O)C(C)C]

Compound 2.21 was synthesized using method A.

### Example 73

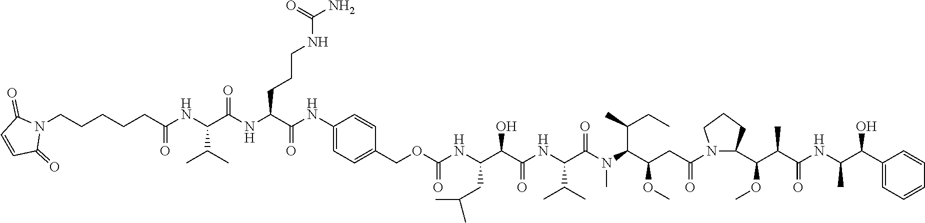 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](O)[C@H](CC(C)C)NC(=O)OCc1ccc(NC(=O)[C@H](CCCNC(N)=O)NC(=O)[C@@H](NC(=O)CCCCCN2C(=O)C=CC2=O)C(C)C)cc1)C(C)C]

Compound 2.22 was synthesized using method A. 27% yield. Analytical UPLC-MS: t_{R}=1.87 min, m/z (ES+) 1346.80 (M+H)⁺, found 1347.50.

### Example 74

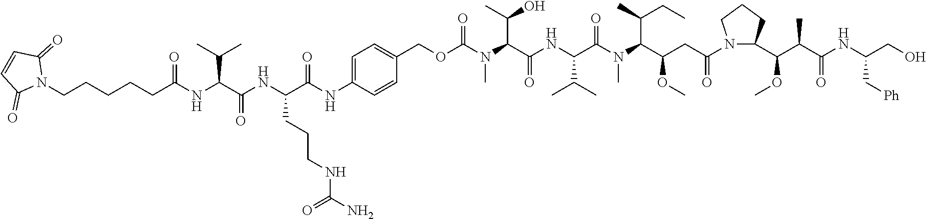 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H]([C@@H](C)O)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

Compound 2.14 was synthesized using method A. Analytical UPLC-MS: t_{R}=1.57 min, m/z (ES+) 1332.77 (M+H)+, found 1332.81.

### Example 75

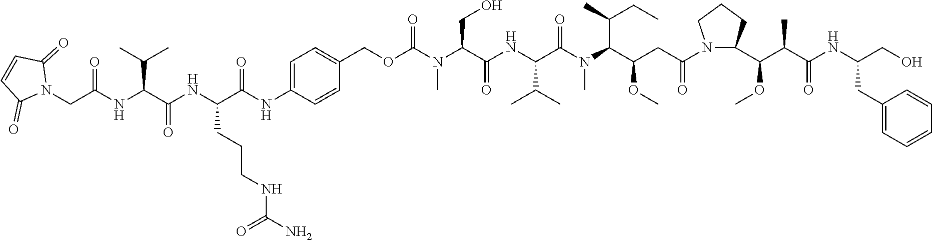 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)N(C)C(=O)OCc1ccc(NC(=O)[C@H](CCCNC(N)=O)NC(=O)[C@@H](NC(=O)CN2C(=O)C=CC2=O)C(C)C)cc1)C(C)C]

Compound 2.4 was synthesized using method A. Analytical UPLC-MS: t_{R}=1.64 min, m/z (ES+) 1248.69 (M+H)+, found 1249.42.

### Example 76

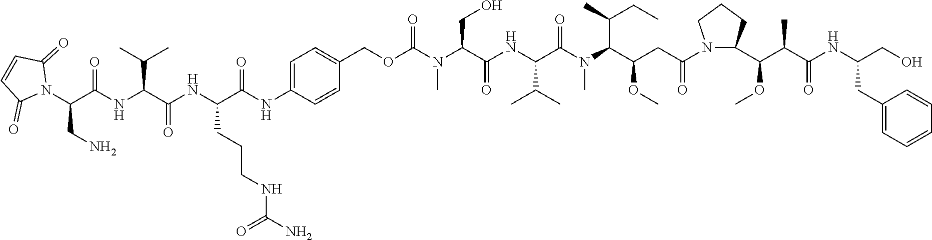 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)N(C)C(=O)OCc1ccc(NC(=O)[C@H](CCCNC(N)=O)NC(=O)[C@@H](NC(=O)[C@@H](CN)N2C(=O)C=CC2=O)C(C)C)cc1)C(C)C]

Compound 2.5 was synthesized using method A. Analytical UPLC-MS: t_{R}=1.85 min, m/z (ES+) 1277.71 (M+H)⁺, found 1278.21.

### Example 77

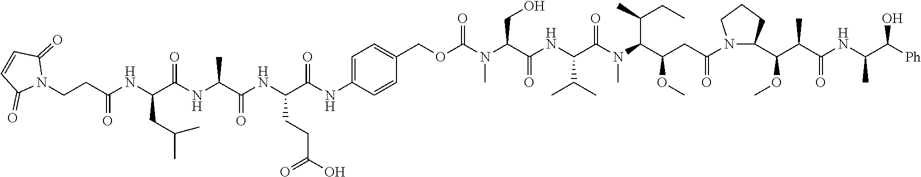 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](CO)N(C)C(=O)OCc1ccc(NC(=O)[C@H](CCC(=O)O)NC(=O)[C@H](C)NC(=O)[C@@H](CC(C)C)NC(=O)CCN2C(=O)C=CC2=O)cc1)C(C)C]

Compound 2.6 was synthesized using method B. Analytical UPLC-MS: t_{R}=1.69 min, m/z (ES+) 1319.71 (M+H)⁺, found 1319.75.

### Example 78

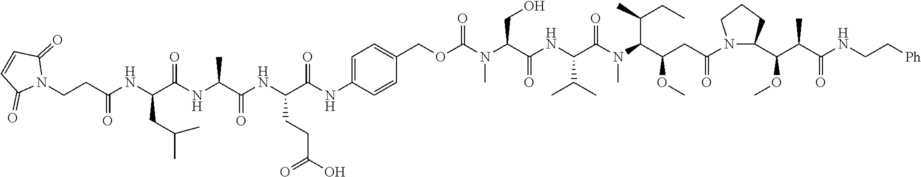 [SMILES: CC[C@H](C)[C@@H]([C@@H](CC(=O)N1CCC[C@H]1[C@H](OC)[C@@H](C)C(=O)NCCc1ccccc1)OC)N(C)C(=O)[C@@H](NC(=O)[C@H](CO)N(C)C(=O)OCc1ccc(NC(=O)[C@H](CCC(=O)O)NC(=O)[C@H](C)NC(=O)[C@@H](CC(C)C)NC(=O)CCN2C(=O)C=CC2=O)cc1)C(C)C]

Compound 2.16 was synthesized using method B. Analytical UPLC-MS: t_{R}=1.71 min, m/z (ES+) 1289.70 (M+H)⁺, found 1289.83.

### Example 79

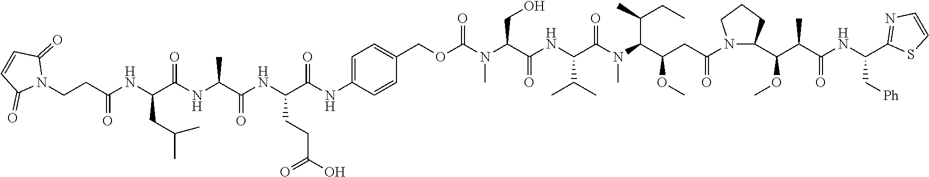 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H](CO)N(C)C(=O)OCc1ccc(NC(=O)[C@H](CCC(=O)O)NC(=O)[C@H](C)NC(=O)[C@@H](CC(C)C)NC(=O)CCN2C(=O)C=CC2=O)cc1)C(C)C]

Compound 2.17 was synthesized using method B. Analytical UPLC-MS: t_{R}=1.75 min m/z (ES+) 1372.69 (M+H)+, found 1372.75.

### Example 80

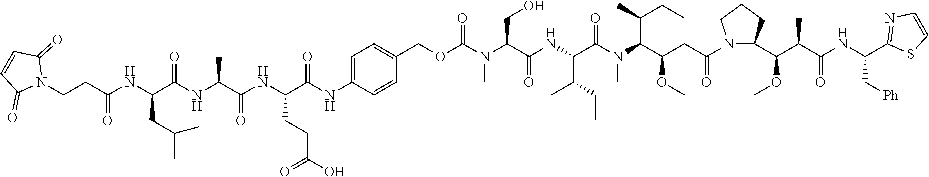 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H](CO)N(C)C(=O)OCc1ccc(NC(=O)[C@H](CCC(=O)O)NC(=O)[C@H](C)NC(=O)[C@@H](CC(C)C)NC(=O)CCN2C(=O)C=CC2=O)cc1)[C@@H](C)CC]

Compound 2.18 was synthesized using method B. Analytical UPLC-MS: t_{R}=1.81 min m/z (ES+) 1386.70 (M+H)+, found 1386.73.

### Example 81

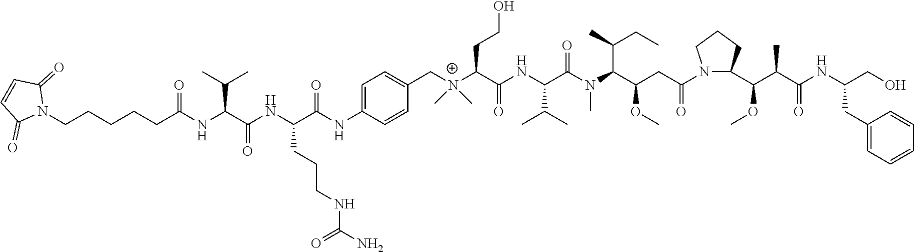 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CCO)[N+](C)(C)Cc1ccc(NC(=O)[C@H](CCCNC(N)=O)NC(=O)[C@@H](NC(=O)CCCCCN2C(=O)C=CC2=O)C(C)C)cc1)C(C)C]

Compound 2.23 was synthesized using method C. Analytical UPLC-MS: t_{R}=1.52 min, m/z (ES+) 1288.79 (M)⁺, found 1288.88.

### Example 82

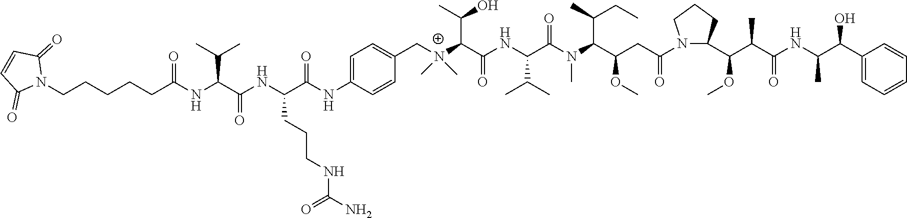 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H]([C@@H](C)O)[N+](C)(C)Cc1ccc(NC(=O)[C@H](CCCNC(N)=O)NC(=O)[C@@H](NC(=O)CCCCCN2C(=O)C=CC2=O)C(C)C)cc1)C(C)C]

Compound 2.24 was synthesized using method C. Analytical UPLC-MS: t_{R}=1.39 min, m/z (ES+) 1288.79 (M)⁺, found 1288.82.

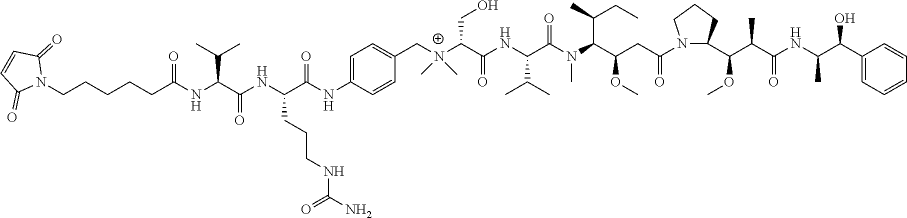 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](CO)[N+](C)(C)Cc1ccc(NC(=O)[C@H](CCCNC(N)=O)NC(=O)[C@@H](NC(=O)CCCCCN2C(=O)C=CC2=O)C(C)C)cc1)C(C)C]

Compound 2.25 was synthesized using method C. Analytical UPLC-MS: t_{R}=1.51 min, m/z (ES+) 1274.78 (M)⁺, found 1274.88.

### Example 84

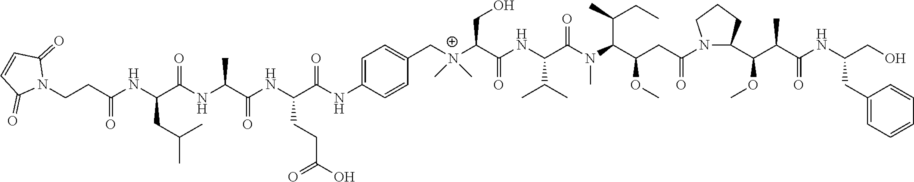 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)[N+](C)(C)Cc1ccc(NC(=O)[C@H](CCC(=O)O)NC(=O)[C@H](C)NC(=O)[C@@H](CC(C)C)NC(=O)CCN2C(=O)C=CC2=O)cc1)C(C)C]

Compound 2.26 was synthesized using method C. Analytical UPLC-MS: t_{R}=1.60 m, m/z (ES+) 1289.74 (M)⁺, found 1289.92.

### Example 85

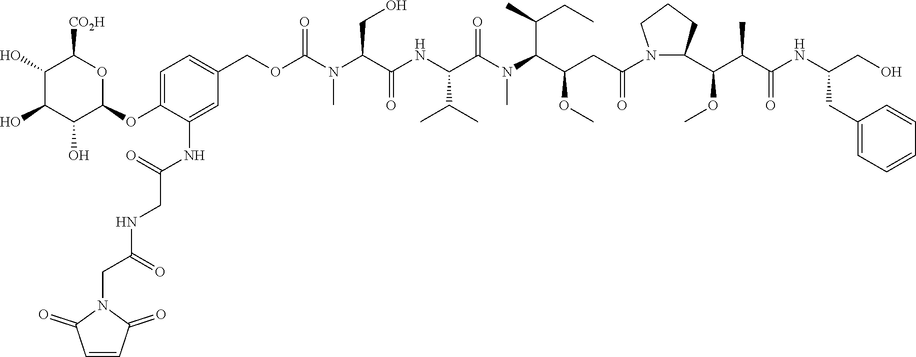 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)N(C)C(=O)OCc1ccc(O[C@@H]2O[C@H](C(=O)O)[C@@H](O)[C@H](O)[C@H]2O)c(NC(=O)CNC(=O)CN2C(=O)C=CC2=O)c1)C(C)C]

Modified-compound 2.7 synthesized according to a known procedure (Burke, P. J. et al. Mol. Cancer Ther. 2017, 16, 116-123).

Analytical UPLC-MS: t_{R}=1.68 min, m/z (ES+) 1241.58 (M+H)+, found 1241.25.

### Example 86. Measurement of LogD

The LogD (distribution coefficient) of the compounds was measured using a miniaturized 1-octanol/pH 7.4 buffer shake flask method followed by LC/MS/MS analysis. Values were determined by measuring the compound partitioned in the organic and buffered layers. Results are shown in Table 4.

### Example 87. In Vitro Cytotoxicity

The cytotoxicity of free drugs and drug conjugates were measured by a cell proliferation assay employing the protocol described in Promega Corp. Technical Bulletin TB288; and Mendoza et al., 2002, Cancer Res. 62:5485-5488), the methods of which are specifically incorporated by reference herein. Briefly, an aliquot of 40 μl of cell culture containing about 400 cells in medium is deposited in each well of a 384-well, opaque-walled plate. A 10 μL aliquot of free drug or drug conjugate is added to the experimental wells and incubated for 96 h and are then equilibrated to room temperature for approximately 30 minutes whereupon a volume of CellTiter-Glo™ reagent equal to the volume of cell culture medium present in each well is added. The contents are mixed for 2 minutes on an orbital shaker to induce cell lysis and the plate is incubated at room temperature for 10 minutes to stabilize the luminescence signal for recordation. Results are shown in Table 4.

TABLE 4A
Auristatin free drug LogD values and in vitro IC₅₀ (nM) values on
cancer cell lines.

| | Table 1 | | | | |
|---|---|---|---|---|---|
| Free drug | compound | DEL | Karpas299 | L540cy | |
| Example # | number | ALCL | NHL | HL | LogD |
| 26 | 1.72 | 4.2 | 5 | 20 | |
| 32 | 1.56 | 64 | 56 | 920 | 0.3 |
| 12 | 1.9 | 1.1 | 0.3 | 4.8 | 1.2 |
| 28 | 1.23 | 7.8 | 9.4 | 28 | 2.0 |
| 36 | 1.50 | 6.7 | 27 | 197 | |
| 23 | 1.16 | 17 | 66 | 662 | |
| 40 | 1.46 | 12.3 | 60 | 442 | |
| 24 | 1.18 | 13 | 43 | 621 | |
| 41 | 1.47 | 15 | 62 | 457 | |
| 25 | 1.20 | 18.9 | 62 | 457 | |
| 42 | 1.48 | 16 | 64 | 481 | |
| 13 | 1.12 | 1.2 | 2.2 | 5 | 1.4 |
| 43 | 1.60 | 3.2 | 5.9 | 19 | |
| 17 | 1.29 | 2.5 | 7.6 | 16.2 | 1.1 |
| 18 | 1.30 | 0.6 | 2.3 | 16 | 1.9 |
| 19 | 1.32 | 0.4 | 0.5 | 2.5 | 2.4 |
| 37 | 1.53 | >1000 | >1000 | >1000 | |
| 38 | 1.54 | 148 | >1000 | 512 | |
| 39 | 1.55 | 512 | >1000 | >1000 | |
| 8 | 1.1 | 0.7 | 1.4 | 6.6 | 1.6 |
| 45 | 1.52 | 8.5 | 40.8 | 272 | 1.5 |
| 14 | 1.10 | 3.5 | 7.4 | 10 | 1.1 |
| 20 | 1.33 | 0.3 | 1 | 4.7 | 1.9 |
| 15 | 1.13 | 0.7 | 2.3 | 3.9 | |
| 22 | 1.58 | 5.8 | 13 | 16 | |
| 33 | 1.59 | 1.8 | 1.7 | 6.4 | 3.2 |
| 29 | 1.21 | 12.7 | 13.7 | 58 | 2.0 |
| 30 | 1.24 | 396 | 481 | >1000 | 1.2 |
| 31 | 1.25 | 2.1 | 1.7 | 4.7 | 3.4 |
| 27 | 1.22 | 1.9 | 2.3 | 13 | |
| 9 | 1.3 | 0.8 | 0.9 | 4 | |
| 10 | 1.4 | 0.4 | 0.5 | 3 | |
| 11 | 1.2 | 1 | 1 | 6 | |
| 46 | 1.61 | 141 | 264 | >1000 | |
| 47 | 1.28 | >1000 | >1000 | >1000 | |
| 48 | 1.62 | 41 | 110 | >1000 | |
| 49 | 1.41 | 32 | 35 | 76 | |
| 50 | 1.64 | >1000 | >1000 | >1000 | |
| 51 | 1.65 | 93 | 224 | >1000 | |
| 52 | 1.66 | 43 | 58 | 438 | |
| 53 | 1.67 | >1000 | >1000 | >1000 | |
| 54 | 1.68 | 7 | 6 | 274 | |
| 55 | 1.69 | 0.8 | 1 | 16 | |
| 56 | 1.70 | 1 | 0.9 | 24 | |
| 57 | 1.71 | 4 | 3 | 49 | |
| MMAE | — | 0.05 | 0.1 | 0.1 | 1.9 |
| MMAF | — | 44 | 41 | 47 | 0.03 |

TABLE 4B
Auristatin free drug in vitro IC₅₀ (nM) values on cancer cell lines.

| | Table 1 | | | |
|---|---|---|---|---|
| Free Drug | compound | DEL | Karpas299 | L540cy |
| Example # | number | ALCL | NHL | HL |
| Comparative | | 209 | 253 | >1000 |
| Example A | | | | |
| 12 | 1.9 | 1.1 | 0.3 | 4.8 |
| MMAE | | 0.05 | 0.1 | 0.1 |
| MMAF | | 44 | 41 | 47 |
| Comparative | | 342 | 218 | 284 |
| Example C | | | | |
| 40 | 1.46 | 12.3 | 60 | 442 |

### Example 88. Conjugation of Drug-Linker Compounds to Antibodies to Form Ligand-Drug Conjugate Compounds

Antibodies were partially reduced using the appropriate equivalents of TCEP according to the procedure, which is specifically incorporated by reference herein, of US 2005/0238649. Briefly, the antibody in phosphate buffered saline with 2 mM EDTA, pH 7.4, was treated with 2.1 eq. TCEP and then incubated at 37° C. for about 45 minutes. The thiol/Ab value was checked by reacting the reduced antibody with MC-VC-MMAE and using hydrophobic interaction chromatography to determine the loading.

The auristatin drug linker compounds were conjugated to the partially reduced antibody using the method, which is specifically incorporated by reference herein, of US 2005/0238649. Briefly, Drug-Linker compound (50% excess) in DMSO, was added to the reduced antibody in PBS with EDTA along with additional DMSO for a total reaction co-solvent of 10-20%. After 30 minutes at ambient temperature, an excess of QuadraSil MP™ was added to the mixture to quench all unreacted maleimide groups. The resulting Ligand-Drug Conjugate compound was then purified, and buffer exchanged by desalting using Sephadex G25 resin into pH 6.5 histidine buffer and kept at −80° C. until further use. The protein concentration of the resulting ADC composition was determined at 280 nm. The drug-antibody ratio (DAR) was confirmed by comparing predicted and observed changes in the molecular weights of heavy and light chains using PLRP-MS analysis and confirming absence of underloaded species.

### Example 89. In Vivo Cytotoxicity of Ligand-Drug Conjugate Compounds

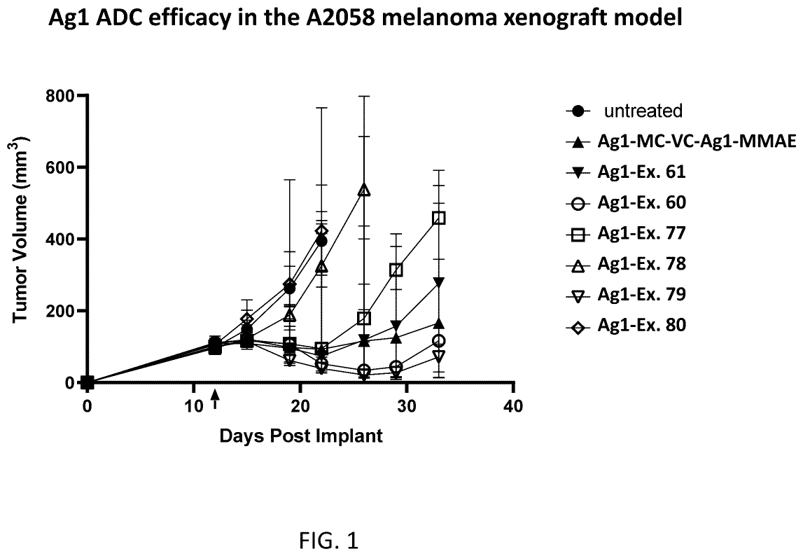

Cancer cells were implanted into mice. Upon tumor engraftment, mice were randomized to study groups (n=5) once the average tumor volume reached approximately 100 mm³. ADC prepared from reduced antibody and compound was administered via an intraperitoneal or intravenous injection. Tumor volumes are calculated using the formula (0.5×L×W2) where L and W are the longer and shorter of two bidirectional measurements. Complete response (CR) is defined here as absence of measurable mass at tumor site during an experiment. Results are shown in FIGS. 1 to 5 and in Table 5 for Ligand-Drug Conjugate compounds with an average drug loading of 4 Drug-Linker moieties per antibody.

FIG. 1 shows the efficacy of a 6 mg/kg dose of a variety of Ag1 ADCs as measured in the A2058 melanoma xenograft model. The data show that Ag1 ADCs comprising hydrophilic auristatin Drug Units have efficacy comparable to ADCs comprising MMAE.

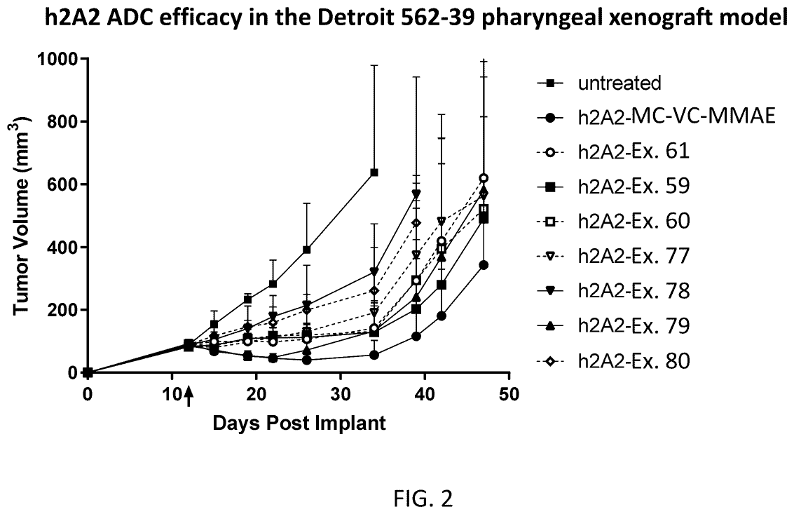

FIG. 2 shows the efficacy of a 3 mg/kg dose of a variety of h2A2 ADCs as measured in the Detroit 562-39 pharyngeal xenograft model. The data show that h2A2 ADCs comprising hydrophilic auristatin Drug Units have efficacy comparable to ADCs comprising MMAE.

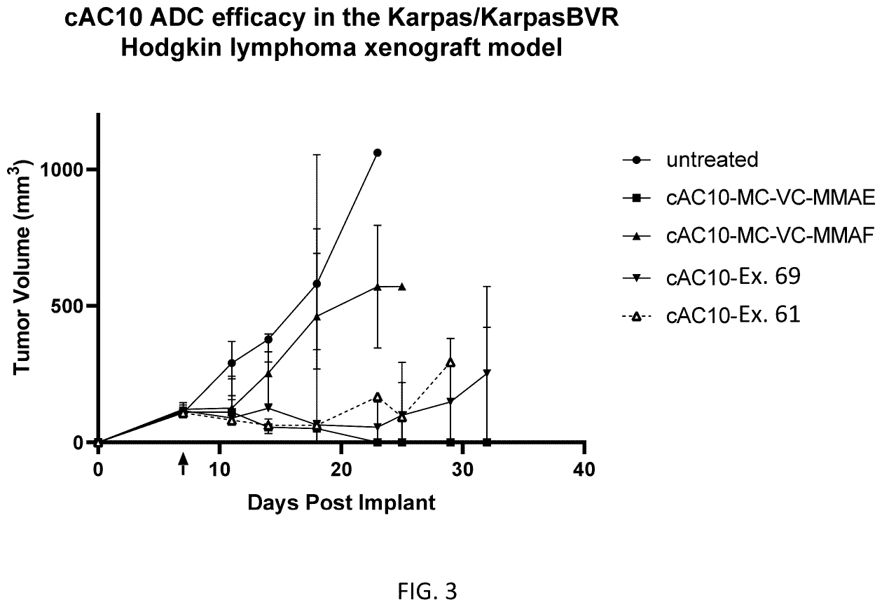

FIG. 3 shows the efficacy of a 3 mg/kg dose of a variety of cAC10 ADCs as measured in the Karpas/KarpasBVR admixed Hodgkin lymphoma xenograft model. The data show that cAC10 ADCs with hydrophilic auristatin Drug Units have bystander activity between that of MC-VC-MMAE and MC-VC-MMAF.

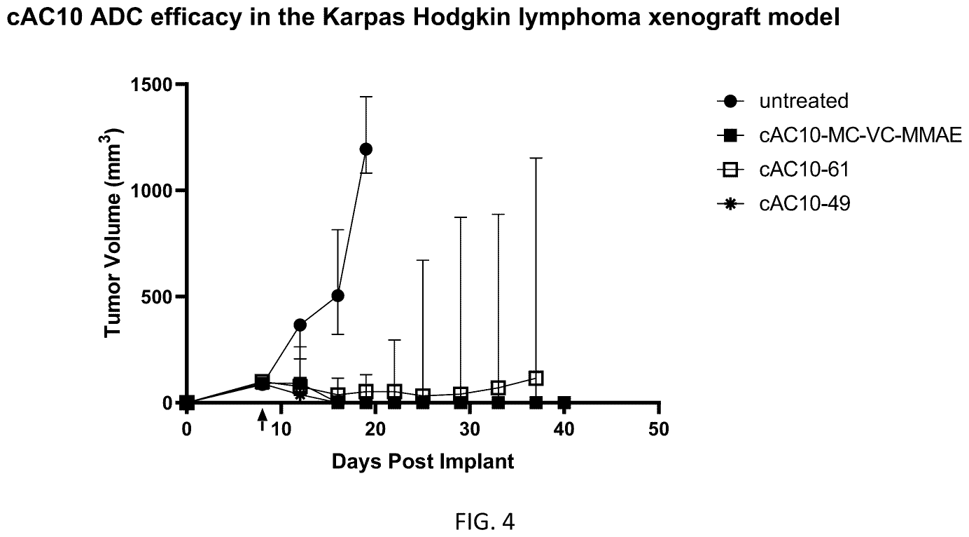

FIG. 4 shows the efficacy of a 1 mg/kg dose of a variety of cAC10 ADCs in the Karpas Hodgkin lymphoma xenograft model. The data show that cAC10 ADCs comprising hydrophilic auristatin Drug Units have efficacy comparable to ADCs comprising MMAE.

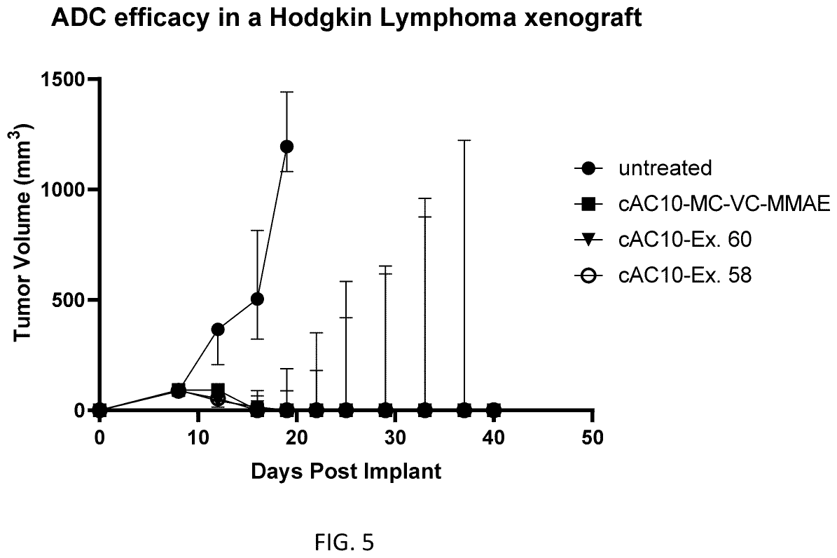

FIG. 5 shows the efficacy of a 1 mg/kg dose of a variety of cAC10 ADCs in the Karpas Hodgkin xenograft model. The data show that cAC10 ADCs comprising hydrophilic auristatin Drug Units have efficacy comparable to ADCs comprising MMAE.

### Example 90. Toxicity Determinations of Ligand-Drug Conjugate Compounds

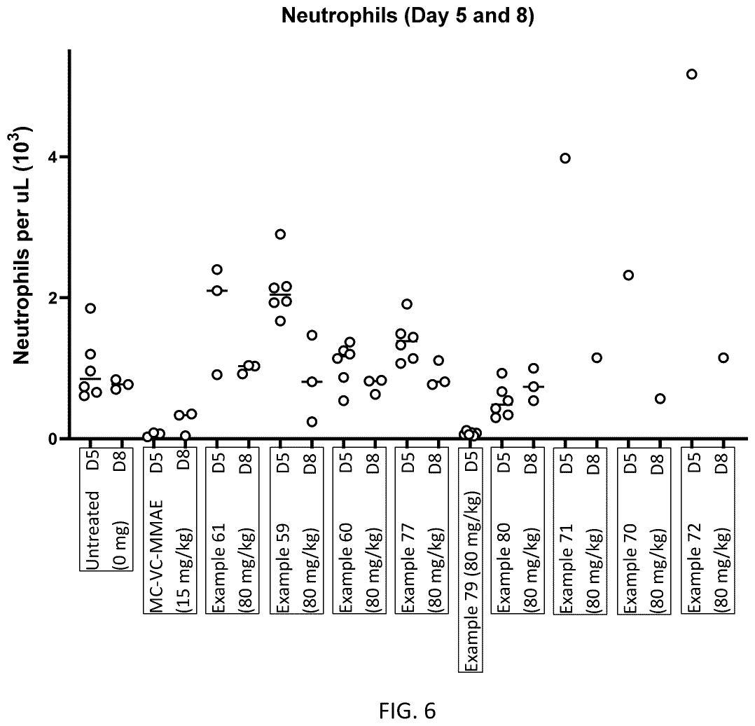

In vivo rat safety studies were done in naive female Sprague Dawley rats (Envigo). Studies had 3 animals/group/timepoint and included a vehicle control group (1×PBS pH 7.4). Animals were administered a single dose of 15 or 80 mg/kg of an h00 ADC (DAR=4) via intravenous injection. Blood was drawn at days 5 and day 8. Blood was sampled under isoflurane anesthesia via the jugular vein for periodic hematology analysis on the Sysmex XT-2000iV. At necropsy, blood was collected from the caudal vena cava for hematology and clinical chemistry panels. Clinical chemistry was analyzed on a Beckman Coulter AU680. Results are shown in FIGS. 6 to 9.

FIG. 6 demonstrates that neutrophil counts are higher at on Day 5 and recovery of neutrophil counts on Day 8 are higher after administering Ligand-Drug Conjugate compounds comprising hydrophilic auristatin Drug Units when compared to MMAE containing conjugates.

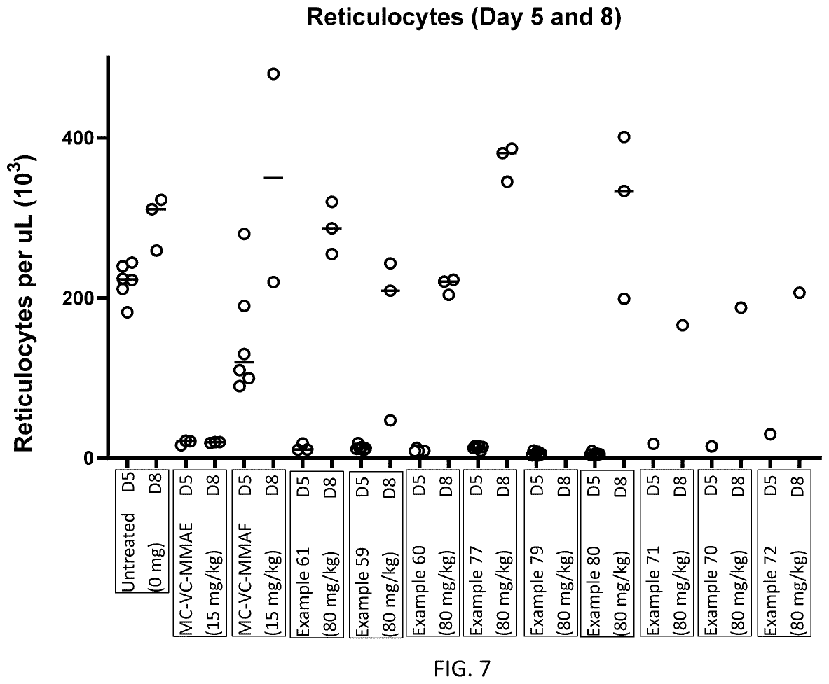

FIG. 7 demonstrates that recovery of reticulocytes is greatly improved after administering Ligand-Drug Conjugate compounds comprising hydrophilic auristatin Drug Units when compared to MMAE containing conjugates.

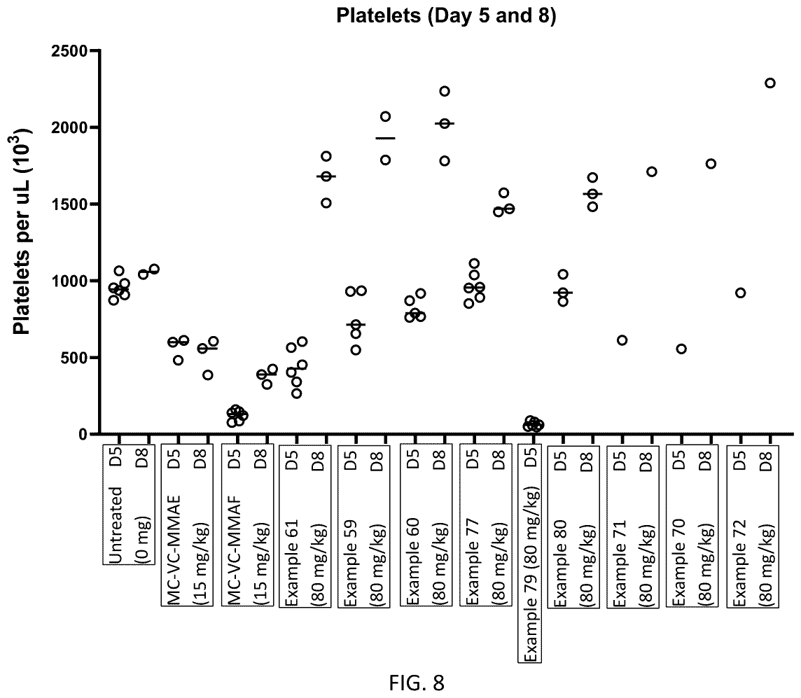

FIG. 8 shows that administering Ligand-Drug Conjugate compounds with hydrophilic auristatin Drug Units does not impact the platelet count as heavily as administering an MMAF containing conjugate. Recovery of platelets is also better after administering Ligand-Drug Conjugate compounds comprising hydrophilic auristatin Drug Units when compared to MMAE and MMAF containing conjugates.

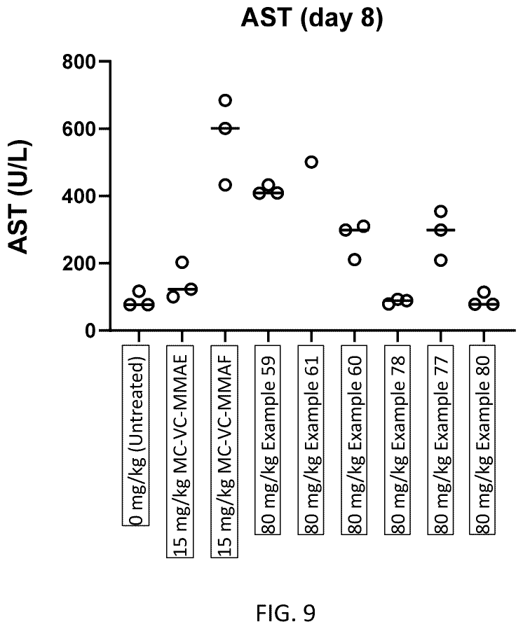

FIG. 9 shows that aspartate transaminase (AST) levels indicating liver damage are lower 8 days after administering 80 mg/kg of Ligand-Drug Conjugate compounds comprising hydrophilic auristatin Drug Units when compared to after administering 15 mg/kg of an MMAF containing conjugates. The AST levels 8 days following administration are comparable for an 80 mg/kg dose of some Ligand-Drug Conjugate compounds comprising hydrophilic auristatin Drug Units when compared to a 15 mg/kg dose of an MMAE conjugate.

TABLE 5A
In vitro IC₅₀ (ng/mL) values of auristatin anti-CD30 ADCs on CD30-
expressing cell lines using cAC10 ADCs with an average drug loading
of about 4 of selected Drug-Linker compounds.

| Drug-Linker | Table 2 | | | |
|---|---|---|---|---|
| moiety Example | compound | DEL | Karpas299 | L540cy |
| # | number | ALCL | NHL | HL |
| 69 | 2.20 | 0.01 | 0.2 | NA |
| 71 | 2.29 | NA | 7 | 9 |
| 72 | 2.21 | NA | 7 | 15 |
| 61 | 2.2 | 0.01 | 0.6 | 6.3 |
| 59 | 2.6 | 0.1 | 1.2 | 9.3 |
| 63 | 2.10 | 12 | 290 | >1000 |
| 62 | 2.9 | 3.8 | >1000 | >1000 |
| 64 | 2.11 | 22 | >1000 | >1000 |
| 76 | 2.5 | NA | NA | NA |
| 65 | 2.12 | 0.04 | 1.2 | 3.8 |
| 85 | 2.7 | 0.01 | 0.5 | 3.6 |
| 75 | 2.4 | 0.01 | 0.2 | 3.2 |
| 60 | 2.1 | 0.09 | 0.4 | 4.3 |
| 66 | 2.13 | 0.3 | 5.1 | 4.9 |
| 67 | 2.14 | 1.7 | 29 | 33 |
| 68 | 2.19 | 1 | 15 | 35 |
| 77 | 2.6 | 0.4 | 5.6 | 8.2 |
| 78 | 2.16 | 1.3 | 58 | >1000 |
| 79 | 2.17 | 1.5 | 0.9 | 4.7 |
| 80 | 2.18 | 1.4 | >1000 | >1000 |
| 70 | 2.3 | 0.03 | 1 | 1.9 |
| 84 | 2.26 | 0.01 | 0.1 | 13 |
| 73 | 2.22 | >1000 | >1000 | >1000 |
| 81 | 2.23 | 0.1 | 0.1 | 1 |
| 82 | 2.24 | 0.2 | 0.3 | 4 |
| 83 | 2.25 | 0.1 | 0.1 | 4 |
| MC-VC-MMAE | — | 0.4 | 0.6 | 2 |
| MC-VC-MMAF | — | 0.1 | 1 | 3 |

TABLE 5B
In vitro IC₅₀ (ng/mL) values of auristatin anti-CD30 ADCs on CD30-
expressing cell lines using cAC10 ADCs with an average drug loading
of about 4 of selected Drug-Linker compounds.

| Drug-Linker | Table 2 | | | |
|---|---|---|---|---|
| moiety | compound | DEL | Karpas299 | L540cy |
| Example # | number | ALCL | NHL | HL |
| Comparative | — | 0.01 | 0.01 | 0.1 |
| Example B | | | | |
| 61 | 2.2 | 0.01 | 0.06 | 6.3 |
| MC-VC-MMAE | — | 0.4 | 0.6 | 2 |
| MC-VC-MMAF | — | 0.1 | 1 | 3 |

TABLE 5C
In vitro IC₅₀ (ng/mL) values of auristatin anti-CD30 ADCs on CD30-
expressing cell lines using cAC10 ADCs with an average drug loading
of about 4 of selected Drug-Linker compounds.

| Drug-Linker | Table 2 | | | |
|---|---|---|---|---|
| moiety | compound | DEL | Karpas299 | L540cy |
| Example # | number | ALCL | NHL | HL |
| 61 | 2.2 | 0.01 | 0.6 | 6.3 |
| 75 | 2.4 | 0.01 | 0.2 | 3.2 |
| 76 | 2.5 | NA | NA | NA |
| 85 | 2.7 | 0.01 | 0.5 | 3.6 |
| 59 | 2.6 | 0.1 | 1.2 | 9.3 |
| 60 | 2.1 | 0.09 | 0.4 | 4.3 |
| 84 | 2.26 | 0.01 | 0.1 | 13 |
| MC-VC-MMAE | — | 0.4 | 0.6 | 2 |
| MC-VC-MMAF | — | 0.1 | 1 | 3 |

TABLE 5D
In vitro IC₅₀ (ng/mL) values and tolerability of auristatin anti-CD30 ADCs
on CD30-expressing cell lines using cAC10 ADCs with an average
drug loading of about 4 of selected Drug-Linker compounds.

| | | | | | Tolerated |
|---|---|---|---|---|---|
| Drug-Linker | Table 2 | | | | at 80 |
| moiety | compund | DEL | Karpas299 | L540cy | mg/kg in |
| Example # | number | ALCL | NHL | HL | rats |
| 61 | 2.2 | 0.01 | 0.6 | 6.3 | Yes |
| 78 | 2.16 | 1.3 | 58 | >1000 | Yes |
| 79 | 2.17 | 1.5 | 0.9 | 4.7 | No |
| 80 | 2.18 | 1.4 | >1000 | >1000 | Yes |

### Example 91. Comparison of Compound Tolerability

Tolerability of an ADC comprising the compound of Example 8 of the present application was compared to tolerability of ADCs comprising the known compounds MMAE and MMAF. Without being bound by theory, the improvements in tolerability are believed to be related to reduced toxicities in the bone marrow and in the liver that are a result of the OH moieties on the compound of Example 8. See Table 6 below.

TABLE 6
Tolerability comparison of compound 1.1 to MMAE and MMAF

| | | | Avg IC50 of | Maximum tolerated | |
|---|---|---|---|---|---|
| | | Avg | corresponding | dose** (mg/kg) | |
| | | IC50 | dipeptide | Based on | Based on |
| | | Free | linked | reduced | elevated |
| | | drug\hat{ } | ADC\hat{ }* | Neutrophil | AST |
| Compound | LogD | (nM) | (ng/mL) | counts | level |
| MMAE | 1.9 | 0.2 | 1 | 15 | >15 |
| Example 8 | 1.6 | 2.9 | 1.5 | >80 | >80 |
| (compound 1.1) | | | | | |
| MMAF | 0.03 | 44 | 1.3 | >15 | 15 |

\hat{ }Average cytotoxicity of DEL, Karpas299, and L540cy cell lines
*anti-CD30 ADCs
**Dosed in Sprague-Dawley Rats

### Example 92. In Vitro Quantitative Flow Cytometry of Cancer Cell Lines

Cancer cells were counted and 100,000-200,000 cells were plated in 100 μL FACS buffer in 96-well U-bottom plates (Falcon). Cells were incubated with 10 ug/mL of non-binding and targeted (hCR011) human IgG for one hour at 4° C. Cells were washed 3× before anti-IgG FITC secondary antibody and calibration beads from Human IgG Calibrator Kit (BioCytex Ref #CP101) were added for 30 minutes at 4° C. Cells were washed 2× and run on an Attune cytometer. Data was analyzed using FlowJo software (BD). Target antigen (gpNMB) copy number was determined per manufacturer's instructions; briefly, the MFI values of calibration beads were used to determine the antibody binding capacity and the specific antibody binding capacity (SABC) was determined by subtracting the non-binding IgG MFI from the targeted (hCR011) MFI. The SABC numbers for hCR011 (i.e. gpNMB copy number) are summarized in Table 7.

TABLE 7
gpNMB (hCR011) copy numbers for a panel of cancer cell lines

| Cell line | SABC |
|---|---|
| WM2664 | 14612 |
| SKMEL5 | 5035 |
| HCC2218 | 13207 |
| SKBR3 | 2839 |
| SKMEL28 | 8671 |
| CALU1 | 229 |
| A2058 | 2,781 |

### Example 93: In Vitro Cytotoxicity of Auristatin ADCs in a Panel of Cancer Cell Lines

Cancer cells were counted and plated in 40 μL complete growth media in 384-well, white-walled tissue culture treated plates (Corning). Cell plates were incubated at 37° C. and with 5% CO₂ overnight to allow the cells to equilibrate. Stock solutions containing ADCs or free drugs were serially diluted in RPMI-1640+20% fetal bovine serum (FBS). 10 μL of each concentration were then added to each cell plate in duplicate. Cells were then incubated at 37° C. and with 5% CO₂ for 96 hours, upon which, the cell plates were removed from the incubator and allowed to cool to room temperature for 30 minutes prior to analysis. CellTiter-Glo® luminescent assay reagent (Promega Corporation, Madison, WI) was prepared according to Promega's protocol. 10 μL of CellTiter-Glo® were added to assay plates using a Formulatrix Tempest liquid handler (Formulatrix) and the plates were protected from light for 30 minutes at room temperature. The luminescence of the samples was measured using an EnVision Multimode plate reader (Perkin Elmer, Waltham, MA). Raw data were analyzed in Graphpad Prism (San Diego, CA) using a nonlinear, 4-parameter curve fit model [Y=Bottom+(Top-Bottom)/(1+10\hat{ }((LogEC50−X)*HillSlope))]. Results are reported as IC50 values, which are defined as the concentration of ADC or free drug required to reduce cell viability to 50%. The cytotoxic activity of auristatin free drugs and ADCs on cancer cell lines is summarized in Tables 8 and 9.

TABLE 8
Cytotoxicity of auristatin ADCs on a panel of cancer cell lines

| | IC50 (ng/ml) | | | | |
|---|---|---|---|---|---|
| | Non- | | | | |
| | binding- | Non- | hCR011- | | |
| | ex. 60 | binding- | ex. 60 | hCR011- | |
| | (compound | MC-VC- | (compound | MC-VC- | Target |
| Cell line | 3.1) | MMAE | 3.1) | MMAE | expression* |
| A2058 | >30K | >30K | 19859 | 24300 | + |
| CALU1 | >30K | >30K | >30K | >30K | − |
| HCC2218 | >30K | >30K | >30K | >30K | + |
| SKMEL5 | 22203 | 11022 | 1832 | 916 | + |
| WM2664 | >30K | >30K | 1566 | 4468 | + |
| SKBR3 | 21695 | 16218 | 5394 | 2690 | + |

*Cancer cell lines with >2000 surface copy number of target antigen (determined by quantitative flow cytometry) were considered positive (“+”).

TABLE 9
Cytotoxicity of auristatin free drugs and ADCs on a panel of cancer cell lines

| | IC50 (ng/mL) | | | | | | |
|---|---|---|---|---|---|---|---|
| | Non- | | | | | | |
| | binding- | Non- | | | IC50 (nM) | | |
| | Ex. 60 | binding- | | hCR011- | Ex. 8 | | |
| | (compound | MC-VC- | hCR011- | MC-VC- | (compound | | Target |
| Cell line | 3.1) | MMAE | Ex. 60 | MMAE | 1.1) | MMAE | expression* |
| A2058 | >30K | >30K | 12858 | 11118 | 2 | 0.3 | + |
| CALU1 | >30K | >30K | >30K | 14251 | 5 | 0.6 | − |
| SKMEL28 | >30K | >30K | 6913 | 14020 | 4 | 0.4 | + |
| SKMEL5 | 29560 | 13119 | 1513 | 668 | 2 | 0.2 | + |

*Cancer cell lines with >2000 surface copy number of target antigen (determined by quantitative flow cytometry) were considered positive (“+”).

### Example 94: In Vivo Antitumor Activity of Auristatin ADCs in WM2664 and SKMEL5 Xenograft Tumor Models

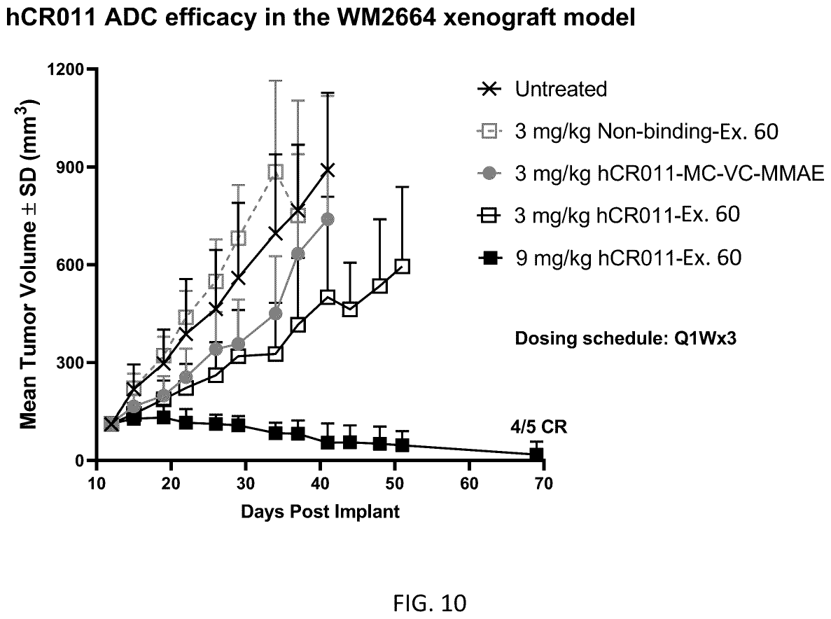

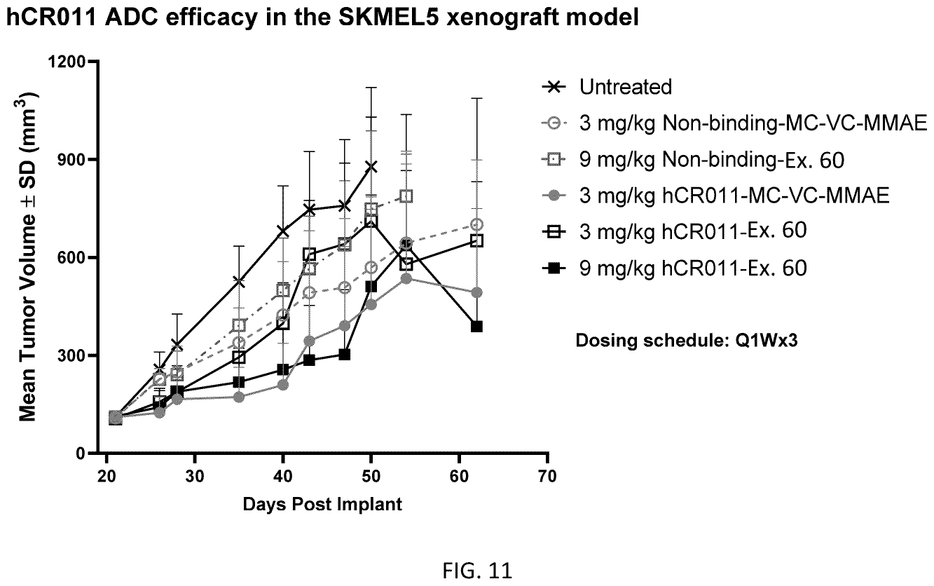

Cancer cells were implanted into nude mice. Upon tumor engraftment, mice were randomized to study groups (n=5) and when tumor volumes reached 100 mm³, the mice were dosed with ADCs by intravenous injection at the indicated dose Q1W×3 (3 weekly doses) and tumor volumes were monitored twice weekly. Tumor volumes are calculated using the formula (0.5×L×W2) where L and W are the longer and shorter of two bidirectional measurements. Complete response (CR) is defined here as absence of measurable mass at tumor site during an experiment. Results are shown in FIGS. 10-11 for Ligand-Drug Conjugate compounds with an average drug loading of 4 Drug-Linker moieties per antibody.

FIG. 10 shows the efficacy of a variety of hCR011 ADCs and a non-binding control ADC as measured in the WM2664 melanoma xenograft model. The data show that hCR011 ADCs comprising hydrophilic auristatin Ex. 60 dosed at 3 mg/kg (3 weekly doses) have efficacy comparable to ADCs comprising MMAE. When hCR011-Ex. 60 ADCs are dosed at 9 mg/kg (3 weekly doses), there are 4/5 complete responses.

FIG. 11 shows the efficacy of a variety of hCR011 ADCs and non-binding control ADCs as measured in the SKMEL5 melanoma xenograft model. The data show that hCR011 ADCs comprising hydrophilic auristatin Ex. 60 dosed at 9 mg/kg (3 weekly doses) have efficacy comparable to ADCs comprising MMAE dosed at 3 mg/kg (3 weekly doses).

### Example 95: Antitumor Activity of Auristatin ADCs in Patient-Derived Xenograft (PDX) Models of Melanoma and Non-Small Cell Lung Cancer (NSCLC

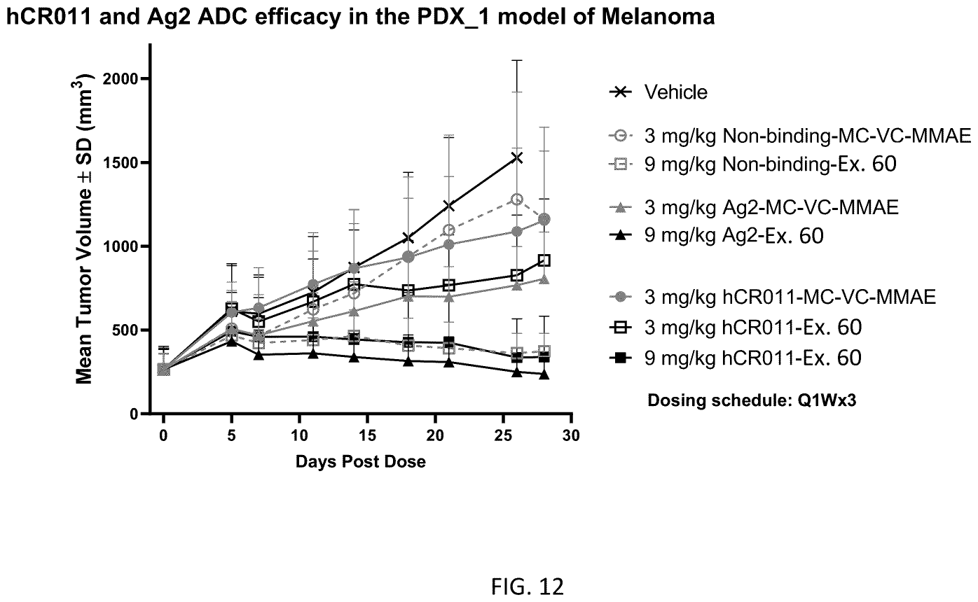

Cancer cells were implanted into Nude (Nude-Foxn1^{nu}) female mice. When tumors reached an average tumor volume of 150-300 mm³ animals were matched by tumor volume into treatment or control groups and dosed with ADCs by intravenous injection at the indicated dose Q1W×3 (3 weekly doses) and tumor volumes were monitored twice weekly. The study endpoint was when the mean tumor volume of the control group (uncensored) reached 1500 mm³. Complete response (CR) is defined here as absence of measurable mass at tumor site during an experiment. Results are shown in FIGS. 12-15 for Ligand-Drug Conjugate compounds with an average drug loading of 4 Drug-Linker moieties per antibody. Ag2 refers to an antibody targeting a ubiquitous and readily internalizable antigen on cancer cells.

FIG. 12 shows the efficacy of non-binding, Ag2, and hCR011 ADCs as measured in the PDX_1 melanoma xenograft model. The data show that hCR011 ADCs comprising hydrophilic auristatin Ex. 60 (compound 3.1 wherein L is hCR011) dosed at 3 mg/kg have efficacy comparable to ADCs comprising MMAE dosed at 3 mg/kg. Ag2 and hCR011 ADCs with Ex. 60 (compound 3.1 wherein L is hCR011) dosed at 9 mg/kg elicited more robust antitumor activity.

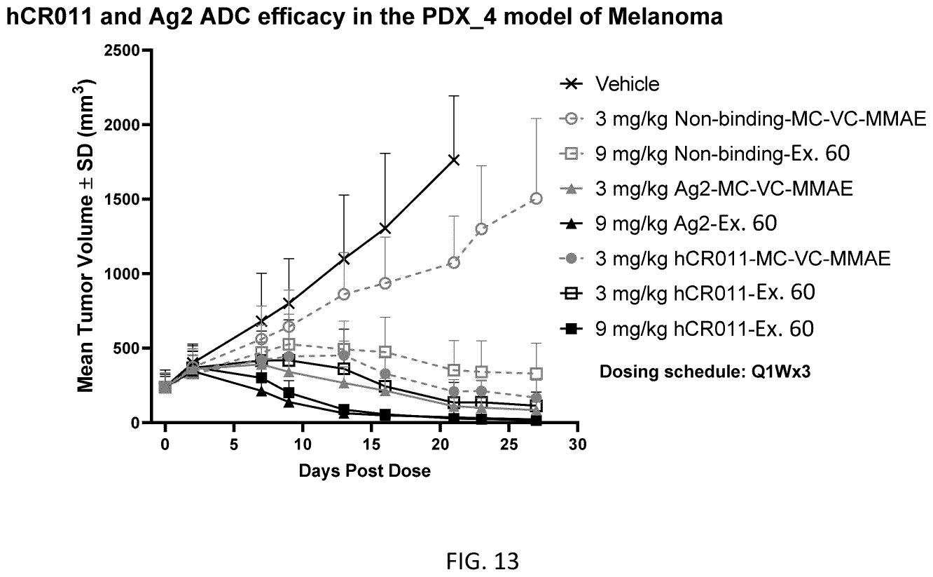

FIG. 13 shows the efficacy of non-binding, Ag2, and hCR011 ADCs as measured in the PDX_4 melanoma xenograft model. The data show that hCR011 ADCs comprising hydrophilic auristatin Ex. 60 (compound 3.1 wherein L is hCR011) dosed at 3 mg/kg have efficacy comparable to ADCs comprising MMAE dosed at 3 mg/kg. Ag2 and hCR011 ADCs with Ex. 60 (compound 3.1 wherein L is Ag2 or hCR011, respectively) dosed at 9 mg/kg elicited more robust antitumor activity, with hCR011-Ex. 60 ADCs leading to 2/5 complete responses.

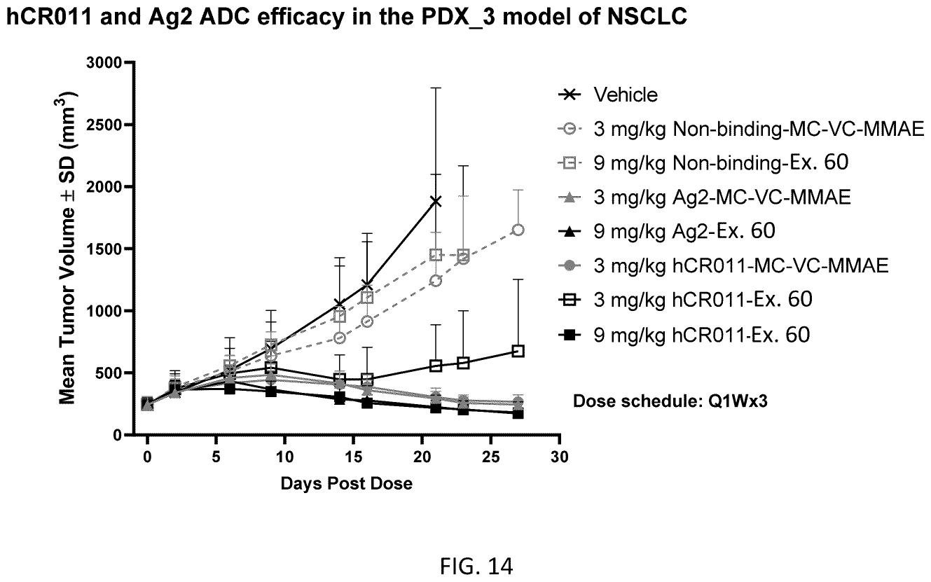

FIG. 14 shows the efficacy of non-binding, Ag2, and hCR011 ADCs as measured in the PDX_3 NSCLC xenograft model. The data show that Ag2 and hCR011 ADCs comprising hydrophilic auristatin Ex. 60 (compound 3.1 wherein L is Ag2 or hCR011, respectively) dosed at 3 and/or 9 mg/kg have efficacy comparable to ADCs comprising MMAE dosed at 3 mg/kg.

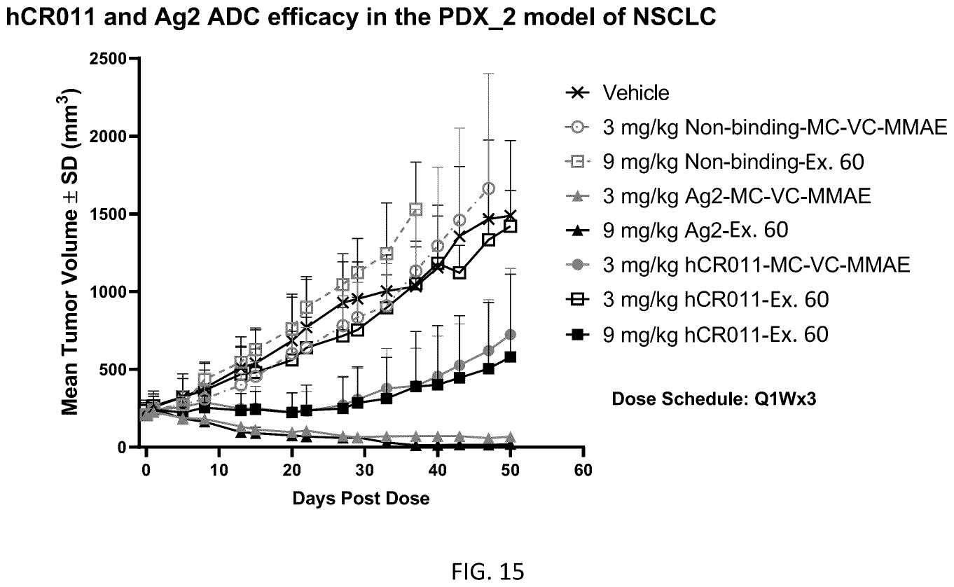

FIG. 15 shows the efficacy of non-binding, Ag2, and hCR011 ADCs as measured in the PDX_2 NSCLC xenograft model. The data show that Ag2 and hCR011 ADCs comprising hydrophilic auristatin Ex. 60 (compound 3.1 wherein L is Ag2 or hCR011, respectively) dosed as 9 mg/kg have comparable efficacy to ADCs comprising MMAE dosed at 3 mg/kg.

### Example 96: Efficacy Comparison of In Vivo Tumor Models

Average Slope or AUC.3 was used to compare the anti-tumor activity of 6 different xenograft models. Tumor growth was assessed by the metric area under the curve (AUC), “AUC.3”, based on Guo et al.'s best practices for reporting pre-clinical data, which

$$A U C . 3 = \frac{{AUC}_{log 2} - ( {log}_{2} ( Initial\_Volume ) * Days\_on \_Study )}{Days\_on {\_Study}^{2}}$$

reflects area under a continuous growth curve and accounts for time on study (Guo S, Jiang X, Mao B, et al. The design, analysis, and application of mouse clinical trials in oncology drug development. BMC Cancer 2019; 19:718).

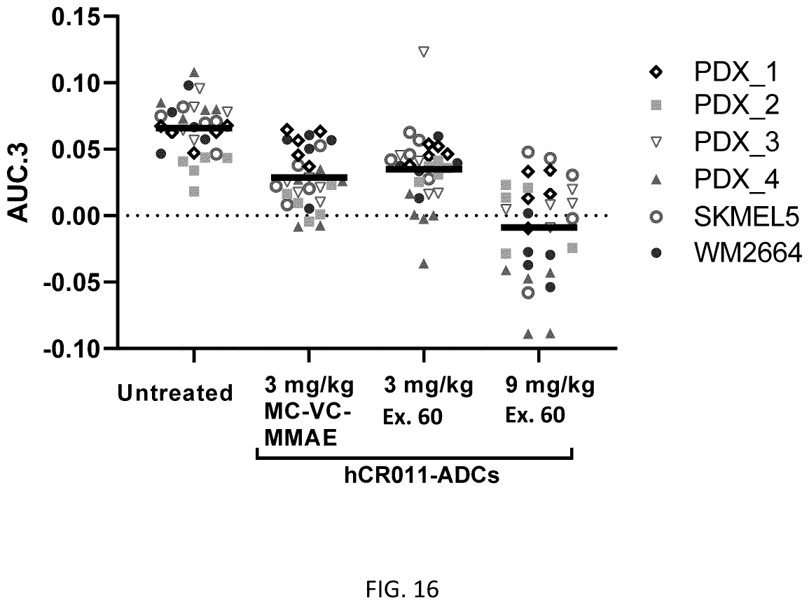

The data (see FIG. 16) show that hCR011 ADCs comprising hydrophilic auristatin Ex. 60 (compound 3.1 wherein L is hCR011) dosed at 3 mg/kg and 9 mg/kg have comparable and enhanced efficacy, respectively, to ADCs comprising MC-VC-MMAE dosed at 3 mg/kg.

### Example 97. Toxicity Determinations of Ligand-Drug Conjugate Compounds

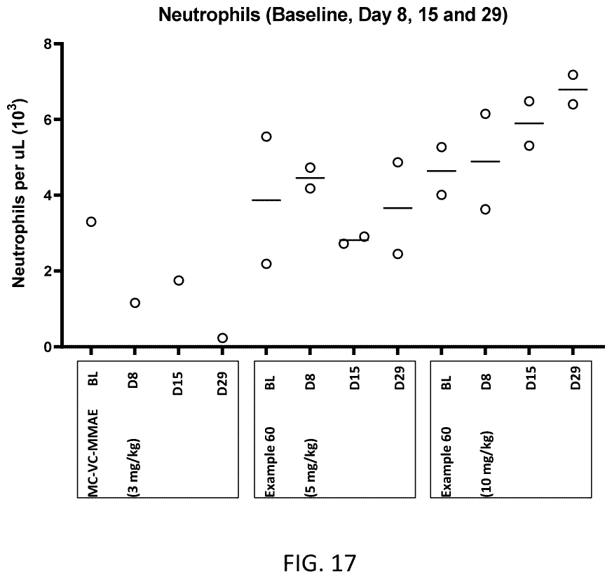

In vivo safety studies were done in naive cynomolgus monkeys. Studies had 1 or 2 animals/group/timepoint of hCR011-MC-VC-MMAE (DAR=4) or hCR011-Example 60 (DAR=4). Animals were administered four weekly doses of 3, 5, or 10 mg/kg of ADC via intravenous (slow bolus) injection. Baseline measurements for hematology and clinical chemistry parameters were taken from blood drawn at one or two time points prior to dosing, and post-dose timepoints were collected at day 8, and also on days 15 and 29 (hematology only). Results are shown in FIGS. 17 to 20.

FIG. 17 demonstrates that neutrophil counts post-administration of hydrophilic auristatin conjugate hCR011-Example 60 at 5 and 10 mg/kg were maintained at baseline levels, whereas conjugate hCR011-MC-VC-MMAE at 3 mg/kg shows a decrease relative to baseline on days 8, 15, and 29.

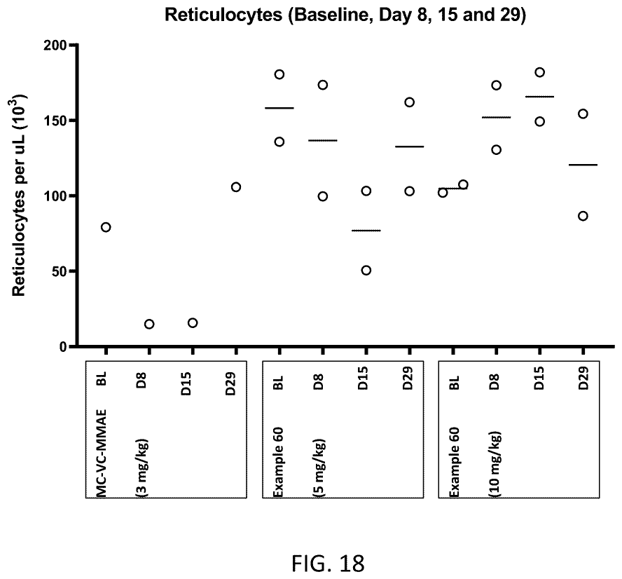

FIG. 18 demonstrates that loss of reticulocytes is greatly improved after administering hydrophilic auristatin conjugate hCR011-Example 60 at 5 and 10 mg/kg compared to conjugate hCR011-MC-VC-MMAE at 3 mg/kg.

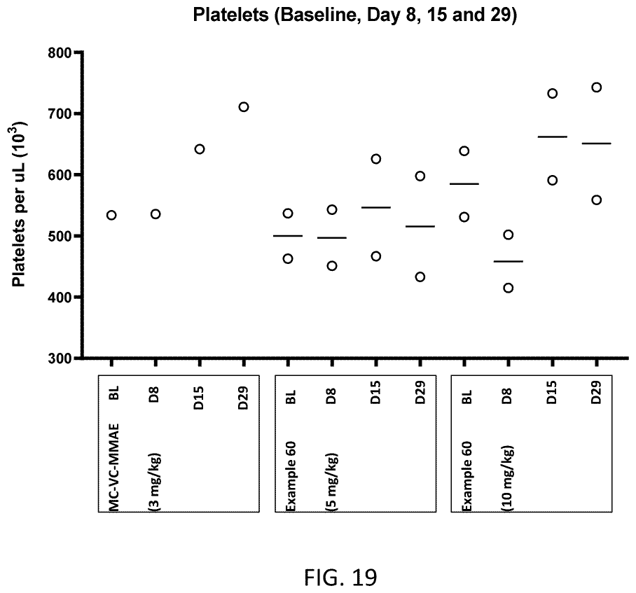

FIG. 19 shows that administering hydrophilic auristatin conjugate hCR011-Example 60 does not significantly impact platelet counts at higher dose-levels of 5 and 10 mg/kg compared to conjugate hCR011-MC-VC-MMAE at 3 mg/kg.

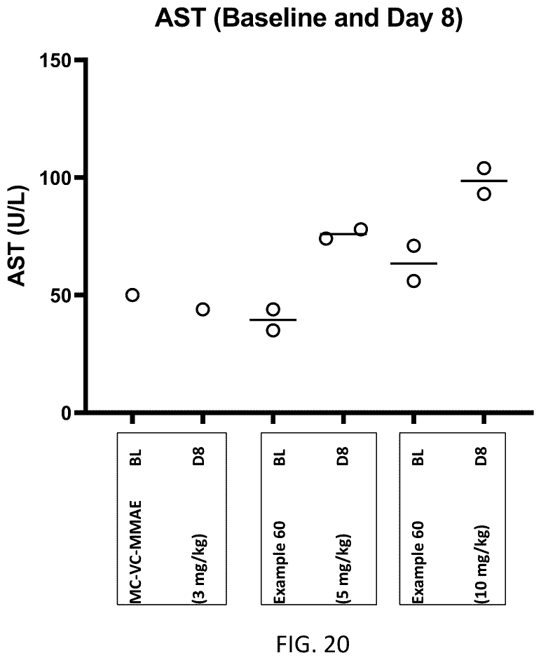

FIG. 20 shows that administering hydrophilic auristatin conjugate hCR011-Example 60 does not significantly impact AST levels at higher dose-levels of 5 and 10 mg/kg compared to conjugate hCR011-MC-VC-MMAE at 3 mg/kg.

### Example 98. Toxicity Determinations of Ligand-Drug Conjugate Compounds

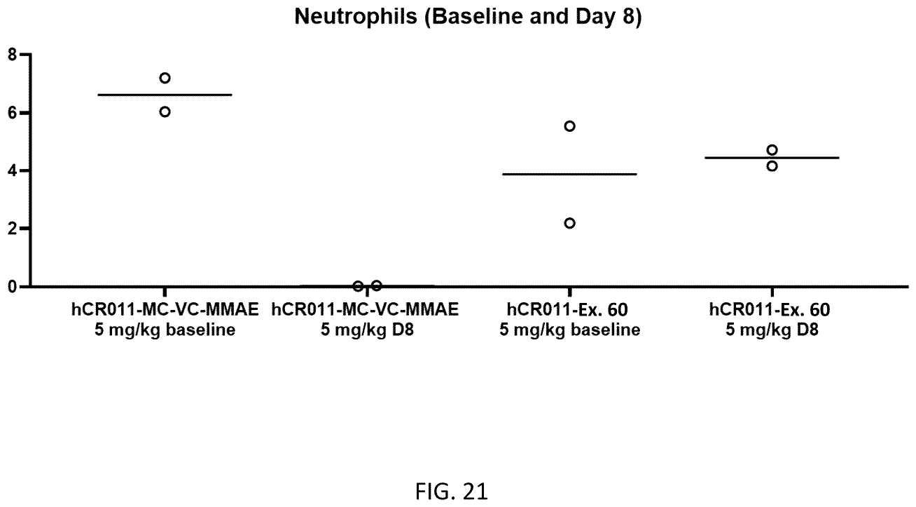

In vivo toxicology studies were done in naive cynomolgus monkeys. Studies had 2 animals/group/timepoint of hCR011-MC-VC-MMAE (DAR=4) or hCR011-Example 60 (DAR=4). Animals were administered four weekly doses of 5 mg/kg of ADC via intravenous (slow bolus) injection. Baseline measurements for hematology parameters were collected and results are shown in FIG. 21. The 5 mg/kg dose of hCR011-MC-VC-MMAE was not tolerated and dosing could not be completed for both animals whereas animals treated with 5 mg/kg of hCR011-Example 60 completed the study as planned.

FIG. 21 shows neutrophil counts from blood samples taken prior to dosing and post-dose on Day 8. Neutrophil counts post-administration of hydrophilic auristatin conjugate hCR011-Example 60 dosed at 5 mg/kg were maintained at baseline levels, whereas conjugate hCR011-MC-VC-MMAE at 5 mg/kg showed a decrease relative to baseline.

While the foregoing written description of the compounds, uses, and methods described herein enables one of ordinary skill to make and use the compounds, uses, and methods described herein, those of ordinary skill will understand and appreciate the existence of variations, combinations, and equivalents of the specific embodiment, method, and examples herein. The compounds, uses, and methods provided herein should therefore not be limited by the above-described embodiments, methods, or examples, but rather encompasses all embodiments and methods within the scope and spirit of the compounds, uses, and methods provided herein.

All references disclosed herein are incorporated by reference in their entirety.

Unless otherwise indicated, all sequences and SEQ IDs referenced in the application correspond to the appropriately numbered sequence in the table below.

The Table of Sequences below provides exemplary antibody sequences consistent with the present disclosure. In this table, all targets correspond to human orthologs unless otherwise specified.

| SEQ | | |
|---|---|---|
| ID | | |
| NO | Description | Sequence |
| 1 | Anti-PD-L1 | QVQLVQSGAEVKKPGSSVKVSCKTSGDTFSTAAISWVRQAPGQGLEWMGGIIPIFGKAHY |
| | HC | AQKFQGRVTITADESTSTAYMELSSLRSEDTAVYFCARKFHFVSGSPFGMDVWGQGTTVT |
| | | VSSASTKGPSVFPLAPSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGK |
| 2 | Anti-PD-L1 | QVQLVQSGAEVKKPGSSVKVSCKTSGDTFSTAAISWVRQAPGQGLEWMGGIIPIFGKAHY |
| | HC v2 | AQKFQGRVTITADESTSTAYMELSSLRSEDTAVYFCARKFHFVSGSPFGMDVWGQGTTVT |
| | | VSSASTKGPSVFPLAPSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPG |
| 3 | Anti-PD-L1 | QVQLVQSGAEVKKPGSSVKVSCKTSGDTFSTAAISWVRQAPGQGLEWMGGIIPIFGKAHY |
| | LALA HC | AQKFQGRVTITADESTSTAYMELSSLRSEDTAVYFCARKFHFVSGSPFGMDVWGQGTTVT |
| | | VSSASTKGPSVFPLAPSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSCDKTHTCPPCPAPEA |
| | | AGGPSVFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGK |
| 4 | Anti-PD-L1 | QVQLVQSGAEVKKPGSSVKVSCKTSGDTFSTAAISWVRQAPGQGLEWMGGIIPIFGKAHY |
| | LALA HC v2 | AQKFQGRVTITADESTSTAYMELSSLRSEDTAVYFCARKFHFVSGSPFGMDVWGQGTTVT |
| | | VSSASTKGPSVFPLAPSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSCDKTHTCPPCPAPEA |
| | | AGGPSVFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPG |
| 5 | Anti-PD-L1 | EIVLTQSPATLSLSPGERATLSCRASQSVSSYLAWYQQKPGQAPRLLIYDASNRATGIPA |
| | LC | RFSGSGSGTDFTLTISSLEPEDFAVYYCQQRSNWPTFGQGTKVEIKRTVAAPSVFIFPPS |
| | | DEQLKSGTASVVCLLNNFYPREAKVQWKVDNALQSGNSQESVTEQDSKDSTYSLSSTLTL |
| | | SKADYEKHKVYACEVTHQGLSSPVTKSENRGEC |
| 6 | Anti-PD-L1 | QVQLVQSGAEVKKPGSSVKVSCKTSGDTFSTAAISWVRQAPGQGLEWMGGIIPIFGKAHY |
| | (engineered | AQKFQGRVTITADESTSTAYMELSSLRSEDTAVYFCARKFHFVSGSPFGMDVWGQGTTVT |
| | cysteines) | VSSASTKGPSVFPLAPSSKSTSGGTAALGCLVKDYFPEPVTVSWNSGALTSGVHTFPAVL |
| | LALA HC | QSSGLYSLSSVVTVPSSSLGTQTYICNVNHKPSNTKVDKKVEPKSCDKTHTCPPCPAPEA |
| | | AGGPCVFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGK |
| 7 | Anti-PD-L1 | QVQLVQSGAEVKKPGSSVKVSCKTSGDTESTAAISWVRQAPGQGLEWMGGIIPIFGKAHY |
| | (engineered | AQKFQGRVTITADESTSTAYMELSSLRSEDTAVYFCARKFHFVSGSPFGMDVWGQGTTVT |
| | cysteines) | VSSASTKGPSVFPLAPSSKSTSGGTAALGCLVKDYFPEPVTVSWNSGALTSGVHTFPAVL |
| | LALA HC v2 | QSSGLYSLSSVVTVPSSSLGTQTYICNVNHKPSNTKVDKKVEPKSCDKTHTCPPCPAPEA |
| | | AGGPCVFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPG |
| 8 | Anti-PD-L1 | QVQLVQSGAEVKKPGSSVKVSCKTSGDTESTAAISWVRQAPGQGLEWMGGIIPIFGKAHY |
| | mIgG2a LALA | AQKFQGRVTITADESTSTAYMELSSLRSEDTAVYFCARKFHFVSGSPFGMDVWGQGTTVT |
| | HC | VSSAKTTAPSVYPLAPVCGDTTGSSVTLGCLVKGYFPEPVTLTWNSGSLSSGVHTFPAVL |
| | | QSDLYTLSSSVTVTSSTWPSQSITCNVAHPASSTKVDKKIEPRGPTIKPCPPCKCPAPNA |
| | | AGGPSVFIFPPKIKDVLMISLSPIVTCVVVDVSEDDPDVQISWFVNNVEVHTAQTQTHRE |
| | | DYNSTLRVVSALPIQHQDWMSGKEFKCKVNNKDLPAPIERTISKPKGSVRAPQVYVLPPP |
| | | EEEMTKKQVTLTCMVTDFMPEDIYVEWTNNGKTELNYKNTEPVLDSDGSYFMYSKLRVEK |
| | | KNWVERNSYSCSVVHEGLHNHHTTKSFSRTPGK |
| 9 | Anti-PD-L1 | QVQLVQSGAEVKKPGSSVKVSCKTSGDTFSTAAISWVRQAPGQGLEWMGGIIPIFGKAHY |
| | mIgG2a LALA | AQKFQGRVTITADESTSTAYMELSSLRSEDTAVYFCARKFHFVSGSPFGMDVWGQGTTVT |
| | HC v2 | VSSAKTTAPSVYPLAPVCGDTTGSSVTLGCLVKGYFPEPVTLTWNSGSLSSGVHTFPAVL |
| | | QSDLYTLSSSVTVTSSTWPSQSITCNVAHPASSTKVDKKIEPRGPTIKPCPPCKCPAPNA |
| | | AGGPSVFIFPPKIKDVLMISLSPIVTCVVVDVSEDDPDVQISWFVNNVEVHTAQTQTHRE |
| | | DYNSTLRVVSALPIQHQDWMSGKEFKCKVNNKDLPAPIERTISKPKGSVRAPQVYVLPPP |
| | | EEEMTKKQVTLTCMVTDFMPEDIYVEWTNNGKTELNYKNTEPVLDSDGSYFMYSKLRVEK |
| | | KNWVERNSYSCSVVHEGLHNHHTTKSFSRTPG |
| 10 | Anti-PD-L1 | EIVLTQSPATLSLSPGERATLSCRASQSVSSYLAWYQQKPGQAPRLLIYDASNRATGIPA |
| | mK LC | RFSGSGSGTDFTLTISSLEPEDFAVYYCQQRSNWPTFGQGTKVEIKRADAAPTVSIFPPS |
| | | SEQLTSGGASVVCFLNNFYPKDINVKWKIDGSERQNGVLNSWTDQDSKDSTYSMSSTLTL |
| | | TKDEYERHNSYTCEATHKTSTSPIVKSFNRNEC |
| 11 | Anti-PD-L1 | QVQLVQSGAEVKKPGSSVKVSCKTSGDTFSTAAISWVRQAPGQGLEWMGGIIPIFGKAHY |
| | mIgG2a | AQKFQGRVTITADESTSTAYMELSSLRSEDTAVYFCARKFHFVSGSPFGMDVWGQGTTVT |
| | (engineered | VSSAKTTAPSVYPLAPVCGDTTGSSVTLGCLVKGYFPEPVTLTWNSGSLSSGVHTFPAVL |
| | cysteines) | QSDLYTLSSSVTVTSSTWPSQSITCNVAHPASSTKVDKKIEPRGPTIKPCPPCKCPAPNA |
| | LALA HC | AGGPCVFIFPPKIKDVLMISLSPIVTCVVVDVSEDDPDVQISWFVNNVEVHTAQTQTHRE |
| | | DYNSTLRVVSALPIQHQDWMSGKEFKCKVNNKDLPAPIERTISKPKGSVRAPQVYVLPPP |
| | | EEEMTKKQVTLTCMVTDFMPEDIYVEWTNNGKTELNYKNTEPVLDSDGSYFMYSKLRVEK |
| | | KNWVERNSYSCSVVHEGLHNHHTTKSFSRTPGK |
| 12 | Anti-PD-L1 | QVQLVQSGAEVKKPGSSVKVSCKTSGDTFSTAAISWVRQAPGQGLEWMGGIIPIFGKAHY |
| | mIgG2a | AQKFQGRVTITADESTSTAYMELSSLRSEDTAVYFCARKFHFVSGSPFGMDVWGQGTTVT |
| | (engineered | VSSAKTTAPSVYPLAPVCGDTTGSSVTLGCLVKGYFPEPVTLTWNSGSLSSGVHTFPAVL |
| | cysteines) | QSDLYTLSSSVTVISSTWPSQSITCNVAHPASSTKVDKKIEPRGPTIKPCPPCKCPAPNA |
| | LALA HC v2 | AGGPCVFIFPPKIKDVLMISLSPIVTCVVVDVSEDDPDVQISWFVNNVEVHTAQTQTHRE |
| | | DYNSTLRVVSALPIQHQDWMSGKEFKCKVNNKDLPAPIERTISKPKGSVRAPQVYVLPPP |
| | | EEEMTKKQVTLTCMVTDFMPEDIYVEWTNNGKTELNYKNTEPVLDSDGSYFMYSKLRVEK |
| | | KNWVERNSYSCSVVHEGLHNHHTTKSFSRTPG |
| 13 | Anti-PD-L1 | TAAIS |
| | CDR-H1 | |
| 14 | Anti-PD-L1 | GIIPIFGKAHYAQKFQG |
| | CDR-H2 | |
| 15 | Anti-PD-L1 | KFHFVSGSPFGMDV |
| | CDR-H3 | |
| 16 | Anti-PD-L1 | RASQSVSSYLA |
| | CDR-L1 | |
| 17 | Anti-PD-L1 | DASNRAT |
| | CDR-L2 | |
| 18 | Anti-PD-L1 | QQRSNWPT |
| | CDR-L3 | |
| 19 | Anti-PD-L1 | QVQLVQSGAEVKKPGSSVKVSCKTSGDTFSTAAISWVRQAPGQGLEWMGGIIPIFGKAHY |
| | VH | AQKFQGRVTITADESTSTAYMELSSLRSEDTAVYFCARKFHFVSGSPFGMDVWGQGTTVT |
| | | VSS |
| 20 | Anti-PD-L1 | EIVLTQSPATLSLSPGERATLSCRASQSVSSYLAWYQQKPGQAPRLLIYDASNRATGIPA |
| | VL | RFSGSGSGTDFTLTISSLEPEDFAVYYCQQRSNWPTFGQGTKVEIK |
| 21 | Anti-EphA2 | HYMMA |
| | CDR-H1 | |
| 22 | Anti-EphA2 | RIGPSGGPTHYADSVKG |
| | CDR-H2 | |
| 23 | Anti-EphA2 | YDSGYDYVAVAGPAEYFQH |
| | CDR-H3 | |
| 24 | Anti-EphA2 | RASQSISTWLA |
| | CDR-L1 | |
| 25 | Anti-EphA2 | KASNLHT |
| | CDR-L2 | |
| 26 | Anti-EphA2 | QQYNSYSRT |
| | CDR-L3 | |
| 27 | Anti-EphA2 | EVQLLESGGGLVQPGGSLRLSCAASGFTFSHYMMAWVRQAPGKGLEWVSRIGPSGGPTHY |
| | VH | ADSVKGRFTISRDNSKNTLYLQMNSLRAEDTAVYYCAGYDSGYDYVAVAGPAEYFQHWGQ |
| | | GTLVTVSS |
| 28 | Anti-EphA2 | DIQMTQSPSSLSASVGDRVTITCRASQSISTWLAWYQQKPGKAPKLLIYKASNLHTGVPS |
| | VL | RFSGSGSGTEFSLTISGLQPDDFATYYCQQYNSYSRTFGQGTKVEIK |
| 29 | Anti-EphA2 | EVQLLESGGGLVQPGGSLRLSCAASGFTFSHYMMAWVRQAPGKGLEWVSRIGPSGGPTHY |
| | HC | ADSVKGRFTISRDNSKNTLYLQMNSLRAEDTAVYYCAGYDSGYDYVAVAGPAEYFQHWGQ |
| | | GTLVTVSSASTKGPSVFPLAPSSKSTSGGTAALGCLVKDYFPEPVTVSWNSGALTSGVHT |
| | | FPAVLQSSGLYSLSSVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQVY |
| | | TLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGK |
| 30 | Anti-EphA2 | EVQLLESGGGLVQPGGSLRLSCAASGFTFSHYMMAWVRQAPGKGLEWVSRIGPSGGPTHY |
| | HC v2 | ADSVKGRFTISRDNSKNTLYLQMNSLRAEDTAVYYCAGYDSGYDYVAVAGPAEYFQHWGQ |
| | | GTLVTVSSASTKGPSVFPLAPSSKSTSGGTAALGCLVKDYFPEPVTVSWNSGALTSGVHT |
| | | FPAVLQSSGLYSLSSVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQVY |
| | | TLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPG |
| 31 | Anti-EphA2 | DIQMTQSPSSLSASVGDRVTITCRASQSISTWLAWYQQKPGKAPKLLIYKASNLHTGVPS |
| | LC | RFSGSGSGTEFSLTISGLQPDDFATYYCQQYNSYSRTFGQGTKVEIKRTVAAPSVFIFPP |
| | | SDEQLKSGTASVVCLLNNFYPREAKVQWKVDNALQSGNSQESVTEQDS |
| | | KDSTYSLSSTLTLSKADYEKHKVYACEVTHQGLSSPVTKSFNRGEC |
| 32 | Anti-EphA2 | EVQLLESGGGLVQPGGSLRLSCAASGFTFSHYMMAWVRQAPGKGLEWVSRIGPSGGPTHY |
| | mIgG2a HC | ADSVKGRFTISRDNSKNTLYLQMNSLRAEDTAVYYCAGYDSGYDYVAVAGPAEYFQHWGQ |
| | | GTLVTVSSAKTTAPSVYPLAPVCGDTTGSSVTLGCLVKGYFPEPVTLTWNSGSLSSGVHT |
| | | FPAVLQSDLYTLSSSVTVTSSTWPSQSITCNVAHPASSTKVDKKIEPRGPTIKPCPPCKC |
| | | PAPNLLGGPSVFIFPPKIKDVLMISLSPIVTCVVVDVSEDDPDVQISWFVNNVEVHTAQT |
| | | QTHREDYNSTLRVVSALPIQHQDWMSGKEFKCKVNNKDLPAPIERTISKPKGSVRAPQVY |
| | | VLPPPEEEMTKKQVTLTCMVTDFMPEDIYVEWTNNGKTELNYKNTEPVLDSDGSYFMYSK |
| | | LRVEKKNWVERNSYSCSVVHEGLHNHHTTKSFSRTPGK |
| 33 | Anti-EphA2 | EVQLLESGGGLVQPGGSLRLSCAASGFTFSHYMMAWVRQAPGKGLEWVSRIGPSGGPTHY |
| | mIgG2a HC | ADSVKGRFTISRDNSKNTLYLQMNSLRAEDTAVYYCAGYDSGYDYVAVAGPAEYFQHWGQ |
| | v2 | GTLVTVSSAKTTAPSVYPLAPVCGDTTGSSVTLGCLVKGYFPEPVTLTWNSGSLSSGVHT |
| | | FPAVLQSDLYTLSSSVTVTSSTWPSQSITCNVAHPASSTKVDKKIEPRGPTIKPCPPCKC |
| | | PAPNLLGGPSVFIFPPKIKDVLMISLSPIVTCVVVDVSEDDPDVQISWFVNNVEVHTAQT |
| | | QTHREDYNSTLRVVSALPIQHQDWMSGKEFKCKVNNKDLPAPIERTISKPKGSVRAPQVY |
| | | VLPPPEEEMTKKQVTLTCMVTDFMPEDIYVEWTNNGKTELNYKNTEPVLDSDGSYFMYSK |
| | | LRVEKKNWVERNSYSCSVVHEGLHNHHTTKSFSRTPG |
| 34 | Anti-EphA2 | DIQMTQSPSSLSASVGDRVTITCRASQSISTWLAWYQQKPGKAPKLLIYKASNLHTGVPS |
| | mIgG2a LC | RFSGSGSGTEFSLTISGLQPDDFATYYCQQYNSYSRTFGQGTKVEIKRADAAPTVSIFPP |
| | | SSEQLTSGGASVVCFLNNFYPKDINVKWKIDGSERQNGVLNSWTDQDSKDSTYSMSSTLT |
| | | LTKDEYERHNSYTCEATHKTSTSPIVKSFNRNEC |
| 35 | Anti-EphA2 | EVQLLESGGGLVQPGGSLRLSCAASGFTFSHYMMAWVRQAPGKGLEWVSRIGPSGGPTHY |
| | mIgG2a | ADSVKGRFTISRDNSKNTLYLQMNSLRAEDTAVYYCAGYDSGYDYVAVAGPAEYFQHWGQ |
| | LALAPG HC | GTLVTVSSAKTTAPSVYPLAPVCGDTTGSSVTLGCLVKGYFPEPVTLTWNSGSLSSGVHT |
| | | FPAVLQSDLYTLSSSVTVTSSTWPSQSITCNVAHPASSTKVDKKIEPRGPTIKPCPPCKC |
| | | PAPNAAGGPSVFIFPPKIKDVLMISLSPIVTCVVVDVSEDDPDVQISWFVNNVEVHTAQT |
| | | QTHREDYNSTLRVVSALPIQHQDWMSGKEFKCKVNNKDLGAPIERTISKPKGSVRAPQVY |
| | | VLPPPEEEMTKKQVTLTCMVTDFMPEDIYVEWTNNGKTELNYKNTEPVLDSDGSYFMYSK |
| | | LRVEKKNWVERNSYSCSVVHEGLHNHHTTKSFSRTPGK |
| 36 | Anti-EphA2 | EVQLLESGGGLVQPGGSLRLSCAASGFTFSHYMMAWVRQAPGKGLEWVSRIGPSGGPTHY |
| | mIgG2a | ADSVKGRFTISRDNSKNTLYLQMNSLRAEDTAVYYCAGYDSGYDYVAVAGPAEYFQHWGQ |
| | LALAPG HC | GTLVTVSSAKTTAPSVYPLAPVCGDTTGSSVTLGCLVKGYFPEPVTLTWNSGSLSSGVHT |
| | v2 | FPAVLQSDLYTLSSSVTVTSSTWPSQSITCNVAHPASSTKVDKKIEPRGPTIKPCPPCKC |
| | | PAPNAAGGPSVFIFPPKIKDVLMISLSPIVTCVVVDVSEDDPDVQISWFVNNVEVHTAQT |
| | | QTHREDYNSTLRVVSALPIQHQDWMSGKEFKCKVNNKDLGAPIERTISKPKGSVRAPQVY |
| | | VLPPPEEEMTKKQVTLTCMVTDFMPEDIYVEWTNNGKTELNYKNTEPVLDSDGSYFMYSK |
| | | LRVEKKNWVERNSYSCSVVHEGLHNHHTTKSFSRTPG |
| 37 | Anti-EphA2 | DIQMTQSPSSLSASVGDRVTITCRASQSISTWLAWYQQKPGKAPKLLIYKASNLHTGVPS |
| | mIgG2a | RFSGSGSGTEFSLTISGLQPDDFATYYCQQYNSYSRTFGQGTKVEIKRADAAPTVSIFPP |
| | LALAPG LC | SSEQLTSGGASVVCFLNNFYPKDINVKWKIDGSERQNGVLNSWTDQDSKDSTYSMSSTLT |
| | | LTKDEYERHNSYTCEATHKTSTSPIVKSFNRNEC |
| 38 | Anti-CD30 | DYYIT |
| | CDR-H1 | |
| 39 | Anti-CD30 | WIYPGSGNTKYNEKFKG |
| | CDR-H2 | |
| 40 | Anti-CD30 | YGNYWFAY |
| | CDR-H3 | |
| 41 | Anti-CD30 | KASQSVDFDGDSYMN |
| | CDR-L1 | |
| 42 | Anti-CD30 | AASNLES |
| | CDR-L2 | |
| 43 | Anti-CD30 | QQSNEDPWT |
| | CDR-L3 | |
| 44 | Anti-CD30 | QIQLQQSGPEVVKPGASVKISCKASGYTFTDYYITWVKQKPGQGLEWIGWIYPGSGNTKY |
| | VH | NEKFKGKATLTVDTSSSTAFMQLSSLTSEDTAVYFCANYGNYWFAYWGQGTQVTVSA |
| 45 | Anti-CD30 | DIVLTQSPASLAVSLGQRATISCKASQSVDFDGDSYMNWYQQKPGQPPKVLIYAASNLES |
| | VL | GIPARFSGSGSGTDFTLNIHPVEEEDAATYYCQQSNEDPWTFGGGTKLEIK |
| 46 | Anti-CD30 | QIQLQQSGPEVVKPGASVKISCKASGYTFTDYYITWVKQKPGQGLEWIGWIYPGSGNTKY |
| | HC | NEKFKGKATLTVDTSSSTAFMQLSSLTSEDTAVYFCANYGNYWFAYWGQGTQVTVSAAST |
| | | KGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPCPAPELLGGPSV |
| | | FLFPPKPKDTLMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYTLPPSRDELTK |
| | | NQVSLTCLVKGFYPSDIAVEWESNGQPENNYKTTPPVLDSDGSFFLYSKLTVDKSRWQQG |
| | | NVFSCSVMHEALHNHYTQKSLSLSPGK |
| 47 | Anti-CD30 | QIQLQQSGPEVVKPGASVKISCKASGYTFTDYYITWVKQKPGQGLEWIGWIYPGSGNTKY |
| | HC v2 | NEKFKGKATLTVDTSSSTAFMQLSSLTSEDTAVYFCANYGNYWFAYWGQGTQVTVSAAST |
| | | KGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPCPAPELLGGPSV |
| | | FLFPPKPKDTLMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYTLPPSRDELTK |
| | | NQVSLTCLVKGFYPSDIAVEWESNGQPENNYKTTPPVLDSDGSFFLYSKLTVDKSRWQQG |
| | | NVFSCSVMHEALHNHYTQKSLSLSPG |
| 48 | Anti-CD30 | DIVLTQSPASLAVSLGQRATISCKASQSVDFDGDSYMNWYQQKPGQPPKVLIYAASNLES |
| | LC | GIPARFSGSGSGTDFTLNIHPVEEEDAATYYCQQSNEDPWTFGGGTKLEIKR |
| | | TVAAPSVFIFPPSDEQLKSGTASVVCLLNNFYPREAKVQWKVDNALQSGNSQESVTEQDS |
| | | KDSTYSLSSTLTLSKADYEKHKVYACEVTHQGLSSPVTKSENRGEC |
| 49 | Anti-CD228 | SGYWN |
| | CDR-H1 | |
| 50 | Anti-CD228 | YISDSGITYYNPSLKS |
| | CDR-H2 | |
| 51 | Anti-CD228 | RTLATYYAMDY |
| | CDR-H3 | |
| 52 | Anti-CD228 | RASQSLVHSDGNTYLH |
| | CDR-L1 | |
| 53 | Anti-CD228 | RVSNRFS |
| | CDR-L2 | |
| 54 | Anti-CD228 | SQSTHVPPT |
| | CDR-L3 | |
| 55 | Anti-CD228 | QVQLQESGPGLVKPSETLSLTCTVSGDSITSGYWNWIRQPPGKGLEYIGYISDSGITYYN |
| | VH | PSLKSRVTISRDTSKNQYSLKLSSVTAADTAVYYCARRTLATYYAMDYWGQGTLVTVSS |
| 56 | Anti-CD228 | DFVMTQSPLSLPVTLGQPASISCRASQSLVHSDGNTYLHWYQQRPGQSPRLLIYRVSNRF |
| | VL | SGVPDRFSGSGSGTDFTLKISRVEAEDVGVYYCSQSTHVPPTFGQGTKLEIK |
| 57 | Anti-CD228 | QVQLQESGPGLVKPSETLSLTCTVSGDSITSGYWNWIRQPPGKGLEYIGYISDSGITYYN |
| | HC | PSLKSRVTISRDTSKNQYSLKLSSVTAADTAVYYCARRTLATYYAMDYWGQGTLVTVSSA |
| | | STKGPSVFPLAPSSKSTSGGTAALGCLVKDYFPEPVTVSWNSGALTSGVHTFPAVLQSSG |
| | | LYSLSSVVTVPSSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHNAKTKPREEQYNS |
| | | TYRVVSVLTVLHQDWLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSFFLYSKLTVDKSRWQ |
| | | QGNVFSCSVMHEALHNHYTQKSLSLSPGK |
| 58 | Anti-CD228 | QVQLQESGPGLVKPSETLSLTCTVSGDSITSGYWNWIRQPPGKGLEYIGYISDSGITYYN |
| | HC v2 | PSLKSRVTISRDTSKNQYSLKLSSVTAADTAVYYCARRTLATYYAMDYWGQGTLVTVSSA |
| | | STKGPSVFPLAPSSKSTSGGTAALGCLVKDYFPEPVTVSWNSGALTSGVHTFPAVLQSSG |
| | | LYSLSSVVTVPSSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHNAKTKPREEQYNS |
| | | TYRVVSVLTVLHQDWLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSFFLYSKLTVDKSRWQ |
| | | QGNVFSCSVMHEALHNHYTQKSLSLSPG |
| 59 | Anti-CD228 | DFVMTQSPLSLPVTLGQPASISCRASQSLVHSDGNTYLHWYQQRPGQSPRLLIYRVSNRF |
| | LC | SGVPDRFSGSGSGTDFTLKISRVEAEDVGVYYCSQSTHVPPTFGQGTKLEIKRTVAAPSV |
| | | FIFPPSDEQLKSGTASVVCLLNNFYPREAKVQWKVDNALQSGNSQESVTEQDSKDSTYSL |
| | | SSTLTLSKADYEKHKVYACEVTHQGLSSPVTKSENRGEC |
| 60 | Anti-CD228 | QVQLQESGPGLVKPSETLSLTCTVSGDSITSGYWNWIRQPPGKGLEYIGYISDSGITYYN |
| | LALAKA HC | PSLKSRVTISRDTSKNQYSLKLSSVTAADTAVYYCARRTLATYYAMDYWGQGTLVTVSSA |
| | | STKGPSVFPLAPSSKSTSGGTAALGCLVKDYFPEPVTVSWNSGALTSGVHTFPAVLQSSG |
| | | LYSLSSVVTVPSSSLGTQTYICNVNHKPSNTKVDKKVEPKSCDKTHTCPPCPAPEAAGGP |
| | | SVFLFPPKPKDTLMISRTPEVTCVVVDVSHEDPEVKFNWYVDGVEVHNAKTKPREEQYNS |
| | | TYRVVSVLTVLHQDWLNGKEYKCAVSNKALPAPIEKTISKAKGQPREPQVYTLPPSRDEL |
| | | TKNQVSLTCLVKGFYPSDIAVEWESNGQPENNYKTTPPVLDSDGSFFLYSKLTVDKSRWQ |
| | | QGNVFSCSVMHEALHNHYTQKSLSLSPGK |
| 61 | Anti-CD228 | QVQLQESGPGLVKPSETLSLTCTVSGDSITSGYWNWIRQPPGKGLEYIGYISDSGITYYN |
| | LALAKA HC | PSLKSRVTISRDTSKNQYSLKLSSVTAADTAVYYCARRTLATYYAMDYWGQGTLVTVSSA |
| | v2 | STKGPSVFPLAPSSKSTSGGTAALGCLVKDYFPEPVTVSWNSGALTSGVHTFPAVLQSSG |
| | | LYSLSSVVTVPSSSLGTQTYICNVNHKPSNTKVDKKVEPKSCDKTHTCPPCPAPEAAGGP |
| | | SVFLFPPKPKDTLMISRTPEVTCVVVDVSHEDPEVKENWYVDGVEVHNAKTKPREEQYNS |
| | | TYRVVSVLTVLHQDWLNGKEYKCAVSNKALPAPIEKTISKAKGQPREPQVYTLPPSRDEL |
| | | TKNQVSLTCLVKGFYPSDIAVEWESNGQPENNYKTTPPVLDSDGSFFLYSKLTVDKSRWQ |
| | | QGNVFSCSVMHEALHNHYTQKSLSLSPG |
| 62 | Anti-CD228 | DFVMTQSPLSLPVTLGQPASISCRASQSLVHSDGNTYLHWYQQRPGQSPRLLIYRVSNRF |
| | LALAKA LC | SGVPDRFSGSGSGTDFTLKISRVEAEDVGVYYCSQSTHVPPTFGQGTKLEIKRTVAAPSV |
| | | FIFPPSDEQLKSGTASVVCLLNNFYPREAKVQWKVDNALQSGNSQESVTEQDSKDSTYSL |
| | | SSTLTLSKADYEKHKVYACEVTHQGLSSPVTKSENRGEC |
| 63 | Anti-avB6 | DYNVN |
| | HC CDR-H1 | |
| 64 | Anti-AvB6 | VINPKYGTTRYNQKFKG |
| | HC CDR-H2 | |
| 65 | Anti-AvB6 | GLNAWDY |
| | HC CDR-H3 | |
| 66 | Anti-AvB6 | GASENIYGALN |
| | LG CDR-L1 | |
| 67 | Anti-AvB6 | GATNLED |
| | LG CDR-L2 | |
| 68 | Anti-AvB6 | QNVLTTPYT |
| | LG CDR-L3 | |
| 69 | Anti-AvB6 | QFQLVQSGAEVKKPGASVKVSCKASGYSFTDYNVNWVRQAPGQGLEWIGVINPKYGTTRY |
| | HC VH | NQKFKGRATLTVDKSTSTAYMELSSLRSEDTAVYYCTRGLNAWDYWGQGTLVTVSS |
| 70 | Anti-AvB6 | DIQMTQSPSSLSASVGDRVTITCGASENIYGALNWYQQKPGKAPKLLIYGATNLEDGVPS |
| | LG VL | RFSGSGSGRDYTFTISSLQPEDIATYYCQNVLTTPYTFGQGTKLEIK |
| 71 | Anti-AvB6 | QFQLVQSGAEVKKPGASVKVSCKASGYSFTDYNVNWVRQAPGQGLEWIGVINPKYGTTRY |
| | HC | NQKFKGRATLTVDKSTSTAYMELSSLRSEDTAVYYCTRGLNAWDYWGQGTLVTVSSASTK |
| | | GPSVFPLAPSSKSTSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPPCPAPELLGGPSVF |
| | | LFPPKPKDTLMISRTPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQVYTLPPSRDELTKN |
| | | QVSLTCLVKGFYPSDIAVEWESNGQPENNYKTTPPVLDSDGSFFLYSKLTVDKSRWQQGN |
| | | VFSCSVMHEALHNHYTQKSLSLSPGK |
| 72 | Anti-AvB6 | QFQLVQSGAEVKKPGASVKVSCKASGYSFTDYNVNWVRQAPGQGLEWIGVINPKYGTTRY |
| | HC v2 | NQKFKGRATLTVDKSTSTAYMELSSLRSEDTAVYYCTRGLNAWDYWGQGTLVTVSSASTK |
| | | GPSVFPLAPSSKSTSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPPCPAPELLGGPSVF |
| | | LFPPKPKDTLMISRTPEVTCVVVDVSHEDPEVKENWYVDGVEVHNAKTKPREEQYNSTYR |
| | | VVSVLTVLHQDWLNGKEYKCKVSNKALPAPIEKTISKAKGQPREPQVYTLPPSRDELTKN |
| | | QVSLTCLVKGFYPSDIAVEWESNGQPENNYKTTPPVLDSDGSFFLYSKLTVDKSRWQQGN |
| | | VFSCSVMHEALHNHYTQKSLSLSPG |
| 73 | Anti-AvB6 | DIQMTQSPSSLSASVGDRVTITCGASENIYGALNWYQQKPGKAPKLLIYGATNLEDGVPS |
| | LG LC | RFSGSGSGRDYTFTISSLQPEDIATYYCQNVLTTPYTFGQGTKLEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 74 | Anti-AvB6 | QFQLVQSGAEVKKPGASVKVSCKASGYSFTDYNVNWVRQAPGQGLEWIGVINPKYGTTRY |
| | LALAKA HC | NQKFKGRATLTVDKSTSTAYMELSSLRSEDTAVYYCTRGLNAWDYWGQGTLVTVSSASTK |
| | | GPSVFPLAPSSKSTSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPPCPAPEAAGGPSVF |
| | | LFPPKPKDTLMISRTPEVTCVVVDVSHEDPEVKFNWYVDGVEVHNAKTKPREEQYNSTYR |
| | | VVSVLTVLHQDWLNGKEYKCAVSNKALPAPIEKTISKAKGQPREPQVYTLPPSRDELTKN |
| | | QVSLTCLVKGFYPSDIAVEWESNGQPENNYKTTPPVLDSDGSFFLYSKLTVDKSRWQQGN |
| | | VFSCSVMHEALHNHYTQKSLSLSPGK |
| 75 | Anti-AvB6 | QFQLVQSGAEVKKPGASVKVSCKASGYSFTDYNVNWVRQAPGQGLEWIGVINPKYGTTRY |
| | LALAKA HC | NQKFKGRATLTVDKSTSTAYMELSSLRSEDTAVYYCTRGLNAWDYWGQGTLVTVSSASTK |
| | v2 | GPSVFPLAPSSKSTSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPPCPAPEAAGGPSVE |
| | | LFPPKPKDTLMISRTPEVTCVVVDVSHEDPEVKFNWYVDGVEVHNAKTKPREEQYNSTYR |
| | | VVSVLTVLHQDWLNGKEYKCAVSNKALPAPIEKTISKAKGQPREPQVYTLPPSRDELTKN |
| | | QVSLTCLVKGFYPSDIAVEWESNGQPENNYKTTPPVLDSDGSFFLYSKLTVDKSRWQQGN |
| | | VFSCSVMHEALHNHYTQKSLSLSPG |
| 76 | Anti-AvB6 | DIQMTQSPSSLSASVGDRVTITCGASENIYGALNWYQQKPGKAPKLLIYGATNLEDGVPS |
| | LG LALAKA | RFSGSGSGRDYTFTISSLQPEDIATYYCQNVLTTPYTFGQGTKLEIKRTVAAPSVFIFPP |
| | LC | SDEQLKSGTASVVCLLNNFYPREAKVQWKVDNALQSGNSQESVTEQDSKDSTYSLSSTLT |
| | | LSKADYEKHKVYACEVTHQGLSSPVTKSENRGEC |
| 77 | Anti-B7H4 | SGSYYWG |
| | CDR-H1 | |
| 78 | Anti-B7H4 | NIYYSGSTYYNPSLRS |
| | CDR-H2 | |
| 79 | Anti-B7H4 | EGSYPNQFDP |
| | CDR-H3 | |
| 80 | Anti-B7H4 | RASQSVSSNLA |
| | CDR-L1 | |
| 81 | Anti-B7H4 | GASTRAT |
| | CDR-L2 | |
| 82 | Anti-B7H4 | QQYHSFPFT |
| | CDR-L3 | |
| 83 | Anti-B7H4 | QLQLQESGPGLVKPSETLSLTCTVSGGSIKSGSYYWGWIRQPPGKGLEWIGNIYYSGSTY |
| | VH | YNPSLRSRVTISVDTSKNQFSLKLSSVTAADTAVYYCAREGSYPNQFDPWGQGTLVTVSS |
| 84 | Anti-B7H4 | EIVMTQSPATLSVSPGERATLSCRASQSVSSNLAWYQQKPGQAPRLLIYGASTRATGIPA |
| | VL | RFSGSGSGTEFTLTISSLQSEDFAVYYCQQYHSFPFTFGGGTKVEIK |
| 85 | Anti-B7H4 | QLQLQESGPGLVKPSETLSLTCTVSGGSIKSGSYYWGWIRQPPGKGLEWIGNIYYSGSTY |
| | HC | YNPSLRSRVTISVDTSKNQFSLKLSSVTAADTAVYYCAREGSYPNQFDPWGQGTLVTVSS |
| | | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKENWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 86 | Anti-B7H4 | QLQLQESGPGLVKPSETLSLTCTVSGGSIKSGSYYWGWIRQPPGKGLEWIGNIYYSGSTY |
| | HC v2 | YNPSLRSRVTISVDTSKNQFSLKLSSVTAADTAVYYCAREGSYPNQFDPWGQGTLVTVSS |
| | | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPG |
| 87 | Anti-B7H4 | EIVMTQSPATLSVSPGERATLSCRASQSVSSNLAWYQQKPGQAPRLLIYGASTRATGIPA |
| | LC | RFSGSGSGTEFTLTISSLQSEDFAVYYCQQYHSFPFTFGGGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSENRGEC |
| 88 | Anti-B7H4 | QLQLQESGPGLVKPSETLSLTCTVSGGSIKSGSYYWGWIRQPPGKGLEWIGNIYYSGSTY |
| | LALAKA HC | YNPSLRSRVTISVDTSKNQFSLKLSSVTAADTAVYYCAREGSYPNQFDPWGQGTLVTVSS |
| | | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPEAAGG |
| | | PSVFLFPPKPKDTLMISRTPEVTCVVVDVSHEDPEVKENWYVDGVEVHNAKTKPREEQYN |
| | | STYRVVSVLTVLHQDWLNGKEYKCAVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 89 | Anti-B7H4 | QLQLQESGPGLVKPSETLSLTCTVSGGSIKSGSYYWGWIRQPPGKGLEWIGNIYYSGSTY |
| | LALAKA HC | YNPSLRSRVTISVDTSKNQFSLKLSSVTAADTAVYYCAREGSYPNQFDPWGQGTLVTVSS |
| | v2 | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPEAAGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCAVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPG |
| 90 | Anti-B7H4 | EIVMTQSPATLSVSPGERATLSCRASQSVSSNLAWYQQKPGQAPRLLIYGASTRATGIPA |
| | LALAKA LC | RFSGSGSGTEFTLTISSLQSEDFAVYYCQQYHSFPFTFGGGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSENRGEC |
| 91 | Anti-B7H4 | GSISSSSYYWG |
| | CDR-H1 | |
| 92 | Anti-B7H4 | NIYYSGSTYYNPSLKS |
| | CDR-H2 | |
| 93 | Anti-B7H4 | AREGSYPNWFDP |
| | CDR-H3 | |
| 94 | Anti-B7H4 | RASQSVSSNLA |
| | CDR-L1 | |
| 95 | Anti-B7H4 | GASTRAT |
| | CDR-L2 | |
| 96 | Anti-B7H4 | QQYHSFPFT |
| | CDR-L3 | |
| 97 | Anti-B7H4 | QLQLQESGPGLVKPSETLSLTCTVSGGSISSSSYYWGWIRQPPGKGLEWIGNIYYSGSTY |
| | VH | YNPSLKSRVTISVDTSKNQFSLKLSSVTAADTAVYYCAREGSYPNWEDPWGQGTLVTVSS |
| 98 | Anti-B7H4 | EIVMTQSPATLSVSPGERATLSCRASQSVSSNLAWYQQKPGQAPRLLIYGASTRATGIPA |
| | VL | RFSGSGSGTEFTLTISSLQSEDFAVYYCQQYHSFPFTFGGGTKVEIK |
| 99 | Anti-B7H4 | GSIKSGSHYWG |
| | CDR-H1 | |
| 100 | Anti-B7H4 | NIYYSGSTYYNPSLRS |
| | CDR-H2 | |
| 101 | Anti-B7H4 | AREGSYPNWFDP |
| | CDR-H3 | |
| 102 | Anti-B7H4 | RASQSVSSNLA |
| | CDR-L1 | |
| 103 | Anti-B7H4 | GASTRAT |
| | CDR-L2 | |
| 104 | Anti-B7H4 | QQYHSFPFT |
| | CDR-L3 | |
| 105 | Anti-B7H4 | QLQLQESGPGLVKPSETLSLTCTVSGGSIKSGSHYWGWIRQPPGKGLEWIGNIYYSGSTY |
| | VH | YNPSLRSRVTISVDTSKNQFSLKLSSVTAADTAVYYCAREGSYPNWFDPWGQGTLVTVSS |
| 106 | Anti-B7H4 | EIVMTQSPATLSVSPGERATLSCRASQSVSSNLAWYQQKPGQAPRLLIYGASTRATGIPA |
| | VL | RFSGSGSGTEFTLTISSLQSEDFAVYYCQQYHSFPFTFGGGTKVEIK |
| 107 | Anti-B7H4 | GSIKSGSHYWG |
| | CDR-H1 | |
| 108 | Anti-B7H4 | NIYYSGSTYYNPSLKS |
| | CDR-H2 | |
| 109 | Anti-B7H4 | AREGSYPNWLDP |
| | CDR-H3 | |
| 110 | Anti-B7H4 | RASQSVSSNLA |
| | CDR-L1 | |
| 111 | Anti-B7H4 | GASTRAT |
| | CDR-L2 | |
| 112 | Anti-B7H4 | QQYHSFPFT |
| | CDR-L3 | |
| 113 | Anti-B7H4 | QLQLQESGPGLVKPSETLSLTCTVSGGSIKSGSHYWGWIRQPPGKGLEWIGNIYYSGSTY |
| | VH | YNPSLKSRVTISVDTSKNQFSLKLSSVTAADTAVYYCAREGSYPNWLDPWGQGTLVTVSS |
| 114 | Anti-B7H4 | EIVMTQSPATLSVSPGERATLSCRASQSVSSNLAWYQQKPGQAPRLLIYGASTRATGIPA |
| | VL | RFSGSGSGTEFTLTISSLQSEDFAVYYCQQYHSFPFTFGGGTKVEIK |
| 115 | Anti-B7H4 | GSIKSGSYYWG |
| | CDR-H1 | |
| 116 | Anti-B7H4 | NIYYSGSTYYNPSLKS |
| | CDR-H2 | |
| 117 | Anti-B7H4 | AREGSYPNQFDP |
| | CDR-H3 | |
| 118 | Anti-B7H4 | RASQSVSSNLA |
| | CDR-L1 | |
| 119 | Anti-B7H4 | GASTRAT |
| | CDR-L2 | |
| 120 | Anti-B7H4 | QQYHSFPFT |
| | CDR-L3 | |
| 121 | Anti-B7H4 | QLQLQESGPGLVKPSETLSLTCTVSGGSIKSGSYYWGWIRQPPGKGLEWIGNIYYSGSTY |
| | VH | YNPSLKSRVTISVDTSKNQFSLKLSSVTAADTAVYYCAREGSYPNQFDPWGQGILVTVSS |
| 122 | Anti-B7H4 | EIVMTQSPATLSVSPGERATLSCRASQSVSSNLAWYQQKPGQAPRLLIYGASTRATGIPA |
| | VL | RFSGSGSGTEFTLTISSLQSEDFAVYYCQQYHSFPFTFGGGTKVEIK |
| 123 | Anti-B7H4 | GSIKSGSHYWG |
| | CDR-H1 | |
| 124 | Anti-B7H4 | NIYYSGSTYYNPSLKS |
| | CDR-H2 | |
| 125 | Anti-B7H4 | AREGSYPNWFDP |
| | CDR-H3 | |
| 126 | Anti-B7H4 | RASQSVSTNLA |
| | CDR-L1 | |
| 127 | Anti-B7H4 | DASARVT |
| | CDR-L2 | |
| 128 | Anti-B7H4 | QQYHSFPFT |
| | CDR-L3 | |
| 129 | Anti-B7H4 | QLQLQESGPGLVKPSETLSLTCTVSGGSIKSGSHYWGWIRQPPGKGLEWIGNIYYSGSTY |
| | VH | YNPSLKSRVTMSVDTSKNQFSLKLSSVTAADTAVYYCAREGSYPNWFDPWGQGTLVTVSS |
| 130 | Anti-B7H4 | EIVMTQSPATLSVSPGERATLSCRASQSVSTNLAWYQQKPGQAPRLLIYDASARVTGIPA |
| | VL | RFSGSGSGTEFTLTISSLQSEDFAVYYCQQYHSFPFTFGGGTKVEIK |
| 131 | Anti-B7H4 | GSISSSSYYWG |
| | CDR-H1 | |
| 132 | Anti-B7H4 | NIYYSGSTYYNPSLKS |
| | CDR-H2 | |
| 133 | Anti-B7H4 | AREGSYTTVLNV |
| | CDR-H3 | |
| 134 | Anti-B7H4 | RASQSVSSSYLA |
| | CDR-L1 | |
| 135 | Anti-B7H4 | GASSRAT |
| | CDR-L2 | |
| 136 | Anti-B7H4 | QQAASYPLT |
| | CDR-L3 | |
| 137 | Anti-B7H4 | QLQLQESGPGLVKPSETLSLTCTVSGGSISSSSYYWGWIRQPPGKGLEWIGNIYYSGSTY |
| | VH | YNPSLKSRVTISVDTSKNQFSLKLSSVTAADTAVYYCAREGSYTTVLNVWGQGTMVTVSS |
| 138 | Anti-B7H4 | EIVLTQSPGTLSLSPGERATLSCRASQSVSSSYLAWYQQKPGQAPRLLIYGASSRATGIP |
| | VL | DRFSGSGSGTDFTLTISRLEPEDFAVYYCQQAASYPLTFGGGTKVEIK |
| 139 | Anti-B7H4 | GSIGRGSYYWG |
| | CDR-H1 | |
| 140 | Anti-B7H4 | NIYYSGSTYYNPSLKS |
| | CDR-H2 | |
| 141 | Anti-B7H4 | AREGSYTTVLNV |
| | CDR-H3 | |
| 142 | Anti-B7H4 | RASQSVASSHLA |
| | CDR-L1 | |
| 143 | Anti-B7H4 | DAVSRAT |
| | CDR-L2 | |
| 144 | Anti-B7H4 | QQAASYPLT |
| | CDR-L3 | |
| 145 | Anti-B7H4 | QLQLQESGPGLVKPSETLSLTCTVSGGSIGRGSYYWGWIRQPPGKGLEWIGNIYYSGSTY |
| | VH | YNPSLKSRVTISVDTSKNQFSLKLSSVTAADTAVYYCAREGSYTTVLNVWGQGTMVTVSS |
| 146 | Anti-B7H4 | EIVLTQSPGTLSLSPGERATLSCRASQSVASSHLAWYQQKPGQAPRLLIYDAVSRATGIP |
| | VL | DRFSGSGSGTDFTLTISRLEPEDFAVYYCQQAASYPLTFGGGTKVEIK |
| 147 | Anti-B7H4 | GSISSGGYYWS |
| | CDR-H1 | |
| 148 | Anti-B7H4 | NIYYSGSTYYNPSLKS |
| | CDR-H2 | |
| 149 | Anti-B7H4 | ARESSTISADFDL |
| | CDR-H3 | |
| 150 | Anti-B7H4 | RASQGISRWLA |
| | CDR-L1 | |
| 151 | Anti-B7H4 | AASSLQS |
| | CDR-L2 | |
| 152 | Anti-B7H4 | QQAHTFPYT |
| | CDR-L3 | |
| 153 | Anti-B7H4 | QVQLQESGPGLVKPSQTLSLTCTVSGGSISSGGYYWSWIRQHPGKGLEWIGNIYYSGSTY |
| | VH | YNPSLKSRVTISVDTSKNQFSLKLSSVTAADTAVYYCARESSTISADFDLWGRGTLVTVS |
| | | S |
| 154 | Anti-B7H4 | DIQMTQSPSSVSASVGDRVTITCRASQGISRWLAWYQQKPGKAPKLLIYAASSLQSGVPS |
| | VL | RFSGSGSGTDFTLTISSLQPEDFATYYCQQAHTFPYTFGGGTKVEIK |
| 155 | Anti-B7H4 | GSISHGGYYWS |
| | CDR-H1 | |
| 156 | Anti-B7H4 | NIYYSGSTYYNPSLKS |
| | CDR-H2 | |
| 157 | Anti-B7H4 | ARESSTISADFDL |
| | CDR-H3 | |
| 158 | Anti-B7H4 | RASQGISRWLA |
| | CDR-L1 | |
| 159 | Anti-B7H4 | AASSLQS |
| | CDR-L2 | |
| 160 | Anti-B7H4 | QQAHTFPYT |
| | CDR-L3 | |
| 161 | Anti-B7H4 | QLQLQESGPGLVKPSETLSLTCTASGGSISHGGYYWSWIRQHPGKGLEWIGNIYYSGSTY |
| | VH | YNPSLKSRVTMSVDTSKNQFSLKLSSVTAADTAVYYCARESSTISADFDLWGRGTLVTVS |
| | | S |
| 162 | Anti-B7H4 | DIQMTQSPSSVSASVGDRVTITCRASQGISRWLAWYQQKPGKAPKLLIYAASSLQSGVPS |
| | VL | RFSGSGSGTDFTLTISSLQPEDFATYYCQQAHTFPYTFGGGTKVEIK |
| 163 | Anti-B7H4 | GSISSGGYYWS |
| | CDR-H1 | |
| 164 | Anti-B7H4 | NIYYSGSTYYNPSLKS |
| | CDR-H2 | |
| 165 | Anti-B7H4 | ARGLSTIDEAFDP |
| | CDR-H3 | |
| 166 | Anti-B7H4 | RASQSISSWLA |
| | CDR-L1 | |
| 167 | Anti-B7H4 | KASSLES |
| | CDR-L2 | |
| 168 | Anti-B7H4 | QQDNSYPYT |
| | CDR-L3 | |
| 169 | Anti-B7H4 | QVQLQESGPGLVKPSQTLSLTCTVSGGSISSGGYYWSWIRQHPGKGLEWIGNIYYSGSTY |
| | VH | YNPSLKSRVTISVDTSKNQFSLKLSSVTAADTAVYYCARGLSTIDEAFDPWGQGTLVTVS |
| | | S |
| 170 | Anti-B7H4 | DIQMTQSPSTLSASVGDRVTITCRASQSISSWLAWYQQKPGKAPKLLIYKASSLESGVPS |
| | VL | RFSGSGSGTEFTLTISSLQPDDFATYYCQQDNSYPYTFGGGTKVEIK |
| 171 | Anti-B7H4 | GSISDGSYYWS |
| | CDR-H1 | |
| 172 | Anti-B7H4 | NIYYSGSTYYNPSLRS |
| | CDR-H2 | |
| 173 | Anti-B7H4 | ARGLSTIDEAFDP |
| | CDR-H3 | |
| 174 | Anti-B7H4 | RASQSISSWLA |
| | CDR-L1 | |
| 175 | Anti-B7H4 | KASSLES |
| | CDR-L2 | |
| 176 | Anti-B7H4 | QQDNSYPYT |
| | CDR-L3 | |
| 177 | Anti-B7H4 | QLQLQESGPGLVKPSETLSLTCTVSGGSISDGSYYWSWIRQHPGKGLEWIGNIYYSGSTY |
| | VH | YNPSLRSRVTMSVDTSKNQFSLKLSSVTAADTAVYYCARGLSTIDEAFDPWGQGTLVTVS |
| | | S |
| 178 | Anti-B7H4 | DIQMTQSPSTLSASVGDRVTITCRASQSISSWLAWYQQKPGKAPKLLIYKASSLESGVPS |
| | VL | RFSGSGSGTEFTLTISSLQPDDFATYYCQQDNSYPYTFGGGTKVEIK |
| 179 | Anti-B7H4 | GSISDGSYYWS |
| | CDR-H1 | |
| 180 | Anti-B7H4 | NIYYSGSTYYNPSLRS |
| | CDR-H2 | |
| 181 | Anti-B7H4 | ARGLSTIDEAFDP |
| | CDR-H3 | |
| 182 | Anti-B7H4 | RASKSISSWLA |
| | CDR-L1 | |
| 183 | Anti-B7H4 | EASSLHS |
| | CDR-L2 | |
| 184 | Anti-B7H4 | QQDNSYPYT |
| | CDR-L3 | |
| 185 | Anti-B7H4 | QVQLQESGPGLVKPSQTLSLTCTVSGGSISDGSYYWSWIRQHPGKGLEWIGNIYYSGSTY |
| | VH | YNPSLRSRVTMSVDTSKNQFSLKLSSVTAADTAVYYCARGLSTIDEAFDPWGQGTLVTVS |
| | | S |
| 186 | Anti-B7H4 | DIQMTQSPSTLSASVGDRVTITCRASKSISSWLAWYQQKPGKAPKLLIYEASSLHSGVPS |
| | VL | RFSGSGSGTEFTLTISSLQPDDFATYYCQQDNSYPYTFGGGTKVEIK |
| 187 | Anti-B7H4 | GSISSYYWS |
| | CDR-H1 | |
| 188 | Anti-B7H4 | YIYSSGSTNYNPSLKS |
| | CDR-H2 | |
| 189 | Anti-B7H4 | ARGSGQYAAPDYGMD |
| | CDR-H3 | |
| 190 | Anti-B7H4 | RASQSISSWLA |
| | CDR-L1 | |
| 191 | Anti-B7H4 | KASSLES |
| | CDR-L2 | |
| 192 | Anti-B7H4 | QQDNSFPFT |
| | CDR-L3 | |
| 193 | Anti-B7H4 | QVQLQESGPGLVKPSETLSLTCTVSGGSISSYYWSWIRQPPGKGLEWIGYIYSSGSTNYN |
| | VH | PSLKSRVTISVDTSKNQFSLKLSSVTAADTAVYYCARGSGQYAAPDYGMDVWGQGTTVTV |
| | | SS |
| 194 | Anti-B7H4 | DIQMTQSPSTLSASVGDRVTITCRASQSISSWLAWYQQKPGKAPKLLIYKASSLESGVPS |
| | VL | RFSGSGSGTEFTLTISSLQPDDFATYYCQQDNSFPFTFGGGTKVEIK |
| 195 | Anti-B7H4 | GSIISYYWG |
| | CDR-H1 | |
| 196 | Anti-B7H4 | YIYSSGSTSYNPSLKS |
| | CDR-H2 | |
| 197 | Anti-B7H4 | ARGSGLYAAPDYGLDV |
| | CDR-H3 | |
| 198 | Anti-B7H4 | RASQSISSWLA |
| | CDR-L1 | |
| 199 | Anti-B7H4 | KASSLES |
| | CDR-L2 | |
| 200 | Anti-B7H4 | QQDNSFPFT |
| | CDR-L3 | |
| 201 | Anti-B7H4 | QVQLQESGPGLVKPSETLSLTCTVSGGSIISYYWGWIRQPPGKGLEWIGYIYSSGSTSYN |
| | VH | PSLKSRVTISVDTSKNQFSLKLSSVTAADTAVYYCARGSGLYAAPDYGLDVWGQGTTVTV |
| | | SS |
| 202 | Anti-B7H4 | DIQMTQSPSTLSASVGDRVTITCRASQSISSWLAWYQQKPGKAPKLLIYKASSLESGVPS |
| | VL | RFSGSGSGTEFTLTISSLQPDDFATYYCQQDNSFPFTFGGGTKVEIK |
| 203 | Anti-B7H4 | FTFSSYAMS |
| | CDR-H1 | |
| 204 | Anti-B7H4 | TISGSGGSTYYADSVKG |
| | CDR-H2 | |
| 205 | Anti-B7H4 | ARGAGHYDLVGRY |
| | CDR-H3 | |
| 206 | Anti-B7H4 | RASQSISSYLN |
| | CDR-L1 | |
| 207 | Anti-B7H4 | AASSLQS |
| | CDR-L2 | |
| 208 | Anti-B7H4 | QQLYSLPPT |
| | CDR-L3 | |
| 209 | Anti-B7H4 | EVQLLESGGGLVQPGGSLRLSCAASGFTFSSYAMSWVRQAPGKGLEWVSTISGSGGSTYY |
| | VH | ADSVKGRFTISRDNSKNTLYLQMNSLRAEDTAVYYCARGAGHYDLVGRYWGQGTLVTVSS |
| 210 | Anti-B7H4 | DIQMTQSPSSLSASVGDRVTITCRASQSISSYLNWYQQKPGKAPKLLIYAASSLQSGVPS |
| | VL | RFSGSGSGTDFTLTISSLQPEDFATYYCQQLYSLPPTFGGGTKVEIK |
| 211 | Anti-B7H4 | FTFSSYAMS |
| | CDR-H1 | |
| 212 | Anti-B7H4 | AISGSGGSTYYADSVKG |
| | CDR-H2 | |
| 213 | Anti-B7H4 | ARVGFRALNY |
| | CDR-H3 | |
| 214 | Anti-B7H4 | RASQDISSWLA |
| | CDR-L1 | |
| 215 | Anti-B7H4 | AASSLQS |
| | CDR-L2 | |
| 216 | Anti-B7H4 | QQATSYPPWT |
| | CDR-L3 | |
| 217 | Anti-B7H4 | EVQLLESGGGLVQPGGSLRLSCAASGFTFSSYAMSWVRQAPGKGLEWVSAISGSGGSTYY |
| | VH | ADSVKGRFTISRDNSKNTLYLQMNSLRAEDTAVYYCARVGFRALNYWGQGTTVTVSS |
| 218 | Anti-B7H4 | DIQLTQSPSSVSASVGDRVTITCRASQDISSWLAWYQQKPGKAPKLLIYAASSLQSGVPS |
| | VL | RFSGSGSGTDFTLTISSLQPEDFATYYCQQATSYPPWTFGGGTKVEIK |
| 219 | Anti-B7H4 | GTFSSYAIS |
| | CDR-H1 | |
| 220 | Anti-B7H4 | GIIPIFGTASYAQKFQG |
| | CDR-H2 | |
| 221 | Anti-B7H4 | ARQQYDGRRYFGL |
| | CDR-H3 | |
| 222 | Anti-B7H4 | RASQSVSSNLA |
| | CDR-L1 | |
| 223 | Anti-B7H4 | SASTRAT |
| | CDR-L2 | |
| 224 | Anti-B7H4 | QQVNVWPPT |
| | CDR-L3 | |
| 225 | Anti-B7H4 | QVQLVQSGAEVKKPGSSVKVSCKASGGTFSSYAISWVRQAPGQGLEWMGGIIPIFGTASY |
| | VH | AQKFQGRVTITADESTSTAYMELSSLRSEDTAVYYCARQQYDGRRYFGLWGRGTLVTVSS |
| 226 | Anti-B7H4 | EIVMTQSPATLSVSPGERATLSCRASQSVSSNLAWYQQKPGQAPRLLIYSASTRATGIPA |
| | VL | RFSGSGSGTEFTLTISSLQSEDFAVYYCQQVNVWPPTFGGGTKVEIK |
| 227 | Anti-B7H4 | GTFSSYAIS |
| | CDR-H1 | |
| 228 | Anti-B7H4 | GIIPIFGTANYAQKFQG |
| | CDR-H2 | |
| 229 | Anti-B7H4 | ARGGPWFDP |
| | CDR-H3 | |
| 230 | Anti-B7H4 | RASQSISSWLA |
| | CDR-L1 | |
| 231 | Anti-B7H4 | KASSLES |
| | CDR-L2 | |
| 232 | Anti-B7H4 | QQYNSYPPFT |
| | CDR-L3 | |
| 233 | Anti-B7H4 | QVQLVQSGAEVKKPGSSVKVSCKASGGTFSSYAISWVRQAPGQGLEWMGGIIPIFGTANY |
| | VH | AQKFQGRVTITADESTSTAYMELSSLRSEDTAVYYCARGGPWFDPWGQGTLVTVSS |
| 234 | Anti-B7H4 | DIQMTQSPSTLSASVGDRVTITCRASQSISSWLAWYQQKPGKAPKLLIYKASSLESGVPS |
| | VL | RFSGSGSGTEFTLTISSLQPDDFATYYCQQYNSYPPFTFGGGTKVEIK |
| 235 | Anti-B7H4 | FTFSSYAMS |
| | CDR-H1 | |
| 236 | Anti-B7H4 | AISGSGGSTSYADSVKG |
| | CDR-H2 | |
| 237 | Anti-B7H4 | AKPSLATMLAFDI |
| | CDR-H3 | |
| 238 | Anti-B7H4 | RASQSISSWLA |
| | CDR-L1 | |
| 239 | Anti-B7H4 | DASSLES |
| | CDR-L2 | |
| 240 | Anti-B7H4 | QQSKSYPRT |
| | CDR-L3 | |
| 241 | Anti-B7H4 | EVQLLESGGGLVQPGGSLRLSCAASGFTFSSYAMSWVRQAPGKGLEWVSAISGSGGSTSY |
| | VH | ADSVKGRFTISRDNSKNTLYLQMNSLRAEDTAVYYCAKPSLATMLAFDIWGQGTMVTVSS |
| 242 | Anti-B7H4 | DIQMTQSPSTLSASVGDRVTITCRASQSISSWLAWYQQKPGKAPKLLIYDASSLESGVPS |
| | VL | RFSGSGSGTEFTLTISSLQPDDFATYYCQQSKSYPRTFGGGTKVEIK |
| 243 | Anti-B7H4 | GSISSSVYYWS |
| | CDR-H1 | |
| 244 | Anti-B7H4 | SILVSGSTYYNPSLKS |
| | CDR-H2 | |
| 245 | Anti-B7H4 | ARAVSFLDV |
| | CDR-H3 | |
| 246 | Anti-B7H4 | RASQSISSYLN |
| | CDR-L1 | |
| 247 | Anti-B7H4 | GASSLQS |
| | CDR-L2 | |
| 248 | Anti-B7H4 | QQSYDPPWT |
| | CDR-L3 | |
| 249 | Anti-B7H4 | QLQLQESGPGLVKPSETLSLTCTVSGGSISSSVYYWSWIRQPPGKGLEWIGSILVSGSTY |
| | VH | YNPSLKSRVTISVDTSKNQFSLKLSSVTAADTAVYYCARAVSFLDVWGQGTMVIVSS |
| 250 | Anti-B7H4 | DIQMTQSPSSLSASVGDRVTITCRASQSISSYLNWYQQKPGKAPKLLIYGASSLQSGVPS |
| | VL | RFSGSGSGTDFTLTISSLQPEDFATYYCQQSYDPPWTFGGGTKVEIK |
| 251 | Anti-B7H4 | QLQLQESGPGLVKPSETLSLTCTVSGGSISSSSYYWGWIRQPPGKGLEWIGNIYYSGSTY |
| | HC | YNPSLKSRVTISVDTSKNQFSLKLSSVTAADTAVYYCAREGSYPNWFDPWGQGTLVTVSS |
| | | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 252 | Anti-B7H4 | EIVMTQSPATLSVSPGERATLSCRASQSVSSNLAWYQQKPGQAPRLLIYGASTRATGIPA |
| | LC | RFSGSGSGTEFTLTISSLQSEDFAVYYCQQYHSFPFTFGGGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSENRGEC |
| 253 | Anti-B7H4 | QLQLQESGPGLVKPSETLSLTCTVSGGSIKSGSHYWGWIRQPPGKGLEWIGNIYYSGSTY |
| | HC | YNPSLRSRVTISVDTSKNQFSLKLSSVTAADTAVYYCAREGSYPNWFDPWGQGTLVTVSS |
| | | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 254 | Anti-B7H4 | EIVMTQSPATLSVSPGERATLSCRASQSVSSNLAWYQQKPGQAPRLLIYGASTRATGIPA |
| | LC | RFSGSGSGTEFTLTISSLQSEDFAVYYCQQYHSFPFTFGGGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSENRGEC |
| 255 | Anti-B7H4 | QLQLQESGPGLVKPSETLSLTCTVSGGSIKSGSHYWGWIRQPPGKGLEWIGNIYYSGSTY |
| | HC | YNPSLKSRVTISVDTSKNQFSLKLSSVTAADTAVYYCAREGSYPNWLDPWGQGTLVTVSS |
| | | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKENWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 256 | Anti-B7H4 | EIVMTQSPATLSVSPGERATLSCRASQSVSSNLAWYQQKPGQAPRLLIYGASTRATGIPA |
| | LC | RFSGSGSGTEFTLTISSLQSEDFAVYYCQQYHSFPFTFGGGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSENRGEC |
| 257 | Anti-B7H4 | QLQLQESGPGLVKPSETLSLTCTVSGGSIKSGSYYWGWIRQPPGKGLEWIGNIYYSGSTY |
| | HC | YNPSLKSRVTISVDTSKNQFSLKLSSVTAADTAVYYCAREGSYPNQFDPWGQGILVTVSS |
| | | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 258 | Anti-B7H4 | EIVMTQSPATLSVSPGERATLSCRASQSVSSNLAWYQQKPGQAPRLLIYGASTRATGIPA |
| | LC | RFSGSGSGTEFTLTISSLQSEDFAVYYCQQYHSFPFTFGGGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 259 | Anti-B7H4 | QLQLQESGPGLVKPSETLSLTCTVSGGSIKSGSHYWGWIRQPPGKGLEWIGNIYYSGSTY |
| | HC | YNPSLKSRVTMSVDTSKNQFSLKLSSVTAADTAVYYCAREGSYPNWFDPWGQGTLVTVSS |
| | | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 260 | Anti-B7H4 | EIVMTQSPATLSVSPGERATLSCRASQSVSTNLAWYQQKPGQAPRLLIYDASARVTGIPA |
| | LC | RFSGSGSGTEFTLTISSLQSEDFAVYYCQQYHSFPFTFGGGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSFNRGEC |
| 261 | Anti-B7H4 | QLQLQESGPGLVKPSETLSLTCTVSGGSISSSSYYWGWIRQPPGKGLEWIGNIYYSGSTY |
| | HC | YNPSLKSRVTISVDTSKNQFSLKLSSVTAADTAVYYCAREGSYTTVLNVWGQGTMVTVSS |
| | | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 262 | Anti-B7H4 | EIVLTQSPGTLSLSPGERATLSCRASQSVSSSYLAWYQQKPGQAPRLLIYGASSRATGIP |
| | LC | DRFSGSGSGTDFTLTISRLEPEDFAVYYCQQAASYPLTFGGGTKVEIKRTVAAPSVFIFP |
| | | PSDEQLKSGTASVVCLLNNFYPREAKVQWKVDNALQSGNSQESVTEQDSKDSTYSLSSTL |
| | | TLSKADYEKHKVYACEVTHQGLSSPVTKSENRGEC |
| 263 | Anti-B7H4 | QLQLQESGPGLVKPSETLSLTCTVSGGSIGRGSYYWGWIRQPPGKGLEWIGNIYYSGSTY |
| | HC | YNPSLKSRVTISVDTSKNQFSLKLSSVTAADTAVYYCAREGSYTTVLNVWGQGTMVTVSS |
| | | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 264 | Anti-B7H4 | EIVLTQSPGTLSLSPGERATLSCRASQSVASSHLAWYQQKPGQAPRLLIYDAVSRATGIP |
| | LC | DRFSGSGSGTDFTLTISRLEPEDFAVYYCQQAASYPLTFGGGTKVEIKRTVAAPSVFIFP |
| | | PSDEQLKSGTASVVCLLNNFYPREAKVQWKVDNALQSGNSQESVTEQDSKDSTYSLSSTL |
| | | TLSKADYEKHKVYACEVTHQGLSSPVTKSENRGEC |
| 265 | Anti-B7H4 | QVQLQESGPGLVKPSQTLSLTCTVSGGSISSGGYYWSWIRQHPGKGLEWIGNIYYSGSTY |
| | HC | YNPSLKSRVTISVDTSKNQFSLKLSSVTAADTAVYYCARESSTISADFDLWGRGTLVTVS |
| | | SASTKGPSVFPLAPSSKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDKTHTCPPCPAPELLG |
| | | GPSVFLFPPKPKDTLMISRTPEVTCVVVDVSHEDPEVKENWYVDGVEVHNAKTKPREEQY |
| | | NSTYRVVSVLTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLYSKLTVDKSR |
| | | WQQGNVFSCSVMHEALHNHYTQKSLSLSPGK |
| 266 | Anti-B7H4 | DIQMTQSPSSVSASVGDRVTITCRASQGISRWLAWYQQKPGKAPKLLIYAASSLQSGVPS |
| | LC | RFSGSGSGTDFTLTISSLQPEDFATYYCQQAHTFPYTFGGGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSENRGEC |
| 267 | Anti-B7H4 | QLQLQESGPGLVKPSETLSLTCTASGGSISHGGYYWSWIRQHPGKGLEWIGNIYYSGSTY |
| | HC | YNPSLKSRVTMSVDTSKNQFSLKLSSVTAADTAVYYCARESSTISADFDLWGRGTLVTVS |
| | | SASTKGPSVFPLAPSSKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLYSKLTVDKSR |
| | | WQQGNVFSCSVMHEALHNHYTQKSLSLSPGK |
| 268 | Anti-B7H4 | DIQMTQSPSSVSASVGDRVTITCRASQGISRWLAWYQQKPGKAPKLLIYAASSLQSGVPS |
| | LC | RFSGSGSGTDFTLTISSLQPEDFATYYCQQAHTFPYTFGGGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSENRGEC |
| 269 | Anti-B7H4 | QVQLQESGPGLVKPSQTLSLTCTVSGGSISSGGYYWSWIRQHPGKGLEWIGNIYYSGSTY |
| | HC | YNPSLKSRVTISVDTSKNQFSLKLSSVTAADTAVYYCARGLSTIDEAFDPWGQGTLVTVS |
| | | SASTKGPSVFPLAPSSKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLYSKLTVDKSR |
| | | WQQGNVFSCSVMHEALHNHYTQKSLSLSPGK |
| 270 | Anti-B7H4 | DIQMTQSPSTLSASVGDRVTITCRASQSISSWLAWYQQKPGKAPKLLIYKASSLESGVPS |
| | LC | RFSGSGSGTEFTLTISSLQPDDFATYYCQQDNSYPYTFGGGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSENRGEC |
| 271 | Anti-B7H4 | QLQLQESGPGLVKPSETLSLTCTVSGGSISDGSYYWSWIRQHPGKGLEWIGNIYYSGSTY |
| | HC | YNPSLRSRVTMSVDTSKNQFSLKLSSVTAADTAVYYCARGLSTIDEAFDPWGQGTLVTVS |
| | | SASTKGPSVFPLAPSSKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLYSKLTVDKSR |
| | | WQQGNVFSCSVMHEALHNHYTQKSLSLSPGK |
| 272 | Anti-B7H4 | DIQMTQSPSTLSASVGDRVTITCRASQSISSWLAWYQQKPGKAPKLLIYKASSLESGVPS |
| | LC | RFSGSGSGTEFTLTISSLQPDDFATYYCQQDNSYPYTFGGGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSENRGEC |
| 273 | Anti-B7H4 | QVQLQESGPGLVKPSQTLSLTCTVSGGSISDGSYYWSWIRQHPGKGLEWIGNIYYSGSTY |
| | HC | YNPSLRSRVTMSVDTSKNQFSLKLSSVTAADTAVYYCARGLSTIDEAFDPWGQGTLVTVS |
| | | SASTKGPSVFPLAPSSKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREPQVYTLPPSRD |
| | | ELTKNQVSLTCLVKGFYPSDIAVEWESNGQPENNYKTTPPVLDSDGSFFLYSKLTVDKSR |
| | | WQQGNVFSCSVMHEALHNHYTQKSLSLSPGK |
| 274 | Anti-B7H4 | DIQMTQSPSTLSASVGDRVTITCRASKSISSWLAWYQQKPGKAPKLLIYEASSLHSGVPS |
| | LC | RFSGSGSGTEFTLTISSLQPDDFATYYCQQDNSYPYTFGGGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSENRGEC |
| 275 | Anti-B7H4 | QVQLQESGPGLVKPSETLSLTCTVSGGSISSYYWSWIRQPPGKGLEWIGYIYSSGSTNYN |
| | HC | PSLKSRVTISVDTSKNQFSLKLSSVTAADTAVYYCARGSGQYAAPDYGMDVWGQGTTVTV |
| | | SSASTKGPSVFPLAPSSKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSCDKTHTCPPCPAPELL |
| | | GGPSVFLFPPKPKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPREPQVYTLPPSR |
| | | DELTKNQVSLTCLVKGFYPSDIAVEWESNGQPENNYKTTPPVLDSDGSFFLYSKLTVDKS |
| | | RWQQGNVFSCSVMHEALHNHYTQKSLSLSPGK |
| 276 | Anti-B7H4 | DIQMTQSPSTLSASVGDRVTITCRASQSISSWLAWYQQKPGKAPKLLIYKASSLESGVPS |
| | LC | RFSGSGSGTEFTLTISSLQPDDFATYYCQQDNSFPFTFGGGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSENRGEC |
| 277 | Anti-B7H4 | QVQLQESGPGLVKPSETLSLTCTVSGGSIISYYWGWIRQPPGKGLEWIGYIYSSGSTSYN |
| | HC | PSLKSRVTISVDTSKNQFSLKLSSVTAADTAVYYCARGSGLYAAPDYGLDVWGQGTTVTV |
| | | SSASTKGPSVFPLAPSSKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSCDKTHTCPPCPAPELL |
| | | GGPSVFLFPPKPKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPREPQVYTLPPSR |
| | | DELTKNQVSLTCLVKGFYPSDIAVEWESNGQPENNYKTTPPVLDSDGSFFLYSKLTVDKS |
| | | RWQQGNVFSCSVMHEALHNHYTQKSLSLSPGK |
| 278 | Anti-B7H4 | DIQMTQSPSTLSASVGDRVTITCRASQSISSWLAWYQQKPGKAPKLLIYKASSLESGVPS |
| | LC | RFSGSGSGTEFTLTISSLQPDDFATYYCQQDNSFPFTFGGGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSENRGEC |
| 279 | Anti-B7H4 | EVQLLESGGGLVQPGGSLRLSCAASGFTFSSYAMSWVRQAPGKGLEWVSTISGSGGSTYY |
| | HC | ADSVKGRFTISRDNSKNTLYLQMNSLRAEDTAVYYCARGAGHYDLVGRYWGQGTLVTVSS |
| | | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 280 | Anti-B7H4 | DIQMTQSPSSLSASVGDRVTITCRASQSISSYLNWYQQKPGKAPKLLIYAASSLQSGVPS |
| | LC | RFSGSGSGTDFTLTISSLQPEDFATYYCQQLYSLPPTFGGGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSENRGEC |
| 281 | Anti-B7H4 | EVQLLESGGGLVQPGGSLRLSCAASGFTFSSYAMSWVRQAPGKGLEWVSAISGSGGSTYY |
| | HC | ADSVKGRFTISRDNSKNTLYLQMNSLRAEDTAVYYCARVGFRALNYWGQGTTVTVSSAST |
| | | KGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPCPAPELLGGPSV |
| | | FLFPPKPKDTLMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYTLPPSRDELTK |
| | | NQVSLTCLVKGFYPSDIAVEWESNGQPENNYKTTPPVLDSDGSFFLYSKLTVDKSRWQQG |
| | | NVFSCSVMHEALHNHYTQKSLSLSPGK |
| 282 | Anti-B7H4 | DIQLTQSPSSVSASVGDRVTITCRASQDISSWLAWYQQKPGKAPKLLIYAASSLQSGVPS |
| | LC | RFSGSGSGTDFTLTISSLQPEDFATYYCQQATSYPPWTFGGGTKVEIKRTVAAPSVFIFP |
| | | PSDEQLKSGTASVVCLLNNFYPREAKVQWKVDNALQSGNSQESVTEQDSKDSTYSLSSTL |
| | | TLSKADYEKHKVYACEVTHQGLSSPVTKSENRGEC |
| 283 | Anti-B7H4 | QVQLVQSGAEVKKPGSSVKVSCKASGGTFSSYAISWVRQAPGQGLEWMGGIIPIFGTASY |
| | HC | AQKFQGRVTITADESTSTAYMELSSLRSEDTAVYYCARQQYDGRRYFGLWGRGTLVTVSS |
| | | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 284 | Anti-B7H4 | EIVMTQSPATLSVSPGERATLSCRASQSVSSNLAWYQQKPGQAPRLLIYSASTRATGIPA |
| | LC | RFSGSGSGTEFTLTISSLQSEDFAVYYCQQVNVWPPTFGGGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSENRGEC |
| 285 | Anti-B7H4 | QVQLVQSGAEVKKPGSSVKVSCKASGGTFSSYAISWVRQAPGQGLEWMGGIIPIFGTANY |
| | HC | AQKFQGRVTITADESTSTAYMELSSLRSEDTAVYYCARGGPWFDPWGQGTLVTVSSASTK |
| | | GPSVFPLAPSSKSTSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPPCPAPELLGGPSVF |
| | | LFPPKPKDTLMISRTPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQVYTLPPSRDELTKN |
| | | QVSLTCLVKGFYPSDIAVEWESNGQPENNYKTTPPVLDSDGSFFLYSKLTVDKSRWQQGN |
| | | VFSCSVMHEALHNHYTQKSLSLSPGK |
| 286 | Anti-B7H4 | DIQMTQSPSTLSASVGDRVTITCRASQSISSWLAWYQQKPGKAPKLLIYKASSLESGVPS |
| | LC | RFSGSGSGTEFTLTISSLQPDDFATYYCQQYNSYPPFTFGGGTKVEIKRTVAAPSVFIFP |
| | | PSDEQLKSGTASVVCLLNNFYPREAKVQWKVDNALQSGNSQESVTEQDSKDSTYSLSSTL |
| | | TLSKADYEKHKVYACEVTHQGLSSPVTKSENRGEC |
| 287 | Anti-B7H4 | EVQLLESGGGLVQPGGSLRLSCAASGFTFSSYAMSWVRQAPGKGLEWVSAISGSGGSTSY |
| | HC | ADSVKGRFTISRDNSKNTLYLQMNSLRAEDTAVYYCAKPSLATMLAFDIWGQGTMVTVSS |
| | | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 288 | Anti-B7H4 | DIQMTQSPSTLSASVGDRVTITCRASQSISSWLAWYQQKPGKAPKLLIYDASSLESGVPS |
| | LC | RFSGSGSGTEFTLTISSLQPDDFATYYCQQSKSYPRTFGGGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSENRGEC |
| 289 | Anti-B7H4 | QLQLQESGPGLVKPSETLSLTCTVSGGSISSSVYYWSWIRQPPGKGLEWIGSILVSGSTY |
| | HC | YNPSLKSRVTISVDTSKNQFSLKLSSVTAADTAVYYCARAVSFLDVWGQGTMVIVSSAST |
| | | KGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPCPAPELLGGPSV |
| | | FLFPPKPKDTLMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYTLPPSRDELTK |
| | | NQVSLTCLVKGFYPSDIAVEWESNGQPENNYKTTPPVLDSDGSFFLYSKLTVDKSRWQQG |
| | | NVFSCSVMHEALHNHYTQKSLSLSPGK |
| 290 | Anti-B7H4 | DIQMTQSPSSLSASVGDRVTITCRASQSISSYLNWYQQKPGKAPKLLIYGASSLQSGVPS |
| | LC | RFSGSGSGTDFTLTISSLQPEDFATYYCQQSYDPPWTFGGGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSENRGEC |
| 291 | Anti-CD70 | NYGMN |
| | CDR-H1 | |
| 292 | Anti- | WINTYTGEPTYADAFKG |
| | CD70CDR-H2 | |
| 293 | Anti-CD70 | DYGDYGMDY |
| | CDR-H3 | |
| 294 | Anti-CD70 | RASKSVSTSGYSFMH |
| | CDR-L1 | |
| 295 | Anti-CD70 | LASNLES |
| | CDR-L2 | |
| 296 | Anti-CD70 | QHSREVPWT |
| | CDR-L3 | |
| 297 | Anti-CD70 | QVQLVQSGAEVKKPGASVKVSCKASGYTFTNYGMNWVRQAPGQGLKWMGWINTYTGEPTY |
| | VH | ADAFKGRVTMTRDTSISTAYMELSRLRSDDTAVYYCARDYGDYGMDYWGQGTTVTVSS |
| 298 | Anti-CD70 | DIVMTQSPDSLAVSLGERATINCRASKSVSTSGYSFMHWYQQKPGQPPKLLIYLASNLES |
| | VL | GVPDRFSGSGSGTDFTLTISSLQAEDVAVYYCQHSREVPWTFGQGTKVEIK |
| 299 | Anti-CD70 | QVQLVQSGAEVKKPGASVKVSCKASGYTFTNYGMNWVRQAPGQGLKWMGWINTYTGEPTY |
| | HC | ADAFKGRVTMTRDTSISTAYMELSRLRSDDTAVYYCARDYGDYGMDYWGQGTTVTVSSAS |
| | | TKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQVYTLPPSRDELT |
| | | KNQVSLTCLVKGFYPSDIAVEWESNGQPENNYKTTPPVLDSDGSFFLYSKLTVDKSRWQQ |
| | | GNVFSCSVMHEALHNHYTQKSLSLSPGK |
| 300 | Anti-CD70 | DIVMTQSPDSLAVSLGERATINCRASKSVSTSGYSFMHWYQQKPGQPPKLLIYLASNLES |
| | LC | GVPDRFSGSGSGTDFTLTISSLQAEDVAVYYCQHSREVPWTFGQGTKVEIKRTVAAPSVF |
| | | IFPPSDEQLKSGTASVVCLLNNFYPREAKVQWKVDNALQSGNSQESVTEQDSKDSTYSLS |
| | | STLTLSKADYEKHKVYACEVTHQGLSSPVTKSENRGEC |
| 301 | Anti-TROP2 | NYGMN |
| | CDR-H1 | |
| 302 | Anti-TROP2 | WINTYTGEPTYTDDFKG |
| | CDR-H2 | |
| 303 | Anti-TROP2 | GGFGSSYWYFDV |
| | CDR-H3 | |
| 304 | Anti-TROP2 | KASQDVSIAVA |
| | CDR-L1 | |
| 305 | Anti-TROP2 | SASYRYT |
| | CDR-L2 | |
| 306 | Anti-TROP2 | QQHYITPLT |
| | CDR-L3 | |
| 307 | Anti-TROP2 | QVQLQQSGSELKKPGASVKVSCKASGYTFTNYGMNWVKQAPGQGLKWMGWINTYTGEPTY |
| | VH | TDDFKGRFAFSLDTSVSTAYLQISSLKADDTAVYFCARGGFGSSYWYFDVWGQGSLVTVS |
| | | S |
| 308 | Anti-TROP2 | DIQLTQSPSSLSASVGDRVSITCKASQDVSIAVAWYQQKPGKAPKLLIYSASYRYTGVPD |
| | VL | RFSGSGSGTDFTLTISSLQPEDFAVYYCQQHYITPLTFGAGTKVEIK |
| 309 | Anti-TROP2 | TAGMQ |
| | CDR-H1 | |
| 310 | Anti-TROP2 | WINTHSGVPKYAEDFKG |
| | CDR-H2 | |
| 311 | Anti-TROP2 | SGFGSSYWYFDV |
| | CDR-H3 | |
| 312 | Anti-TROP2 | KASQDVSTAVA |
| | CDR-L1 | |
| 313 | Anti-TROP2 | SASYRYT |
| | CDR-L2 | |
| 314 | Anti-TROP2 | QQHYITPLT |
| | CDR-L3 | |
| 315 | Anti-TROP2 | QVQLVQSGAEVKKPGASVKVSCKASGYTFTTAGMQWVRQAPGQGLEWMGWINTHSGVPKY |
| | VH | AEDFKGRVTISADTSTSTAYLQLSSLKSEDTAVYYCARSGFGSSYWYFDVWGQGTLVTVS |
| | | S |
| 316 | Anti-TROP2 | DIQMTQSPSSLSASVGDRVTITCKASQDVSTAVAWYQQKPGKAPKLLIYSASYRYTGVPS |
| | VL | RFSGSGSGTDFTLTISSLQPEDFAVYYCQQHYITPLTFGQGTKLEIK |
| 317 | MICA CDR-H1 | SQNIY |
| 318 | MICA CDR-H2 | YIEPYNVVPMYNPKFKG |
| 319 | MICA CDR-H3 | SGSSNFDY |
| 320 | MICA CDR-L1 | SASSSISSHYLH |
| 321 | MICA CDR-L2 | RTSNLAS |
| 322 | MICA CDR-L3 | QQGSSLPLT |
| 323 | Anti-MICA | EIQLVQSGAEVKKPGASVKVSCKASGYAFTSQNIYWVRQAPGQGLEWIGYIEPYNVVPMY |
| | VH | NPKFKGRATLTVDKSTSTAYLELSSLRSEDTAVYYCARSGSSNFDYWGQGTLVTVSS |
| 324 | Anti-MICA | DIQLTQSPSSLSASVGDRVTITCSASSSISSHYLHWYQQKPGKSPKLLIYRTSNLASGVP |
| | VL | SRFSGSGSGTDYTLTISSLQPEDFATYYCQQGSSLPLTFGQGTKVEIK |
| 325 | Anti-MICA | NYAMH |
| | CDR-H1 | |
| 326 | Anti-MICA | LIWYDGSNKFYGDSVKG |
| | CDR-H2 | |
| 327 | Anti-MICA | EGSGHY |
| | CDR-H3 | |
| 328 | Anti-MICA | RASQGISSALA |
| | CDR-L1 | |
| 329 | Anti-MICA | DASSLES |
| | CDR-L2 | |
| 330 | Anti-MICA | QQFNSYPIT |
| | CDR-L3 | |
| 331 | Anti-MICA | QVQLVESGGGVVQPGRSLRLSCAASGFTFSNYAMHWVRQAPGEGLEWVALIWYDGSNKFY |
| | VH | GDSVKGRFTISRDNSKNTLYLQMNSLSAEDTAVYYCAREGSGHYWGQGTLVTVSS |
| 332 | Anti-MICA | AIQLTQSPSSLSASVGDRVTITCRASQGISSALAWYQQKPGKVPKSLIYDASSLESGVPS |
| | VL | RFSGSGSGTDFTLTISSLQPEDFATYYCQQFNSYPITFGQGTRLEIK |
| 333 | Anti-MICA | NYAMS |
| | CDR-H1 | |
| 334 | Anti-MICA | YISPGGDYIYYADSVKG |
| | CDR-H2 | |
| 335 | Anti-MICA | DRRHYGSYAMDY |
| | CDR-H3 | |
| 336 | Anti-MICA | RSSKSLLHSNLNTYLY |
| | CDR-L1 | |
| 337 | Anti-MICA | RMSNLAS |
| | CDR-L2 | |
| 338 | Anti-MICA | MQHLEYPFT |
| | CDR-L3 | |
| 339 | Anti-MICA | QVQLVESGGGLVKPGGSLRLSCAASGFTFSNYAMSWIRQAPGKGLEWVSYISPGGDYIYY |
| | VH | ADSVKGRFTISRDNAKNSLYLQMNSLRAEDTAVYYCTTDRRHYGSYAMDYWGQGTLVTVS |
| | | S |
| 340 | Anti-MICA | DIVMTQSPLSLPVTPGEPASISCRSSKSLLHSNLNTYLYWFLQKPGQSPQILIYRMSNLA |
| | VL | SGVPDRFSGSGSGTAFTLKISRVEAEDVGVYYCMQHLEYPFTFGPGTKLEIK |
| 341 | Anti-MICA | TYAFH |
| | CDR-H1 | |
| 342 | Anti-MICA | GIVPIFGTLKYAQKFQD |
| | CDR-H2 | |
| 343 | Anti-MICA | AIQLEGRPFDH |
| | CDR-H3 | |
| 344 | Anti-MICA | RASQGITSYLA |
| | CDR-L1 | |
| 345 | Anti-MICA | AASALQS |
| | CDR-L2 | |
| 346 | Anti-MICA | QQVNRGAAIT |
| | CDR-L3 | |
| 347 | Anti-MICA | QVQLVQSGAEVKKPGSSVRVSCRASGGSSTTYAFHWVRQAPGQGLEWMGGIVPIFGTLKY |
| | VH | AQKFQDRVTLTADKSTGTAYMELNSLRLDDTAVYYCARAIQLEGRPFDHWGQGTQVTVSA |
| 348 | Anti-MICA | DIQLTQSPSFLSASVGDRVTITCRASQGITSYLAWYQQKPGKAPKLLIYAASALQSGVPS |
| | VL | RFSGRGSGTEFTLTISSLQPEDFATYYCQQVNRGAAITFGHGTRLDIK |
| 349 | Anti- | RYTMH |
| | ITGav/CD51 | |
| | CDR-H1 | |
| 350 | Anti- | VISFDGSNKYYVDSVKG |
| | ITGav/CD51 | |
| | CDR-H2 | |
| 351 | Anti- | EARGSYAFDI |
| | ITGav/CD51 | |
| | CDR-H3 | |
| 352 | Anti- | RASQSVSSYLA |
| | ITGav/CD51 | |
| | CDR-L1 | |
| 353 | Anti- | DASNRAT |
| | ITGav/CD51 | |
| | CDR-L2 | |
| 354 | Anti- | QQRSNWPPFT |
| | ITGav/CD51 | |
| | CDR-L3 | |
| 355 | Anti- | QVQLVESGGGVVQPGRSRRLSCAASGFTFSRYTMHWVRQAPGKGLEWVAVISEDGSNKYY |
| | ITGav/CD51 | VDSVKGRFTISRDNSENTLYLQVNILRAEDTAVYYCAREARGSYAFDIWGQGTMVTVSS |
| | VH | |
| 356 | Anti- | EIVLTQSPATLSLSPGERATLSCRASQSVSSYLAWYQQKPGQAPRLLIYDASNRATGIPA |
| | ITGav/CD51 | RFSGSGSGTDFTLTISSLEPEDFAVYYCQQRSNWPPFTFGPGTKVDIK |
| | VL | |
| 357 | Anti-ITGav | SFWMH |
| | CDR-H1 | |
| 358 | Anti-ITGav | YINPRSGYTEYNEIFRD |
| | CDR-H2 | |
| 359 | Anti-ITGav | FLGRGAMDY |
| | CDR-H3 | |
| 360 | Anti-ITGav | RASQDISNYLA |
| | CDR-L1 | |
| 361 | Anti-ITGav | YTSKIHS |
| | CDR-L2 | |
| 362 | Anti-ITGav | QQGNTFPYT |
| | CDR-L3 | |
| 363 | Anti-ITGav | QVQLQQSGGELAKPGASVKVSCKASGYTFSSFWMHWVRQAPGQGLEWIGYINPRSGYTEY |
| | VH | NEIFRDKATMTTDTSTSTAYMELSSLRSEDTAVYYCASFLGRGAMDYWGQGTTVTVSS |
| 364 | Anti-ITGav | DIQMTQSPSSLSASVGDRVTITCRASQDISNYLAWYQQKPGKAPKLLIYYTSKIHSGVPS |
| | VL | RFSGSGSGTDYTFTISSLQPEDIATYYCQQGNTFPYTFGQGTKVEIK |
| 365 | Anti-gpA33 | TSSYYWG |
| | CDR-H1 | |
| 366 | Anti-gpA33 | TIYYNGSTYYSPSLKS |
| | CDR-H2 | |
| 367 | Anti-gpA33 | QGYDIKINIDV |
| | CDR-H3 | |
| 368 | Anti-gpA33 | RASQSVSSYLA |
| | CDR-L1 | |
| 369 | Anti-gpA33 | VASNRAT |
| | CDR-L2 | |
| 370 | Anti-gpA33 | QQRSNWPLT |
| | CDR-L3 | |
| 371 | Anti-gpA33 | QLQLQESGPGLVKPSETLSLTCTVSGGSISTSSYYWGWIRQPPGKGLEWIGTIYYNGSTY |
| | VH | YSPSLKSRVSISVDTSKNQFSLKLSSVTAADTSVYYCARQGYDIKINIDVWGQGTTVTVS |
| | | S |
| 372 | Anti-gpA33 | EIVLTQSPATLSLSPGERATLSCRASQSVSSYLAWYQQKPGQAPRLLIYVASNRATGIPA |
| | VL | RFSGSGSGTDFTLTISSLEPEDFAVYYCQQRSNWPLTFGGGTKVEIK |
| 373 | Anti-IL1Rap | SSWMN |
| | CDR-H1 | |
| 374 | Anti-IL1Rap | RIYPGDGNTHYAQKFQG |
| | CDR-H2 | |
| 375 | Anti-IL1Rap | GYLDPMDY |
| | CDR-H3 | |
| 376 | Anti-IL1Rap | QASQGINNYLN |
| | CDR-L1 | |
| 377 | Anti-IL1Rap | YTSGLHA |
| | CDR-L2 | |
| 378 | Anti-IL1Rap | QQYSILPWT |
| | CDR-L3 | |
| 379 | Anti-IL1Rap | QVQLVQSGAEVKKPGSSVKVSCKASGYAFTSSWMNWVRQAPGQGLEWMGRIYPGDGNTHY |
| | VH | AQKFQGRVTLTADKSTSTAYMELSSLRSEDTAVYYCGEGYLDPMDYWGQGTLVTVSS |
| 380 | Anti-IL1Rap | DIQMTQSPSSLSASVGDRVTITCQASQGINNYLNWYQQKPGKAPKLLIHYTSGLHAGVPS |
| | VL | RFSGSGSGTDYTLTISSLEPEDVATYYCQQYSILPWTFGGGTKVEIK |
| 381 | Anti-EpCAM | SYGMH |
| | CDR-H1 | |
| 382 | Anti-EpCAM | VISYDGSNKYYADSVKG |
| | CDR-H2 | |
| 383 | Anti-EpCAM | DMGWGSGWRPYYYYGMDV |
| | CDR-H3 | |
| 384 | Anti-EpCAM | RTSQSISSYLN |
| | CDR-L1 | |
| 385 | Anti-EpCAM | WASTRES |
| | CDR-L2 | |
| 386 | Anti-EpCAM | QQSYDIPYT |
| | CDR-L3 | |
| 387 | Anti-EpCAM | EVQLLESGGGVVQPGRSLRLSCAASGFTFSSYGMHWVRQAPGKGLEWVAVISYDGSNKYY |
| | VH | ADSVKGRFTISRDNSKNTLYLQMNSLRAEDTAVYYCAKDMGWGSGWRPYYYYGMDVWGQG |
| | | TTVTVSS |
| 388 | Anti-EpCAM | ELQMTQSPSSLSASVGDRVTITCRTSQSISSYLNWYQQKPGQPPKLLIYWASTRESGVPD |
| | VL | RFSGSGSGTDFTLTISSLQPEDSATYYCQQSYDIPYTFGQGTKLEIK |
| 389 | Anti-EpCAM | NYWMS |
| | CDR-H1 | |
| 390 | Anti-EpCAM | NIKQDGSEKFYADSVKG |
| | CDR-H2 | |
| 391 | Anti-EpCAM | VGPSWEQDY |
| | CDR-H3 | |
| 392 | Anti-EpCAM | TGSSSNIGSYYGVH |
| | CDR-L1 | |
| 393 | Anti-EpCAM | SDTNRPS |
| | CDR-L2 | |
| 394 | Anti-EpCAM | QSYDKGFGHRV |
| | CDR-L3 | |
| 395 | Anti-EpCAM | EVQLVESGGGLVQPGGSLRLSCAASGFTFSNYWMSWVRQAPGKGLEWVANIKQDGSEKFY |
| | VH | ADSVKGRFTISRDNAKNSLYLQMNSLRAEDTAVYYCARVGPSWEQDYWGQGTLVTVSA |
| 396 | Anti-EpCAM | QSVLTQPPSVSGAPGQRVTISCTGSSSNIGSYYGVHWYQQLPGTAPKLLIYSDTNRPSGV |
| | VL | PDRFSGSKSGTSASLAITGLQAEDEADYYCQSYDKGFGHRVFGGGTKLTVL |
| 397 | Anti-EpCAM | SYAIS |
| | CDR-H1 | |
| 398 | Anti-EpCAM | GIIPIFGTANYAQKFQG |
| | CDR-H2 | |
| 399 | Anti-EpCAM | GLLWNY |
| | CDR-H3 | |
| 400 | Anti-EpCAM | RASQSVSSNLA |
| | CDR-L1 | |
| 401 | Anti-EpCAM | GASTTAS |
| | CDR-L2 | |
| 402 | Anti-EpCAM | QQYNNWPPAYT |
| | CDR-L3 | |
| 403 | Anti-EpCAM | QVQLVQSGAEVKKPGSSVKVSCKASGGTFSSYAISWVRQAPGQGLEWMGGIIPIFGTANY |
| | VH | AQKFQGRVTITADESTSTAYMELSSLRSEDTAVYYCARGLLWNYWGQGTLVTVSS |
| 404 | Anti-EpCAM | EIVMTQSPATLSVSPGERATLSCRASQSVSSNLAWYQQKPGQAPRLIIYGASTTASGIPA |
| | VL | RFSASGSGTDFTLTISSLQSEDFAVYYCQQYNNWPPAYTFGQGTKLEIK |
| 405 | Anti-EpCAM | NYGMN |
| | CDR-H1 | |
| 406 | Anti-EpCAM | WINTYTGEPTYGEDFKG |
| | CDR-H2 | |
| 407 | Anti-EpCAM | FGNYVDY |
| | CDR-H3 | |
| 408 | Anti-EpCAM | RSSKNLLHSNGITYLY |
| | CDR-L1 | |
| 409 | Anti-EpCAM | QMSNLAS |
| | CDR-L2 | |
| 410 | Anti-EpCAM | AQNLEIPRT |
| | CDR-L3 | |
| 411 | Anti-EpCAM | QVQLVQSGPEVKKPGASVKVSCKASGYTFTNYGMNWVRQAPGQGLEWMGWINTYTGEPTY |
| | VH | GEDFKGRFAFSLDTSASTAYMELSSLRSEDTAVYFCARFGNYVDYWGQGSLVTVSS |
| 412 | Anti-EpCAM | DIVMTQSPLSLPVTPGEPASISCRSSKNLLHSNGITYLYWYLQKPGQSPQLLIYQMSNLA |
| | VL | SGVPDRFSSSGSGTDFTLKISRVEAEDVGVYYCAQNLEIPRTFGQGTKVEIK |
| 413 | Anti-EpCAM | KYGMN |
| | CDR-H1 | |
| 414 | Anti-EpCAM | WINTYTEEPTYGDDFKG |
| | CDR-H2 | |
| 415 | Anti-EpCAM | FGSAVDY |
| | CDR-H3 | |
| 416 | Anti-EpCAM | RSSKSLLHSNGITYLY |
| | CDR-L1 | |
| 417 | Anti-EpCAM | QMSNRAS |
| | CDR-L2 | |
| 418 | Anti-EpCAM | AQNLELPRT |
| | CDR-L3 | |
| 419 | Anti-EpCAM | QIQLVQSGPEVKKPGESVKISCKASGYTFTKYGMNWVKQAPGQGLKWMGWINTYTEEPTY |
| | VH | GDDFKGRFTFTLDTSTSTAYLEISSLRSEDTATYFCARFGSAVDYWGQGTLVTVSS |
| 420 | Anti-EpCAM | DIVMTQSALSNPVTLGESGSISCRSSKSLLHSNGITYLYWYLQKPGQSPQLLIYQMSNRA |
| | VL | SGVPDRESSSGSGTDFTLKISRVEAEDVGVYYCAQNLELPRTFGQGTKLEMKR |
| 421 | Anti-EpCAM | DYSMH |
| | CDR-H1 | |
| 422 | Anti-EpCAM | WINTETGEPTYADDFKG |
| | CDR-H2 | |
| 423 | Anti-EpCAM | TAVY |
| | CDR-H3 | |
| 424 | Anti-EpCAM | RASQEISVSLS |
| | CDR-L1 | |
| 425 | Anti-EpCAM | ATSTLDS |
| | CDR-L2 | |
| 426 | Anti-EpCAM | LQYASYPWT |
| | CDR-L3 | |
| 427 | Anti-EpCAM | QVKLQESGPELKKPGETVKISCKASGYTFTDYSMHWVKQAPGKGLKWMGWINTETGEPTY |
| | VH | ADDFKGRFAFSLETSASTAYLQINNLKNEDTATYFCARTAVYWGQGTTVTVSS |
| 428 | Anti-EpCAM | DIQMTQSPSSLSASLGERVSLTCRASQEISVSLSWLQQEPDGTIKRLIYATSTLDSGVPK |
| | VL | RFSGSRSGSDYSLTISSLESEDFVDYYCLQYASYPWTFGGGTKLEIKR |
| 429 | Anti-CD352 | NYGMN |
| | CDR-H1 | |
| 430 | Anti-CD352 | WINTYSGEPRYADDFKG |
| | CDR-H2 | |
| 431 | Anti-CD352 | DYGRWYFDV |
| | CDR-H3 | |
| 432 | Anti-CD352 | RASSSVSHMH |
| | CDR-L1 | |
| 433 | Anti-CD352 | ATSNLAS |
| | CDR-L2 | |
| 434 | Anti-CD352 | QQWSSTPRT |
| | CDR-L3 | |
| 435 | Anti-CD352 | QIQLVQSGSELKKPGASVKVSCKASGYTFTNYGMNWVRQAPGQDLKWMGWINTYSGEPRY |
| | VH | ADDFKGRFVFSLDKSVNTAYLQISSLKAEDTAVYYCARDYGRWYFDVWGQGTTVTVSS |
| 436 | Anti-CD352 | QIVLSQSPATLSLSPGERATMSCRASSSVSHMHWYQQKPGQAPRPWIYATSNLASGVPAR |
| | VL | FSGSGSGTDYTLTISSLEPEDFAVYYCQQWSSTPRTFGGGTKVEIKR |
| 437 | Anti-CS1 | RYWMS |
| | CDR-H1 | |
| 438 | Anti-CS1 | EINPDSSTINYAPSLKD |
| | CDR-H2 | |
| 439 | Anti-CS1 | PDGNYWYFDV |
| | CDR-H3 | |
| 440 | Anti-CS1 | KASQDVGIAVA |
| | CDR-L1 | |
| 441 | Anti-CS1 | WASTRHT |
| | CDR-L2 | |
| 442 | Anti-CS1 | QQYSSYPYT |
| | CDR-L3 | |
| 443 | Anti-CS1 VH | EVQLVESGGGLVQPGGSLRLSCAASGFDFSRYWMSWVRQAPGKGLEWIGEINPDSSTINY |
| | | APSLKDKFIISRDNAKNSLYLQMNSLRAEDTAVYYCARPDGNYWYFDVWGQGTLVTVSS |
| 444 | Anti-CS1 VL | DIQMTQSPSSLSASVGDRVTITCKASQDVGIAVAWYQQKPGKVPKLLIYWASTRHTGVPD |
| | | RFSGSGSGTDFTLTISSLQPEDVATYYCQQYSSYPYTFGQGTKVEIKR |
| 445 | Anti-CD38 | SFAMS |
| | CDR-H1 | |
| 446 | Anti-CD38 | AISGSGGGTYYADSVKG |
| | CDR-H2 | |
| 447 | Anti-CD38 | DKILWFGEPVFDY |
| | CDR-H3 | |
| 448 | Anti-CD38 | RASQSVSSYLA |
| | CDR-L1 | |
| 449 | Anti-CD38 | DASNRAT |
| | CDR-L2 | |
| 450 | Anti-CD38 | QQRSNWPPT |
| | CDR-L3 | |
| 451 | Anti-CD38 | EVQLLESGGGLVQPGGSLRLSCAVSGFTFNSFAMSWVRQAPGKGLEWVSAISGSGGGTYY |
| | VH | ADSVKGRFTISRDNSKNTLYLQMNSLRAEDTAVYFCAKDKILWFGEPVFDYWGQGTLVTV |
| | | SS |
| 452 | Anti-CD38 | EIVLTQSPATLSLSPGERATLSCRASQSVSSYLAWYQQKPGQAPRLLIYDASNRATGIPA |
| | VL | RFSGSGSGTDFTLTISSLEPEDFAVYYCQQRSNWPPTFGQGTKVEIKR |
| 453 | Anti-CD25 | SYRMH |
| | CDR-H1 | |
| 454 | Anti-CD25 | YINPSTGYTEYNQKFKD |
| | CDR-H2 | |
| 455 | Anti-CD25 | GGGVFDY |
| | CDR-H3 | |
| 456 | Anti-CD25 | SASSSISYMH |
| | CDR-L1 | |
| 457 | Anti-CD25 | TTSNLAS |
| | CDR-L2 | |
| 458 | Anti-CD25 | HQRSTYPLT |
| | CDR-L3 | |
| 459 | Anti-CD25 | QVQLVQSGAEVKKPGSSVKVSCKASGYTFTSYRMHWVRQAPGQGLEWIGYINPSTGYTEY |
| | VH | NQKFKDKATITADESTNTAYMELSSLRSEDTAVYYCARGGGVFDYWGQGTLVTVSS |
| 460 | Anti-CD25 | DIQMTQSPSTLSASVGDRVTITCSASSSISYMHWYQQKPGKAPKLLIYTTSNLASGVPAR |
| | VL | FSGSGSGTEFTLTISSLQPDDFATYYCHQRSTYPLTFGQGTKVEVK |
| 461 | Anti-ADAM9 | SYWMH |
| | CDR-H1 | |
| 462 | Anti-ADAM9 | EIIPINGHTNYNEKFKS |
| | CDR-H2 | |
| 463 | Anti-ADAM9 | GGYYYYGSRDYFDY |
| | CDR-H3 | |
| 464 | Anti-ADAM9 | KASQSVDYDGDSYMN |
| | CDR-L1 | |
| 465 | Anti-ADAM9 | AASDLES |
| | CDR-L2 | |
| 466 | Anti-ADAM9 | QQSHEDPFT |
| | CDR-L3 | |
| 467 | Anti-ADAM9 | QVQLQQPGAELVKPGASVKLSCKASGYTFTSYWMHWVKQRPGQGLEWIGEIIPINGHTNY |
| | VH | NEKFKSKATLTLDKSSSTAYMQLSSLASEDSAVYYCARGGYYYYGSRDYFDYWGQGTTLT |
| | | VSS |
| 468 | Anti-ADAM9 | DIVLTQSPASLAVSLGQRATISCKASQSVDYDGDSYMNWYQQIPGQPPKLLIYAASDLES |
| | VL | GIPARFSGSGSGTDFTLNIHPVEEEDAATYYCQQSHEDPFTFGGGTKLEIK |
| 469 | Anti-ADAM9 | SYWMH |
| | CDR-H1 | |
| 470 | Anti-ADAM9 | EIIPIFGHTNYNEKFKS |
| | CDR-H2 | |
| 471 | Anti-ADAM9 | GGYYYYPRQGFLDY |
| | CDR-H3 | |
| 472 | Anti-ADAM9 | KASQSVDYSGDSYMN |
| | CDR-L1 | |
| 473 | Anti-ADAM9 | AASDLES |
| | CDR-L2 | |
| 474 | Anti-ADAM9 | QQSHEDPFT |
| | CDR-L3 | |
| 475 | Anti-ADAM9 | EVQLVESGGGLVKPGGSLRLSCAASGFTFSSYWMHWVRQAPGKGLEWVGEIIPIFGHTNY |
| | VH | NEKFKSRFTISLDNSKNTLYLQMGSLRAEDTAVYYCARGGYYYYPRQGFLDYWGQGTTVT |
| | | VSS |
| 476 | Anti-ADAM9 | DIVMTQSPDSLAVSLGERATISCKASQSVDYSGDSYMNWYQQKPGQPPKLLIYAASDLES |
| | VL | GIPARFSGSGSGTDFTLTISSLEPEDFATYYCQQSHEDPFTFGQGTKLEIK |
| 477 | Anti-CD59 | SYGMN |
| | CDR-H1 | |
| 478 | Anti-CD59 | YISSSSSTIYYADSVKG |
| | CDR-H2 | |
| 479 | Anti-CD59 | GPGMDV |
| | CDR-H3 | |
| 480 | Anti-CD59 | KSSQSVLYSSNNKNYLA |
| | CDR-L1 | |
| 481 | Anti-CD59 | WASTRES |
| | CDR-L2 | |
| 482 | Anti-CD59 | QQYYSTPQLT |
| | CDR-L3 | |
| 483 | Anti-CD59 | QVQLQQSGGGVVQPGRSLGLSCAASGFTFSSYGMNWVRQAPGKGLEWVSYISSSSSTIYY |
| | VH | ADSVKGRFTISRDNSKNTLYLQMNSLRAEDTAVYYCARGPGMDVWGQGTTVTVS |
| 484 | Anti-CD59 | DIVLTQSPDSLAVSLGERATINCKSSQSVLYSSNNKNYLAWYQQKPGQPPKLLIYWASTR |
| | VL | ESGVPDRFSGSGSGTDFTPAISSLQAEDVAVYYCQQYYSTPQLTFGGGTKVDIK |
| 485 | Anti-CD19 | TSGMGVG |
| | (hBU12) | |
| | CDR-H1 | |
| 486 | Anti-CD19 | HIWWDDDKRYNPALKS |
| | (hBU12) | |
| | CDR-H2 | |
| 487 | Anti-CD19 | MELWSYYFDY |
| | (hBU12) | |
| | CDR-H3 | |
| 488 | Anti-CD19 | SASSSVSYMH |
| | (hBU12) | |
| | CDR-L1 | |
| 489 | Anti-CD19 | DTSKLAS |
| | (hBU12) | |
| | CDR-L2 | |
| 490 | Anti-CD19 | FQGSVYPFT |
| | (hBU12) | |
| | CDR-L3 | |
| 491 | Anti-CD19 | QVQLQESGPGLVKPSQTLSLTCTVSGGSISTSGMGVGWIRQHPGKGLEWIGHIWWDDDKR |
| | (hBU12) VH | YNPALKSRVTISVDTSKNQFSLKLSSVTAADTAVYYCARMELWSYYFDYWGQGTLVTVSS |
| 492 | Anti-CD19 | EIVLTQSPATLSLSPGERATLSCSASSSVSYMHWYQQKPGQAPRLLIYDTSKLASGIPAR |
| | (hBU12) VL | FSGSGSGTDFTLTISSLEPEDVAVYYCFQGSVYPFTFGQGTKLEIKR |
| 493 | Anti-CD19 | QVQLQESGPGLVKPSQTLSLTCTVSGGSISTSGMGVGWIRQHPGKGLEWIGHIWWDDDKR |
| | (hBU12) HC | YNPALKSRVTISVDTSKNQFSLKLSSVTAADTAVYYCARMELWSYYFDYWGQGTLVTVSS |
| | | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 494 | Anti-CD19 | EIVLTQSPATLSLSPGERATLSCSASSSVSYMHWYQQKPGQAPRLLIYDTSKLASGIPAR |
| | (hBU12) LC | FSGSGSGTDFTLTISSLEPEDVAVYYCFQGSVYPFTFGQGTKLEIKRTVAAPSVFIFPPS |
| | | DEQLKSGTASVVCLLNNFYPREAKVQWKVDNALQSGNSQESVTEQDSKDSTYSLSSTLTL |
| | | SKADYEKHKVYACEVTHQGLSSPVTKSENRGEC |
| 495 | Anti-CD138 | NYWIE |
| | CDR-H1 | |
| 496 | Anti-CD138 | EILPGTGRTIYNEKFKG |
| | CDR-H2 | |
| 497 | Anti-CD138 | RDYYGNFYYAMDY |
| | CDR-H3 | |
| 498 | Anti-CD138 | SASQGINNYLN |
| | CDR-L1 | |
| 499 | Anti-CD138 | YTSTLQS |
| | CDR-L2 | |
| 500 | Anti-CD138 | QQYSKLPRT |
| | CDR-L3 | |
| 501 | Anti-CD138 | QVQLQQSGSELMMPGASVKISCKATGYTFSNYWIEWVKQRPGHGLEWIGEILPGTGRTIY |
| | VH | NEKFKGKATFTADISSNTVQMQLSSLTSEDSAVYYCARRDYYGNFYYAMDYWGQGTSVTV |
| | | SS |
| 502 | Anti-CD138 | DIQMTQSTSSLSASLGDRVTISCSASQGINNYLNWYQQKPDGTVELLIYYTSTLQSGVPS |
| | VL | RFSGSGSGTDYSLTISNLEPEDIGTYYCQQYSKLPRTFGGGTKLEIK |
| 503 | Anti-CD166 | TYGMGVG |
| | CDR-H1 | |
| 504 | Anti-CD166 | NIWWSEDKHYSPSLKS |
| | CDR-H2 | |
| 505 | Anti-CD166 | IDYGNDYAFTY |
| | CDR-H3 | |
| 506 | Anti-CD166 | RSSKSLLHSNGITYLY |
| | CDR-L1 | |
| 507 | Anti-CD166 | QMSNLAS |
| | CDR-L2 | |
| 508 | Anti-CD166 | AQNLELPYT |
| | CDR-L3 | |
| 509 | Anti-CD166 | QITLKESGPTLVKPTQTLTLTCTFSGFSLSTYGMGVGWIRQPPGKALEWLANIWWSEDKH |
| | VH | YSPSLKSRLTITKDTSKNQVVLTITNVDPVDTATYYCVQIDYGNDYAFTYWGQGTLVTVS |
| | | S |
| 510 | Anti-CD166 | DIVMTQSPLSLPVTPGEPASISCRSSKSLLHSNGITYLYWYLQKPGQSPQLLIYQMSNLA |
| | VL | SGVPDRFSGSGSGTDFTLKISRVEAEDVGVYYCAQNLELPYTFGQGTKLEIK |
| 511 | Anti-CD56 | SFGMH |
| | CDR-H1 | |
| 512 | Anti-CD56 | YISSGSFTIYYADSVKG |
| | CDR-H2 | |
| 513 | Anti-CD56 | MRKGYAMDY |
| | CDR-H3 | |
| 514 | Anti-CD56 | RSSQIIIHSDGNTYLE |
| | CDR-L1 | |
| 515 | Anti-CD56 | KVSNRFS |
| | CDR-L2 | |
| 516 | Anti-CD56 | FQGSHVPHT |
| | CDR-L3 | |
| 517 | Anti-CD56 | QVQLVESGGGVVQPGRSLRLSCAASGFTFSSFGMHWVRQAPGKGLEWVAYISSGSFTIYY |
| | VH | ADSVKGRFTISRDNSKNTLYLQMNSLRAEDTAVYYCARMRKGYAMDYWGQGTLVTVSS |
| 518 | Anti-CD56 | DVVMTQSPLSLPVTLGQPASISCRSSQIIIHSDGNTYLEWFQQRPGQSPRRLIYKVSNRF |
| | VL | SGVPDRFSGSGSGTDFTLKISRVEAEDVGVYYCFQGSHVPHTFGQGTKVEIK |
| 519 | Anti-CD74 | NYGVN |
| | CDR-H1 | |
| 520 | Anti-CD74 | WINPNTGEPTFDDDFKG |
| | CDR-H2 | |
| 521 | Anti-CD74 | SRGKNEAWFAY |
| | CDR-H3 | |
| 522 | Anti-CD74 | RSSQSLVHRNGNTYLH |
| | CDR-L1 | |
| 523 | Anti-CD74 | TVSNRFS |
| | CDR-L2 | |
| 524 | Anti-CD74 | SQSSHVPPT |
| | CDR-L3 | |
| 525 | Anti-CD74 | QVQLQQSGSELKKPGASVKVSCKASGYTFTNYGVNWIKQAPGQGLQWMGWINPNTGEPTF |
| | VH | DDDFKGRFAFSLDTSVSTAYLQISSLKADDTAVYFCSRSRGKNEAWFAYWGQGTLVTVSS |
| 526 | Anti-CD74 | DIQLTQSPLSLPVTLGQPASISCRSSQSLVHRNGNTYLHWFQQRPGQSPRLLIYTVSNRF |
| | VL | SGVPDRFSGSGSGTDFTLKISRVEAEDVGVYFCSQSSHVPPTFGAGTRLEIK |
| 527 | Anti- | TYWMS |
| | CEACAM5 | |
| | CDR-H1 | |
| 528 | Anti- | EIHPDSSTINYAPSLKD |
| | CEACAM5 | |
| | CDR-H2 | |
| 529 | Anti- | LYFGFPWFAY |
| | CEACAM5 | |
| | CDR-H3 | |
| 530 | Anti- | KASQDVGTSVA |
| | CEACAM5 | |
| | CDR-L1 | |
| 531 | Anti- | WTSTRHT |
| | CEACAM5 | |
| | CDR-L2 | |
| 532 | Anti- | QQYSLYRS |
| | CEACAM5 | |
| | CDR-L3 | |
| 533 | Anti- | EVQLVESGGGVVQPGRSLRLSCSASGFDFTTYWMSWVRQAPGKGLEWIGEIHPDSSTINY |
| | CEACAM5 VH | APSLKDRFTISRDNAKNTLFLQMDSLRPEDTGVYFCASLYFGFPWFAYWGQGTPVTVSS |
| 534 | Anti- | DIQLTQSPSSLSASVGDRVTITCKASQDVGTSVAWYQQKPGKAPKLLIYWTSTRHTGVPS |
| | CEACAM5 VL | RFSGSGSGTDFTFTISSLQPEDIATYYCQQYSLYRSFGQGTKVEIK |
| 535 | Anti-CanAg | YYGMN |
| | CDR-H1 | |
| 536 | Anti-CanAg | WIDTTTGEPTYAQKFQG |
| | CDR-H2 | |
| 537 | Anti-CanAg | RGPYNWYFDV |
| | CDR-H3 | |
| 538 | Anti-CanAg | RSSKSLLHSNGNTYLY |
| | CDR-L1 | |
| 539 | Anti-CanAg | RMSNLVS |
| | CDR-L2 | |
| 540 | Anti-CanAg | LQHLEYPFT |
| | CDR-L3 | |
| 541 | Anti-CanAg | QVQLVQSGAEVKKPGETVKISCKASDYTFTYYGMNWVKQAPGQGLKWMGWIDTTTGEPTY |
| | VH | AQKFQGRIAFSLETSASTAYLQIKSLKSEDTATYFCARRGPYNWYFDVWGQGTTVTVSS |
| 542 | Anti-CanAg | DIVMTQSPLSVPVTPGEPVSISCRSSKSLLHSNGNTYLYWFLQRPGQSPQLLIYRMSNLV |
| | VL | SGVPDRFSGSGSGTAFTLRISRVEAEDVGVYYCLQHLEYPFTFGPGTKLELK |
| 543 | Anti-DLL-3 | NYGMN |
| | CDR-H1 | |
| 544 | Anti-DLL-3 | WINTYTGEPTYADDFKG |
| | CDR-H2 | |
| 545 | Anti-DLL-3 | IGDSSPSDY |
| | CDR-H3 | |
| 546 | Anti-DLL-3 | KASQSVSNDVV |
| | CDR-L1 | |
| 547 | Anti-DLL-3 | YASNRYT |
| | CDR-L2 | |
| 548 | Anti-DLL-3 | QQDYTSPWT |
| | CDR-L3 | |
| 549 | Anti-DLL-3 | QVQLVQSGAEVKKPGASVKVSCKASGYTFTNYGMNWVRQAPGQGLEWMGWINTYTGEPTY |
| | VH | ADDFKGRVTMTTDTSTSTAYMELRSLRSDDTAVYYCARIGDSSPSDYWGQGTLVTVSS |
| 550 | Anti-DLL-3 | EIVMTQSPATLSVSPGERATLSCKASQSVSNDVVWYQQKPGQAPRLLIYYASNRYTGIPA |
| | VL | RFSGSGSGTEFTLTISSLQSEDFAVYYCQQDYTSPWTFGQGTKLEIK |
| 551 | Anti-DPEP-3 | SYWIE |
| | CDR-H1 | |
| 552 | Anti-DPEP-3 | EILPGSGNTYYNERFKD |
| | CDR-H2 | |
| 553 | Anti-DPEP-3 | RAAAYYSNPEWFAY |
| | CDR-H3 | |
| 554 | Anti-DPEP-3 | TASSSVNSFYLH |
| | CDR-L1 | |
| 555 | Anti-DPEP-3 | STSNLAS |
| | CDR-L2 | |
| 556 | Anti-DPEP-3 | HQYHRSPYT |
| | CDR-L3 | |
| 557 | Anti-DPEP-3 | QVQLVQSGAEVKKPGSSVKVSCKASGGTFSSYWIEWVRQAPGQGLEWMGEILPGSGNTYY |
| | VH | NERFKDRVTITADESTSTAYMELSSLRSEDTAVYYCARRAAAYYSNPEWFAYWGQGTLVT |
| | | VSS |
| 558 | Anti-DPEP-3 | EIVLTQSPATLSLSPGERATLSCTASSSVNSFYLHWYQQKPGLAPRLLIYSTSNLASGIP |
| | VL | DRFSGSGSGTDFTLTISRLEPEDFAVYYCHQYHRSPYTFGQGTKLEIK |
| 559 | Anti-EGFR | SYWMQ |
| | CDR-H1 | |
| 560 | Anti-EGFR | TIYPGDGDTTYTQKFQG |
| | CDR-H2 | |
| 561 | Anti-EGFR | YDAPGYAMDY |
| | CDR-H3 | |
| 562 | Anti-EGFR | RASQDINNYLA |
| | CDR-L1 | |
| 563 | Anti-EGFR | YTSTLHP |
| | CDR-L2 | |
| 564 | Anti-EGFR | LQYDNLLYT |
| | CDR-L3 | |
| 565 | Anti-EGFR | QVQLVQSGAEVAKPGASVKLSCKASGYTFTSYWMQWVKQRPGQGLECIGTIYPGDGDTTY |
| | VH | TQKFQGKATLTADKSSSTAYMQLSSLRSEDSAVYYCARYDAPGYAMDYWGQGTLVTVSS |
| 566 | Anti-EGFR | DIQMTQSPSSLSASVGDRVTITCRASQDINNYLAWYQHKPGKGPKLLIHYTSTLHPGIPS |
| | VL | RFSGSGSGRDYSFSISSLEPEDIATYYCLQYDNLLYTFGQGTKLEIK |
| 567 | Anti-EGFR | RDFAWN |
| | CDR-H1 | |
| 568 | Anti-EGFR | YISYNGNTRYQPSLKS |
| | CDR-H2 | |
| 569 | Anti-EGFR | ASRGFPY |
| | CDR-H3 | |
| 570 | Anti-EGFR | HSSQDINSNIG |
| | CDR-L1 | |
| 571 | Anti-EGFR | HGTNLDD |
| | CDR-L2 | |
| 572 | Anti-EGFR | VQYAQFPWT |
| | CDR-L3 | |
| 573 | Anti-EGFR | EVQLQESGPGLVKPSQTLSLTCTVSGYSISRDFAWNWIRQPPGKGLEWMGYISYNGNTRY |
| | VH | QPSLKSRITISRDTSKNQFFLKLNSVTAADTATYYCVTASRGFPYWGQGTLVTVSS |
| 574 | Anti-EGFR | DIQMTQSPSSMSVSVGDRVTITCHSSQDINSNIGWLQQKPGKSFKGLIYHGTNLDDGVPS |
| | VL | RFSGSGSGTDYTLTISSLQPEDFATYYCVQYAQFPWTFGGGTKLEIK |
| 575 | Anti-EGFR | NYGVH |
| | CDR-H1 | |
| 576 | Anti-EGFR | VIWSGGNTDYNTPFTS |
| | CDR-H2 | |
| 577 | Anti-EGFR | ALTYYDYEFAY |
| | CDR-H3 | |
| 578 | Anti-EGFR | RASQSIGTNIH |
| | CDR-L1 | |
| 579 | Anti-EGFR | YASESIS |
| | CDR-L2 | |
| 580 | Anti-EGFR | QQNNNWPTT |
| | CDR-L3 | |
| 581 | Anti-EGFR | QVQLKQSGPGLVQPSQSLSITCTVSGFSLTNYGVHWVRQSPGKGLEWLGVIWSGGNTDYN |
| | VH | TPFTSRLSINKDNSKSQVFFKMNSLQSNDTAIYYCARALTYYDYEFAYWGQGTLVTVSA |
| 582 | Anti-EGFR | DILLTQSPVILSVSPGERVSFSCRASQSIGTNIHWYQQRINGSPRLLIKYASESISGIPS |
| | VL | RFSGSGSGTDFTLSINSVESEDIADYYCQQNNNWPTTFGAGTKLELK |
| 583 | Anti-FRa | GYFMN |
| | CDR-H1 | |
| 584 | Anti-FRa | RIHPYDGDTFYNQKFQG |
| | CDR-H2 | |
| 585 | Anti-FRa | YDGSRAMDY |
| | CDR-H3 | |
| 586 | Anti-FRa | KASQSVSFAGTSLMH |
| | CDR-L1 | |
| 587 | Anti-FRa | RASNLEA |
| | CDR-L2 | |
| 588 | Anti-FRa | QQSREYPYT |
| | CDR-L3 | |
| 589 | Anti-FRa VH | QVQLVQSGAEVVKPGASVKISCKASGYTFTGYFMNWVKQSPGQSLEWIGRIHPYDGDTFY |
| | | NQKFQGKATLTVDKSSNTAHMELLSLTSEDFAVYYCTRYDGSRAMDYWGQGTTVTVSS |
| 590 | Anti-FRa VL | DIVLTQSPLSLAVSLGQPAIISCKASQSVSFAGTSLMHWYHQKPGQQPRLLIYRASNLEA |
| | | GVPDRFSGSGSKTDFTLTISPVEAEDAATYYCQQSREYPYTFGGGTKLEIK |
| 591 | Anti-FRa | GYGLS |
| | CDR-H1 | |
| 592 | Anti-FRa | MISSGGSYTYYADSVKG |
| | CDR-H2 | |
| 593 | Anti-FRa | HGDDPAWFAY |
| | CDR-H3 | |
| 594 | Anti-FRa | SVSSSISSNNLH |
| | CDR-L1 | |
| 595 | Anti-FRa | GTSNLAS |
| | CDR-L2 | |
| 596 | Anti-FRa | QQWSSYPYMYT |
| | CDR-L3 | |
| 597 | Anti-FRa VH | EVQLVESGGGVVQPGRSLRLSCSASGFTFSGYGLSWVRQAPGKGLEWVAMISSGGSYTYY |
| | | ADSVKGRFAISRDNAKNTLFLQMDSLRPEDTGVYFCARHGDDPAWFAYWGQGTPVTVSS |
| 598 | Anti-FRa VL | DIQLTQSPSSLSASVGDRVTITCSVSSSISSNNLHWYQQKPGKAPKPWIYGTSNLASGVP |
| | | SRFSGSGSGTDYTFTISSLQPEDIATYYCQQWSSYPYMYTFGQGTKVEIK |
| 599 | Anti-MUC-1 | NYWMN |
| | CDR-H1 | |
| 600 | Anti-MUC-1 | EIRLKSNNYTTHYAESVKG |
| | CDR-H2 | |
| 601 | Anti-MUC-1 | HYYFDY |
| | CDR-H3 | |
| 602 | Anti-MUC-1 | RSSKSLLHSNGITYFF |
| | CDR-L1 | |
| 603 | Anti-MUC-1 | QMSNLAS |
| | CDR-L2 | |
| 604 | Anti-MUC-1 | AQNLELPPT |
| | CDR-L3 | |
| 605 | Anti-MUC-1 | EVQLVESGGGLVQPGGSMRLSCVASGFPFSNYWMNWVRQAPGKGLEWVGEIRLKSNNYTT |
| | VH | HYAESVKGRFTISRDDSKNSLYLQMNSLKTEDTAVYYCTRHYYFDYWGQGTLVTVSS |
| 606 | Anti-MUC-1 | DIVMTQSPLSNPVTPGEPASISCRSSKSLLHSNGITYFFWYLQKPGQSPQLLIYQMSNLA |
| | VL | SGVPDRFSGSGSGTDFTLRISRVEAEDVGVYYCAQNLELPPTFGQGTKVEIK |
| 607 | Anti- | SYWIG |
| | Mesothelin | |
| | CDR-H1 | |
| 608 | Anti- | IIDPGDSRTRYSPSFQG |
| | Mesothelin | |
| | CDR-H2 | |
| 609 | Anti- | GQLYGGTYMDG |
| | Mesothelin | |
| | CDR-H3 | |
| 610 | Anti- | TGTSSDIGGYNSVS |
| | Mesothelin | |
| | CDR-L1 | |
| 611 | Anti- | GVNNRPS |
| | Mesothelin | |
| | CDR-L2 | |
| 612 | Anti- | SSYDIESATPV |
| | Mesothelin | |
| | CDR-L3 | |
| 613 | Anti- | QVELVQSGAEVKKPGESLKISCKGSGYSFTSYWIGWVRQAPGKGLEWMGIIDPGDSRTRY |
| | Mesothelin | SPSFQGQVTISADKSISTAYLQWSSLKASDTAMYYCARGQLYGGTYMDGWGQGTLVTVSS |
| | VH | |
| 614 | Anti- | DIALTQPASVSGSPGQSITISCTGTSSDIGGYNSVSWYQQHPGKAPKLMIYGVNNRPSGV |
| | Mesothelin | SNRFSGSKSGNTASLTISGLQAEDEADYYCSSYDIESATPVFGGGTKLTVL |
| | VL | |
| 615 | Anti-ROR-1 | AYNIH |
| | CDR-H1 | |
| 616 | Anti-ROR-1 | SFDPYDGGSSYNQKFKD |
| | CDR-H2 | |
| 617 | Anti-ROR-1 | GWYYFDY |
| | CDR-H3 | |
| 618 | Anti-ROR-1 | RASKSISKYLA |
| | CDR-L1 | |
| 619 | Anti-ROR-1 | SGSTLQS |
| | CDR-L2 | |
| 620 | Anti-ROR-1 | QQHDESPYT |
| | CDR-L3 | |
| 621 | Anti-ROR-1 | QVQLQESGPGLVKPSQTLSLTCTVSGYAFTAYNIHWVRQAPGQGLEWMGSFDPYDGGSSY |
| | VH | NQKFKDRLTISKDTSKNQVVLTMTNMDPVDTATYYCARGWYYFDYWGHGTLVTVSS |
| 622 | Anti-ROR-1 | DIVMTQTPLSLPVTPGEPASISCRASKSISKYLAWYQQKPGQAPRLLIYSGSTLQSGIPP |
| | VL | RFSGSGYGTDFTLTINNIESEDAAYYFCQQHDESPYTFGEGTKVEIK |
| 623 | Anti-B7-H3 | SFGMH |
| | CDR-H1 | |
| 624 | Anti-B7-H3 | YISSDSSAIYYADTVKG |
| | CDR-H2 | |
| 625 | Anti-B7-H3 | GRENIYYGSRLDY |
| | CDR-H3 | |
| 626 | Anti-B7-H3 | KASQNVDTNVA |
| | CDR-L1 | |
| 627 | Anti-B7-H3 | SASYRYS |
| | CDR-L2 | |
| 628 | Anti-B7-H3 | QQYNNYPFT |
| | CDR-L3 | |
| 629 | Anti-B7-H3 | DVQLVESGGGLVQPGGSRKLSCAASGFTFSSFGMHWVRQAPEKGLEWVAYISSDSSAIYY |
| | VH | ADTVKGRFTISRDNPKNTLFLQMTSLRSEDTAMYYCGRGRENIYYGSRLDYWGQGTTLTV |
| | | SS |
| 630 | Anti-B7-H3 | DIAMTQSQKFMSTSVGDRVSVTCKASQNVDTNVAWYQQKPGQSPKALIYSASYRYSGVPD |
| | VL | RFTGSGSGTDFTLTINNVQSEDLAEYFCQQYNNYPFTFGSGTKLEIK |
| 631 | Anti-B7-H3 | SYGMS |
| | CDR-H1 | |
| 632 | Anti-B7-H3 | TINSGGSNTYYPDSLKG |
| | CDR-H2 | |
| 633 | Anti-B7-H3 | HDGGAMDY |
| | CDR-H3 | |
| 634 | Anti-B7-H3 | RASESIYSYLA |
| | CDR-L1 | |
| 635 | Anti-B7-H3 | NTKTLPE |
| | CDR-L2 | |
| 636 | Anti-B7-H3 | QHHYGTPPWT |
| | CDR-L3 | |
| 637 | Anti-B7-H3 | EVQLVESGGGLVKPGGSLRLSCAASGFTFSSYGMSWVRQAPGKGLEWVATINSGGSNTYY |
| | VH | PDSLKGRFTISRDNAKNSLYLQMNSLRAEDTAVYYCARHDGGAMDYWGQGTTVTVSS |
| 638 | Anti-B7-H3 | DIQMTQSPSSLSASVGDRVTITCRASESIYSYLAWYQQKPGKAPKLLVYNTKTLPEGVPS |
| | VL | RFSGSGSGTDFTLTISSLQPEDFATYYCQHHYGTPPWTFGQGTRLEIK |
| 639 | Anti-B7-H3 | SFGMH |
| | CDR-H1 | |
| 640 | Anti-B7-H3 | YISSGSGTIYYADTVKG |
| | CDR-H2 | |
| 641 | Anti-B7-H3 | HGYRYEGFDY |
| | CDR-H3 | |
| 642 | Anti-B7-H3 | KASQNVDTNVA |
| | CDR-L1 | |
| 643 | Anti-B7-H3 | SASYRYS |
| | CDR-L2 | |
| 644 | Anti-B7-H3 | QQYNNYPFT |
| | CDR-L3 | |
| 645 | Anti-B7-H3 | EVQLVESGGGLVQPGGSLRLSCAASGFTFSSFGMHWVRQAPGKGLEWVAYISSGSGTIYY |
| | VH | ADTVKGRFTISRDNAKNSLYLQMNSLRAEDTAVYYCARHGYRYEGFDYWGQGTTVTVSS |
| 646 | Anti-B7-H3 | DIQMTQSPSFLSASVGDRVTITCKASQNVDTNVAWYQQKPGKAPKALIYSASYRYSGVPS |
| | VL | RFSGSGSGTDFTLTISSLQPEDFAEYFCQQYNNYPFTFGQGTKLEIK |
| 647 | Anti-B7-H3 | NYVMH |
| | CDR-H1 | |
| 648 | Anti-B7-H3 | YINPYNDDVKYNEKFKG |
| | CDR-H2 | |
| 649 | Anti-B7-H3 | WGYYGSPLYYFDY |
| | CDR-H3 | |
| 650 | Anti-B7-H3 | RASSRLIYMH |
| | CDR-L1 | |
| 651 | Anti-B7-H3 | ATSNLAS |
| | CDR-L2 | |
| 652 | Anti-B7-H3 | QQWNSNPPT |
| | CDR-L3 | |
| 653 | Anti-B7-H3 | EVQLQQSGPELVKPGASVKMSCKASGYTFTNYVMHWVKQKPGQGLEWIGYINPYNDDVKY |
| | VH | NEKFKGKATQTSDKSSSTAYMELSSLTSEDSAVYYCARWGYYGSPLYYFDYWGQGTTLTV |
| | | SS |
| 654 | Anti-B7-H3 | QIVLSQSPTILSASPGEKVTMTCRASSRLIYMHWYQQKPGSSPKPWIYATSNLASGVPAR |
| | VL | FSGSGSGTSYSLTISRVEAEDAATYYCQQWNSNPPTFGTGTKLELK |
| 655 | Anti-B7-H3 | NYVMH |
| | CDR-H1 | |
| 656 | Anti-B7-H3 | YINPYNDDVKYNEKFKG |
| | CDR-H2 | |
| 657 | Anti-B7-H3 | WGYYGSPLYYFDY |
| | CDR-H3 | |
| 658 | Anti-B7-H3 | RASSRLIYMH |
| | CDR-L1 | |
| 659 | Anti-B7-H3 | ATSNLAS |
| | CDR-L2 | |
| 660 | Anti-B7-H3 | QQWNSNPPT |
| | CDR-L3 | |
| 661 | Anti-B7-H3 | QVQLVQSGAEVKKPGSSVKVSCKASGYTFTNYVMHWVRQAPGQGLEWMGYINPYNDDVKY |
| | VH | NEKFKGRVTITADESTSTAYMELSSLRSEDTAVYYCARWGYYGSPLYYFDYWGQGTLVTV |
| | | SS |
| 662 | Anti-B7-H3 | EIVLTQSPATLSLSPGERATLSCRASSRLIYMHWYQQKPGQAPRPLIYATSNLASGIPAR |
| | VL | FSGSGSGTDFTLTISSLEPEDFAVYYCQQWNSNPPTFGQGTKVEIK |
| 663 | Anti-B7-H3 | SYTIH |
| | CDR-H1 | |
| 664 | Anti-B7-H3 | YINPNSRNTDYAQKFQG |
| | CDR-H2 | |
| 665 | Anti-B7-H3 | YSGSTPYWYFDV |
| | CDR-H3 | |
| 666 | Anti-B7-H3 | RASSSVSYMN |
| | CDR-L1 | |
| 667 | Anti-B7-H3 | ATSNLAS |
| | CDR-L2 | |
| 668 | Anti-B7-H3 | QQWSSNPLT |
| | CDR-L3 | |
| 669 | Anti-B7-H3 | EVQLVQSGAEVKKPGSSVKVSCKASGYSFTSYTIHWVRQAPGQGLEWMGYINPNSRNTDY |
| | VH | AQKFQGRVTLTADKSTSTAYMELSSLRSEDTAVYYCARYSGSTPYWYFDVWGQGTTVTVS |
| | | S |
| 670 | Anti-B7-H3 | DIQLTQSPSFLSASVGDRVTITCRASSSVSYMNWYQQKPGKSPKPWIYATSNLASGVPSR |
| | VL | FSVSVSGTEHTLTISSLQPEDFATYYCQQWSSNPLTFGQGTKLEIK |
| 671 | Anti-B7-H3 | SYWMH |
| | CDR-H1 | |
| 672 | Anti-B7-H3 | LIHPDSGSTNYNEMFKN |
| | CDR-H2 | |
| 673 | Anti-B7-H3 | GGRLYFDY |
| | CDR-H3 | |
| 674 | Anti-B7-H3 | RSSQSLVHSNGDTYLR |
| | CDR-L1 | |
| 675 | Anti-B7-H3 | KVSNRFS |
| | CDR-L2 | |
| 676 | Anti-B7-H3 | SQSTHVPYT |
| | CDR-L3 | |
| 677 | Anti-B7-H3 | EVQLVQSGAEVKKPGSSVKVSCKASGYTESSYWMHWVRQAPGQGLEWIGLIHPDSGSTNY |
| | VH | NEMFKNRATLTVDRSTSTAYVELSSLRSEDTAVYFCAGGGRLYFDYWGQGTTVTVSS |
| 678 | Anti-B7-H3 | DVVMTQSPLSLPVTPGEPASISCRSSQSLVHSNGDTYLRWYLQKPGQSPQLLIYKVSNRF |
| | VL | SGVPDRFSGSGSGTDFTLKISRVEAEDVGVYYCSQSTHVPYTFGGGTKVEIK |
| 679 | Anti-B7-H3 | SYWMH |
| | CDR-H1 | |
| 680 | Anti-B7-H3 | LIHPESGSTNYNEMFKN |
| | CDR-H2 | |
| 681 | Anti-B7-H3 | GGRLYFDY |
| | CDR-H3 | |
| 682 | Anti-B7-H3 | RSSQSLVHSNQDTYLR |
| | CDR-L1 | |
| 683 | Anti-B7-H3 | KVSNRFS |
| | CDR-L2 | |
| 684 | Anti-B7-H3 | SQSTHVPYT |
| | CDR-L3 | |
| 685 | Anti-B7-H3 | EVQLVQSGAEVKKPGSSVKVSCKASGYTFSSYWMHWVRQAPGQGLEWIGLIHPESGSTNY |
| | VH | NEMFKNRATLTVDRSTSTAYMELSSLRSEDTAVYYCAGGGRLYFDYWGQGTTVTVSS |
| 686 | Anti-B7-H3 | DIVMTQSPLSLPVTPGEPASISCRSSQSLVHSNQDTYLRWYLQKPGQSPQLLIYKVSNRF |
| | VL | SGVPDRFSGSGSGTDFTLKISRVEAEDVGVYYCSQSTHVPYTFGGGTKVEIK |
| 687 | Anti-B7-H3 | SGYSWH |
| | CDR-H1 | |
| 688 | Anti-B7-H3 | YIHSSGSTNYNPSLKS |
| | CDR-H2 | |
| 689 | Anti-B7-H3 | YDDYFEY |
| | CDR-H3 | |
| 690 | Anti-B7-H3 | KASQNVGENVAW |
| | CDR-L1 | |
| 691 | Anti-B7-H3 | SASYRYS |
| | CDR-L2 | |
| 692 | Anti-B7-H3 | QQYNWYPFT |
| | CDR-L3 | |
| 693 | Anti-B7-H3 | EVQLQESGPGLVKPSETLSLTCAVTGYSITSGYSWHWIRQFPGNGLEWMGYIHSSGSTNY |
| | VH | NPSLKSRISISRDTSKNQFFLKLSSVTAADTAVYYCAGYDDYFEYWGQGTTVTVSS |
| 694 | Anti-B7-H3 | DIQMTQSPSSLSASVGDRVTITCKASQNVGENVAWYQQKPGKSPKALIYSASYRYSGVPS |
| | VL | RFSGSGSGTDFTLTISSLQPEDFAEYFCQQYNWYPFTFGQGTKLEIK |
| 695 | Anti-B7-H3 | NYDIN |
| | CDR-H1 | |
| 696 | Anti-B7-H3 | WIFPGDDSTQYNEKFKG |
| | CDR-H2 | |
| 697 | Anti-B7-H3 | QTTGTWFAY |
| | CDR-H3 | |
| 698 | Anti-B7-H3 | RASQSISDYLY |
| | CDR-L1 | |
| 699 | Anti-B7-H3 | YASQSIS |
| | CDR-L2 | |
| 700 | Anti-B7-H3 | QNGHSFPLT |
| | CDR-L3 | |
| 701 | Anti-B7-H3 | QVQLVQSGAEVVKPGASVKLSCKTSGYTFTNYDINWVRQRPGQGLEWIGWIFPGDDSTQY |
| | VH | NEKFKGKATLTTDTSTSTAYMELSSLRSEDTAVYFCARQTTGTWFAYWGQGTLVTVSS |
| 702 | Anti-B7-H3 | EIVMTQSPATLSVSPGERVTLSCRASQSISDYLYWYQQKSHESPRLLIKYASQSISGIPA |
| | VL | RFSGSGSGSEFTLTINSVEPEDVGVYYCQNGHSFPLTFGQGTKLELK |
| 703 | Anti-B7-H3 | QVQLQQSGAEVKKPGSSVKVSCKASGGTFSSYAISWVRQAPGQGLEWMGGIIPILGIANY |
| | VH | AQKFQGRVTITADESTSTAYMELSSLRSEDTAVYYCARGGSGSYHMDVWGKGTTVTVSS |
| 704 | Anti-B7-H3 | EIVLTQSPATLSLSPGERATLSCRASQSVSSYLAWYQQKPGQAPRLLIYDASNRATGIPA |
| | VL | RFSGSGSGTDFTLTISSLEPEDFAVYYCQQRSNWPPRITFGQGTRLEIK |
| 705 | Anti-B7-H3 | IYNVH |
| | CDR-H1 | |
| 706 | Anti-B7-H3 | TIFPGNGDTSYNQKFKD |
| | CDR-H2 | |
| 707 | Anti-B7-H3 | WDDGNVGFAH |
| | CDR-H3 | |
| 708 | Anti-B7-H3 | RASENINNYLT |
| | CDR-L1 | |
| 709 | Anti-B7-H3 | HAKTLAE |
| | CDR-L2 | |
| 710 | Anti-B7-H3 | QHHYGTPPT |
| | CDR-L3 | |
| 711 | Anti-B7-H3 | QVQLQQPGAELVKPGASVKMSCKASGYTFTIYNVHWIKQTPGQGLEWMGTIFPGNGDTSY |
| | VH | NQKFKDKATLTTDKSSKTAYMQLNSLTSEDSAVYYCARWDDGNVGFAHWGQGTLVTVSA |
| 712 | Anti-B7-H3 | DIQMTQSPASLSASVGETVTITCRASENINNYLTWFQQKQGKSPQLLVYHAKTLAEGVPS |
| | VL | RFSGSGSGTQFSLKINSLQPEDFGSYYCQHHYGTPPTFGGGTKLEIK |
| 713 | Anti-B7-H3 | EVQLVQSGAEVKKPGASVKVSCKASGYTFTIYNVHWVRQAPGQGLEWMGTIFPGNGDTSY |
| | VH | NQKFKDKVTMTTDTSTSTAYMELSSLRSEDTAVYYCARWDDGNVGFAHWGQGTLVTVSS |
| 714 | Anti-B7-H3 | DIQMTQSPSSLSASVGDRVTITCRASENINNYLTWFQQKQGKSPQLLIYHAKTLAEGVPS |
| | VL | RFSGSGSGTDFTLTISSLQPEDFATYYCQHHYGTPPTFGGGTKVEIK |
| 715 | Anti-B7-H3 | EVQLVQSGAEVKKPGASVKVSCKASGYTFTIYNVHWIRQAPGQGLEWMGTIFPGNGDTSY |
| | VH | NQKFKDRATLTTDKSTKTAYMELRSLRSDDTAVYYCARWDDGNVGFAHWGQGTLVTVSS |
| 716 | Anti-B7-H3 | DIQMTQSPSSLSASVGDRVTITCRASENINNYLTWFQQKPGKAPKLLVYHAKTLAEGVPS |
| | VL | RFSGSGSGTQFTLTISSLQPEDFATYYCQHHYGTPPTFGQGTKLEIK |
| 717 | Anti-HER3 H | QVQLQQWGAGLLKPSETLSLTCAVYGGSFSGYYWSWIRQPPGKGLEWIGEINHSGSTNYN |
| | | PSLKSRVTISVETSKNQFSLKLSSVTAADTAVYYCARDKWTWYFDLWGRGTLVTVSSAST |
| | | KGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKRVEPKSCDKTHTCPPCPAPELLGGPSV |
| | | FLFPPKPKDTLMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLYSKLTVDKSRWQQG |
| | | NVFSCSVMHEALHNHYTQKSLSLSPGK |
| 718 | Anti-HER3 L | DIEMTQSPDSLAVSLGERATINCRSSQSVLYSSSNRNYLAWYQQNPGQPPKLLIYWASTR |
| | | ESGVPDRFSGSGSGTDFTLTISSLQAEDVAVYYCQQYYSTPRTFGQGTKVEIKRTVAAPS |
| | | VFIFPPSDEQLKSGTASVVCLLNNFYPREAKVQWKVDNALQSGNSQESVTEQDSKDSTYS |
| | | LSSTLTLSKADYEKHKVYACEVTHQGLSSPVTKSFNRGEC |
| 719 | Anti-HER3 H | EVQLLESGGGLVQPGGSLRLSCAASGFTFSHYVMAWVRQAPGKGLEWVSSISSSGGWTLY |
| | | ADSVKGRFTISRDNSKNTLYLQMNSLRAEDTAVYYCTRGLKMATIFDYWGQGTLVTVSSA |
| | | STKGPSVFPLAPCSRSTSESTAALGCLVKDYFPEPVTVSWNSGALTSGVHTFPAVLQSSG |
| | | LYSLSSVVTVPSSNFGTQTYTCNVDHKPSNTKVDKTVERKCCVECPPCPAPPVAGPSVFL |
| | | FPPKPKDTLMISRTPEVTCVVVDVSHEDPEVQFNWYVDGVEVHNAKTKPREEQFNSTFRV |
| | | VSVLTVVHQDWLNGKEYKCKVSNKGLPAPIEKTISKTKGQPREPQVYTLPPSREEMTKNQ |
| | | VSLTCLVKGFYPSDIAVEWESNGQPENNYKTTPPMLDSDGSFFLYSKLTVDKSRWQQGNV |
| | | FSCSVMHEALHNHYTQKSLSLSPGK |
| 720 | Anti-HER3 L | QSALTQPASVSGSPGQSITISCTGTSSDVGSYNVVSWYQQHPGKAPKLIIYEVSQRPSGV |
| | | SNRFSGSKSGNTASLTISGLQTEDEADYYCCSYAGSSIFVIFGGGTKVTVLGQPKAAPSV |
| | | TLFPPSSEELQANKATLVCLVSDFYPGAVTVAWKADGSPVKVGVETTKPSKQSNNKYAAS |
| | | SYLSLTPEQWKSHRSYSCRVTHEGSTVEKTVAPAECS |
| 721 | Anti-HER3 H | EVQLLESGGGLVQPGGSLRLSCAASGFTFSSYAMSWVRQAPGKGLEWVSAINSQGKSTYY |
| | | ADSVKGRFTISRDNSKNTLYLQMNSLRAEDTAVYYCARWGDEGFDIWGQGTLVTVSSAST |
| | | KGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKRVEPKSCDKTHTCPPCPAPELLGGPSV |
| | | FLFPPKPKDTLMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYTLPPSREEMTK |
| | | NQVSLTCLVKGFYPSDIAVEWESNGQPENNYKTTPPVLDSDGSFFLYSKLTVDKSRWQQG |
| | | NVFSCSVMHEALHNHYTQKSLSLSPGK |
| 722 | Anti-HER3 L | DIQMTQSPSSLSASVGDRVTITCRASQGISNWLAWYQQKPGKAPKLLIYGASSLQSGVPS |
| | | RFSGSGSGTDFTLTISSLQPEDFATYYCQQYSSFPTTFGQGTKVEIKRTVAAPSVFIFPP |
| | | SDEQLKSGTASVVCLLNNFYPREAKVQWKVDNALQSGNSQESVTEQDSKDSTYSLSSTLT |
| | | LSKADYEKHKVYACEVTHQGLSSPVTKSENRGEC |
| 723 | Anti-HER3 H | QVQLVQSGAEVKKPGASVKVSCKASGYTFRSSYISWVRQAPGQGLEWMGWIYAGTGSPSY |
| | | NQKLQGRVTMTTDTSTSTAYMELRSLRSDDTAVYYCARHRDYYSNSLTYWGQGTLVTVSS |
| | | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPG |
| 724 | Anti-HER3 L | DIVMTQSPDSLAVSLGERATINCKSSQSVLNSGNQKNYLTWYQQKPGQPPKLLIYWASTR |
| | | ESGVPDRFSGSGSGTDFTLTISSLQAEDVAVYYCQSDYSYPYTFGQGTKLEIKRTVAAPS |
| | | VFIFPPSDEQLKSGTASVVCLLNNFYPREAKVQWKVDNALQSGNSQESVTEQDSKDSTYS |
| | | LSSTLTLSKADYEKHKVYACEVTHQGLSSPVTKSFNRGEC |
| 725 | Anti-PTK7 | TSNMGVG |
| | CDR-H1 | |
| 726 | Anti-PTK7 | HIWWDDDKYYSPSLKS |
| | CDR-H2 | |
| 727 | Anti-PTK7 | SNYGYAWFAY |
| | CDR-H3 | |
| 728 | Anti-PTK7 | KASQDIYPYLN |
| | CDR-L1 | |
| 729 | Anti-PTK7 | RTNRLLD |
| | CDR-L2 | |
| 730 | Anti-PTK7 | LQYDEFPLT |
| | CDR-L3 | |
| 731 | Anti-PTK7 | QITLKESGPTLVKPTQTLTLTCTFSGFSLSTSNMGVGWIRQPPGKALEWLAHIWWDDDKY |
| | VH | YSPSLKSRLTITKDTSKNQVVLTMTNMDPVDTATYYCVRSNYGYAWFAYWGQGTLVTVSS |
| 732 | Anti-PTK7 | DIQMTQSPSSLSASVGDRVTITCKASQDIYPYLNWFQQKPGKAPKTLIYRTNRLLDGVPS |
| | VL | RFSGSGSGTDFTFTISSLQPEDIATYYCLQYDEFPLTFGAGTKLEIK |
| 733 | Anti-PTK7 | DYAVH |
| | CDR-H1 | |
| 734 | Anti-PTK7 | VISTYNDYTYNNQDFKG |
| | CDR-H2 | |
| 735 | Anti-PTK7 | GNSYFYALDY |
| | CDR-H3 | |
| 736 | Anti-PTK7 | RASESVDSYGKSFMH |
| | CDR-L1 | |
| 737 | Anti-PTK7 | RASNLES |
| | CDR-L2 | |
| 738 | Anti-PTK7 | QQSNEDPWT |
| | CDR-L3 | |
| 739 | Anti-PTK7 | QVQLVQSGPEVKKPGASVKVSCKASGYTFTDYAVHWVRQAPGKRLEWIGVISTYNDYTYN |
| | VH | NQDFKGRVTMTRDTSASTAYMELSRLRSEDTAVYYCARGNSYFYALDYWGQGTSVTVSS |
| 740 | Anti-PTK7 | EIVLTQSPATLSLSPGERATLSCRASESVDSYGKSFMHWYQQKPGQAPRLLIYRASNLES |
| | VL | GIPARFSGSGSGTDFTLTISSLEPEDFAVYYCQQSNEDPWTFGGGTKLEIK |
| 741 | Anti-PTK7 | RYWMS |
| | CDR-H1 | |
| 742 | Anti-PTK7 | DLNPDSSAINYVDSVKG |
| | CDR-H2 | |
| 743 | Anti-PTK7 | ITTLVPYTMDF |
| | CDR-H3 | |
| 744 | Anti-PTK7 | ITNTDIDDDMN |
| | CDR-L1 | |
| 745 | Anti-PTK7 | EGNGLRP |
| | CDR-L2 | |
| 746 | Anti-PTK7 | LQSDNLPLT |
| | CDR-L3 | |
| 747 | Anti-PTK7 | EVQLVESGGGLVQPGGSLRLSCAASGFDFSRYWMSWVRQAPGKGLEWIGDLNPDSSAINY |
| | VH | VDSVKGRFTISRDNAKNSLYLQMNSLRAEDTAVYYCTLITTLVPYTMDFWGQGTSVTVSS |
| 748 | Anti-PTK7 | ETTLTQSPAFMSATPGDKVNISCITNTDIDDDMNWYQQKPGEAAILLISEGNGLRPGIPP |
| | VL | RFSGSGYGTDFTLTINNIESEDAAYYFCLQSDNLPLTFGSGTKLEIK |
| 749 | Anti-ZIP6 | DYYMH |
| | CDR-H1 | |
| 750 | Anti-ZIP6 | WIDPENGDTEYGPKFQG |
| | CDR-H2 | |
| 751 | Anti-ZIP6 | HNAHYGTWFAY |
| | CDR-H3 | |
| 752 | Anti-ZIP6 | RSSQSLLHSSGNTYLE |
| | CDR-L1 | |
| 753 | Anti-ZIP6 | KISTRES |
| | CDR-L2 | |
| 754 | Anti-ZIP6 | FQGSHVPYT |
| | CDR-L3 | |
| 755 | Anti-ZIP6 | QVQLVQSGAEVKKPGASVKVSCKASGLTIEDYYMHWVRQAPGQGLEWMGWIDPENGDTEY |
| | VH | GPKFQGRVTMTRDTSINTAYMELSRLRSDDTAVYYCAVHNAHYGTWFAYWGQGTLVTVSS |
| 756 | Anti-ZIP6 | DVVMTQSPLSLPVTLGQPASISCRSSQSLLHSSGNTYLEWYQQRPGQSPRPLIYKISTRF |
| | VL | SGVPDRFSGSGSGTDFTLKISRVEAEDVGVYYCFQGSHVPYTFGGGTKVEIK |
| 757 | Anti-ZIP6 | QVQLVQSGAEVKKPGASVKVSCKASGLTIEDYYMHWVRQAPGQGLEWMGWIDPENGDTEY |
| | HC | GPKFQGRVTMTRDTSINTAYMELSRLRSDDTAVYYCAVHNAHYGTWFAYWGQGTLVTVSS |
| | | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFFLYSKLTVDKSRW |
| | | QQGNVFSCSVMHEALHNHYTQKSLSLSPG |
| 758 | Anti-ZIP6 | DVVMTQSPLSLPVTLGQPASISCRSSQSLLHSSGNTYLEWYQQRPGQSPRPLIYKISTRF |
| | LC | SGVPDRFSGSGSGTDFTLKISRVEAEDVGVYYCFQGSHVPYTFGGGTKVEIKRTVAAPSV |
| | | FIFPPSDEQLKSGTASVVCLLNNFYPREAKVQWKVDNALQSGNSQESVTEQDSKDSTYSL |
| | | SSTLTLSKADYEKHKVYACEVTHQGLSSPVTKSENRGEC |
| 759 | Anti- | GYFMN |
| | Integrin | |
| | αvβ6 CDR-H1 | |
| 760 | Anti- | LINPYNGDSFYNQKFKG |
| | Integrin | |
| | αvβ6 CDR-H2 | |
| 761 | Anti- | GLRRDFDY |
| | Integrin | |
| | αvß6 CDR-H3 | |
| 762 | Anti- | KSSQSLLDSDGKTYLN |
| | Integrin | |
| | αvβ6 CDR-L1 | |
| 763 | Anti- | LVSELDS |
| | Integrin | |
| | αvß6 CDR-L2 | |
| 764 | Anti- | WQGTHFPRT |
| | Integrin | |
| | αvβ6 CDR-L3 | |
| 765 | Anti- | QVQLVQSGAEVKKPGASVKVSCKASGYSFSGYFMNWVRQAPGQGLEWMGLINPYNGDSFY |
| | Integrin | NQKFKGRVTMTRQTSTSTVYMELSSLRSEDTAVYYCVRGLRRDFDYWGQGTLVTVSS |
| | αvß6 VH | |
| 766 | Anti- | DVVMTQSPLSLPVTLGQPASISCKSSQSLLDSDGKTYLNWLFQRPGQSPRRLIYLVSELD |
| | Integrin | SGVPDRFSGSGSGTDFTLKISRVEAEDVGVYYCWQGTHFPRTFGGGTKLEIK |
| | αvβ6 VL | |
| 767 | Anti-CD48 | DFGMN |
| | CDR-H1 | |
| 768 | Anti-CD48 | WINTFTGEPSYGNVFKG |
| | CDR-H2 | |
| 769 | Anti-CD48 | RHGNGNVEDS |
| | CDR-H3 | |
| 770 | Anti-CD48 | RASQSIGSNIH |
| | CDR-L1 | |
| 771 | Anti-CD48 | YTSESIS |
| | CDR-L2 | |
| 772 | Anti-CD48 | QQSNSWPLT |
| | CDR-L3 | |
| 773 | Anti-CD48 | QVQLVQSGSELKKPGASVKVSCKASGYTFTDFGMNWVRQAPGQGLEWMGWINTFTGEPSY |
| | VH | GNVFKGRFVFSLDTSVSTAYLQISSLKAEDTAVYYCARRHGNGNVFDSWGQGTLVTVSS |
| 774 | Anti-CD48 | EIVLTQSPDFQSVTPKEKVTITCRASQSIGSNIHWYQQKPDQSPKLLIKYTSESISGVPS |
| | VL | RFSGSGSGTDFTLTINSLEAEDAATYYCQQSNSWPLTFGGGTKVEIKR |
| 775 | Anti-IGF-1R | SYAIS |
| | CDR-H1 | |
| 776 | Anti-IGF-1R | GIIPIFGTANYAQKFQG |
| | CDR-H2 | |
| 777 | Anti-IGF-1R | APLRFLEWSTQDHYYYYYMDV |
| | CDR-H3 | |
| 778 | Anti-IGF-1R | QGDSLRSYYAT |
| | CDR-L1 | |
| 779 | Anti-IGF-1R | GENKRPS |
| | CDR-L2 | |
| 780 | Anti-IGF-1R | KSRDGSGQHLV |
| | CDR-L3 | |
| 781 | Anti-IGF-1R | EVQLVQSGAEVKKPGSSVKVSCKASGGTFSSYAISWVRQAPGQGLEWMGGIIPIFGTANY |
| | VH | AQKFQGRVTITADKSTSTAYMELSSLRSEDTAVYYCARAPLRFLEWSTQDHYYYYYMDVW |
| | | GKGTTVTVSS |
| 782 | Anti-IGF-1R | SSELTQDPAVSVALGQTVRITCQGDSLRSYYATWYQQKPGQAPILVIYGENKRPSGIPDR |
| | VL | FSGSSSGNTASLTITGAQAEDEADYYCKSRDGSGQHLVFGGGTKLTVL |
| 783 | Anti- | SYWIN |
| | Claudin- | |
| | 18.2 CDR-H1 | |
| 784 | Anti- | NIYPSDSYTNYNQKFKD |
| | Claudin- | |
| | 18.2 CDR-H2 | |
| 785 | Anti- | SWRGNSFDY |
| | Claudin- | |
| | 18.2 CDR-H3 | |
| 786 | Anti- | KSSQSLLNSGNQKNYLT |
| | Claudin- | |
| | 18.2 CDR-L1 | |
| 787 | Anti- | WASTRES |
| | Claudin- | |
| | 18.2 CDR-L2 | |
| 788 | Anti- | QNDYSYPFT |
| | Claudin- | |
| | 18.2 CDR-L3 | |
| 789 | Anti- | QVQLQQPGAELVRPGASVKLSCKASGYTFTSYWINWVKQRPGQGLEWIGNIYPSDSYTNY |
| | Claudin- | NQKFKDKATLTVDKSSSTAYMQLSSPTSEDSAVYYCTRSWRGNSFDYWGQGTTLTVSS |
| | 18.2 VH | |
| 790 | Anti- | DIVMTQSPSSLTVTAGEKVTMSCKSSQSLLNSGNQKNYLTWYQQKPGQPPKLLIYWASTR |
| | Claudin- | ESGVPDRFTGSGSGTDFTLTISSVQAEDLAVYYCQNDYSYPFTFGSGTKLEIK |
| | 18.2 VL | |
| 791 | Anti- | NYGMN |
| | Claudin- | |
| | 18.2 CDR-H1 | |
| 792 | Anti- | WINTNTGEPTYAEEFKG |
| | Claudin- | |
| | 18.2 CDR-H2 | |
| 793 | Anti- | LGFGNAMDY |
| | Claudin- | |
| | 18.2 CDR-H3 | |
| 794 | Anti- | KSSQSLLNSGNQKNYLT |
| | Claudin- | |
| | 18.2 CDR-L1 | |
| 795 | Anti- | WASTRES |
| | Claudin- | |
| | 18.2 CDR-L2 | |
| 796 | Anti- | QNDYSYPLT |
| | Claudin- | |
| | 18.2 CDR-L3 | |
| 797 | Anti- | QIQLVQSGPELKKPGETVKISCKASGYTFTNYGMNWVKQAPGKGLKWMGWINTNTGEPTY |
| | Claudin- | AEEFKGRFAFSLETSASTAYLQINNLKNEDTATYFCARLGFGNAMDYWGQGTSVTVSS |
| | 18.2 VH | |
| 798 | Anti- | DIVMTQSPSSLTVTAGEKVTMSCKSSQSLLNSGNQKNYLTWYQQKPGQPPKLLIYWASTR |
| | Claudin- | ESGVPDRFTGSGSGTDFTLTISSVQAEDLAVYYCQNDYSYPLTFGAGTKLELK |
| | 18.2 VL | |
| 799 | Anti- | SYNMN |
| | Nectin-4 | |
| | CDR-H1 | |
| 800 | Anti- | YISSSSSTIYYADSVKG |
| | Nectin-4 | |
| | CDR-H2 | |
| 801 | Anti- | AYYYGMDV |
| | Nectin-4 | |
| | CDR-H3 | |
| 802 | Anti- | RASQGISGWLA |
| | Nectin-4 | |
| | CDR-L1 | |
| 803 | Anti- | AASTLQS |
| | Nectin-4 | |
| | CDR-L2 | |
| 804 | Anti- | QQANSFPPT |
| | Nectin-4 | |
| | CDR-L3 | |
| 805 | Anti- | EVQLVESGGGLVQPGGSLRLSCAASGFTFSSYNMNWVRQAPGKGLEWVSYISSSSSTIYY |
| | Nectin-4 VH | ADSVKGRFTISRDNAKNSLSLQMNSLRDEDTAVYYCARAYYYGMDVWGQGTTVTVSS |
| 806 | Anti- | DIQMTQSPSSVSASVGDRVTITCRASQGISGWLAWYQQKPGKAPKFLIYAASTLQSGVPS |
| | Nectin-4 VL | RFSGSGSGTDFTLTISSLQPEDFATYYCQQANSFPPTFGGGTKVEIK |
| 807 | Anti-SLTRK6 | SYGMH |
| | CDR-H1 | |
| 808 | Anti-SLTRK6 | VIWYDGSNQYYADSVKG |
| | CDR-H2 | |
| 809 | Anti-SLTRK6 | GLTSGRYGMDV |
| | CDR-H3 | |
| 810 | Anti-SLTRK6 | RSSQSLLLSHGFNYLD |
| | CDR-L1 | |
| 811 | Anti-SLTRK6 | LGSSRAS |
| | CDR-L2 | |
| 812 | Anti-SLTRK6 | MQPLQIPWT |
| | CDR-L3 | |
| 813 | Anti-SLTRK6 | QVQLVESGGGVVQPGRSLRLSCAASGFTFSSYGMHWVRQAPGKGLEWVAVIWYDGSNQYY |
| | VH | ADSVKGRFTISRDNSKNTLFLQMHSLRAEDTAVYYCARGLTSGRYGMDVWGQGTTVTVSS |
| 814 | Anti-SLTRK6 | DIVMTQSPLSLPVTPGEPASISCRSSQSLLLSHGFNYLDWYLQKPGQSPQLLIYLGSSRA |
| | VL | SGVPDRFSGSGSGTDFTLKISRVEAEDVGLYYCMQPLQIPWTFGQGTKVEIK |
| 815 | Anti-CD142 | NYAMS |
| | (TF) CDR-H1 | |
| 816 | Anti-CD142 | SISGSGDYTYYTDSVKG |
| | (TF) CDR-H2 | |
| 817 | Anti-CD142 | SPWGYYLDS |
| | (TF) CDR-H3 | |
| 818 | Anti-CD142 | RASQGISSRLA |
| | (TF) CDR-L1 | |
| 819 | Anti-CD142 | AASSLQS |
| | (TF) CDR-L2 | |
| 820 | Anti-CD142 | QQYNSYPYT |
| | (TF) CDR-L3 | |
| 821 | Anti-CD142 | EVQLLESGGGLVQPGGSLRLSCAASGFTFSNYAMSWVRQAPGKGLEWVSSISGSGDYTYY |
| | (TF) VH | TDSVKGRFTISRDNSKNTLYLQMNSLRAEDTAVYYCARSPWGYYLDSWGQGTLVTVSS |
| 822 | Anti-CD142 | DIQMTQSPPSLSASAGDRVTITCRASQGISSRLAWYQQKPEKAPKSLIYAASSLQSGVPS |
| | (TF) VL | RFSGSGSGTDFTLTISSLQPEDFATYYCQQYNSYPYTFGQGTKLEIK |
| 823 | Anti- | DHAIH |
| | h2G12/STn | |
| | CDR-H1 | |
| 824 | Anti-STn | YFSPGNDDIKYNEKFRG |
| | CDR-H2 | |
| 825 | Anti-STn | SLSTPY |
| | CDR-H3 | |
| 826 | Anti-STn | KSSQSLLNRGNHKNYLT |
| | CDR-L1 | |
| 827 | Anti-STn | WASTRES |
| | CDR-L2 | |
| 828 | Anti-STn | QNDYTYPYT |
| | CDR-L3 | |
| 829 | Anti-STn VH | EVQLVQSGAEVKKPGASVKVSCKASGYTFTDHAIHWVRQAPGQGLEWMGYFSPGNDDIKY |
| | | NEKFRGRVTMTADKSSSTAYMELRSLRSDDTAVYFCKRSLSTPYWGQGTLVTVSS |
| 830 | Anti-STn VL | DIVMTQSPDSLAVSLGERATINCKSSQSLLNRGNHKNYLTWYQQKPGQPPKLLIYWASTR |
| | | ESGVPDRFSGSGSGTDFTLTISSLQAEDVAVYYCQNDYTYPYTFGQGTKVEIK |
| 831 | Anti-CD20 | SYNMH |
| | CDR-H1 | |
| 832 | Anti-CD20 | AIYPGNGDTSYNQKFKG |
| | CDR-H2 | |
| 833 | Anti-CD20 | STYYGGDWYFNV |
| | CDR-H3 | |
| 834 | Anti-CD20 | RASSSVSYIH |
| | CDR-L1 | |
| 835 | Anti-CD20 | ATSNLAS |
| | CDR-L2 | |
| 836 | Anti-CD20 | QQWTSNPPT |
| | CDR-L3 | |
| 837 | Anti-CD20 | QVQLQQPGAELVKPGASVKMSCKASGYTFTSYNMHWVKQTPGRGLEWIGAIYPGNGDTSY |
| | VH | NQKFKGKATLTADKSSSTAYMQLSSLTSEDSAVYYCARSTYYGGDWYFNVWGAGTTVTVS |
| | | A |
| 838 | Anti-CD20 | QIVLSQSPAILSASPGEKVTMTCRASSSVSYIHWFQQKPGSSPKPWIYATSNLASGVPVR |
| | VL | FSGSGSGTSYSLTISRVEAEDAATYYCQQWTSNPPTFGGGTKLEIK |
| 839 | Anti-HER2 | DTYIH |
| | CDR-H1 | |
| 840 | Anti-HER2 | RIYPTNGYTRYADSVKG |
| | CDR-H2 | |
| 841 | Anti-HER2 | WGGDGFYAMDY |
| | CDR-H3 | |
| 842 | Anti-HER2 | RASQDVNTAVA |
| | CDR-L1 | |
| 843 | Anti-HER2 | SASFLYS |
| | CDR-L2 | |
| 844 | Anti-HER2 | QQHYTTPPT |
| | CDR-L3 | |
| 845 | Anti-HER2 | EVQLVESGGGLVQPGGSLRLSCAASGFNIKDTYIHWVRQAPGKGLEWVARIYPTNGYTRY |
| | VH | ADSVKGRFTISADTSKNTAYLQMNSLRAEDTAVYYCSRWGGDGFYAMDYWGQGTLVTVSS |
| 846 | Anti-HER2 | DIQMTQSPSSLSASVGDRVTITCRASQDVNTAVAWYQQKPGKAPKLLIYSASFLYSGVPS |
| | VL | RFSGSRSGTDFTLTISSLQPEDFATYYCQQHYTTPPTFGQGTKVEIK |
| 847 | Anti-CD79b | SYWIE |
| | CDR-H1 | |
| 848 | Anti-CD79b | EILPGGGDTNYNEIFKG |
| | CDR-H2 | |
| 849 | Anti-CD79b | RVPIRLDY |
| | CDR-H3 | |
| 850 | Anti-CD79b | KASQSVDYEGDSFLN |
| | CDR-L1 | |
| 851 | Anti-CD79b | AASNLES |
| | CDR-L2 | |
| 852 | Anti-CD79b | QQSNEDPLT |
| | CDR-L3 | |
| 853 | Anti-CD79b | EVQLVESGGGLVQPGGSLRLSCAASGYTFSSYWIEWVRQAPGKGLEWIGEILPGGGDTNY |
| | VH | NEIFKGRATFSADTSKNTAYLQMNSLRAEDTAVYYCTRRVPIRLDYWGQGTLVTVSS |
| 854 | Anti-CD79b | DIQLTQSPSSLSASVGDRVTITCKASQSVDYEGDSFLNWYQQKPGKAPKLLIYAASNLES |
| | VL | GVPSRFSGSGSGTDFTLTISSLQPEDFATYYCQQSNEDPLTFGQGTKVEIK |
| 855 | Anti-NaPi2B | DFAMS |
| | CDR-H1 | |
| 856 | Anti-NaPi2B | TIGRVAFHTYYPDSMKG |
| | CDR-H2 | |
| 857 | Anti-NaPi2B | HRGFDVGHFDF |
| | CDR-H3 | |
| 858 | Anti-NaPi2B | RSSETLVHSSGNTYLE |
| | CDR-L1 | |
| 859 | Anti-NaPi2B | RVSNRFS |
| | CDR-L2 | |
| 860 | Anti-NaPi2B | FQGSFNPLT |
| | CDR-L3 | |
| 861 | Anti-NaPi2B | EVQLVESGGGLVQPGGSLRLSCAASGFSFSDFAMSWVRQAPGKGLEWVATIGRVAFHTYY |
| | VH | PDSMKGRFTISRDNSKNTLYLQMNSLRAEDTAVYYCARHRGFDVGHFDFWGQGTLVTVSS |
| 862 | Anti-NaPi2B | DIQMTQSPSSLSASVGDRVTITCRSSETLVHSSGNTYLEWYQQKPGKAPKLLIYRVSNRF |
| | VL | SGVPSRFSGSGSGTDFTLTISSLQPEDFATYYCFQGSFNPLTFGQGTKVEIK |
| 863 | Anti-Muc16 | NDYAWN |
| | CDR-H1 | |
| 864 | Anti-Muc16 | YISYSGYTTYNPSLKS |
| | CDR-H2 | |
| 865 | Anti-Muc16 | WTSGLDY |
| | CDR-H3 | |
| 866 | Anti-Muc16 | KASDLIHNWLA |
| | CDR-L1 | |
| 867 | Anti-Muc16 | GATSLET |
| | CDR-L2 | |
| 868 | Anti-Muc16 | QQYWTTPFT |
| | CDR-L3 | |
| 869 | Anti-Muc16 | EVQLVESGGGLVQPGGSLRLSCAASGYSITNDYAWNWVRQAPGKGLEWVGYISYSGYTTY |
| | VH | NPSLKSRFTISRDTSKNTLYLQMNSLRAEDTAVYYCARWTSGLDYWGQGTLVTVSS |
| 870 | Anti-Muc16 | DIQMTQSPSSLSASVGDRVTITCKASDLIHNWLAWYQQKPGKAPKLLIYGATSLETGVPS |
| | VL | RFSGSGSGTDFTLTISSLQPEDFATYYCQQYWTTPFTFGQGTKVEIK |
| 871 | Anti-STEAP1 | SDYAWN |
| | CDR-H1 | |
| 872 | Anti-STEAP1 | YISNSGSTSYNPSLKS |
| | CDR-H2 | |
| 873 | Anti-STEAP1 | ERNYDYDDYYYAMDY |
| | CDR-H3 | |
| 874 | Anti-STEAP1 | KSSQSLLYRSNQKNYLA |
| | CDR-L1 | |
| 875 | Anti-STEAP1 | WASTRES |
| | CDR-L2 | |
| 876 | Anti-STEAP1 | QQYYNYPRT |
| | CDR-L3 | |
| 877 | Anti-STEAP1 | EVQLVESGGGLVQPGGSLRLSCAVSGYSITSDYAWNWVRQAPGKGLEWVGYISNSGSTSY |
| | VH | NPSLKSRFTISRDTSKNTLYLQMNSLRAEDTAVYYCARERNYDYDDYYYAMDYWGQGTLV |
| | | TVSS |
| 878 | Anti-STEAP1 | DIQMTQSPSSLSASVGDRVTITCKSSQSLLYRSNQKNYLAWYQQKPGKAPKLLIYWASTR |
| | VL | ESGVPSRFSGSGSGTDFTLTISSLQPEDFATYYCQQYYNYPRTFGQGTKVEIK |
| 879 | Anti-BCMA | NYWMH |
| | CDR-H1 | |
| 880 | Anti-BCMA | ATYRGHSDTYYNQKFKG |
| | CDR-H2 | |
| 881 | Anti-BCMA | GAIYDGYDVLDN |
| | CDR-H3 | |
| 882 | Anti-BCMA | SASQDISNYLN |
| | CDR-L1 | |
| 883 | Anti-BCMA | YTSNLHS |
| | CDR-L2 | |
| 884 | Anti-BCMA | QQYRKLPWT |
| | CDR-L3 | |
| 885 | Anti-BCMA | QVQLVQSGAEVKKPGSSVKVSCKASGGTFSNYWMHWVRQAPGQGLEWMGATYRGHSDTYY |
| | VH | NQKFKGRVTITADKSTSTAYMELSSLRSEDTAVYYCARGAIYDGYDVLDNWGQGTLVTVS |
| | | S |
| 886 | Anti-BCMA | DIQMTQSPSSLSASVGDRVTITCSASQDISNYLNWYQQKPGKAPKLLIYYTSNLHSGVPS |
| | VL | RFSGSGSGTDFTLTISSLQPEDFATYYCQQYRKLPWTFGQGTKLEIK |
| 887 | Anti-c-Met | AYTMH |
| | CDR-H1 | |
| 888 | Anti-c-Met | WIKPNNGLANYAQKFQG |
| | CDR-H2 | |
| 889 | Anti-c-Met | SEITTEFDY |
| | CDR-H3 | |
| 890 | Anti-c-Met | KSSESVDSYANSFLH |
| | CDR-L1 | |
| 891 | Anti-c-Met | RASTRES |
| | CDR-L2 | |
| 892 | Anti-c-Met | QQSKEDPLT |
| | CDR-L3 | |
| 893 | Anti-c-Met | QVQLVQSGAEVKKPGASVKVSCKASGYIFTAYTMHWVRQAPGQGLEWMGWIKPNNGLANY |
| | VH | AQKFQGRVTMTRDTSISTAYMELSRLRSDDTAVYYCARSEITTEFDYWGQGTLVTVSS |
| 894 | Anti-c-Met | DIVMTQSPDSLAVSLGERATINCKSSESVDSYANSFLHWYQQKPGQPPKLLIYRASTRES |
| | VL | GVPDRFSGSGSGTDFTLTISSLQAEDVAVYYCQQSKEDPLTFGGGTKVEIK |
| 895 | Anti-EGFR | SDFAWN |
| | CDR-H1 | |
| 896 | Anti-EGFR | YISYSGNTRYQPSLKS |
| | CDR-H2 | |
| 897 | Anti-EGFR | AGRGFPY |
| | CDR-H3 | |
| 898 | Anti-EGFR | HSSQDINSNIG |
| | CDR-L1 | |
| 899 | Anti-EGFR | HGTNLDD |
| | CDR-L2 | |
| 900 | Anti-EGFR | VQYAQFPWT |
| | CDR-L3 | |
| 901 | Anti-EGFR | QVQLQESGPGLVKPSQTLSLTCTVSGYSISSDFAWNWIRQPPGKGLEWMGYISYSGNTRY |
| | VH | QPSLKSRITISRDTSKNQFFLKLNSVTAADTATYYCVTAGRGFPYWGQGTLVTVSS |
| 902 | Anti-EGFR | DIQMTQSPSSMSVSVGDRVTITCHSSQDINSNIGWLQQKPGKSFKGLIYHGTNLDDGVPS |
| | VL | RFSGSGSGTDYTLTISSLQPEDFATYYCVQYAQFPWTFGGGTKLEIK |
| 903 | Anti-SLAMF7 | DYYMA |
| | CDR-H1 | |
| 904 | Anti-SLAMF7 | SINYDGSSTYYVDSVKG |
| | CDR-H2 | |
| 905 | Anti-SLAMF7 | DRGYYFDY |
| | CDR-H3 | |
| 906 | Anti-SLAMF7 | RSSQSLVHSNGNTYLH |
| | CDR-L1 | |
| 907 | Anti-SLAMF7 | KVSNRFS |
| | CDR-L2 | |
| 908 | Anti-SLAMF7 | SQSTHVPPFT |
| | CDR-L3 | |
| 909 | Anti-SLAMF7 | EVQLVESGGGLVQPGGSLRLSCAASGFTFSDYYMAWVRQAPGKGLEWVASINYDGSSTYY |
| | VH | VDSVKGRFTISRDNAKNSLYLQMNSLRAEDTAVYYCARDRGYYFDYWGQGTTVTVSS |
| 910 | Anti-SLAMF7 | DVVMTQTPLSLSVTPGQPASISCRSSQSLVHSNGNTYLHWYLQKPGQSPQLLIYKVSNRF |
| | VL | SGVPDRFSGSGSGTDFTLKISRVEAEDVGVYFCSQSTHVPPFTFGGGTKVEIK |
| 911 | Anti-C4.4a | NAWMS |
| | CDR-H1 | |
| 912 | Anti-C4.4a | YISSSGSTIYYADSVKG |
| | CDR-H2 | |
| 913 | Anti-C4.4a | EGLWAFDY |
| | CDR-H3 | |
| 914 | Anti-C4.4a | TGSSSNIGAGYVVH |
| | CDR-L1 | |
| 915 | Anti-C4.4a | DNNKRPS |
| | CDR-L2 | |
| 916 | Anti-C4.4a | AAWDDRLNGPV |
| | CDR-L3 | |
| 917 | Anti-C4.4a | EVQLLESGGGLVQPGGSLRLSCAASGFTFSNAWMSWVRQAPGKGLEWVSYISSSGSTIYY |
| | VH | ADSVKGRFTISRDNSKNTLYLQMNSLRAEDTAVYYCAREGLWAFDYWGQGTLVTVSS |
| 918 | Anti-C4.4a | ESVLTQPPSVSGAPGQRVTISCTGSSSNIGAGYVVHWYQQLPGTAPKLLIYDNNKRPSGV |
| | VL | PDRFSGSKSGTSASLAISGLRSEDEADYYCAAWDDRLNGPVFGGGTKLTVL |
| 919 | Anti-GCC | GYYWS |
| | CDR-H1 | |
| 920 | Anti-GCC | EINHRGNTNDNPSLKS |
| | CDR-H2 | |
| 921 | Anti-GCC | ERGYTYGNFDH |
| | CDR-H3 | |
| 922 | Anti-GCC | RASQSVSRNLA |
| | CDR-L1 | |
| 923 | Anti-GCC | GASTRAT |
| | CDR-L2 | |
| 924 | Anti-GCC | QQYKTWPRT |
| | CDR-L3 | |
| 925 | Anti-GCC VH | QVQLQQWGAGLLKPSETLSLTCAVFGGSFSGYYWSWIRQPPGKGLEWIGEINHRGNTNDN |
| | | PSLKSRVTISVDTSKNQFALKLSSVTAADTAVYYCARERGYTYGNFDHWGQGTLVTVSS |
| 926 | Anti-GCC VL | EIVMTQSPATLSVSPGERATLSCRASQSVSRNLAWYQQKPGQAPRLLIYGASTRATGIPA |
| | | RFSGSGSGTEFTLTIGSLQSEDFAVYYCQQYKTWPRTFGQGTNVEIK |
| 927 | Anti-Axl | SYAMN |
| | CDR-H1 | |
| 928 | Anti-Axl | TTSGSGASTYYADSVKG |
| | CDR-H2 | |
| 929 | Anti-Axl | IWIAFDI |
| | CDR-H3 | |
| 930 | Anti-Axl | RASQSVSSSYLA |
| | CDR-L1 | |
| 931 | Anti-Axl | GASSRAT |
| | CDR-L2 | |
| 932 | Anti-Axl | QQYGSSPYT |
| | CDR-L3 | |
| 933 | Anti-Axl VH | EVQLLESGGGLVQPGGSLRLSCAASGFTFSSYAMNWVRQAPGKGLEWVSTTSGSGASTYY |
| | | ADSVKGRFTISRDNSKNTLYLQMNSLRAEDTAVYYCAKIWIAFDIWGQGTMVTVSS |
| 934 | Anti-Axl VL | EIVLTQSPGTLSLSPGERATLSCRASQSVSSSYLAWYQQKPGQAPRLLIYGASSRATGIP |
| | | DRFSGSGSGTDFTLTISRLEPEDFAVYYCQQYGSSPYTFGQGTKLEIK |
| 935 | Anti-gpNMB | SFNYYWS |
| | CDR-H1 | |
| 936 | Anti-gpNMB | YIYYSGSTYSNPSLKS |
| | CDR-H2 | |
| 937 | Anti-gpNMB | GYNWNYFDY |
| | CDR-H3 | |
| 938 | Anti-gpNMB | RASQSVDNNLV |
| | CDR-L1 | |
| 939 | Anti-gpNMB | GASTRAT |
| | CDR-L2 | |
| 940 | Anti-gpNMB | QQYNNWPPWT |
| | CDR-L3 | |
| 941 | Anti-gpNMB | QVQLQESGPGLVKPSQTLSLTCTVSGGSISSFNYYWSWIRHHPGKGLEWIGYIYYSGSTY |
| | VH | SNPSLKSRVTISVDTSKNQFSLTLSSVTAADTAVYYCARGYNWNYFDYWGQGTLVTVSS |
| 942 | Anti-gpNMB | EIVMTQSPATLSVSPGERATLSCRASQSVDNNLVWYQQKPGQAPRLLIYGASTRATGIPA |
| | VL | RFSGSGSGTEFTLTISSLQSEDFAVYYCQQYNNWPPWTFGQGTKVEIK |
| 943 | Anti-gpNMB | QVQLQESGPGLVKPSQTLSLTCTVSGGSISSFNYYWSWIRHHPGKGLEWIGYIYYSGSTY |
| | HC | SNPSLKSRVTISVDTSKNQFSLTLSSVTAADTAVYYCARGYNWNYFDYWGQGTLVTVSSA |
| | | STKGPSVFPLAPSSKSTSGGTAALGCLVKDYFPEPVTVSWNSGALTSGVHTFPAVLQSSG |
| | | LYSLSSVVTVPSSSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVHNAKTKPREEQYNS |
| | | TYRVVSVLTVLHQDWLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSFFLYSKLTVDKSRWQ |
| | | QGNVFSCSVMHEALHNHYTQKSLSLSPGK |
| 944 | Anti-gpNMB | EIVMTQSPATLSVSPGERATLSCRASQSVDNNLVWYQQKPGQAPRLLIYGASTRATGIPA |
| | LC | RFSGSGSGTEFTLTISSLQSEDFAVYYCQQYNNWPPWTFGQGTKVEIKRTVAAPSVFIFP |
| | | PSDEQLKSGTASVVCLLNNFYPREAKVQWKVDNALQSGNSQESVTEQDSKDSTYSLSSTL |
| | | TLSKADYEKHKVYACEVTHQGLSSPVTKSENRGEC |
| 945 | Anti- | TYWMH |
| | Prolactin | |
| | receptor | |
| | CDR-H1 | |
| 946 | Anti- | EIDPSDSYSNYNQKFKD |
| | Prolactin | |
| | receptor | |
| | CDR-H2 | |
| 947 | Anti- | NGGLGPAWFSY |
| | Prolactin | |
| | receptor | |
| | CDR-H3 | |
| 948 | Anti- | KASQYVGTAVA |
| | Prolactin | |
| | receptor | |
| | CDR-L1 | |
| 949 | Anti- | SASNRYT |
| | Prolactin | |
| | receptor | |
| | CDR-L2 | |
| 950 | Anti- | QQYSSYPWT |
| | Prolactin | |
| | receptor | |
| | CDR-L3 | |
| 951 | Anti- | EVQLVQSGAEVKKPGSSVKVSCKASGYTFTTYWMHWVRQAPGQGLEWIGEIDPSDSYSNY |
| | Prolactin | NQKFKDRATLTVDKSTSTAYMELSSLRSEDTAVYYCARNGGLGPAWFSYWGQGTLVTVSS |
| | receptor VH | |
| 952 | Anti- | DIQMTQSPSSVSASVGDRVTITCKASQYVGTAVAWYQQKPGKSPKLLIYSASNRYTGVPS |
| | Prolactin | RFSDSGSGTDFTLTISSLQPEDFATYFCQQYSSYPWTFGGGTKVEIK |
| | receptor VL | |
| 953 | Anti-FGFR2 | SYAMS |
| | CDR-H1 | |
| 954 | Anti-FGFR2 | AISGSGTSTYYADSVKG |
| | CDR-H2 | |
| 955 | Anti-FGFR2 | VRYNWNHGDWFDP |
| | CDR-H3 | |
| 956 | Anti-FGFR2 | SGSSSNIGNNYVS |
| | CDR-L1 | |
| 957 | Anti-FGFR2 | ENYNRPA |
| | CDR-L2 | |
| 958 | Anti-FGFR2 | SSWDDSLNYWV |
| | CDR-L3 | |
| 959 | Anti-FGFR2 | EVQLLESGGGLVQPGGSLRLSCAASGFTFSSYAMSWVRQAPGKGLEWVSAISGSGTSTYY |
| | VH | ADSVKGRFTISRDNSKNTLYLQMNSLRAEDTAVYYCARVRYNWNHGDWFDPWGQGTLVTV |
| | | SS |
| 960 | Anti-FGFR2 | QSVLTQPPSASGTPGQRVTISCSGSSSNIGNNYVSWYQQLPGTAPKLLIYENYNRPAGVP |
| | VL | DRFSGSKSGTSASLAISGLRSEDEADYYCSSWDDSLNYWVFGGGTKLTVL |
| 961 | Anti-CDCP1 | SYGMS |
| | CDR-H1 | |
| 962 | Anti-CDCP1 | TISSGGSYKYYVDSVKG |
| | CDR-H2 | |
| 963 | Anti-CDCP1 | HPDYDGVWFAY |
| | CDR-H3 | |
| 964 | Anti-CDCP1 | SVSSSVFYVH |
| | CDR-L1 | |
| 965 | Anti-CDCP1 | DTSKLAS |
| | CDR-L2 | |
| 966 | Anti-CDCP1 | QQWNSNPPT |
| | CDR-L3 | |
| 967 | Anti-CDCP1 | EVQLVESGGGLVQPGGSLRLSCAASGFTFNSYGMSWVRQAPGKGLEWVATISSGGSYKYY |
| | VH | VDSVKGRFTISRDNAKNSLYLQMNSLRAEDTAVYYCARHPDYDGVWFAYWGQGTLVTVSS |
| 968 | Anti-CDCP1 | DIQMTQSPSSLSASVGDRVTITCSVSSSVFYVHWYQQKPGKAPKLLIYDTSKLASGVPSR |
| | VL | FSGSGSGTDFTFTISSLQPEDIATYYCQQWNSNPPTFGGGTKVEIK |
| 969 | Anti-CDCP1 | SYGMS |
| | CDR-H1 | |
| 970 | Anti-CDCP1 | TISSGGSYTYYPDSVKG |
| | CDR-H2 | |
| 971 | Anti-CDCP1 | HPDYDGVWFAY |
| | CDR-H3 | |
| 972 | Anti-CDCP1 | SVSSSVFYVH |
| | CDR-L1 | |
| 973 | Anti-CDCP1 | DTSKLAS |
| | CDR-L2 | |
| 974 | Anti-CDCP1 | QQWNSNPPT |
| | CDR-L3 | |
| 975 | Anti-CDCP1 | EVQLVESGGDLVKPGGSLKLSCAASGFTFNSYGMSWVRQTPDKRLEWVATISSGGSYTYY |
| | VH | PDSVKGRFTISRDNAKNTLYLQMSSLKSEDTAMYYCARHPDYDGVWFAYWGQGTLVTVSA |
| 976 | Anti-CDCP1 | QIVLTQSPAIMSASPGEKVTMTCSVSSSVFYVHWYQQKSGTSPKRWIYDTSKLASGVPAR |
| | VL | FSGSGSGTSYSLTISSMEAEDAATYYCQQWNSNPPTFGGGTKLEIK |
| 977 | Anti-CDCP1 | SYYMH |
| | CDR-H1 | |
| 978 | Anti-CDCP1 | IINPSGGSTSYAQKFQG |
| | CDR-H2 | |
| 979 | Anti-CDCP1 | DGVLRYFDWLLDYYYYMDV |
| | CDR-H3 | |
| 980 | Anti-CDCP1 | RASQSVGSYLA |
| | CDR-L1 | |
| 981 | Anti-CDCP1 | DASNRAT |
| | CDR-L2 | |
| 982 | Anti-CDCP1 | QQRANVET |
| | CDR-L3 | |
| 983 | Anti-CDCP1 | EVQLVQSGAEVKKPGASVKVSCKASGYTFTSYYMHWVRQAPGQGLEWMGIINPSGGSTSY |
| | VH | AQKFQGRVTMTRDTSTSTVYMELSSLRSEDTAVYYCARDGVLRYFDWLLDYYYYMDVWGK |
| | | GTTVTVSS |
| 984 | Anti-CDCP1 | EIVLTQSPATLSLSPGERATLSCRASQSVGSYLAWYQQRPGQAPRLLIYDASNRATGIPA |
| | VL | RFSGSGSGTDFTLTISSLEPEDFAVYYCQQRANVFTFGQGTKVEIK |
| 985 | Anti-CDCP1 | SYYMH |
| | CDR-H1 | |
| 986 | Anti-CDCP1 | IINPSGGSTSYAQKFQG |
| | CDR-H2 | |
| 987 | Anti-CDCP1 | DAELRHFDHLLDYHYYMDV |
| | CDR-H3 | |
| 988 | Anti-CDCP1 | RASQSVGSYLA |
| | CDR-L1 | |
| 989 | Anti-CDCP1 | DASNRAT |
| | CDR-L2 | |
| 990 | Anti-CDCP1 | QQRAQEFT |
| | CDR-L3 | |
| 991 | Anti-CDCP1 | EVQLVQSGAEVKKPGASVKVSCKASGYTFTSYYMHWVRQAPGQGLEWMGIINPSGGSTSY |
| | VH | AQKFQGRVTMTRDTSTSTVYMELSSLRSEDTAVYYCARDAELRHFDHLLDYHYYMDVWGQ |
| | | GTTVTVSS |
| 992 | Anti-CDCP1 | EIVMTQSPATLSLSPGERATLSCRASQSVGSYLAWYQQKPGQAPRLLIYDASNRATGIPA |
| | VL | RFSGSGSGTDFTLTISSLQPEDFAVYYCQQRAQEFTFGQGTKVEIK |
| 993 | Anti-ASCT2 | QVQLVQSGSELKKPGAPVKVSCKASGYTFSTFGMSWVRQAPGQGLKWMGWIHTYAGVPIY |
| | VH | GDDFKGRFVFSLDTSVSTAYLQISSLKAEDTAVYFCARRSDNYRYFFDYWGQGTTVTVSS |
| 994 | Anti-ASCT2 | DIQMTQSPSSLSASLGDRVTITCRASQDIRNYLNWYQQKPGKAPKLLIYYTSRLHSGVPS |
| | VL | RFSGSGSGTDYTLTISSLQPEDFATYFCQQGHTLPPTFGQGTKLEIK |
| 995 | Anti-ASCT2 | QIQLVQSGPELKKPGAPVKISCKASGYTFTTFGMSWVKQAPGQGLKWMGWIHTYAGVPIY |
| | VH | GDDFKGRFVFSLDTSVSTAYLQISSVKAEDTATYFCARRSDNYRYFFDYWGQGTTLTVSS |
| 996 | Anti-ASCT2 | DIQMTQSPSSLSASLGDRVTITCRASQDIRNYLNWYQQKPGKAPKLLIYYTSRLHSGVPS |
| | VL | RFSGSGSGTDYTLTISSLQPEDFATYFCQQGHTLPPTFGQGTKLEIK |
| 997 | Anti-ASCT2 | NYYMA |
| | CDR-H1 | |
| 998 | Anti-ASCT2 | SITKGGGNTYYRDSVKG |
| | CDR-H2 | |
| 999 | Anti-ASCT2 | QVTIAAVSTSYFDS |
| | CDR-H3 | |
| 1000 | Anti-ASCT2 | KTNQKVDYYGNSYVY |
| | CDR-L1 | |
| 1001 | Anti-ASCT2 | LASNLAS |
| | CDR-L2 | |
| 1002 | Anti-ASCT2 | QQSRNLPYT |
| | CDR-L3 | |
| 1003 | Anti-ASCT2 | EVQLVESGGGLVQSGRSIRLSCAASGFSFSNYYMAWVRQAPSKGLEWVASITKGGGNTYY |
| | VH | RDSVKGRFTFSRDNAKSTLYLQMDSLRSEDTATYYCARQVTIAAVSTSYFDSWGQGVMVT |
| | | VSS |
| 1004 | Anti-ASCT2 | DIVLTQSPALAVSLGQRATISCKTNQKVDYYGNSYVYWYQQKPGQQPKLLIYLASNLASG |
| | VL | IPARFSGRGSGTDFTLTIDPVEADDTATYYCQQSRNLPYTFGAGTKLELK |
| 1005 | Anti-CD123 | DYYMK |
| | CDR-H1 | |
| 1006 | Anti-CD123 | DIIPSNGATFYNQKFKG |
| | CDR-H2 | |
| 1007 | Anti-CD123 | SHLLRASWFAY |
| | CDR-H3 | |
| 1008 | Anti-CD123 | KSSQSLLNSGNQKNYLT |
| | CDR-L1 | |
| 1009 | Anti-CD123 | WASTRES |
| | CDR-L2 | |
| 1010 | Anti-CD123 | QNDYSYPYT |
| | CDR-L3 | |
| 1011 | Anti-CD123 | QVQLVQSGAEVKKPGASVKMSCKASGYTFTDYYMKWVKQAPGQGLEWIGDIIPSNGATFY |
| | VH | NQKFKGKATLTVDRSISTAYMHLNRLRSDDTAVYYCTRSHLLRASWFAYWGQGTLVTVSS |
| 1012 | Anti-CD123 | DFVMTQSPDSLAVSLGERATINCKSSQSLLNSGNQKNYLTWYLQKPGQPPKLLIYWASTR |
| | VL | ESGVPDRFSGSGSGTDFTLTISSLQAEDVAVYYCQNDYSYPYTFGQGTKLEIK |
| 1013 | Anti-GPC3 | DYEMH |
| | CDR-H1 | |
| 1014 | Anti-GPC3 | GIDPETGGTAYNQKFKG |
| | CDR-H2 | |
| 1015 | Anti-GPC3 | YYSFAY |
| | CDR-H3 | |
| 1016 | Anti-GPC3 | RSSQSIVHSNANTYLQ |
| | CDR-L1 | |
| 1017 | Anti-GPC3 | KVSNRFS |
| | CDR-L2 | |
| 1018 | Anti-GPC3 | FQVSHVPYT |
| | CDR-L3 | |
| 1019 | Anti-GPC3 | EVQLVQSGAEVKKPGATVKISCKVSGYTFTDYEMHWVQQAPGKGLEWMGGIDPETGGTAY |
| | VH | NQKFKGRVTLTADKSTDTAYMELSSLRSEDTAVYYCGRYYSFAYWGQGTLVTVSS |
| 1020 | Anti-GPC3 | DVVMTQSPLSLPVTLGQPASISCRSSQSIVHSNANTYLQWFQQRPGQSPRLLIYKVSNRF |
| | VL | SGVPDRFSGSGSGTDFTLKISRVEAEDVGVYYCFQVSHVPYTFGQGTKLEIK |
| 1021 | Anti-TIGIT | SYAIS |
| | CDR-H1 | |
| 1022 | Anti-TIGIT | SIIPIFGTANYAQKFQG |
| | CDR-H2 | |
| 1023 | Anti-TIGIT | GPSEVGAILGYVWFDP |
| | CDR-H3 | |
| 1024 | Anti-TIGIT | RSSQSLLHSNGYNYLD |
| | CDR-L1 | |
| 1025 | Anti-TIGIT | LGSNRAS |
| | CDR-L2 | |
| 1026 | Anti-TIGIT | MQARRIPIT |
| | CDR-L3 | |
| 1027 | Anti-TIGIT | QVQLVQSGAEVKKPGSSVKVSCKASGGTFSSYAISWVRQAPGQGLEWMGSIIPIFGTANY |
| | VH | AQKFQGRVTITADESTSTAYMELSSLRSEDTAVYYCARGPSEVGAILGYVWEDPWGQGTL |
| | | VTVSS |
| 1028 | Anti-TIGIT | DIVMTQSPLSLPVTPGEPASISCRSSQSLLHSNGYNYLDWYLQKPGQSPQLLIYLGSNRA |
| | VL | SGVPDRFSGSGSGTDFTLKISRVEAEDVGVYYCMQARRIPITFGGGTKVEIK |
| 1029 | Anti-CD33 | NYDIN |
| | CDR-H1 | |
| 1030 | Anti-CD33 | WIYPGDGSTKYNEKFKA |
| | CDR-H2 | |
| 1031 | Anti-CD33 | GYEDAMDY |
| | CDR-H3 | |
| 1032 | Anti-CD33 | KASQDINSYLS |
| | CDR-L1 | |
| 1033 | Anti-CD33 | RANRLVD |
| | CDR-L2 | |
| 1034 | Anti-CD33 | LQYDEFPLT |
| | CDR-L3 | |
| 1035 | Anti-CD33 | QVQLVQSGAE VKKPGASVKV SCKASGYTFT NYDINWVRQA PGQGLEWIGW |
| | VH | IYPGDGSTKY NEKFKAKATL TADTSTSTAY MELRSLRSDD TAVYYCASGY |
| | | EDAMDYWGQG TTVTVSS |
| 1036 | Anti-CD33 | DIQMTQSPSSLSASVGDRVTINCKASQDINSYLSWFQQKPGKAPKTLIYRANRVDGVPSR |
| | VL | FSGSGSGQDYTLTISSLQPEDFATYYCLQYDEFPLTFGGGTKVEIK |
| 1037 | Anti-BCMA | DYYIH |
| | CDR-H1 | |
| 1038 | Anti-BCMA | YINPNSGYTNYAQKFQG |
| | CDR-H2 | |
| 1039 | Anti-BCMA | YMWERVTGFFDE |
| | CDR-H3 | |
| 1040 | Anti-BCMA | LASEDISDDLA |
| | CDR-L1 | |
| 1041 | Anti-BCMA | TTSSLQS |
| | CDR-L2 | |
| 1042 | Anti-BCMA | QQTYKFPPT |
| | CDR-L3 | |
| 1043 | Anti-BCMA | QVQLVQSGAEVKKPGASVKLSCKASGYTFTDYYIHWVRQAPGQGLEWIGYINPNSGYTNY |
| | VH | AQKFQGRATMTADKSINTAYVELSRLRSDDTAVYFCTRYMWERVTGFFDFWGQGTMVTVS |
| | | S |
| 1044 | Anti-BCMA | DIQMTQSPSSVSASVGDRVTITCLASEDISDDLAWYQQKPGKAPKVLVYTTSSLQSGVPS |
| | VL | RFSGSGSGTDFTLTISSLQPEDFATYFCQQTYKFPPTFGGGTKVEIK |

What is claimed is:

1. A compound of Formula (I): 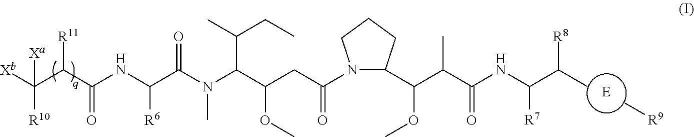
or a salt thereof, wherein
X^{b} is —NR¹R²; and X^{a} is 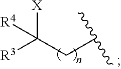
or
X^{a} and X^{b} are taken together with the carbon atom to which they are attached to form 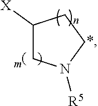
wherein the asterisk denotes the carbon atom of Formula (I) that bears the X^{a} and X^{b} groups;
R¹, R², R³, R⁴, R^{a}, R^{b}, R⁵, and R¹⁰ are each independently H or C₁-C₄ alkyl;
X is H, OH, —C(O)NR^{a}R^{b}, —S(O)₂R^{a}, —S(O)—R^{a} —S(O)₂NR^{a}R^{b}, —NHS(O)₂R^{a}, or —NHC(O)R^{a};
R⁶ is C₁-C₄ alkyl optionally substituted with OH;
R⁷ is H, C₁-C₄ alkyl optionally substituted with one or two OH moieties, or 5-6 membered heteroaryl;
E is phenyl or 5-6 membered heteroaryl;
R⁸, R⁹, and R¹¹ are each independently H or OH;
n is 0, 1, 2, or 3;
m is 1, 2, 3, or 4; and
q is 0 or 1,
wherein when X is H, at least two of R⁷, R⁸, R⁹, and R¹¹ comprise an OH moiety.

2. A compound of Formula (II): 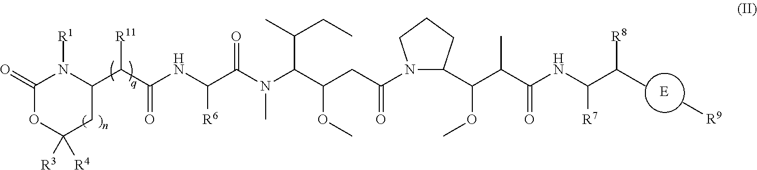
or a salt thereof, wherein
R¹, R³, and R⁴ are independently H or C₁-C₄ alkyl;
R⁶ is C₁-C₄ alkyl optionally substituted with OH;
R⁷ is H, C₁-C₄ alkyl optionally substituted with one or two OH moieties, or 5-6 membered heteroaryl;
E is phenyl or 5-6 membered heteroaryl;
R⁸, R⁹, and R¹¹ are each independently H or OH;
n is 0, 1, or 2; and
q is 0 or 1.

3. The compound of claim 1 or 2, wherein q is 0.

4. The compound of claim 1 or 2, wherein q is 1.

5. The compound of claim 1, or a salt thereof, wherein
X is OH.

6. The compound of claim 1, or a salt thereof, wherein
X is —C(O)NR^{a}R^{b}, —S(O)₂R^{a}, —S(O)—R^{a} —S(O)₂NR^{a}R^{b}, —NHS(O)₂R^{a}, or —NHC(O)R^{a}.

7. The compound of any one of claims 1 or 3-6, or a salt thereof, wherein
X^{b} is —NR¹R²; and
X^{a} is

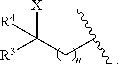

8. The compound of any one of claims 1-7, or a salt thereof, wherein R³ is H.

9. The compound of any one of claims 1-8, or a salt thereof, wherein R⁴ is H.

10. The compound of any one of claims 1-9, or a salt thereof, wherein n is 0 or 1.

11. The compound of any one of claims 1 or 3-6, or a salt thereof, wherein
X^{a} and X^{b} are taken together with the carbon atom to which they are attached to form 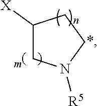
wherein the asterisk denotes the carbon atom of Formula (I) that bears the X^{a} and X^{b} groups.

12. The compound of any one of claims 1 or 3-11, wherein m is 1 or 2.

13. The compound of any one of claims 1-10, or a salt thereof, wherein R¹ is H.

14. The compound of any one of claims 1-10, or a salt thereof, wherein R¹ is C₁-C₄ alkyl.

15. The compound of any one of claims 1-10 or 14, or a salt thereof, wherein R¹ is methyl.

16. The compound of any one of claims 1, 3-10, or 13-15, or a salt thereof, wherein R² is methyl.

17. The compound of any one of claims 1-16, or a salt thereof, wherein R¹⁰ is H.

18. The compound of any one of claims 1-16, or a salt thereof, wherein R¹⁰ is methyl.

19. The compound of any one of claims 1-18, or a salt thereof, wherein R⁶ is unsubstituted C₁-C₄ alkyl.

20. The compound of any one of claims 1-19, or a salt thereof, wherein R⁶ is isopropyl.

21. The compound of any one of claims 1-18, or a salt thereof, wherein R⁶ is C₁-C₄ alkyl substituted with OH.

22. The compound of any one of claims 1-21, or a salt thereof, wherein R⁷ C₁-C₄ alkyl substituted with OH.

23. The compound of any one of claims 1-22, or a salt thereof, wherein R⁷ is —CH₂OH.

24. The compound of any one of claims 1-21, or a salt thereof, wherein R⁷ is H.

25. The compound of any one of claims 1-21, or a salt thereof, wherein R⁷ is unsubstituted C₁-C₄ alkyl.

26. The compound of any one of claims 1-21 or 25, or a salt thereof, wherein R⁷ is methyl.

27. The compound of any one of claims 1-21, or a salt thereof, wherein R⁷ is 5-6 membered heteroaryl.

28. The compound of any one of claims 1-27, or a salt thereof, wherein R⁸ is H.

29. The compound of any one of claims 1-28, or a salt thereof, wherein R⁸ is OH.

30. The compound of any one of claims 1-29, or a salt thereof, wherein E is phenyl.

31. The compound of any one of claims 1-30, or a salt thereof, wherein E-R⁹ is 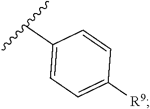
and
the wavy line indicates the point of attachment of E to the rest of the compound.

32. The compound of any one of claims 1-29, or a salt thereof, wherein E is 5-6 membered heteroaryl.

33. The compound of any one of claims 1-32, or a salt thereof, wherein R⁹ is H.

34. The compound of any one of claims 1-33, or a salt thereof, wherein R⁹ is OH.

35. The compound of claim 1, or a salt thereof, wherein
X^{a} is 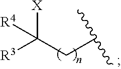
X^{b} is —NR¹R²;
R¹ is H or methyl;
R² is methyl;
X is OH;
R³ and R⁴ are H;
R⁶ is isopropyl;
R⁷ is —CH₂OH;
R⁸ is H;
E is phenyl; and
R⁹ is H.

36. The compound of claim 1, wherein the compound is 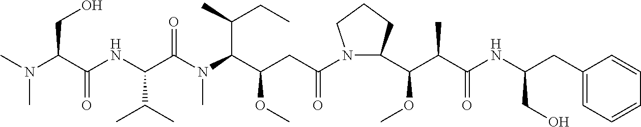 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H](CO)N(C)C)C(C)C]
or a salt thereof.

37. A Drug-Linker compound of the following formula:
Q-D,
or a salt thereof, wherein
Q is a Linker Unit selected from the group consisting of:
(i) Z′-A-RL-,
(ii) Z′-A-RL-Y—,
(iii) Z′-A-S*—RL-,
(iv) Z′-A-S*—RL-Y—,
(v) Z′-A-B(S*)—RL-,
(vi) Z′-A-B(S*)—RL-Y—,
(vii) Z′-A-,
(viii) Z′-A-S*—W—,
(ix) Z′-A-B(S*)—W—,
(x) Z′-A-S*—W-RL-, and
(xi) Z′-A-B(S*)—W-RL-;
Z′ is a Stretcher Unit precursor;
A is a bond or a Connector Unit;
B is a Parallel Connector Unit;
S* is a Partitioning Agent;
RL is a Releasable Linker;
W is an Amino Acid Unit;
Y is a Spacer Unit; and
D is a Drug Unit of Formula (I′): 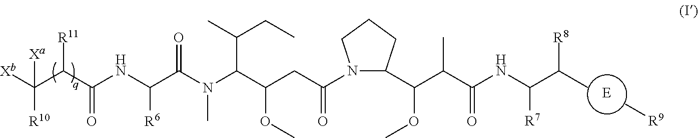
wherein
X^{b} is —NR²—^{#} or —N⁺R¹R⁵—^{#}, wherein the # denotes the point of attachment to Q, and X^{a} is 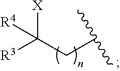
or
X^{a} and X^{b} are taken together with the carbon atom to which they are attached to form 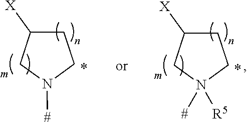
wherein the asterisk denotes the carbon atom of Formula (I) that bears the X^{a} and X^{b} groups, and the # denotes the point of attachment to Q;
R¹ and R⁵ are independently C₁-C₄ alkyl;
X is H, OH, —C(O)NR^{a}R^{b}, —S(O)₂R^{a}, —S(O)—R^{a} —S(O)₂NR^{a}R^{b}, —NHS(O)₂R^{a}, or —NHC(O)R^{a};
R², R³, R⁴, R¹⁰, R^{a}, and R^{b} are each independently H or C₁-C₄ alkyl;
R⁶ is C₁-C₄ alkyl optionally substituted with OH;
R⁷ is H, C₁-C₄ alkyl optionally substituted with one or two OH moieties, or 5-6 membered heteroaryl;
E is phenyl or 5-6 membered heteroaryl;
R⁸, R⁹, and R¹¹ are each independently H or OH;
n is 0, 1, 2, or 3;
m is 1, 2, 3, or 4;
q is 0 or 1; and
wherein when X is H, at least two of R⁷, R⁸, R⁹, and R¹¹ comprise an OH moiety.

38. The Drug-Linker compound of claim 37, or a salt thereof, wherein the Linker Unit Q is of formula (i), (ii), (iii), (iv), (x), or (xi).

39. The Drug-Linker compound of claim 37, or a salt thereof, wherein the Linker Unit Q is of formula (v), (vi), (ix), or (xi).

40. The Drug-Linker compound of claim 37, or a salt thereof, wherein the Linker Unit Q is of formula (viii), (ix), (x), or (xi).

41. The Drug-Linker compound of any one of claims 37-40, or a salt thereof, wherein the Stretcher Unit Z′ is 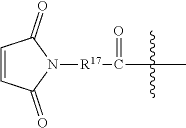
wherein
R¹⁷ is —CH₂CH₂(OCH₂CH₂)ₖ—, —C₁-C₁₀ alkylene-, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—;
subscript k is an integer ranging from 1 to 36;
R¹⁷ is optionally substituted by a Basic Unit (BU) such as an aminoalkyl moiety, e.g. —(CH₂)ₓNH₂, —(CH₂)ₓNHR^{a}, and —(CH₂)ₓNR^{a}₂, wherein x is an integer of from 1-4 and each R^{a} is independently selected from the group consisting of C₁₋₆ alkyl and C₁₋₆ haloalkyl, or two R^{a} groups are combined with the nitrogen to which they are attached to form an azetidinyl, pyrrolidinyl or piperidinyl group; and
the wavy line indicates the point of covalent attachment to the rest of the Drug-Linker compound.

42. The Drug-Linker of any one of claims 37-41, or a salt thereof, wherein the Stretcher Unit Z′ is 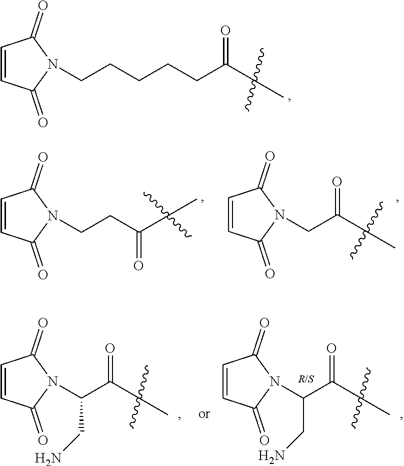
wherein the wavy lines indicate the point of covalent attachment to the rest of the Drug-Linker compound.

43. The Drug-Linker compound of any one of claims 37-42, or a salt thereof, wherein the Connector Unit A is 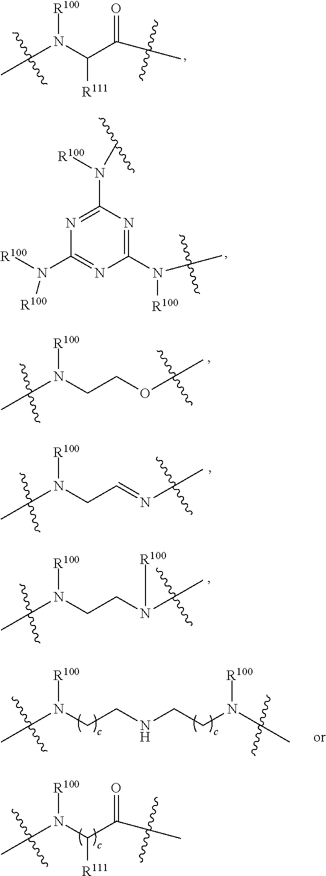
wherein
each R¹⁰⁰ is independently selected from hydrogen or —C₁-C₃ alkyl;
R¹¹¹ is independently selected from the group consisting of hydrogen, p-hydroxybenzyl, methyl, isopropyl, isobutyl, sec-butyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, 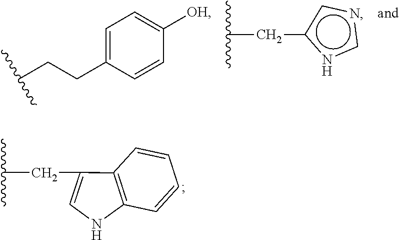
each subscript c is an independently selected integer from 1 to 10; and
the wavy lines indicate attachment of the Connector Unit to the rest of the Drug-Linker compound.

44. The Drug-Linker compound of any one of claims 37-43, or a salt thereof, wherein the Connector Unit A is 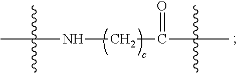
c is an integer ranging from 1 to 6; and
the wavy lines indicate the site of attachment to the rest of the Drug-Linker compound or salt thereof.

45. The Drug-Linker compound of any one of claims 37-42, or a salt thereof, wherein A is a bond.

46. The Drug-Linker compound of claims 37-45, or a salt thereof, wherein B is 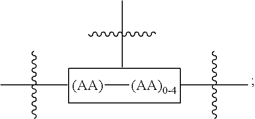
each AA is independently a proteinogenic or non-proteinogenic amino acid; and
the wavy lines indicate points of attachment to the rest of the Drug-Linker compound or salt thereof.

47. The Drug-Linker compound of any one of claims 37-46, or a salt thereof, wherein B is an amino acid.

48. The Drug-Linker compound of any one of claims 37-47, or a salt thereof, wherein B is 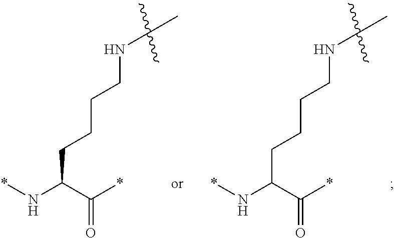 [SMILES: *NCCCC[C@H](N*)C(*)=O ; *NCCCCC(N*)C(*)=O]
the wavy line indicates the point of attachment to the Partitioning Agent S*; and
the asterisks indicate points of attachment to the rest of the Drug-Linker structure.

49. The Drug-Linker compound of any one of claims 37-48, or a salt thereof, wherein the Partitioning Agent S* is a polyethylene glycol (PEG) unit, cyclodextrin unit, polyamide, hydrophilic peptide, polysaccharide, or dendrimer.

50. The Drug-Linker compound of any one of claims 37-49, or a salt thereof, wherein the Partitioning Agent S* is a PEG Unit comprising from 4 to 72 (CH₂CH₂O) subunits.

51. The Drug Linker of any one of claims 37-50, or a salt thereof, wherein the PEG Unit is 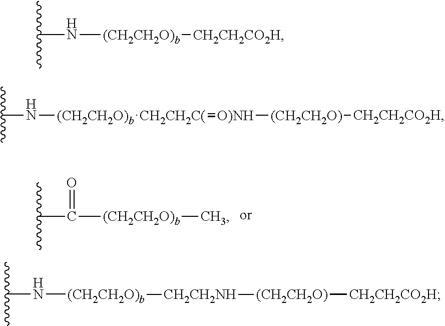
b is selected from the group consisting of 4 to 36; and
the wavy lines indicate the site of attachment to the rest of the Drug-Linker compound or salt thereof.

52. The Drug-Linker compound of any one of claims 37-51, or a salt thereof, wherein the Releasable Linker RL is
-(AA)₁₋₁₂-; and
each AA is independently a proteinogenic or non-proteinogenic amino acid.

53. The Drug-Linker compound of any one of claims 37-52, or a salt thereof, wherein the Releasable Linker RL is
-AA₁-AA₂- or -AA₁-AA₂-AA₃-, wherein AA₁ is attached to the Stretcher Unit Z′ or the Connector Unit A.

54. The Drug-Linker compound of any one of claims 37-53, or a salt thereof, wherein the Releasable Linker RL is 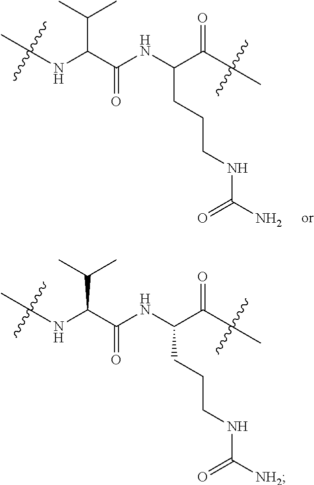 [SMILES: *NC(C(=O)NC(CCCNC(N)=O)C(*)=O)C(C)C ; *N[C@H](C(=O)N[C@@H](CCCNC(N)=O)C(*)=O)C(C)C]
and
the wavy line adjacent to the —NH— group indicates attachment to the Stretcher Unit Z′ or the Connector Unit A and the wavy line adjacent to the —C(═O)— group indicates attachment to the Spacer Unit Y or the Drug Unit D.

55. The Drug-Linker compound of any one of claims 37-54, or a salt thereof, wherein the Releasable Linker RL is a glycoside.

56. The Drug-Linker compound of any one of claims 37-55, or a salt thereof, wherein the Releasable Linker RL is 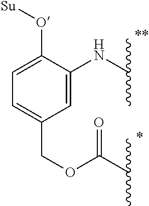
wherein
Su is a hexose form a monosaccharide;
O′ represents the oxygen atom of a glycosidic bond that is capable of cleavage by a glycosidase;
the wavy line marked with a single asterisk (*) indicates the site of covalent attachment to D; and
the wavy line marked with a double asterisk (**) indicates the site of covalent attachment to the remainder of Q.

57. The Drug-Linker compound of any one of claims 37-56, or a salt thereof, wherein the Releasable Linker RL is 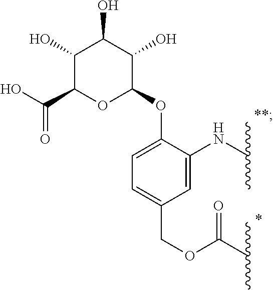 [SMILES: *Nc1cc(COC(*)=O)ccc1O[C@H]1O[C@@H](C(=O)O)[C@H](O)[C@@H](O)[C@@H]1O]
the wavy line marked with a single asterisk (*) indicates the site of covalent attachment to D; and
the wavy line marked with a double asterisk (**) indicates the site of covalent attachment to the remainder of Q.

58. The Drug-Linker compound of any one of claims 37-39 or 41-57, or a salt thereof, wherein the Spacer Unit Y is 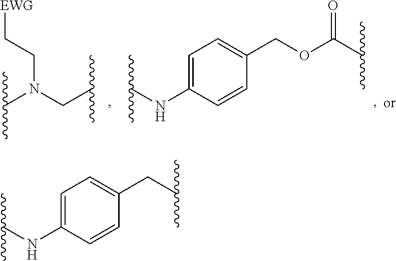
wherein EWG is an electron-withdrawing group; and
the wavy lines indicate the site of attachment to the rest of the Drug-Linker compound or salt thereof.

59. The Drug-Linker compound of any one of claims 37-39 or 41-58, or a salt thereof, wherein the Spacer Unit Y is 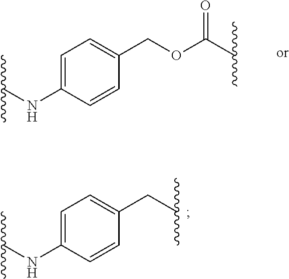 [SMILES: *Nc1ccc(COC(*)=O)cc1 ; *Cc1ccc(N*)cc1]
and
the wavy lines indicate the site of attachment to the rest of the Drug-Linker compound or salt thereof.

60. The Drug-Linker compound of any one of claims 37, 41-42, 45-48, 52-54, or 58-59, or a salt thereof, wherein
Z′ is 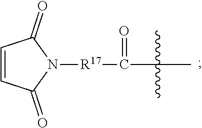
R¹⁷ is C₁-C₁₀ alkylene;
A is a bond;
RL is -AA₁-AA₂-;
AA₁ and AA₂ are each independently a proteinogenic amino acid;
Y is 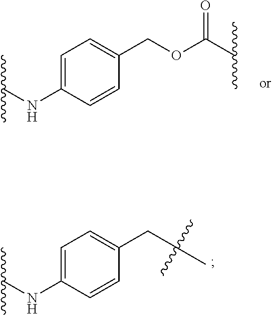 [SMILES: *Nc1ccc(COC(*)=O)cc1 ; *Cc1ccc(N*)cc1]
and
the wavy lines indicate the site of attachment to the rest of the Drug-Linker compound or salt thereof.

61. The Drug-Linker compound of any one of claims 37, 41-42, 45-48, 52-54, or 58-60, or a salt thereof, wherein
Z′ is 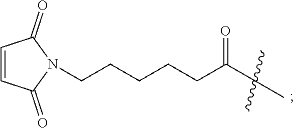 [SMILES: *C(=O)CCCCCN1C(=O)C=CC1=O]
A is a bond;
RL is 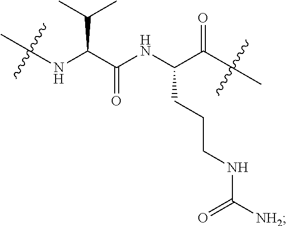 [SMILES: *N[C@H](C(=O)N[C@@H](CCCNC(N)=O)C(*)=O)C(C)C]
Y is

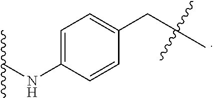 [SMILES: *Cc1ccc(N*)cc1]

62. The Drug-Linker compound of any one of claims 37-38 or 41-61, wherein Y-D is 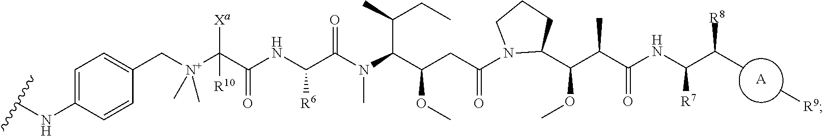
and
the wavy line indicates the site of attachment to the rest of the Drug-Linker compound or salt thereof.

63. The Drug-Linker compound of any one of claims 37-38 or 41-61, wherein Y-D is 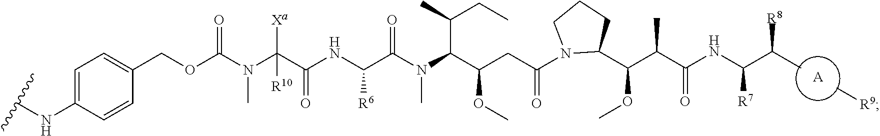
and
the wavy line indicates the site of attachment to the rest of the Drug-Linker compound or salt thereof.

64. The Drug-Linker compound of any one of claims 37, 41-42, 45-48, 52-54, 58-61, or 62, wherein the compound is 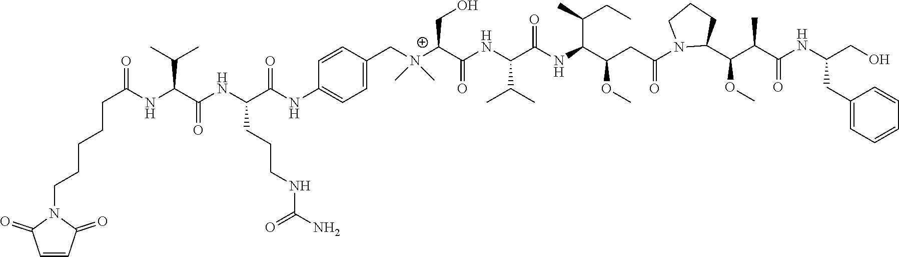 [SMILES: CC[C@H](C)[C@H](NC(=O)[C@@H](NC(=O)[C@H](CO)[N+](C)(C)Cc1ccc(NC(=O)[C@H](CCCNC(N)=O)NC(=O)[C@@H](NC(=O)CCCCCN2C(=O)C=CC2=O)C(C)C)cc1)C(C)C)[C@@H](CC(=O)N1CCC[C@H]1[C@H](OC)[C@@H](C)C(=O)N[C@H](CO)Cc1ccccc1)OC]
or a salt thereof.

65. The Drug-Linker compound of any one of claims 37, 41-42, 45-48, 52-54, 58-61, or 63, wherein the compound is 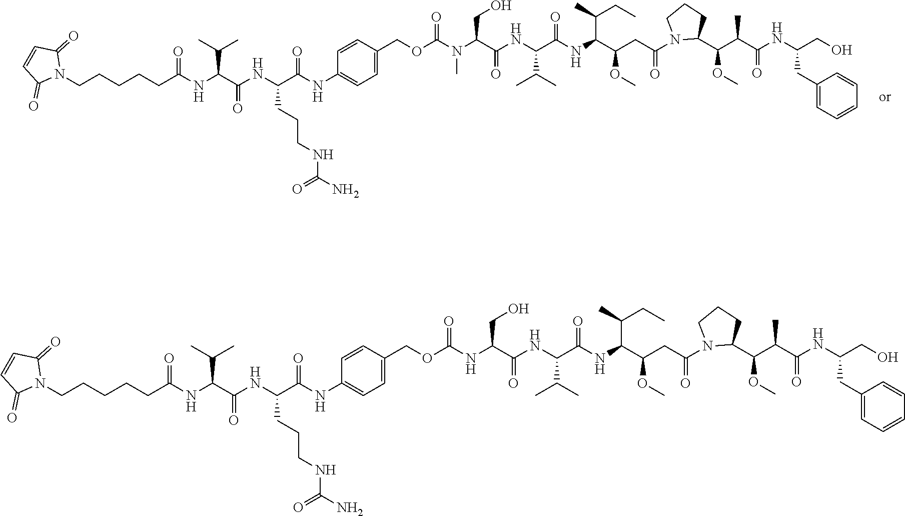 [SMILES: CC[C@H](C)[C@H](NC(=O)[C@@H](NC(=O)[C@H](CO)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C)[C@@H](CC(=O)N1CCC[C@H]1[C@H](OC)[C@@H](C)C(=O)N[C@H](CO)Cc1ccccc1)OC ; CC[C@H](C)[C@H](NC(=O)[C@@H](NC(=O)[C@H](CO)NC(=O)OCc1ccc(NC(=O)[C@H](CCCNC(N)=O)NC(=O)[C@@H](NC(=O)CCCCCN2C(=O)C=CC2=O)C(C)C)cc1)C(C)C)[C@@H](CC(=O)N1CCC[C@H]1[C@H](OC)[C@@H](C)C(=O)N[C@H](CO)Cc1ccccc1)OC]
or a salt thereof.

66. A Ligand-Drug Conjugate compound of the formula:
L-(Q-D)ₚ
or a pharmaceutically acceptable salt thereof, wherein
L is a Ligand Unit;
Q is a Linker Unit selected from the group consisting of:
(i) Z′-A-RL-,
(ii) Z′-A-RL-Y—,
(iii) Z′-A-S*—RL-,
(iv) Z′-A-S*—RL-Y—,
(v) Z′-A-B(S*)—RL-,
(vi) Z′-A-B(S*)—RL-Y—,
(vii) Z′-A-,
(viii) Z′-A-S*—W—,
(ix) Z′-A-B(S*)—W—,
(x) Z′-A-S*—W-RL-, and
(xi) Z′-A-B(S*)—W-RL-;
Z′ is a Stretcher Unit;
A is a bond or a Connector Unit;
B is a Parallel Connector Unit;
S* is a Partitioning Agent;
RL is a Releasable Linker;
W is a Amino Acid Unit;
Y is a Spacer Unit; and
D is a Drug Unit of Formula (I′): 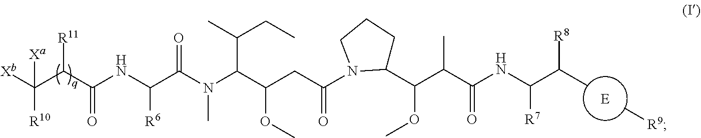
wherein
X^{b} is —NR²—^{#} or —N⁺R¹R⁵—^{#}, wherein the # denotes the point of attachment to Q, and X^{a} is 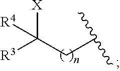
or
X^{a} and X^{b} are taken together with the carbon atom to which they are attached to form 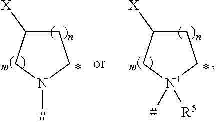
wherein the asterisk denotes the carbon atom of Formula (I) that bears the X^{a} and X^{b} groups, and the # denotes the point of attachment to Q;
R¹ and R⁵ are independently C₁-C₄ alkyl;
X is H, OH, —C(O)NR^{a}R^{b}, —S(O)₂R^{a}, —S(O)—R^{a} —S(O)₂NR^{a}R^{b}, —NHS(O)₂R^{a}, or —NHC(O)R^{a};
R², R³, R⁴, R¹⁰, R^{a}, and R^{b} are each independently H or C₁-C₄ alkyl;
R⁶ is C₁-C₄ alkyl optionally substituted with OH;
R⁷ is H, C₁-C₄ alkyl optionally substituted with one or two OH moieties, or 5-6 membered heteroaryl;
E is phenyl or 5-6 membered heteroaryl;
R⁸, R⁹, and R¹¹ are each independently H or OH;
n is 0, 1, 2, or 3;
m is 1, 2, 3, or 4;
q is 0 or 1;
p is an integer ranging from 1 to 12; and
wherein when X is H, at least two of R⁷, R⁸, R⁹, and R¹¹ comprise an OH moiety.

67. The Ligand-Drug Conjugate compound of claim 66, or a pharmaceutically acceptable salt thereof, wherein the Linker Unit Q is of formula (i), (ii), (iii), (iv), (x), or (xi).

68. The Ligand-Drug Conjugate compound of claim 66, or a pharmaceutically acceptable salt thereof, wherein the Linker Unit Q is of formula (v), (vi), (ix), or (xi).

69. The Ligand-Drug Conjugate compound of claim 66, or a pharmaceutically acceptable salt thereof, wherein the Linker Unit Q is of formula (viii), (ix), (x), or (xi).

70. The Ligand-Drug Conjugate compound of any one of claims 66-69, or a pharmaceutically acceptable salt thereof, wherein the Ligand Unit L and the Stretcher Unit Z together are 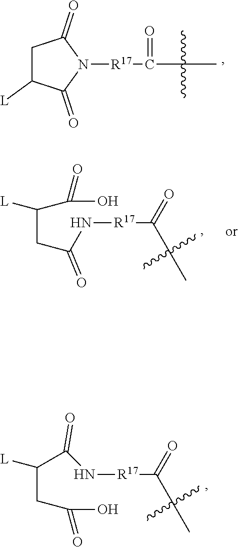
wherein
R¹⁷ is —CH₂CH₂(OCH₂CH₂)ₖ—, —C₁-C₁₀ alkylene-, C₁-C₁₀ heteroalkylene-, —C₃-C₈ carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₃-C₈ carbocyclo-C(═O)—, —O—(C₁-C₈ alkylene)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, C₁-C₁₀ heteroalkylene-NH—, —C₃-C₈ carbocyclo-NH—, —O—(C₁-C₈ alkylene)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkylene)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—;
subscript k is an integer ranging from 1 to 36;
R¹⁷ is optionally substituted by a Basic Unit (BU) such as an aminoalkyl moiety, e.g. —(CH₂)ₓNH₂, —(CH₂)ₓNHR^{a}, and —(CH₂)ₓNR^{a}₂, wherein x is an integer of from 1-4 and each R^{a} is independently selected from the group consisting of C₁₋₆ alkyl and C₁₋₆ haloalkyl, or two R^{a} groups are combined with the nitrogen to which they are attached to form an azetidinyl, pyrrolidinyl or piperidinyl group; and
the wavy line indicates the point of covalent attachment to the rest of the Ligand-Drug Conjugate compound.

71. The Ligand-Drug Conjugate compound of any one of claims 66-70, or a pharmaceutically acceptable salt thereof, wherein the Ligand Unit L and the Stretcher Unit Z together are 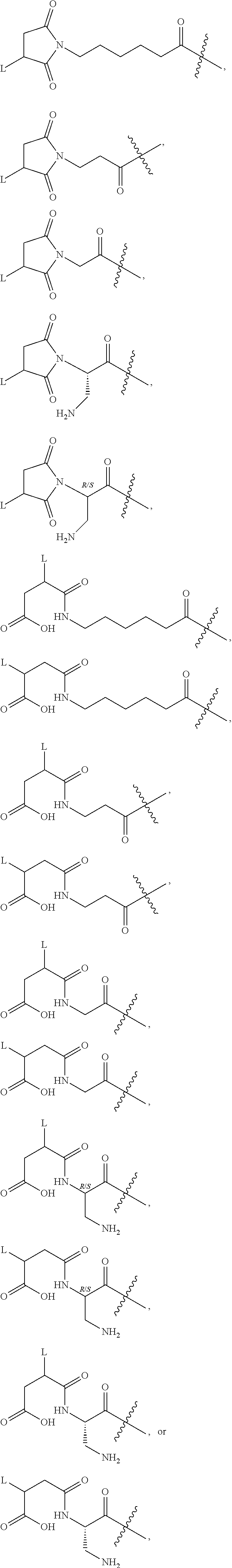
wherein the wavy lines indicate the point of covalent attachment to the rest of the Ligand-Drug Conjugate compound.

72. The Ligand-Drug Conjugate compound of any one of claims 66-71, or a pharmaceutically acceptable salt thereof, wherein the Connector Unit A is 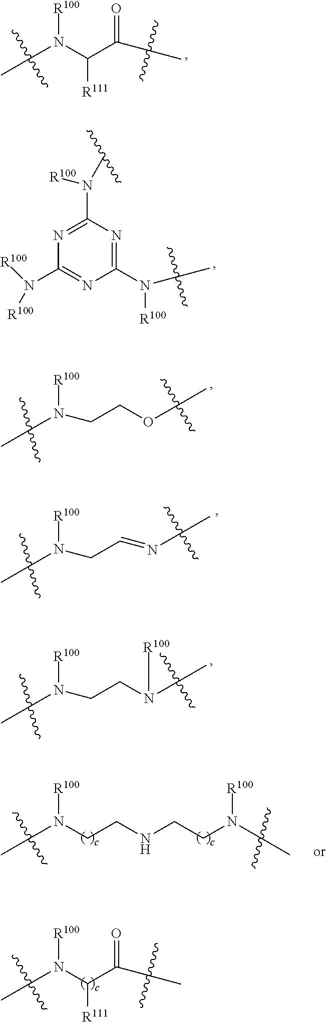
wherein
each R¹⁰⁰ is independently selected from hydrogen or —C₁-C₃ alkyl;
R¹¹¹ is independently selected from the group consisting of hydrogen, p-hydroxybenzyl, methyl, isopropyl, isobutyl, sec-butyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, 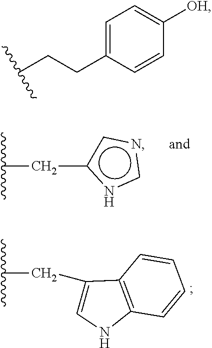
each subscript c is an independently selected integer from 1 to 10; and
the wavy lines indicate attachment of the Connector Unit to the rest of the Ligand-Drug Conjugate compound or pharmaceutically acceptable salt thereof.

73. The Ligand-Drug Conjugate compound of any one of claims 66-72, or a pharmaceutically acceptable salt thereof, wherein the Connector Unit A is 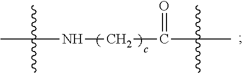
c is an integer ranging from 1 to 6; and
the wavy lines indicate the site of attachment to the rest of the Ligand-Drug Conjugate compound or pharmaceutically acceptable salt thereof.

74. The Ligand-Drug Conjugate compound of any one of claims 66-71, or a pharmaceutically acceptable salt thereof, wherein A is a bond.

75. The Ligand-Drug Conjugate compound of claims 66-74, or a salt thereof, wherein B is 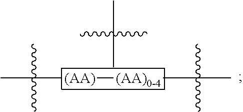
each AA is independently a proteinogenic or non-proteinogenic amino acid; and
the wavy lines indicate points of attachment to the rest of the Ligand-Drug Conjugate compound or pharmaceutically acceptable salt thereof.

76. The Ligand-Drug Conjugate compound of any one of claims 66-75, or a pharmaceutically acceptable salt thereof, wherein B is an amino acid.

77. The Ligand-Drug Conjugate compound of any one of claims 66-76, or a pharmaceutically acceptable salt thereof, wherein B is 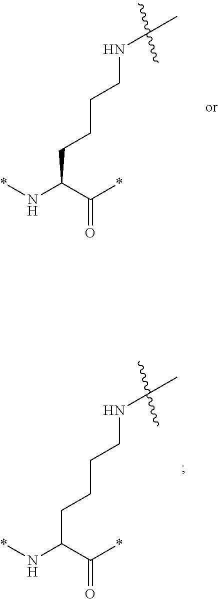 [SMILES: *NCCCC[C@H](N*)C(*)=O ; *NCCCCC(N*)C(*)=O]
the wavy line indicates the point of attachment to the Partitioning Agent S*; and
the asterisks indicate points of attachment to the rest of the Ligand-Drug Conjugate compound or pharmaceutically acceptable salt thereof.

78. The Ligand-Drug Conjugate compound of any one of claims 66-77, or a pharmaceutically acceptable salt thereof, wherein the Partitioning Agent S* is a polyethylene glycol (PEG) unit, cyclodextrin unit, polyamide, hydrophilic peptide, polysaccharide, or dendrimer.

79. The Ligand-Drug Conjugate compound of any one of claims 66-78, or a pharmaceutically acceptable salt thereof, wherein the Partitioning Agent S* is a PEG Unit comprising from 4 to 72 (CH₂CH₂O) subunits.

80. The Ligand-Drug Conjugate compound of any one of claims 66-79, or a pharmaceutically acceptable salt thereof, wherein the PEG Unit is 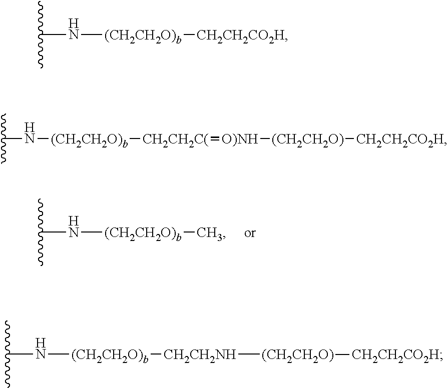
b is selected from the group consisting of 4 to 36; and
the wavy lines indicate the site of attachment to the rest of the Ligand-Drug Conjugate compound or pharmaceutically acceptable salt thereof.

81. The Ligand-Drug Conjugate compound of any one of claims 66-80, or a pharmaceutically acceptable salt thereof, wherein the Releasable Linker RL is
-(AA)₁₋₁₂-; and
each AA is independently a proteinogenic or non-proteinogenic amino acid.

82. The Ligand-Drug Conjugate compound of any one of claims 66-81, or a pharmaceutically acceptable salt thereof, wherein the Releasable Linker RL is
-AA₁-AA₂- or -AA₁-AA₂-AA₃-, wherein AA₁ is attached to the Stretcher Unit Z or the Connector Unit A.

83. The Ligand-Drug Conjugate compound of any one of claims 66-82, or a pharmaceutically acceptable salt thereof, wherein the Releasable Linker RL is 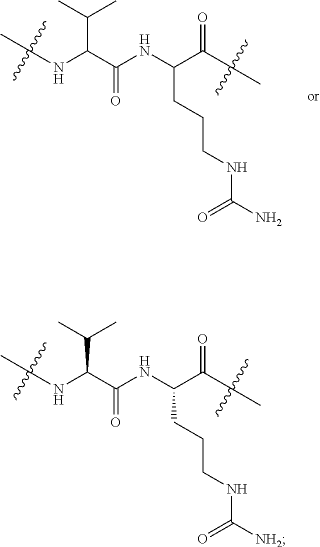 [SMILES: *NC(C(=O)NC(CCCNC(N)=O)C(*)=O)C(C)C ; *N[C@H](C(=O)N[C@@H](CCCNC(N)=O)C(*)=O)C(C)C]
and
the wavy line adjacent to the —NH— group indicates attachment to the Stretcher Unit Z or the Connector Unit A and the wavy line adjacent to the —C(═O)— group indicates attachment to the Spacer Unit Y or the Drug Unit D.

84. The Ligand-Drug Conjugate compound of any one of claims 66-83, or a pharmaceutically acceptable salt thereof, wherein the Releasable Linker RL is a glycoside.

85. The Ligand-Drug Conjugate compound of any one of claims 66-84, or a pharmaceutically acceptable salt thereof, wherein the Releasable Linker RL is 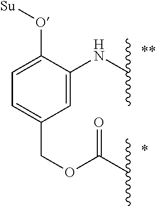
wherein
Su is a hexose form a monosaccharide;
O′ represents the oxygen atom of a glycosidic bond that is capable of cleavage by a glycosidase;
the wavy line marked with a single asterisk (*) indicates the site of covalent attachment to D; and
the wavy line marked with a double asterisk (**) indicates the site of covalent attachment to the remainder of Q.

86. The Ligand-Drug Conjugate compound of any one of claims 66-85, or a pharmaceutically acceptable salt thereof, wherein the Releasable Linker RL is 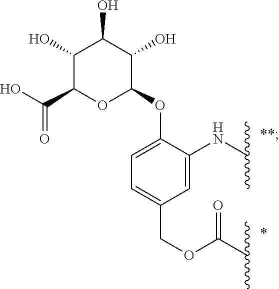 [SMILES: *Nc1cc(COC(*)=O)ccc1O[C@H]1O[C@@H](C(=O)O)[C@H](O)[C@@H](O)[C@@H]1O]
the wavy line marked with a single asterisk (*) indicates the site of covalent attachment to D; and
the wavy line marked with a double asterisk (**) indicates the site of covalent attachment to the remainder of Q.

87. The Ligand-Drug Conjugate compound of any one of claims 66-68 or 70-86, or a pharmaceutically acceptable salt thereof, wherein the Spacer Unit Y is 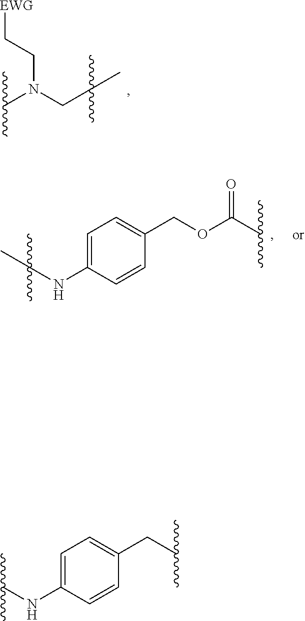
wherein EWG is an electron-withdrawing group; and
the wavy lines indicate the site of attachment to the rest of the Ligand-Drug Conjugate compound or pharmaceutically acceptable salt thereof.

88. The Ligand-Drug Conjugate compound of any one of claims 66-68 or 70-87, or a pharmaceutically acceptable salt thereof, wherein the Spacer Unit Y is 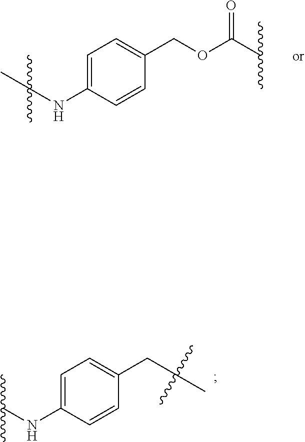 [SMILES: *Nc1ccc(COC(*)=O)cc1 ; *Cc1ccc(N*)cc1]
and
the wavy lines indicate the site of attachment to the rest of the Ligand-Drug Conjugate compound or pharmaceutically acceptable salt thereof.

89. The Ligand-Drug Conjugate compound of any one of claims 66-67, 70-71, 74, 81-83, or 87-88, or a pharmaceutically acceptable salt thereof, wherein
R¹⁷ is C₁-C₁₀ alkylene;
A is a bond;
RL is -AA₁-AA₂-;
AA₁ and AA₂ are each independently a proteinogenic amino acid;
Y is 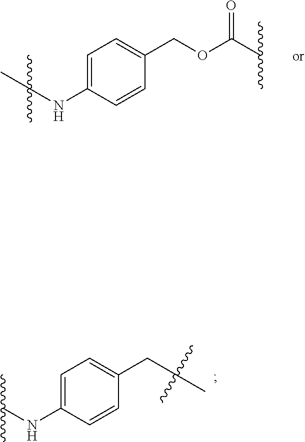 [SMILES: *Nc1ccc(COC(*)=O)cc1 ; *Cc1ccc(N*)cc1]
and
the wavy lines indicate the site of attachment to the rest of the Ligand-Drug Conjugate compound or pharmaceutically acceptable salt thereof.

90. The Ligand-Drug Conjugate compound of any one of claims 66-67, 70-71, 74, 81-83, or 87-89, or a pharmaceutically acceptable salt thereof, wherein
A is a bond;
RL is 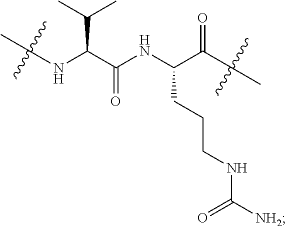 [SMILES: *N[C@H](C(=O)N[C@@H](CCCNC(N)=O)C(*)=O)C(C)C]
and
Y is

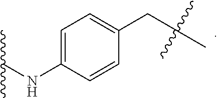 [SMILES: *Cc1ccc(N*)cc1]

91. The Ligand-Drug Conjugate compound of any one of claims 66-67, 70-71, 74, 81-83, or 87-90, or a pharmaceutically acceptable salt thereof, wherein Y-D is 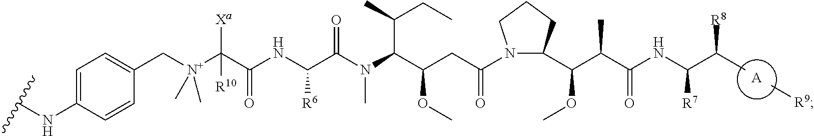
and
the wavy line indicates the site of attachment to the rest of the Ligand-Drug Conjugate compound or pharmaceutically acceptable salt thereof.

92. The Ligand-Drug Conjugate compound of any one of claims 66-67, 70-71, 74, 81-83, or 87-90, or a pharmaceutically acceptable salt thereof, wherein Y-D is 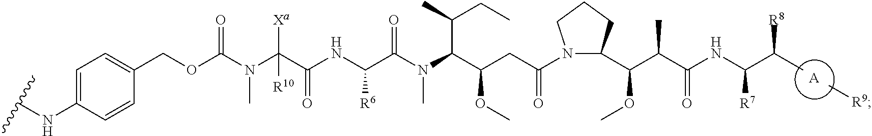
and
the wavy line indicates the site of attachment to the rest of the Ligand-Drug Conjugate compound or pharmaceutically acceptable salt thereof.

93. The Ligand-Drug Conjugate compound of any one of claims 66-67, 70-71, 74, 81-83, or 87-91, wherein the compound is 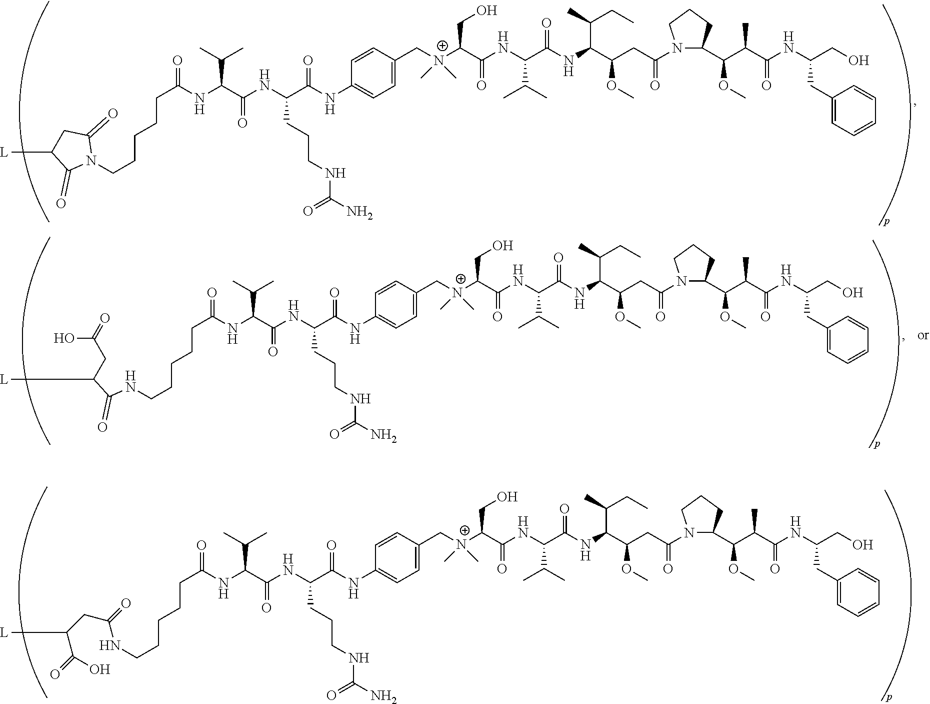
or a pharmaceutically acceptable salt thereof.

94. The Ligand-Drug Conjugate compound of any one of claims 66-67, 70-71, 74, 81-83, 87-90, or 92, wherein the compound is 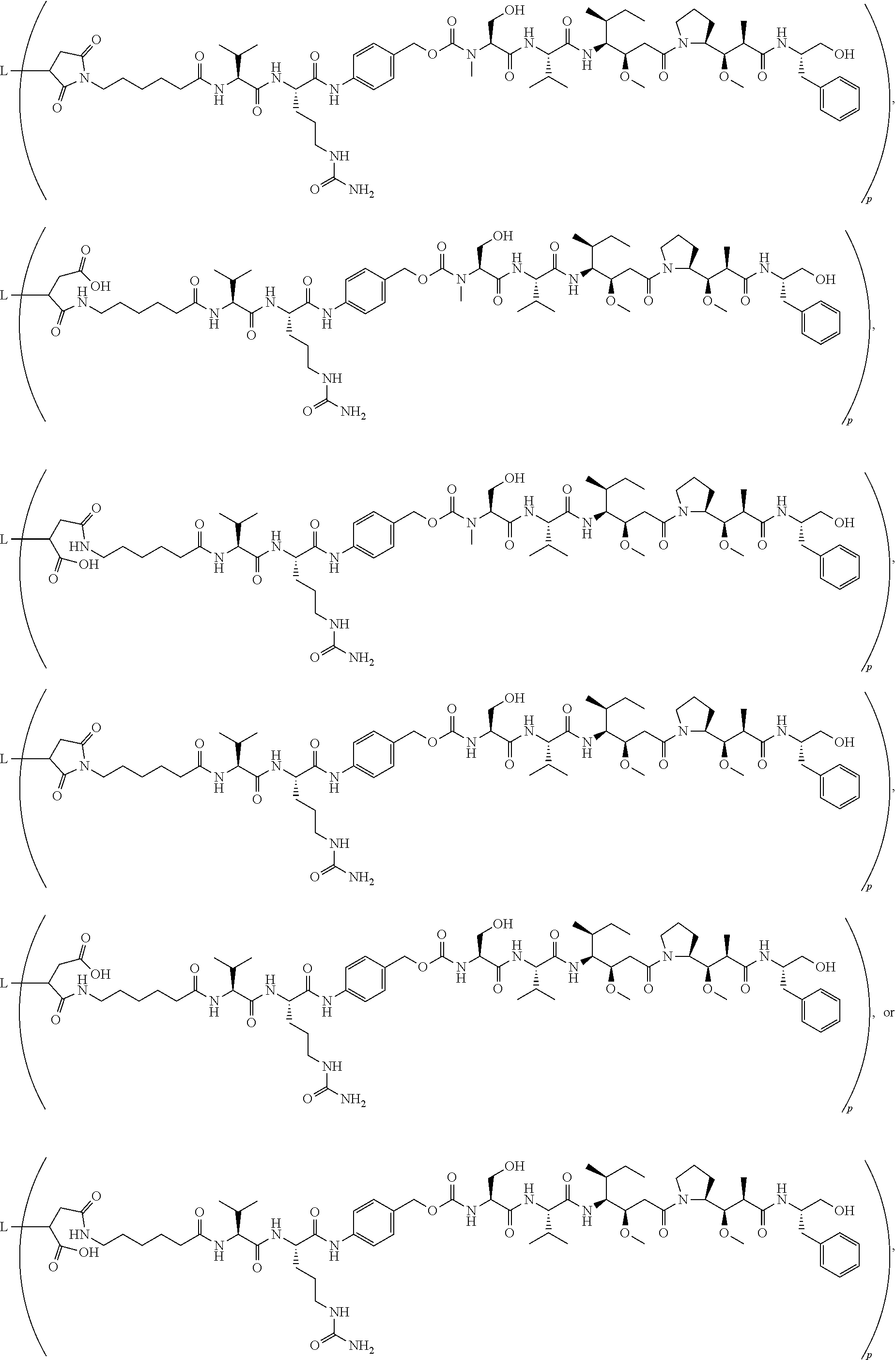
or a pharmaceutically acceptable salt thereof.

95. The Ligand-Drug Conjugate compound of any one of claims 66-94, wherein p is an integer ranging from 2 to 8.

96. The Ligand-Drug Conjugate compound of any one of claims 66-95, wherein p is 4.

97. A pharmaceutical composition comprising the Ligand-Drug Conjugate compound of any one of claims 66-94 and a pharmaceutically acceptable excipient.

98. The pharmaceutical composition of claim 97, wherein the composition comprises a plurality of Ligand-Drug Conjugate compounds with an average drug-loading from 2 to 8.

99. The pharmaceutical composition of claim 98, wherein the average drug loading is about 4.

100. The pharmaceutical composition claim 98, wherein the average drug loading is from 3.5 to 4.5.

101. A method of treating cancer comprising administering a therapeutically effective amount of the Ligand-Drug Conjugate compound of any one of claims 66-96, or a pharmaceutically acceptable salt thereof, to a subject in need thereof.

102. The method of claim 101, wherein the subject tolerates treatment from the Ligand-Drug Conjugate compound better than another Ligand-Drug Conjugate compound in therapeutically effective doses.

103. The method of claim 102, wherein the another Ligand-Drug Conjugate compound comprises a monomethyl auristatin E or monomethyl auristatin F Drug Unit.

104. A compound, wherein the compound is selected from the group consisting of the compounds listed in Table 1 or a salt thereof.

105. A Drug-Linker compound, wherein the Drug-Linker compound is selected from the group consisting of the compounds listed in Table 2 or a salt thereof.

106. A Ligand-Drug Conjugate compound, wherein the Ligand-Drug Conjugate compound is selected from the group consisting of the compounds listed in Table 3, or a salt thereof, and p is an integer ranging from 1 to 12.

107. The Ligand-Drug Conjugate compound of any one of claims 66-96 or 106, wherein L is an antibody.

108. The Ligand-Drug Conjugate compound of any one of claims 66-96 or 106-107, wherein L comprises an amino acid sequence that is at least 80% identical to the amino acid sequence of any one of SEQ ID Nos. 1 to 1044.

109. The Ligand Drug-Conjugate of any one of claims 66-96 or 106-108, wherein L comprises an amino acid sequence that is at least 85% identical to the amino acid sequence of any one of SEQ ID Nos. 1 to 1044.

110. The Ligand Drug-Conjugate of any one of claims 66-96 or 106-109, wherein L comprises an amino acid sequence that is at least 90% identical to the amino acid sequence of any one of SEQ ID Nos. 1 to 1044.

111. The Ligand Drug-Conjugate of any one of claims 66-96 or 106-110, wherein L comprises an amino acid sequence that is at least 95% identical to the amino acid sequence of any one of SEQ ID Nos. 1 to 1044.

112. The Ligand Drug-Conjugate of any one of claims 66-96 or 106-111, wherein L comprises an amino acid sequence that is at least 98% identical to the amino acid sequence of any one of SEQ ID Nos. 1 to 1044.

113. The Ligand Drug-Conjugate of any one of claims 66-96 or 106-112, wherein L comprises an amino acid sequence that is at least 99% identical to the amino acid sequence of any one of SEQ ID Nos. 1 to 1044.

114. The Ligand-Drug Conjugate compound of any one of claims claim 66-96 or 106-113, wherein L comprises the amino acid sequence of any one of SEQ ID Nos. 1 to 1044.

115. The Ligand-Drug Conjugate compound of any one of claims 66-96 or 106-114, wherein L consists of the amino acid sequence of any one of SEQ ID Nos. 1 to 1044.

116. The Ligand-Drug Conjugate of any one of claims 66-96 or 106-115, wherein p is 8.

117. The Ligand-Drug Conjugate of any one of claims 66-96 or 106-115, wherein p is and integer from 1 to 8.

118. A Ligand-Drug Conjugate of the following formula: 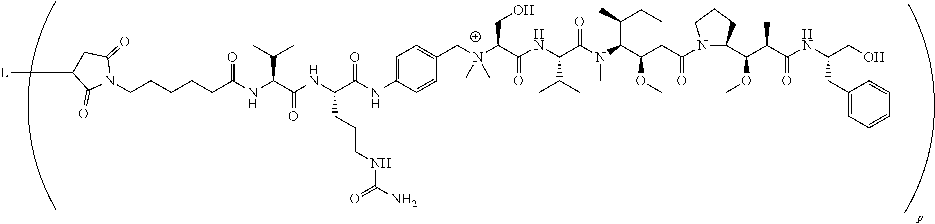
wherein L is an anti-gpNMB antibody,
and p is an integer from 1 to 14, or from 1 to 8, or is 8.

119. The Ligand-Drug Conjugate of claim 118, wherein the anti-gpNMB antibody comprises an amino acid sequence that is at least 80, 85, 90, 95, 98, or 99% identical to the amino acid sequence of any one of SEQ ID Nos. 935-944.


SEQUENCE LISTING (1044 sequences)

SEQ ID NO: 1 - protein, 453 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 452 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 453 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 452 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 213 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 453 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 452 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 453 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 9 - protein, 452 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 10 - protein, 213 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 11 - protein, 453 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPCVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 12 - protein, 452 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPCVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 13 - protein, 5 aa, synthetic construct
TAAIS

SEQ ID NO: 14 - protein, 17 aa, synthetic construct
GIIPIFGKAHYAQKFQG

SEQ ID NO: 15 - protein, 14 aa, synthetic construct
KFHFVSGSPFGMDV

SEQ ID NO: 16 - protein, 11 aa, synthetic construct
RASQSVSSYLA

SEQ ID NO: 17 - protein, 7 aa, synthetic construct
DASNRAT

SEQ ID NO: 18 - protein, 8 aa, synthetic construct
QQRSNWPT

SEQ ID NO: 19 - protein, 123 aa, synthetic construct
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 20 - protein, 106 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 21 - protein, 5 aa, synthetic construct
HYMMA

SEQ ID NO: 22 - protein, 17 aa, synthetic construct
RIGPSGGPTHYADSVKG

SEQ ID NO: 23 - protein, 19 aa, synthetic construct
YDSGYDYVAVAGPAEYFQH

SEQ ID NO: 24 - protein, 11 aa, synthetic construct
RASQSISTWLA

SEQ ID NO: 25 - protein, 7 aa, synthetic construct
KASNLHT

SEQ ID NO: 26 - protein, 9 aa, synthetic construct
QQYNSYSRT

SEQ ID NO: 27 - protein, 128 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSS

SEQ ID NO: 28 - protein, 107 aa, synthetic construct
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIK

SEQ ID NO: 29 - protein, 458 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 457 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 214 aa, synthetic construct
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 458 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 33 - protein, 457 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 34 - protein, 214 aa, synthetic construct
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 35 - protein, 458 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 36 - protein, 457 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 37 - protein, 214 aa, synthetic construct; identical to SEQ ID NO: 34

SEQ ID NO: 38 - protein, 5 aa, synthetic construct
DYYIT

SEQ ID NO: 39 - protein, 17 aa, synthetic construct
WIYPGSGNTKYNEKFKG

SEQ ID NO: 40 - protein, 8 aa, synthetic construct
YGNYWFAY

SEQ ID NO: 41 - protein, 15 aa, synthetic construct
KASQSVDFDGDSYMN

SEQ ID NO: 42 - protein, 7 aa, synthetic construct
AASNLES

SEQ ID NO: 43 - protein, 9 aa, synthetic construct
QQSNEDPWT

SEQ ID NO: 44 - protein, 117 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 45 - protein, 111 aa, synthetic construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 46 - protein, 447 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 446 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 218 aa, synthetic construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 5 aa, synthetic construct
SGYWN

SEQ ID NO: 50 - protein, 16 aa, synthetic construct
YISDSGITYYNPSLKS

SEQ ID NO: 51 - protein, 11 aa, synthetic construct
RTLATYYAMDY

SEQ ID NO: 52 - protein, 16 aa, synthetic construct
RASQSLVHSDGNTYLH

SEQ ID NO: 53 - protein, 7 aa, synthetic construct
RVSNRFS

SEQ ID NO: 54 - protein, 9 aa, synthetic construct
SQSTHVPPT

SEQ ID NO: 55 - protein, 119 aa, synthetic construct
QVQLQESGPGLVKPSETLSLTCTVSGDSITSGYWNWIRQPPGKGLEYIGYISDSGITYYNPSLKSRVTISRDTSKNQYSLKLSSVTAADTAVYYCARRTLATYYAMDYWGQGTLVTVSS

SEQ ID NO: 56 - protein, 112 aa, synthetic construct
DFVMTQSPLSLPVTLGQPASISCRASQSLVHSDGNTYLHWYQQRPGQSPRLLIYRVSNRFSGVPDRFSGSGSGTDFTLKISRVEAEDVGVYYCSQSTHVPPTFGQGTKLEIK

SEQ ID NO: 57 - protein, 449 aa, synthetic construct
QVQLQESGPGLVKPSETLSLTCTVSGDSITSGYWNWIRQPPGKGLEYIGYISDSGITYYNPSLKSRVTISRDTSKNQYSLKLSSVTAADTAVYYCARRTLATYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 448 aa, synthetic construct
QVQLQESGPGLVKPSETLSLTCTVSGDSITSGYWNWIRQPPGKGLEYIGYISDSGITYYNPSLKSRVTISRDTSKNQYSLKLSSVTAADTAVYYCARRTLATYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 219 aa, synthetic construct
DFVMTQSPLSLPVTLGQPASISCRASQSLVHSDGNTYLHWYQQRPGQSPRLLIYRVSNRFSGVPDRFSGSGSGTDFTLKISRVEAEDVGVYYCSQSTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 449 aa, synthetic construct
QVQLQESGPGLVKPSETLSLTCTVSGDSITSGYWNWIRQPPGKGLEYIGYISDSGITYYNPSLKSRVTISRDTSKNQYSLKLSSVTAADTAVYYCARRTLATYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 448 aa, synthetic construct
QVQLQESGPGLVKPSETLSLTCTVSGDSITSGYWNWIRQPPGKGLEYIGYISDSGITYYNPSLKSRVTISRDTSKNQYSLKLSSVTAADTAVYYCARRTLATYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 219 aa, synthetic construct; identical to SEQ ID NO: 59

SEQ ID NO: 63 - protein, 5 aa, synthetic construct
DYNVN

SEQ ID NO: 64 - protein, 17 aa, synthetic construct
VINPKYGTTRYNQKFKG

SEQ ID NO: 65 - protein, 7 aa, synthetic construct
GLNAWDY

SEQ ID NO: 66 - protein, 11 aa, synthetic construct
GASENIYGALN

SEQ ID NO: 67 - protein, 7 aa, synthetic construct
GATNLED

SEQ ID NO: 68 - protein, 9 aa, synthetic construct
QNVLTTPYT

SEQ ID NO: 69 - protein, 116 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSS

SEQ ID NO: 70 - protein, 107 aa, synthetic construct
DIQMTQSPSSLSASVGDRVTITCGASENIYGALNWYQQKPGKAPKLLIYGATNLEDGVPSRFSGSGSGRDYTFTISSLQPEDIATYYCQNVLTTPYTFGQGTKLEIK

SEQ ID NO: 71 - protein, 446 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 445 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 214 aa, synthetic construct
DIQMTQSPSSLSASVGDRVTITCGASENIYGALNWYQQKPGKAPKLLIYGATNLEDGVPSRFSGSGSGRDYTFTISSLQPEDIATYYCQNVLTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 446 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 445 aa, synthetic construct
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 214 aa, synthetic construct; identical to SEQ ID NO: 73

SEQ ID NO: 77 - protein, 7 aa, synthetic construct
SGSYYWG

SEQ ID NO: 78 - protein, 16 aa, synthetic construct
NIYYSGSTYYNPSLRS

SEQ ID NO: 79 - protein, 10 aa, synthetic construct
EGSYPNQFDP

SEQ ID NO: 80 - protein, 11 aa, synthetic construct
RASQSVSSNLA

SEQ ID NO: 81 - protein, 7 aa, synthetic construct
GASTRAT

SEQ ID NO: 82 - protein, 9 aa, synthetic construct
QQYHSFPFT

SEQ ID NO: 83 - protein, 120 aa, synthetic construct
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSS

SEQ ID NO: 84 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYHSFPFTFGGGTKVEIK

SEQ ID NO: 85 - protein, 450 aa, synthetic construct
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 449 aa, synthetic construct
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 87 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYHSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 450 aa, synthetic construct
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 449 aa, synthetic construct
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - protein, 214 aa, synthetic construct; identical to SEQ ID NO: 87

SEQ ID NO: 91 - protein, 11 aa, synthetic construct
GSISSSSYYWG

SEQ ID NO: 92 - protein, 16 aa, synthetic construct
NIYYSGSTYYNPSLKS

SEQ ID NO: 93 - protein, 12 aa, synthetic construct
AREGSYPNWFDP

SEQ ID NO: 94 - protein, 11 aa, synthetic construct
RASQSVSSNLA

SEQ ID NO: 95 - protein, 7 aa, synthetic construct
GASTRAT

SEQ ID NO: 96 - protein, 9 aa, synthetic construct
QQYHSFPFT

SEQ ID NO: 97 - protein, 120 aa, synthetic construct
QLQLQESGPGLVKPSETLSLTCTVSGGSISSSSYYWGWIRQPPGKGLEWIGNIYYSGSTYYNPSLKSRVTISVDTSKNQFSLKLSSVTAADTAVYYCAREGSYPNWFDPWGQGTLVTVSS

SEQ ID NO: 98 - protein, 107 aa, synthetic construct; identical to SEQ ID NO: 84

SEQ ID NO: 99 - protein, 11 aa, synthetic construct
GSIKSGSHYWG

SEQ ID NO: 100 - protein, 16 aa, synthetic construct
NIYYSGSTYYNPSLRS

SEQ ID NO: 101 - protein, 12 aa, synthetic construct
AREGSYPNWFDP

SEQ ID NO: 102 - protein, 11 aa, synthetic construct
RASQSVSSNLA

SEQ ID NO: 103 - protein, 7 aa, synthetic construct
GASTRAT

SEQ ID NO: 104 - protein, 9 aa, synthetic construct
QQYHSFPFT

SEQ ID NO: 105 - protein, 120 aa, synthetic construct
QLQLQESGPGLVKPSETLSLTCTVSGGSIKSGSHYWGWIRQPPGKGLEWIGNIYYSGSTYYNPSLRSRVTISVDTSKNQFSLKLSSVTAADTAVYYCAREGSYPNWFDPWGQGTLVTVSS

SEQ ID NO: 106 - protein, 107 aa, synthetic construct; identical to SEQ ID NO: 84

SEQ ID NO: 107 - protein, 11 aa, synthetic construct
GSIKSGSHYWG

SEQ ID NO: 108 - protein, 16 aa, synthetic construct
NIYYSGSTYYNPSLKS

SEQ ID NO: 109 - protein, 12 aa, synthetic construct
AREGSYPNWLDP

SEQ ID NO: 110 - protein, 11 aa, synthetic construct
RASQSVSSNLA

SEQ ID NO: 111 - protein, 7 aa, synthetic construct
GASTRAT

SEQ ID NO: 112 - protein, 9 aa, synthetic construct
QQYHSFPFT

SEQ ID NO: 113 - protein, 120 aa, synthetic construct
QLQLQESGPGLVKPSETLSLTCTVSGGSIKSGSHYWGWIRQPPGKGLEWIGNIYYSGSTYYNPSLKSRVTISVDTSKNQFSLKLSSVTAADTAVYYCAREGSYPNWLDPWGQGTLVTVSS

SEQ ID NO: 114 - protein, 107 aa, synthetic construct; identical to SEQ ID NO: 84

SEQ ID NO: 115 - protein, 11 aa, synthetic construct
GSIKSGSYYWG

SEQ ID NO: 116 - protein, 16 aa, synthetic construct
NIYYSGSTYYNPSLKS

SEQ ID NO: 117 - protein, 12 aa, synthetic construct
AREGSYPNQFDP

SEQ ID NO: 118 - protein, 11 aa, synthetic construct
RASQSVSSNLA

SEQ ID NO: 119 - protein, 7 aa, synthetic construct
GASTRAT

SEQ ID NO: 120 - protein, 9 aa, synthetic construct
QQYHSFPFT

SEQ ID NO: 121 - protein, 120 aa, synthetic construct
QLQLQESGPGLVKPSETLSLTCTVSGGSIKSGSYYWGWIRQPPGKGLEWIGNIYYSGSTYYNPSLKSRVTISVDTSKNQFSLKLSSVTAADTAVYYCAREGSYPNQFDPWGQGILVTVSS

SEQ ID NO: 122 - protein, 107 aa, synthetic construct; identical to SEQ ID NO: 84

SEQ ID NO: 123 - protein, 11 aa, synthetic construct
GSIKSGSHYWG

SEQ ID NO: 124 - protein, 16 aa, synthetic construct
NIYYSGSTYYNPSLKS

SEQ ID NO: 125 - protein, 12 aa, synthetic construct
AREGSYPNWFDP

SEQ ID NO: 126 - protein, 11 aa, synthetic construct
RASQSVSTNLA

SEQ ID NO: 127 - protein, 7 aa, synthetic construct
DASARVT

SEQ ID NO: 128 - protein, 9 aa, synthetic construct
QQYHSFPFT

SEQ ID NO: 129 - protein, 120 aa, synthetic construct
QLQLQESGPGLVKPSETLSLTCTVSGGSIKSGSHYWGWIRQPPGKGLEWIGNIYYSGSTYYNPSLKSRVTMSVDTSKNQFSLKLSSVTAADTAVYYCAREGSYPNWFDPWGQGTLVTVSS

SEQ ID NO: 130 - protein, 107 aa, synthetic construct
EIVMTQSPATLSVSPGERATLSCRASQSVSTNLAWYQQKPGQAPRLLIYDASARVTGIPARFSGSGSGTEFTLTISSLQSEDFAVYYCQQYHSFPFTFGGGTKVEIK

SEQ ID NO: 131 - protein, 11 aa, synthetic construct
GSISSSSYYWG

SEQ ID NO: 132 - protein, 16 aa, synthetic construct
NIYYSGSTYYNPSLKS

SEQ ID NO: 133 - protein, 12 aa, synthetic construct
AREGSYTTVLNV

SEQ ID NO: 134 - protein, 12 aa, synthetic construct
RASQSVSSSYLA

SEQ ID NO: 135 - protein, 7 aa, synthetic construct
GASSRAT

SEQ ID NO: 136 - protein, 9 aa, synthetic construct
QQAASYPLT

SEQ ID NO: 137 - protein, 120 aa, synthetic construct
QLQLQESGPGLVKPSETLSLTCTVSGGSISSSSYYWGWIRQPPGKGLEWIGNIYYSGSTYYNPSLKSRVTISVDTSKNQFSLKLSSVTAADTAVYYCAREGSYTTVLNVWGQGTMVTVSS

SEQ ID NO: 138 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQAASYPLTFGGGTKVEIK

SEQ ID NO: 139 - protein, 11 aa, synthetic construct
GSIGRGSYYWG

SEQ ID NO: 140 - protein, 16 aa, synthetic construct
NIYYSGSTYYNPSLKS

SEQ ID NO: 141 - protein, 12 aa, synthetic construct
AREGSYTTVLNV

SEQ ID NO: 142 - protein, 12 aa, synthetic construct
RASQSVASSHLA

SEQ ID NO: 143 - protein, 7 aa, synthetic construct
DAVSRAT

SEQ ID NO: 144 - protein, 9 aa, synthetic construct
QQAASYPLT

SEQ ID NO: 145 - protein, 120 aa, synthetic construct
QLQLQESGPGLVKPSETLSLTCTVSGGSIGRGSYYWGWIRQPPGKGLEWIGNIYYSGSTYYNPSLKSRVTISVDTSKNQFSLKLSSVTAADTAVYYCAREGSYTTVLNVWGQGTMVTVSS

SEQ ID NO: 146 - protein, 108 aa, synthetic construct
EIVLTQSPGTLSLSPGERATLSCRASQSVASSHLAWYQQKPGQAPRLLIYDAVSRATGIPDRFSGSGSGTDFTLTISRLEPEDFAVYYCQQAASYPLTFGGGTKVEIK

SEQ ID NO: 147 - protein, 11 aa, synthetic construct
GSISSGGYYWS

SEQ ID NO: 148 - protein, 16 aa, synthetic construct
NIYYSGSTYYNPSLKS

SEQ ID NO: 149 - protein, 13 aa, synthetic construct
ARESSTISADFDL

SEQ ID NO: 150 - protein, 11 aa, synthetic construct
RASQGISRWLA

SEQ ID NO: 151 - protein, 7 aa, synthetic construct
AASSLQS

SEQ ID NO: 152 - protein, 9 aa, synthetic construct
QQAHTFPYT

SEQ ID NO: 153 - protein, 121 aa, synthetic construct
QVQLQESGPGLVKPSQTLSLTCTVSGGSISSGGYYWSWIRQHPGKGLEWIGNIYYSGSTYYNPSLKSRVTISVDTSKNQFSLKLSSVTAADTAVYYCARESSTISADFDLWGRGTLVTVSS

SEQ ID NO: 154 - protein, 107 aa, synthetic construct
DIQMTQSPSSVSASVGDRVTITCRASQGISRWLAWYQQKPGKAPKLLIYAASSLQSGVPSRFSGSGSGTDFTLTISSLQPEDFATYYCQQAHTFPYTFGGGTKVEIK

SEQ ID NO: 155 - protein, 11 aa, synthetic construct
GSISHGGYYWS

SEQ ID NO: 156 - protein, 16 aa, synthetic construct
NIYYSGSTYYNPSLKS

SEQ ID NO: 157 - protein, 13 aa, synthetic construct
ARESSTISADFDL

SEQ ID NO: 158 - protein, 11 aa, synthetic construct
RASQGISRWLA

SEQ ID NO: 159 - protein, 7 aa, synthetic construct
AASSLQS

SEQ ID NO: 160 - protein, 9 aa, synthetic construct
QQAHTFPYT

SEQ ID NO: 161 - protein, 121 aa, synthetic construct
QLQLQESGPGLVKPSETLSLTCTASGGSISHGGYYWSWIRQHPGKGLEWIGNIYYSGSTYYNPSLKSRVTMSVDTSKNQFSLKLSSVTAADTAVYYCARESSTISADFDLWGRGTLVTVSS

SEQ ID NO: 162 - protein, 107 aa, synthetic construct; identical to SEQ ID NO: 154

SEQ ID NO: 163 - protein, 11 aa, synthetic construct
GSISSGGYYWS

SEQ ID NO: 164 - protein, 16 aa, synthetic construct
NIYYSGSTYYNPSLKS

SEQ ID NO: 165 - protein, 13 aa, synthetic construct
ARGLSTIDEAFDP

SEQ ID NO: 166 - protein, 11 aa, synthetic construct
RASQSISSWLA

SEQ ID NO: 167 - protein, 7 aa, synthetic construct
KASSLES

SEQ ID NO: 168 - protein, 9 aa, synthetic construct
QQDNSYPYT

SEQ ID NO: 169 - protein, 121 aa, synthetic construct
QVQLQESGPGLVKPSQTLSLTCTVSGGSISSGGYYWSWIRQHPGKGLEWIGNIYYSGSTYYNPSLKSRVTISVDTSKNQFSLKLSSVTAADTAVYYCARGLSTIDEAFDPWGQGTLVTVSS

SEQ ID NO: 170 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQDNSYPYTFGGGTKVEIK

SEQ ID NO: 171 - protein, 11 aa, synthetic construct
GSISDGSYYWS

SEQ ID NO: 172 - protein, 16 aa, synthetic construct
NIYYSGSTYYNPSLRS

SEQ ID NO: 173 - protein, 13 aa, synthetic construct
ARGLSTIDEAFDP

SEQ ID NO: 174 - protein, 11 aa, synthetic construct
RASQSISSWLA

SEQ ID NO: 175 - protein, 7 aa, synthetic construct
KASSLES

SEQ ID NO: 176 - protein, 9 aa, synthetic construct
QQDNSYPYT

SEQ ID NO: 177 - protein, 121 aa, synthetic construct
QLQLQESGPGLVKPSETLSLTCTVSGGSISDGSYYWSWIRQHPGKGLEWIGNIYYSGSTYYNPSLRSRVTMSVDTSKNQFSLKLSSVTAADTAVYYCARGLSTIDEAFDPWGQGTLVTVSS

SEQ ID NO: 178 - protein, 107 aa, synthetic construct; identical to SEQ ID NO: 170

SEQ ID NO: 179 - protein, 11 aa, synthetic construct
GSISDGSYYWS

SEQ ID NO: 180 - protein, 16 aa, synthetic construct
NIYYSGSTYYNPSLRS

SEQ ID NO: 181 - protein, 13 aa, synthetic construct
ARGLSTIDEAFDP

SEQ ID NO: 182 - protein, 11 aa, synthetic construct
RASKSISSWLA

SEQ ID NO: 183 - protein, 7 aa, synthetic construct
EASSLHS

SEQ ID NO: 184 - protein, 9 aa, synthetic construct
QQDNSYPYT

SEQ ID NO: 185 - protein, 121 aa, synthetic construct
QVQLQESGPGLVKPSQTLSLTCTVSGGSISDGSYYWSWIRQHPGKGLEWIGNIYYSGSTYYNPSLRSRVTMSVDTSKNQFSLKLSSVTAADTAVYYCARGLSTIDEAFDPWGQGTLVTVSS

SEQ ID NO: 186 - protein, 107 aa, synthetic construct
DIQMTQSPSTLSASVGDRVTITCRASKSISSWLAWYQQKPGKAPKLLIYEASSLHSGVPSRFSGSGSGTEFTLTISSLQPDDFATYYCQQDNSYPYTFGGGTKVEIK

SEQ ID NO: 187 - protein, 9 aa, synthetic construct
GSISSYYWS

SEQ ID NO: 188 - protein, 16 aa, synthetic construct
YIYSSGSTNYNPSLKS

SEQ ID NO: 189 - protein, 15 aa, synthetic construct
ARGSGQYAAPDYGMD

SEQ ID NO: 190 - protein, 11 aa, synthetic construct
RASQSISSWLA

SEQ ID NO: 191 - protein, 7 aa, synthetic construct
KASSLES

SEQ ID NO: 192 - protein, 9 aa, synthetic construct
QQDNSFPFT

SEQ ID NO: 193 - protein, 122 aa, synthetic construct
QVQLQESGPGLVKPSETLSLTCTVSGGSISSYYWSWIRQPPGKGLEWIGYIYSSGSTNYNPSLKSRVTISVDTSKNQFSLKLSSVTAADTAVYYCARGSGQYAAPDYGMDVWGQGTTVTVSS

SEQ ID NO: 194 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQDNSFPFTFGGGTKVEIK

SEQ ID NO: 195 - protein, 9 aa, synthetic construct
GSIISYYWG

SEQ ID NO: 196 - protein, 16 aa, synthetic construct
YIYSSGSTSYNPSLKS

SEQ ID NO: 197 - protein, 16 aa, synthetic construct
ARGSGLYAAPDYGLDV

SEQ ID NO: 198 - protein, 11 aa, synthetic construct
RASQSISSWLA

SEQ ID NO: 199 - protein, 7 aa, synthetic construct
KASSLES

SEQ ID NO: 200 - protein, 9 aa, synthetic construct
QQDNSFPFT

SEQ ID NO: 201 - protein, 122 aa, synthetic construct
QVQLQESGPGLVKPSETLSLTCTVSGGSIISYYWGWIRQPPGKGLEWIGYIYSSGSTSYNPSLKSRVTISVDTSKNQFSLKLSSVTAADTAVYYCARGSGLYAAPDYGLDVWGQGTTVTVSS

SEQ ID NO: 202 - protein, 107 aa, synthetic construct; identical to SEQ ID NO: 194

SEQ ID NO: 203 - protein, 9 aa, synthetic construct
FTFSSYAMS

SEQ ID NO: 204 - protein, 17 aa, synthetic construct
TISGSGGSTYYADSVKG

SEQ ID NO: 205 - protein, 13 aa, synthetic construct
ARGAGHYDLVGRY

SEQ ID NO: 206 - protein, 11 aa, synthetic construct
RASQSISSYLN

SEQ ID NO: 207 - protein, 7 aa, synthetic construct
AASSLQS

SEQ ID NO: 208 - protein, 9 aa, synthetic construct
QQLYSLPPT

SEQ ID NO: 209 - protein, 120 aa, synthetic construct
EVQLLESGGGLVQPGGSLRLSCAASGFTFSSYAMSWVRQAPGKGLEWVSTISGSGGSTYYADSVKGRFTISRDNSKNTLYLQMNSLRAEDTAVYYCARGAGHYDLVGRYWGQGTLVTVSS

SEQ ID NO: 210 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQLYSLPPTFGGGTKVEIK

SEQ ID NO: 211 - protein, 9 aa, synthetic construct
FTFSSYAMS

SEQ ID NO: 212 - protein, 17 aa, synthetic construct
AISGSGGSTYYADSVKG

SEQ ID NO: 213 - protein, 10 aa, synthetic construct
ARVGFRALNY

SEQ ID NO: 214 - protein, 11 aa, synthetic construct
RASQDISSWLA

SEQ ID NO: 215 - protein, 7 aa, synthetic construct
AASSLQS

SEQ ID NO: 216 - protein, 10 aa, synthetic construct
QQATSYPPWT

SEQ ID NO: 217 - protein, 117 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARVGFRALNYWGQGTTVTVSS

SEQ ID NO: 218 - protein, 108 aa, synthetic construct
DIQLTQSPSSVSASVGDRVTITCRASQDISSWLAWYQQKPGKAPKLLIYAASSLQSGVPSRFSGSGSGTDFTLTISSLQPEDFATYYCQQATSYPPWTFGGGTKVEIK

SEQ ID NO: 219 - protein, 9 aa, synthetic construct
GTFSSYAIS

SEQ ID NO: 220 - protein, 17 aa, synthetic construct
GIIPIFGTASYAQKFQG

SEQ ID NO: 221 - protein, 13 aa, synthetic construct
ARQQYDGRRYFGL

SEQ ID NO: 222 - protein, 11 aa, synthetic construct
RASQSVSSNLA

SEQ ID NO: 223 - protein, 7 aa, synthetic construct
SASTRAT

SEQ ID NO: 224 - protein, 9 aa, synthetic construct
QQVNVWPPT

SEQ ID NO: 225 - protein, 120 aa, synthetic construct
QVQLVQSGAEVKKPGSSVKVSCKASGGTFSSYAISWVRQAPGQGLEWMGGIIPIFGTASYAQKFQGRVTITADESTSTAYMELSSLRSEDTAVYYCARQQYDGRRYFGLWGRGTLVTVSS

SEQ ID NO: 226 - protein, 107 aa, synthetic construct
EIVMTQSPATLSVSPGERATLSCRASQSVSSNLAWYQQKPGQAPRLLIYSASTRATGIPARFSGSGSGTEFTLTISSLQSEDFAVYYCQQVNVWPPTFGGGTKVEIK

SEQ ID NO: 227 - protein, 9 aa, synthetic construct
GTFSSYAIS

SEQ ID NO: 228 - protein, 17 aa, synthetic construct
GIIPIFGTANYAQKFQG

SEQ ID NO: 229 - protein, 9 aa, synthetic construct
ARGGPWFDP

SEQ ID NO: 230 - protein, 11 aa, synthetic construct
RASQSISSWLA

SEQ ID NO: 231 - protein, 7 aa, synthetic construct
KASSLES

SEQ ID NO: 232 - protein, 10 aa, synthetic construct
QQYNSYPPFT

SEQ ID NO: 233 - protein, 116 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGGPWFDPWGQGTLVTVSS

SEQ ID NO: 234 - protein, 108 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPPFTFGGGTKVEIK

SEQ ID NO: 235 - protein, 9 aa, synthetic construct
FTFSSYAMS

SEQ ID NO: 236 - protein, 17 aa, synthetic construct
AISGSGGSTSYADSVKG

SEQ ID NO: 237 - protein, 13 aa, synthetic construct
AKPSLATMLAFDI

SEQ ID NO: 238 - protein, 11 aa, synthetic construct
RASQSISSWLA

SEQ ID NO: 239 - protein, 7 aa, synthetic construct
DASSLES

SEQ ID NO: 240 - protein, 9 aa, synthetic construct
QQSKSYPRT

SEQ ID NO: 241 - protein, 120 aa, synthetic construct
EVQLLESGGGLVQPGGSLRLSCAASGFTFSSYAMSWVRQAPGKGLEWVSAISGSGGSTSYADSVKGRFTISRDNSKNTLYLQMNSLRAEDTAVYYCAKPSLATMLAFDIWGQGTMVTVSS

SEQ ID NO: 242 - protein, 107 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQSKSYPRTFGGGTKVEIK

SEQ ID NO: 243 - protein, 11 aa, synthetic construct
GSISSSVYYWS

SEQ ID NO: 244 - protein, 16 aa, synthetic construct
SILVSGSTYYNPSLKS

SEQ ID NO: 245 - protein, 9 aa, synthetic construct
ARAVSFLDV

SEQ ID NO: 246 - protein, 11 aa, synthetic construct
RASQSISSYLN

SEQ ID NO: 247 - protein, 7 aa, synthetic construct
GASSLQS

SEQ ID NO: 248 - protein, 9 aa, synthetic construct
QQSYDPPWT

SEQ ID NO: 249 - protein, 117 aa, synthetic construct
QLQLQESGPGLVKPSETLSLTCTVSGGSISSSVYYWSWIRQPPGKGLEWIGSILVSGSTYYNPSLKSRVTISVDTSKNQFSLKLSSVTAADTAVYYCARAVSFLDVWGQGTMVIVSS

SEQ ID NO: 250 - protein, 107 aa, synthetic construct
DIQMTQSPSSLSASVGDRVTITCRASQSISSYLNWYQQKPGKAPKLLIYGASSLQSGVPSRFSGSGSGTDFTLTISSLQPEDFATYYCQQSYDPPWTFGGGTKVEIK

SEQ ID NO: 251 - protein, 450 aa, synthetic construct
QLQLQESGPGLVKPSETLSLTCTVSGGSISSSSYYWGWIRQPPGKGLEWIGNIYYSGSTYYNPSLKSRVTISVDTSKNQFSLKLSSVTAADTAVYYCAREGSYPNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 252 - protein, 214 aa, synthetic construct; identical to SEQ ID NO: 87

SEQ ID NO: 253 - protein, 450 aa, synthetic construct
QLQLQESGPGLVKPSETLSLTCTVSGGSIKSGSHYWGWIRQPPGKGLEWIGNIYYSGSTYYNPSLRSRVTISVDTSKNQFSLKLSSVTAADTAVYYCAREGSYPNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 254 - protein, 214 aa, synthetic construct; identical to SEQ ID NO: 87

SEQ ID NO: 255 - protein, 450 aa, synthetic construct
QLQLQESGPGLVKPSETLSLTCTVSGGSIKSGSHYWGWIRQPPGKGLEWIGNIYYSGSTYYNPSLKSRVTISVDTSKNQFSLKLSSVTAADTAVYYCAREGSYPNWLDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 256 - protein, 214 aa, synthetic construct; identical to SEQ ID NO: 87

SEQ ID NO: 257 - protein, 450 aa, synthetic construct
QLQLQESGPGLVKPSETLSLTCTVSGGSIKSGSYYWGWIRQPPGKGLEWIGNIYYSGSTYYNPSLKSRVTISVDTSKNQFSLKLSSVTAADTAVYYCAREGSYPNQFDPWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 258 - protein, 214 aa, synthetic construct; identical to SEQ ID NO: 87

SEQ ID NO: 259 - protein, 450 aa, synthetic construct
QLQLQESGPGLVKPSETLSLTCTVSGGSIKSGSHYWGWIRQPPGKGLEWIGNIYYSGSTYYNPSLKSRVTMSVDTSKNQFSLKLSSVTAADTAVYYCAREGSYPNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 260 - protein, 214 aa, synthetic construct
EIVMTQSPATLSVSPGERATLSCRASQSVSTNLAWYQQKPGQAPRLLIYDASARVTGIPARFSGSGSGTEFTLTISSLQSEDFAVYYCQQYHSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 261 - protein, 450 aa, synthetic construct
QLQLQESGPGLVKPSETLSLTCTVSGGSISSSSYYWGWIRQPPGKGLEWIGNIYYSGSTYYNPSLKSRVTISVDTSKNQFSLKLSSVTAADTAVYYCAREGSYTTVLNVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 262 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQAASYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 263 - protein, 450 aa, synthetic construct
QLQLQESGPGLVKPSETLSLTCTVSGGSIGRGSYYWGWIRQPPGKGLEWIGNIYYSGSTYYNPSLKSRVTISVDTSKNQFSLKLSSVTAADTAVYYCAREGSYTTVLNVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 264 - protein, 215 aa, synthetic construct
EIVLTQSPGTLSLSPGERATLSCRASQSVASSHLAWYQQKPGQAPRLLIYDAVSRATGIPDRFSGSGSGTDFTLTISRLEPEDFAVYYCQQAASYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 265 - protein, 451 aa, synthetic construct
QVQLQESGPGLVKPSQTLSLTCTVSGGSISSGGYYWSWIRQHPGKGLEWIGNIYYSGSTYYNPSLKSRVTISVDTSKNQFSLKLSSVTAADTAVYYCARESSTISADFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 266 - protein, 214 aa, synthetic construct
DIQMTQSPSSVSASVGDRVTITCRASQGISRWLAWYQQKPGKAPKLLIYAASSLQSGVPSRFSGSGSGTDFTLTISSLQPEDFATYYCQQAHTFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 267 - protein, 451 aa, synthetic construct
QLQLQESGPGLVKPSETLSLTCTASGGSISHGGYYWSWIRQHPGKGLEWIGNIYYSGSTYYNPSLKSRVTMSVDTSKNQFSLKLSSVTAADTAVYYCARESSTISADFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 268 - protein, 214 aa, synthetic construct; identical to SEQ ID NO: 266

SEQ ID NO: 269 - protein, 451 aa, synthetic construct
QVQLQESGPGLVKPSQTLSLTCTVSGGSISSGGYYWSWIRQHPGKGLEWIGNIYYSGSTYYNPSLKSRVTISVDTSKNQFSLKLSSVTAADTAVYYCARGLSTIDEAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 270 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQDNSYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 271 - protein, 451 aa, synthetic construct
QLQLQESGPGLVKPSETLSLTCTVSGGSISDGSYYWSWIRQHPGKGLEWIGNIYYSGSTYYNPSLRSRVTMSVDTSKNQFSLKLSSVTAADTAVYYCARGLSTIDEAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 272 - protein, 214 aa, synthetic construct; identical to SEQ ID NO: 270

SEQ ID NO: 273 - protein, 451 aa, synthetic construct
QVQLQESGPGLVKPSQTLSLTCTVSGGSISDGSYYWSWIRQHPGKGLEWIGNIYYSGSTYYNPSLRSRVTMSVDTSKNQFSLKLSSVTAADTAVYYCARGLSTIDEAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 274 - protein, 214 aa, synthetic construct
DIQMTQSPSTLSASVGDRVTITCRASKSISSWLAWYQQKPGKAPKLLIYEASSLHSGVPSRFSGSGSGTEFTLTISSLQPDDFATYYCQQDNSYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 275 - protein, 452 aa, synthetic construct
QVQLQESGPGLVKPSETLSLTCTVSGGSISSYYWSWIRQPPGKGLEWIGYIYSSGSTNYNPSLKSRVTISVDTSKNQFSLKLSSVTAADTAVYYCARGSGQYAAPDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 276 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQDNSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 277 - protein, 452 aa, synthetic construct
QVQLQESGPGLVKPSETLSLTCTVSGGSIISYYWGWIRQPPGKGLEWIGYIYSSGSTSYNPSLKSRVTISVDTSKNQFSLKLSSVTAADTAVYYCARGSGLYAAPDYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 278 - protein, 214 aa, synthetic construct; identical to SEQ ID NO: 276

SEQ ID NO: 279 - protein, 450 aa, synthetic construct
EVQLLESGGGLVQPGGSLRLSCAASGFTFSSYAMSWVRQAPGKGLEWVSTISGSGGSTYYADSVKGRFTISRDNSKNTLYLQMNSLRAEDTAVYYCARGAGHYDLVGRYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 280 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQLYSLPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 281 - protein, 447 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARVGFRALNYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 282 - protein, 215 aa, synthetic construct
DIQLTQSPSSVSASVGDRVTITCRASQDISSWLAWYQQKPGKAPKLLIYAASSLQSGVPSRFSGSGSGTDFTLTISSLQPEDFATYYCQQATSYPPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 283 - protein, 450 aa, synthetic construct
QVQLVQSGAEVKKPGSSVKVSCKASGGTFSSYAISWVRQAPGQGLEWMGGIIPIFGTASYAQKFQGRVTITADESTSTAYMELSSLRSEDTAVYYCARQQYDGRRYFGLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 284 - protein, 214 aa, synthetic construct
EIVMTQSPATLSVSPGERATLSCRASQSVSSNLAWYQQKPGQAPRLLIYSASTRATGIPARFSGSGSGTEFTLTISSLQSEDFAVYYCQQVNVWPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 285 - protein, 446 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGGPWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 286 - protein, 215 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 287 - protein, 450 aa, synthetic construct
EVQLLESGGGLVQPGGSLRLSCAASGFTFSSYAMSWVRQAPGKGLEWVSAISGSGGSTSYADSVKGRFTISRDNSKNTLYLQMNSLRAEDTAVYYCAKPSLATMLAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 288 - protein, 214 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQSKSYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 289 - protein, 447 aa, synthetic construct
QLQLQESGPGLVKPSETLSLTCTVSGGSISSSVYYWSWIRQPPGKGLEWIGSILVSGSTYYNPSLKSRVTISVDTSKNQFSLKLSSVTAADTAVYYCARAVSFLDVWGQGTMVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 290 - protein, 214 aa, synthetic construct
DIQMTQSPSSLSASVGDRVTITCRASQSISSYLNWYQQKPGKAPKLLIYGASSLQSGVPSRFSGSGSGTDFTLTISSLQPEDFATYYCQQSYDPPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 291 - protein, 5 aa, synthetic construct
NYGMN

SEQ ID NO: 292 - protein, 17 aa, synthetic construct
WINTYTGEPTYADAFKG

SEQ ID NO: 293 - protein, 9 aa, synthetic construct
DYGDYGMDY

SEQ ID NO: 294 - protein, 15 aa, synthetic construct
RASKSVSTSGYSFMH

SEQ ID NO: 295 - protein, 7 aa, synthetic construct
LASNLES

SEQ ID NO: 296 - protein, 9 aa, synthetic construct
QHSREVPWT

SEQ ID NO: 297 - protein, 118 aa, synthetic construct
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 298 - protein, 111 aa, synthetic construct
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 299 - protein, 448 aa, synthetic construct
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 300 - protein, 218 aa, synthetic construct
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 301 - protein, 5 aa, synthetic construct
NYGMN

SEQ ID NO: 302 - protein, 17 aa, synthetic construct
WINTYTGEPTYTDDFKG

SEQ ID NO: 303 - protein, 12 aa, synthetic construct
GGFGSSYWYFDV

SEQ ID NO: 304 - protein, 11 aa, synthetic construct
KASQDVSIAVA

SEQ ID NO: 305 - protein, 7 aa, synthetic construct
SASYRYT

SEQ ID NO: 306 - protein, 9 aa, synthetic construct
QQHYITPLT

SEQ ID NO: 307 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 308 - protein, 107 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 309 - protein, 5 aa, synthetic construct
TAGMQ

SEQ ID NO: 310 - protein, 17 aa, synthetic construct
WINTHSGVPKYAEDFKG

SEQ ID NO: 311 - protein, 12 aa, synthetic construct
SGFGSSYWYFDV

SEQ ID NO: 312 - protein, 11 aa, synthetic construct
KASQDVSTAVA

SEQ ID NO: 313 - protein, 7 aa, synthetic construct
SASYRYT

SEQ ID NO: 314 - protein, 9 aa, synthetic construct
QQHYITPLT

SEQ ID NO: 315 - protein, 121 aa, synthetic construct
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 316 - protein, 107 aa, synthetic construct
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIK

SEQ ID NO: 317 - protein, 5 aa, synthetic construct
SQNIY

SEQ ID NO: 318 - protein, 17 aa, synthetic construct
YIEPYNVVPMYNPKFKG

SEQ ID NO: 319 - protein, 8 aa, synthetic construct
SGSSNFDY

SEQ ID NO: 320 - protein, 12 aa, synthetic construct
SASSSISSHYLH

SEQ ID NO: 321 - protein, 7 aa, synthetic construct
RTSNLAS

SEQ ID NO: 322 - protein, 9 aa, synthetic construct
QQGSSLPLT

SEQ ID NO: 323 - protein, 117 aa, synthetic construct
EIQLVQSGAEVKKPGASVKVSCKASGYAFTSQNIYWVRQAPGQGLEWIGYIEPYNVVPMYNPKFKGRATLTVDKSTSTAYLELSSLRSEDTAVYYCARSGSSNFDYWGQGTLVTVSS

SEQ ID NO: 324 - protein, 108 aa, synthetic construct
DIQLTQSPSSLSASVGDRVTITCSASSSISSHYLHWYQQKPGKSPKLLIYRTSNLASGVPSRFSGSGSGTDYTLTISSLQPEDFATYYCQQGSSLPLTFGQGTKVEIK

SEQ ID NO: 325 - protein, 5 aa, synthetic construct
NYAMH

SEQ ID NO: 326 - protein, 17 aa, synthetic construct
LIWYDGSNKFYGDSVKG

SEQ ID NO: 327 - protein, 6 aa, synthetic construct
EGSGHY

SEQ ID NO: 328 - protein, 11 aa, synthetic construct
RASQGISSALA

SEQ ID NO: 329 - protein, 7 aa, synthetic construct
DASSLES

SEQ ID NO: 330 - protein, 9 aa, synthetic construct
QQFNSYPIT

SEQ ID NO: 331 - protein, 115 aa, synthetic construct
QVQLVESGGGVVQPGRSLRLSCAASGFTFSNYAMHWVRQAPGEGLEWVALIWYDGSNKFYGDSVKGRFTISRDNSKNTLYLQMNSLSAEDTAVYYCAREGSGHYWGQGTLVTVSS

SEQ ID NO: 332 - protein, 107 aa, synthetic construct
AIQLTQSPSSLSASVGDRVTITCRASQGISSALAWYQQKPGKVPKSLIYDASSLESGVPSRFSGSGSGTDFTLTISSLQPEDFATYYCQQFNSYPITFGQGTRLEIK

SEQ ID NO: 333 - protein, 5 aa, synthetic construct
NYAMS

SEQ ID NO: 334 - protein, 17 aa, synthetic construct
YISPGGDYIYYADSVKG

SEQ ID NO: 335 - protein, 12 aa, synthetic construct
DRRHYGSYAMDY

SEQ ID NO: 336 - protein, 16 aa, synthetic construct
RSSKSLLHSNLNTYLY

SEQ ID NO: 337 - protein, 7 aa, synthetic construct
RMSNLAS

SEQ ID NO: 338 - protein, 9 aa, synthetic construct
MQHLEYPFT

SEQ ID NO: 339 - protein, 121 aa, synthetic construct
QVQLVESGGGLVKPGGSLRLSCAASGFTFSNYAMSWIRQAPGKGLEWVSYISPGGDYIYYADSVKGRFTISRDNAKNSLYLQMNSLRAEDTAVYYCTTDRRHYGSYAMDYWGQGTLVTVSS

SEQ ID NO: 340 - protein, 112 aa, synthetic construct
DIVMTQSPLSLPVTPGEPASISCRSSKSLLHSNLNTYLYWFLQKPGQSPQILIYRMSNLASGVPDRFSGSGSGTAFTLKISRVEAEDVGVYYCMQHLEYPFTFGPGTKLEIK

SEQ ID NO: 341 - protein, 5 aa, synthetic construct
TYAFH

SEQ ID NO: 342 - protein, 17 aa, synthetic construct
GIVPIFGTLKYAQKFQD

SEQ ID NO: 343 - protein, 11 aa, synthetic construct
AIQLEGRPFDH

SEQ ID NO: 344 - protein, 11 aa, synthetic construct
RASQGITSYLA

SEQ ID NO: 345 - protein, 7 aa, synthetic construct
AASALQS

SEQ ID NO: 346 - protein, 10 aa, synthetic construct
QQVNRGAAIT

SEQ ID NO: 347 - protein, 120 aa, synthetic construct
QVQLVQSGAEVKKPGSSVRVSCRASGGSSTTYAFHWVRQAPGQGLEWMGGIVPIFGTLKYAQKFQDRVTLTADKSTGTAYMELNSLRLDDTAVYYCARAIQLEGRPFDHWGQGTQVTVSA

SEQ ID NO: 348 - protein, 108 aa, synthetic construct
DIQLTQSPSFLSASVGDRVTITCRASQGITSYLAWYQQKPGKAPKLLIYAASALQSGVPSRFSGRGSGTEFTLTISSLQPEDFATYYCQQVNRGAAITFGHGTRLDIK

SEQ ID NO: 349 - protein, 5 aa, synthetic construct
RYTMH

SEQ ID NO: 350 - protein, 17 aa, synthetic construct
VISFDGSNKYYVDSVKG

SEQ ID NO: 351 - protein, 10 aa, synthetic construct
EARGSYAFDI

SEQ ID NO: 352 - protein, 11 aa, synthetic construct
RASQSVSSYLA

SEQ ID NO: 353 - protein, 7 aa, synthetic construct
DASNRAT

SEQ ID NO: 354 - protein, 10 aa, synthetic construct
QQRSNWPPFT

SEQ ID NO: 355 - protein, 119 aa, synthetic construct
QVQLVESGGGVVQPGRSRRLSCAASGFTFSRYTMHWVRQAPGKGLEWVAVISFDGSNKYYVDSVKGRFTISRDNSENTLYLQVNILRAEDTAVYYCAREARGSYAFDIWGQGTMVTVSS

SEQ ID NO: 356 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGPGTKVDIK

SEQ ID NO: 357 - protein, 5 aa, synthetic construct
SFWMH

SEQ ID NO: 358 - protein, 17 aa, synthetic construct
YINPRSGYTEYNEIFRD

SEQ ID NO: 359 - protein, 9 aa, synthetic construct
FLGRGAMDY

SEQ ID NO: 360 - protein, 11 aa, synthetic construct
RASQDISNYLA

SEQ ID NO: 361 - protein, 7 aa, synthetic construct
YTSKIHS

SEQ ID NO: 362 - protein, 9 aa, synthetic construct
QQGNTFPYT

SEQ ID NO: 363 - protein, 118 aa, synthetic construct [sequence omitted]

SEQ ID NO: 364 - protein, 107 aa, synthetic construct [sequence omitted]

SEQ ID NO: 365 - protein, 7 aa, synthetic construct
TSSYYWG

SEQ ID NO: 366 - protein, 16 aa, synthetic construct
TIYYNGSTYYSPSLKS

SEQ ID NO: 367 - protein, 11 aa, synthetic construct
QGYDIKINIDV

SEQ ID NO: 368 - protein, 11 aa, synthetic construct
RASQSVSSYLA

SEQ ID NO: 369 - protein, 7 aa, synthetic construct
VASNRAT

SEQ ID NO: 370 - protein, 9 aa, synthetic construct
QQRSNWPLT

SEQ ID NO: 371 - protein, 121 aa, synthetic construct
QLQLQESGPGLVKPSETLSLTCTVSGGSISTSSYYWGWIRQPPGKGLEWIGTIYYNGSTYYSPSLKSRVSISVDTSKNQFSLKLSSVTAADTSVYYCARQGYDIKINIDVWGQGTTVTVSS

SEQ ID NO: 372 - protein, 107 aa, synthetic construct
EIVLTQSPATLSLSPGERATLSCRASQSVSSYLAWYQQKPGQAPRLLIYVASNRATGIPARFSGSGSGTDFTLTISSLEPEDFAVYYCQQRSNWPLTFGGGTKVEIK

SEQ ID NO: 373 - protein, 5 aa, synthetic construct
SSWMN

SEQ ID NO: 374 - protein, 17 aa, synthetic construct
RIYPGDGNTHYAQKFQG

SEQ ID NO: 375 - protein, 8 aa, synthetic construct
GYLDPMDY

SEQ ID NO: 376 - protein, 11 aa, synthetic construct
QASQGINNYLN

SEQ ID NO: 377 - protein, 7 aa, synthetic construct
YTSGLHA

SEQ ID NO: 378 - protein, 9 aa, synthetic construct
QQYSILPWT

SEQ ID NO: 379 - protein, 117 aa, synthetic construct
QVQLVQSGAEVKKPGSSVKVSCKASGYAFTSSWMNWVRQAPGQGLEWMGRIYPGDGNTHYAQKFQGRVTLTADKSTSTAYMELSSLRSEDTAVYYCGEGYLDPMDYWGQGTLVTVSS

SEQ ID NO: 380 - protein, 107 aa, synthetic construct
DIQMTQSPSSLSASVGDRVTITCQASQGINNYLNWYQQKPGKAPKLLIHYTSGLHAGVPSRFSGSGSGTDYTLTISSLEPEDVATYYCQQYSILPWTFGGGTKVEIK

SEQ ID NO: 381 - protein, 5 aa, synthetic construct
SYGMH

SEQ ID NO: 382 - protein, 17 aa, synthetic construct
VISYDGSNKYYADSVKG

SEQ ID NO: 383 - protein, 18 aa, synthetic construct
DMGWGSGWRPYYYYGMDV

SEQ ID NO: 384 - protein, 11 aa, synthetic construct
RTSQSISSYLN

SEQ ID NO: 385 - protein, 7 aa, synthetic construct
WASTRES

SEQ ID NO: 386 - protein, 9 aa, synthetic construct
QQSYDIPYT

SEQ ID NO: 387 - protein, 127 aa, synthetic construct
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDMGWGSGWRPYYYYGMDVWGQGTTVTVSS

SEQ ID NO: 388 - protein, 107 aa, synthetic construct
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIK

SEQ ID NO: 389 - protein, 5 aa, synthetic construct
NYWMS

SEQ ID NO: 390 - protein, 17 aa, synthetic construct
NIKQDGSEKFYADSVKG

SEQ ID NO: 391 - protein, 9 aa, synthetic construct
VGPSWEQDY

SEQ ID NO: 392 - protein, 14 aa, synthetic construct
TGSSSNIGSYYGVH

SEQ ID NO: 393 - protein, 7 aa, synthetic construct
SDTNRPS

SEQ ID NO: 394 - protein, 11 aa, synthetic construct
QSYDKGFGHRV

SEQ ID NO: 395 - protein, 118 aa, synthetic construct
EVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSA

SEQ ID NO: 396 - protein, 111 aa, synthetic construct
QSVLTQPPSVSGAPGQRVTISCTGSSSNIGSYYGVHWYQQLPGTAPKLLIYSDTNRPSGVPDRFSGSKSGTSASLAITGLQAEDEADYYCQSYDKGFGHRVFGGGTKLTVL

SEQ ID NO: 397 - protein, 5 aa, synthetic construct
SYAIS

SEQ ID NO: 398 - protein, 17 aa, synthetic construct
GIIPIFGTANYAQKFQG

SEQ ID NO: 399 - protein, 6 aa, synthetic construct
GLLWNY

SEQ ID NO: 400 - protein, 11 aa, synthetic construct
RASQSVSSNLA

SEQ ID NO: 401 - protein, 7 aa, synthetic construct
GASTTAS

SEQ ID NO: 402 - protein, 11 aa, synthetic construct
QQYNNWPPAYT

SEQ ID NO: 403 - protein, 115 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSS

SEQ ID NO: 404 - protein, 109 aa, synthetic construct
EIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIK

SEQ ID NO: 405 - protein, 5 aa, synthetic construct
NYGMN

SEQ ID NO: 406 - protein, 17 aa, synthetic construct
WINTYTGEPTYGEDFKG

SEQ ID NO: 407 - protein, 7 aa, synthetic construct
FGNYVDY

SEQ ID NO: 408 - protein, 16 aa, synthetic construct
RSSKNLLHSNGITYLY

SEQ ID NO: 409 - protein, 7 aa, synthetic construct
QMSNLAS

SEQ ID NO: 410 - protein, 9 aa, synthetic construct
AQNLEIPRT

SEQ ID NO: 411 - protein, 116 aa, synthetic construct
QVQLVQSGPEVKKPGASVKVSCKASGYTFTNYGMNWVRQAPGQGLEWMGWINTYTGEPTYGEDFKGRFAFSLDTSASTAYMELSSLRSEDTAVYFCARFGNYVDYWGQGSLVTVSS

SEQ ID NO: 412 - protein, 112 aa, synthetic construct
DIVMTQSPLSLPVTPGEPASISCRSSKNLLHSNGITYLYWYLQKPGQSPQLLIYQMSNLASGVPDRFSSSGSGTDFTLKISRVEAEDVGVYYCAQNLEIPRTFGQGTKVEIK

SEQ ID NO: 413 - protein, 5 aa, synthetic construct
KYGMN

SEQ ID NO: 414 - protein, 17 aa, synthetic construct
WINTYTEEPTYGDDFKG

SEQ ID NO: 415 - protein, 7 aa, synthetic construct
FGSAVDY

SEQ ID NO: 416 - protein, 16 aa, synthetic construct
RSSKSLLHSNGITYLY

SEQ ID NO: 417 - protein, 7 aa, synthetic construct
QMSNRAS

SEQ ID NO: 418 - protein, 9 aa, synthetic construct
AQNLELPRT

SEQ ID NO: 419 - protein, 116 aa, synthetic construct
QIQLVQSGPEVKKPGESVKISCKASGYTFTKYGMNWVKQAPGQGLKWMGWINTYTEEPTYGDDFKGRFTFTLDTSTSTAYLEISSLRSEDTATYFCARFGSAVDYWGQGTLVTVSS

SEQ ID NO: 420 - protein, 113 aa, synthetic construct
DIVMTQSALSNPVTLGESGSISCRSSKSLLHSNGITYLYWYLQKPGQSPQLLIYQMSNRASGVPDRFSSSGSGTDFTLKISRVEAEDVGVYYCAQNLELPRTFGQGTKLEMKR

SEQ ID NO: 421 - protein, 5 aa, synthetic construct
DYSMH

SEQ ID NO: 422 - protein, 17 aa, synthetic construct
WINTETGEPTYADDFKG

SEQ ID NO: 423 - protein, 4 aa, synthetic construct
TAVY

SEQ ID NO: 424 - protein, 11 aa, synthetic construct
RASQEISVSLS

SEQ ID NO: 425 - protein, 7 aa, synthetic construct
ATSTLDS

SEQ ID NO: 426 - protein, 9 aa, synthetic construct
LQYASYPWT

SEQ ID NO: 427 - protein, 113 aa, synthetic construct
QVKLQESGPELKKPGETVKISCKASGYTFTDYSMHWVKQAPGKGLKWMGWINTETGEPTYADDFKGRFAFSLETSASTAYLQINNLKNEDTATYFCARTAVYWGQGTTVTVSS

SEQ ID NO: 428 - protein, 108 aa, synthetic construct
DIQMTQSPSSLSASLGERVSLTCRASQEISVSLSWLQQEPDGTIKRLIYATSTLDSGVPKRFSGSRSGSDYSLTISSLESEDFVDYYCLQYASYPWTFGGGTKLEIKR

SEQ ID NO: 429 - protein, 5 aa, synthetic construct
NYGMN

SEQ ID NO: 430 - protein, 17 aa, synthetic construct
WINTYSGEPRYADDFKG

SEQ ID NO: 431 - protein, 9 aa, synthetic construct
DYGRWYFDV

SEQ ID NO: 432 - protein, 10 aa, synthetic construct
RASSSVSHMH

SEQ ID NO: 433 - protein, 7 aa, synthetic construct
ATSNLAS

SEQ ID NO: 434 - protein, 9 aa, synthetic construct
QQWSSTPRT

SEQ ID NO: 435 - protein, 118 aa, synthetic construct
QIQLVQSGSELKKPGASVKVSCKASGYTFTNYGMNWVRQAPGQDLKWMGWINTYSGEPRYADDFKGRFVFSLDKSVNTAYLQISSLKAEDTAVYYCARDYGRWYFDVWGQGTTVTVSS

SEQ ID NO: 436 - protein, 107 aa, synthetic construct
QIVLSQSPATLSLSPGERATMSCRASSSVSHMHWYQQKPGQAPRPWIYATSNLASGVPARFSGSGSGTDYTLTISSLEPEDFAVYYCQQWSSTPRTFGGGTKVEIKR

SEQ ID NO: 437 - protein, 5 aa, synthetic construct
RYWMS

SEQ ID NO: 438 - protein, 17 aa, synthetic construct
EINPDSSTINYAPSLKD

SEQ ID NO: 439 - protein, 10 aa, synthetic construct
PDGNYWYFDV

SEQ ID NO: 440 - protein, 11 aa, synthetic construct
KASQDVGIAVA

SEQ ID NO: 441 - protein, 7 aa, synthetic construct
WASTRHT

SEQ ID NO: 442 - protein, 9 aa, synthetic construct
QQYSSYPYT

SEQ ID NO: 443 - protein, 119 aa, synthetic construct
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 444 - protein, 108 aa, synthetic construct
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKR

SEQ ID NO: 445 - protein, 5 aa, synthetic construct
SFAMS

SEQ ID NO: 446 - protein, 17 aa, synthetic construct
AISGSGGGTYYADSVKG

SEQ ID NO: 447 - protein, 13 aa, synthetic construct
DKILWFGEPVFDY

SEQ ID NO: 448 - protein, 11 aa, synthetic construct
RASQSVSSYLA

SEQ ID NO: 449 - protein, 7 aa, synthetic construct
DASNRAT

SEQ ID NO: 450 - protein, 9 aa, synthetic construct
QQRSNWPPT

SEQ ID NO: 451 - protein, 122 aa, synthetic construct [sequence omitted]

SEQ ID NO: 452 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKR

SEQ ID NO: 453 - protein, 5 aa, synthetic construct
SYRMH

SEQ ID NO: 454 - protein, 17 aa, synthetic construct
YINPSTGYTEYNQKFKD

SEQ ID NO: 455 - protein, 7 aa, synthetic construct
GGGVFDY

SEQ ID NO: 456 - protein, 10 aa, synthetic construct
SASSSISYMH

SEQ ID NO: 457 - protein, 7 aa, synthetic construct
TTSNLAS

SEQ ID NO: 458 - protein, 9 aa, synthetic construct
HQRSTYPLT

SEQ ID NO: 459 - protein, 116 aa, synthetic construct
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSS

SEQ ID NO: 460 - protein, 106 aa, synthetic construct
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVK

SEQ ID NO: 461 - protein, 5 aa, synthetic construct
SYWMH

SEQ ID NO: 462 - protein, 17 aa, synthetic construct
EIIPINGHTNYNEKFKS

SEQ ID NO: 463 - protein, 14 aa, synthetic construct
GGYYYYGSRDYFDY

SEQ ID NO: 464 - protein, 15 aa, synthetic construct
KASQSVDYDGDSYMN

SEQ ID NO: 465 - protein, 7 aa, synthetic construct
AASDLES

SEQ ID NO: 466 - protein, 9 aa, synthetic construct
QQSHEDPFT

SEQ ID NO: 467 - protein, 123 aa, synthetic construct
QVQLQQPGAELVKPGASVKLSCKASGYTFTSYWMHWVKQRPGQGLEWIGEIIPINGHTNYNEKFKSKATLTLDKSSSTAYMQLSSLASEDSAVYYCARGGYYYYGSRDYFDYWGQGTTLTVSS

SEQ ID NO: 468 - protein, 111 aa, synthetic construct
DIVLTQSPASLAVSLGQRATISCKASQSVDYDGDSYMNWYQQIPGQPPKLLIYAASDLESGIPARFSGSGSGTDFTLNIHPVEEEDAATYYCQQSHEDPFTFGGGTKLEIK

SEQ ID NO: 469 - protein, 5 aa, synthetic construct
SYWMH

SEQ ID NO: 470 - protein, 17 aa, synthetic construct
EIIPIFGHTNYNEKFKS

SEQ ID NO: 471 - protein, 14 aa, synthetic construct
GGYYYYPRQGFLDY

SEQ ID NO: 472 - protein, 15 aa, synthetic construct
KASQSVDYSGDSYMN

SEQ ID NO: 473 - protein, 7 aa, synthetic construct
AASDLES

SEQ ID NO: 474 - protein, 9 aa, synthetic construct
QQSHEDPFT

SEQ ID NO: 475 - protein, 123 aa, synthetic construct
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSS

SEQ ID NO: 476 - protein, 111 aa, synthetic construct
DIVMTQSPDSLAVSLGERATISCKASQSVDYSGDSYMNWYQQKPGQPPKLLIYAASDLESGIPARFSGSGSGTDFTLTISSLEPEDFATYYCQQSHEDPFTFGQGTKLEIK

SEQ ID NO: 477 - protein, 5 aa, synthetic construct
SYGMN

SEQ ID NO: 478 - protein, 17 aa, synthetic construct
YISSSSSTIYYADSVKG

SEQ ID NO: 479 - protein, 6 aa, synthetic construct
GPGMDV

SEQ ID NO: 480 - protein, 17 aa, synthetic construct
KSSQSVLYSSNNKNYLA

SEQ ID NO: 481 - protein, 7 aa, synthetic construct
WASTRES

SEQ ID NO: 482 - protein, 10 aa, synthetic construct
QQYYSTPQLT

SEQ ID NO: 483 - protein, 114 aa, synthetic construct
QVQLQQSGGGVVQPGRSLGLSCAASGFTFSSYGMNWVRQAPGKGLEWVSYISSSSSTIYYADSVKGRFTISRDNSKNTLYLQMNSLRAEDTAVYYCARGPGMDVWGQGTTVTVS

SEQ ID NO: 484 - protein, 114 aa, synthetic construct
DIVLTQSPDSLAVSLGERATINCKSSQSVLYSSNNKNYLAWYQQKPGQPPKLLIYWASTRESGVPDRFSGSGSGTDFTPAISSLQAEDVAVYYCQQYYSTPQLTFGGGTKVDIK

SEQ ID NO: 485 - protein, 7 aa, synthetic construct
TSGMGVG

SEQ ID NO: 486 - protein, 16 aa, synthetic construct
HIWWDDDKRYNPALKS

SEQ ID NO: 487 - protein, 10 aa, synthetic construct
MELWSYYFDY

SEQ ID NO: 488 - protein, 10 aa, synthetic construct
SASSSVSYMH

SEQ ID NO: 489 - protein, 7 aa, synthetic construct
DTSKLAS

SEQ ID NO: 490 - protein, 9 aa, synthetic construct
FQGSVYPFT

SEQ ID NO: 491 - protein, 120 aa, synthetic construct
QVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSS

SEQ ID NO: 492 - protein, 107 aa, synthetic construct
EIVLTQSPATLSLSPGERATLSCSASSSVSYMHWYQQKPGQAPRLLIYDTSKLASGIPARFSGSGSGTDFTLTISSLEPEDVAVYYCFQGSVYPFTFGQGTKLEIKR

SEQ ID NO: 493 - protein, 450 aa, synthetic construct
QVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 494 - protein, 213 aa, synthetic construct
EIVLTQSPATLSLSPGERATLSCSASSSVSYMHWYQQKPGQAPRLLIYDTSKLASGIPARFSGSGSGTDFTLTISSLEPEDVAVYYCFQGSVYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 495 - protein, 5 aa, synthetic construct
NYWIE

SEQ ID NO: 496 - protein, 17 aa, synthetic construct
EILPGTGRTIYNEKFKG

SEQ ID NO: 497 - protein, 13 aa, synthetic construct
RDYYGNFYYAMDY

SEQ ID NO: 498 - protein, 11 aa, synthetic construct
SASQGINNYLN

SEQ ID NO: 499 - protein, 7 aa, synthetic construct
YTSTLQS

SEQ ID NO: 500 - protein, 9 aa, synthetic construct
QQYSKLPRT

SEQ ID NO: 501 - protein, 122 aa, synthetic construct
QVQLQQSGSELMMPGASVKISCKATGYTFSNYWIEWVKQRPGHGLEWIGEILPGTGRTIYNEKFKGKATFTADISSNTVQMQLSSLTSEDSAVYYCARRDYYGNFYYAMDYWGQGTSVTVSS

SEQ ID NO: 502 - protein, 107 aa, synthetic construct
DIQMTQSTSSLSASLGDRVTISCSASQGINNYLNWYQQKPDGTVELLIYYTSTLQSGVPSRFSGSGSGTDYSLTISNLEPEDIGTYYCQQYSKLPRTFGGGTKLEIK

SEQ ID NO: 503 - protein, 7 aa, synthetic construct
TYGMGVG

SEQ ID NO: 504 - protein, 16 aa, synthetic construct
NIWWSEDKHYSPSLKS

SEQ ID NO: 505 - protein, 11 aa, synthetic construct
IDYGNDYAFTY

SEQ ID NO: 506 - protein, 16 aa, synthetic construct
RSSKSLLHSNGITYLY

SEQ ID NO: 507 - protein, 7 aa, synthetic construct
QMSNLAS

SEQ ID NO: 508 - protein, 9 aa, synthetic construct
AQNLELPYT

SEQ ID NO: 509 - protein, 121 aa, synthetic construct
QITLKESGPTLVKPTQTLTLTCTFSGFSLSTYGMGVGWIRQPPGKALEWLANIWWSEDKHYSPSLKSRLTITKDTSKNQVVLTITNVDPVDTATYYCVQIDYGNDYAFTYWGQGTLVTVSS

SEQ ID NO: 510 - protein, 112 aa, synthetic construct
DIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIK

SEQ ID NO: 511 - protein, 5 aa, synthetic construct
SFGMH

SEQ ID NO: 512 - protein, 17 aa, synthetic construct
YISSGSFTIYYADSVKG

SEQ ID NO: 513 - protein, 9 aa, synthetic construct
MRKGYAMDY

SEQ ID NO: 514 - protein, 16 aa, synthetic construct
RSSQIIIHSDGNTYLE

SEQ ID NO: 515 - protein, 7 aa, synthetic construct
KVSNRFS

SEQ ID NO: 516 - protein, 9 aa, synthetic construct
FQGSHVPHT

SEQ ID NO: 517 - protein, 118 aa, synthetic construct
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSS

SEQ ID NO: 518 - protein, 112 aa, synthetic construct
DVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIK

SEQ ID NO: 519 - protein, 5 aa, synthetic construct
NYGVN

SEQ ID NO: 520 - protein, 17 aa, synthetic construct
WINPNTGEPTFDDDFKG

SEQ ID NO: 521 - protein, 11 aa, synthetic construct
SRGKNEAWFAY

SEQ ID NO: 522 - protein, 16 aa, synthetic construct
RSSQSLVHRNGNTYLH

SEQ ID NO: 523 - protein, 7 aa, synthetic construct
TVSNRFS

SEQ ID NO: 524 - protein, 9 aa, synthetic construct
SQSSHVPPT

SEQ ID NO: 525 - protein, 120 aa, synthetic construct
QVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 526 - protein, 112 aa, synthetic construct
DIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIK

SEQ ID NO: 527 - protein, 5 aa, synthetic construct
TYWMS

SEQ ID NO: 528 - protein, 17 aa, synthetic construct
EIHPDSSTINYAPSLKD

SEQ ID NO: 529 - protein, 10 aa, synthetic construct
LYFGFPWFAY

SEQ ID NO: 530 - protein, 11 aa, synthetic construct
KASQDVGTSVA

SEQ ID NO: 531 - protein, 7 aa, synthetic construct
WTSTRHT

SEQ ID NO: 532 - protein, 8 aa, synthetic construct
QQYSLYRS

SEQ ID NO: 533 - protein, 119 aa, synthetic construct
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSS

SEQ ID NO: 534 - protein, 106 aa, synthetic construct
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIK

SEQ ID NO: 535 - protein, 5 aa, synthetic construct
YYGMN

SEQ ID NO: 536 - protein, 17 aa, synthetic construct
WIDTTTGEPTYAQKFQG

SEQ ID NO: 537 - protein, 10 aa, synthetic construct
RGPYNWYFDV

SEQ ID NO: 538 - protein, 16 aa, synthetic construct
RSSKSLLHSNGNTYLY

SEQ ID NO: 539 - protein, 7 aa, synthetic construct
RMSNLVS

SEQ ID NO: 540 - protein, 9 aa, synthetic construct
LQHLEYPFT

SEQ ID NO: 541 - protein, 119 aa, synthetic construct
QVQLVQSGAEVKKPGETVKISCKASDYTFTYYGMNWVKQAPGQGLKWMGWIDTTTGEPTYAQKFQGRIAFSLETSASTAYLQIKSLKSEDTATYFCARRGPYNWYFDVWGQGTTVTVSS

SEQ ID NO: 542 - protein, 112 aa, synthetic construct
DIVMTQSPLSVPVTPGEPVSISCRSSKSLLHSNGNTYLYWFLQRPGQSPQLLIYRMSNLVSGVPDRFSGSGSGTAFTLRISRVEAEDVGVYYCLQHLEYPFTFGPGTKLELK

SEQ ID NO: 543 - protein, 5 aa, synthetic construct
NYGMN

SEQ ID NO: 544 - protein, 17 aa, synthetic construct
WINTYTGEPTYADDFKG

SEQ ID NO: 545 - protein, 9 aa, synthetic construct
IGDSSPSDY

SEQ ID NO: 546 - protein, 11 aa, synthetic construct
KASQSVSNDVV

SEQ ID NO: 547 - protein, 7 aa, synthetic construct
YASNRYT

SEQ ID NO: 548 - protein, 9 aa, synthetic construct
QQDYTSPWT

SEQ ID NO: 549 - protein, 118 aa, synthetic construct
QVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSS

SEQ ID NO: 550 - protein, 107 aa, synthetic construct
EIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIK

SEQ ID NO: 551 - protein, 5 aa, synthetic construct
SYWIE

SEQ ID NO: 552 - protein, 17 aa, synthetic construct
EILPGSGNTYYNERFKD

SEQ ID NO: 553 - protein, 14 aa, synthetic construct
RAAAYYSNPEWFAY

SEQ ID NO: 554 - protein, 12 aa, synthetic construct
TASSSVNSFYLH

SEQ ID NO: 555 - protein, 7 aa, synthetic construct
STSNLAS

SEQ ID NO: 556 - protein, 9 aa, synthetic construct
HQYHRSPYT

SEQ ID NO: 557 - protein, 123 aa, synthetic construct
QVQLVQSGAEVKKPGSSVKVSCKASGGTFSSYWIEWVRQAPGQGLEWMGEILPGSGNTYYNERFKDRVTITADESTSTAYMELSSLRSEDTAVYYCARRAAAYYSNPEWFAYWGQGTLVTVSS

SEQ ID NO: 558 - protein, 108 aa, synthetic construct
EIVLTQSPATLSLSPGERATLSCTASSSVNSFYLHWYQQKPGLAPRLLIYSTSNLASGIPDRFSGSGSGTDFTLTISRLEPEDFAVYYCHQYHRSPYTFGQGTKLEIK

SEQ ID NO: 559 - protein, 5 aa, synthetic construct
SYWMQ

SEQ ID NO: 560 - protein, 17 aa, synthetic construct
TIYPGDGDTTYTQKFQG

SEQ ID NO: 561 - protein, 10 aa, synthetic construct
YDAPGYAMDY

SEQ ID NO: 562 - protein, 11 aa, synthetic construct
RASQDINNYLA

SEQ ID NO: 563 - protein, 7 aa, synthetic construct
YTSTLHP

SEQ ID NO: 564 - protein, 9 aa, synthetic construct
LQYDNLLYT

SEQ ID NO: 565 - protein, 119 aa, synthetic construct
QVQLVQSGAEVAKPGASVKLSCKASGYTFTSYWMQWVKQRPGQGLECIGTIYPGDGDTTYTQKFQGKATLTADKSSSTAYMQLSSLRSEDSAVYYCARYDAPGYAMDYWGQGTLVTVSS

SEQ ID NO: 566 - protein, 107 aa, synthetic construct
DIQMTQSPSSLSASVGDRVTITCRASQDINNYLAWYQHKPGKGPKLLIHYTSTLHPGIPSRFSGSGSGRDYSFSISSLEPEDIATYYCLQYDNLLYTFGQGTKLEIK

SEQ ID NO: 567 - protein, 6 aa, synthetic construct
RDFAWN

SEQ ID NO: 568 - protein, 16 aa, synthetic construct
YISYNGNTRYQPSLKS

SEQ ID NO: 569 - protein, 7 aa, synthetic construct
ASRGFPY

SEQ ID NO: 570 - protein, 11 aa, synthetic construct
HSSQDINSNIG

SEQ ID NO: 571 - protein, 7 aa, synthetic construct
HGTNLDD

SEQ ID NO: 572 - protein, 9 aa, synthetic construct
VQYAQFPWT

SEQ ID NO: 573 - protein, 116 aa, synthetic construct
EVQLQESGPGLVKPSQTLSLTCTVSGYSISRDFAWNWIRQPPGKGLEWMGYISYNGNTRYQPSLKSRITISRDTSKNQFFLKLNSVTAADTATYYCVTASRGFPYWGQGTLVTVSS

SEQ ID NO: 574 - protein, 107 aa, synthetic construct
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIK

SEQ ID NO: 575 - protein, 5 aa, synthetic construct
NYGVH

SEQ ID NO: 576 - protein, 16 aa, synthetic construct
VIWSGGNTDYNTPFTS

SEQ ID NO: 577 - protein, 11 aa, synthetic construct
ALTYYDYEFAY

SEQ ID NO: 578 - protein, 11 aa, synthetic construct
RASQSIGTNIH

SEQ ID NO: 579 - protein, 7 aa, synthetic construct
YASESIS

SEQ ID NO: 580 - protein, 9 aa, synthetic construct
QQNNNWPTT

SEQ ID NO: 581 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 582 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 583 - protein, 5 aa, synthetic construct
GYFMN

SEQ ID NO: 584 - protein, 17 aa, synthetic construct
RIHPYDGDTFYNQKFQG

SEQ ID NO: 585 - protein, 9 aa, synthetic construct
YDGSRAMDY

SEQ ID NO: 586 - protein, 15 aa, synthetic construct
KASQSVSFAGTSLMH

SEQ ID NO: 587 - protein, 7 aa, synthetic construct
RASNLEA

SEQ ID NO: 588 - protein, 9 aa, synthetic construct
QQSREYPYT

SEQ ID NO: 589 - protein, 118 aa, synthetic construct
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSS

SEQ ID NO: 590 - protein, 111 aa, synthetic construct
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIK

SEQ ID NO: 591 - protein, 5 aa, synthetic construct
GYGLS

SEQ ID NO: 592 - protein, 17 aa, synthetic construct
MISSGGSYTYYADSVKG

SEQ ID NO: 593 - protein, 10 aa, synthetic construct
HGDDPAWFAY

SEQ ID NO: 594 - protein, 12 aa, synthetic construct
SVSSSISSNNLH

SEQ ID NO: 595 - protein, 7 aa, synthetic construct
GTSNLAS

SEQ ID NO: 596 - protein, 11 aa, synthetic construct
QQWSSYPYMYT

SEQ ID NO: 597 - protein, 119 aa, synthetic construct
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSS

SEQ ID NO: 598 - protein, 110 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIK

SEQ ID NO: 599 - protein, 5 aa, synthetic construct
NYWMN

SEQ ID NO: 600 - protein, 19 aa, synthetic construct
EIRLKSNNYTTHYAESVKG

SEQ ID NO: 601 - protein, 6 aa, synthetic construct
HYYFDY

SEQ ID NO: 602 - protein, 16 aa, synthetic construct
RSSKSLLHSNGITYFF

SEQ ID NO: 603 - protein, 7 aa, synthetic construct
QMSNLAS

SEQ ID NO: 604 - protein, 9 aa, synthetic construct
AQNLELPPT

SEQ ID NO: 605 - protein, 117 aa, synthetic construct
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSS

SEQ ID NO: 606 - protein, 112 aa, synthetic construct
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIK

SEQ ID NO: 607 - protein, 5 aa, synthetic construct
SYWIG

SEQ ID NO: 608 - protein, 17 aa, synthetic construct
IIDPGDSRTRYSPSFQG

SEQ ID NO: 609 - protein, 11 aa, synthetic construct
GQLYGGTYMDG

SEQ ID NO: 610 - protein, 14 aa, synthetic construct
TGTSSDIGGYNSVS

SEQ ID NO: 611 - protein, 7 aa, synthetic construct
GVNNRPS

SEQ ID NO: 612 - protein, 11 aa, synthetic construct
SSYDIESATPV

SEQ ID NO: 613 - protein, 120 aa, synthetic construct
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 614 - protein, 111 aa, synthetic construct
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVL

SEQ ID NO: 615 - protein, 5 aa, synthetic construct
AYNIH

SEQ ID NO: 616 - protein, 17 aa, synthetic construct
SFDPYDGGSSYNQKFKD

SEQ ID NO: 617 - protein, 7 aa, synthetic construct
GWYYFDY

SEQ ID NO: 618 - protein, 11 aa, synthetic construct
RASKSISKYLA

SEQ ID NO: 619 - protein, 7 aa, synthetic construct
SGSTLQS

SEQ ID NO: 620 - protein, 9 aa, synthetic construct
QQHDESPYT

SEQ ID NO: 621 - protein, 116 aa, synthetic construct
QVQLQESGPGLVKPSQTLSLTCTVSGYAFTAYNIHWVRQAPGQGLEWMGSFDPYDGGSSYNQKFKDRLTISKDTSKNQVVLTMTNMDPVDTATYYCARGWYYFDYWGHGTLVTVSS

SEQ ID NO: 622 - protein, 107 aa, synthetic construct
DIVMTQTPLSLPVTPGEPASISCRASKSISKYLAWYQQKPGQAPRLLIYSGSTLQSGIPPRFSGSGYGTDFTLTINNIESEDAAYYFCQQHDESPYTFGEGTKVEIK

SEQ ID NO: 623 - protein, 5 aa, synthetic construct
SFGMH

SEQ ID NO: 624 - protein, 17 aa, synthetic construct
YISSDSSAIYYADTVKG

SEQ ID NO: 625 - protein, 13 aa, synthetic construct
GRENIYYGSRLDY

SEQ ID NO: 626 - protein, 11 aa, synthetic construct
KASQNVDTNVA

SEQ ID NO: 627 - protein, 7 aa, synthetic construct
SASYRYS

SEQ ID NO: 628 - protein, 9 aa, synthetic construct
QQYNNYPFT

SEQ ID NO: 629 - protein, 122 aa, synthetic construct
DVQLVESGGGLVQPGGSRKLSCAASGFTFSSFGMHWVRQAPEKGLEWVAYISSDSSAIYYADTVKGRFTISRDNPKNTLFLQMTSLRSEDTAMYYCGRGRENIYYGSRLDYWGQGTTLTVSS

SEQ ID NO: 630 - protein, 107 aa, synthetic construct
DIAMTQSQKFMSTSVGDRVSVTCKASQNVDTNVAWYQQKPGQSPKALIYSASYRYSGVPDRFTGSGSGTDFTLTINNVQSEDLAEYFCQQYNNYPFTFGSGTKLEIK

SEQ ID NO: 631 - protein, 5 aa, synthetic construct
SYGMS

SEQ ID NO: 632 - protein, 17 aa, synthetic construct
TINSGGSNTYYPDSLKG

SEQ ID NO: 633 - protein, 8 aa, synthetic construct
HDGGAMDY

SEQ ID NO: 634 - protein, 11 aa, synthetic construct
RASESIYSYLA

SEQ ID NO: 635 - protein, 7 aa, synthetic construct
NTKTLPE

SEQ ID NO: 636 - protein, 10 aa, synthetic construct
QHHYGTPPWT

SEQ ID NO: 637 - protein, 117 aa, synthetic construct
EVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSS

SEQ ID NO: 638 - protein, 108 aa, synthetic construct
DIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIK

SEQ ID NO: 639 - protein, 5 aa, synthetic construct
SFGMH

SEQ ID NO: 640 - protein, 17 aa, synthetic construct
YISSGSGTIYYADTVKG

SEQ ID NO: 641 - protein, 10 aa, synthetic construct
HGYRYEGFDY

SEQ ID NO: 642 - protein, 11 aa, synthetic construct
KASQNVDTNVA

SEQ ID NO: 643 - protein, 7 aa, synthetic construct
SASYRYS

SEQ ID NO: 644 - protein, 9 aa, synthetic construct
QQYNNYPFT

SEQ ID NO: 645 - protein, 119 aa, synthetic construct
EVQLVESGGGLVQPGGSLRLSCAASGFTFSSFGMHWVRQAPGKGLEWVAYISSGSGTIYYADTVKGRFTISRDNAKNSLYLQMNSLRAEDTAVYYCARHGYRYEGFDYWGQGTTVTVSS

SEQ ID NO: 646 - protein, 107 aa, synthetic construct
DIQMTQSPSFLSASVGDRVTITCKASQNVDTNVAWYQQKPGKAPKALIYSASYRYSGVPSRFSGSGSGTDFTLTISSLQPEDFAEYFCQQYNNYPFTFGQGTKLEIK

SEQ ID NO: 647 - protein, 5 aa, synthetic construct
NYVMH

SEQ ID NO: 648 - protein, 17 aa, synthetic construct
YINPYNDDVKYNEKFKG

SEQ ID NO: 649 - protein, 13 aa, synthetic construct
WGYYGSPLYYFDY

SEQ ID NO: 650 - protein, 10 aa, synthetic construct
RASSRLIYMH

SEQ ID NO: 651 - protein, 7 aa, synthetic construct
ATSNLAS

SEQ ID NO: 652 - protein, 9 aa, synthetic construct
QQWNSNPPT

SEQ ID NO: 653 - protein, 122 aa, synthetic construct
EVQLQQSGPELVKPGASVKMSCKASGYTFTNYVMHWVKQKPGQGLEWIGYINPYNDDVKYNEKFKGKATQTSDKSSSTAYMELSSLTSEDSAVYYCARWGYYGSPLYYFDYWGQGTTLTVSS

SEQ ID NO: 654 - protein, 106 aa, synthetic construct
QIVLSQSPTILSASPGEKVTMTCRASSRLIYMHWYQQKPGSSPKPWIYATSNLASGVPARFSGSGSGTSYSLTISRVEAEDAATYYCQQWNSNPPTFGTGTKLELK

SEQ ID NO: 655 - protein, 5 aa, synthetic construct
NYVMH

SEQ ID NO: 656 - protein, 17 aa, synthetic construct
YINPYNDDVKYNEKFKG

SEQ ID NO: 657 - protein, 13 aa, synthetic construct
WGYYGSPLYYFDY

SEQ ID NO: 658 - protein, 10 aa, synthetic construct
RASSRLIYMH

SEQ ID NO: 659 - protein, 7 aa, synthetic construct
ATSNLAS

SEQ ID NO: 660 - protein, 9 aa, synthetic construct
QQWNSNPPT

SEQ ID NO: 661 - protein, 122 aa, synthetic construct
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSS

SEQ ID NO: 662 - protein, 106 aa, synthetic construct
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIK

SEQ ID NO: 663 - protein, 5 aa, synthetic construct
SYTIH

SEQ ID NO: 664 - protein, 17 aa, synthetic construct
YINPNSRNTDYAQKFQG

SEQ ID NO: 665 - protein, 12 aa, synthetic construct
YSGSTPYWYFDV

SEQ ID NO: 666 - protein, 10 aa, synthetic construct
RASSSVSYMN

SEQ ID NO: 667 - protein, 7 aa, synthetic construct
ATSNLAS

SEQ ID NO: 668 - protein, 9 aa, synthetic construct
QQWSSNPLT

SEQ ID NO: 669 - protein, 121 aa, synthetic construct
EVQLVQSGAEVKKPGSSVKVSCKASGYSFTSYTIHWVRQAPGQGLEWMGYINPNSRNTDYAQKFQGRVTLTADKSTSTAYMELSSLRSEDTAVYYCARYSGSTPYWYFDVWGQGTTVTVSS

SEQ ID NO: 670 - protein, 106 aa, synthetic construct
DIQLTQSPSFLSASVGDRVTITCRASSSVSYMNWYQQKPGKSPKPWIYATSNLASGVPSRFSVSVSGTEHTLTISSLQPEDFATYYCQQWSSNPLTFGQGTKLEIK

SEQ ID NO: 671 - protein, 5 aa, synthetic construct
SYWMH

SEQ ID NO: 672 - protein, 17 aa, synthetic construct
LIHPDSGSTNYNEMFKN

SEQ ID NO: 673 - protein, 8 aa, synthetic construct
GGRLYFDY

SEQ ID NO: 674 - protein, 16 aa, synthetic construct
RSSQSLVHSNGDTYLR

SEQ ID NO: 675 - protein, 7 aa, synthetic construct
KVSNRFS

SEQ ID NO: 676 - protein, 9 aa, synthetic construct
SQSTHVPYT

SEQ ID NO: 677 - protein, 117 aa, synthetic construct
EVQLVQSGAEVKKPGSSVKVSCKASGYTFSSYWMHWVRQAPGQGLEWIGLIHPDSGSTNYNEMFKNRATLTVDRSTSTAYVELSSLRSEDTAVYFCAGGGRLYFDYWGQGTTVTVSS

SEQ ID NO: 678 - protein, 112 aa, synthetic construct
DVVMTQSPLSLPVTPGEPASISCRSSQSLVHSNGDTYLRWYLQKPGQSPQLLIYKVSNRFSGVPDRFSGSGSGTDFTLKISRVEAEDVGVYYCSQSTHVPYTFGGGTKVEIK

SEQ ID NO: 679 - protein, 5 aa, synthetic construct
SYWMH

SEQ ID NO: 680 - protein, 17 aa, synthetic construct
LIHPESGSTNYNEMFKN

SEQ ID NO: 681 - protein, 8 aa, synthetic construct
GGRLYFDY

SEQ ID NO: 682 - protein, 16 aa, synthetic construct
RSSQSLVHSNQDTYLR

SEQ ID NO: 683 - protein, 7 aa, synthetic construct
KVSNRFS

SEQ ID NO: 684 - protein, 9 aa, synthetic construct
SQSTHVPYT

SEQ ID NO: 685 - protein, 117 aa, synthetic construct
EVQLVQSGAEVKKPGSSVKVSCKASGYTFSSYWMHWVRQAPGQGLEWIGLIHPESGSTNYNEMFKNRATLTVDRSTSTAYMELSSLRSEDTAVYYCAGGGRLYFDYWGQGTTVTVSS

SEQ ID NO: 686 - protein, 112 aa, synthetic construct
DIVMTQSPLSLPVTPGEPASISCRSSQSLVHSNQDTYLRWYLQKPGQSPQLLIYKVSNRFSGVPDRFSGSGSGTDFTLKISRVEAEDVGVYYCSQSTHVPYTFGGGTKVEIK

SEQ ID NO: 687 - protein, 6 aa, synthetic construct
SGYSWH

SEQ ID NO: 688 - protein, 16 aa, synthetic construct
YIHSSGSTNYNPSLKS

SEQ ID NO: 689 - protein, 7 aa, synthetic construct
YDDYFEY

SEQ ID NO: 690 - protein, 12 aa, synthetic construct
KASQNVGFNVAW

SEQ ID NO: 691 - protein, 7 aa, synthetic construct
SASYRYS

SEQ ID NO: 692 - protein, 9 aa, synthetic construct
QQYNWYPFT

SEQ ID NO: 693 - protein, 116 aa, synthetic construct
EVQLQESGPGLVKPSETLSLTCAVTGYSITSGYSWHWIRQFPGNGLEWMGYIHSSGSTNYNPSLKSRISISRDTSKNQFFLKLSSVTAADTAVYYCAGYDDYFEYWGQGTTVTVSS

SEQ ID NO: 694 - protein, 107 aa, synthetic construct
DIQMTQSPSSLSASVGDRVTITCKASQNVGFNVAWYQQKPGKSPKALIYSASYRYSGVPSRFSGSGSGTDFTLTISSLQPEDFAEYFCQQYNWYPFTFGQGTKLEIK

SEQ ID NO: 695 - protein, 5 aa, synthetic construct
NYDIN

SEQ ID NO: 696 - protein, 17 aa, synthetic construct
WIFPGDDSTQYNEKFKG

SEQ ID NO: 697 - protein, 9 aa, synthetic construct
QTTGTWFAY

SEQ ID NO: 698 - protein, 11 aa, synthetic construct
RASQSISDYLY

SEQ ID NO: 699 - protein, 7 aa, synthetic construct
YASQSIS

SEQ ID NO: 700 - protein, 9 aa, synthetic construct
QNGHSFPLT

SEQ ID NO: 701 - protein, 118 aa, synthetic construct
QVQLVQSGAEVVKPGASVKLSCKTSGYTFTNYDINWVRQRPGQGLEWIGWIFPGDDSTQYNEKFKGKATLTTDTSTSTAYMELSSLRSEDTAVYFCARQTTGTWFAYWGQGTLVTVSS

SEQ ID NO: 702 - protein, 107 aa, synthetic construct
EIVMTQSPATLSVSPGERVTLSCRASQSISDYLYWYQQKSHESPRLLIKYASQSISGIPARFSGSGSGSEFTLTINSVEPEDVGVYYCQNGHSFPLTFGQGTKLELK

SEQ ID NO: 703 - protein, 119 aa, synthetic construct
QVQLQQSGAEVKKPGSSVKVSCKASGGTFSSYAISWVRQAPGQGLEWMGGIIPILGIANYAQKFQGRVTITADESTSTAYMELSSLRSEDTAVYYCARGGSGSYHMDVWGKGTTVTVSS

SEQ ID NO: 704 - protein, 109 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPRITFGQGTRLEIK

SEQ ID NO: 705 - protein, 5 aa, synthetic construct
IYNVH

SEQ ID NO: 706 - protein, 17 aa, synthetic construct
TIFPGNGDTSYNQKFKD

SEQ ID NO: 707 - protein, 10 aa, synthetic construct
WDDGNVGFAH

SEQ ID NO: 708 - protein, 11 aa, synthetic construct
RASENINNYLT

SEQ ID NO: 709 - protein, 7 aa, synthetic construct
HAKTLAE

SEQ ID NO: 710 - protein, 9 aa, synthetic construct
QHHYGTPPT

SEQ ID NO: 711 - protein, 119 aa, synthetic construct
QVQLQQPGAELVKPGASVKMSCKASGYTFTIYNVHWIKQTPGQGLEWMGTIFPGNGDTSYNQKFKDKATLTTDKSSKTAYMQLNSLTSEDSAVYYCARWDDGNVGFAHWGQGTLVTVSA

SEQ ID NO: 712 - protein, 107 aa, synthetic construct
DIQMTQSPASLSASVGETVTITCRASENINNYLTWFQQKQGKSPQLLVYHAKTLAEGVPSRFSGSGSGTQFSLKINSLQPEDFGSYYCQHHYGTPPTFGGGTKLEIK

SEQ ID NO: 713 - protein, 119 aa, synthetic construct
EVQLVQSGAEVKKPGASVKVSCKASGYTFTIYNVHWVRQAPGQGLEWMGTIFPGNGDTSYNQKFKDKVTMTTDTSTSTAYMELSSLRSEDTAVYYCARWDDGNVGFAHWGQGTLVTVSS

SEQ ID NO: 714 - protein, 107 aa, synthetic construct
DIQMTQSPSSLSASVGDRVTITCRASENINNYLTWFQQKQGKSPQLLIYHAKTLAEGVPSRFSGSGSGTDFTLTISSLQPEDFATYYCQHHYGTPPTFGGGTKVEIK

SEQ ID NO: 715 - protein, 119 aa, synthetic construct
EVQLVQSGAEVKKPGASVKVSCKASGYTFTIYNVHWIRQAPGQGLEWMGTIFPGNGDTSYNQKFKDRATLTTDKSTKTAYMELRSLRSDDTAVYYCARWDDGNVGFAHWGQGTLVTVSS

SEQ ID NO: 716 - protein, 107 aa, synthetic construct
DIQMTQSPSSLSASVGDRVTITCRASENINNYLTWFQQKPGKAPKLLVYHAKTLAEGVPSRFSGSGSGTQFTLTISSLQPEDFATYYCQHHYGTPPTFGQGTKLEIK

SEQ ID NO: 717 - protein, 447 aa, synthetic construct
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 718 - protein, 220 aa, synthetic construct
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 719 - protein, 445 aa, synthetic construct
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 720 - protein, 217 aa, synthetic construct
QSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 721 - protein, 447 aa, synthetic construct
EVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 722 - protein, 214 aa, synthetic construct
DIQMTQSPSSLSASVGDRVTITCRASQGISNWLAWYQQKPGKAPKLLIYGASSLQSGVPSRFSGSGSGTDFTLTISSLQPEDFATYYCQQYSSFPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 723 - protein, 449 aa, synthetic construct
QVQLVQSGAEVKKPGASVKVSCKASGYTFRSSYISWVRQAPGQGLEWMGWIYAGTGSPSYNQKLQGRVTMTTDTSTSTAYMELRSLRSDDTAVYYCARHRDYYSNSLTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 724 - protein, 220 aa, synthetic construct
DIVMTQSPDSLAVSLGERATINCKSSQSVLNSGNQKNYLTWYQQKPGQPPKLLIYWASTRESGVPDRFSGSGSGTDFTLTISSLQAEDVAVYYCQSDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 725 - protein, 7 aa, synthetic construct
TSNMGVG

SEQ ID NO: 726 - protein, 16 aa, synthetic construct
HIWWDDDKYYSPSLKS

SEQ ID NO: 727 - protein, 10 aa, synthetic construct
SNYGYAWFAY

SEQ ID NO: 728 - protein, 11 aa, synthetic construct
KASQDIYPYLN

SEQ ID NO: 729 - protein, 7 aa, synthetic construct
RTNRLLD

SEQ ID NO: 730 - protein, 9 aa, synthetic construct
LQYDEFPLT

SEQ ID NO: 731 - protein, 120 aa, synthetic construct
QITLKESGPTLVKPTQTLTLTCTFSGFSLSTSNMGVGWIRQPPGKALEWLAHIWWDDDKYYSPSLKSRLTITKDTSKNQVVLTMTNMDPVDTATYYCVRSNYGYAWFAYWGQGTLVTVSS

SEQ ID NO: 732 - protein, 107 aa, synthetic construct
DIQMTQSPSSLSASVGDRVTITCKASQDIYPYLNWFQQKPGKAPKTLIYRTNRLLDGVPSRFSGSGSGTDFTFTISSLQPEDIATYYCLQYDEFPLTFGAGTKLEIK

SEQ ID NO: 733 - protein, 5 aa, synthetic construct
DYAVH

SEQ ID NO: 734 - protein, 17 aa, synthetic construct
VISTYNDYTYNNQDFKG

SEQ ID NO: 735 - protein, 10 aa, synthetic construct
GNSYFYALDY

SEQ ID NO: 736 - protein, 15 aa, synthetic construct
RASESVDSYGKSFMH

SEQ ID NO: 737 - protein, 7 aa, synthetic construct
RASNLES

SEQ ID NO: 738 - protein, 9 aa, synthetic construct
QQSNEDPWT

SEQ ID NO: 739 - protein, 119 aa, synthetic construct
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 740 - protein, 111 aa, synthetic construct
EIVLTQSPATLSLSPGERATLSCRASESVDSYGKSFMHWYQQKPGQAPRLLIYRASNLESGIPARFSGSGSGTDFTLTISSLEPEDFAVYYCQQSNEDPWTFGGGTKLEIK

SEQ ID NO: 741 - protein, 5 aa, synthetic construct
RYWMS

SEQ ID NO: 742 - protein, 17 aa, synthetic construct
DLNPDSSAINYVDSVKG

SEQ ID NO: 743 - protein, 11 aa, synthetic construct
ITTLVPYTMDF

SEQ ID NO: 744 - protein, 11 aa, synthetic construct
ITNTDIDDDMN

SEQ ID NO: 745 - protein, 7 aa, synthetic construct
EGNGLRP

SEQ ID NO: 746 - protein, 9 aa, synthetic construct
LQSDNLPLT

SEQ ID NO: 747 - protein, 120 aa, synthetic construct
EVQLVESGGGLVQPGGSLRLSCAASGFDFSRYWMSWVRQAPGKGLEWIGDLNPDSSAINYVDSVKGRFTISRDNAKNSLYLQMNSLRAEDTAVYYCTLITTLVPYTMDFWGQGTSVTVSS

SEQ ID NO: 748 - protein, 107 aa, synthetic construct
ETTLTQSPAFMSATPGDKVNISCITNTDIDDDMNWYQQKPGEAAILLISEGNGLRPGIPPRFSGSGYGTDFTLTINNIESEDAAYYFCLQSDNLPLTFGSGTKLEIK

SEQ ID NO: 749 - protein, 5 aa, synthetic construct
DYYMH

SEQ ID NO: 750 - protein, 17 aa, synthetic construct
WIDPENGDTEYGPKFQG

SEQ ID NO: 751 - protein, 11 aa, synthetic construct
HNAHYGTWFAY

SEQ ID NO: 752 - protein, 16 aa, synthetic construct
RSSQSLLHSSGNTYLE

SEQ ID NO: 753 - protein, 7 aa, synthetic construct
KISTRFS

SEQ ID NO: 754 - protein, 9 aa, synthetic construct
FQGSHVPYT

SEQ ID NO: 755 - protein, 120 aa, synthetic construct
QVQLVQSGAEVKKPGASVKVSCKASGLTIEDYYMHWVRQAPGQGLEWMGWIDPENGDTEYGPKFQGRVTMTRDTSINTAYMELSRLRSDDTAVYYCAVHNAHYGTWFAYWGQGTLVTVSS

SEQ ID NO: 756 - protein, 112 aa, synthetic construct
DVVMTQSPLSLPVTLGQPASISCRSSQSLLHSSGNTYLEWYQQRPGQSPRPLIYKISTRFSGVPDRFSGSGSGTDFTLKISRVEAEDVGVYYCFQGSHVPYTFGGGTKVEIK

SEQ ID NO: 757 - protein, 449 aa, synthetic construct
QVQLVQSGAEVKKPGASVKVSCKASGLTIEDYYMHWVRQAPGQGLEWMGWIDPENGDTEYGPKFQGRVTMTRDTSINTAYMELSRLRSDDTAVYYCAVHNAHYGTWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 758 - protein, 219 aa, synthetic construct
DVVMTQSPLSLPVTLGQPASISCRSSQSLLHSSGNTYLEWYQQRPGQSPRPLIYKISTRFSGVPDRFSGSGSGTDFTLKISRVEAEDVGVYYCFQGSHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 759 - protein, 5 aa, synthetic construct
GYFMN

SEQ ID NO: 760 - protein, 17 aa, synthetic construct
LINPYNGDSFYNQKFKG

SEQ ID NO: 761 - protein, 8 aa, synthetic construct
GLRRDFDY

SEQ ID NO: 762 - protein, 16 aa, synthetic construct
KSSQSLLDSDGKTYLN

SEQ ID NO: 763 - protein, 7 aa, synthetic construct
LVSELDS

SEQ ID NO: 764 - protein, 9 aa, synthetic construct
WQGTHFPRT

SEQ ID NO: 765 - protein, 117 aa, synthetic construct
QVQLVQSGAEVKKPGASVKVSCKASGYSFSGYFMNWVRQAPGQGLEWMGLINPYNGDSFYNQKFKGRVTMTRQTSTSTVYMELSSLRSEDTAVYYCVRGLRRDFDYWGQGTLVTVSS

SEQ ID NO: 766 - protein, 112 aa, synthetic construct
DVVMTQSPLSLPVTLGQPASISCKSSQSLLDSDGKTYLNWLFQRPGQSPRRLIYLVSELDSGVPDRFSGSGSGTDFTLKISRVEAEDVGVYYCWQGTHFPRTFGGGTKLEIK

SEQ ID NO: 767 - protein, 5 aa, synthetic construct
DFGMN

SEQ ID NO: 768 - protein, 17 aa, synthetic construct
WINTFTGEPSYGNVFKG

SEQ ID NO: 769 - protein, 10 aa, synthetic construct
RHGNGNVFDS

SEQ ID NO: 770 - protein, 11 aa, synthetic construct
RASQSIGSNIH

SEQ ID NO: 771 - protein, 7 aa, synthetic construct
YTSESIS

SEQ ID NO: 772 - protein, 9 aa, synthetic construct
QQSNSWPLT

SEQ ID NO: 773 - protein, 119 aa, synthetic construct
QVQLVQSGSELKKPGASVKVSCKASGYTFTDFGMNWVRQAPGQGLEWMGWINTFTGEPSYGNVFKGRFVFSLDTSVSTAYLQISSLKAEDTAVYYCARRHGNGNVFDSWGQGTLVTVSS

SEQ ID NO: 774 - protein, 108 aa, synthetic construct
EIVLTQSPDFQSVTPKEKVTITCRASQSIGSNIHWYQQKPDQSPKLLIKYTSESISGVPSRFSGSGSGTDFTLTINSLEAEDAATYYCQQSNSWPLTFGGGTKVEIKR

SEQ ID NO: 775 - protein, 5 aa, synthetic construct
SYAIS

SEQ ID NO: 776 - protein, 17 aa, synthetic construct
GIIPIFGTANYAQKFQG

SEQ ID NO: 777 - protein, 21 aa, synthetic construct
APLRFLEWSTQDHYYYYYMDV

SEQ ID NO: 778 - protein, 11 aa, synthetic construct
QGDSLRSYYAT

SEQ ID NO: 779 - protein, 7 aa, synthetic construct
GENKRPS

SEQ ID NO: 780 - protein, 11 aa, synthetic construct
KSRDGSGQHLV

SEQ ID NO: 781 - protein, 130 aa, synthetic construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSS

SEQ ID NO: 782 - protein, 108 aa, synthetic construct
SSELTQDPAVSVALGQTVRITCQGDSLRSYYATWYQQKPGQAPILVIYGENKRPSGIPDRFSGSSSGNTASLTITGAQAEDEADYYCKSRDGSGQHLVFGGGTKLTVL

SEQ ID NO: 783 - protein, 5 aa, synthetic construct
SYWIN

SEQ ID NO: 784 - protein, 17 aa, synthetic construct
NIYPSDSYTNYNQKFKD

SEQ ID NO: 785 - protein, 9 aa, synthetic construct
SWRGNSFDY

SEQ ID NO: 786 - protein, 17 aa, synthetic construct
KSSQSLLNSGNQKNYLT

SEQ ID NO: 787 - protein, 7 aa, synthetic construct
WASTRES

SEQ ID NO: 788 - protein, 9 aa, synthetic construct
QNDYSYPFT

SEQ ID NO: 789 - protein, 118 aa, synthetic construct
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 790 - protein, 113 aa, synthetic construct
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 791 - protein, 5 aa, synthetic construct
NYGMN

SEQ ID NO: 792 - protein, 17 aa, synthetic construct
WINTNTGEPTYAEEFKG

SEQ ID NO: 793 - protein, 9 aa, synthetic construct
LGFGNAMDY

SEQ ID NO: 794 - protein, 17 aa, synthetic construct
KSSQSLLNSGNQKNYLT

SEQ ID NO: 795 - protein, 7 aa, synthetic construct
WASTRES

SEQ ID NO: 796 - protein, 9 aa, synthetic construct
QNDYSYPLT

SEQ ID NO: 797 - protein, 118 aa, synthetic construct
QIQLVQSGPELKKPGETVKISCKASGYTFTNYGMNWVKQAPGKGLKWMGWINTNTGEPTYAEEFKGRFAFSLETSASTAYLQINNLKNEDTATYFCARLGFGNAMDYWGQGTSVTVSS

SEQ ID NO: 798 - protein, 113 aa, synthetic construct
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELK

SEQ ID NO: 799 - protein, 5 aa, synthetic construct
SYNMN

SEQ ID NO: 800 - protein, 17 aa, synthetic construct
YISSSSSTIYYADSVKG

SEQ ID NO: 801 - protein, 8 aa, synthetic construct
AYYYGMDV

SEQ ID NO: 802 - protein, 11 aa, synthetic construct
RASQGISGWLA

SEQ ID NO: 803 - protein, 7 aa, synthetic construct
AASTLQS

SEQ ID NO: 804 - protein, 9 aa, synthetic construct
QQANSFPPT

SEQ ID NO: 805 - protein, 117 aa, synthetic construct
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 806 - protein, 107 aa, synthetic construct
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIK

SEQ ID NO: 807 - protein, 5 aa, synthetic construct
SYGMH

SEQ ID NO: 808 - protein, 17 aa, synthetic construct
VIWYDGSNQYYADSVKG

SEQ ID NO: 809 - protein, 11 aa, synthetic construct
GLTSGRYGMDV

SEQ ID NO: 810 - protein, 16 aa, synthetic construct
RSSQSLLLSHGFNYLD

SEQ ID NO: 811 - protein, 7 aa, synthetic construct
LGSSRAS

SEQ ID NO: 812 - protein, 9 aa, synthetic construct
MQPLQIPWT

SEQ ID NO: 813 - protein, 120 aa, synthetic construct
QVQLVESGGGVVQPGRSLRLSCAASGFTFSSYGMHWVRQAPGKGLEWVAVIWYDGSNQYYADSVKGRFTISRDNSKNTLFLQMHSLRAEDTAVYYCARGLTSGRYGMDVWGQGTTVTVSS

SEQ ID NO: 814 - protein, 112 aa, synthetic construct
DIVMTQSPLSLPVTPGEPASISCRSSQSLLLSHGFNYLDWYLQKPGQSPQLLIYLGSSRASGVPDRFSGSGSGTDFTLKISRVEAEDVGLYYCMQPLQIPWTFGQGTKVEIK

SEQ ID NO: 815 - protein, 5 aa, synthetic construct
NYAMS

SEQ ID NO: 816 - protein, 17 aa, synthetic construct
SISGSGDYTYYTDSVKG

SEQ ID NO: 817 - protein, 9 aa, synthetic construct
SPWGYYLDS

SEQ ID NO: 818 - protein, 11 aa, synthetic construct
RASQGISSRLA

SEQ ID NO: 819 - protein, 7 aa, synthetic construct
AASSLQS

SEQ ID NO: 820 - protein, 9 aa, synthetic construct
QQYNSYPYT

SEQ ID NO: 821 - protein, 118 aa, synthetic construct
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSS

SEQ ID NO: 822 - protein, 107 aa, synthetic construct
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIK

SEQ ID NO: 823 - protein, 5 aa, synthetic construct
DHAIH

SEQ ID NO: 824 - protein, 17 aa, synthetic construct
YFSPGNDDIKYNEKFRG

SEQ ID NO: 825 - protein, 6 aa, synthetic construct
SLSTPY

SEQ ID NO: 826 - protein, 17 aa, synthetic construct
KSSQSLLNRGNHKNYLT

SEQ ID NO: 827 - protein, 7 aa, synthetic construct
WASTRES

SEQ ID NO: 828 - protein, 9 aa, synthetic construct
QNDYTYPYT

SEQ ID NO: 829 - protein, 115 aa, synthetic construct
EVQLVQSGAEVKKPGASVKVSCKASGYTFTDHAIHWVRQAPGQGLEWMGYFSPGNDDIKYNEKFRGRVTMTADKSSSTAYMELRSLRSDDTAVYFCKRSLSTPYWGQGTLVTVSS

SEQ ID NO: 830 - protein, 113 aa, synthetic construct
DIVMTQSPDSLAVSLGERATINCKSSQSLLNRGNHKNYLTWYQQKPGQPPKLLIYWASTRESGVPDRFSGSGSGTDFTLTISSLQAEDVAVYYCQNDYTYPYTFGQGTKVEIK

SEQ ID NO: 831 - protein, 5 aa, synthetic construct
SYNMH

SEQ ID NO: 832 - protein, 17 aa, synthetic construct
AIYPGNGDTSYNQKFKG

SEQ ID NO: 833 - protein, 12 aa, synthetic construct
STYYGGDWYFNV

SEQ ID NO: 834 - protein, 10 aa, synthetic construct
RASSSVSYIH

SEQ ID NO: 835 - protein, 7 aa, synthetic construct
ATSNLAS

SEQ ID NO: 836 - protein, 9 aa, synthetic construct
QQWTSNPPT

SEQ ID NO: 837 - protein, 121 aa, synthetic construct [sequence omitted]

SEQ ID NO: 838 - protein, 106 aa, synthetic construct [sequence omitted]

SEQ ID NO: 839 - protein, 5 aa, synthetic construct
DTYIH

SEQ ID NO: 840 - protein, 17 aa, synthetic construct
RIYPTNGYTRYADSVKG

SEQ ID NO: 841 - protein, 11 aa, synthetic construct
WGGDGFYAMDY

SEQ ID NO: 842 - protein, 11 aa, synthetic construct
RASQDVNTAVA

SEQ ID NO: 843 - protein, 7 aa, synthetic construct
SASFLYS

SEQ ID NO: 844 - protein, 9 aa, synthetic construct
QQHYTTPPT

SEQ ID NO: 845 - protein, 120 aa, synthetic construct [sequence omitted]

SEQ ID NO: 846 - protein, 107 aa, synthetic construct [sequence omitted]

SEQ ID NO: 847 - protein, 5 aa, synthetic construct
SYWIE

SEQ ID NO: 848 - protein, 17 aa, synthetic construct
EILPGGGDTNYNEIFKG

SEQ ID NO: 849 - protein, 8 aa, synthetic construct
RVPIRLDY

SEQ ID NO: 850 - protein, 15 aa, synthetic construct
KASQSVDYEGDSFLN

SEQ ID NO: 851 - protein, 7 aa, synthetic construct
AASNLES

SEQ ID NO: 852 - protein, 9 aa, synthetic construct
QQSNEDPLT

SEQ ID NO: 853 - protein, 117 aa, synthetic construct [sequence omitted]

SEQ ID NO: 854 - protein, 111 aa, synthetic construct [sequence omitted]

SEQ ID NO: 855 - protein, 5 aa, synthetic construct
DFAMS

SEQ ID NO: 856 - protein, 17 aa, synthetic construct
TIGRVAFHTYYPDSMKG

SEQ ID NO: 857 - protein, 11 aa, synthetic construct
HRGFDVGHFDF

SEQ ID NO: 858 - protein, 16 aa, synthetic construct
RSSETLVHSSGNTYLE

SEQ ID NO: 859 - protein, 7 aa, synthetic construct
RVSNRFS

SEQ ID NO: 860 - protein, 9 aa, synthetic construct
FQGSFNPLT

SEQ ID NO: 861 - protein, 120 aa, synthetic construct
EVQLVESGGGLVQPGGSLRLSCAASGFSFSDFAMSWVRQAPGKGLEWVATIGRVAFHTYYPDSMKGRFTISRDNSKNTLYLQMNSLRAEDTAVYYCARHRGFDVGHFDFWGQGTLVTVSS

SEQ ID NO: 862 - protein, 112 aa, synthetic construct
DIQMTQSPSSLSASVGDRVTITCRSSETLVHSSGNTYLEWYQQKPGKAPKLLIYRVSNRFSGVPSRFSGSGSGTDFTLTISSLQPEDFATYYCFQGSFNPLTFGQGTKVEIK

SEQ ID NO: 863 - protein, 6 aa, synthetic construct
NDYAWN

SEQ ID NO: 864 - protein, 16 aa, synthetic construct
YISYSGYTTYNPSLKS

SEQ ID NO: 865 - protein, 7 aa, synthetic construct
WTSGLDY

SEQ ID NO: 866 - protein, 11 aa, synthetic construct
KASDLIHNWLA

SEQ ID NO: 867 - protein, 7 aa, synthetic construct
GATSLET

SEQ ID NO: 868 - protein, 9 aa, synthetic construct
QQYWTTPFT

SEQ ID NO: 869 - protein, 116 aa, synthetic construct
EVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWTSGLDYWGQGTLVTVSS

SEQ ID NO: 870 - protein, 107 aa, synthetic construct
DIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIK

SEQ ID NO: 871 - protein, 6 aa, synthetic construct
SDYAWN

SEQ ID NO: 872 - protein, 16 aa, synthetic construct
YISNSGSTSYNPSLKS

SEQ ID NO: 873 - protein, 15 aa, synthetic construct
ERNYDYDDYYYAMDY

SEQ ID NO: 874 - protein, 17 aa, synthetic construct
KSSQSLLYRSNQKNYLA

SEQ ID NO: 875 - protein, 7 aa, synthetic construct
WASTRES

SEQ ID NO: 876 - protein, 9 aa, synthetic construct
QQYYNYPRT

SEQ ID NO: 877 - protein, 124 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSS

SEQ ID NO: 878 - protein, 113 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIK

SEQ ID NO: 879 - protein, 5 aa, synthetic construct
NYWMH

SEQ ID NO: 880 - protein, 17 aa, synthetic construct
ATYRGHSDTYYNQKFKG

SEQ ID NO: 881 - protein, 12 aa, synthetic construct
GAIYDGYDVLDN

SEQ ID NO: 882 - protein, 11 aa, synthetic construct
SASQDISNYLN

SEQ ID NO: 883 - protein, 7 aa, synthetic construct
YTSNLHS

SEQ ID NO: 884 - protein, 9 aa, synthetic construct
QQYRKLPWT

SEQ ID NO: 885 - protein, 121 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 886 - protein, 107 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIK

SEQ ID NO: 887 - protein, 5 aa, synthetic construct
AYTMH

SEQ ID NO: 888 - protein, 17 aa, synthetic construct
WIKPNNGLANYAQKFQG

SEQ ID NO: 889 - protein, 9 aa, synthetic construct
SEITTEFDY

SEQ ID NO: 890 - protein, 15 aa, synthetic construct
KSSESVDSYANSFLH

SEQ ID NO: 891 - protein, 7 aa, synthetic construct
RASTRES

SEQ ID NO: 892 - protein, 9 aa, synthetic construct
QQSKEDPLT

SEQ ID NO: 893 - protein, 118 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSS

SEQ ID NO: 894 - protein, 111 aa, synthetic construct
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIK

SEQ ID NO: 895 - protein, 6 aa, synthetic construct
SDFAWN

SEQ ID NO: 896 - protein, 16 aa, synthetic construct
YISYSGNTRYQPSLKS

SEQ ID NO: 897 - protein, 7 aa, synthetic construct
AGRGFPY

SEQ ID NO: 898 - protein, 11 aa, synthetic construct
HSSQDINSNIG

SEQ ID NO: 899 - protein, 7 aa, synthetic construct
HGTNLDD

SEQ ID NO: 900 - protein, 9 aa, synthetic construct
VQYAQFPWT

SEQ ID NO: 901 - protein, 116 aa, synthetic construct
QVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSS

SEQ ID NO: 902 - protein, 107 aa, synthetic construct; identical to SEQ ID NO: 574

SEQ ID NO: 903 - protein, 5 aa, synthetic construct
DYYMA

SEQ ID NO: 904 - protein, 17 aa, synthetic construct
SINYDGSSTYYVDSVKG

SEQ ID NO: 905 - protein, 8 aa, synthetic construct
DRGYYFDY

SEQ ID NO: 906 - protein, 16 aa, synthetic construct
RSSQSLVHSNGNTYLH

SEQ ID NO: 907 - protein, 7 aa, synthetic construct
KVSNRFS

SEQ ID NO: 908 - protein, 10 aa, synthetic construct
SQSTHVPPFT

SEQ ID NO: 909 - protein, 117 aa, synthetic construct
EVQLVESGGGLVQPGGSLRLSCAASGFTFSDYYMAWVRQAPGKGLEWVASINYDGSSTYYVDSVKGRFTISRDNAKNSLYLQMNSLRAEDTAVYYCARDRGYYFDYWGQGTTVTVSS

SEQ ID NO: 910 - protein, 113 aa, synthetic construct
DVVMTQTPLSLSVTPGQPASISCRSSQSLVHSNGNTYLHWYLQKPGQSPQLLIYKVSNRFSGVPDRFSGSGSGTDFTLKISRVEAEDVGVYFCSQSTHVPPFTFGGGTKVEIK

SEQ ID NO: 911 - protein, 5 aa, synthetic construct
NAWMS

SEQ ID NO: 912 - protein, 17 aa, synthetic construct
YISSSGSTIYYADSVKG

SEQ ID NO: 913 - protein, 8 aa, synthetic construct
EGLWAFDY

SEQ ID NO: 914 - protein, 14 aa, synthetic construct
TGSSSNIGAGYVVH

SEQ ID NO: 915 - protein, 7 aa, synthetic construct
DNNKRPS

SEQ ID NO: 916 - protein, 11 aa, synthetic construct
AAWDDRLNGPV

SEQ ID NO: 917 - protein, 117 aa, synthetic construct
EVQLLESGGGLVQPGGSLRLSCAASGFTFSNAWMSWVRQAPGKGLEWVSYISSSGSTIYYADSVKGRFTISRDNSKNTLYLQMNSLRAEDTAVYYCAREGLWAFDYWGQGTLVTVSS

SEQ ID NO: 918 - protein, 111 aa, synthetic construct
ESVLTQPPSVSGAPGQRVTISCTGSSSNIGAGYVVHWYQQLPGTAPKLLIYDNNKRPSGVPDRFSGSKSGTSASLAISGLRSEDEADYYCAAWDDRLNGPVFGGGTKLTVL

SEQ ID NO: 919 - protein, 5 aa, synthetic construct
GYYWS

SEQ ID NO: 920 - protein, 16 aa, synthetic construct
EINHRGNTNDNPSLKS

SEQ ID NO: 921 - protein, 11 aa, synthetic construct
ERGYTYGNFDH

SEQ ID NO: 922 - protein, 11 aa, synthetic construct
RASQSVSRNLA

SEQ ID NO: 923 - protein, 7 aa, synthetic construct
GASTRAT

SEQ ID NO: 924 - protein, 9 aa, synthetic construct
QQYKTWPRT

SEQ ID NO: 925 - protein, 119 aa, synthetic construct
QVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSS

SEQ ID NO: 926 - protein, 107 aa, synthetic construct
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIK

SEQ ID NO: 927 - protein, 5 aa, synthetic construct
SYAMN

SEQ ID NO: 928 - protein, 17 aa, synthetic construct
TTSGSGASTYYADSVKG

SEQ ID NO: 929 - protein, 7 aa, synthetic construct
IWIAFDI

SEQ ID NO: 930 - protein, 12 aa, synthetic construct
RASQSVSSSYLA

SEQ ID NO: 931 - protein, 7 aa, synthetic construct
GASSRAT

SEQ ID NO: 932 - protein, 9 aa, synthetic construct
QQYGSSPYT

SEQ ID NO: 933 - protein, 116 aa, synthetic construct
EVQLLESGGGLVQPGGSLRLSCAASGFTFSSYAMNWVRQAPGKGLEWVSTTSGSGASTYYADSVKGRFTISRDNSKNTLYLQMNSLRAEDTAVYYCAKIWIAFDIWGQGTMVTVSS

SEQ ID NO: 934 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKLEIK

SEQ ID NO: 935 - protein, 7 aa, synthetic construct
SFNYYWS

SEQ ID NO: 936 - protein, 16 aa, synthetic construct
YIYYSGSTYSNPSLKS

SEQ ID NO: 937 - protein, 9 aa, synthetic construct
GYNWNYFDY

SEQ ID NO: 938 - protein, 11 aa, synthetic construct
RASQSVDNNLV

SEQ ID NO: 939 - protein, 7 aa, synthetic construct
GASTRAT

SEQ ID NO: 940 - protein, 10 aa, synthetic construct [sequence omitted]

SEQ ID NO: 941 - protein, 119 aa, synthetic construct [sequence omitted]

SEQ ID NO: 942 - protein, 108 aa, synthetic construct [sequence omitted]

SEQ ID NO: 943 - protein, 449 aa, synthetic construct
QVQLQESGPGLVKPSQTLSLTCTVSGGSISSFNYYWSWIRHHPGKGLEWIGYIYYSGSTYSNPSLKSRVTISVDTSKNQFSLTLSSVTAADTAVYYCARGYNWNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 944 - protein, 215 aa, synthetic construct
EIVMTQSPATLSVSPGERATLSCRASQSVDNNLVWYQQKPGQAPRLLIYGASTRATGIPARFSGSGSGTEFTLTISSLQSEDFAVYYCQQYNNWPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 945 - protein, 5 aa, synthetic construct
TYWMH

SEQ ID NO: 946 - protein, 17 aa, synthetic construct
EIDPSDSYSNYNQKFKD

SEQ ID NO: 947 - protein, 11 aa, synthetic construct
NGGLGPAWFSY

SEQ ID NO: 948 - protein, 11 aa, synthetic construct
KASQYVGTAVA

SEQ ID NO: 949 - protein, 7 aa, synthetic construct
SASNRYT

SEQ ID NO: 950 - protein, 9 aa, synthetic construct
QQYSSYPWT

SEQ ID NO: 951 - protein, 120 aa, synthetic construct
EVQLVQSGAEVKKPGSSVKVSCKASGYTFTTYWMHWVRQAPGQGLEWIGEIDPSDSYSNYNQKFKDRATLTVDKSTSTAYMELSSLRSEDTAVYYCARNGGLGPAWFSYWGQGTLVTVSS

SEQ ID NO: 952 - protein, 107 aa, synthetic construct
DIQMTQSPSSVSASVGDRVTITCKASQYVGTAVAWYQQKPGKSPKLLIYSASNRYTGVPSRFSDSGSGTDFTLTISSLQPEDFATYFCQQYSSYPWTFGGGTKVEIK

SEQ ID NO: 953 - protein, 5 aa, synthetic construct
SYAMS

SEQ ID NO: 954 - protein, 17 aa, synthetic construct
AISGSGTSTYYADSVKG

SEQ ID NO: 955 - protein, 13 aa, synthetic construct
VRYNWNHGDWFDP

SEQ ID NO: 956 - protein, 13 aa, synthetic construct
SGSSSNIGNNYVS

SEQ ID NO: 957 - protein, 7 aa, synthetic construct
ENYNRPA

SEQ ID NO: 958 - protein, 11 aa, synthetic construct
SSWDDSLNYWV

SEQ ID NO: 959 - protein, 122 aa, synthetic construct
EVQLLESGGGLVQPGGSLRLSCAASGFTFSSYAMSWVRQAPGKGLEWVSAISGSGTSTYYADSVKGRFTISRDNSKNTLYLQMNSLRAEDTAVYYCARVRYNWNHGDWFDPWGQGTLVTVSS

SEQ ID NO: 960 - protein, 110 aa, synthetic construct
QSVLTQPPSASGTPGQRVTISCSGSSSNIGNNYVSWYQQLPGTAPKLLIYENYNRPAGVPDRFSGSKSGTSASLAISGLRSEDEADYYCSSWDDSLNYWVFGGGTKLTVL

SEQ ID NO: 961 - protein, 5 aa, synthetic construct
SYGMS

SEQ ID NO: 962 - protein, 17 aa, synthetic construct
TISSGGSYKYYVDSVKG

SEQ ID NO: 963 - protein, 11 aa, synthetic construct
HPDYDGVWFAY

SEQ ID NO: 964 - protein, 10 aa, synthetic construct
SVSSSVFYVH

SEQ ID NO: 965 - protein, 7 aa, synthetic construct
DTSKLAS

SEQ ID NO: 966 - protein, 9 aa, synthetic construct
QQWNSNPPT

SEQ ID NO: 967 - protein, 120 aa, synthetic construct
EVQLVESGGGLVQPGGSLRLSCAASGFTFNSYGMSWVRQAPGKGLEWVATISSGGSYKYYVDSVKGRFTISRDNAKNSLYLQMNSLRAEDTAVYYCARHPDYDGVWFAYWGQGTLVTVSS

SEQ ID NO: 968 - protein, 106 aa, synthetic construct
DIQMTQSPSSLSASVGDRVTITCSVSSSVFYVHWYQQKPGKAPKLLIYDTSKLASGVPSRFSGSGSGTDFTFTISSLQPEDIATYYCQQWNSNPPTFGGGTKVEIK

SEQ ID NO: 969 - protein, 5 aa, synthetic construct
SYGMS

SEQ ID NO: 970 - protein, 17 aa, synthetic construct
TISSGGSYTYYPDSVKG

SEQ ID NO: 971 - protein, 11 aa, synthetic construct
HPDYDGVWFAY

SEQ ID NO: 972 - protein, 10 aa, synthetic construct
SVSSSVFYVH

SEQ ID NO: 973 - protein, 7 aa, synthetic construct
DTSKLAS

SEQ ID NO: 974 - protein, 9 aa, synthetic construct
QQWNSNPPT

SEQ ID NO: 975 - protein, 120 aa, synthetic construct
EVQLVESGGDLVKPGGSLKLSCAASGFTFNSYGMSWVRQTPDKRLEWVATISSGGSYTYYPDSVKGRFTISRDNAKNTLYLQMSSLKSEDTAMYYCARHPDYDGVWFAYWGQGTLVTVSA

SEQ ID NO: 976 - protein, 106 aa, synthetic construct
QIVLTQSPAIMSASPGEKVTMTCSVSSSVFYVHWYQQKSGTSPKRWIYDTSKLASGVPARFSGSGSGTSYSLTISSMEAEDAATYYCQQWNSNPPTFGGGTKLEIK

SEQ ID NO: 977 - protein, 5 aa, synthetic construct
SYYMH

SEQ ID NO: 978 - protein, 17 aa, synthetic construct
IINPSGGSTSYAQKFQG

SEQ ID NO: 979 - protein, 19 aa, synthetic construct
DGVLRYFDWLLDYYYYMDV

SEQ ID NO: 980 - protein, 11 aa, synthetic construct
RASQSVGSYLA

SEQ ID NO: 981 - protein, 7 aa, synthetic construct
DASNRAT

SEQ ID NO: 982 - protein, 8 aa, synthetic construct
QQRANVFT

SEQ ID NO: 983 - protein, 128 aa, synthetic construct
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDGVLRYFDWLLDYYYYMDVWGKGTTVTVSS

SEQ ID NO: 984 - protein, 106 aa, synthetic construct
EIVLTQSPATLSLSPGERATLSCRASQSVGSYLAWYQQRPGQAPRLLIYDASNRATGIPARFSGSGSGTDFTLTISSLEPEDFAVYYCQQRANVFTFGQGTKVEIK

SEQ ID NO: 985 - protein, 5 aa, synthetic construct
SYYMH

SEQ ID NO: 986 - protein, 17 aa, synthetic construct
IINPSGGSTSYAQKFQG

SEQ ID NO: 987 - protein, 19 aa, synthetic construct
DAELRHFDHLLDYHYYMDV

SEQ ID NO: 988 - protein, 11 aa, synthetic construct
RASQSVGSYLA

SEQ ID NO: 989 - protein, 7 aa, synthetic construct
DASNRAT

SEQ ID NO: 990 - protein, 8 aa, synthetic construct
QQRAQEFT

SEQ ID NO: 991 - protein, 128 aa, synthetic construct
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDAELRHFDHLLDYHYYMDVWGQGTTVTVSS

SEQ ID NO: 992 - protein, 106 aa, synthetic construct
EIVMTQSPATLSLSPGERATLSCRASQSVGSYLAWYQQKPGQAPRLLIYDASNRATGIPARFSGSGSGTDFTLTISSLQPEDFAVYYCQQRAQEFTFGQGTKVEIK

SEQ ID NO: 993 - protein, 120 aa, synthetic construct
QVQLVQSGSELKKPGAPVKVSCKASGYTFSTFGMSWVRQAPGQGLKWMGWIHTYAGVPIYGDDFKGRFVFSLDTSVSTAYLQISSLKAEDTAVYFCARRSDNYRYFFDYWGQGTTVTVSS

SEQ ID NO: 994 - protein, 107 aa, synthetic construct
DIQMTQSPSSLSASLGDRVTITCRASQDIRNYLNWYQQKPGKAPKLLIYYTSRLHSGVPSRFSGSGSGTDYTLTISSLQPEDFATYFCQQGHTLPPTFGQGTKLEIK

SEQ ID NO: 995 - protein, 120 aa, synthetic construct
QIQLVQSGPELKKPGAPVKISCKASGYTFTTFGMSWVKQAPGQGLKWMGWIHTYAGVPIYGDDFKGRFVFSLDTSVSTAYLQISSVKAEDTATYFCARRSDNYRYFFDYWGQGTTLTVSS

SEQ ID NO: 996 - protein, 107 aa, synthetic construct; identical to SEQ ID NO: 994

SEQ ID NO: 997 - protein, 5 aa, synthetic construct
NYYMA

SEQ ID NO: 998 - protein, 17 aa, synthetic construct
SITKGGGNTYYRDSVKG

SEQ ID NO: 999 - protein, 14 aa, synthetic construct
QVTIAAVSTSYFDS

SEQ ID NO: 1000 - protein, 15 aa, synthetic construct
KTNQKVDYYGNSYVY

SEQ ID NO: 1001 - protein, 7 aa, synthetic construct
LASNLAS

SEQ ID NO: 1002 - protein, 9 aa, synthetic construct
QQSRNLPYT

SEQ ID NO: 1003 - protein, 123 aa, synthetic construct
EVQLVESGGGLVQSGRSIRLSCAASGFSFSNYYMAWVRQAPSKGLEWVASITKGGGNTYYRDSVKGRFTFSRDNAKSTLYLQMDSLRSEDTATYYCARQVTIAAVSTSYFDSWGQGVMVTVSS

SEQ ID NO: 1004 - protein, 110 aa, synthetic construct
DIVLTQSPALAVSLGQRATISCKTNQKVDYYGNSYVYWYQQKPGQQPKLLIYLASNLASGIPARFSGRGSGTDFTLTIDPVEADDTATYYCQQSRNLPYTFGAGTKLELK

SEQ ID NO: 1005 - protein, 5 aa, synthetic construct
DYYMK

SEQ ID NO: 1006 - protein, 17 aa, synthetic construct
DIIPSNGATFYNQKFKG

SEQ ID NO: 1007 - protein, 11 aa, synthetic construct
SHLLRASWFAY

SEQ ID NO: 1008 - protein, 17 aa, synthetic construct
KSSQSLLNSGNQKNYLT

SEQ ID NO: 1009 - protein, 7 aa, synthetic construct
WASTRES

SEQ ID NO: 1010 - protein, 9 aa, synthetic construct
QNDYSYPYT

SEQ ID NO: 1011 - protein, 120 aa, synthetic construct
QVQLVQSGAEVKKPGASVKMSCKASGYTFTDYYMKWVKQAPGQGLEWIGDIIPSNGATFYNQKFKGKATLTVDRSISTAYMHLNRLRSDDTAVYYCTRSHLLRASWFAYWGQGTLVTVSS

SEQ ID NO: 1012 - protein, 113 aa, synthetic construct
DFVMTQSPDSLAVSLGERATINCKSSQSLLNSGNQKNYLTWYLQKPGQPPKLLIYWASTRESGVPDRFSGSGSGTDFTLTISSLQAEDVAVYYCQNDYSYPYTFGQGTKLEIK

SEQ ID NO: 1013 - protein, 5 aa, synthetic construct
DYEMH

SEQ ID NO: 1014 - protein, 17 aa, synthetic construct
GIDPETGGTAYNQKFKG

SEQ ID NO: 1015 - protein, 6 aa, synthetic construct
YYSFAY

SEQ ID NO: 1016 - protein, 16 aa, synthetic construct
RSSQSIVHSNANTYLQ

SEQ ID NO: 1017 - protein, 7 aa, synthetic construct
KVSNRFS

SEQ ID NO: 1018 - protein, 9 aa, synthetic construct
FQVSHVPYT

SEQ ID NO: 1019 - protein, 115 aa, synthetic construct
EVQLVQSGAEVKKPGATVKISCKVSGYTFTDYEMHWVQQAPGKGLEWMGGIDPETGGTAYNQKFKGRVTLTADKSTDTAYMELSSLRSEDTAVYYCGRYYSFAYWGQGTLVTVSS

SEQ ID NO: 1020 - protein, 112 aa, synthetic construct
DVVMTQSPLSLPVTLGQPASISCRSSQSIVHSNANTYLQWFQQRPGQSPRLLIYKVSNRFSGVPDRFSGSGSGTDFTLKISRVEAEDVGVYYCFQVSHVPYTFGQGTKLEIK

SEQ ID NO: 1021 - protein, 5 aa, synthetic construct
SYAIS

SEQ ID NO: 1022 - protein, 17 aa, synthetic construct
SIIPIFGTANYAQKFQG

SEQ ID NO: 1023 - protein, 16 aa, synthetic construct
GPSEVGAILGYVWFDP

SEQ ID NO: 1024 - protein, 16 aa, synthetic construct
RSSQSLLHSNGYNYLD

SEQ ID NO: 1025 - protein, 7 aa, synthetic construct
LGSNRAS

SEQ ID NO: 1026 - protein, 9 aa, synthetic construct
MQARRIPIT

SEQ ID NO: 1027 - protein, 125 aa, synthetic construct
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCARGPSEVGAILGYVWFDPWGQGTLVTVSS

SEQ ID NO: 1028 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQARRIPITFGGGTKVEIK

SEQ ID NO: 1029 - protein, 5 aa, synthetic construct
NYDIN

SEQ ID NO: 1030 - protein, 17 aa, synthetic construct
WIYPGDGSTKYNEKFKA

SEQ ID NO: 1031 - protein, 8 aa, synthetic construct
GYEDAMDY

SEQ ID NO: 1032 - protein, 11 aa, synthetic construct
KASQDINSYLS

SEQ ID NO: 1033 - protein, 7 aa, synthetic construct
RANRLVD

SEQ ID NO: 1034 - protein, 9 aa, synthetic construct
LQYDEFPLT

SEQ ID NO: 1035 - protein, 117 aa, synthetic construct
QVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSS

SEQ ID NO: 1036 - protein, 106 aa, synthetic construct
DIQMTQSPSSLSASVGDRVTINCKASQDINSYLSWFQQKPGKAPKTLIYRANRVDGVPSRFSGSGSGQDYTLTISSLQPEDFATYYCLQYDEFPLTFGGGTKVEIK

SEQ ID NO: 1037 - protein, 5 aa, synthetic construct
DYYIH

SEQ ID NO: 1038 - protein, 17 aa, synthetic construct
YINPNSGYTNYAQKFQG

SEQ ID NO: 1039 - protein, 12 aa, synthetic construct
YMWERVTGFFDF

SEQ ID NO: 1040 - protein, 11 aa, synthetic construct
LASEDISDDLA

SEQ ID NO: 1041 - protein, 7 aa, synthetic construct
TTSSLQS

SEQ ID NO: 1042 - protein, 9 aa, synthetic construct
QQTYKFPPT

SEQ ID NO: 1043 - protein, 121 aa, synthetic construct
QVQLVQSGAEVKKPGASVKLSCKASGYTFTDYYIHWVRQAPGQGLEWIGYINPNSGYTNYAQKFQGRATMTADKSINTAYVELSRLRSDDTAVYFCTRYMWERVTGFFDFWGQGTMVTVSS

SEQ ID NO: 1044 - protein, 107 aa, synthetic construct
DIQMTQSPSSVSASVGDRVTITCLASEDISDDLAWYQQKPGKAPKVLVYTTSSLQSGVPSRFSGSGSGTDFTLTISSLQPEDFATYFCQQTYKFPPTFGGGTKVEIK